(19) United States
(12) Patent Application Publication
(10) Pub. No.: US 2025/0282754 A1
(43) Pub. Date: Sep. 11, 2025

(54) BICYCLIC COMPOUNDS AND THEIR USE AS PEST CONTROL AGENTS

(71) Applicant: Pl Industries Ltd., Rajasthan Udaipur (IN)

(72) Inventors: Ajay Singh YADAV, Hariyana Gurgaon (IN); Amit Kumar DUTTA, West Bengal Howrah (IN); Siva Nagendra Kumar MOTAPOTHULA, Udaipur (IN); Bhagwan Lal GURJAR, Udaipur (IN); Pritesh Ramesh JAIN, Dhule, Maharashtra (IN); Rohit SAXENA, Uttar Pradesh Lucknow (IN); Sachin Harilal PACHOLE, Aurangabad (IN); Ravikumar Suryanarayana SARAGUR, Karnataka, Bangalore (IN); Alexander G.M. KLAUSENER, Pulheim (DE)

(21) Appl. No.: 18/863,610

(22) PCT Filed: May 10, 2023
(86) PCT No.: PCT/IN2023/050445

§ 371 Date: Nov. 6, 2024

(30) Foreign Application Priority Data
May 11, 2022 (IN) 202211027227

(51) Int. Cl. C07D 401/14; A01N 43/56; A01N 43/60; A01N 43/653; A01N 43/90; A01P 7/04; C07D 401/04; C07D 403/04; C07D 471/04

(52) U.S. Cl. CPC C07D 401/14; A01N 43/56; A01N 43/60; A01N 43/653; A01N 43/90; A01P 7/04; C07D 401/04; C07D 403/04; C07D 471/04

(57) ABSTRACT

Novel bicyclic compounds have formula (I),

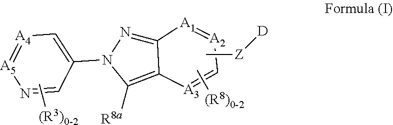

In Formula (I), the definition of A₁, A₂, A₃, A₄, A₅, Z, D, R³, R⁸ and R^{8a} is described in the detailed description. which are demonstrating high pesticidal efficacy. Also described are methods of preparation of compounds of formula (I). The disclosure further relates to compositions, combinations, uses and methods of application of the compounds of formula (I).

### FIELD OF THE INVENTION

The present invention described herein relates to novel bicyclic compounds, in particular the present invention relates to the compounds of formula (I), compositions containing these compounds and their use as pest control agents. Further, the present invention also relates to the preparation of these novel bicyclic compounds and to useful intermediates thereof.

### BACKGROUND

It is known from prior art, for example, from WO2018011111, WO2016071499, WO2015038503, WO2016087363, WO2016087368, WO2016087421 and WO2016087422 that certain substituted bicyclic compounds exhibit pest control properties.

The active compounds reported in the prior art mentioned above have disadvantages in certain aspects. For example, they exhibit narrow spectrum of pesticidal activity only, do not have satisfactory pest control efficacy at lower application rates, or do not exhibit the desired quick action or a long-lasting activity.

Therefore, there is a constant need for new pest control agents with improved pesticidal activity, broader spectrum of efficacy, longer lasting activity, increased plant compatibility, better environmental safety, and improved formulation properties as well as a low risk of resistance development.

### SUMMARY

The present invention relates to novel bicyclic compounds which have been found to have advantageous effects over similar compounds reported in the literature, either in terms of improved pest control activity, more favorable biological or environmental properties, broader spectrum of application or enhanced plant compatibility.

The novel bicyclic compounds of the present invention can also be used in combination with other biologically active compounds, by this improving the efficacy particularly against insects which are difficult to control.

Accordingly, the present invention provides novel bicyclic compounds of formula (I),

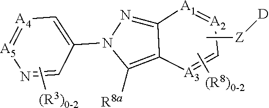

wherein,
Z is selected from a direct bond or —C(═O)—;
D is selected from the group consisting of D₁, D₂ and D₃,

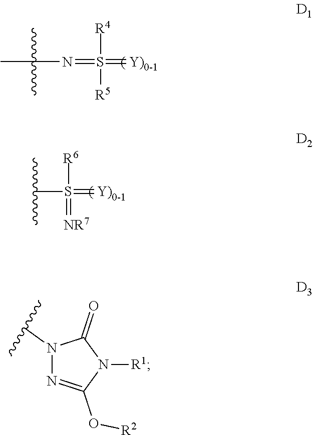

Y represents O or NR²;
A₁, A₂ and A₃ are independently C or N;
A₄ and A₅ are independently C or N, provided that both A₄ and A₅ simultaneously cannot be N;
R¹ is selected from the group consisting of C₁-C₆ alkyl, C₂-C₆ alkenyl, C₂-C₆ alkynyl, C₁-C₆ haloalkyl, C₃-C₈ cycloalkyl, C₃-C₈ cycloalkyl-C₁-C₆ alkyl, phenyl, benzyl, phenyl ethyl and C₂-C₆ heterocyclyl;
R² is selected from the group consisting of C₁-C₆ alkyl, C₂-C₆ alkenyl, C₂-C₆ alkynyl, C₁-C₆ haloalkyl, and C₃-C₈ cycloalkyl-C₁-C₆ alkyl;
R³ is selected from the group consisting of hydrogen, halogen, cyano, C₁-C₆ alkyl, C₂-C₆ alkenyl, C₂-C₆ alkynyl, C₃-C₆-cycloalkyl, C₁-C₆-alkoxy and C₁-C₆haloalkyl;
R⁴ is selected from the group consisting of C₁-C₆ alkyl, C₂-C₆ alkenyl, C₂-C₆ alkynyl, C₁-C₆ haloalkyl, C₂-C₆ haloalkenyl, C₃-C₈ cycloalkyl, C₃-C₈ cycloalkyl-C₁-C₆ alkyl and —NR^{c}R^{d};
   R^{c} is selected from the group consisting of hydrogen, C₁-C₆ alkyl, C₁-C₆ haloalkyl, and C₃-C₈ cycloalkyl;
   R^{d} is selected from the group consisting of hydrogen, C₁-C₆ alkyl, C₂-C₆ alkenyl, C₂-C₆ alkynyl, C₁-C₆haloalkyl, C₂-C₆ haloalkenyl, C₃-C₈ cycloalkyl, C₃-C₆-cycloalkyl-C₁-C₄-alkyl;
   or
   R^{c} and R^{d} substituents together with the atom to which they are attached or together with further atoms selected from the group consisting of C, N, O, C(═O), C(═S), and S(O)₀₋₂ may form a 3- to 6-membered ring, which may optionally be substituted by one or more substituents selected from the group consisting of halogen, CN and C₁-C₆ alkyl;
R⁵ is selected from the group consisting of C₁-C₆ alkyl, C₁-C₆ haloalkyl, C₃-C₈ cycloalkyl, C₃-C₈ cycloalkyl-C₁-C₆ alkyl, C₂-C₆ alkenyl, C₂-C₆ haloalkenyl, phenyl, benzyl, and C₂-C₆ heterocyclyl;
or
R⁴ and R⁵ substituents together with the atom to which they are attached or together with further atoms selected from the group consisting of C, N, O, C(═O), C(═S), and S(O)₀₋₂ may form a 3- to 6-membered ring, which may optionally be substituted by one or more substituents selected from the group consisting of halogen, CN and C₁-C₆ alkyl;
R⁶ is selected from the group consisting of C₁-C₆ alkyl, C₂-C₆ alkenyl, C₂-C₆ alkynyl, C₁-C₆ haloalkyl, C₂-C₆ haloalkenyl, C₃-C₈ cycloalkyl, C₃-C₈ cycloalkyl-C₁-C₆ alkyl, phenyl, benzyl, C₂-C₆ heterocyclyl and —NR^{c}R^{d};
R⁷ is selected from the group consisting of hydrogen, cyano, C₁-C₆ alkyl, C₂-C₆ alkenyl, C₂-C₆ alkynyl, C₁-C₆ haloalkyl, C₂-C₆ haloalkenyl, C₁-C₆-alkoxy, C₃-C₈ cycloalkyl, C₃-C₈ cycloalkyl-C₁-C₆ alkyl, phenyl, benzyl, C₂-C₆ heterocyclyl, C₂-C₆ heterocyclyl-C₁-C₆ alkyl, —COR⁵, —CONR^{c}R^{d}, SCF₃, and —SO₂R⁵;
R⁸ and R^{8a} are independently selected from the group consisting of hydrogen, halogen, cyano, C₁-C₄-alkyl, and C₁-C₄-haloalkyl;
   wherein each aliphatic group of R¹, R², R³, R⁴, R⁵, R⁶ and R⁷ may be optionally substituted with one or more groups of R^{a}; and cyclic groups of R¹, R², R³, R⁴, R⁵, R⁶ and R⁷ may optionally be substituted with one or more groups of R, wherein,
      R^{a} is selected from the group consisting of halogen, cyano, C₁-C₆ alkyl, C₁-C₆ haloalkyl, C₃-C₈ cycloalkyl, C₁-C₆ alkoxy, C₁-C₆ alkylthio, phenyl and C₂-C₆ heterocyclyl;
      R^{b} is selected from the group consisting of halogen, cyano, C₁-C₆ alkyl, C₂-C₆ alkenyl, C₂-C₆ alkynyl, C₁-C₆ haloalkyl, C₂-C₆ haloalkenyl, C₃-C₈ cycloalkyl, C₁-C₆ alkylthio, C₁-C₆ haloalkylthio, C₁-C₆ alkoxy and C₁-C₆ haloalkoxy;
or salts, stereoisomers, tautomers, polymorphs, metal complexes or N-oxides thereof.

In another embodiment, the present invention provides a composition comprising the compound of formula (I), its salts, metal complexes, stereoisomers, polymorphs or N-oxides thereof and at least one additional component selected from the group consisting of surfactants and auxiliaries.

In yet another embodiment, the present invention provides a composition, wherein the said composition additionally comprises at least one biological active compatible compound selected from fungicides, insecticides, nematicides, acaricides, biopesticides, herbicides, plant growth regulators, biostimulants, antibiotics, fertilizers or nutrients.

In yet another embodiment, the present invention provides a method for the preparation of compounds of formula (I).

The present invention will now be described in detail.

### DETAILED DESCRIPTION OF THE INVENTION

### Definitions

The definitions provided herein for the terminologies used in the present disclosure are for illustrative purpose only and in no manner limit the scope of the present invention disclosed in the present disclosure.

As used herein, the terms “comprise”, “comprising”, “include”, “including”, “has”, “having”, “contain”, “containing”, “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components or elements, in addition to those literally disclosed, provided that these additional materials, steps, features, components or elements do not materially affect the basic and novel characteristic(s) of the claimed invention. The term “consisting essentially of” occupies a middle ground between “comprising” and “consisting of”.

Further, unless expressly stated to the contrary, “or” refers to an inclusive “or” and not to an exclusive “or”. For example, a condition A “or” B is satisfied by any one of the following: A is true (or present) and B is false (or not present), A is false (or not present) and B is true (or present), and both A and B are true (or present).

Also, the indefinite articles “a” and “an” preceding an element or component of the present invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in this disclosure, the term “invertebrate pest” or “invertebrates” includes arthropods, gastropods and nematodes of economic importance as pests. The term “arthropod” includes insects, mites, spiders, scorpions, centipedes, millipedes, pill bugs and symphylans. The term “gastropod” includes snails, slugs and other Stylommatophora. The term “nematode” refers to a living organism of the Phylum Nematoda. The term “helminths” includes roundworms, heartworms, phytophagous nematodes (Nematoda), flukes (Tematoda), acanthocephala and tapeworms (Cestoda).

In the context of this disclosure “invertebrate pest control” means inhibition of invertebrate pest development (including mortality, feeding reduction, and/or mating disruption), and related expressions are defined analogously.

The term “agronomic” refers to the production of field crops such as for food, feed and fiber and includes the growth of corn, soybeans and other legumes, rice, cereal (e.g., wheat, oats, barley, rye, rice, maize), leafy vegetables (e.g., lettuce, cabbage, and other vegetable crops), fruiting vegetables (e.g., tomatoes, pepper, eggplant, crucifers and cucurbits), potatoes, sweet potatoes, grapes, cotton, tree fruits (e.g., pome, stone and citrus), small fruit (berries, cherries) and other specialty crops (e.g., canola, sunflower, olives).

The term “non-agronomic” refers to other than field crops, such as horticultural crops (e.g., greenhouse, nursery or ornamental plants not grown in a field), residential, agricultural, commercial and industrial structures, turf (e.g., sod farm, pasture, golf course, lawn, sports field, etc.), wood products, stored product, agro-forestry and vegetation management, public health (i.e. human) and animal health (e.g., domesticated animals such as pets, livestock and poultry, undomesticated animals such as wildlife) applications.

Non-agronomic applications include protecting an animal from an invertebrate parasitic pest by administering a parasiticidally effective (i.e. biologically effective) amount of a compound of the present invention, typically in the form of a composition formulated for veterinary use, to the animal to be protected. As referred to in the present disclosure and claims, the terms “parasiticidal” and “parasiticidally” refers to observable effects on an invertebrate parasite pest to provide protection of an animal from the pest. Parasiticidal effects typically relate to diminishing the occurrence or activity of the target invertebrate parasitic pest. Such effects on the pest include death, retarded growth, diminished mobility or lessened ability to remain on or in the host animal, reduced feeding and inhibition of reproduction. These effects on invertebrate parasite pests provide control (including prevention, reduction or elimination) of a parasitic infestation or infection of the animal.

Compounds of the present disclosure may be present either in a pure form or as mixtures of different possible isomeric forms such as stereoisomers or constitutional isomers. The various stereoisomers include enantiomers, diastereomers, chiral isomers, atropisomers, conformers, rotamers, tautomers, optical isomers, polymorphs, and geometric isomers. Any desired mixtures of these isomers fall within the scope of the claims of the present disclosure. One skilled in the art will appreciate that one stereoisomer may be more active and/or may exhibit beneficial effects when enriched relative to the other isomer(s) or when separated from the other isomer(s). Additionally, the person skilled in the art knows processes or methods or technology to separate, enrich, and/or to selectively prepare said isomers.

The meaning of various terms used in the description shall now be illustrated.

The term -“alkyl”, used either alone or in compound words such as “alkylthio” or “haloalkyl” or —N(alkyl) or alkylcarbonylalkyl or alkylsufonylamino includes straight-chain or branched C₁ to C₁₂ alkyl, preferably C₁ to C₈ alkyl, more preferably C₁ to C₆ alkyl. Representative examples of alkyl include methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1,2,2-trimethylpropyl, 1-ethyl-1-methylpropyl and 1-ethyl-2-methylpropyl or the different isomers. If the alkyl is at the end of a composite substituent, as, for example, in alkylcycloalkyl, the part of the composite substituent at the start, for example the cycloalkyl, may be mono- or polysubstituted identically or differently and independently by alkyl. The same also applies to composite substituents in which other radicals, for example alkenyl, alkynyl, hydroxyl, halogen, carbonyl, carbonyloxy and the like, are at the end.

The term “alkenyl”, used either alone or in compound words includes straight-chain or branched C₂ to C₁₂ alkenes, preferably C₂ to C₈ alkenes, more preferably C₂ to C₆ alkenes. Representative examples of alkenes include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 1,2-trimethyl-2-propenyl, 1-ethyl-1-methyl-2-propenyl, 1-ethyl-2-methyl-1-propenyl and 1-ethyl-2-methyl-2-propenyl and the different isomers. “Alkenyl” also includes polyenes such as 1,2-propadienyl and 2,4-hexadienyl. This definition also applies to alkenyl as a part of a composite substituent, for example haloalkenyl and the like, unless defined specifically elsewhere.

Non-limiting examples of alkynes include ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl and the different isomers. This definition also applies to alkynyl as a part of a composite substituent, for example haloalkynyl etc., unless specifically defined elsewhere. The term “alkynyl” can also include moieties comprised of multiple triple bonds such as 2,5-hexadiynyl.

The term “cycloalkyl” means alkyl closed to form a ring. Non-limiting examples include cyclopropyl, cyclobutyl cyclopentyl cyclohexyl, and cycloheptyl. This definition also applies to cycloalkyl as a part of a composite substituent, for example cycloalkylalkyl etc., unless specifically defined elsewhere.

The term “cycloalkoxy”, “cycloalkenyloxy” and the like are defined analogously. Non-limiting examples of cycloalkoxy include cyclopropyloxy, cyclopentyloxy and cyclohexyloxy. This definition also applies to cycloalkoxy as a part of a composite substituent, for example cycloalkoxy alkyl etc., unless specifically defined elsewhere.

The term “halogen”, either alone or in compound words such as “haloalkyl”, includes fluorine, chlorine, bromine or iodine. Further, when used in compound words such as “haloalkyl”, said alkyl may be partially or fully substituted with halogen atoms which may be the same or different. Non-limiting examples of “haloalkyl” include chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 1,1-dichloro-2,2,2-trifluoroethyl, and 1,1,1-trifluoroprop-2-yl. This definition also applies to haloalkyl as a part of a composite substituent, for example haloalkylaminoalkyl etc., unless specifically defined elsewhere.

The terms “haloalkenyl”, “haloalkynyl” are defined analogously except that, instead of alkyl groups, alkenyl and alkynyl groups are present as a part of the substituent.

The term “haloalkoxy” means straight-chain or branched alkoxy groups where some or all of the hydrogen atoms in these groups may be replaced by halogen atoms as specified above. Non-limiting examples of haloalkoxy include chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy and 1,1,1-trifluoroprop-2-oxy. This definition also applies to haloalkoxy as a part of a composite substituent, for example haloalkoxyalkyl etc., unless specifically defined elsewhere.

The term “haloalkylthio” means straight-chain or branched alkylthio groups where some or all of the hydrogen atoms in these groups may be replaced by halogen atoms as specified above. Non-limiting examples of haloalkylthio include chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio, pentafluoroethylthio and 1,1,1-trifluoroprop-2-ylthio. This definition also applies to haloalkylthio as a part of a composite substituent, for example haloalkylthioalkyl etc., unless specifically defined elsewhere.

The term “hydroxy” means —OH, amino means —NRR, wherein R can be H or any possible substituent such as alkyl. Carbonyl means —C(O)—, carbonyloxy means —OC(O)—, sulfinyl means SO, sulfonyl means S(O)₂.

The term “alkoxy” used either alone or in compound words includes C₁ to C₁₂ alkoxy, preferably C₁ to C₈ alkoxy, more preferably C₁ to C₆ alkoxy. Examples of alkoxy include methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 2,2-dimethylpropoxy, 1-ethylpropoxy, hexoxy, 1,1-dimethylpropoxy, 1,2-dimethylpropoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy and the different isomers. This definition also applies to alkoxy as a part of a composite substituent, for example haloalkoxy, alkynylalkoxy, etc., unless specifically defined elsewhere.

The term “Alkoxyalkyl” denotes alkoxy substitution on alkyl. Non-limiting examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂.

The term “alkylthio” includes branched or straight-chain alkylthio moieties such as methylthio, ethylthio, propylthio, 1-methylethylthio, butylthio, 1-methylpropylthio, 2-methylpropylthio, 1,1-dimethylethylthio, pentylthio, 1-methylbutylthio, 2-methylbutylthio, 3-methylbutylthio, 2,2-dimethylpropylthio, 1-ethylpropylthio, hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio and the different isomers.

Halocycloalkyl, halocycloalkenyl, alkylcycloalkyl, cycloalkylalkyl, cycloalkoxyalkyl, alkylsulfinylalkyl, alkylsulfonylalkyl, haloalkylcarbonyl, cycloalkylcarbonyl, haloalkoxylalkyl, and the like, are defined analogously to the above examples.

“Alkylamino”, “dialkylamino”, and the like, are defined analogously to the above examples.

The term “carbocycle” includes an “aromatic carbocyclic ring system” and a “nonaromatic carbocyclic ring system” or polycyclic or bicyclic (spiro, fused, bridged, nonfused) ring compounds in which the ring may be aromatic or non-aromatic (where aromatic indicates that the Hueckel rule is satisfied and non-aromatic indicates that the Hueckel rule is not satisfied).

The term “hetero” in connection with rings refers to a ring in which at least one ring atom is not carbon and which can contain 1 to 4 heteroatoms independently selected from the group consisting of nitrogen, oxygen and sulfur, provided that each ring contains no more than 4 nitrogens, no more than 2 oxygens and no more than 2 sulfurs.

The term “heterocyclyl” or “heterocyclic” includes an “aromatic heterocycle” or a “heteroaryl ring system” and a “nonaromatic heterocycle ring system” or polycyclic or bicyclic (spiro, fused, bridged, non-fused) ring compounds in which the ring may be aromatic or non-aromatic, wherein the heterocycle ring contains at least one heteroatom selected from N, O, S(O)₀₋₂, or one of the C ring member of the heterocycle may be replaced by a heteroatom and optionally with C(═O), C(═S), C(=CR*R*) and C=NR*, * indicates integers.

The term “non-aromatic heterocycle” or “non-aromatic heterocyclic” means a three- to fifteen-membered, preferably three- to ten-membered, saturated or partially unsaturated heterocycle containing one to four heteroatoms from the group of oxygen, nitrogen and sulphur: mono, bi- or tricyclic heterocycles which contain, in addition to carbon ring members, one to three nitrogen atoms and/or one oxygen or sulphur atom or one or two oxygen and/or sulphur atoms; if the ring contains more than one oxygen atom, they are not directly adjacent; for example (but not limited to) oxetanyl, oxiranyl, aziridinyl, thietanyl, tetrahydrofuranyl, tetrahydrothienyl, pyrrolidinyl, isoxazolidinyl, isothiazolidinyl, pyrazolidinyl, oxazolidinyl, thiazolidinyl, imidazolidinyl, 1,2,4-oxadiazolidinyl, 1,2,4-thiadiazolidinyl, 1,2,4-triazolidin-1-yl, 1,2,4-triazolidin-3-yl, 1,2,3-triazolidinyl, 1,3,4-oxadiazolidinyl, 1,3,4-thiadiazolidinyl, 1,3,4-triazolidinyl, dihydrofuryl, dihydrothienyl, pyrrolinyl, isoxazolinyl, isothiazolinyl, dihydropyrazolyl, dihydrooxazolyl, dihydrothiazolyl, piperidinyl, pyrazynyl, morpholinyl, thiomorphlinyl, 1,3-dioxan-5-yl, tetrahydropyranyl, tetrahydrothienyl, hexahydropyridazinyl, hexahydropyrimidinyl, piperazinyl and cycloserines. This definition also applies to heterocyclyl as a part of a composite substituent, for example heterocyclylalkyl etc., unless specifically defined elsewhere.

The term “heteroaryl” or “aromatic heterocyclic” means 5 or 6-membered, fully unsaturated monocyclic ring systems containing one to four heteroatoms from the group of oxygen, nitrogen and sulphur; if the ring contains more than one oxygen atom, they are not directly adjacent; 5-membered heteroaryl containing one to four nitrogen atoms or one to three nitrogen atoms and one sulphur or oxygen atom: 5-membered heteroaryl groups which, in addition to carbon atoms, may contain one to four nitrogen atoms or one to three nitrogen atoms and one sulphur or oxygen atom as ring members, for example (but not limited thereto) furyl, thienyl, pyrrolyl, isoxazolyl, isothiazolyl, pyrazolyl, oxazolyl, thiazolyl, imidazolyl, 1,2,4-oxadiazolyl, 1,2,4-thiadiazolyl, 1,2,4-triazolyl, 1,3,4-oxadiazolyl, 1,3,4-thiadiazolyl, 1,3,4-triazolyl, tetrazolyl; nitrogen-bonded 5-membered heteroaryl containing one to four nitrogen atoms, or benzo fused nitrogen-bonded 5-membered heteroaryl containing one to three nitrogen atoms: 5-membered heteroaryl groups which, in addition to carbon atoms, may contain one to four nitrogen atoms or one to three nitrogen atoms as ring members and in which two adjacent carbon ring members or one nitrogen and one adjacent carbon ring member may be bridged by a buta-1,3-diene-1,4-diyl group in which one or two carbon atoms may be replaced by nitrogen atoms, where these rings are attached to the skeleton via one of the nitrogen ring members, for example (but not limited to) 1-pyrrolyl, 1-pyrazolyl, 1,2,4-triazol-1-yl, 1-imidazolyl, 1,2,3-triazol-1-yl and 1,3,4-triazol-1-yl. 6-membered heteroaryl which contains one to four nitrogen atoms: 6-membered heteroaryl groups which, in addition to carbon atoms, may contain, respectively, one to three and one to four nitrogen atoms as ring members, for example (but not limited thereto) pyridinyl, pyridazinyl, pyrimidinyl, pyrazinyl, 1,3,5-triazin-2-yl, 1,2,4-triazin-3-yl and 1,2,4,5-tetrazin-3-yl; benzofused 5-membered heteroaryl containing one to three nitrogen atoms or one nitrogen atom and one oxygen or sulphur atom: for example (but not limited to) indolyl, benzimidazolyl, indazolyl, benzofuranyl, benzothiophenyl, benzothiazolyl, and benzoxazolyl; benzofused 6-membered heteroaryl which contains one to three nitrogen atoms: for example (but not limited to) quinolinyl, isoquinolinyl, quinoxalinyl, phthalazinyl, quinazolinyl and cinnolinyl.

The term “partially/fully saturated or unsaturated heterocycles” include partially/fully saturated heterocycles or partially/fully unsaturated heterocycles.

This definition also applies to heteroaryl as a part of a composite substituent, for example heteroarylalkyl etc., unless specifically defined elsewhere.

Non-limiting examples of “alkylcarbonyl” include C(O)CH₃, C(O)CH₂CH₂CH₃ and C(O)CH(CH₃)₂.

Non-limiting examples of “alkoxycarbonyl” include CH₃OC(═O), CH₃CH₂OC(═O), CH₃CH₂CH₂OC(═O), (CH₃)₂CHOC(═O) and the different butoxy- or pentoxycarbonyl isomers.

The term “amide” means A-R′C═ONR″—B, wherein R′ and R″ indicate substituents and A and B indicate any group.

The term “thioamide” means A-R′C═SNR″—B, wherein R′ and R″ indicate substituents and A and B indicate any group.

The total number of carbon atoms in a substituent group is indicated by the “Cᵢ-Cⱼ” prefix where i and j are numbers from 1 to 18. For example, C₁-C₃ alkylsulfonyl designates methylsulfonyl through propylsulfonyl; C₂ alkoxyalkyl designates CH₃OCH₂; C₃ alkoxyalkyl designates, for example, CH₃CH(OCH₃), CH₃OCH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂OCH₂ and CH₃CH₂OCH₂CH₂. In the above recitations, when a compound of formula (I) is comprised of one or more heterocyclic rings, all substituents are attached to these rings through any available carbon or nitrogen by replacement of a hydrogen on said carbon or nitrogen.

When a compound is substituted with a substituent bearing a subscript that indicates the number of said substituents can exceed 1, said substituents (when they exceed 1) are independently selected from the group of defined substituents. Further, when the subscript m in (R)ₘ indicates an integer ranging from for example 0 to 4 then the number of substituents may be selected from the integers between 0 and 4 inclusive.

When a group contains a substituent which can be hydrogen, then, when this substituent is taken as hydrogen, it is recognized that said group is being un-substituted.

The term “optionally substituted” is used herein interchangeably with the phrase “substituted or unsubstituted” or with the term “(un)substituted”. Unless otherwise indicated, an optionally substituted group may have a substituent at each substitutable position of the group, and each substitution is independent of the other. An optionally substituted group also may have no substituents. Therefore, the phrase “optionally substituted with one or more substituents” means that the number of substituents may vary from zero up to the number of available positions for substitution.

The term (un)substituted is referred as said groups are unsubstituted or said groups are independently substituted with the substituents selected from the group consisting of halogen, cyano, C₁-C₆ alkyl, C₂-C₆ alkenyl, C₂-C₆ alkynyl, C₁-C₆ haloalkyl, C₃-C₈ cycloalkyl, C₁-C₆-alkoxy, NH(alkyl), N(alkyl)₂ and —S(O)₀₋₂C₁-C₆ alkyl.

The term “aliphatic group” used either alone or in compound words includes straight-chain or branched substituents e.g C₁-C₆ alkyl, C₂-C₆ alkenyl, C₂-C₆ alkynyl, C₁-C₆ haloalkyl, C₂-C₆ haloalkenyl, C₁-C₆ alkylthio, C₁-C₆haloalkylthio, C₁-C₆ alkoxy or C₁-C₆ haloalkoxy;

The term “cyclic groups” used either alone or in compound words includes fully/partially saturated or unsaturated cyclic substitutions or groups e.g cycloalkyl, phenyl, C₂-C₆ heterocyclyl etc.

The embodiments herein and the various features and advantageous details thereof are explained with reference to the non-limiting embodiments in the description. Descriptions of well-known components and processing techniques are omitted so as to not unnecessarily obscure the embodiments herein. The examples used herein are intended merely to facilitate an understanding of ways in which the embodiments herein may be practiced and to further enable those of skilled in the art to practice the embodiments herein. Accordingly, the examples should not be construed as limiting the scope of the embodiments herein.

The description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

Any discussion of documents, acts, materials, devices, articles and the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values mentioned in the description and the description/claims though might form a critical part of the present invention, any deviation from such numerical values shall still fall within the scope of the present invention if that deviation follows the same scientific principle as that of the present invention disclosed in the present invention. The inventive compound of the present invention may, if appropriate, be present as mixtures of different possible isomeric forms, especially of stereoisomers, for example E and Z, threo and erythro, and also optical isomers, but if appropriate also of tautomers. Both the E and the Z isomers, and also the threo and erythro isomers, and the optical isomers, any desired mixtures of these isomers and the possible tautomeric forms are disclosed and claimed.

The term “pest” for the purpose of the present disclosure includes but is not limited to fungi, stramenopiles (oomycetes), bacteria, nematodes, mites, ticks, insects and rodents.

The term “plant” is understood here to mean all plants and plant populations, such as desired and undesired wild plants or crop plants (including naturally occurring crop plants). Crop plants may be plants which can be obtained by conventional breeding and optimization methods or by biotechnological and genetic engineering methods or combinations of these methods, including the transgenic plants and including the plant cultivars which are protectable and non-protectable by plant breeders' rights.

In the present disclosure, the term “plant” includes a living organism of the kind exemplified by trees, shrubs, herbs, grasses, ferns, and mosses, typically growing in a site, absorbing water and required substances through its roots, and synthesizing nutrients in its leaves by photosynthesis.

Examples of “plant” for the purpose of the present invention include agricultural crops such as wheat, rye, barley, triticale, oats or rice; beet, e.g. sugar beet or fodder beet; fruits and fruit trees, such as pomes, stone fruits or soft fruits, e.g. apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit and citrus trees, such as oranges, lemons, grapefruits or mandarins; any horticultural plants, vegetables, such as spinach, lettuce, asparagus, cabbages, carrots, onions, tomatoes, potatoes, cucurbits or paprika; lauraceous plants, such as avocados, cinnamon or camphor; cucurbitaceae; oleaginous plants; energy and raw material plants, such as cereals, corn, soybean, other leguminous plants, rape, sugar cane or oil palm; tobacco; nuts; coffee; tea; cacao; bananas; peppers; vines (table grapes and grape juice grape vines); hop; turf; sweet leaf (also called Stevia); natural rubber plants or ornamental and forestry plants, such as flowers, shrubs, broad-leaved trees or evergreens, e.g. conifers; and on the plant propagation material, such as seeds, and the crop material of these plants.

Preferably, the plants for the purpose of the present invention include but are not limited to cereals, corn, rice, soybean and other leguminous plants, fruits and fruit trees, grapes, nuts and nut trees, citrus and citrus trees, any horticultural plants, cucurbitaceae, oleaginous plants, tobacco, coffee, tea, cacao, sugar beet, sugar cane, cotton, potato, tomato, onions, peppers and vegetables, ornamentals, any floricultural plants and other plants for the use of humans and animals.

The term “plant parts” is understood to mean all parts and organs of plants above and below the ground. For the purpose of the present disclosure the term plant part includes but is not limited to cuttings, leaves, twigs, tubers, flowers, seeds, branches, roots including taproots, lateral roots, root hairs, root apex, root cap, rhizomes, slips, shoots, fruits, fruit bodies, bark, stem, buds, auxillary buds, meristems, nodes and internodes.

The term “locus thereof” includes soil, surroundings of plant or plant parts and equipment or tools used before, during or after sowing/planting a plant or a plant part.

Application of the compounds of the present disclosure or the compound of the present disclosure in a composition optionally comprising other compatible compounds to a plant or a plant material or locus thereof include the application by a technique known to a person skilled in the art which includes but is not limited to spraying, coating, dipping, fumigating, impregnating, injecting and dusting.

The term “applied” means adhered to a plant or plant part either physically or chemically including impregnation.

The present invention relates to a compound of formula (I),

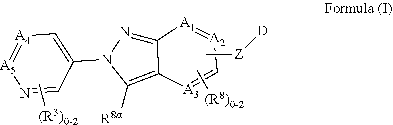

wherein,
Z is selected from a direct bond or —C(═O)—;
D is selected from the group consisting of D₁, D₂ and D₃,

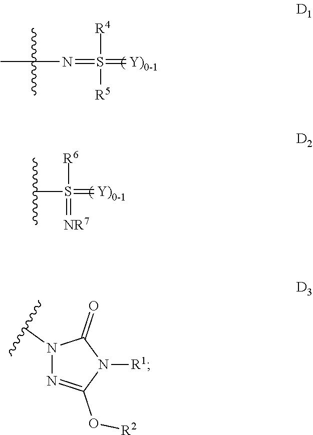

Y represents O or NR⁷;
A₁, A₂ and A₃ are independently C or N;
A₄ and A₅ are independently C or N, provided that both A₄ and A₅ simultaneously cannot be N;
R¹ is selected from the group consisting of C₁-C₆ alkyl, C₂-C₆ alkenyl, C₂-C₆ alkynyl, C₁-C₆ haloalkyl, C₃-C₈ cycloalkyl, C₃-C₈ cycloalkyl-C₁-C₆ alkyl, phenyl, benzyl, phenyl ethyl and C₂-C₆ heterocyclyl;
R² is selected from the group consisting of C₁-C₆ alkyl, C₂-C₆ alkenyl, C₂-C₆ alkynyl, C₁-C₆ haloalkyl, and C₃-C₈ cycloalkyl-C₁-C₆ alkyl;
R³ is selected from the group consisting of hydrogen, halogen, cyano, C₁-C₆ alkyl, C₂-C₆ alkenyl, C₂-C₆ alkynyl, C₃-C₆-cycloalkyl, C₁-C₆-alkoxy and C₁-C₆haloalkyl;
R⁴ is selected from the group consisting of C₁-C₆ alkyl, C₂-C₆ alkenyl, C₂-C₆ alkynyl, C₁-C₆ haloalkyl, C₂-C₆ haloalkenyl, C₃-C₈ cycloalkyl, C₃-C₈ cycloalkyl-C₁-C₆ alkyl and —NR^{c}R^{d};
   R^{c} is selected from the group consisting of hydrogen, C₁-C₆ alkyl, C₁-C₆ haloalkyl, and C₃-C₈ cycloalkyl;
   R^{d} is selected from the group consisting of hydrogen, C₁-C₆ alkyl, C₂-C₆ alkenyl, C₂-C₆ alkynyl, C₁-C₆haloalkyl, C₂-C₆ haloalkenyl, C₃-C₈ cycloalkyl, C₃-C₆-cycloalkyl-C₁-C₄-alkyl;
   or
   R^{c} and R^{d} substituents together with the atom to which they are attached or together with further atoms selected from the group consisting of C, N, O, C(═O), C(═S), and S(O)₀₋₂ may form a 3- to 6-membered ring, which may optionally be substituted by one or more substituents selected from the group consisting of halogen, CN and C₁-C₆ alkyl;
R⁵ is selected from the group consisting of C₁-C₆ alkyl, C₁-C₆ haloalkyl, C₃-C₈ cycloalkyl, C₃-C₈ cycloalkyl-C₁-C₆ alkyl, C₂-C₆ alkenyl, C₂-C₆ haloalkenyl, phenyl, benzyl, and C₂-C₆ heterocyclyl; or
R⁴ and R⁵ substituents together with the atom to which they are attached or together with further atoms selected from the group consisting of C, N, O, C(═O), C(═S), and S(O)₀₋₂ may form a 3- to 6-membered ring, which may optionally be substituted by one or more substituents selected from the group consisting of halogen, CN and C₁-C₆ alkyl;
R⁶ is selected from the group consisting of C₁-C₆ alkyl, C₂-C₆ alkenyl, C₂-C₆ alkynyl, C₁-C₆ haloalkyl, C₂-C₆ haloalkenyl, C₃-C₈ cycloalkyl, C₃-C₈ cycloalkyl-C₁-C₆ alkyl, phenyl, benzyl, C₂-C₆ heterocyclyl and —NR^{c}R^{d};
R⁷ is selected from the group consisting of hydrogen, cyano, C₁-C₆ alkyl, C₂-C₆ alkenyl, C₂-C₆ alkynyl, C₁-C₆ haloalkyl, C₂-C₆ haloalkenyl, C₁-C₆-alkoxy, C₃-C₈ cycloalkyl, C₃-C₈ cycloalkyl-C₁-C₆ alkyl, phenyl, benzyl, C₂-C₆ heterocyclyl, C₂-C₆ heterocyclyl-C₁-C₆ alkyl, —COR⁵, —CONR^{c}R^{d}, SCF₃, and —SO₂R⁵;
R⁸ and R^{8a} are independently selected from the group consisting of hydrogen, halogen, cyano, C₁-C₄-alkyl, and C₁-C₄-haloalkyl;
   wherein each aliphatic group of R¹, R², R³, R⁴, R⁵, R⁶ and R⁷ may be optionally substituted with one or more groups of R^{a}; and cyclic groups of R¹, R², R³, R⁴, R⁵, R⁶ and R⁷ may optionally be substituted with one or more groups of R, wherein,
      R^{a} is selected from the group consisting of halogen, cyano, C₁-C₆ alkyl, C₁-C₆ haloalkyl, C₃-C₈ cycloalkyl, C₁-C₆ alkoxy, C₁-C₆ alkylthio, phenyl and C₂-C₆ heterocyclyl;
      R^{b} is selected from the group consisting of halogen, cyano, C₁-C₆ alkyl, C₂-C₆ alkenyl, C₂-C₆ alkynyl, C₁-C₆ haloalkyl, C₂-C₆ haloalkenyl, C₃-C₈ cycloalkyl, C₁-C₆ alkylthio, C₁-C₆ haloalkylthio, C₁-C₆ alkoxy and C₁-C₆ haloalkoxy;
or salts, stereoisomers, tautomers, polymorphs, metal complexes or N-oxides thereof.

The following embodiments provide definitions, including preferred definitions, for substituents with reference to the compounds of formula (I) according to the invention. For any one of these substituents, any of the definitions given below may be combined with any definition of any other substituent given below or elsewhere in this document.

In one embodiment, the present invention provides compounds of formula (I), wherein D is D₁,

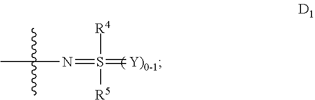

Z, Y, R³, R⁴ and R⁵ are as defined above.

In another embodiment, the present invention provides compounds of formula (I), wherein D is D₂,

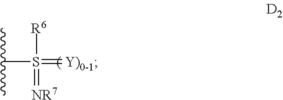

Z is a direct bond, Y, R³, R⁶ and R are as defined above.

In yet another embodiment, the present invention provides compounds of formula (I), wherein D is D₃,

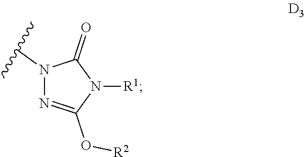

Z, R¹, R² and R³ are as defined above.

In one embodiment, the present invention provides compounds of formula (I), representing compounds of formula (Ia)

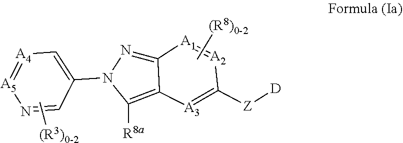

wherein, A₁, A₂, A₃, A₄, A₅, Z, D, R³, R⁸ and R^{8a} are defined above.

In another embodiment, the present invention provides compounds of formula (I), representing compounds of formula (Ib)

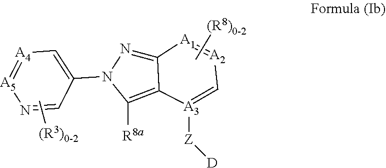

wherein, A₃ is C; A₁, A₂, A₄, A₅, Z, D, R³, R⁸ and R^{8a} are defined above.

In one embodiment the groups R¹, R², R³, R⁴, R⁵, R⁶ and R of the compound of formula (I) are described as:
R¹ is selected from C₁-C₆ alkyl, C₂-C₆ alkenyl, C₂-C₆ alkynyl, C₁-C₆haloalkyl, C₃-C₈ cycloalkyl, C₃-C₈ cycloalkyl-C₁-C₆ alkyl, benzyl, phenyl and C₃-C₆ heterocyclyl; preferably C₁-C₆ alkyl, C₃-C₆ cycloalkyl, phenyl, benzyl, and 4-6 membered aromatic or non-aromatic heterocycles.
R² is selected from C₁-C₆ alkyl, C₂-C₆ alkenyl, C₂-C₆ alkynyl, C₁-C₆haloalkyl, C₃-C₈ cycloalkyl-C₁-C₆ alkyl; preferably C₁-C₆ alkyl, C₃-C₆ cycloalkyl-C₁-C₆ alkyl, more preferably C₁-C₆ alkyl; most preferably methyl or ethyl.
R³ is selected from the group consisting of hydrogen, halogen, cyano, C₁-C₆ alkyl, C₂-C₆ alkenyl, C₂-C₆ alkynyl, C₃-C₆-cycloalkyl, C₁-C₆-alkoxy and C₁-C₆ haloalkyl; preferably hydrogen, halogen, and cyano; C₁-C₃-alkyl, C₁-C₃-haloalkyl, and C₁-C₃-alkoxy; more preferably hydrogen, fluorine, chlorine, methyl, ethyl, methoxy and CF₃
R⁴ is selected from the group consisting of C₁-C₆ alkyl, C₂-C₆ alkenyl, C₂-C₆ alkynyl, C₁-C₆ haloalkyl, C₂-C₆ haloalkenyl, C₃-C₈ cycloalkyl, C₃-C₈ cycloalkyl-C₁-C₆ alkyl and —NR^{c}R^{d}; preferably C₁-C₆ alkyl, C₃-C₆ cycloalkyl-C₁-C₆ alkyl, C₃-C₆ cycloalkyl, —NR^{c}R^{d} and C₁-C₆ haloalkyl; more preferably C₁-C₆ alkyl, C₃-C₆ cycloalkyl, and C₁-C₆haloalkyl;
   R^{c} is selected from the group consisting of hydrogen, C₁-C₆ alkyl, C₃-C₈ cycloalkyl; more preferably hydrogen or C₁-C₆ alkyl;
   R^{d} is selected from the group consisting of C₁-C₆ alkyl, C₁-C₆—C₃—C₈ cycloalkyl-C₁-C₆ alkyl, phenyl and C₂-C₆ heterocyclyl; preferably C₁-C₆alkyl, C₁-C₆haloalkyl, and C₃-C₈ cycloalkyl; or
   R^{c} and R^{d} substituents together with the atom to which they are attached or together with further atoms selected from the group consisting of C, N, O, C(═O), C(═S), and S(O)₀₋₂ may form a 3- to 6-membered ring, which may optionally be substituted by one or more substituents selected from the group consisting of halogen, CN or C₁-C₆ alkyl;
R⁵ is selected from the group consisting of C₁-C₆ alkyl, C₁-C₆ haloalkyl, C₃-C₈ cycloalkyl, C₃-C₈ cycloalkyl-C₁-C₆ alkyl, C₂-C₆ alkenyl, C₂-C₆ haloalkenyl, phenyl, benzyl, and C₂-C₆ heterocyclyl; Optionally, R⁴ and R⁵ substituents together with the atom to which they are attached or together with further atoms selected from the group consisting of C, N, O, C(═O), C(═S), and S(O)₀₋₂ may form a 4- to 6-membered ring, which may optionally be substituted by one or more substituents selected from the group consisting of halogen, CN and C₁-C₆ alkyl;
R⁶ is selected from the group consisting of C₁-C₆ alkyl, C₂-C₆ alkenyl, C₁-C₆ haloalkyl, C₂-C₆ haloalkenyl, C₃-C₈ cycloalkyl, C₃-C₈ cycloalkyl-C₁-C₆ alkyl, and C₂-C₆ heterocyclyl; preferably C₁-C₆ alkyl, C₁-C₆haloalkyl, C₃-C₈ cycloalkyl and C₂-C₆ heterocyclyl.
R⁷ is selected from the group consisting of hydrogen, cyano, C₁-C₆ alkyl, C₂-C₆ alkenyl, C₂-C₆ alkynyl, C₁-C₆haloalkyl, C₂-C₆ haloalkenyl, C₁-C₆-alkoxy, C₃-C₈ cycloalkyl, C₃-C₈ cycloalkyl-C₁-C₆ alkyl, C₂-C₆ heterocyclyl and —COR; preferably hydrogen, cyano, C₁-C₆ alkyl, C₁-C₆ haloalkyl, C₁-C₆-alkoxy, C₃-C₈ cycloalkyl C₂-C₆ heterocyclyl and —COR⁵.
R⁸ and R^{8a} are independently selected from the group consisting of hydrogen, halogen, cyano, C₁-C₆ alkyl, C₁-C₆haloalkyl, C₃-C₈ cycloalkyl, phenyl, and C₂-C₆ heterocyclyl; preferably hydrogen, halogen, cyano, C₁-C₆ alkyl, and C₁-C₆haloalkyl.
wherein each aliphatic group of R¹, R², R⁴, R, R⁶ and R⁷ may be optionally substituted with one or more groups of R^{a} and cyclic groups of R¹, R², R³, R⁴, R, R⁶ and R⁷ may optionally be substituted with one or more groups of R,
   R^{a} is selected from the group consisting of halogen, cyano, C₁-C₆ alkyl, C₁-C₆haloalkyl, C₃-C₈ cycloalkyl, C₁-C₆ alkoxy, C₁-C₆ alkylthio, phenyl and C₂-C₆ heterocyclyl;
   R^{b} is selected from the group consisting of halogen, cyano, C₁-C₆ alkyl, C₂-C₆ alkenyl, C₂-C₆ alkynyl, C₁-C₆ haloalkyl, C₂-C₆ haloalkenyl, C₃-C₈ cycloalkyl, C₁-C₆ alkylthio, C₁-C₆ haloalkylthio, C₁-C₆ alkoxy and C₁-C₆ haloalkoxy.

Further, embodiments of this disclosure described herein, can be combined in any manner, and the descriptions of variables in the embodiments pertain not only to the compounds of formula (I) but also to the starting compounds and to intermediate compounds useful for preparing the compounds of formula (I).

In this sense, embodiments that can be combined according to the present invention, are illustrated in the following:

In one embodiment, the present invention provides compounds of formula (I), wherein,
D is D₁,
Z is a direct bond or —C(═O)—; preferably Z is a direct bond;
R³ is selected from the group consisting of hydrogen, halogen, cyano, C₁-C₆ alkyl, C₁-C₆haloalkyl, and C₁-C₆ alkoxy;
R⁴ is selected from the group consisting of C₁-C₆ alkyl, C₃-C₈ cycloalkyl, C₃-C₈ cycloalkyl-C₁-C₆ alkyl and —NR^{c}R^{d};
   wherein, R^{c} is selected from the group consisting of hydrogen or C₁-C₆ alkyl;
   R^{d} is selected from the group consisting of hydrogen or C₁-C₆ alkyl;
R⁵ is selected from the group consisting of C₁-C₆ alkyl, C₁-C₆ haloalkyl, C₃-C₈ cycloalkyl, C₃-C₈ cycloalkyl-C₁-C₆ alkyl, phenyl, benzyl, and C₂-C₆ heterocyclyl;
or
R⁴ and R⁵ substituents together with the atom to which they are attached or together with further atoms selected from the group consisting of C, N, O, C(═O), C(═S), and S(O)₀₋₂ may form a 4- to 6-membered ring, which may optionally be substituted by one or more substituents selected from the group consisting of halogen, CN and C₁-C₆ alkyl.
   wherein each aliphatic group of R⁴ and R⁵ may be optionally substituted with one or more groups of R^{a} and cyclic groups of R⁴ and R⁵ may optionally be substituted with one or more groups of R^{b},
      R^{a} is selected from the group consisting of halogen, cyano, C₁-C₆ alkyl, C₁-C₆haloalkyl, C₃-C₈ cycloalkyl, C₁-C₆ alkoxy, C₁-C₆ alkylthio, phenyl and C₂-C₆ heterocyclyl;
      R^{b} is selected from the group consisting of halogen, cyano, C₁-C₆ alkyl, C₂-C₆ alkenyl, C₂-C₆ alkynyl, C₁-C₆ haloalkyl, C₂-C₆ haloalkenyl, C₃-C₈ cycloalkyl, C₁-C₆ alkylthio, C₁-C₆ haloalkylthio, C₁-C₆ alkoxy and C₁-C₆ haloalkoxy;
   R⁸ and R^{8a} are independently selected from the group consisting of hydrogen, halogen, cyano, C₁-C₄-alkyl, and C₁-C₄-haloalkyl;
      A₁, A₂, A₃, A₄, A₅, are defined as above;
or salts, stereoisomers, tautomers, polymorphs, metal complexes or N-oxides thereof.

In another embodiment, the present invention provides compounds of formula (I), wherein,
D is D₂,
Z is a direct bond;
R³ is selected from the group consisting of hydrogen, halogen, C₁-C₆ alkyl, C₁-C₆ haloalkyl, and C₁-C₆ alkoxy;
R⁶ is selected from the group consisting of C₁-C₆ alkyl, C₂-C₆ alkenyl, C₂-C₆ alkynyl, C₁-C₆ haloalkyl, C₂-C₆ haloalkenyl, C₃-C₈ cycloalkyl, C₃-C₈ cycloalkyl-C₁-C₆ alkyl, —NR^{c}R^{d}, phenyl, benzyl and C₂-C₆ heterocyclyl; preferably R⁶ is selected from the group consisting of C₁-C₆ alkyl, C₃-C₈ cycloalkyl, C₃-C₅ cycloalkyl-C₁-C₆ alkyl and —NR^{c}R^{d};
   wherein, R^{c} is selected from the group consisting of hydrogen or C₁-C₆ alkyl;
   R^{d} is selected from the group consisting of hydrogen or C₁-C₆ alkyl;
R⁷ is independently selected from the group consisting of hydrogen, cyano, C₁-C₆ alkyl, C₂-C₆ alkenyl, C₂-C₆ alkynyl, C₁-C₆ haloalkyl, C₂-C₆ haloalkenyl, C₁-C₆-alkoxy, C₃-C₈ cycloalkyl, C₃-C₈ cycloalkyl-C₁-C₆ alkyl, phenyl, benzyl, C₂-C₆ heterocyclyl, C₂-C₆ heterocyclyl C₁-C₆ alkyl and —COR⁵; preferably
R⁷ is selected from the group consisting of hydrogen, C₁-C₆ alkyl, C₁-C₆ haloalkyl, C₃-C₈ cycloalkyl, C₃-C₈ cycloalkyl-C₁-C₆ alkyl, —COR, phenyl, benzyl, C₂-C₆ heterocyclyl and cyano;
   wherein, R⁵ is selected from the group consisting of C₁-C₆ alkyl and C₃-C₈ cycloalkyl;
   wherein each aliphatic group of R⁵, R⁶ and R⁷ may be optionally substituted with one or more groups of R^{a}; and cyclic groups of R⁵, R⁶ and R⁷ may optionally be substituted with one or more groups of R^{b}, wherein,
      R^{a} is selected from the group consisting of halogen, cyano, C₁-C₆ alkyl, C₁-C₆haloalkyl, C₃-C₈ cycloalkyl, C₁-C₆ alkoxy, C₁-C₆ alkylthio, phenyl and C₂-C₆ heterocyclyl;
      R^{b} is selected from the group consisting of halogen, cyano, C₁-C₆ alkyl, C₂-C₆ alkenyl, C₂-C₆ alkynyl, C₁-C₆ haloalkyl, C₂-C₆ haloalkenyl, C₃-C₈ cycloalkyl, C₁-C₆ alkylthio, C₁-C₆ haloalkylthio, C₁-C₆ alkoxy and C₁-C₆ haloalkoxy; A₁, A₂, A₃, A₄, and A₅ are as defined above;
or salts, stereoisomers, tautomers, polymorphs, metal complexes or N-oxides thereof.

In yet another embodiment, the present invention provides compounds of formula (I), wherein,
D is D₃;
Z is a direct bond;
R₂ is C₁-C₆ alkyl;
R³ is selected from the group consisting of hydrogen, halogen, C₁-C₆ alkyl, C₁-C₆ haloalkyl, and C₁-C₆ alkoxy;
R₁ is selected from C₁-C₆alkyl, C₂-C₆ alkenyl, C₂-C₆ alkynyl, C₁-C₆ haloalkyl, C₃-C₆ cycloalkyl, C₃-C₆ cycloalkyl-C₁-C₆ alkyl, phenyl, benzyl, phenyl ethyl and 4-6 membered heterocyclyl;
each aliphatic group of R¹, may be optionally substituted with one or more groups of R^{a} and cyclic groups of R¹ and may optionally be substituted with one or more groups of R, wherein,
   R^{a} is selected from the group consisting of halogen, cyano, C₁-C₆ alkyl, C₁-C₆ haloalkyl, C₃-C₈ cycloalkyl, C₁-C₆ alkoxy, C₁-C₆ alkylthio, phenyl and C₂-C₆ heterocyclyl;
   R^{b} is selected from the group consisting of halogen, cyano, C₁-C₆ alkyl, C₂-C₆ alkenyl, C₂-C₆ alkynyl, C₁-C₆ haloalkyl, C₂-C₆ haloalkenyl, C₃-C₈ cycloalkyl, C₁-C₆ alkylthio, C₁-C₆ haloalkylthio, C₁-C₆ alkoxy and C₁-C₆ haloalkoxy;
   R⁸ and R^{8a} are independently selected from the group consisting of hydrogen, halogen, cyano, C₁-C₄-alkyl, and C₁-C₄-haloalkyl;
or salts, stereoisomers, tautomers, polymorphs, metal complexes or N-oxides thereof.

In one embodiment, the present invention provides compounds of formula (I), wherein, C₂-C₆ heterocyclyl of the R¹ group is preferably selected from partially/fully saturated or unsaturated furyl, thienyl, pyrrolyl, isoxazolyl, thiazolyl, isothiazolyl, thiadiazolyl, pyrazolyl, oxazolyl, imidazolyl, oxadiazolyl, triazolyl, pyridinyl, pyridazinyl, pyrimidinyl or pyrazinyl.

In one embodiment, the present invention provides compounds of formula (I), wherein, C₂-C₆ heterocyclyl of the R⁵ group is preferably selected from partially/fully saturated or unsaturated furyl, thienyl, pyrrolyl, isoxazolyl, thiazolyl, isothiazolyl, thiadiazolyl, pyrazolyl, oxazolyl, imidazolyl, oxadiazolyl, triazolyl, pyridinyl, pyridazinyl, pyrimidinyl or pyrazinyl.

In one embodiment, the present invention provides compounds of formula (I), wherein, C₂-C₆ heterocyclyl of the R⁶ group is preferably selected from oxetanyl, partially/fully saturated or unsaturated furyl, thienyl, pyrrolyl, isoxazolyl, thiazolyl, isothiazolyl, thiadiazolyl, pyrazolyl, oxazolyl, imidazolyl, oxadiazolyl, triazolyl, pyridinyl, pyridazinyl, pyrimidinyl or, pyrazinyl.

In one embodiment, the present invention provides compounds of formula (I), wherein, C₂-C₆ heterocyclyl of the R group is preferably selected from partially/fully saturated or unsaturated furyl, thienyl, pyrrolyl, isoxazolyl, thiazolyl, isothiazolyl, thiadiazolyl, pyrazolyl, oxazolyl, imidazolyl, oxadiazolyl, triazolyl, pyridinyl, pyridazinyl, pyrimidinyl or pyrazinyl.

In one embodiment, the present invention provides compounds of formula (I), wherein A₁, A₂, A₃, A₄, and A₅ are independently C.

In another embodiment, the present invention provides compounds of formula (I), wherein A₁, A₂, A₃ and A₅ are independently C, and A₄ is N.

In yet another embodiment, the present invention provides compounds of formula (I), wherein A₁, A₂, A₃ and A₄ are independently C, and A₅ is N.

In yet another embodiment, the present invention provides compounds of formula (I), wherein A₁, A₂, A₄ and A₅ are independently C, and A₃ is N.

In yet another embodiment, the present invention provides compounds of formula (I), wherein A₂, A₃, A₄ and A₅ are independently C, and A₁ is N.

In yet another embodiment, the present invention provides compounds of formula (I), wherein A₁, A₃, A₄ and A₅ are independently C, and A₂ is N.

In another embodiment, the present invention provides compounds of formula (I), wherein A₁ and A₃ are independently N, A₂, A₄, and A₅ is independently C.

In yet another embodiment, the present invention provides compounds of formula (I), wherein A₂ and A₃ are independently N, A₁, A₄, and A₅ is independently C.

In yet another embodiment, the present invention provides compounds of formula (I), wherein A₂ and A₄ are independently N, A₁, A₃, and A₅ is independently C.

In yet another embodiment, the present invention provides compounds of formula (I), wherein A₁, A₃ and A₄ are independently N, and A₂ and A₅ are independently C.

In yet another embodiment, the present invention provides compounds of formula (I), wherein A₂, A₃ and A₄ are independently N, and A₁ and A₅ are independently C.

In a preferred embodiment, the present invention provides compounds of formula (I), wherein A₁, A₂, A₃, A₄ and A₅ are independently C.

In another preferred embodiment, the present invention provides compounds of formula (I), wherein A₁, A₂, A₃, and A₅ are independently C and A₄ is N.

In yet another preferred embodiment, the present invention provides compounds of formula (I), wherein A₁, A₂, A₄, and A₅ are independently C and A₃ is N.

In yet another preferred embodiment, the present invention provides compounds of formula (I), wherein A₁, A₂, and A₅ are independently C; A₃ and A₄ are independently N.

In yet another preferred embodiment, the present invention provides compounds of formula (I), wherein A₂, A₃, A₄, and A₅ are independently C and A₁ is N.

In yet another preferred embodiment, the present invention provides compounds of formula (I), wherein A₂, A₃, and A₅ are independently C; A₁ and A₄ are independently N.

In one embodiment, the present invention provides compounds of formula (I), wherein D is D₁, and Z is a direct bond.

In another embodiment, the present invention provides compounds of formula (I), wherein D is D₁, and Z is —C(═O)—.

In one embodiment, the present invention provides compounds of formula (I), wherein D is D₂, and Z is a direct bond.

In a one embodiment, the present invention provides compounds of formula (I), wherein D is D₃, and Z is a direct bond.

In one embodiment, the present invention provides compounds of formula (I), wherein D is D₁ or D₂, and Y is O.

In another embodiment, the present invention provides compounds of formula (I), wherein D is D₁ or D₂, and Y is NR.

In a preferred embodiment, the present invention provides compounds of formula (I), wherein D is D₁ or D₂, and Y is O.

In an embodiment, the present invention provides compounds of formula (I), wherein R₁ is C₁-C₆ alkyl.

In another embodiment, the present invention provides compounds of formula (I), wherein R₁ is C₁-C₆ alkyl substituted by optionally substituted phenyl; wherein said optionally substitution is selected from halogen, CN, methyl or methoxy.

In an embodiment, the present invention provides compounds of formula (I), wherein R² is C₁-C₆ alkyl.

In a preferred embodiment, the present invention provides compounds of formula (I), wherein R² is methyl or ethyl.

In an embodiment, the present invention provides compounds of formula (I), wherein R³ is C₁-C₆ alkyl.

In another embodiment, the present invention provides compounds of formula (I), wherein R³ is C₁-C₆ haloalkyl.

In yet another embodiment, the present invention provides compounds of formula (I), wherein R³ is halogen.

In yet another embodiment, the present invention provides compounds of formula (I), wherein R³ is C₁-C₆ alkoxy.

In a preferred embodiment, the present invention provides compounds of formula (I), wherein R³ is CF₃.

In another preferred embodiment, the present invention provides compounds of formula (I), wherein R³ is fluoro or chloro.

In a preferred embodiment, the present invention provides compounds of formula (I), wherein R⁴ is C₁-C₆ alkyl.

In a preferred embodiment, the present invention provides compounds of formula (I), wherein R⁴ is C₁-C₆ alkyl optionally substituted with R^{a} wherein R^{a} is selected from F, CN, methyl, ethyl, (n, iso) propyl, methoxy, CF₃, cyclopropyl, C₂-C₆ heterocyclyl; In a preferred embodiment, the present invention provides compounds of formula (I), wherein R⁵ is C₁-C₆ alkyl.

In a preferred embodiment, the present invention provides compounds of formula (I), wherein R⁵ is C₁-C₆ alkyl optionally substituted with R^{a} wherein R^{a} is selected from F, CN, methyl, ethyl, (n, iso) propyl, methoxy, CF₃, cyclopropyl, C₂-C₆ heterocyclyl; In a preferred embodiment, the present invention provides compounds of formula (I), wherein R⁶ is C₁-C₆ alkyl.

In a preferred embodiment, the present invention provides compounds of formula (I), wherein R⁶ is C₁-C₆ alkyl optionally substituted with R^{a} wherein R^{a} is selected from F, CN, methyl, ethyl, (n, iso) propyl, methoxy, CF₃, cyclopropyl, C₂-C₆ heterocyclyl.

In a preferred embodiment, the present invention provides compounds of formula (I), wherein R⁷ is C₁-C₆ alkyl, —COR⁵ and C₂-C₆ heterocyclyl.

In an embodiment R⁸ and R^{8a} are independently selected from the group consisting of hydrogen, halogen, cyano, C₁-C₄-alkyl, C₁-C₄-haloalkyl;

In a preferred embodiment the present invention provides compounds of formula (I), wherein R⁸ and R^{8a} are independently selected from the group consisting of hydrogen, chloro, fluoro, cyano, methyl, ethyl, isopropyl and CF₃;

In a preferred embodiment the present invention provides compounds of formula (I), wherein C₃-C₆ cycloalkyl is selected from cyclopropyl, cyclobutyl cyclopentyl or cyclohexyl. Preferably, cyclopropyl or cyclobutyl.

In a preferred embodiment, the present invention provides compounds of formula (I), wherein A₁, A₂, A₃, A₄ and A₅ are independently C; or A₁, A₂, A₃ and A₅ are independently C, and A₄ is N; or A₁, A₂, A₄ and A₅ are independently C, and A₃ is N; or A₁, A₂ and A₅ are independently C, and A₃ and A₄ are independently N; or A₂, A₃, A₄, and A₅ are independently C and A₁ is N; or A₂, A₃ and A₅ are independently C, and A₁ and A₄ are independently N.

The present invention also relates to a method for preparing compounds of formula (I).

Compounds of the present invention can exist as one or more stereoisomers. The various stereoisomers include enantiomers, diastereomers, atropisomers and geometric isomers. One skilled in the art will appreciate that one stereoisomer may be more active and/or may exhibit more beneficial effects when enriched relative to the other stereoisomer(s) or when separated from the other stereoisomer(s). Additionally, the skilled artisan knows how to separate, enrich, and/or to selectively prepare said stereoisomers. The compounds of the present invention may be present as a mixture of stereoisomers, individual stereoisomers or as an optically active form.

In case the compounds of formula (I) are appearing as the cationic part of a salt or are capable of forming such a cationic part of a salt, the respective anionic part can be inorganic or organic. Alternatively, in case the compounds of formula (I) are appearing as the anionic part of a salt or are capable of forming such an anionic part of a salt, the respective cationic part can be inorganic or organic. Examples of the inorganic anion part of the salt include but are not limited to chloride, bromide, iodide, fluoride, sulfate, phosphate, nitrate, nitrite, hydrogen carbonates, and hydrogen sulfate. Examples of the organic anion part of the salt include but are not limited to formate, alkanoates, carbonates, acetates, trifluoroacetate, trichloroacetate, propionate, glycolate, thiocyanate, lactate, succinate, malate, citrates, benzoates, cinnamates, oxalates, alkylsulphates, alkylsulphonates, arylsulphonates aryldisulphonates, alkyl phosphonates, aryl phosphonates, aryl diphosphonates, p-toluenesulphonate, and salicylate. Examples of the inorganic cation part of the salt include but are not limited to alkali and alkaline earth metals. Examples of the organic cation part of the salt include but are not limited to cations derived from pyridine, methyl amine, imidazole, benzimidazole, histidine, phosphazene, tetramethyl ammonium, tetrabutylammonium, choline and trimethylamine.

Metal ions in the metal complexes of the compounds of formula (I) are especially the ions of the elements of the second main group, especially calcium and magnesium, of the third and fourth main groups, especially aluminum, tin and lead, and also of the first to eighth transition groups, especially chromium, manganese, iron, cobalt, nickel, copper, zinc and others. Particular preference is given to the metal ions of the elements of the fourth period and the first to eighth transition groups. Here, the metals can be present in the various valencies that they can assume.

Another embodiment of the present invention discloses the compound of formula (I), salts, metal complexes, stereoisomers, diastereoisomers, enantiomers, chiral isomers, atropisomers, conformers, rotamers, tautomers, optical isomers, polymorphs, geometric isomers, or N-oxides thereof and compositions with the excipient, inert carrier or any other essential ingredients such as surfactants, additives, solid diluents and liquid diluents.

Compounds selected from formula (I), (including all stereoisomers, N-oxides, and salts thereof), typically exist in more than one form, and formula (I) thus includes all crystalline and non-crystalline forms of the compounds that formula (I) represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due to the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by formula (I) can exhibit beneficial effects (e.g., suitability for preparation of useful formulations, improved biological performance) relative to another polymorph or a mixture of polymorphs of the same compound represented by formula (I). Preparation and isolation of a particular polymorph of a compound represented by formula (I) can be achieved by methods known to those skilled in the art including, for example, crystallization using selected solvents and temperatures.

The present invention also relates to a composition for controlling or preventing insect and mite pests. The said composition comprises a biologically effective amount of the compound of formula (I) and at least one additional component selected from the group consisting of surfactants and auxiliaries.

Another embodiment of the present invention relates to a compound of formula (I) or its N-oxides or salts into customary types of agrochemical compositions, e. g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further types of compositions are defined in the “Catalogue of pesticide formulation types and international coding system”, Technical Monograph No. 2, 6^{th} Ed. May 2008, CropLife International.

The above-mentioned compositions are prepared in a known manner, such as described by Mollet and Grubemann, “Formulation Technology”, Wiley VCH, Weinheim, 2001; or Knowles, “New Developments in Crop Protection Product Formulation”, Agrow Reports DS243, T&F Informa, London, 2005. Examples for suitable auxiliaries for formulations and/or agrochemical compositions according to the invention are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers in this context are for instance water and organic solvents, such as mineral oil fractions of medium to high boiling point, e. g. kerosene, diesel oil, oils of vegetable or animal origin; aliphatic, cyclic and aromatic hydrocarbons, e. g. toluene, paraffin, tetrahydronaphthalene, alkylated naphthalenes; alcohols, e. g. ethanol, propanol, butanol, benzyl alcohol, cyclohexanol; glycols; dimethyl sulfoxide; ketones, e. g. cyclohexanone; esters, e. g. lactates, carbonates, fatty acid esters, gamma-butyrolactone; fatty acids; phosphonates; amines; amides, e. g. N-methyl pyrrolidone, fatty acid dimethyl amides; and mixtures thereof. Suitable solid carriers or fillers are for instance mineral earths, e. g. silicates, silica gels, talc, kaolins, limestone, lime, chalk, clays, dolomite, diatomaceous earth, bentonite, calcium sulphate, magnesium sulphate, magnesium oxide; polysaccharides, e. g. cellulose, starch; fertilizers, e. g. ammonium sulphate, ammonium phosphate, ammonium nitrate, ureas; products of vegetable origin, e. g. cereal meal, tree bark meal, wood meal, nutshell meal, and mixtures thereof.

Suitable surfactants are surface-active compounds, for instance such as anionic, cationic, nonionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol. 1: “Emulsifiers & Detergents”, McCutcheon's Directories, Glen Rock, USA, 2008 (International Ed. or North American Ed.).

Suitable anionic surfactants are for instance alkali, alkaline earth or ammonium salts of sulfonates, sulphates, phosphates, carboxylates, and mixtures thereof. Examples of sulfonates are alkylaryl sulfonates, diphenyl sulfonates, alpha-olefin sulfonates, lignin sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkyl naphthalenes, sulfosuccinates or sulfosuccinamates. Examples of sulphates are sulphates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are for instance alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide.

Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are homo- or copolymers of vinyl pyrrolidone, vinyl alcohols, or vinyl acetate.

Suitable cationic surfactants are for instance quaternary surfactants, for example quaternary ammonium compounds with one or two hydrophobic groups, or salts of long-chain primary amines. Suitable amphoteric surfactants are alkylbetains and imidazolines. Suitable block polymers are for instance block polymers of the A-B or A-B-A type comprising blocks of polyethylene oxide and polypropylene oxide, or of the A-B-C type comprising alkanol, polyethylene oxide and polypropylene oxide. Suitable polyelectrolytes are for instance polyacids or polybases. Examples of polyacids are alkali salts of polyacrylic acid or polyacid comb polymers. Examples of polybases are polyvinyl amines or polyethylene amines.

Suitable adjuvants are compounds, which have a negligible or even no pesticidal activity themselves, and which improve the biological performance of the compound of formula (I) on the target. Examples are surfactants, mineral or vegetable oils, and other auxiliaries. Further examples are listed by Knowles, “Adjuvants and Additives”, Agrow Reports DS256, T&F Informa UK, 2006, chapter 5.

Suitable thickeners are for instance polysaccharides (e. g. xanthan gum, carboxymethyl cellulose), inorganic clays (organically modified or unmodified), polycarboxylates, and silicates.

Suitable bactericides are for instance bronopol and isothiazolinone derivatives such as alkylisothiazolinones and benzisothiazolinones.

Suitable anti-freezing agents are for instance ethylene glycol, propylene glycol, urea and glycerin.

Suitable anti-foaming agents are for instance silicones, long chain alcohols, and salts of fatty acids.

Suitable colorants (e. g. in red, blue, or green) are for instance pigments of low water solubility and water-soluble dyes. Examples are inorganic colorants (e. g. iron oxide, titan oxide, iron hexacyanoferrate) and organic colorants (e. g. alizarin-, azo- and phthalocyanine colorants).

Suitable tackifiers or binders are for instance polyvinyl pyrrolidones, polyvinyl acetates, polyvinyl alcohols, polyacrylates, biological or synthetic waxes, and cellulose ethers.

Examples for composition types and their preparation are:

### I) Water-Soluble Concentrates (SL, LS)

10-60 wt % of a compound of formula (I) and 5-15 wt % wetting agent (e. g. alcohol alkoxylates) are dissolved in water and/or in a water-soluble solvent (e. g. alcohols) ad 100 wt %. The active substance dissolves upon dilution with water.

### ii) Dispersible Concentrates (DC)

5-25 wt % of a compound of formula (I) and 1-10 wt % dispersant (e. g. polyvinyl pyrrolidone) are dissolved in an organic solvent (e. g. cyclohexanone) ad 100 wt %. Dilution with water gives a dispersion of the active substance.

iii) Emulsifiable Concentrates (EC)

15-70 wt % of a compound of formula (I) and 5-10 wt % emulsifiers (e. g. calcium dodecylbenzenesulfonate and castor oil ethoxylate) are dissolved in a water-insoluble organic solvent (e. g. aromatic hydrocarbon) ad 100 wt %. Dilution with water gives an emulsion of the active substance.

### iv) Emulsions (EW, EO, ES)

5-40 wt % of a compound of formula (I) and 1-10 wt % emulsifiers (e. g. calcium dodecylbenzenesulfonate and castor oil ethoxylate) are dissolved in a 20-40 wt % water-insoluble organic solvent (e. g. aromatic hydrocarbon). This mixture is introduced into water ad 100 wt % by means of an emulsifying machine and made into a homogeneous emulsion. Dilution with water gives an emulsion of the active substance.

### v) Suspensions (SC, OD, FS)

In an agitated ball mill, 20-60 wt % of a compound of formula (I) are comminuted with the addition of 2-10 wt % dispersants and wetting agents (e. g. sodium lignosulfonate and alcohol ethoxylate), 0.1-2 wt % thickener (e. g. xanthan gum) and water ad 100 wt % to give a fine active substance suspension. Dilution with water gives a stable suspension of the active substance. For FS type composition up to 40 wt % binder (e. g. polyvinyl alcohol) is added.

### vi) Water-Dispersible Granules and Water-Soluble Granules (WG, SG)

50-80 wt % of a compound of formula (I) are ground finely with the addition of dispersants and wetting agents (e. g. sodium lignosulfonate and alcohol ethoxylate) ad 100 wt % and prepared as water-dispersible or water-soluble granules by means of technical appliances (e. g. extrusion, spray tower, fluidized bed). Dilution with water gives a stable dispersion or solution of the active substance.

vii) Water-Dispersible Powders and Water-Soluble Powders (WP, SP, WS)

50-80 wt % of a compound of formula (I) are ground in a rotor-stator mill with addition of 1-5 wt % dispersants (e. g. sodium lignosulfonate), 1-3 wt % wetting agents (e. g. alcohol ethoxylate) and a solid carrier (e. g. silica gel) ad 100 wt %. Dilution with water gives a stable dispersion or solution of the active substance.

viii) Gel (GW, GF)
In an agitated ball mill, 5-25 wt % of a compound of formula (I) are comminuted with the addition of 3-10 wt % dispersants (e. g. sodium lignosulfonate), 1-5 wt % thickener (e. g. carboxymethyl cellulose) and water ad 100 wt % to give a fine suspension of the active substance. Dilution with water gives a stable suspension of the active substance.

### ix) Microemulsion (ME)

5-20 wt % of a compound of formula (I) are added to a 5-30 wt % organic solvent blend (e. g. fatty acid dimethyl amide and cyclohexanone), a 10-25 wt % surfactant blend (e. g. alcohol ethoxylate and arylphenol ethoxylate), and water ad 100%. This mixture is stirred for 1 h to produce spontaneously a thermodynamically stable microemulsion of the active substance.

### x) Microcapsules (CS)

An oil phase comprising 5-50 wt % of a compound of formula (I), 0-40 wt % of a water insoluble organic solvent (e. g. aromatic hydrocarbon), 2-15 wt % of acrylic monomers (e. g. methylmethacrylate, methacrylic acid and a di- or triacrylate) are dispersed into an aqueous solution of a protective colloid (e. g. polyvinyl alcohol). Radical polymerization results in the formation of poly(meth)acrylate microcapsules. Alternatively, an oil phase comprising 5-50 wt % of a compound of formula (I) according to the invention, 0-40 wt % of a water insoluble organic solvent (e. g. aromatic hydrocarbon), and an isocyanate monomer (e. g. diphenylmethene-4,4′-diisocyanatae) are dispersed into an aqueous solution of a protective colloid (e. g. polyvinyl alcohol). The addition of a polyamine (e. g. hexamethylenediamine) results in the formation of polyurea microcapsules comprising the active substance. The monomers amount to 1-10 wt %. The wt % relates to the total CS composition.

### xi) Dustable Powders (DP, DS)

1-10 wt % of a compound of formula (I) are ground finely and mixed intimately with a solid carrier (e. g. finely divided kaolin) ad 100 wt %.

xii) Granules (GR, FG)

0.5-30 wt % of a compound of formula (I) are ground finely and associated with a solid carrier (e. g. silicate) ad 100 wt %. Granulation is achieved by extrusion, spray-drying or fluidized bed.

xiii) Ultra-Low Volume Liquids (UL)

1-50 wt % of a compound of formula (I) are dissolved in an organic solvent (e. g. aromatic hydrocarbon) ad 100 wt %.

The composition types i) to xiii) may optionally comprise further auxiliaries, such as 0.1-1 wt % bactericides, 5-15 wt % anti-freezing agents, 0.1-1 wt % anti-foaming agents, and 0.1-1 wt % colorants.

Another embodiment of the present invention provides agrochemical compositions containing a compound of formula (I), which comprise the active substance between 0.01 and 95% by weight, preferably between 0.1 and 90%, and more preferably between 1 and 70%, in particular between 10 and 60% by weight of the active substance. The active substances are employed in a purity of from 90% to 100%, preferably from 95% to 100% (according to NMR spectrum).

Water-soluble concentrates (LS), suspoemulsions (SE), flowable concentrates (FS), powders for dry treatment (DS), water-dispersible powders for slurry treatment (WS), water-soluble powders (SS), emulsions (ES), emulsifiable concentrates (EC) and gels (GF) are usually employed for the purposes of the treatment of plant propagation materials, particularly seeds. The compositions in question give, after two-to-tenfold dilution, active substance concentrations of from 0.01 to 60% by weight, preferably from 0.1 to 40% by weight in the ready-to-use preparations. Application can be carried out before or during sowing.

Methods for applying compounds of formula (I) and compositions thereof, respectively, on to plant propagation material, especially seeds, include dressing, coating, pelleting, dusting, soaking and in-furrow application methods of the propagation material. Preferably, compounds of formula (I) or the compositions thereof, respectively, are applied on to the plant propagation material in a way such that the germination is not induced, e. g. by seed dressing, pelleting, coating and dusting.

When employed in plant protection, the amounts of active substances applied are, depending on the kind of effect desired, ranging from 0.001 to 2 kg per ha, preferably from 0.005 to 2 kg per ha, more preferably from 0.05 to 0.9 kg per ha, in particular from 0.1 to 0.75 kg per ha.

For the treatment of plant propagation materials such as seeds, e. g. by dusting, coating or drenching seed, amounts of the active substance ranging from 0.1 to 1000 g, preferably from 1 to 1000 g, more preferably from 1 to 100 g and most preferably from 5 to 100 g, per 100 kilogram of plant propagation material (preferably seed) are generally required.

When used in the protection of materials or stored products, the amount of active substance applied depends on the kind of application area and on the desired effect. Amounts customarily applied in the protection of materials are ranging from 0.001 g to 2 kg, preferably from 0.005 g to 1 kg of active substance per cubic meter of treated material.

Various types of oils, wetters, adjuvants, fertilizer, or micronutrients, and other pesticides (e.g. herbicides, insecticides, fungicides, growth regulators, safeners) may be added to the active substances or the compositions comprising them as premix or, if appropriate, not until immediately prior to use (tank mix). These agents can be admixed with the compositions according to the present invention in a weight ratio of 1:100 to 100:1, preferably 1:10 to 10:1.

The user can apply the compositions according to the present invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the present invention is obtained in this way. Usually, 20 to 2000 liters, preferably 50 to 400 liters, of the ready-to-use spray liquor are applied per hectare of agricultural useful area.

According to one embodiment, individual components of the composition according to the present invention such as parts of a kit or parts of a binary or ternary mixture may be mixed by the user himself in a spray tank and further auxiliaries may be used in addition, if appropriate.

The compounds and compositions of the present invention are in this way agronomically useful for protecting field crops from phytophagenic invertebrate pests, and also nonagronomically for protecting other horticultural crops and plants from phytophagenic invertebrate pests. This utility includes protecting crops and other plants (i.e. both agronomically and nonagronomically) that contain genetic material introduced by genetic engineering (i.e. transgenic) or modified by mutagenesis to provide advantageous traits.

Compounds of the present invention are characterized by favorable metabolic and/or soil residual patterns and exhibit activity controlling a spectrum of agronomic and non-agronomic invertebrate pests.

The compounds of the present invention are preventively and/or curatively valuable active ingredients in the field of pest control, even at low rates of application, which can be used against pesticide resistant pests such as insects, and/or have a very favorable biocidal spectrum and are well tolerated by warm-blooded species, fish and plants.

Compounds of the present invention display activity against economically important agronomic, forest, greenhouse, nursery, ornamentals, food and fiber, public and animal health, domestic and commercial structure, household and stored product pests. These include larvae of the order Lepidoptera, such as armyworms, cutworms, loopers, and heliothines in the family Noctuidae (e.g., fall armyworm (Spodoptera fugiperda J. E. Smith), beet armyworm (Spodoptera exigua Hubner), black cutworm (Agrotis ipsilon Hufnagel), cabbage looper (Trichoplusia ni Hubner), tobacco budworm (Heliothis virescens Fabricius)); borers, casebearers, webworms, coneworms, cabbageworms and skeletonizers from the family Pyralidae (e.g., European corn borer (Ostrinia nubilalis Hubner), navel orangeworm (Amyelois transitella Walker), corn root webworm (Crambus caliginosellus Clemens), sod webworm (Herpetogramma licarsisalis Walker)); leafrohers, budworms, seed worms, and fruit worms in the family Tortricidae (e.g., codling moth (Cydia pomonella Linnaeus), grape berry moth (Endopiza viteana Clemens), oriental fruit moth (Grapholita molesta Busck)); and many other economically important lepidoptera (e.g., diamondback moth (Plutella xylostella Linnaeus), pink bollworm (Pectinophora gossypiella Saunders), gypsy moth (Lymantria dispar Linnaeus)); nymphs and adults of the order Blattodea including cockroaches from the families Blattellidae and Blattidae (e.g., oriental cockroach (Blatta orientalis Linnaeus), Asian cockroach (Blatella asahinai Mizukubo), German cockroach (Blattella gemnanica Linnaeus), brownbanded cockroach (Supella longipalpa Fabricius), American cockroach (Periplaneta americana Linnaeus), brown cockroach (Periplaneta brunnea Burmeister), Madeira cockroach (Leucophaea maderae Fabricius)); foliar feeding larvae and adults of the order Coleoptera including weevils from the families Anthribidae, Bruchidae, and Curculionidae (e.g., boll weevil (Anthonomus grandis Boheman), rice water weevil (Lissorhoptrus oryzophilus Kuschel), granary weevil (Sitophilus granarius Linnaeus), rice weevil (Sitophilus oryzae Linnaeus)); flea beetles, cucumber beetles, rootworms, leaf beetles, potato beetles, and leafminers in the family Chrysomelidae (e.g., Colorado potato beetle (Leptinotarsa decemLineata Say), western corn rootworm (Diabrotica virgifera LeConte)); chafers and other beetles from the family Scaribaeidae (e.g., Japanese beetle (Popilia japonica Newman) and European chafer (Rhizotrogus majalis Razoumowsky)); carpet beetles from the family Dermestidae; wireworms from the family Elateridae; bark beetles from the family Scolytidae and flour beetles from the family Tenebrionidae. In addition it includes: adults and larvae of the order Dermaptera including earwigs from the family Forficulidae (e.g., European earwig (Forficula auricularia Linnaeus), black earwig (Chelisoches mono Fabricius)); adults and nymphs of the orders Hemiptera and Homoptera such as, plant bugs from the family Miridae, cicadas from the family Cicadidae, leafhoppers (e.g. Empoasca spp.) from the family Cicadellidae, planthoppers from the families Fulgoroidae and Delphacidae, treehoppers from the family Membracidae, psyllids from the family Psyllidae, whiteflies from the family Aleyrodidae, aphids from the family Aphididae, phylloxera from the family Phylloxeridae, mealybugs from the family Pseudococcidae, scales from the families Coccidae, Diaspididae and Margarodidae, lace bugs from the family Tingidae, stink bugs from the family Pentatomidae, cinch bugs (e.g., Blissus spp.) and other seed bugs from the family Lygaeidae, spittlebugs from the family Cercopidae squash bugs from the family Coreidae, and red bugs and cotton stainers from the family Pyrrhocoridae. Also included are adults and larvae of the order Acari (mites) such as spider mites and red mites in the family Tetranychidae (e.g., European red mite (Panonychus ulmi Koch), two spotted spider mite (Tetranychus urticae Koch), McDaniel mite (Tetranychus mcdanieli McGregor)), flat mites in the family Tenuipalpidae (e.g., citrus flat mite (Brevipalpus lewisi McGregor)), rust and bud mites in the family Eriophyidae and other foliar feeding mites and mites important in human and animal health, i.e. dust mites in the family Epidermoptidae, follicle mites in the family Demodicidae, grain mites in the family Glycyphagidae, ticks in the order Ixodidae (e.g., deer tick (Ixodes scapularis Say), Australian paralysis tick (Ixodes holocyclus Neumann), American dog tick (Dermacentor variabilis Say), lone star tick (Amblyomma americanum Linnaeus) and scab and itch mites in the families Psoroptidae, Pyemotidae, and Sarcoptidae; adults and immatures of the order Orthoptera including grasshoppers, locusts and crickets (e.g., migratory grasshoppers (e.g., Melanoplus sanguinipes Fabricius, M. differentialis Thomas), American grasshoppers (e.g., Schistocerca americana Drury), desert locust (Schistocerca gregaria Forskal), migratory locust (Locusta migratoria Linnaeus), house cricket (Acheta domesticus Linnaeus), mole crickets (Gryllotalpa spp.)); adults and immatures of the order Diptera including leafminers, midges, fruit flies (Tephritidae), fruit flies (e.g., Oscinella frit Linnaeus), soil maggots, house flies (e.g., Musca domestica Linnaeus), lesser house flies (e.g., Fannia canicularis Linnaeus, F. femoralis Stein), stable flies (e.g., Stomoxys calcitrans Linnaeus), face flies, horn flies, blow flies (e.g., Chiysomya spp., Phonnia spp.), and other muscoid fly pests, horse flies (e.g., Tabanus spp.), botflies (e.g., Gastrophilus spp., Oestrus spp.), cattle grubs (e.g., Hypoderma spp.), deer flies (e.g., Chrysops spp.), keds (e.g., Melophagus ovinus Linnaeus) and other Brachycera, mosquitoes (e.g., Aedes spp., Anopheles spp., Culex spp.), black flies (e.g., Prosimulium spp., Simulium spp.), biting midges, sand flies, sciarids, and other Nematocera; adults and immatures of the order Thysanoptera including onion thrips (Thrips tabaci Lindeman) and other foliar feeding thrips; insect pests of the order Hymenoptera including ants (e.g., red carpenter ant (Camponotus ferrugineus Fabricius), black carpenter ant (Camponotus pennsylvanicus De Geer), Pharaoh ant (Monomorium pharaonis Linnaeus), little fire ant (Wasmannia auropunctata Roger), fire ant (Solenopsis geminata Fabricius), red imported fire ant (Solenopsis invicta Buren), Argentine ant (Iridomyrmex humilis Mayr), crazy ant (Paratrechina longicornis Latreille), pavement ant (Tetramorium caespitum Linnaeus), cornfield ant (Lasius alienus Förster), odorous house ant (Tapinoma sessile Say)), bees (including carpenter bees), hornets, yellow jackets and wasps; insect pests of the order Isoptera including the eastern subterranean termite (Reticulitermes flavipes Kollar), western subterranean termite (Reticulitermes hesperus Banks), Formosan subterranean termite (Coptotermes formosanus Shiraki), West Indian drywood termite (Incisitermes immigrans Snyder) and other termites of economic importance; insect pests of the order Thysanura such as silverfish (Lepisma saccharina Linnaeus) and firebrat (Thermobia domestica Packard); insect pests of the order Mallophaga and including the head louse (Pediculus humanus capitis De Geer), body louse (Pediculus humanus Linnaeus), chicken body louse (Menacanthus stramineus Nitszch), dog biting louse (Trichodectes cams De Geer), fluff louse (Goniocotes gallinae De Geer), sheep body louse (Bovicola ovis Schrank), short-nosed cattle louse (Haematopinus eurystemus Nitzsch), long-nosed cattle louse (Linognathus vituli Linnaeus) and other sucking and chewing parasitic lice that attack man and animals; insect pests of the order Siphonoptera including the oriental rat flea (Xenopsylla cheopis Rothschild), cat flea (Ctenocephalides felis Bouche), dog flea (Ctenocephatides canis Curtis), hen flea (Ceratophyllus gallinae Schrank), sticktight flea (Echidnophaga gallinacea Westwood), human flea (Pulex irritans Linnaeus) and other fleas afflicting mammals and birds. Additional arthropod pests covered include: spiders in the order Araneae such as the brown recluse spider (Loxosceles reclusa Gertsch and Mulaik) and the black widow spider (Latrodectus mactans Fabricius), and centipedes in the order Scutigeromorpha such as the house centipede (Scutigera coleoptrata Linnaeus). Activity also includes members of the Classes Nematoda, Cestoda, Trematoda, and Acanthocephala including economically important members of the orders Strongylida, Ascaridida, Oxyurida, Rhabditida, Spirurida, and Enoplida such as but not limited to economically important agricultural pests (i.e. root knot nematodes in the genus Meloidogyne, lesion nematodes in the genus Pratylenchus, stubby root nematodes in the genus Trichodorus, etc.) and animal and human health pests (i.e. all economically important flukes, tapeworms, and roundworms, such as Strongylus vulgaris in horses, Toxocara canis in dogs, Haemonchus contortus in sheep, Dirofilaria immitis Leidy in dogs, Anoplocephala peifoliata in horses, Fasciola hepatica Linnaeus in ruminants, etc.).

Compounds of the present invention show particularly activity against pests in the order Homoptera including: Acyrthisiplionpisum Harris (pea aphid), Aphis craccivora Koch (cowpea aphid), Aphis fabae Scopoli (black bean aphid), Aphis gossypii Glover (cotton aphid, melon aphid), Aphis pomi De Geer (apple aphid), Aphis spiraecola Patch (spirea aphid), Aulacorthum solani Kaltenbach (foxglove aphid), Chaetosiphon fragaefolii Cockerell (strawberry aphid), Diuraphis noxia Kurdjumov/Mordvilko (Russian wheat aphid), Dysaphis plantaginea Paaserini (rosy apple aphid), Eriosoma lanigerum Hausmann (woolly apple aphid), Hyalopterus pruni Geoffroy (mealy plum aphid), Lipaphis erysimi Kaltenbach (tarnip aphid), Metopolophium dirrhodum Walker (cereal aphid), Macrosipum euphorbiae Thomas (potato aphid), Myzus persicae Sulzer (peach-potato aphid, green peach aphid), Nasonovia ribisnigri Mosley (lettuce aphid), Pemphigus spp. (root aphids and gall aphids), Rhopalosiphum maidis Fitch (corn leaf aphid), Rhopalosiphum padi Linnaeus (bird cherry-oat aphid), Schizaphis graminum Rondani (greenbug), Sitobion avenae Fabricius (English grain aphid), Therioaphis maculata Buckton (spotted alfalfa aphid), Toxoptera aurantii, Boyer de Fonscolombe (black citrus aphid), and Toxoptera citiicida Kirkaldy (brown citrus aphid); Adelges spp. (adelgids); Phylloxera devastatrix Pergande (pecan phylloxera); Bemisia tabaci Gennadius (tobacco whitefly, sweetpotato whitefly), Bemisia argentifolii Bellows and Perring (silverleaf whitefly), Dialeurodes citri Ashmead (citrus whitefly) and Trialeurodes vaporariorum Westwood (greenhouse whitefly); Empoasca fabae Harris, (potato leafhopper), Laodelphax striatellus Fallen (smaller brown planthopper), Macrolestes quadrilineatus Forbes (aster leafhopper), Nephotettix cinticeps Uhler (green leafhopper), Nephotettix nigropictus Stal (rice leafhopper), Nilaparvata lugens Stal (brown planthopper), Peregrinus maidis Ashmead (corn planthopper), Sogatellafurcifera Horvath (white-backed planthopper), Sogatodes orizicola Muir (rice delphacid), Typhlocyba pomaria McAfee white apple leafhopper, Erythroneoura spp. (grape leafhoppers); Magicidada septendecim Linnaeus (periodical cicada); cerya purchasi Maskell (cottony cushion scale), Quadraspidiotus perniciosus Comstock (San Jose scale); Planococcus citri Risso (citrus mealybug); Pseudococcus spp. (other mealybug complex); Cacopsylla pyricola Foerster (pear psylla), Trioza diospyri Ashmead (persimmon psylla). Compounds of the present invention also have commercially significant activity on members from the order of Lepidoptera (e.g., Alabama argillacea Hubner (cotton leaf worm), Archips argyrospila Walker (fruit tree leaf roller), A. rosana Linnaeus (European leaf roller) and other Archips species, Chilo suppressalis Walker (rice stem borer), Cnaphalocrosis medinalis Guenee (rice leaf roller), Crambus caliginosellus Clemens (corn root webworm), Crambus teterrellus Zincken (bluegrass webworm), Cydia pomonella Linnaeus (codling moth), Earias insulana Boisduval (spiny bollworm), Earias vittella Fabricius (spotted bollworm), Helicoveipa armigera Hνbner (American bollworm), Helicoverpa zea Boddie (corn earworm), Heliothis virescens Fabricius (tobacco budworm), Herpetogramma licarsisalis Walker (sod webworm), Lobesia botrana Denis and Schiffeπnνller (grape berry moth), Pectinophora gossypiella Saunders (pink bollworm), Phyllocnistis citrella Stainton (citrus leafminer), Pieris brassicae Linnaeus (large white butterfly), Pieris rapae Linnaeus (small white butterfly), Plutella xylostella Linnaeus (diamondback moth), Spodoptera exigua Hubner (beet armyworm), Spodoptera litura Fabricius (tobacco cutworm, cluster caterpillar), Spodoptera frugiperda J. E. Smith (fall armyworm), Trichoplusia ni Hνbner (cabbage looper) and Tula absoluta Meyrick (tomato leafminer)).

The compounds also have activity on members from the order Hemiptera including: Acrostemum hilare Say (green stink bug), Anasa tristis De Geer (squash bug), Blissus leucopterus Say (chinch bug), Corythuca gossypii Fabricius (cotton lace bug), Cyrtopeltis modesta Distant (tomato bug), Dysdercus suturellus Herrich-S chaffer (cotton stainer), Euchistus servus Say (brown stink bug), Euchistus variolrius Palisot deBeauvois (one-spotted stink bug), Graptósthetus spp. (complex of seed bugs), Leptoglossus corculus Say (leaf-footed pine seed bug), Lygus lineolaris Palisot de Beauvois (tarnished plant bug), Nezara viridula Linnaeus (southern green stink bug), Oebalus pugnax Fabricius (rice stink bug), Oncopeltus fasciatus DaEas (large milkweed bug), Pseudatomoscelis seriatus Reuter (cotton fleahopper).

Other insect orders controlled by compounds of the present invention include: Thysanoptera (e.g., Frankliniella occidentalis Pergande (western flower thrip), Scirthothips citri Moulton (citrus thrip), Sericothrips variabilis Beach (soybean thrip), and Thrips tabaci Lindeman (onion thrip); and the order Coleoptera (e.g., Leptinotarsa decemLineata Say (Colorado potato beetle), Epilachna varivestis Mulsant (Mexican bean beetle) and wireworms of the genera Agriotes, Athous or Limonius).

Particularly, the compounds of formula (I), their N-oxides, their stereoisomers, their polymorphs and their salts are especially suitable for efficiently combating the following pests: Insects from the order of the lepidopterans (Lepidoptera), for example Agrotis ipsilon, Agrotis segetum, Alabama argillacea, Anticarsia gemmatalis, Argyresthia conjugella, Autographa gamma, Bupalus piniarius, Cacoecia murinana, Capua reticulana, Cheimatobia brumata, Chilo infuscatellus, Choristoneura fumiferana, Choristoneura occidentalis, Cirphis unipuncta, Cydia pomonella, Dendrolimus pini, Diaphania nitidalis, Diatraea grandiosella, Earias insulana, Earias vittella, Elasmopalpus lignosellus, Eupoecilia ambiguella, Evetria bouliana, Feltia subterranea, Galleria mellonella, Grapholita funebrana, Grapholita molesta, Helicoverpa armigera, Helicoverpa virescens, Helicoverpa zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalis, Leucinodes orbonalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scirpophaga incertulas, Scrobipalpula absoluta, Sitotroga cerealella, Sparganothis pilleriana, Spodoptera frugiperda, Spodoptera littoralis, Spodoptera litura, Spodoptera exigua, Thaumatopoea pityocampa, Tortrix viridana, Trichoplusia ni and Zeiraphera canadensis; and

Beetles (Coleoptera), for example Agrilus sinuatus, Agriotes lineatus, Agriotes obscurus, Amphimallus solstitialis, Anisandrus dispar, Anthonomus grandis, Anthonomus pomorum, Aphthona euphoridae, Athous haemorrhoidalis, Atomaria linearis, Blastophagus pimperda, Blitophaga undata, Bruchus rufimanus, Bruchus pisorum, Bruchus lentis, Byctiscus betulae, Cassida nebulosa, Cerotoma trifurcata, Cetonia aurata, Ceuthorrhynchus assimilis, Ceuthorrhynchus napi, Chaetocnema tibialis, Conoderus vespertinus, Crioceris asparagi, Ctenicera ssp., Diabrotica longicornis, Diabrotica semipunctata, Diabrotica undecimpunctata Diabrotica speciosa, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemLineata, Limonius californicus, Lissorhoptrus oryzophilus, Melanotus communis, Meligethes aeneus, Melolontha hippocastani, Melolontha, Oulema oryzae, Otiorrhynchus sulcatus, Otiorrhynchus ovatus, Phaedon cochleariae, Phyllobius pyri, Phyllotreta chrysocephala, Phyllophaga sp., Phyllopertha horticola, Phyllotreta nemorum, Phyllotreta striolata, Popillia japonica, Sitona lineatus and Sitophilus granaria; Flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Ceratitis capitata, Chrysomya bezziana, Chrysomya hominivorax, Chrysomya macellaria, Chrysops discalis, Chrysops silacea, Chrysops atlanticus, Cochliomyia hominivorax, Contarinia sorghicola Cordylobia anthropophaga, Culicoides furens, Culex pipiens, Culex nigripalpus, Culex quinquefasciatus, Culex tarsalis, Culiseta inornata, Culiseta melanura, Dacus cucurbitae, Dacus oleae, Dasineura brassicae, Delia antique, Delia coarctata, Delia platura, Delia radicum, Dermatobia hominis, Fannia canicularis, Geomyza Tripunctata, Gasterophilus intestinalis, Glossina morsitans, Glossina palpalis, Glossina fuscipes, Glossina tachinoides, Haematobia irritans, Haplodiplosis equestris, Hippelates spp., Hylemyia platura, Hypoderma lineata, Leptoconops torrens, Liriomyza sativae, Liriomyza trifolii, Lucilia caprina, Lucilia cuprina, Lucilia sericata, Lycoria pectoralis, Mansonia titillanus, Mayetiola destructor, Musca autumnalis, Musca domestica, Muscina stabulans, Oestrus ovis, Opomyza forum, Oscinella frit, Pegomya hysocyami, Phorbia antiqua, Phorbia brassicae, Phorbia coarctata, Phlebotomus argentipes, Psorophora columbiae, Psila rosae, Psorophora discolor, Prosimulium mixtum, Rhagoletis cerasi, Rhagoletis pomonella, Sarcophaga haemorrhoidalis, Sarcophaga spp., Simulium vittatum, Stomoxys calcitrans, Tabanus bovinus, Tabanus atratus, Tabanus lineola, and Tabanus similis, Tipula oleracea, and Tipula paludosa; termites (Isoptera), e.g. Calotermes flavicollis, Leucotermes flavipes, Heterotermes aureus, Reticulitermes flavipes, Reticulitermes virginicus, Reticulitermes lucifugus, Reticulitermes santonensis, Reticulitermes grassei, Termes natalensis, and Coptotermes formosanus; cockroaches (Blattaria Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis; ants, bees, wasps, sawflies (Hymenoptera), e.g. Athalia rosae, Atta cephalotes, Atta capiguara, Atta cephalotes, Atta laevigata, Atta robusta, Atta sexdens, Atta texana, Crematogaster spp., Hoplocampa minuta, Hoplocampa testudinea, Lasius niger, Monomorium pharaonis, Solenopsis geminata, Solenopsis invicta, Solenopsis richteri, Solenopsis xyloni, Pogonomyrmex barbatus, Pogonomyrmex californicus, Pheidole megacephala, Dasymutilla occidentalis, Bombus spp., Vespula squamosa, Paravespula vulgaris, Paravespula pennsylvanica, Paravespula germanica, Dolichovespula maculata, Vespa crabro, Polistes rubiginosa, Camponotus floridanus, and Linepithema humile; crickets, grasshoppers, locusts (Orthoptera), e.g. Acheta domestica, Gryllotalpa, Locusta migratoria, Melanoplus bivittatus, Melanoplus femurrubrum, Melanoplus mexicanus, Melanoplus sanguinipes, Melanoplus spretus, Nomadacris septemfasciata, Schistocerca americana, Schistocerca gregaria, Dociostaurus maroccanus, Tachycines asynamorus, Oedaleus senegalensis, Zonozerus variegatus, Hieroglyphus daganensis, Kraussaria angulifera, Calliptamus italicus, Chortoicetes terminifera, and Locustana pardalina; Araneida, e.g. Latrodectus mactans, and Loxosceles reclusa; fleas (Siphonaptera), e.g. Ctenocephalidesfelis, Ctenocephalides canis, Xenopsylla cheopis, Pulex irritans, Tunga penetrans, and Nosopsyllus fasciatus, silverfish, firebrat (Thysanura), e.g. Lepisma saccharina and Thermobia domestica, centipedes (Chilopoda), e.g. Scutigera oleoptrata, millipedes (Diplopoda), e.g. Narceus spp., Earwigs (Dermaptera), e.g. Forficula auricularia, lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon allinae, Menacanthus stramineus and Solenopotes capillatus. Collembola (springtails), e.g. Onychiurus ssp.

They are also suitable for controlling Nematodes: plant parasitic nematodes such as root knot nematodes, Meloidogyne hapla, Meloidogyne incognita, Meloidogyne javanica, and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species.

The compounds of formula (I) and their salts are also useful for controlling arachnids (Arachnoidea), such as acarians (Acarina), e.g. of the families Argasidae, Ixodidae and Sarcoptidae, such as Amblyomma americanum, Amblyomma variegatum, Argas persicus, Boophilus annulatus, Boophilus decoloratus, Boophilus microplus, Dermacentor silvarum, Hyalomma truncatum, Ixodes ricinus, Ixodes rubicundus, Ornithodorus moubata, Otobius megnini, Dermanyssus gallinae, Psoroptes ovis, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and Oligonychus pratensis.

In a further embodiment, the present invention provides the compounds of formula (I) as active and useful compounds for controlling insects selected form sucking or piercing insects such as insects from the genera Thysanoptera, Diptera and Hemiptera, in particular the following species:
Thysanoptera: Frankliniella fusca, Frankliniella occidentalis, Frankliniella tritici, Scirtothrips citri, Thrips oryzae, Thrips palmi and Thrips tabaci,
Diptera: e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Ceratitis capitata, Chrysomya bezziana, Chrysomya hominivorax, Chrysomya macellaria, Chrysops discalis, Chrysops silacea, Chrysops atlanticus, Cochliomyia hominivorax, Contarinia sorghicola Cordylobia anthropophaga, Culicoides furens, Culex pipiens, Culex nigripalpus, Culex quinquefasciatus, Culex tarsalis, Culiseta inornata, Culiseta melanura, Dacus cucurbitae, Dacus oleae, Dasineura brassicae, Delia antique, Delia coarctata, Delia platura, Delia radicum, Dermatobia hominis, Fannia canicularis, Geomyza tripunctata, Gasterophilus intestinalis, Glossina morsitans, Glossina palpalis, Glossina fuscipes, Glossina tachinoides, Haematobia irritans, Haplodiplosis equestris, Hippelates spp., Hylemyia platura, Hypoderma lineata, Leptoconops torrens, Liriomyza sativae, Liriomyza trifolii, Lucilia caprina, Lucilia cuprina, Lucilia sericata, Lycoria pectoralis, Mansonia titillanus, Mayetiola destructor, Musca autumnalis, Musca domestica, Muscina stabulans, Oestrus ovis, Opomyza florum, Oscinella frit, Pegomya hysocyami, Phorbia antiqua, Phorbia brassicae, Phorbia coarctata, Phlebotomus argentipes, Psorophora columbiae, Psila rosae, Psorophora discolor, Prosimulium mixtum, Rhagoletis cerasi, Rhagoletis pomonella, Sarcophaga haemorrhoidalis, Sarcophaga spp., Simulium vittatum, Stomoxys calcitrans, Tabanus bovinus, Tabanus atratus, Tabanus lineola, and Tabanus similis, Tipula oleracea, and Tipula paludosa;
Hemiptera, in particular aphids: Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzodes persicae, Myzus ascalonicus, Myzus cerasi, Myzus varians, Nasonovia ribis-nigri, Nilaparvata lugens, Pemphigus bursarius, Perkinsiella saccharicida, Phorodon humuli, Psylla mail, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, and Viteus vitifolii.

In one preferred embodiment, the present invention of the compounds of formula (I) are especially useful for controlling for example western flower thrips (Frankliniella occidentalis), potato leafhopper (Empoasca fabae), rice brown planthopper (Nilaparvata lugens), rice green leafhopper (Nephotettix virescens), cotton/melon aphid (Aphis gossypii), green peach aphid (Myzus persicae), tobacco/sweetpotato whitefly (Bemisia tabaci) and silverleaf whitefly (Bemisia argentifolii).

In one embodiment, the present invention further relates to a composition comprising a biologically effective amount of the compound of formula (I) and at least one additional biological active compatible compound selected from fungicides, insecticides, nematicides, acaricides, biopesticides, herbicides, plant growth regulators, antibiotics, fertilizers and nutrients. The compounds used in the composition and in combination with the compound of formula (I) are also termed as active compatible compounds.

The known and reported fungicides, insecticides, nematicides, acaricides, biopesticides, herbicides, plant growth regulators, biostimulants, antibiotics and nutrients can be combined with at least one compound of the formula (I) of the present disclosure. For example, fungicides, insecticides, nematicides, acaricides, biopesticides, herbicides, plant growth regulators, biostimulants, antibiotics, fertilizers and nutrients disclosed and reported in WO2016156129 and or WO2017153200 can be combined with at least one compound of the formula (I) of the present disclosure.

In certain instances, combinations with other invertebrate pest control compounds or agents, having a similar spectrum of control but a different mode of action, will be particularly advantageous for resistance management. Thus, compositions of the present invention can further comprise a biologically effective amount of at least one additional invertebrate pest control compound or agents having a similar spectrum of control but a different mode of action. Contacting a genetically modified plant to express a plant protection compound (e.g., protein) or the locus of the plant with a biologically effective amount of a compound of the invention can also provide a broader spectrum of plant protection and be advantageous for resistance management.

In one embodiment of the present invention, the biologically effective amount of the compound of formula (I) in the compositions ranges from 0.1 to 99% by weight with respect to the total weight of the composition, preferably from 5 to 50% by weight with respect to the total weight of the composition.

The present invention furthermore relates to a method of combating insect and mite pests, said method comprising contacting the insect and mite pests, their habit, breeding ground, food supply, plant, seed, soil, area, material or environment in which the insect and mite pests are growing or may grow, or the materials, plants, seeds, soils, surfaces or spaces to be protected from pest attack or infestation with a biologically effective amount of the compound or the composition of the present invention.

Invertebrate pests are controlled and protection of agronomic, horticultural and specialty crops, animal and human health is achieved by applying one or more of the compounds of the present invention, in an effective amount, to the environment of the pests including the agronomic and/or nonagronomic locus of infestation, to the area to be protected, or directly on the pests to be controlled. Thus, the present invention further comprises a method for the control of foliar- and soil-inhabiting invertebrates and protection of agronomic and/or nonagronomic crops, comprising contacting the invertebrates or their environment with a biologically effective amount of one or more of the compounds of the present invention, or with a composition comprising at least one such compound or a composition comprising at least one such compound and an effective amount of at least one additional biologically active compound or agent. A preferred method of contact is by spraying. Alternatively, a granular composition comprising a compound of the present invention can be applied to the plant foliage or the soil.

Compounds of the present invention are effective in delivery through plant uptake by contacting the plant with a composition comprising a compound of the present invention applied as a soil drench of a liquid formulation, a granular formulation to the soil, a nursery box treatment or a dip of transplants.

Other methods of contact include application of a compound or a composition of the present invention by direct and residual sprays, aerial sprays, seed coats, microencapsulations, systemic uptake, baits, eartags, boluses, foggers, fumigants, aerosols, dusts and many others.

The compounds of the present invention can be incorporated into baits that are consumed by the invertebrates or within devices such as traps and the like. Granules or baits comprising between 0.01-5% active ingredient, 0.05-10% moisture retaining agent(s) and 40-99% vegetable flour are effective in controlling soil insects at very low application rates, particularly at doses of active ingredient that are lethal by ingestion rather than by direct contact. The compounds of the present invention can be applied in their pure state, but most often the application will be of a formulation comprising one or more compounds with suitable carriers, diluents, and surfactants and possibly in combination with a food depending on the contemplated end use. A preferred method of application involves spraying of a water dispersion or refined oil solution of the compounds. Combinations with spray oils, spray oil concentrations, spreader, stickers, adjuvants, other solvents, and synergists such as piperonyl butoxide often enhance compound efficacy.

The rate of application required for an effective control (i.e. “biologically effective amount”) will depend on such factors as the species of invertebrate to be controlled, the pest's life cycle, life stage, its size, location, time of year, host crop or animal, feeding behavior, mating behavior, ambient moisture, temperature, and the like. Under normal circumstances, application rates of about 0.01 to 2 kg of active ingredient per hectare are sufficient to control pests in agronomic ecosystems, but as little as 0.0001 kg hectare may be sufficient or as much as 8 kg/hectare may be required. For nonagronomic applications, effective use rates will range from about 1.0 to 50 mg/square meter but as little as 0.1 mg/square meter may be sufficient or as much as 150 mg/square meter may be required. One skilled in the art can easily determine the biologically effective amount necessary for the desired level of invertebrate pest control.

The animal pest, i.e. the insects, arachnids and nematodes, the plant, soil or water in which the plant is growing can be contacted with compounds of formula (I), their N-oxides and salts or composition(s) containing them by any application method known in the art. As such, “contacting” includes both direct contact (applying the compounds/compositions directly on the animal pest or plant typically to the foliage, stem or roots of the plant) and indirect contact (applying the compounds/compositions to the locus of the animal pest or plant).

The compounds of the present invention or the pesticidal compositions comprising them may be used to protect growing plants and crops from an attack or infestation by animal pests, especially insects, acaridae or arachnids by contacting the plant/crop with a pesticidally effective amount of at least one compound of the present invention. The term “crop” refers both to growing and harvested crops.

Accordingly, the present invention relates to a method for protecting crops from an attack or infestation by insect and mite pests, which comprises contacting the crop with a biologically effective amount of the compound or the composition of the present invention, isomer, polymorph, N-oxide or salt thereof.

The compounds of the present invention are employed as such or in the form of compositions by treating the insects or the plants, plant propagation materials, such as seeds, soil, surfaces, materials or rooms to be protected from insecticidal attack with an insecticidally effective amount of the active compounds.

The application can be carried out both before and after the infection of the plants, plant propagation materials, such as seeds, soil, surfaces, materials or rooms by the insects.

Accordingly, the present invention also relates to a method for the protection of seeds from soil insects and of the seedlings roots and shoots from soil and foliar insects comprising contacting the seeds before sowing and/or after pre-germination with the compound or the composition of the present invention, N-oxide or salt thereof.

Furthermore, the present invention relates to a method for treating or protecting animals against infestation or infection by parasites which comprises orally, topically or parenterally administering or applying to the animals a biologically effective amount of compound or composition of the present invention, isomer, polymorph, N-oxide or veterinary acceptable salt thereof.

For the use in treating crop plants, the rate of application (applying effective dosages) of the compound of the present invention may be in the range of 1 gai to 5000 gai per hectare in agricultural or horticultural crops, preferably from 25 g to 600 g per hectare, more preferably from 35 g to 300 g per hectare.

The compounds and the compositions of the present invention are particularly useful in the control of a multitude of insects on various cultivated plants, such as cereals, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

Particularly, the compound or the composition of the present invention are useful in protecting agricultural crops such as cereals, corn, rice, soybean and other leguminous plants, fruits and fruit trees, grapes, nuts and nut trees, citrus and citrus trees, any horticultural plants, cucurbitaceae, oleaginous plants, tobacco, coffee, tea, cacao, sugar beet, sugar cane, cotton, potato, tomato, onions, peppers and other vegetables, and ornamentals.

The present invention still further relates to processes for preparing the compound selected from formula (I).

The compounds of the present invention are effective through both contact (via soil, glass, wall, bed net, carpet, plant parts or animal parts), and ingestion (bait or plant part).

The compounds of the present invention may also be applied against non-crop insect and mite pests, such as ants, termites, wasps, flies, mosquitos, crickets, or cockroaches. For use against said non-crop pests, compounds of the present invention are preferably used in a bait composition.

The bait can be a liquid, a solid or a semisolid preparation (e.g. a gel). Solid baits can be formed into various shapes and forms suitable to the respective application e.g. granules, blocks, sticks, disks.

Liquid baits can be filled into various devices to ensure proper application, e.g. open containers, spray devices, droplet sources, or evaporation sources. Gels can be based on aqueous or oily matrices and can be formulated to particular needs in terms of stickyness, moisture retention or aging characteristics.

The bait employed in the composition is a product, which is sufficiently attractive to incite insects such as ants, termites, wasps, flies, mosquitos, crickets etc. or cockroaches to eat it. The attractiveness can be manipulated by using feeding stimulants or sex pheromones. Food stimulants are chosen, for example, but not exclusively, from animal and/or plant proteins (meat-, fish or blood meal, insect parts, egg yolk), from fats and oils of animal and/or plant origin, or mono-, oligo or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

For use in bait compositions, the typical content of active ingredient is from 0.001 weight % to 15 weight %, desirably from 0.001 weight % to 5 weight % of the active compound.

Formulations of compounds of the present invention as aerosols (e.g in spray cans), oil sprays or pump sprays are highly suitable for the non-professional user for controlling pests such as flies, fleas, ticks, mosquitos or cockroaches. Aerosol recipes are preferably composed of the active compound, of solvents such as lower alcohols (e.g. methanol, ethanol, propanol, butanol), of ketones (e.g. acetone, methyl ethyl ketone), paraffin hydrocarbons (e.g. kerosenes) having boiling ranges of approximately 50 to 250° C., dimethylformamide, N-methylpyrrolidone, dimethyl sulfoxide, aromatic hydrocarbons such as toluene, xylene, water, furthermore of auxiliaries such as emulsifiers like sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate of stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used. For use in spray compositions, the content of active ingredient ranges from 0.001 to 80 weight %, preferably from 0.01 to 50 weight % and most preferably from 0.01 to 15 weight %.

The compounds of the present invention and their respective compositions can also be used in mosquito and fumigating coils, smoke cartridges, vaporizer plates or long-term vaporizers and also in moth papers, moth pads or other heat-independent vaporizer systems.

Methods to control infectious diseases transmitted by insects (e.g. malaria, dengue and yellow fever, lymphatic filariasis, and leishmaniasis) with compounds of formula (I) and its respective compositions also comprise treating surfaces of huts and houses, air spraying and impregnation of curtains, tents, clothing items, bed nets, tsetse-fly trap or the like. Insecticidal compositions for application to fibers, fabric, knitgoods, nonwovens, netting material or foils and tarpaulins preferably comprise a mixture including the insecticide, optionally a repellent and at least one binder. Suitable repellents for example are NN-Diethyl-meta-toluamide (DEET), NN-diethylphenylacetamide (DEPA), 1-(3-cyclohexan-1-yl-carbonyl)-2-methylpiperine, (2hydroxymethylcyclohexyl) acetic acid lactone, 2-ethyl-1,3-hexandiol, indalone, Methylneodecanamide (MNDA), a pyrethroid not used for insect control such as {(+/−)-3-allyl-2-methyl4-oxocyclopent-2-(+)-enyl-(+)-trans-chrysantemate (Esbiothrin), a repellent derived from or identical with plant extracts like limonene, eugenol, (+)-Eucamalol (1), (−)-1-epi-eucamalol or crude plant extracts from plants like Eucalyptus maculata, Vitex rotundifolia, Cymbopogan martinii, Cymbopogan citratus (lemon grass), Cymopogan nartdus (citronella). Suitable binders are selected for example from polymers and copolymers of vinyl esters of aliphatic acids (such as such as vinyl acetate and vinyl versatate), acrylic and methacrylic esters of alcohols, such as butyl acrylate, 2-ethylhexylacrylate, and methyl acrylate, mono and di-ethylenically unsaturated hydrocarbons, such as styrene, and aliphatic diens, such as butadiene.

The impregnation of curtains and bednets is done in general by dipping the textile material into emulsions or dispersions of the insecticide or spraying them onto the nets.

The compounds of the present invention and their compositions can be used for protecting wooden materials such as trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being (e.g. when the pests invade into houses and public facilities). The compounds of the present invention are applied not only to the surrounding soil surface or into the under-floor soil in order to protect wooden materials but it can also be applied to lumbered articles such as surfaces of the under-floor concrete, alcove posts, beams, plywoods, furniture, etc., wooden articles such as particle boards, half boards, etc. and vinyl articles such as coated electric wires, vinyl sheets, heat insulating material such as styrene foams, etc.

### Seed Treatment

The present invention still further relates to a seed comprising the compounds of the present invention, particularly in an amount ranging from about 0.0001% to about 1% by weight of the seed before treatment.

The compounds of the present invention are particularly useful for the protection of the seed from soil pests and the resulting plant's roots and shoots against soil pests and foliar insects. The protection of the resulting plant's roots and shoots is preferred. More preferred is the protection of resulting plant's shoots from piercing and sucking insects, wherein the protection from aphids is most preferred.

The present invention therefore comprises a method for the protection of seeds from insects, in particular from soil insects and of the seedling's roots and shoots from insects, in particular from soil and foliar insects, said method comprising contacting the seeds before sowing and/or after pregermination with a compound of the present invention. Particularly preferred is a method, wherein the plant's roots and shoots are protected, more preferably a method, wherein the plants shoots are protected from piercing and sucking insects, most preferably a method, wherein the plants shoots are protected from aphids.

The term seed embraces seeds and plant propagules of all kinds including but not limited to seed pieces, suckers, corms, bulbs, fruit, tubers, grains, cuttings, cut shoots and the like and means in a preferred embodiment true seeds.

The term seed treatment comprises all suitable seed treatment techniques known in the art, such as seed dressing, seed coating, seed dusting, seed soaking and seed pelleting.

The present invention also comprises seeds coated with or containing the active compound. The seeds can be coated with seed coating compositions containing the compounds of the present invention. For example, seed coating compositions reported in EP3165092, EP3158864, WO2016198644, WO2016039623, WO2015192923, CA2940002, US2006150489, US2004237395, WO2011028115, EP2229808, WO2007067042, EP1795071, EP1273219, WO200178507, EP1247436, NL1012918 and CA2083415.

The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

Suitable seed is seed of cereals, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

In addition, the compounds of the present invention may be used for treating seed from plants, which tolerate the action of herbicides or fungicides or insecticides owing to breeding, including genetic engineering methods.

For example, the compound of the present invention can be employed in the treatment of seeds from plants, which are resistant to herbicides from the group consisting of the sulfonylureas, imidazolinones, glufosinate-ammonium or glyphosate-isopropylammonium and analogous active substances (see for example, EP242236, EP242246) (WO92/00377) (EP257993, U.S. Pat. No. 5,013,659) or in transgenic crop plants, for example cotton, with the capability of producing Bacillus thuringiensis toxins (Bt toxins) which make the plants resistant to certain pests (EP142924, EP193259),

Furthermore, the compound of the present invention can be used for the treatment of seed from plants, which have modified characteristics in comparison with existing plants, which can be generated for example by traditional breeding methods and/or the generation of mutants, or by recombinant procedures). For example, a number of cases have been described of recombinant modifications of crop plants for the purpose of modifying the starch synthesized in the plants (e.g. WO92/11376, WO92/14827, WO91/19806) or of transgenic crop plants having a modified fatty acid composition (WO91/13972).

The seed treatment application of the compound of the present invention is carried out by spraying or by dusting the seeds before sowing of the plants and before emergence of the plants.

Compositions which are especially useful for seed treatment are e.g.:
A. Soluble concentrates (SL, LS)
B. Emulsions (EW, EO, ES)
C. Suspensions (SC, OD, FS)
D. Water-dispersible granules and water-soluble granules (WG, SG)
E. Water-dispersible powders and water-soluble powders (WP, SP, WS)
F. Gel-Formulations (GF)
G. Dustable powders (DP, DS)

Conventional seed treatment formulations include for example flowable concentrates FS, solutions LS, powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter

In a one embodiment a FS formulation is used for seed treatment. Typically, a FS formulation may comprise 1-800 g/l of active ingredient, 1-200 g/l surfactant, 0 to 200 g/l antifreezing agent, 0 to 400 g/l of binder, 0 to 200 g/l of a pigment and up to 1 liter of a solvent, preferably water.

Especially FS formulations of compounds of the present invention for seed treatment usually comprise from 0.1 to 80% by weight (1 to 800 g/l) of the active ingredient, from 0.1 to 20% by weight (1 to 200 g/l) of at least one surfactant, e.g. 0.05 to 5% by weight of a wetter and from 0.5 to 15% by weight of a dispersing agent, up to 20% by weight, e.g. from 5 to 20% of an anti-freeze agent, from 0 to 15% by weight, e.g. 1 to 15% by weight of a pigment and/or a dye, from 0 to 40% by weight, e.g. 1 to 40% by weight of a binder (sticker/adhesion agent), optionally up to 5% by weight, e.g. from 0.1 to 5% by weight of a thickener, optionally from 0.1 to 2% of an anti-foam agent, and optionally a preservative such as a biocide, antioxidant or the like, e.g. in an amount from 0.01 to 1% by weight and a filler/vehicle up to 100% by weight.

Seed treatment formulations may additionally comprise binders and optionally colorants.

Binders can be added to improve the adhesion of the active materials on the seeds after treatment. Suitable binders are homo and copolymers from alkylene oxides like ethylene oxide or propylene oxide, polyvinylacetate, polyvinylalcohols, polyvinylpyrrolidones, and copolymers thereof, ethylene-vinyl acetate copolymers, acrylic homo and copolymers, polyethyleneamines, polyethyleneamides and polyethylenepyrimidines, polysaccharides like celluloses, tylose and starch, polyolefin homo and copolymers like olefin/maleic anhydride copolymers, polyurethanes, polyesters, polystyrene homo and copolymers.

Optionally, also colorants can be included in the formulation. Suitable colorants or dyes for seed treatment formulations are Rhodamin B, C.I. Pigment Red 1 12, C.I. Solvent Red 1, pigment blue 15:4, pigment blue 15:3, pigment blue 15:2, pigment blue 15:1, pigment blue 80, pigment yellow 1, pigment yellow 13, pigment red 1 12, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

An example of a gelling agent is carrageen (Satiagel®)

In the treatment of seed, the application rates of the compounds of the present invention are generally from 0.1 g to 10 kg per 100 kg of seed, preferably from 1 g to 5 kg per 100 kg of seed, more preferably from 1 g to 1 kg per 100 kg of seed and in particular from 1 g to 200 g per 100 kg of seed. The present invention therefore also provides seed comprising a compound of the formula (I), or an agriculturally useful salt of formula (I), as defined herein. The amount of the compound of formula (I) or the agriculturally useful salt thereof will in general vary from 0.1 g to 10 kg per 100 kg of seed, preferably from 1 g to 5 kg per 100 kg of seed, in particular from 1 g to 1 kg per 100 kg of seed. For specific crops such as lettuce the rate can be higher.

### Animal Health

The present invention also provides an agricultural and/or veterinary composition comprising at least one compound of the present invention.

The present invention still further relates to a use of the compound, N-oxide or veterinarily acceptable salt thereof or the composition of the present invention, for the preparation of a medicament for treating or protecting animals against the infestation or infection by insect and mite pests or parasites.

The compounds of formula (I), their N-oxides and/or veterinarily acceptable salts thereof are also particularly suitable for being used for combating parasites in and on animals.

One object of the present invention is therefore to provide new methods to control parasites in and on animals. Another object of the present invention is to provide safer pesticides for animals. Another object of the present invention is to provide pesticides for animals that may be used in lower doses than existing pesticides. Another object of the present invention is to provide pesticides for animals, which provide a long-lasting control of parasites.

The present invention also relates to compositions containing a parasiticidally effective amount of at least one compound of formula (I), N-oxide or veterinarily acceptable salt thereof and an acceptable carrier, for combating parasites in and on animals.

The present invention also provides a method for treating, controlling, preventing and protecting animals against infestation and infection by parasites, which comprises orally, topically, or parenteral administering or applying to the animals a parasiticidally effective amount of a compound of the present invention or a composition comprising it.

The present invention also provides a process for the preparation of a composition for treating, controlling, preventing or protecting animals against infestation or infection by parasites which comprises a parasiticidally effective amount of a compound of the present invention or a composition comprising it.

Activity of compounds against agricultural pests does not suggest their suitability for control of endo and ectoparasites in and on animals which requires, for example, low, non-emetic dosages in the case of oral application, metabolic compatibility with the animal, low toxicity and a safe handling.

Surprisingly it has now been found that compounds of the present invention are suitable for combating endo- and ectoparasites in and on animals.

Compounds of the present invention and compositions comprising them are preferably used for controlling and preventing infestations and infections of animals including warm-blooded animals (including humans) and fish. They are for example suitable for controlling and preventing infestations and infections in mammals such as cattle, sheep, swine, camels, deer, horses, pigs, poultry, rabbits, goats, dogs and cats, water buffalo, donkeys, fallow deer and reindeer, and also in fur-bearing animals such as mink, chinchilla and raccoon, birds such as hens, geese, turkeys and ducks and fish such as fresh and salt-water fish like trout, carp and eels.

Compounds of the present invention and compositions comprising them are preferably used for controlling and preventing infestations and infections in domestic animals, such as dogs or cats.

Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of the present invention and compositions comprising them are suitable for a systemic and/or non-systemic control of ecto- and/or endo-parasites. They can be active against all or some stages of development.

The compounds of the present invention are especially useful for combating ectoparasites.

The compounds of the present invention are especially useful for combating parasites of the following orders and species, respectively: fleas (Siphonaptera), e.g. Ctenocephalides felis, Ctenocephalides cams, Xenopsylla cheopis, Pulex irritans, Tunga penetrans, and Nosopsyllus fasciatus, cockroaches (Blattaria Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis, flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Chrysomya bezziana, Chrysomya hominivorax, Chrysomya macellaria, Chrysops discalis, Chrysops silacea, Chrysops atlanticus, Cochliomyia hominivorax, Cordylobia anthropophaga, Culicoides furens, Culex pi ens, Culex nigripalpus, Culex quinquefasciatus, Culex tarsalis, Culiseta inornata, Culiseta melanura, Dermatobia hominis, Fannia canicularis, Gasterophilus intestinalis, Glossina morsitans, Glossina palpalis, Glossina fuscipes, Glossina tachinoides, Haematobia irritans, Haplodiplosis equestris, Hippelates spp., Hypoderma lineata, Leptoconops torrens, Lucilia caprina, Lucilia cuprina, Lucilia sericata, Lycoria pectoralis, Mansonia spp., Musca domestica, Muscina stabulans, Oestrus ovis, Phlebotomus argentipes, Psorophora columbiae, Psorophora discolor, Prosimulium mixtum, Sarcophaga haemorrhoidalis, Sarcophaga sp., Simulium vittatum, Stomoxys calcitrans, Tabanus bovinus, Tabanus atratus, Tabanus lineola, and Tabanus similis, lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus. ticks and parasitic mites (Parasitiformes): ticks (Ixodida), e.g. Ixodes scapularis, Ixodes holocyclus, Ixodes pacificus, Rhiphicephalus sanguineus, Dermacentor andersoni, Dermacentor variabilis, Amblyomma americanum, Ambryomma maculatum, Ornithodorus hermsi, Ornithodorus turicata and parasitic mites (Mesostigmata), e.g. Ornithonyssus bacoti and Dermanyssus gallinae, Actinedida (Prostigmata) und Acaridida (Astigmata) e.g. Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., and Laminosioptes spp, Bugs (Heteropterida): Cimex lectularius, Cimex hemipterus, Reduvius senilis, Triatoma spp., Rhodnius ssp., Panstrongylus ssp. and Arilus critatus, Anoplurida, e.g. Haematopinus spp., Linognathus spp., Pediculus spp., Phtirus spp., and Solenopotes spp, Mallophagida (suborders Arnblycerina and Ischnocerina), e.g. Trimenopon spp., Menopon spp., Trinoton spp., Bovicola spp., Werneckiella spp., Lepikentron spp., Trichodectes spp., and Felicola spp.

### Roundworms Nematoda:

Wipeworms and Trichinosis (Trichosyringida), e.g. Trichinellidae (Trichinella spp.), (Trichuridae,) Trichuris spp., Capillaria spp, Rhabditida, e.g. Rhabditis spp, Strongyloides spp., Helicephalobus spp, Strongylida, e.g. Strongylus spp., Ancylostoma spp., Necator americanus, Bunostomum spp. (Hookworm), Trichostrongylus spp., Haemonchus contortus., Ostertagia spp., Cooperia spp., Nematodirus spp., Dictyocaulus spp., Cyathostoma spp., Oesophagostomum spp., Stephanurus dentatus, Ollulanus spp., Chabertia spp., Stephanurus dentatus, Syngamus trachea, Ancylostoma spp., Uncinaria spp., Globocephalus spp., Necator spp., Metastrongylus spp., Muellerius capillaris, Protostrongylus spp., Angiostrongylus spp., Parelaphostrongylus spp. Aleurostrongylus abstrusus, and Dioctophyma renale, Intestinal roundworms (Ascaridida), e.g. Ascaris lumbricoides, Ascaris suum, Ascaridia galli, Parascaris equorum, Enterobius vermicularis (Threadworm), Toxocara canis, Toxascaris leonine, Skrjabinema spp., and Oxyuris equi, Camallanida, e.g. Dracunculus medinensis (guinea worm) Spirurida, e.g. Thelazia spp. Wuchereria spp., Brugia spp., Onchocerca spp., Dirofilari spp. a, Dipetalonema spp., Setaria spp., Elaeophora spp., Spirocerca lupi, and Habronema spp, Thorny headed worms (Acanthocephala), e.g. Acanthocephalus spp., Macracanthorhynchus hirudinaceus and Oncicola spp, Planarians (Plathelminthes): Flukes (Trematoda), e.g. Faciola spp., Fascioloides magna, Paragonimus spp., Dicrocoelium spp., Fasciolopsis buski, Clonorchis sinensis, Schistosoma spp., Trichobilharzia spp., Alaria a lata, Paragonimus spp., and Nanocyetes spp, Cercomeromorpha, in particular Cestoda (Tapeworms), e.g. Diphyllobothrium spp., Tenia spp., Echinococcus spp., Dipylidium caninum, Multiceps spp., Hymenolepis spp., Mesocestoides spp., Vampirolepis spp., Moniezia spp., Anoplocephala spp., Sirometra spp., Anoplocephala spp., and Hymenolepis spp.

The compounds of formula (I) and the compositions containing them are particularly useful for the control of pests from the orders Diptera, Siphonaptera and Ixodida.

Moreover, the use of the compounds of formula (I) and the compositions containing them for combating mosquitoes is one embodiment of the present invention.

The use of the compounds of the present invention and the compositions containing them for combating flies is another embodiment of the present invention.

Furthermore, the use of the compounds of the present invention and the compositions containing them for combating fleas is still another embodiment of the present invention.

The use of the compounds of the present invention and the compositions containing them for combating ticks is still another embodiment of the present invention.

The compounds of the present invention also are especially useful for combating endoparasites (roundworms nematoda, thorny headed worms and planarians).

The administration can be carried out both prophylactically and therapeutically.

The administration of the compounds of the present invention is carried out directly or in the form of suitable preparations, orally, topically/dermally or parenterally.

For an oral administration to warm-blooded animals, the compounds of the present invention may be formulated as animal feeds, animal feed premixes, animal feed concentrates, pills, solutions, pastes, suspensions, drenches, gels, tablets, boluses and capsules. In addition, the compounds of the present invention may be administered to the animals in their drinking water. For oral administration, the dosage form chosen should provide the animal with 0.01 mg/kg to 100 mg/kg of animal body weight per day of the compound of the present invention, preferably with 0.5 mg/kg to 100 mg/kg of animal body weight per day.

Alternatively, the compounds of the present invention may be administered to animals parenterally, for example, by intraruminal, intramuscular, intravenous or subcutaneous injection. The compounds of the present invention may be dispersed or dissolved in a physiologically acceptable carrier for subcutaneous injection. Alternatively, the compounds of the present invention may be formulated into an implant for subcutaneous administration. In addition, the compound of the present invention may be transdermally administered to animals. For parenteral administration, the dosage form chosen should provide the animal with 0.01 mg/kg to 100 mg/kg of animal body weight per day of the compound of the present invention.

The compounds of the present invention may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the compound of the present invention. In addition, the compounds of the present invention may be formulated as ear tags for animals, particularly quadrupeds such as cattle and sheep.

Suitable preparations are: solutions such as oral solutions, concentrates for oral administration after dilution, solutions for use on the skin or in body cavities, pouring-on formulations, gels; emulsions and suspensions for oral or dermal administration; semi-solid preparations; formulations in which the active compound is processed in an ointment base or in an oil-in water or water-in-oil emulsion base; solid preparations such as powders, premixes or concentrates, granules, pellets, tablets, boluses, capsules; aerosols and inhalants, and active compound-containing shaped articles.

Compositions suitable for an injection are prepared by dissolving the active ingredient in a suitable solvent and optionally adding further ingredients such as acids, bases, buffer salts, preservatives, and solubilizers.

The solutions are filtered and filled sterile.

Suitable solvents are physiologically tolerable solvents such as water, alkanols such as ethanol, butanol, benzyl alcohol, glycerol, propylene glycol, polyethylene glycols, N-methylpyrrolidone, 2-pyrrolidone, and mixtures thereof.

The active compounds can optionally be dissolved in physiologically tolerable vegetable or synthetic oils which are suitable for injection.

Suitable solubilizers are the solvents which promote the dissolution of the active compound in the main solvent or prevent its precipitation. Examples are polyvinylpyrrolidone, polyvinyl alcohol, polyoxyethylated castor oil, and polyoxyethylated sorbitan ester.

Suitable preservatives are benzyl alcohol, trichlorobutanol, p-hydroxybenzoic acid esters, and n-butanol.

Oral solutions are administered directly. Concentrates are administered orally after prior dilution to the use concentration. Oral solutions and concentrates are prepared according to the state of the art and as described above for injection solutions, sterile procedures not being necessary.

Solutions for use on the skin are trickled on, spread on, rubbed in, sprinkled or sprayed on.

Solutions for use on the skin are prepared according to the state of the art and according to what is described above for injection solutions, sterile procedures not being necessary.

Further suitable solvents are polypropylene glycol, phenyl ethanol, phenoxy ethanol, ester such as ethyl or butyl acetate, benzyl benzoate, ethers such as alkyleneglycol alkylether, e.g. dipropylenglycol monomethylether, ketones such as acetone, methylethylketone, aromatic hydrocarbons, vegetable and synthetic oils, dimethylformamide, dimethylacetamide, transcutol, solketal, propylencarbonate, and mixtures thereof.

It may be advantageous to add thickeners during preparation. Suitable thickeners are inorganic thickeners such as bentonites, colloidal silicic acid, aluminium monostearate, organic thickeners such as cellulose derivatives, polyvinyl alcohols and their copolymers, acrylates and methacrylates.

Gels are applied to or spread on the skin or introduced into body cavities. Gels are prepared by treating solutions which have been prepared as described in the case of the injection solutions with sufficient thickener that a clear material having an ointment-like consistency results. The thickeners employed are the thickeners given above.

Pour-on formulations are poured or sprayed onto limited areas of the skin. The active compound is penetrating the skin and acting systemically. Pour-on formulations are prepared by dissolving, suspending or emulsifying the active compound in suitable skin-compatible solvents or solvent mixtures. If appropriate, other auxiliaries such as colorants, bio absorption-promoting substances, antioxidants, light stabilizers, adhesives are added.

Suitable solvents are: water, alkanols, glycols, polyethylene glycols, polypropylene glycols, glycerol, aromatic alcohols such as benzyl alcohol, phenylethanol, phenoxyethanol, esters such as ethyl acetate, butyl acetate, benzyl benzoate, ethers such as alkylene glycol alkyl ethers such as dipropylene glycol monomethyl ether, diethylene glycol mono-butyl ether, ketones such as acetone, methyl ethyl ketone, cyclic carbonates such as propylene carbonate, ethylene carbonate, aromatic and/or aliphatic hydrocarbons, vegetable or synthetic oils, DMF, dimethylacetamide, n-alkylpyrrolidones such as methylpyrrolidone, n-butylpyrrolidone or noctylpyrrolidone, N-methylpyrrolidone, 2-pyrrolidone, 2,2-dimethyl-4-oxy-methylene-1,3-dioxolane and glycerol formal.

Suitable colorants are all colorants permitted for the use on animals and which can be dissolved or suspended.

Suitable absorption-promoting substances are, for example, DMSO, spreading oils such as isopropyl myristate, dipropylene glycol pelargonate, silicone oils and copolymers thereof with polyethers, fatty acid esters, triglycerides, fatty alcohols.

Suitable antioxidants are sulfites or metabisulfites such as potassium metabisulfite, ascorbic acid, butylhydroxytoluene, butylhydroxyanisole, tocopherol.

Suitable light stabilizers are, for example, novantisolic acid. Suitable adhesives are, for example, cellulose derivatives, starch derivatives, polyacrylates, natural polymers such as alginates, gelatin.

Emulsions can be administered orally, dermally or as injections. Emulsions are either of the water-in-oil type or of the oil-in-water type.

They are prepared by dissolving the active compound either in the hydrophobic or in the hydrophilic phase and homogenizing this with the solvent of the other phase with the aid of suitable emulsifiers and, if appropriate, other auxiliaries such as colorants, absorption-promoting substances, preservatives, antioxidants, light stabilizers, viscosity-enhancing substances.

### Suitable Hydrophobic Phases (Oils) are:

Liquid paraffins, silicone oils, natural vegetable oils such as sesame oil, almond oil, castor oil, synthetic triglycerides such as caprylic/capric biglyceride, triglyceride mixture with vegetable fatty acids of the chain length Cs-Ci2 or other specially selected natural fatty acids, partial glyceride mixtures of saturated or unsaturated fatty acids possibly also containing hydroxyl groups, mono and diglycerides of the Cs-do fatty acids, fatty acid esters such as ethyl stearate, di-n-butyryl adipate, hexyl laurate, dipropylene glycol perlargonate, esters of a branched fatty acid of medium chain length with saturated fatty alcohols of chain length C16-C18, isopropyl myristate, isopropyl palmitate, caprylic/capric acid esters of saturated fatty alcohols of chain length C12-C18, isopropyl stearate, oleyl oleate, decyl oleate, ethyl oleate, ethyl lactate, waxy fatty acid esters such as synthetic duck coccygeal gland fat, dibutyl phthalate, diisopropyl adipate, and ester mixtures related to the latter, fatty alcohols such as isotridecyl alcohol, 2-octyldodecanol, cetylstearyl alcohol, oleyl alcohol, and fatty acids such as oleic acid and mixtures thereof. Suitable hydrophilic phases are: water, alcohols such as propylene glycol, glycerol, sorbitol and mixtures thereof.

Suitable emulsifiers are: non-ionic surfactants, e.g. polyethoxylated castor oil, polyethoxylated sorbitan monooleate, sorbitan monostearate, glycerol monostearate, polyoxyethyl stearate, alkylphenol polyglycol ether; ampholytic surfactants such as di-sodium N-lauryl-p-iminodipropionate or lecithin.

Suitable Anionic surfactants are sodium lauryl sulfate, fatty alcohol ether sulfates, mono/dialkyl polyglycol ether orthophosphoric acid ester monoethanolamine salt; suitable cation-active surfactants are cetyltrimethylammonium chloride.

Suitable further auxiliaries are: substances which enhance the viscosity and stabilize the emulsion, such as carboxymethylcellulose, methylcellulose and other cellulose and starch derivatives, polyacrylates, alginates, gelatin, gum arabic, polyvinylpyrrolidone, polyvinyl alcohol, copolymers of methyl vinyl ether and maleic anhydride, polyethylene glycols, waxes, colloidal silicic acid or mixtures of the substances mentioned.

Suspensions can be administered orally or topically/dermally. They are prepared by suspending the active compound in a suspending agent, if appropriate with addition of other auxiliaries such as wetting agents, colorants, bioabsorption-promoting substances, preservatives, antioxidants, light stabilizers.

Liquid suspending agents are all homogeneous solvents and solvent mixtures.

Suitable wetting agents (dispersants) are the emulsifiers given above.

Other auxiliaries which may be mentioned are those given above.

Semi-solid preparations can be administered orally or topically/dermally. They differ from the suspensions and emulsions described above only by their higher viscosity.

For the production of solid preparations, the active compound is mixed with suitable excipients, if appropriate with the addition of auxiliaries, and brought into the desired form.

Suitable excipients are all physiologically tolerable solid inert substances. Those used are inorganic and organic substances. Inorganic substances are, for example, sodium chloride, carbonates such as calcium carbonate, hydrogencarbonates, aluminium oxides, titanium oxide, silicic acids, argillaceous earths, precipitated or colloidal silica, or phosphates. Organic substances are, for example, sugar, cellulose, foodstuff and feeds such as milk powder, animal meal, grain meals and shreds, starches.

Suitable auxiliaries are preservatives, antioxidants, and/or colorants which have been mentioned above.

Other suitable auxiliaries are lubricants and glidants such as magnesium stearate, stearic acid, talc, bentonites, disintegration-promoting substances such as starch or crosslinked polyvinylpyrrolidone, binders such as starch, gelatin or linear polyvinylpyrrolidone, and dry binders such as microcrystalline cellulose.

In general, “parasiticidally effective amount” means the amount of active ingredient needed to achieve an observable effect on the growth, including death, retardation, prevention, removal and destruction, or otherwise diminishing the occurrence and activity of the target organisms. The parasiticidally effective amount can vary for the various compounds/compositions used in the present invention. A parasiticidally effective amount of the compositions will also vary according to the prevailing conditions such as desired parasiticidal effect and duration, target species, mode of application, and the like. The compositions which can be used in the present invention generally comprise from about 0.001 to 95% of the compound of the present invention.

Generally, it is favorable to apply the compounds of the present invention in total amounts of 0.5 mg/kg to 100 mg/kg per day, preferably 1 mg/kg to 50 mg/kg per day. Ready-to-use preparations contain the compounds acting against parasites, preferably ectoparasites, in concentrations of 10 ppm to 80 percent by weight, preferably from 0.1 to 65 percent by weight, more preferably from 1 to 50 percent by weight, most preferably from 5 to 40 percent by weight. Preparations are diluted before use containing the compounds acting against ectoparasites in concentrations of 0.5 to 90 percent by weight, preferably of 1 to 50 percent by weight. Furthermore, the preparations comprise the compounds of the present invention against endoparasites in concentrations of 10 ppm to 2 percent by weight, preferably of 0.05 to 0.9 percent by weight, very particularly preferably of 0.005 to 0.25 percent by weight.

In a one embodiment, the compositions comprising the compounds of the present invention are applied dermally/topically.

In another embodiment, the topical application is conducted in the form of compound-containing shaped articles such as collars, medallions, ear tags, bands for fixing at body parts, and adhesive strips and foils.

Generally, it is favorable to apply solid formulations which release compounds of the present invention in total amounts of 10 mg/kg to 300 mg/kg, preferably 20 mg/kg to 200 mg/kg, most preferably 25 mg/kg to 160 mg/kg body weight of the treated animal in the course of three weeks.

For the preparation of the shaped articles, thermoplastic and flexible plastics as well as elastomers and thermoplastic elastomers are used. Suitable plastics and elastomers are polyvinyl resins, polyurethane, polyacrylate, epoxy resins, cellulose, cellulose derivatives, polyamides and polyester which are sufficiently compatible with the compounds of the present invention. A detailed list of plastics and elastomers as well as preparation procedures for the shaped articles is given e.g. in WO 2003/086075.

### Digital Technologies

The compounds of the invention can be used in combination with models e.g. embedded in computer programs for site specific crop management, satellite farming, precision farming or precision agriculture. Such models support a site-specific management of agricultural sites with data from various sources such as soils, weather, crops (e.g. type, growth stage, plant health), weeds (e.g. type, growth stage), diseases, pests, nutrients, water, moisture, biomass, satellite data, yield etc. with the purpose to optimize profitability, sustainability and protection of the environment. Particularly, such models can help to optimize agronomical decisions, control the precision of pesticide applications and record the work performed.

As an example, the compounds of the invention can be applied to a crop plant according to an appropriate dose regime if a model models the development of a pest and calculates that a threshold has been reached for which it is recommendable to apply the compound of the invention to the crop plant. Commercially available systems which include agronomic models are e.g. FieldScripts™ from The Climate Corporation, Xarvio™ from BASF, AGLogic™ from John Deere, etc.

The compounds of the invention can also be used in combination with smart spraying equipment such as e.g. spot spraying or precision spraying equipment attached to or housed within a farm vehicle such as a tractor, robot, helicopter, airplane, unmanned aerial vehicle (UAV) such as a drone, etc. Such an equipment usually includes input sensors (such as e.g. a camera) and a processing unit configured to analyze the input data and provide a decision, based on the analysis of the input data, to apply the compound of the invention to the crop plants (respectively the weeds) in a specific and precise manner. The use of such smart spraying equipment usually also requires position systems (e.g. GPS receivers) to localize recorded data and to guide or to control farm vehicles; geographic information systems (GIS) to represent the information on intelligible maps, and appropriate farm vehicles to perform the required farm action such as the spraying.

In an example, pests can be detected from imagery acquired by a camera. In an example the pests can be identified and/or classified based on that imagery. Such identification and/or classification can make use of image processing algorithms. Such image processing algorithms can utilize machine learning algorithms, such as trained neutral networks, decision trees and artificial intelligence algorithms. In this manner, the compounds described herein can be applied only where needed.

### Positive Crop Response:

The compounds of the present invention not only control insect and mite pests effectively but also show positive crop response such as plant growth enhancement effects like enhanced root growth, enhanced tolerant to drought, high salt, high temperature, chill, frost or light radiation, improved flowering, enhanced nutrient utilization (such as improved nitrogen assimilation), enhanced quality plant products, more number of productive tillers, enhanced resistance to insect pests and the like, which results in higher yields.

### General Synthesis Scheme:

The invention will now be illustrated in further details by the following examples, without imposing any limitation thereto.

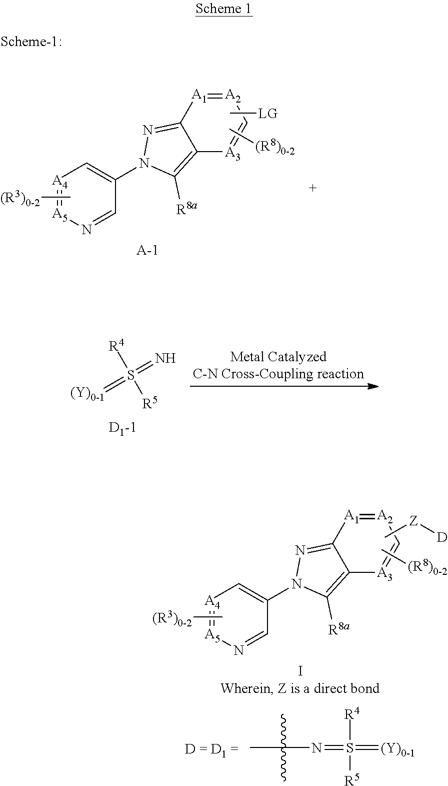

In one of the preferred embodiments, the compounds of formula (I), wherein D represents D₁ and Z is a direct bond, can be prepared by metal catalyzed cross-coupling reactions between the compounds of formula A-1, wherein LG represents a leaving group, and compounds of the formula D₁-1 following the procedure as described in the literature US 20190297887 and in further literature cited therein. The compounds of the formula D₁-1 can either be obtained commercially or synthesized by the method as described in the Chem. Eur. J. 2021, 27, 17293-17321, European Journal of Medicinal Chemistry 2017, 126, 225-245, Chem. Commun., 2017, 53, 348-351, J Am. Chem. Soc. 2020, 142(36), 15445-15453 and in further literature cited therein. Unless stated otherwise the definition of each variable is as defined above for the compounds of formula (I).

The C—N coupling reactions are usually carried out in the presence of solvents and the solvents which can be used for this reaction are not particularly limited, as long as it does not adversely affect the reaction. For example, ethers such as dioxane, tetrahydrofuran, ethylene glycol, dimethyl ether and diethylene glycol dimethyl ether; aromatic hydrocarbons such as benzene, toluene and xylene; N-amides such as N,N-dimethylformamide, N-dimethylacetamide and 1-methyl-2-pyrrolidone; alcohols such as methanol, ethanol, propanol, butanol, 2-propanol and 2-methyl-2-propanol; nitriles such as acetonitrile; or water or a mixture thereof may serve this purpose. Preferable solvents include aromatic hydrocarbons such as toluene and xylene; and amides such as N,N-dimethylformamide, N,N-dimethylacetamide and 1-methyl-2-pyrrolidone; out of which NN-dimethylformamide and toluene are most preferable. The reaction is carried out in the presence of a base selected from, but not limited to, metal alkoxides such as sodium methoxide, sodium ethoxide, potassium tert-butoxide, and sodium tert-butoxide; inorganic bases such as sodium hydroxide, potassium hydroxide, sodium carbonate, potassium carbonate, cesium carbonate, sodium phosphate, potassium phosphate, potassium hydrogenphosphate, and potassium hydride; organic bases such as triethylamine, N,N-diisopropylethylamine and pyridine. Preferred bases include metal alkoxides such as potassium tert-butoxide and sodium tert-butoxide. The palladium catalysts that can be used for this reaction include, but are not limited to, palladium-carbon; inorganic palladium salts such as palladium chloride; organic palladium complexes such as palladium acetate; tetrakis (triphenylphosphine) palladium (0), bis (triphenylphosphine) palladium (II) chloride, 1,1′-bis(diphenylphosphino) phenylpalladium (II) chloride, and tris (dibenzylidene acetone) dipalladium (0); and a polymer-immobilized organic palladium complex such as a polymer-supported bis (acetato) triphenyl phosphine palladium (II) and a polymer-carrying di (acetato) dicyclohexylphosphine palladium (II). Palladium catalysts such as palladium chloride, palladium acetate, tetrakis (triphenylphosphine) palladium (0), bis (triphenylphosphine) palladium (II) chloride, 1′-bis(diphenylphosphino) phenylpalladium (II) chloride and tris (dibenzylidene acetone) dipalladium (0) are more preferable. Examples of ligands that can be used for this reaction include, but are not limited to, tris(o-tolyl)phosphine, tricyclohexylphosphine, tri-tert-butylphosphonium tetrafluoroborate, (oxydi-2,1-phenylene)bis[dicyclohexyl]phosphine, 4,5-bis(diphenylphosphino)-9,9-dimethylxanthene, 2-dicyclohexylphosphino-2′-(N,N-dimethylamino)biphenyl, 2-dicyclohexylphosphino-2′,4′,6′-triisopropylbiphenyl, 2-dicyclohexylphosphino-2′,6′-dimethoxybiphenyl, 2-dicyclohexylphosphino-2′,6′-diisopropoxybiphenyl, 2-di-tert-butylphosphino-2′,4′,6′-triisopropylbiphenyl, 2-di-tert-butylphosphino-3,4,5,6-tetramethyl-2′,4′,6′-triisopropyl-1,1′-biphenyl, (2-biphenylyl) di-tert-butylphosphine; 2-(di-tert-butylphosphino)biphenyl, 2-di-tert-butylphosphino-3,4,5,6-tetramethyl-2′,4′,6′-triisopropyl-1,1′-biphenyl, 1,1′-ferrocenediyl-bis(diphenylphosphine), 2-di-tert-butylphosphino-2′-methylbiphenyl, 2-methyl-2′-dicyclohexylphosphinobiphenyl and [1,1′-biphenyl]-3-sulfonic acid, 2′-(dicyclohexylphosphino)-2,6-dimethoxy-sodium salt. The preferred ligands include 2-(di-tert-butylphosphino)biphenyl, 2-di-tert-butylphosphino-3,4,5,6-tetramethyl-2′,4′,6′-triisopropyl-1,1′-biphenyl (Oxydi-2,1-phenylene)bis[dicyclohexyl]phosphine, 1,1′-ferrocenediyl-bis(diphenylphosphine and 4,5-bis(diphenylphosphino)-9,9-dimethylxanthene. The reaction can be performed at a temperature in the range of 0-200° C., preferably at a temperature in the range of 50-150° C. for a duration in the range of 30 minutes to 24 hours.

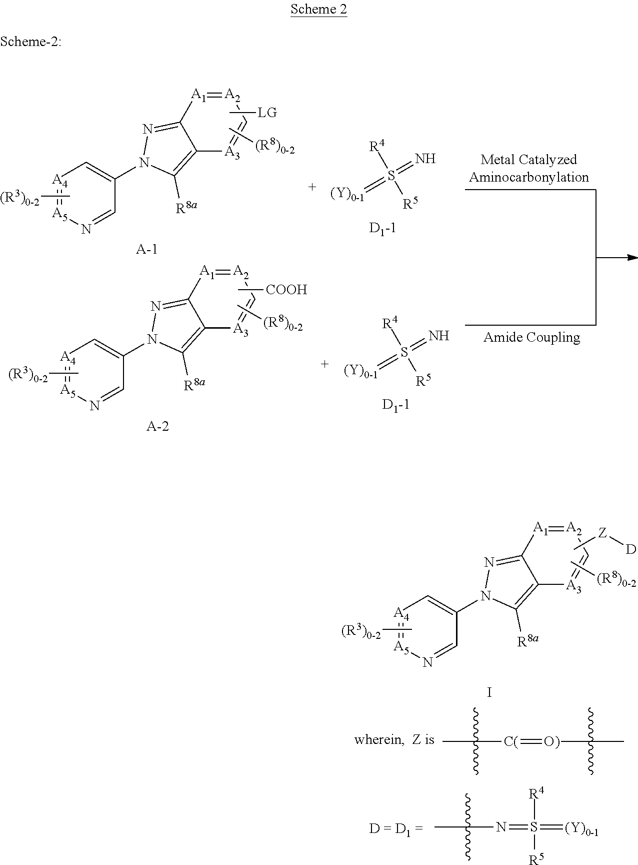

In another preferred embodiment, the compounds of formula (I), wherein D represents D₁ and Z is —C═O, can be prepared by either metal catalyzed aminocarbonylation reactions, or by amide coupling reactions between the compounds of formula A-1 (A-2 for amide coupling reactions), wherein LG represents a leaving group, and compounds of the formula D₁-1, following the procedure as described in the literature WO2016144351, Chem. Eur. J. 2021, 27, 17293-17321 and in further literature cited therein. The compounds of the formula D₁-1 can either be obtained commercially or synthesized by the method as described in the Chem. Eur. J. 2021, 27, 17293-17321, European Journal of Medicinal Chemistry 2017, 126, 225-245, Chem. Commun., 2017, 53, 348-351, J Am. Chem. Soc. 2020, 142, 36, 15445-15453 and in further literature cited therein. Unless stated otherwise, the definition of each variable is as defined above for the compounds of formula (I).

The metal catalyzed aminocarbonylation reaction, as depicted in the scheme 2, is carried out in the presence of a solvent, such as, but not limited to, N-amides, with for example: N, N-dimethylformamide, N-dimethylacetamide and 1-methyl-2-pyrrolidone; N,N-dimethylformamide being the preferred solvents. The metal hexacarbonyls that serve as “CO surrogate” in this reaction are selected from, but are not limited to, molybdenum hexacarbonyl and tungsten hexacarbonyl. The reaction is carried out in the presence of a base selected from, but not limited to, metal hydrides, such as sodium hydride, lithium hydride, and potassium hydride; metal carbonates, such as, sodium carbonate, potassium carbonate, cesium carbonate; organic amine bases, such as triethylamine, N,N-diisopropylethylamine, 1,8-diazabicyclo[5.4.0]undec-7-ene; and preferably in the presence of organic amines such as N,N-diisopropylethylamine, and 1,8-diazabicyclo[5.4.0]undec-7-ene. The catalysts that can be used for this reaction include for example, but are not limited to, organic palladium complexes such as a polymer-supported bis (acetato) triphenyl phosphine palladium (II), trans-di-μ-acetatobis[2-[bis(2-methylphenyl)phosphino]benzyl]dipalladium, and a polymer-carrying di (acetato) dicyclohexylphosphine palladium (II). One examples of ligands used for this reaction tri-tert-butylphosphonium tetrafluoroborate. This reaction is typically carried out within a temperature range of 0-200° C., preferably 50-180° C. for a duration in the range of 30 minutes to 24 hours. The reaction can optionally also be performed under microwave irradiation conditions.

The amide coupling reactions, as depicted in scheme 2, are usually carried out in the presence of a solvent, such as, but not limited to, N-amides, for example N,N-dimethylformamide, N-dimethylacetamide and 1-methyl-2-pyrrolidone; with N,N-dimethylformamide being the preferred solvent. Furthermore, these reactions are carried out in the presence of a base selected from, but not limited to, organic amine bases, such as triethylamine, N,N-diisopropylethylamine, 1,8-diazabicyclo[5.4.0]undec-7-ene, with triethylamine and N,N-diisopropylethylamine being the preferred bases. Coupling reagents that can be used for this reaction are, but not limited to, N,N′-dicyclohexylcarbodiimide, 1-[3-(dimethylamino)propyl]-3-ethylcarbodiimide methiodide, 1-hydroxybenzotriazole hydrate, 1-[bis(dimethylamino)methylene]-1H-1,2,3-triazolo[4,5-b]pyridinium 3-oxid hexafluorophosphate, and N-[(dimethylamino)-1H-1,2,3-triazolo-[4,5-b]pyridin-1-ylmethylene]-N-methylmethanaminium hexafluorophosphate N-oxide. This reaction can be performed at a temperature in the range of 0-200° C., preferably at 0-50° C. for a duration in the range of 30 minutes to 24 hours.

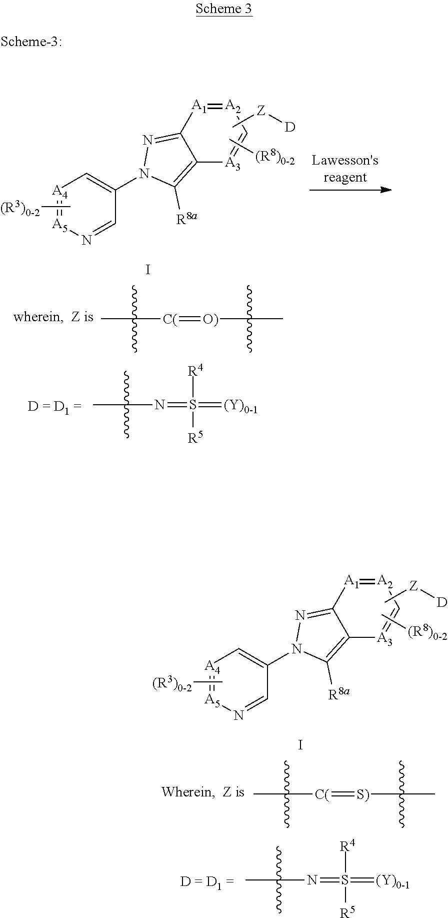

In another preferred embodiment, the compounds of formula (I), wherein D represents D₁ and Z is —C=S, can be prepared starting from the compounds of formula (I), wherein D represents D₁ and Z represents —C═O, in the presence of the Lawesson's reagent under appropriate thioacylation reaction conditions, as described in the literature in Chem. Sci., 2021, 12, 6393-6405 and in further literature cited therein. Unless stated otherwise, the definition of each variable is as defined above for the compounds of formula (I). The thioacylation reactions, as depicted in the scheme 3, is carried out in the presence of a solvent, such as, but not limited to, aromatic hydrocarbons such as benzene, toluene and xylene; preferably in toluene. This reaction can be performed at a temperature in the range of 0-200° C., preferably at 50-150° C. for a duration in the range of 30 minutes to 24 hours.

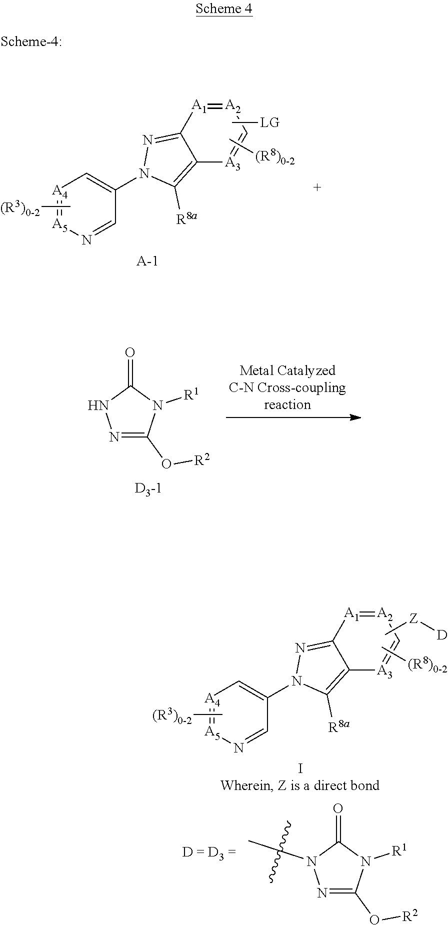

In another preferred embodiment, the compounds of formula (I), wherein D represents D₃ and Z is a direct bond, can be prepared by metal catalyzed cross-coupling reactions between the compounds of formula A-1, wherein LG represents a leaving group, and alkoxytrizolones of the formula D₃-1 following the procedure as described in the literature in US 20190297887 and in further literature cited therein. The alkoxytrizolones of the formula D₃-1 can either be obtained commercially or synthesized by the method as described in U.S. Ser. No. 005606070A and in further literature cited therein. Unless stated otherwise the definition of each variable is as defined above for the compound of formula (I).

The reaction is carried out in the presence of a solvent, such as, but not limited to, ethers such as dioxane, tetrahydrofuran, 2-methyl-tetrahydrofuran, ethylene glycol dimethyl ether and diethylene glycol dimethyl ether; aromatic hydrocarbons such as benzene, toluene and xylene; N, N-dimethylformamide, N-amides such as N-dimethylacetamide and 1-methyl-2-pyrrolidone; alcohols such as methanol, ethanol, propanol, butanol, 2-propanol, 2-methyl-2-propanol, and 2-methyl-2-butanol; nitriles such as acetonitrile; and water or a mixture thereof. Preferable solvents include amides such as N,N-dimethylformamide, N,N-dimethylacetamide and 1-methyl-2-pyrrolidone, and alcohols such as methanol, ethanol, propanol, butanol, 2-propanol, 2-methyl-2-butanol, and 2-methyl-2-propanol; N,N-dimethylformamide, and 2-methyl-2-butanol.

The bases that can be used in this reaction include, but are not limited to, metal alkoxides such as sodium methoxide, sodium ethoxide, potassium tert-butoxide, and sodium tert-butoxide; inorganic bases such as sodium hydroxide, potassium hydroxide, sodium carbonate, potassium carbonate, cesium carbonate, sodium phosphate, potassium phosphate, potassium hydrogenphosphate, and potassium hydride, and organic bases such as triethylamine, N,N-diisopropylethylamine and pyridine. Preferred bases include phosphates such as sodium phosphate, potassium phosphate, potassium hydrogenphosphate and carbonates such as sodium carbonate, potassium carbonate, and cesium carbonate; wherein carbonates such as sodium carbonate, potassium carbonate, and cesium carbonate are more preferable bases. The palladium catalysts that can be used for this reaction include, but are not limited to, palladium-carbon; inorganic palladium salts such as palladium chloride; organic palladium complexes such as palladium acetate; tetrakis (triphenylphosphine) palladium (0), bis(triphenylphosphine) palladium (II) chloride, 1,1′-bis(diphenylphosphino) phenylpalladium (II) chloride, and tris (dibenzylidene acetone) dipalladium (0); and polymer-immobilized organic palladium complexes such as a polymer-supported bis (acetato) triphenyl phosphine palladium (II) and a polymer-carrying di (acetato) dicyclohexylphosphine palladium (II). The palladium catalysts such as palladium chloride, palladium acetate, tetrakis (triphenylphosphine) palladium (0), bis(triphenylphosphine) palladium (II) chloride, 1′-bis(diphenylphosphino) phenylpalladium (II) chloride and tris (dibenzylidene acetone) dipalladium (0) are more preferable. Examples of ligands that can be used for this reaction include, but are not limited to, tris(o-tolyl)phosphine, tricyclohexylphosphine, tri-tert-butylphosphonium tetrafluoroborate, (oxydi-2,1-phenylene)bis[dicyclohexyl]phosphine, 4,5-bis(diphenylphosphino)-9,9-dimethylxanthene, 2-dicyclohexylphosphino-2′-(N,N-dimethylamino)biphenyl, 2-dicyclohexylphosphino-2′,4′,6′-triisopropylbiphenyl, 2-dicyclohexylphosphino-2′,6′-dimethoxybiphenyl, 5-(di-tert-butylphosphino)-1′,3′,5′-triphenyl-1′H-[1,4′]bipyrazole, 5-(dicyclohexylphosphino)-1′,3′,5′-triphenyl-1′H-[1,4′]bipyrazole, 5-[di(1-adamantyl)phosphino]-1′,3′,5′-triphenyl-1′H-[1,4′]bipyrazole, 2-dicyclohexylphosphino-2′,6′-diisopropoxybiphenyl, 2-di-tert-butylphosphino-2′,4′,6′-triisopropylbiphenyl, 2-di-tert-butylphosphino-3,4,5,6-tetramethyl-2′,4′,6′-triisopropyl-1,1′-biphenyl, (2-biphenylyl) di-tert-butylphosphine, 2-(di-tert-butylphosphino)biphenyl, 2-di-tert-butylphosphino-3,4,5,6-tetramethyl-2′,4′,6′-triisopropyl-1,1′-biphenyl, 1,1′-ferrocenediyl-bis(diphenylphosphine), 2-di-tert-butylphosphino-2′-methylbiphenyl, 2-methyl-2′-dicyclohexylphosphinobiphenyl, [1,1′-biphenyl]-3-sulfonic acid, 2′-(dicyclohexylphosphino)-2,6-dimethoxy-sodium salt. The preferred ligands includes 2-(di-tert-butylphosphino)biphenyl, 5-(di-tert-butylphosphino)-1′,3′,5′-triphenyl-1′H-[1,4′]bipyrazole, 5-(dicyclohexylphosphino)-1′,3′,5′-triphenyl-1′H-[1,4′]bipyrazole, 5-[di(1-adamantyl)phosphino]-1′,3′,5′-triphenyl-1′H-[1,4′]bipyrazole, 1,1′-ferrocenediyl-bis(diphenylphosphine and 4,5-is(diphenylphosphino)-9,9-dimethylxanthene. The reaction is carried out at a temperature in the range of 0-200° C., preferably 50-150° C. for a duration in the range of 30 min to 24 hours.

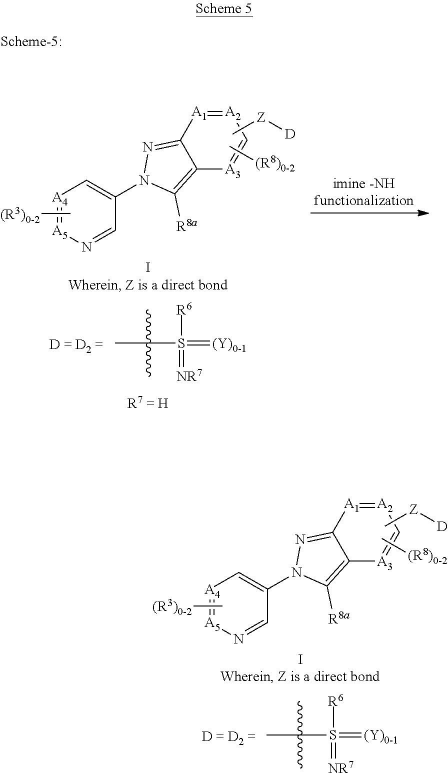

In another preferred embodiment, the compounds of formula (I), wherein D represents D₂, Z is a direct bond, and R⁷ represents hydrogen can be further derivatized at the imine —NH functional group, using reactions such as, but not limited to, N-(halo)alkylation, N-(hetero)arylation, N-acylation, N-amidation, N-halogenation, and N-cyanation to form compounds of the formula (I), wherein D represents D₂, Z represents a direct bond, and R⁷ represents its usual meaning excluding hydrogen, following suitable methods as described in the literature, for example, European Journal of Medicinal Chemistry 2017, 126, 225-245, Org. Lett. 2015, 17, 12, 3166-3169, Tetrahedron Letters 56 (2015) 7056-7058, Eur. J. Org. Chem. 2012, 20, 3737-3741, and WO2019150219 and further literature cited therein. Unless stated otherwise the definition of each variable is defined as above.

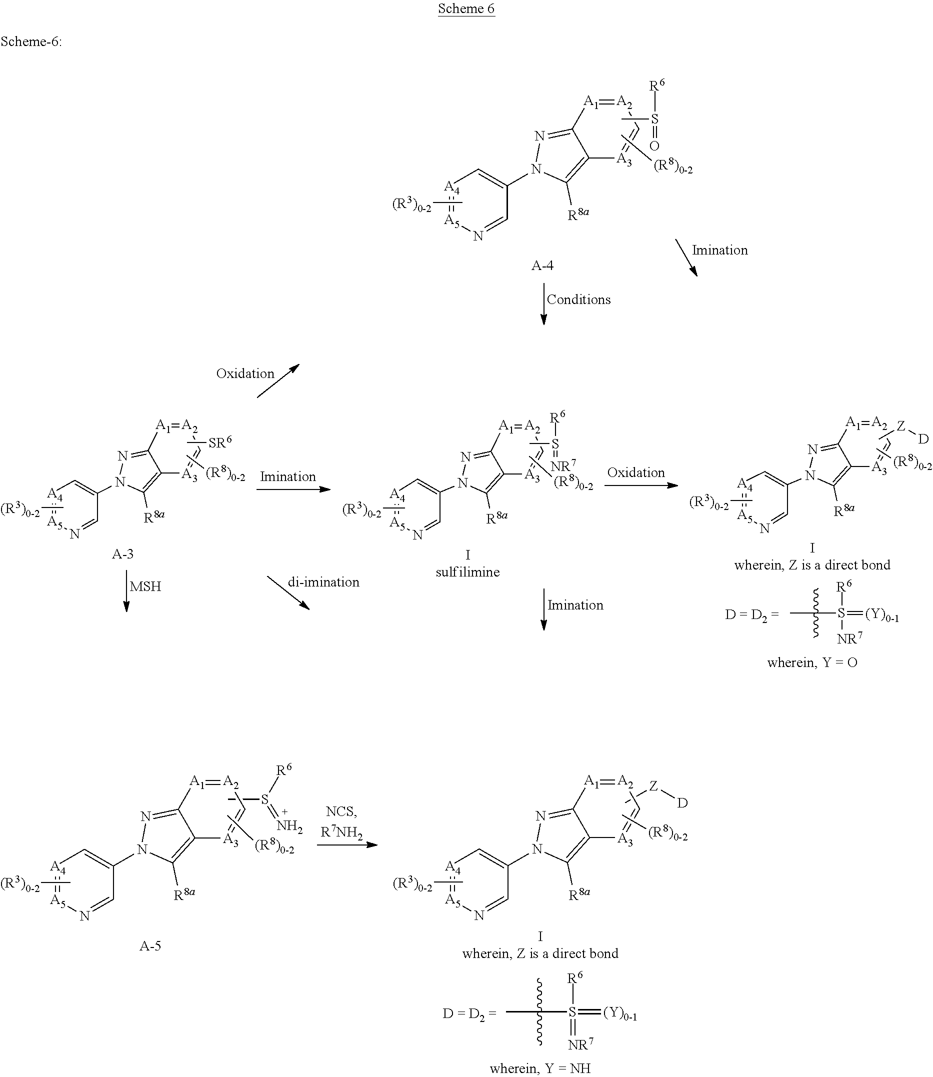

In another preferred embodiment, a variety of sulfur containing representatives of the compounds of formula (I), such as, but not limited to, sulfoximines, sulfilimines, and sulfondiimides, represented by the formula (I), wherein D represents D₂, Z is a direct bond, and R′ is as defined herein above, can be prepared starting from the compounds of formula A-3, following the procedure as described in the literature, for example in the European Journal of Medicinal Chemistry 2017, 126, 225-245 and Chem. Commun., 2017, 53, 348-351 and in further literature cited therein. Unless stated otherwise, the definition of each variable is as defined above for the compounds of formula (I). The sulfoximines of the formula (I) can be prepared starting from sulfides of the formula A-3 in the presence of ammonium carbamate and diacetoxyiodobenzene (see, for example, Angew. Chem. Int. Ed. 2016, 55, 7203-7207). Alternatively, sulfoximines (I) can also be synthesized via the oxidation of sulfilimines (I) (see, for example, Chem. Rev. 1977, 77, 409-435). On the other hand, sulfilimines of the formula (I) can be obtained from sulfides of formula (A-3) via imination reaction using electrophilic aminating reagents, such as, O-(mesitylsulfonyl)hydroxylamine (MSH) or O-(2,4-dinitrophenyl)hydroxylamine (DPH), following the procedure as described in the literature for example in the Angew. Chem. Int. Ed. 2012, 51, 4440-4443 and in the further literature cited therein. Sulfilimines of the formula (I) can also be prepared from the sulfoxides of formula (A-4) via imination using either a) Burgess-type reagents, as described in the literature for example in the Adv. Synth. Catal. 2013, 355, 3363-3368 and in further literature cited therein, or b) Rhodium-catalyzed conditions in presence of electrophilic aminating reagents, such as, but not limited to, O-(mesitylsulfonyl)hydroxylamine (MSH) or O-(2,4-dinitrophenyl)hydroxylamine (DPH), as described in the literature for example in the Chem. Commun., 2014, 50, 9687-9689 and in further literature cited therein. Sulfondiimides of the formula (I) can be synthesized by using either a) direct imination of sulfides of the formula A-3 as described in the literature, for example in the Chem. Ber. 1984, 117, 2779-2784 and in further literature cited therein, or b) imination of sulfilimines of the formula (I) as described in the literature for example in the Angew. Chem. Int. Ed. 2012, 51, 4440-4443 and in the further literature cited therein, or c) imination of the sulfiliminium salts, as represented by the formula A-5, using N-chlorosuccinimide and amines as described in the literature for example in the Angew. Chem. Int. Ed. 2012, 51, 4440-4443 and in further literature cited therein.

According to a feature of the present invention, the compounds of formula A-3 wherein all substituents are as defined earlier, can be prepared by the method given in scheme 7, scheme 8, scheme 9 or as disclosed in the experimental examples. Representative procedures are shown below; however, this disclosure should not be interpreted as to limit the scope of the invention for synthesizing compounds of formula A-3.

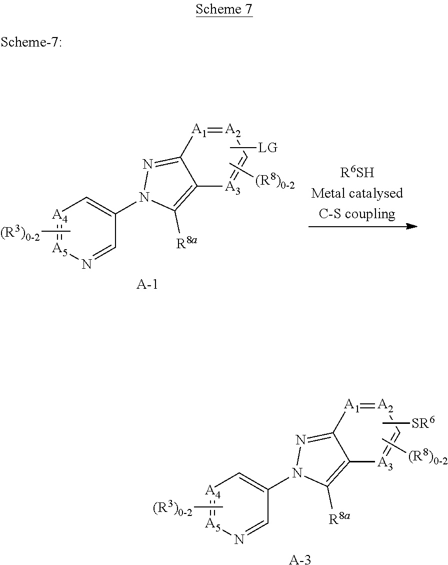

According to the scheme 7, the compound represented by the formula A-3 can be prepared by a base mediated, metal-catalyzed C—S coupling reaction between the compounds of the formula A-1 and thiols of the formula R⁶SH as described in WO2020257145 and in further literature cited therein. The thiols of the formula R⁶SH can be obtained commercially. Unless stated otherwise the definition of each of the variables A₁, A₂, A₃, A₄, A₅, R³, R⁶, R⁸ and R^{8a} is as defined above. LG represents leaving groups, such as halogens or tosylates.

The general protocol for the C—S coupling: the reaction is usually carried out in the presence of a solvent, such as, ethers such as dioxane, tetrahydrofuran, 2-methyl-tetrahydrofuran, methylene glycol dimethyl ether and diethylene glycol dimethyl ether; aromatic hydrocarbons such as benzene, toluene and xylene; N, N-dimethylformamide, N-amides such as N-dimethylacetamide and 1-methyl-2-pyrrolidone; alcohols such as methanol, ethanol, propanol, butanol, 2-propanol, 2-methyl-2-propanol, and 2-methyl-2-butanol; nitriles such as acetonitrile; and water or a mixture thereof. Preferable solvents include amides such as N,N-dimethylformamide, N,N-dimethylacetamide and 1-methyl-2-pyrrolidone, and aromatic hydrocarbons such as benzene, toluene and xylene; with N,N-dimethylformamide and toluene being the most preferable solvents. The reaction is carried out in the presence of a base selected in a non-limiting way from, metal alkoxides such as sodium methoxides, sodium ethoxides, potassium tert-butoxide, and sodium tert-butoxide; inorganic bases, such as, sodium hydroxide, potassium hydroxide, sodium carbonate, potassium carbonate, cesium carbonate, sodium phosphate, potassium phosphate, potassium hydrogenphosphate, and potassium hydride, and organic bases such as triethylamine, N,N-diisopropylethylamine and pyridine. Preferred bases include metal alkoxides such as sodium methoxides, sodium ethoxides, potassium tert-butoxide, sodium tert-butoxide and carbonates such as sodium carbonate, potassium carbonate, and cesium carbonate; wherein carbonates such as cesium carbonate and metal alkoxides such as sodium tert-butoxide and potassium tert-butoxide being most preferable. The palladium catalysts that can be used for this reaction are, for example, but not limited to, palladium-carbon; inorganic palladium salts such as palladium chloride; organic palladium complexes such as palladium acetate; tetrakis (triphenylphosphine) palladium (0), bis (triphenylphosphine) palladium (II) chloride, 1,1′-bis(diphenylphosphino) phenylpalladium (II) chloride, and tris (dibenzylidene acetone) dipalladium (0); and polymer-immobilized organic palladium complexes such as a polymer-supported bis (acetato) triphenyl phosphine palladium (II) and a polymer-carrying di (acetato) dicyclohexylphosphine palladium (II). The palladium catalysts such as palladium chloride, palladium acetate, tetrakis (triphenylphosphine) palladium (0), bis(triphenylphosphine) palladium (II) chloride, 1′-bis(diphenylphosphino) phenylpalladium (II) chloride and tris (dibenzylidene acetone) dipalladium (0) are more preferable. Ligands that can be used for this reaction include, but are not limited to, tris(o-tolyl)phosphine, tricyclohexylphosphine, tri-tert-butylphosphonium tetrafluoroborate, (oxydi-2,1-phenylene)bis[dicyclohexyl]phosphine, 4,5-bis(diphenylphosphino)-9,9-dimethylxanthene, 2-dicyclohexylphosphino-2′-(N,N-dimethylamino)biphenyl, 2-dicyclohexylphosphino-2′,4′,6′-triisopropylbiphenyl, 2-dicyclohexylphosphino-2′,6′-dimethoxybiphenyl, 5-(di-tert-butylphosphino)-1′,3′,5′-triphenyl-1′H-[1,4′]bipyrazole, 5-(dicyclohexylphosphino)-1′,3′,5′-triphenyl-1′H-[1,4′]bipyrazole, 5-[di(1-adamantyl)phosphino]-1′,3′,5′-triphenyl-1′H-[1,4′]bipyrazole, 2-dicyclohexylphosphino-2′,6′-diisopropoxybiphenyl, 2-di-tert-butylphosphino-2′,4′,6′-triisopropylbiphenyl, 2-di-tert-butylphosphino-3,4,5,6-tetramethyl-2′,4′,6′-triisopropyl-1,1′-biphenyl, (2-biphenylyl) di-tert-butylphosphine, 2-(di-tert-butylphosphino)biphenyl, 2-di-tert-butylphosphino-3,4,5,6-tetramethyl-2′,4′,6′-triisopropyl-1,1′-biphenyl, 1,1′-ferrocenediyl-bis(diphenylphosphine), 2-di-tert-butylphosphino-2′-methylbiphenyl, 2-methyl-2′-dicyclohexylphosphinobiphenyl, [1,1′-biphenyl]-3-sulfonic acid, 2′-(dicyclohexylphosphino)-2,6-dimethoxy-sodium salt. The preferred ligands include 2-(di-tert-butylphosphino)biphenyl, 5-(di-tert-butylphosphino)-1′,3′,5′-triphenyl-1′H-[1,4′]bipyrazole, 5-(dicyclohexylphosphino)-1′,3′,5′-triphenyl-1′H-[1,4′]bipyrazole, 5-[di(1-adamantyl)phosphino]-1′,3′,5′-triphenyl-1′H-[1,4′]bipyrazole, 1,1′-ferrocenediyl-bis(diphenylphosphine, and 4,5-Bis(diphenylphosphino)-9,9-dimethylxanthene. The reaction is typically carried out within a temperature range of 0-200° C., preferably 50-150° C. for a duration in the range of 30 minutes to 24 hours.

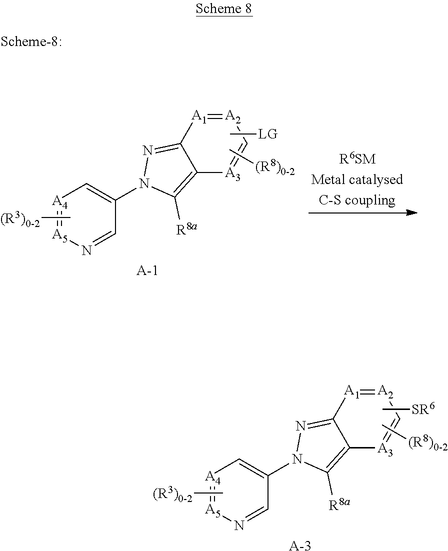

Alternatively, compounds of the formula A-3 can also be prepared, as depicted in the scheme 8, by following the “general protocol for the C—S coupling” as described above. The compounds of the formula A-1 and appropriate metal thiolates of the formula R⁶SM can afford compounds of the formula A-3 as described in the literature in WO2020257145 and in further literature cited therein. Metal thiolates of the formula R⁶SM, where M can be, but is not limited to, sodium or potassium, can either be obtained commercially or can be generated in situ upon treatment of the corresponding thiols with organic and inorganic bases. Unless stated otherwise, the definition of each of the variables A₁, A₂, A₃, A₄, A₅, R³, R⁶, R⁸ and R^{8a} is as defined above. LG represents a leaving group, such as halogens or tosylates.

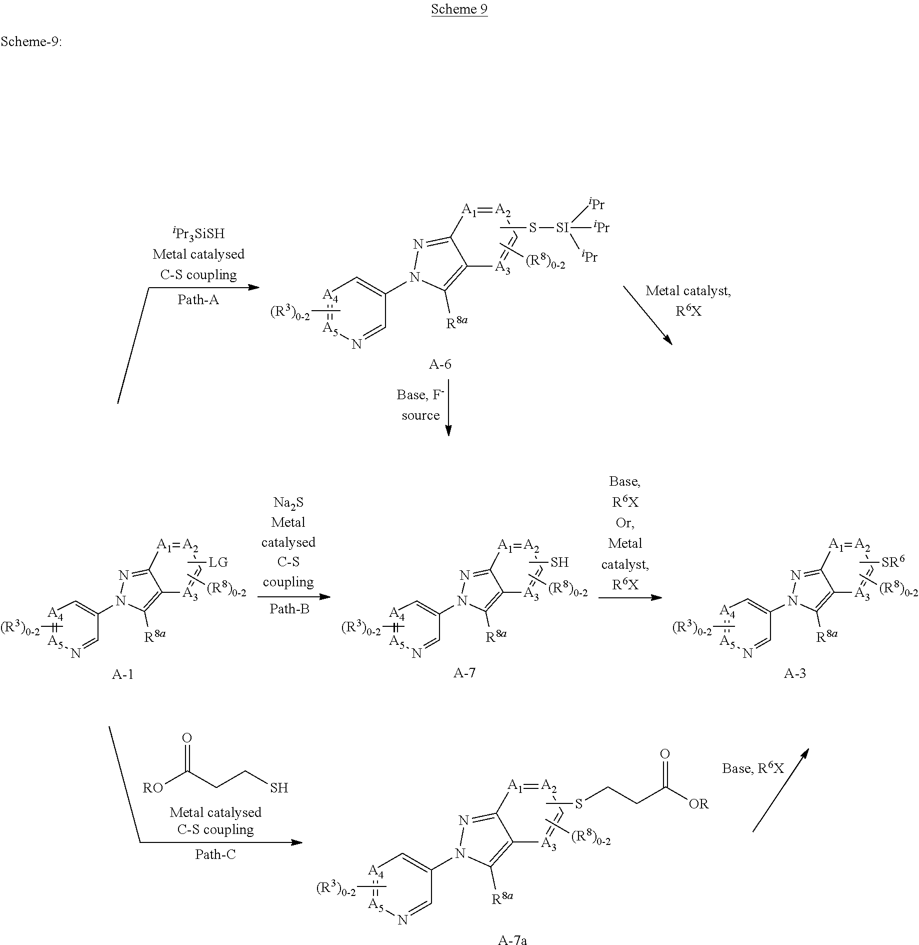

In a further embodiment of the present invention, the compounds of the formula A-3, wherein all substituents are as defined earlier, can be prepared by the methods given in the reaction scheme 9. Compounds of formula A-1 can undergo metal-catalyzed C—S coupling reactions as described above in a general protocol for the C—S coupling in the presence of trialkylylsilanethiols, such as triisopropylsilanethiol, to afford thiosilyl compounds of the formula A-6 (Path-A). Thiosilyls of formula A-6 can then be deprotected to afford compounds of formula A-7 by the use of a suitable base, such as, but not being limited to, sodium carbonate, potassium carbonate, cesium carbonate, or metal hydride bases such as, but not being limited to, sodium hydride, potassium hydride and fluoride sources such as metal fluorides, e.g., but not being limited to, of cesium fluoride, sodium fluoride or reagents in which the metal ion is exchanged by tetra-alkyl ammonium cations e.g., but not being limited to, as in tetramethylammonium fluoride, tetraethylammonium fluoride tetrabutylammonium fluoride, as described in the literature in WO2020257145 and in further literature cited therein. The thiols of formula A-7 can also be obtained via metal catalyzed coupling reactions between compounds of the formula A-1 and commercial sodium sulfide (Na₂S·9H₂O) using catalytic quantities of 1,2-ethanedithiol (Path-B; see, for example, Synlett 2017; 28(17) 2272-2276 and further literature cited therein). Thiols of formula A-7 can further be alkylated in the presence of suitable inorganic and organic bases and appropriate alkyl halides of the formula R⁶X to obtain compounds of the formula A-3. The alkyl halides of the formula R⁶X can be obtained commercially. Alternatively, the thiosilyl compounds of formula A-6 can be (hetero)arylated in the presence of appropriate (hetero)aryl halides, or triflates, or tosylates of the formula R⁶X using suitable metal catalyzed C—C cross-coupling reaction conditions to obtain the compounds of the formula A-3, as described in the literature in WO2011087712, WO2004087156 and in further literature cited therein. Furthermore, compounds of formula A-1 can undergo metal-catalyzed C—S coupling reactions in presence of mercapto esters as described in the general protocol above to afford sulfides of formula A-7a (Path-C), wherein, R-represent alkyl groups. A base mediated retro-Micheal reaction can be devised further to generate in situ metal sulfides that can undergo various S-alkylation with appropriate alkyl halides to attain a variety A-3 sulfides as described in the literature J. Med. Chem. 2020, 63, 2308-2324 and in the literature cited herein. Unless stated otherwise the definition of each variable is as defined above. X-represent leaving groups, such as halogens, triflates, mesylates or tosylates.

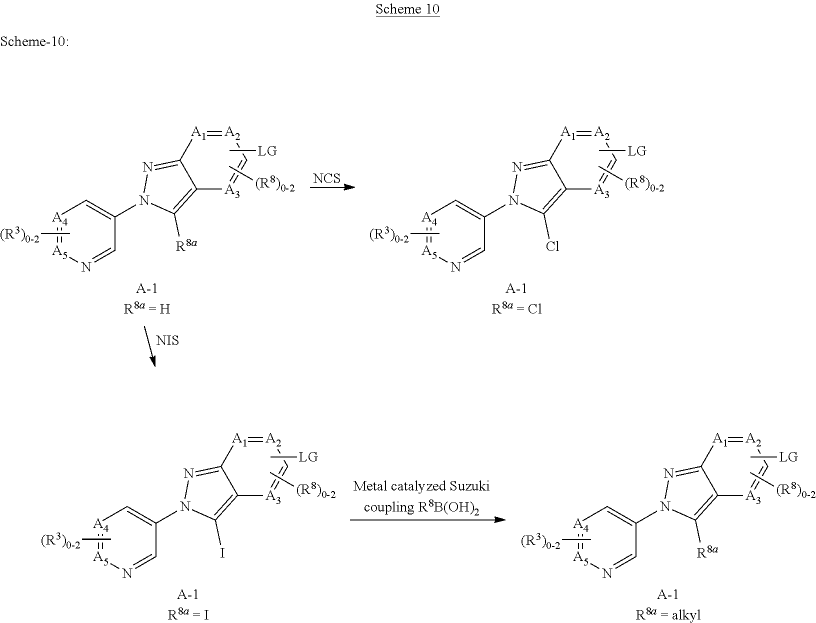

According to the scheme 10, the present invention provides a method for selective C—H functionalization at R⁸ of the central pyrazole ring of formula A-1, wherein the same R⁸ represents hydrogen and all the other variables carry their usual meaning. In presence of N-Chlorosuccinimide (NCS), A-1 can selectively undergo R⁸ chlorination at the central pyrazole ring to form compounds of the formula A-1, wherein the pyrazole-R⁸ represents Cl. Moreover, the same R⁸ hydrogen of A-1 can also be alkylated in two successive steps. First, a similar R⁸ iodination in presence of N-Iodosuccinimide, followed by a metal catalyzed Suzuki coupling reaction in presence of various alkyl boronic acids, represented by the formula R⁸B(OH)₂, to form compounds of formula A-1, wherein the pyrazole-R⁸ represents different alkyl groups (for a comprehensive overview on C—H functionalization at the Indazole R⁸, see: Org. Biomol. Chem., 2022, 20, 7746).

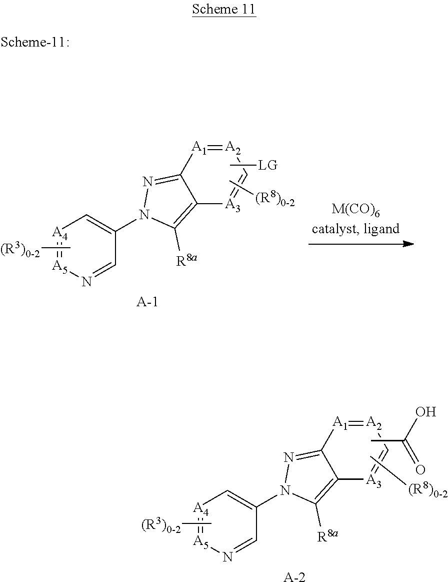

In a further embodiment according to the scheme 11, the present invention provides a method for the preparation of the compounds of formula A-2, wherein all substituents are as defined earlier. Compounds of the formula A-1, wherein A₁, A₂, A₃, A₄, A₅, R³, R⁸ and R^{8a} have their usual meaning as defined above and LG represents a leaving group, preferably a halogen, can be converted into carboxylic acids of the formula A-2, following a procedure as described in WO 2016144351 and in further literature cited therein. The metal hexacarbonyls that serve as “CO source” in this reaction are selected from, but not limited to, molybdenum hexacarbonyl and tungsten hexacarbonyl. The reaction is carried out in the presence of a base selected from, but not being limited to, metal hydrides, such as sodium hydride, lithium hydride, and potassium hydride; metal carbonates, such as sodium carbonate, potassium carbonate, cesium carbonate; organic amine bases, such as triethylamine, N,N-diisopropylethylamine, 1,8-diazabicyclo[5.4.0]undec-7-ene; and organic amines such as N,N-Diisopropylethylamine, and 1,8-diazabicyclo[5.4.0]undec-7-ene as the preferred bases. The catalysts that can be used for this reaction include for example, but are not limited to, organic palladium complexes such as a polymer-supported bis (acetato) triphenyl phosphine palladium (II), trans-di-μ-acetatobis[2-[bis(2-methylphenyl)phosphino]benzyl]dipalladium, and a polymer-carrying di(acetato) dicyclohexylphosphine palladium (II). Examples of ligands used for this reaction are selected from, but not limited to, tri-tert-butylphosphonium tetrafluoroborate. This reaction is typically carried out within a temperature range of 0-200° C., preferably 50-180° C. for a duration in the range of 30 minutes to 24 hours. The reaction can also be optionally performed under microwave irradiation conditions.

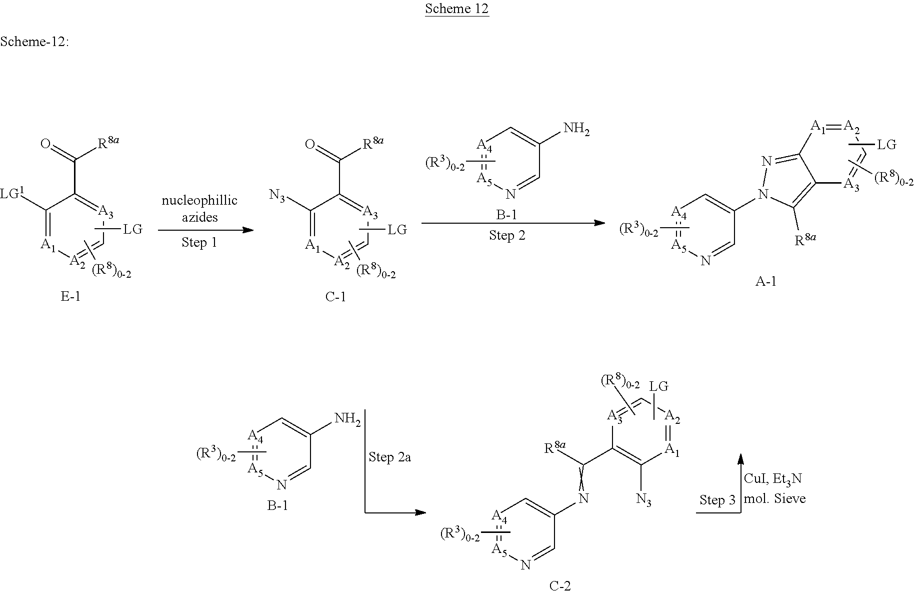

According to the scheme 12, the present invention provides a method for the preparation of compounds of formula A-1 from substituted benzaldehydes of formula E-1, wherein A₁, A₂, A₃, R⁸ and R^{8a} are as defined above. LG and LG¹ represent leaving groups, preferably halogen substituents. Suitably substituted benzaldehydes of the formula E-1 can undergo nucleophilic substitution reactions in presence of nucleophilic organic or inorganic azides, either at room temperature or at an elevated temperature to obtain aryl-azides of the formula C-1. The aryl-azides having formula C-1 can be further reacted with heteroaryl amines of formula B-1, wherein A₄, A₅, and R³ are as defined above, to form imine intermediates which undergo in situ cyclization at an elevated temperature to afford the desired bicyclic products of the formula A-1 (see, for example, WO2016144351). However, depending on the substrate nature, such elevated temperature in situ cyclization method may pose additional challenges. To circumvent this, a lower-temperature Cu/ligand mediated method can be implemented to cyclize C-2, as described in the literature Chem. Commun., 2011, 47, 10133-10135 and in the literature cited therein (Step-3).

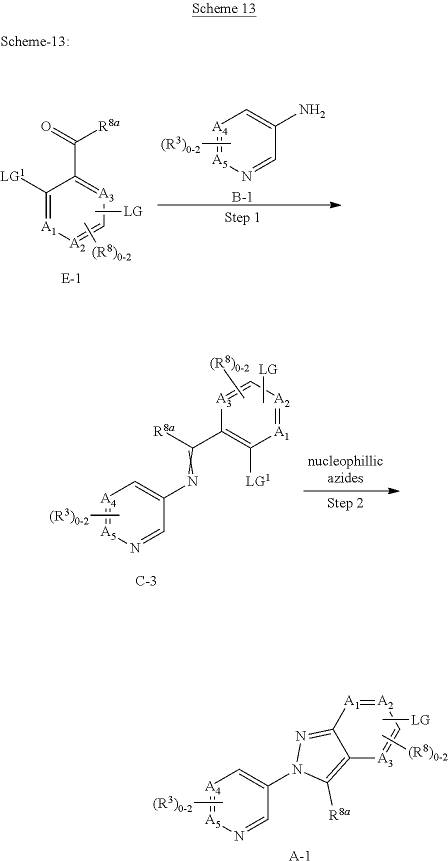

Alternatively, the compounds of formula A-1 can be prepared as described in the scheme 13. Substituted benzaldehydes of the formula E-1, wherein A₁, A₂, A₃, R⁸ and R^{8a} are as defined above, while LG and LG¹ represent a leaving group, preferably halogen substituents, are reacted with heteroaryl amines of the formula B-1, wherein A₄, A₅, and R³ are as defined above, either at room temperature or at an elevated temperature to form imines of the formula C-3. The imine of formula C-3, in the presence of a nucleophilic organic or inorganic azide, either at room temperature or at an elevated temperature, can first undergo an intermolecular substitution reaction followed by a consequent in situ intramolecular cyclization reaction to afford the bicyclic products of the formula A-1 (see, for example, WO2016144351).

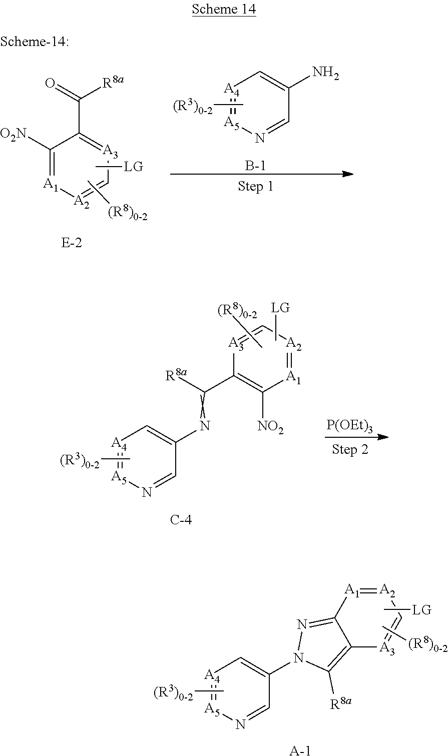

Alternatively, the compounds of formula A-1 can be prepared as described in the scheme 14. Suitably substituted 2-nitrobenzaldehydes of formula E-2, wherein A₁, A₂, A₃, R⁸ and R^{8a} are as defined above, can be reacted with heterocyclic amines of formula B-1, wherein A₄, A₅, and R³ are defined as above, to form imines C-4. Consequently, a reductive cyclization in the second reaction step, in presence of a suitable phosphorus (III) reagent, for example triethyl phosphite, at an elevated temperature, can afford bicyclic compounds of the formula A-1 (see, for example, US2019029788). Several alternate reductive cyclization methods are available and can be implemented as depicted in the Org. Lett. 2014, 16, 3114-3117 and in the literature cited therein.

In one embodiment, the present invention provides a method for the preparation of compounds of formula (I) wherein D is D₁ or D₃, comprising the steps of:
i. reacting a compound of formula (A-1) with a compound of formula (D₁-1) or (D₃-1) to obtain a compound of formula (I) wherein Z is a direct bond;

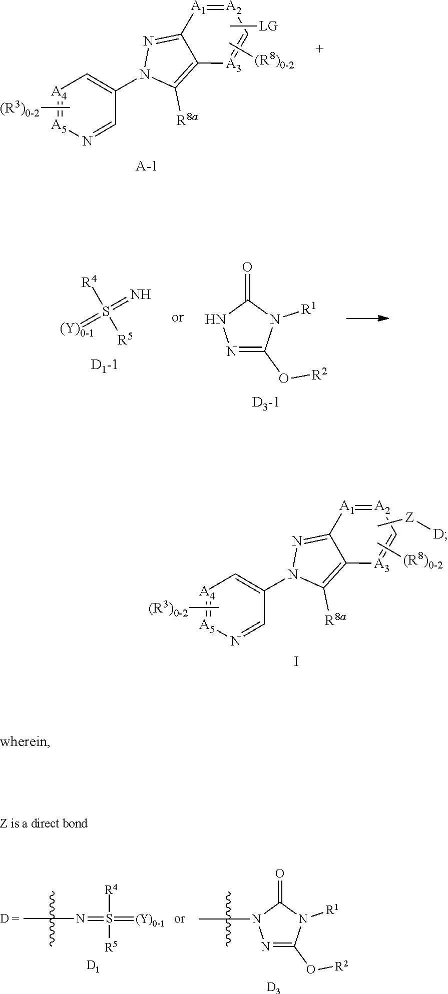

or
i. reacting a compound of formula (A-2) with a compound of formula (D₁-1) or (D₃-1) to obtain a compound of formula (I) wherein Z is a —C(═O)— bond;

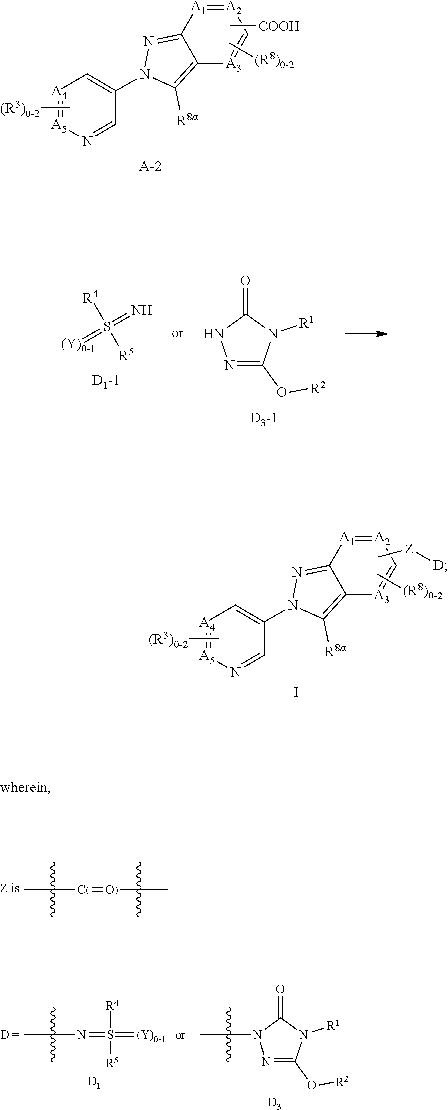

wherein, the definition of LG, A₁, A₂, A₃, A₄, A₅, R¹, R², R³, R⁴, R⁵, Rand R^{8a} are the same as defined in the description.

In another embodiment the present invention provides a method for the preparation of compounds of formula (I) wherein D is D₂, comprising the steps of:
i. reacting a compound of formula (A-1) with a compound of formula R⁶SH to obtain a compound of formula (A-3) wherein Z is a direct bond;

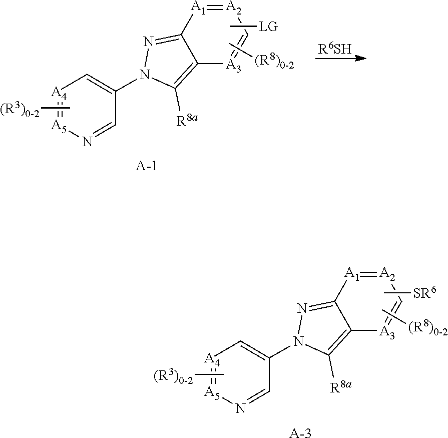

ii. converting the compound of formula (A-3) to a compound of formula (I) through O- and NH— transfer using a suitable oxidizing reagents and N-sources,

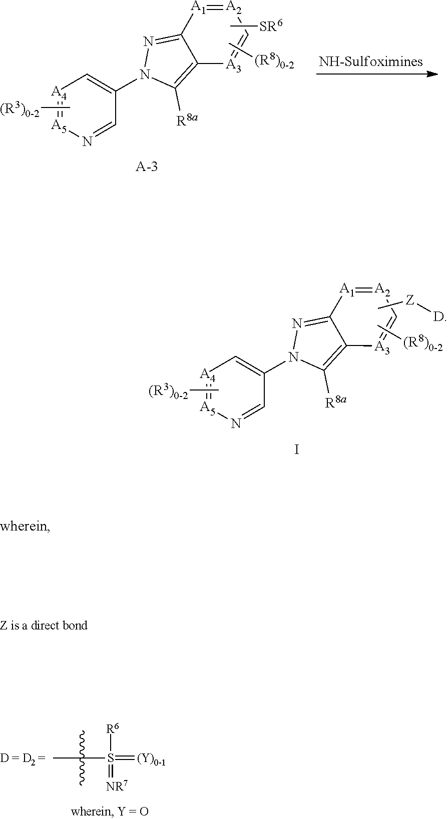

In one embodiment, the present invention provides a method for the preparation of compounds of formula (I) wherein D is D₂, and step ii can be carried out in single step as mentioned below:
ii. converting the compound of formula (A-3) to a compound of formula (I) through O- and NH-transfer using a suitable oxidizing reagents and N-sources,

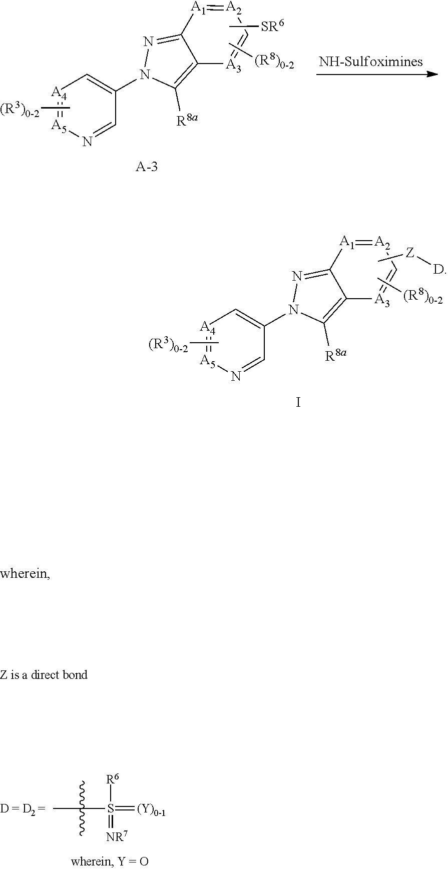

In one embodiment, the present invention provides a method for the preparation of compounds of formula (I) wherein D is D₂, and step ii can be carried out in two steps as mentioned below:
ii. oxidizing a compound of formula (A-3) using a suitable oxidizing reagent to obtain a compound of formula (A-4), which was further converted to compound of formula (I) by imination using a suitable N-source or imination reagent;
   or

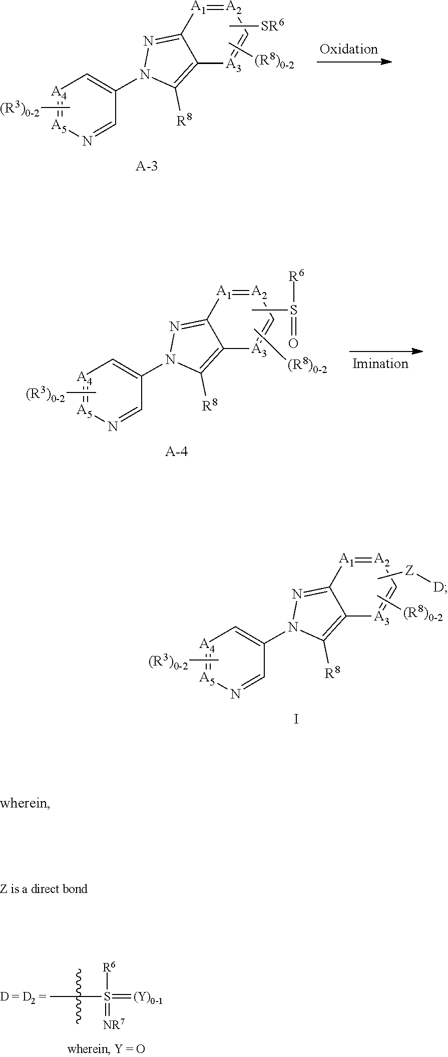

ii. imination of a compound of formula (A-3) using a suitable N-source or imination reagent to obtain a compound of formula (A-5a), which was further converted to compound of formula (I) by oxidation using a suitable oxidizing agent;

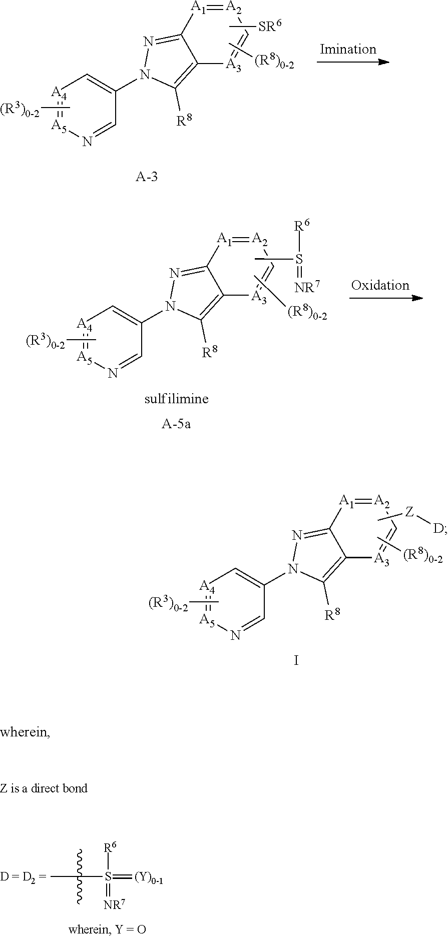

wherein, LG represents a leaving group, and A₁, A₂, A₃, A₄, A₅, R³, R⁶, R⁷, R⁸ and R^{8a} are same as defined in claim 1.

The suitable oxidizing agent in step (ii) is selected from: H₂O₂; H₂O₂, FeCl₃ (catalyst); H₂O₂, Cerium molybdenum oxide (catalyst); H₂O₂, Trifluoromethanesulfonic acid, Sodium tungsten oxide (Na₂WO₄); m-Chloroperbenzoic acid; Sodium periodate; phenyliodine(III) diacetate (PIDA); Hydrochloric acid or acetic acid, Hydrogen peroxide; Peracetic acid; Triphenylmethyl hydroperoxide, Catalysts: Bis(acetylacetonato)dioxomolybdenum; H₂O₂, Tetraphenylphosphonium bromide (PB-7-23-111′1′2)-Diaquaoxodiperoxymolybdenum1H-Imidazolium, 1,3-bis[(1S)-1-carboxy-2-methylpropyl]-, inner salt; 2-(1-Hydroperoxy-1-methylethyl)furan, Catalysts: Diethyl tartrate, Titanium isopropoxide; H₂O₂, Catalysts: Tantalum pentachloride; tert-Butyl hydroperoxide; Oxygen; Catalysts: Nitrogen dioxide; Oxygen; Catalysts: Nitrosonium tetrafluoroborate; tert-Butyl hypochlorite; Chlorotrimethylsilane, H₂O₂; Tetrabutylammonium nitrate, Catalyst: Cupric nitrate, tetracarbonylbis(triphenylphosphine) Molybdenum; or H₂O₂, Zirconium chloride (ZrCl₄). The suitable N-source or imination reagent in step ii is selected from:

Ammonium carbamate; Sodium azide, Sodium bicarbonate,

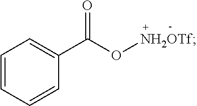

FeSO4, phenanthroline and MeCN (0.4 mL); sodium azide, conc. H₂SO₄; trifluoroacetamide, MgO, and Rh₂(OAc)₄, PhI(OAc)₂; (O-(mesitylsulfonyl)hydroxylamine); Ph-I=N-Ts (Ts=toluenesulfonyl); Ph-I=N-Ns (Ns=N-alkyl nitrobenzenesulfonamides); Ph-I=N-Ses (2-(Trimethylsilyl)ethanesulfonyl); or Rh₂(esp)₂ and O-(2,4-Dinitrophenyl)hydroxylamine.

In one embodiment the oxidising agent and N-source or imination reagent can be used in one pot or individually in a step wise reaction.

In one embodiment the oxidising agent and N-source or imination reagent can be used individually or in combination thereof.

Without further elaboration, it is reasonable to believe that any person skilled in the art who is using the preceding description can utilize the present invention to its fullest extent.

### CHEMISTRY EXAMPLES

The following examples set forth the manner and process of making compounds of the present invention without being a limitation thereof and include the best mode contemplated by the inventors for carrying out the invention.

### Experimental Procedure

### Example 1: Synthesis of isopropyl((2-(pyrimidin-5-yl)-2H-indazol-5-yl)imino)(3,3,3-trifluoropropyl)-λ⁶-sulfanone 101

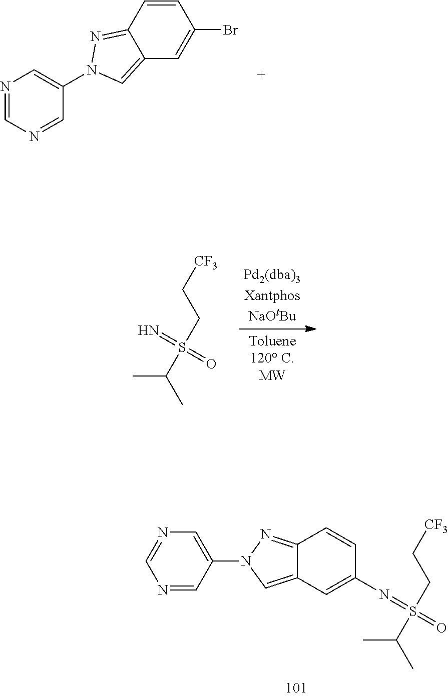 [SMILES: Brc1ccc2nn(-c3cncnc3)cc2c1 ; CC(C)S(=N)(=O)CCC(F)(F)F ; CC(C)S(=O)(CCC(F)(F)F)=Nc1ccc2nn(-c3cncnc3)cc2c1]

A solution of 5-bromo-2-(pyrimidin-5-yl)-2H-indazole (0.25 g, 0.909 mmol), imino(isopropyl)(3,3,3-trifluoropropyl)-λ⁶-sulfanone (0.185 g, 0.909 mmol) in toluene (5 mL) was treated with tris(dibenzylideneacetone)dipalladium(0) (Pd₂(dba)₃) (0.042 g, 0.045 mmol), (9,9-dimethyl-9H-xanthene-4,5-diyl)bis(diphenylphosphane) (Xantphos) (0.053 g, 0.091 mmol), sodium tert-butoxide (NaO^{t}Bu) (0.087 g, 0.909 mmol) and irradiated in a microwave device (MW) at 120° C. for 1 h. After completion of the reaction, the reaction mixture was quenched by the addition of water (10 mL) and extracted with ethyl acetate (2×25 mL). The combined organic layers were dried over anhydrous sodium sulfate and concentrated under reduced pressure to obtain an oily residue. This residue was purified by reverse phase preparative-high performance liquid chromatography (HPLC) (Gradient method: −10 mM ammonium acetate aqueous buffer:acetonitrile from 19:1 to 0:100) to obtain the desired isopropyl((2-(pyrimidin-5-yl)-2H-indazol-5-yl)imino)(3,3,3-trifluoropropyl)-λ⁶-sulfanone 101 (209 mg, 0.526 mmol, 58% yield).

TABLE 1
Representative compounds of the present disclosure were prepared according
to the suitable starting material and method as described in the Example 1.

| Comp | | |
|---|---|---|
| no. | Compound name | Analytical Data |
| 1 | dimethyl((2-(pyridin-3- | ¹H-NMR (400 MHz, DMSO-d₆) δ 9.27 (d, J = 2.2 Hz, 1H), |
| | yl)-2H-indazol-5- | 8.93 (d, J = 0.7 Hz, 1H), 8.60 (dd, J = 4.8, 1.3 Hz, 1H), 8.41 |
| | yl)imino)-λ⁶-sulfanone | (dq, J = 8.3, 1.4 Hz, 1H), 7.62-7.57 (m, 2H), 7.16 (q, J = 1.0 |
| | | Hz, 1H), 6.97 (dd, J = 9.2, 2.1 Hz, 1H), 3.23 (s, 6H); ESI MS |
| | | (m/z): 286.95 [M + 1] |
| 2 | ethyl(methyl)((2- | ¹H-NMR (400 MHz, DMSO-d₆) δ 9.27 (d, J = 2.1 Hz, 1H), |
| | (pyridin-3-yl)-2H- | 8.92 (d, J = 0.9 Hz, 1H), 8.60 (dd, J = 4.7, 1.4 Hz, 1H), 8.41 |
| | indazol-5-yl)imino)-λ⁶- | (dq, J = 8.3, 1.4 Hz, 1H), 7.62-7.56 (m, 2H), 7.17 (q, J = 0.9 |
| | sulfanone | Hz, 1H), 6.98 (dd, J = 9.2, 2.1 Hz, 1H), 3.37 (q, J = 7.3 Hz, |
| | | 2H), 3.12 (s, 3H), 1.29 (t, J = 7.3 Hz, 3H); ESI MS (m/z): |
| | | 300.9 [M + 1]. |
| 3 | diethyl((2-(pyridin-3- | ¹H-NMR (400 MHz, DMSO-d₆) δ 9.26 (d, J = 2.8 Hz, 1H), |
| | yl)-2H-indazol-5- | 8.91 (d, J = 0.9 Hz, 1H), 8.60 (dd, J = 4.7, 1.4 Hz, 1H), 8.41 |
| | yl)imino)-λ⁶-sulfanone | (dq, J = 8.4, 1.3 Hz, 1H), 7.61 (ddd, J = 8.3, 4.7, 0.7 Hz, 1H), |
| | | 7.56 (dt, J = 9.2, 0.9 Hz, 1H), 7.17 (q, J = 0.9 Hz, 1H), 7.00 |
| | | (dd, J = 9.2, 2.1 Hz, 1H), 3.34 (s, 2H), 3.29 (d, J = 7.3 Hz, |
| | | 2H), 1.26 (t, J = 7.3 Hz, 6H); ESI MS (m/z): 314.9 [M + 1]. |
| 4 | 1-((2-(pyridin-3-yl)-2H- | ¹H-NMR (400 MHz, DMSO-d₆) δ 9.27 (d, J = 2.8 Hz, 1H), |
| | indazol-5- | 8.94 (d, J = 0.9 Hz, 1H), 8.60 (dd, J = 4.6, 1.5 Hz, 1H), 8.41 |
| | yl)imino)tetrahydro- | (dq, J = 8.3, 1.4 Hz, 1H), 7.62-7.58 (m, 2H), 7.11 (q, J = 0.9 |
| | 1H-λ⁶-thiophene 1- | Hz, 1H), 6.98 (dd, J = 9.2, 2.1 Hz, 1H), 3.41-3.35 (m, 2H), |
| | oxide | 3.27-3.20 (m, 2H), 2.26-2.18 (m, 2H), 2.14-2.07 (m, 2H) |
| | | ESI MS (m/z): 312.9 [M + 1]. |
| 5 | ethyl((2-(5- | ¹H-NMR (400 MHz, DMSO-d₆) δ 9.20 (d, J = 1.2 Hz, 1H), |
| | fluoropyridin-3-yl)-2H- | 8.96 (d, J = 0.9 Hz, 1H), 8.63 (d, J = 2.4 Hz, 1H), 8.42 (dt, J = |
| | indazol-5- | 10.1, 2.3 Hz, 1H), 7.57 (d, J = 9.2 Hz, 1H), 7.15 (t, J = 0.9 Hz, |
| | yl)imino)(methyl)-λ⁶- | 1H), 7.00 (dd, J = 9.2, 1.8 Hz, 1H), 3.37 (q, J = 7.3 Hz, 2H), |
| | sulfanone | 3.13 (s, 3H), 1.29 (t, J = 7.3 Hz, 3H); ESI MS (m/z): 318.9 |
| | | [M + 1]. |
| 6 | diethyl((2-(5- | ¹H-NMR (400 MHz, DMSO-d₆) δ 9.19 (d, J = 1.5 Hz, 1H), |
| | fluoropyridin-3-yl)-2H- | 8.95 (d, J = 0.9 Hz, 1H), 8.63 (d, J = 2.8 Hz, 1H), 8.41 (dt, J = |
| | indazol-5-yl)imino)-λ⁶- | 10.2, 2.4 Hz, 1H), 7.56 (dd, J = 9.2, 0.9 Hz, 1H), 7.16 (q, J = |
| | sulfanone | 0.9 Hz, 1H), 7.02 (dd, J = 9.2, 2.1 Hz, 1H), 3.33 (d, J = 7.3 Hz, |
| | | 2H), 3.30 (d, J = 7.6 Hz, 2H), 1.25 (t, J = 7.5 Hz, 6H) |
| | | ESI MS (m/z): 332.85 [M + 1]. |
| 7 | 1-((2-(5-fluoropyridin- | ¹H-NMR (400 MHz, DMSO-d₆) δ 9.20 (t, J = 1.4 Hz, 1H), |
| | 3-yl)-2H-indazol-5- | 8.98 (d, J = 0.9 Hz, 1H), 8.63 (d, J = 2.4 Hz, 1H), 8.42 (dt, J = |
| | yl)imino)tetrahydro- | 10.1, 2.3 Hz, 1H), 7.59 (dd, J = 9.2, 0.9 Hz, 1H), 7.09 (q, J = |
| | 1H-λ⁶-thiophene 1- | 1.0 Hz, 1H), 7.00 (dd, J = 9.2, 2.1 Hz, 1H), 3.42-3.35 (m, 2H), |
| | oxide | 3.28-3.21 (m, 2H), 2.28-2.18 (m, 2H), 2.14-2.05 (m, 2H) |
| | | ESI MS (m/z): 330.85 [M + 1]. |
| 8 | ((2-(5-fluoropyridin-3- | ¹H-NMR (400 MHz, DMSO-d₆) δ 9.20 (d, J = 1.5 Hz, 1H), |
| | yl)-2H-indazol-5- | 8.97 (d, J = 0.9 Hz, 1H), 8.63 (d, J = 2.0 Hz, 1H), 8.42 (dt, J = |
| | yl)imino)dimethyl-λ⁶- | 10.1, 2.3 Hz, 1H), 7.57 (dt, J = 9.2, 0.9 Hz, 1H), 7.14 (q, J = |
| | sulfanone | 0.9 Hz, 1H), 6.98 (dd, J = 9.2, 2.1 Hz, 1H), 3.22 (d, J = 7.3 Hz, |
| | | 6H); ESI MS (m/z): 304.85 [M + 1]. |
| 9 | dimethyl((2-(pyrimidin- | ¹H-NMR (400 MHz, DMSO-d₆) δ 9.48 (s, 2H), 9.21 (s, 1H), |
| | 5-yl)-2H-indazol-5- | 8.99 (d, J = 0.9 Hz, 1H), 7.59 (dt, J = 9.2, 0.9 Hz, 1H), 7.16 (q, |
| | yl)imino)-λ⁶-sulfanone | J = 0.9 Hz, 1H), 6.99 (dd, J = 9.2, 2.1 Hz, 1H), 3.24 (s, 6H); |
| | | ESI MS (m/z): 287.85 [M + 1]. |
| 10 | ethyl(methyl)((2- | ¹H-NMR (400 MHz, DMSO-d₆) δ 9.47 (s, 2H), 9.21 (s, 1H), |
| | (pyrimidin-5-yl)-2H- | 8.98 (d, J = 0.9 Hz, 1H), 7.59 (d, J = 9.2 Hz, 1H), 7.17 (q, J = |
| | indazol-5-yl)imino)-λ⁶- | 0.9 Hz, 1H), 7.01 (dd, J = 9.2, 2.1 Hz, 1H), 3.38 (q, J = 7.3 Hz, |
| | sulfanone | 2H), 3.13 (s, 3H), 1.29 (t, J = 7.3 Hz, 3H); ESI MS (m/z): |
| | | 301.9 [M + 1]. |
| 11 | diethyl((2 -(pyrimidin-5- | ¹H-NMR (400 MHz, DMSO-d₆) δ 9.47 (s, 2H), 9.21 (s, 1H), |
| | yl)-2H-indazol-5- | 8.97 (d, J = 0.9 Hz, 1H), 7.58 (dt, J = 9.2, 0.9 Hz, 1H), 7.17 (q, |
| | yl)imino)-λ⁶-sulfanone | J = 0.9 Hz, 1H), 7.03 (dd, J = 9.2, 1.8 Hz, 1H), 3.35-3.29 (m, |
| | | 4H), 1.26 (t, J = 7.3 Hz, 6H); ESI MS (m/z): 315.9 [M + 1]. |
| 12 | 1-((2-(pyrimidin-5-yl)- | ¹H-NMR (400 MHz, DMSO-d₆) δ 9.48 (s, 2H), 9.21 (s, 1H), |
| | 2H-indazol-5- | 9.00 (d, J = 0.9 Hz, 1H), 7.61 (dt, J = 9.2, 0.9 Hz, 1H), 7.10 (q, |
| | yl)imino)tetrahydro- | J = 0.9 Hz, 1H), 7.01 (dd, J = 9.2, 2.1 Hz, 1H), 3.42-3.35 (m, |
| | 1H-λ⁶-thiophene 1- | 2H), 3.28-3.22 (m, 2H), 2.26-2.08 (m, 4H); ESI MS (m/z): |
| | oxide | 313.85 [M + 1]. |
| 13 | benzyl((2-(5- | ¹H-NMR (400 MHz, DMSO-d₆) δ 9.18 (s, 1H), 8.90 (s, 1H), |
| | fluoropyridin-3-yl)-2H- | 8.59 (d, J = 2.4 Hz, 1H), 8.36 (dt, J = 9.9, 2.3 Hz, 1H), 7.56 (d, |
| | indazol-5- | J = 9.2 Hz, 1H), 7.46-7.44 (m, 2H), 7.39-7.36 (m, 3H), 7.19 |
| | yl)imino)(methyl)-λ⁶- | (d, J = 1.5 Hz, 1H), 7.00 (dd, J = 9.3, 2.0 Hz, 1H), 4.74 (d, J = |
| | sulfanone | 13.8 Hz, 1H), 4.66 (d, J = 14.1 Hz, 1H), 3.08 (s, 3H) |
| | | ESI MS (m/z): 380[M + 1]. |
| 14 | ((2-(5-fluoropyridin-3- | ¹H-NMR (400 MHz, DMSO-d₆) δ 9.13 (d, J = 1.2 Hz, 1H), |
| | yl)-2H-indazol-5- | 8.88 (d, J = 1.0 Hz, 1H), 8.61-8.60 (m, 1H), 8.35 (dt, J = 10.2, |
| | yl)imino)(methyl)(phenyl)- | 2.3 Hz, 1H), 7.98-7.95 (m, 2H), 7.67-7.58 (m, 3H), 7.52 (dt, J = |
| | λ⁶-sulfanone | 9.2, 0.9 Hz, 1H), 7.04 (dd, J = 9.2, 2.1 Hz, 1H), 6.97 (q, J = |
| | | 0.9 Hz, 1H), 3.41 (s, 3H); ESI MS (m/z): 367 [M + 1]. |
| 15 | (4-fluorophenyl)((2-(5- | ¹H-NMR (400 MHz, DMSO-d₆) δ 9.13 (d, J = 1.5 Hz, 1H), |
| | fluoropyridin-3-yl)-2H- | 8.88 (d, J = 1.0 Hz, 1H), 8.61 (d, J = 2.2 Hz, 1H), 8.35 (dt, J = |
| | indazol-5- | 10.0, 2.3 Hz, 1H), 7.93-7.84 (m, 2H), 7.53 (dt, J = 9.1, 0.9 Hz, |
| | yl)imino)(isopropyl)-λ⁶- | 1H), 7.48-7.42 (m, 2H), 7.08-7.04 (m, 1H), 6.97 (q, J = 0.9 |
| | sulfanone | Hz, 1H), 3.70-3.59 (m, 1H), 1.33 (dd, J = 14.4, 6.6 Hz, 3H), |
| | | 1.18 (dd, J = 15.2, 6.8 Hz, 3H); ESI MS (m/z): 413.05 [M + 1]. |
| 16 | benzyl(methyl)((2- | ¹H-NMR (400 MHz, DMSO-d₆) δ 9.48 (s, 2H), 9.21 (s, 1H), |
| | (pyrimidin-5-yl)-2H- | 9.00 (d, J = 0.7 Hz, 1H), 7.60 (d, J = 9.0 Hz, 1H), 7.43-7.36 |
| | indazol-5-yl)imino)-λ⁶- | (m, 5H), 7.22 (q, J = 0.9 Hz, 1H), 6.98 (dd, J = 9.3, 2.0 Hz, |
| | sulfanone | 1H), 4.79 (d, J = 13.7 Hz, 1H), 4.70 (d, J = 13.7 Hz, 1H), 3.03 |
| | | (s, 3H); ESI MS (m/z): 364 [M + 1]. |
| 17 | methyl(phenyl)((2- | ¹H-NMR (400 MHz, DMSO-d₆) δ 9.41 (s, 2H), 9.19 (s, 1H), |
| | (pyrimidin-5-yl)-2H- | 8.90 (d, J = 1.0 Hz, 1H), 7.97-7.95 (m, 2H), 7.67-7.58 (m, |
| | indazol-5-yl)imino)-λ⁶- | 3H), 7.54 (dt, J = 9.2, 0.9 Hz, 1H), 7.05 (dd, J = 9.2, 2.1 Hz, |
| | sulfanone | 1H), 6.98 (q, J = 1.0 Hz, 1H), 3.42 (s, 3H); ESI MS (m/z): 350 |
| | | [M + 1]. |
| 18 | (4- | ¹H-NMR (400 MHz, DMSO-d₆) δ 9.41 (s, 2H), 9.19 (s, 1H), |
| | fluorophenyl)(isopropyl)((2- | 8.89 (d, J = 0.7 Hz, 1H), 7.92-7.89 (m, 2H), 7.55 (d, J = 9.3 |
| | (pyrimidin-5-yl)- | Hz, 1H), 7.45 (t, J = 8.8 Hz, 2H), 7.12-7.06 (m, 1H), 6.98-6.96 |
| | 2H-indazol-5- | (m, 1H), 3.70-3.60 (m, 1H), 1.36-1.32 (m, 3H), 1.20 (d, J = |
| | yl)imino)-λ⁶-sulfanone | 6.8 Hz, 3H); ESI MS (m/z): 396.05 [M + 1]. |
| 19 | tert-butyl(methyl)((2- | ¹H-NMR (400 MHz, DMSO-d₆) δ 9.47 (s, 2H), 9.21 (s, 1H), |
| | (pyrimidin-5-yl)-2H- | 8.99 (d, J = 0.7 Hz, 1H), 7.58 (dt, J = 9.2, 0.9 Hz, 1H), 7.18 (q, |
| | indazol-5-yl)imino)-λ⁶- | J = 0.9 Hz, 1H), 7.04 (dd, J = 9.3, 2.0 Hz, 1H), 3.01 (s, 3H), |
| | sulfanone | 1.44 (s, 9H); ESI MS (m/z): 330 [M + 1]. |
| 20 | benzyl(methyl)((2- | ¹H-NMR (400 MHz, DMSO-d₆) δ 9.27 (d, J = 2.4 Hz, 1H), |
| | (pyridin-3-yl)-2H- | 8.94 (d, J = 0.6 Hz, 1H), 8.60 (dd, J = 4.6, 1.5 Hz, 1H), 8.42 |
| | indazol-5-yl)imino)-6- | (dq, J = 8.3, 1.4 Hz, 1H), 7.63-7.58 (m, 2H), 7.43-7.35 (m, |
| | sulfanone | 5H), 7.22 (q, J = 0.9 Hz, 1H), 6.96 (dd, J = 9.2, 1.8 Hz, 1H), |
| | | 4.78 (d, J = 14.1 Hz, 1H), 4.69 (d, J = 13.8 Hz, 1H), 3.02 (s, |
| | | 3H); ESI MS (m/z): 363.05 [M + 1]. |
| 21 | methyl(phenyl)((2- | ¹H-NMR (400 MHz, DMSO-d₆) δ 9.20 (d, J = 2.1 Hz, 1H), |
| | (pyridin-3-yl)-2H- | 8.84 (d, J = 0.6 Hz, 1H), 8.58 (dd, J = 4.7, 1.4 Hz, 1H), 8.35 |
| | indazol-5-yl)imino)-λ⁶- | (dq, J = 8.3, 1.4 Hz, 1H), 7.98-7.95 (m, 2H), 7.67-7.56 (m, |
| | sulfanone | 4H), 7.53-7.51 (m, 1H), 7.02 (dd, J = 9.2, 1.8 Hz, 1H), 6.99 (q, |
| | | J = 1.0 Hz, 1H), 3.41 (s, 3H); ESI MS (m/z): 349.05 [M + 1]. |
| 22 | (3- | ¹H-NMR (400 MHz, DMSO-d₆) δ 9.21 (d, J = 2.4 Hz, 1H), |
| | fluorophenyl)(methyl)((2- | 8.87 (d, J = 0.9 Hz, 1H), 8.58 (dd, J = 4.7, 1.4 Hz, 1H), 8.35 |
| | (pyridin-3-yl)-2H- | (dq, J = 8.5, 1.4 Hz, 1H), 7.82-7.77 (m, 2H), 7.67 (td, J = 8.0, |
| | indazol-5-yl)imino)-λ⁶- | 5.6 Hz, 1H), 7.60-7.50 (m, 3H), 7.05-7.01 (m, 2H), 3.47 (s, |
| | sulfanone | 3H); ESI MS (m/z): 367.05 [M + 1]. |
| 23 | methyl((2-(pyridin-3- | ¹H-NMR (400 MHz, DMSO-d₆) δ 9.20 (d, J = 2.1 Hz, 1H), |
| | yl)-2H-indazol-5- | 8.84 (d, J = 0.9 Hz, 1H), 8.58 (dd, J = 4.6, 1.5 Hz, 1H), 8.35 |
| | yl)imino)(p-tolyl)-λ⁶- | (dq, J = 8.3, 1.4 Hz, 1H), 7.83 (d, J = 8.6 Hz, 2H), 7.59-7.56 |
| | sulfanone | (m, 1H), 7.52 (d, J = 9.2 Hz, 1H), 7.41-7.35 (m, 2H), 7.32- |
| | | 7.09 (m, 1H), 7.05-7.00 (m, 1H), 6.97 (q, J = 0.9 Hz, 1H), 3.37 |
| | | (s, 3H), 2.36 (d, J = 17.1 Hz, 3H); ESI MS (m/z): 363.05 |
| | | [M + 1]. |
| 24 | ethyl(phenyl)((2- | ¹H-NMR (400 MHz, DMSO-d₆) δ 9.20 (d, J = 2.4 Hz, 1H), |
| | (pyridin-3-yl)-2H- | 8.84 (s, 1H), 8.57 (d, J = 3.7 Hz, 1H), 8.35 (d, J = 8.3 Hz, 1H), |
| | indazol-5-yl)imino)-λ⁶- | 7.90 (d, J = 7.0 Hz, 2H), 7.68-7.51 (m, 5H), 7.05 (dd, J = 9.2, |
| | sulfanone | 1.8 Hz, 1H), 7.00 (s, 1H), 3.56-3.46 (m, 2H), 1.17 (t, J = 7.3 |
| | | Hz, 3H); ESI MS (m/z): 363.05 [M + 1]. |
| 25 | tert-butyl(methyl)((2- | ¹H-NMR (400 MHz, DMSO-d₆) δ 9.27 (d, J = 2.2 Hz, 1H), |
| | (pyridin-3-yl)-2H- | 8.92 (d, J = 1.0 Hz, 1H), 8.60 (dd, J = 4.6, 1.5 Hz, 1H), 8.41 |
| | indazol-5-yl)imino)-λ⁶- | (dq, J = 8.3, 1.4 Hz, 1H), 7.61 (ddd, J = 8.3, 4.6, 0.7 Hz, 1H), |
| | sulfanone | 7.56 (dt, J = 9.1, 0.9 Hz, 1H), 7.18 (q, J = 0.9 Hz, 1H), 7.01 |
| | | (dd, J = 9.0, 2.0 Hz, 1H), 3.00 (s, 3H), 1.44 (s, 9H) |
| | | ESI MS (m/z): 363.05 [M + 1]. |
| 26 | (4- | ¹H-NMR (400 MHz, DMSO-d₆) δ 9.21 (d, J = 2.2 Hz, 1H), |
| | chlorophenyl)(methyl)((2- | 8.85 (d, J = 0.7 Hz, 1H), 8.58 (dd, J = 4.8, 1.3 Hz, 1H), 8.35 |
| | (pyridin-3-yl)-2H- | (dq, J = 8.4, 1.3 Hz, 1H), 7.96 (dt, J = 9.1, 2.2 Hz, 2H), 7.68 |
| | indazol-5-yl)imino)-λ⁶- | (dt, J = 9.1, 2.3 Hz, 2H), 7.60-7.52 (m, 2H), 7.03-6.98 (m, |
| | sulfanone | 2H), 3.44 (s, 3H); ESI MS (m/z): 382.95 [M + 1]. |
| 27 | methyl(pentyl)((2- | ¹H-NMR (400 MHz, DMSO-d₆) δ 9.27 (d, J = 2.2 Hz, 1H), |
| | (pyridin-3-yl)-2H- | 8.92 (d, J = 1.0 Hz, 1H), 8.60 (dd, J = 4.8, 1.3 Hz, 1H), 8.41 |
| | indazol-5-yl)imino)-λ⁶- | (dq, J = 8.3, 1.4 Hz, 1H), 7.61 (ddd, J = 8.3, 4.8, 0.6 Hz, 1H), |
| | sulfanone | 7.57 (dt, J = 9.2, 0.9 Hz, 1H), 7.16 (q, J = 1.0 Hz, 1H), 6.97 |
| | | (dd, J = 9.2, 2.1 Hz, 1H), 3.34 (dd, J = 9.9, 6.0 Hz, 2H), 3.13 |
| | | (s, 3H), 1.80-1.72 (m, 2H), 1.39-1.24 (m, 4H), 0.85 (t, J = 7.2 |
| | | Hz, 3H); ESI MS (m/z): 342.6 [M + 1]. |
| 28 | diisopropyl((2-(pyridin- | ¹H-NMR (400 MHz, DMSO-d₆) δ 9.26-9.20 (m, 1H), 8.89 (d, |
| | 3-yl)-2H-indazol-5- | J = 0.7 Hz, 1H), 8.59 (dd, J = 4.8, 1.3 Hz, 1H), 8.40 (dq, J = |
| | yl)imino)-λ⁶-sulfanone | 8.3, 1.4 Hz, 1H), 7.60 (ddd, J = 8.3, 4.6, 0.7 Hz, 1H), 7.54- |
| | | 7.52 (m, 1H), 7.20 (q, J = 0.9 Hz, 1H), 7.09 (dd, J = 9.2, 2.1 |
| | | Hz, 1H), 3.66-3.55 (m, 2H), 1.34 (d, J = 6.6 Hz, 6H), 1.27 (d, |
| | | J = 6.8 Hz, 6H); ESI MS (m/z): 342.55 [M + 1]. |
| 29 | ethyl(isopropyl)((2- | ¹H-NMR (400 MHz, DMSO-d₆) δ 9.26 (d, J = 2.2 Hz, 1H), |
| | (pyridin-3-yl)-2H- | 8.90 (d, J = 1.0 Hz, 1H), 8.60 (dd, J = 4.6, 1.5 Hz, 1H), 8.40 |
| | indazol-5-yl)imino)-λ⁶- | (dq, J = 8.4, 1.3 Hz, 1H), 7.60 (ddd, J = 8.4, 4.8, 0.7 Hz, 1H), |
| | sulfanone | 7.57-7.54 (m, 1H), 7.18 (q, J = 1.0 Hz, 1H), 7.03 (dd, J = 9.0, |
| | | 2.0 Hz, 1H), 3.57-3.50 (m, 1H), 3.27 (t, J = 7.3 Hz, 2H), 1.37 |
| | | (d, J = 6.8 Hz, 3H), 1.30 (d, J = 6.8 Hz, 3H), 1.24 (t, J = 7.5 |
| | | Hz, 3H); ESI MS (m/z): 328.55 [M + 1]. |
| 30 | methyl(pyridin-3- | ¹H-NMR (400 MHz, DMSO-d₆) δ 9.21 (d, J = 2.2 Hz, 1H), |
| | yl)((2-(pyridin-3-yl)- | 9.09 (dd, J = 2.3, 0.6 Hz, 1H), 8.87 (d, J = 0.7 Hz, 1H), 8.80 |
| | 2H-indazol-5- | (dd, J = 4.9, 1.5 Hz, 1H), 8.58 (dd, J = 4.6, 1.5 Hz, 1H), 8.34 |
| | yl)imino)-λ⁶-sulfanone | (tdd, J = 8.1, 2.5, 1.6 Hz, 2H), 7.64 (ddd, J = 8.1, 4.8, 0.9 Hz, |
| | | 1H), 7.60-7.53 (m, 2H), 7.05-7.02 (m, 2H), 3.53 (s, 3H) |
| | | ESI MS (m/z): 349.6 [M + 1]. |
| 31 | diisopropyl((2- | ¹H-NMR (400 MHz, DMSO-d₆) δ 9.46 (s, 2H), 9.20 (s, 1H), |
| | (pyrimidin-5-yl)-2H- | 8.95 (d, J = 1.0 Hz, 1H), 7.56-7.54 (m, 1H), 7.56-7.10 (m, |
| | indazol-5-yl)imino)-λ⁶- | 2H), 7.20 (q, J = 0.9 Hz, 1H), 7.11 (dd, J = 9.3, 2.0 Hz, 1H), |
| | sulfanone | 3.65-3.58 (m, 2H), 1.30 (dd, J = 27.0, 6.7 Hz, 12H); ESI MS |
| | | (m/z): 343.65 [M + 1]. |
| 32 | ethyl(isopropyl)((2- | ¹H-NMR (400 MHz, DMSO-d₆) δ 9.47 (s, 2H), 9.21 (t, J = 4.8 |
| | (pyrimidin-5-yl)-2H- | Hz, 1H), 8.97 (d, J = 1.0 Hz, 1H), 7.59-7.56 (m, 1H), 7.18 (q, |
| | indazol-5-yl)imino)-λ⁶- | J = 0.9 Hz, 1H), 7.05 (dd, J = 9.2, 2.1 Hz, 1H), 3.58-3.51 (m, |
| | sulfanone | 1H), 3.28 (t, J = 7.3 Hz, 2H), 1.37 (d, J = 6.8 Hz, 3H), 1.30 (d, |
| | | J = 6.8 Hz, 3H), 1.24 (t, J = 7.3 Hz, 3H); ESI MS (m/z): 329.6 |
| | | [M + 1]. |
| 33 | methyl(pentyl)((2- | ¹H-NMR (400 MHz, DMSO-d₆) δ 9.47 (s, 2H), 9.21 (s, 1H), |
| | (pyrimidin-5-yl)-2H- | 8.99 (d, J = 1.0 Hz, 1H), 7.59 (dt, J = 9.2, 0.9 Hz, 1H), 7.16 (q, |
| | indazol-5-yl)imino)-λ⁶- | J = 1.0 Hz, 1H), 7.00 (dd, J = 9.0, 2.0 Hz, 1H), 3.36 (dd, J = |
| | sulfanone | 10.0, 6.1 Hz, 2H), 3.15 (s, 3H), 1.80-1.72 (m, 2H), 1.38-1.26 |
| | | (m, 4H), 0.84 (t, J = 7.1 Hz, 3H); ESI MS (m/z): 343.6 [M + 1]. |
| 34 | ethyl(phenyl)((2- | ¹H-NMR (400 MHz, DMSO-d₆) δ 9.40 (s, 2H), 9.19 (s, 1H), |
| | (pyrimidin-5-yl)-2H- | 8.90 (d, J = 1.0 Hz, 1H), 7.92-7.89 (m, 2H), 7.69-7.59 (m, |
| | indazol-5-yl)imino)-λ⁶- | 3H), 7.55 (dt, J = 9.2, 0.9 Hz, 1H), 7.07 (dd, J = 9.3, 2.0 Hz, |
| | sulfanone | 1H), 6.99 (q, J = 0.9 Hz, 1H), 3.57-3.47 (m, 2H), 1.17 (t, J = |
| | | 7.3 Hz, 3H); ESI MS (m/z): 363.6 [M + 1]. |
| 35 | methyl((2-(pyrimidin-5- | ¹H-NMR (400 MHz, DMSO-d₆) δ 9.41 (s, 2H), 9.19 (s, 1H), |
| | yl)-2H-indazol-5- | 8.90 (d, J = 0.7 Hz, 1H), 7.83 (d, J = 8.3 Hz, 2H), 7.55-7.52 |
| | yl)imino)(p-tolyl)-λ⁶- | (m, 1H), 7.40 (d, J = 8.1 Hz, 2H), 7.03 (dd, J = 9.3, 2.0 Hz, |
| | sulfanone | 1H), 6.96 (q, J = 0.9 Hz, 1H), 3.38 (s, 3H), 2.34 (s, 3H); ESI |
| | | MS (m/z): 363.6 [M + 1]. |
| 36 | (3- | ¹H-NMR (400 MHz, DMSO-d₆) δ 9.41 (s, 2H), 9.19 (s, 1H), |
| | fluorophenyl)(methyl)((2- | 8.93 (d, J = 1.0 Hz, 1H), 7.82-7.77 (m, 2H), 7.69-7.64 (m, |
| | (pyrimidin-5-yl)-2H- | 1H), 7.57-7.52 (m, 2H), 7.06-7.00 (m, 2H), 3.48 (s, 3H) |
| | indazol-5-yl)imino)-λ⁶- | ESI MS (m/z): 367.6 [M + 1]. |
| | sulfanone | |
| 37 | (4- | ¹H-NMR (400 MHz, DMSO-d₆) δ 9.41 (s, 2H), 9.19 (s, 1H), |
| | chlorophenyl)(methyl)((2- | 8.92 (d, J = 0.7 Hz, 1H), 7.96 (dt, J = 9.1, 2.3 Hz, 2H), 7.69 |
| | (pyrimidin-5-yl)-2H- | (dt, J = 9.1, 2.3 Hz, 2H), 7.57-7.54 (m, 1H), 7.03 (dd, J = 9.3, |
| | indazol-5-yl)imino)-λ⁶- | 2.0 Hz, 1H), 6.98 (q, J = 0.9 Hz, 1H), 3.45 (s, 3H); ESI MS |
| | sulfanone | (m/z): 383.7 [M + 1]. |
| 38 | (4- | ¹H-NMR (400 MHz, DMSO-d₆) δ 9.20 (d, J = 2.2 Hz, 1H), |
| | fluorophenyl)(isopropyl)((2- | 8.84 (d, J = 1.0 Hz, 1H), 8.58 (dd, J = 4.6, 1.5 Hz, 1H), 8.35 |
| | (pyridin-3-yl)-2H- | (dq, J = 8.5, 1.3 Hz, 1H), 7.93-7.88 (m, 2H), 7.57 (ddd, J = |
| | indazol-5-yl)imino)-λ⁶- | 8.3, 4.6, 0.7 Hz, 1H), 7.54-7.52 (m, 1H), 7.48-7.42 (m, 2H), |
| | sulfanone | 7.06 (dd, J = 9.0, 2.0 Hz, 1H), 6.99 (q, J = 0.9 Hz, 1H), 3.67- |
| | | 3.60 (m, 1H), 1.35 (d, J = 6.8 Hz, 3H), 1.19 (d, J = 6.6 Hz, |
| | | 3H); ESI MS (m/z): 395.25 [M + 1]. |
| 39 | methyl(pyridin-3- | ¹H-NMR (400 MHz, DMSO-d₆) δ 9.43 (s, 2H), 9.21 (s, 1H), |
| | yl)((2-(pyrimidin-5-yl)- | 9.10 (d, J = 1.7 Hz, 1H), 8.94 (d, J = 0.7 Hz, 1H), 8.82 (dd, J = |
| | 2H-indazol-5- | 4.9, 1.5 Hz, 1H), 8.35 (dt, J = 8.1, 2.0 Hz, 1H), 7.66 (dd, J = |
| | yl)imino)-λ⁶-sulfanone | 7.7, 4.5 Hz, 1H), 7.58 (d, J = 9.3 Hz, 1H), 7.09-7.05 (m, 2H), |
| | | 3.55 (s, 3H); ESI MS (m/z): 351.05 [M + 1]. |
| 40 | ((2-(5-fluoropyridin-3- | ¹H-NMR (400 MHz, DMSO-d₆) δ 9.13 (d, J = 1.2 Hz, 1H), |
| | yl)-2H-indazol-5- | 9.09 (dd, J = 2.4, 0.7 Hz, 1H), 8.90 (d, J = 0.7 Hz, 1H), 8.80 |
| | yl)imino)(methyl)(pyridin- | (dd, J = 4.8, 1.6 Hz, 1H), 8.61 (d, J = 2.4 Hz, 1H), 8.37-8.32 |
| | 3-yl)-λ⁶-sulfanone | (m, 2H), 7.64 (ddd, J = 8.1, 4.8, 0.9 Hz, 1H), 7.56-7.53 (m, |
| | | 1H), 7.06-7.01 (m, 2H), 3.53 (s, 3H); ESI MS (m/z): 368.10 |
| | | [M + 1]. |
| 41 | ethyl((2-(5- | ¹H-NMR (400 MHz, DMSO-d₆) δ 9.19 (s, 1H), 8.95 (d, J = |
| | fluoropyridin-3-yl)-2H- | 0.7 Hz, 1H), 8.63 (d, J = 2.4 Hz, 1H), 8.41 (dt, J = 10.0, 2.3 |
| | indazol-5- | Hz, 1H), 7.55 (d, J = 9.0 Hz, 1H), 7.16 (q, J = 0.9 Hz, 1H), |
| | yl)imino)(isopropyl)-λ⁶- | 7.04 (dd, J = 9.0, 2.0 Hz, 1H), 3.54 (td, J = 13.7, 6.8 Hz, 1H), |
| | sulfanone | 3.28 (t, J = 7.3 Hz, 2H), 1.37 (dd, J = 15.2, 8.3 Hz, 3H), 1.32- |
| | | 1.28 (m, 3H), 1.26-1.19 (m, 3H); ESI MS (m/z): 347.10 |
| | | M + 1]. |
| 42 | ((2-(5-fluoropyridin-3- | ¹H-NMR (400 MHz, DMSO-d₆) δ 9.20 (d, J = 1.2 Hz, 1H), |
| | yl)-2H-indazol-5- | 8.96 (d, J = 1.0 Hz, 1H), 8.63 (d, J = 2.9 Hz, 1H), 8.42 (dt, J = |
| | yl)imino)(methyl)(pentyl)- | 10.0, 2.3 Hz, 1H), 7.57 (dt, J = 9.1, 0.9 Hz, 1H), 7.14 (q, J = |
| | λ⁶-sulfanone | 0.9 Hz, 1H), 6.99 (dd, J = 9.2, 2.1 Hz, 1H), 3.35 (dd, J = 9.8, |
| | | 6.1 Hz, 2H), 3.14 (s, 3H), 1.80-1.72 (m, 2H), 1.38-1.26 (m, |
| | | 4H), 0.84 (t, J = 7.1 Hz, 3H); ESI MS (m/z): 361.10 [M + 1]. |
| 43 | diethyl((2-(pyridazin-4- | ¹H-NMR (400 MHz, DMSO-d₆) δ 9.99 (q, J = 1.2 Hz, 1H), |
| | yl)-2H-indazol-5- | 9.36 (dd, J = 5.9, 1.0 Hz, 1H), 9.11 (d, J = 1.0 Hz, 1H), 8.26 |
| | yl)imino)-λ⁶-sulfanone | (q, J = 2.9 Hz, 1H), 7.58 (d, J = 9.3 Hz, 1H), 7.14 (q, J = 0.9 |
| | | Hz, 1H), 7.04 (dd, J = 9.2, 2.1 Hz, 1H), 3.35 (d, J = 7.3 Hz, |
| | | 3H), 3.30 (s, 1H), 1.25 (q, J = 7.5 Hz, 6H); ESI MS (m/z): |
| | | 316.05 [M + 1]. |
| 44 | benzyl(methyl)((2- | ¹H-NMR (400 MHz, DMSO-d₆) δ 10.00 (dd, J = 2.9, 1.0 Hz, |
| | (pyridazin-4-yl)-2H- | 1H), 9.36 (dd, J = 5.7, 0.9 Hz, 1H), 9.13 (d, J = 1.0 Hz, 1H), |
| | indazol-5-yl)imino)-λ⁶- | 8.27 (q, J = 2.9 Hz, 1H), 7.60 (d, J = 9.3 Hz, 1H), 7.43-7.36 |
| | sulfanone | (m, 5H), 7.18 (q, J = 0.9 Hz, 1H), 6.99 (dd, J = 9.2, 2.1 Hz, |
| | | 1H), 4.80 (d, J = 13.7 Hz, 1H), 4.72 (d, J = 13.7 Hz, 1H), 3.04 |
| | | (s, 3H); ESI MS (m/z): 364.10 [M + 1]. |
| 45 | methyl(phenyl)((2- | ¹H-NMR (400 MHz, DMSO-d₆) δ 9.92 (dd, J = 2.9, 1.0 Hz, |
| | (pyridazin-4-yl)-2H- | 1H), 9.33 (dd, J = 5.9, 1.0 Hz, 1H), 9.04 (d, J = 1.0 Hz, 1H), |
| | indazol-5-yl)imino)-λ⁶- | 8.20 (q, J = 2.9 Hz, 1H), 7.96 (dt, J = 6.6, 1.7 Hz, 2H), 7.68- |
| | sulfanone | 7.59 (m, 3H), 7.54 (d, J = 9.3 Hz, 1H), 7.06 (dd, J = 9.3, 2.0 |
| | | Hz, 1H), 6.94 (q, J = 0.9 Hz, 1H), 3.43 (s, 3H); ESI MS (m/z): |
| | | 350.15 [M + 1]. |
| 46 | (4- | ¹H-NMR (400 MHz, DMSO-d₆) δ 9.92 (dd, J = 2.9, 1.0 Hz, |
| | fluorophenyl)(isopropyl)((2- | 1H), 9.33 (dd, J = 5.9, 1.0 Hz, 1H), 9.03 (d, J = 1.0 Hz, 1H), |
| | (pyridazin-4-yl)- | 8.20 (q, J = 2.9 Hz, 1H), 7.92-7.87 (m, 2H), 7.55 (d, J = 9.3 |
| | 2H-indazol-5- | Hz, 1H), 7.48-7.41 (m, 2H), 7.10 (dd, J = 9.2, 2.1 Hz, 1H), |
| | yl)imino)-λ⁶-sulfanone | 6.94 (q, J = 0.9 Hz, 1H), 3.69-3.63 (m, 1H), 1.36-1.32 (m, |
| | | 3H), 1.21-1.17 (m, 3H); ESI MS (m/z): 396.20 [M + 1]. |
| 47 | methyl(pentyl)((2- | ¹H-NMR (400 MHz, DMSO-d₆) δ 9.99 (q, J = 1.2 Hz, 1H), |
| | (pyridazin-4-yl)-2H- | 9.36 (dd, J = 5.9, 1.0 Hz, 1H), 9.11 (d, J = 1.0 Hz, 1H), 8.27 |
| | indazol-5-yl)imino)-λ⁶- | (q, J = 2.9 Hz, 1H), 7.60-7.57 (m, 1H), 7.13 (q, J = 1.0 Hz, |
| | sulfanone | 1H), 7.01 (dd, J = 9.3, 2.0 Hz, 1H), 3.37 (dd, J = 10.1, 5.7 Hz, |
| | | 2H), 3.16 (s, 3H), 1.79-1.72 (m, 2H), 1.38-1.26 (m, 4H), 0.84 |
| | | (t, J = 7.1 Hz, 3H); ESI MS (m/z): 344.20 [M + 1]. |
| 48 | ethyl(isopropyl)((2- | ¹H-NMR (400 MHz, DMSO-d₆) δ 9.99 (dd, J = 2.9, 1.0 Hz, |
| | (pyridazin-4-yl)-2H- | 1H), 9.36 (dd, J = 5.9, 1.0 Hz, 1H), 9.10 (d, J = 1.0 Hz, 1H), |
| | indazol-5-yl)imino)-λ⁶- | 8.26 (q, J = 2.9 Hz, 1H), 7.57 (d, J = 9.3 Hz, 1H), 7.15 (q, J = |
| | sulfanone | 1.0 Hz, 1H), 7.07 (dd, J = 9.3, 2.0 Hz, 1H), 3.59-3.52 (m, 1H), |
| | | 3.33-3.29 (m, 2H), 1.37 (d, J = 6.8 Hz, 3H), 1.30 (d, J = 6.8 |
| | | Hz, 3H), 1.24 (t, J = 7.3 Hz, 3H); ESI MS (m/z): 330.20 |
| | | [M + 1]. |
| 49 | methyl((2-(pyridazin-4- | ¹H-NMR (400 MHz, DMSO-d₆) δ 9.93 (dd, J = 2.9, 1.0 Hz, |
| | yl)-2H-indazol-5- | 1H), 9.34 (dd, J = 5.9, 1.0 Hz, 1H), 9.09 (dd, J = 2.3, 0.6 Hz, |
| | yl)imino)(pyridin-3-yl)- | 1H), 9.06 (d, J = 1.0 Hz, 1H), 8.81 (dd, J = 4.8, 1.6 Hz, 1H), |
| | λ⁶-sulfanone | 8.34 (ddd, J = 8.1, 2.3, 1.6 Hz, 1H), 8.21 (q, J = 2.9 Hz, 1H), |
| | | 7.65 (ddd, J = 8.1, 4.9, 0.7 Hz, 1H), 7.57 (d, J = 9.0 Hz, 1H), |
| | | 7.07 (dd, J = 9.3, 2.0 Hz, 1H), 6.99 (q, J = 0.9 Hz, 1H), 3.54 |
| | | (s, 3H); ESI MS (m/z): 351.10 [M + 1]. |
| 50 | (4- | ¹H-NMR (400 MHz, DMSO-d₆) δ 9.93 (dd, J = 2.9, 1.0 Hz, |
| | chlorophenyl)(methyl)((2- | 1H), 9.34 (dd, J = 5.9, 1.0 Hz, 1H), 9.05 (d, J = 0.7 Hz, 1H), |
| | (pyridazin-4-yl)-2H- | 8.21 (q, J = 2.9 Hz, 1H), 7.96 (dt, J = 9.0, 2.3 Hz, 2H), 7.69 |
| | indazol-5-yl)imino)-λ⁶- | (dt, J = 9.0, 2.3 Hz, 2H), 7.55 (d, J = 9.3 Hz, 1H), 7.05 (dd, J = |
| | sulfanone | 9.3, 2.0 Hz, 1H), 6.94 (q, J = 0.9 Hz, 1H), 3.46 (s, 3H) |
| | | ESI MS (m/z): 384.15 [M + 1]. |
| 51 | ((2-(5-fluoropyridin-3- | ¹H-NMR (400 MHz, DMSO-d₆) δ 9.13 (d, J = 1.5 Hz, 1H), |
| | yl)-2H-indazol-5- | 8.88 (d, J = 0.9 Hz, 1H), 8.61 (d, J = 2.4 Hz, 1H), 8.35 (dt, J = |
| | yl)imino)(methyl)(p- | 10.1, 2.3 Hz, 1H), 7.83 (dd, J = 6.6, 1.7 Hz, 2H), 7.52 (dt, J = |
| | tolyl)-λ⁶-sulfanone | 9.2, 0.9 Hz, 1H), 7.40 (d, J = 7.9 Hz, 2H), 7.03 (dd, J = 9.2, |
| | | 2.1 Hz, 1H), 6.95 (q, J = 1.0 Hz, 1H), 3.38 (s, 3H), 2.34 (s, |
| | | 3H); ESI MS (m/z): 380.9 [M + 1]. |
| 52 | methyl((2-(pyridazin-4- | ¹H-NMR (400 MHz, DMSO-d₆) δ 9.93 (dd, J = 2.9, 1.0 Hz, |
| | yl)-2H-indazol-5- | 1H), 9.33 (dd, J = 5.9, 1.0 Hz, 1H), 9.03 (d, J = 0.7 Hz, 1H), |
| | yl)imino)(p-tolyl)-λ⁶- | 8.20 (q, J = 2.9 Hz, 1H), 7.83 (d, J = 8.3 Hz, 2H), 7.54 (d, J = |
| | sulfanone | 9.3 Hz, 1H), 7.40 (d, J = 7.8 Hz, 2H), 7.05 (dd, J = 9.3, 2.0 Hz, |
| | | 1H), 6.92 (q, J = 0.9 Hz, 1H), 3.39 (s, 3H), 2.34 (s, 3H) |
| | | ESI MS (m/z): 364.3 [M + 1]. |
| 53 | ethyl((2-(5- | ¹H-NMR (400 MHz, DMSO-d₆) δ 9.12 (s, 1H), 8.88 (d, J = |
| | fluoropyridin-3-yl)-2H- | 1.0 Hz, 1H), 8.61 (d, J = 2.2 Hz, 1H), 8.35 (dt, J = 10.2, 2.3 |
| | indazol-5- | Hz, 1H), 7.92-7.89 (m, 2H), 7.68-7.59 (m, 3H), 7.53 (d, J = |
| | yl)imino)(phenyl)-λ⁶- | 9.3 Hz, 1H), 7.06 (dd, J = 9.2, 2.1 Hz, 1H), 6.97 (q, J = 0.9 Hz, |
| | sulfanone | 1H), 3.56-3.47 (m, 2H), 1.19-1.15 (m, 3H); ESI MS (m/z): |
| | | 380.9 [M + 1]. |
| 54 | ethyl(methyl)((2- | ¹H-NMR (400 MHz, DMSO-d₆) δ 10.00 (q, J = 1.2 Hz, 1H), |
| | (pyridazin-4-yl)-2H- | 9.36 (dd, J = 5.9, 1.0 Hz, 1H), 9.11 (d, J = 0.7 Hz, 1H), 8.27 |
| | indazol-5-yl)imino)-λ⁶- | (q, J = 2.9 Hz, 1H), 7.59 (d, J = 9.0 Hz, 1H), 7.14 (q, J = 1.0 |
| | sulfanone | Hz, 1H), 7.02 (dd, J = 9.3, 2.0 Hz, 1H), 3.39 (q, J = 7.4 Hz, |
| | | 2H), 3.14 (s, 3H), 1.29 (t, J = 7.5 Hz, 3H); ESI MS (m/z): |
| | | 301.9 [M + 1]. |
| 55 | (4-chlorophenyl)((2-(5- | ¹H-NMR (400 MHz, DMSO-d₆) δ 9.13 (s, 1H), 8.90 (d, J = |
| | fluoropyridin-3-yl)-2H- | 0.7 Hz, 1H), 8.61 (d, J = 2.4 Hz, 1H), 8.36 (dt, J = 10.0, 2.3 |
| | indazol-5- | Hz, 1H), 7.96 (dt, J = 9.0, 2.3 Hz, 2H), 7.69 (dt, J = 9.1, 2.2 |
| | yl)imino)(methyl)-λ⁶- | Hz, 2H), 7.54 (d, J = 9.0 Hz, 1H), 7.02 (dd, J = 9.2, 2.0 Hz, |
| | sulfanone | 1H), 6.96 (q, J = 0.9 Hz, 1H), 3.45 (s, 3H); ESI MS (m/z): |
| | | 400.9 [M + 1]. |
| 56 | 1-((2-(pyridazin-4-yl)- | ¹H-NMR (400 MHz, CHLOROFORM-D) δ 9.85 (d, J = 2.2 |
| | 2H-indazol-5- | Hz, 1H), 9.30 (t, J = 5.9 Hz, 1H), 8.38 (s, 1H), 8.03-8.00 (m, |
| | yl)imino)tetrahydro- | 1H), 7.63 (d, J = 9.0 Hz, 1H), 7.24 (s, 1H), 7.14 (dd, J = 9.3, |
| | 1H-λ⁶-thiophene 1- | 2.0 Hz, 1H), 3.53-3.46 (m, 2H), 3.26-3.19 (m, 2H), 2.39-2.29 |
| | oxide | (m, 5H), 1.33-1.25 (m, 3H); ESI MS (m/z): 313.9 [M + 1]. |
| 57 | (3- | ¹H-NMR (400 MHz, CHLOROFORM-D) δ 9.78 (q, J = 1.2 |
| | fluorophenyl)(methyl)((2- | Hz, 1H), 9.26 (dd, J = 5.9, 1.0 Hz, 1H), 8.29 (d, J = 1.0 Hz, |
| | (pyridazin-4-yl)-2H- | 1H), 7.93 (q, J = 2.9 Hz, 1H), 7.78 (dq, J = 7.8, 0.9 Hz, 1H), |
| | indazol-5-yl)imino)-λ⁶- | 7.72-7.69 (m, 1H), 7.59-7.50 (m, 2H), 7.29 (tdd, J = 8.3, 2.5, |
| | sulfanone | 0.9 Hz, 1H), 7.16 (dd, J = 9.3, 2.0 Hz, 1H), 7.12 (q, J = 0.9 Hz, |
| | | 1H), 3.30 (s, 3H); ESI MS (m/z): 368 [M + 1]. |
| 58 | (3-fluorophenyl)((2-(5- | ¹H-NMR (400 MHz, CHLOROFORM-D) δ 8.92-8.19 (m, |
| | fluoropyridin-3-yl)-2H- | 2H), 8.91 (d, J = 1.5 Hz, 1H), 8.46 (d, J = 2.4 Hz, 1H), 8.20 (d, |
| | indazol-5- | J = 1.0 Hz, 1H), 8.02 (dt, J = 9.4, 2.4 Hz, 1H), 7.79 (dq, J = |
| | yl)imino)(methyl)-λ⁶- | 7.8, 0.9 Hz, 1H), 7.72 (dt, J = 7.8, 2.1 Hz, 1H), 7.59 (dt, J = |
| | sulfanone | 9.0, 1.0 Hz, 1H), 7.52 (td, J = 8.1, 5.1 Hz, 1H), 7.31-7.27 (m, |
| | | 1H), 7.17-7.14 (m, 2H), 3.30 (s, 3H); ESI MS (m/z): 384.9 |
| | | [M + 1]. |
| 59 | phenyl((2-(pyridin-3- | ¹H-NMR (400 MHz, CHLOROFORM-D) δ 9.09 (d, J = 2.2 |
| | yl)-2H-indazol-5- | Hz, 1H), 8.60 (dd, J = 4.6, 1.5 Hz, 1H), 8.21-8.18 (m, 2H), |
| | yl)imino)(3,3,3- | 7.98-7.96 (m, 2H), 7.66-7.55 (m, 4H), 7.46-7.42 (m, 1H), |
| | trifluoropropyl)-λ⁶- | 7.18-7.14 (m, 2H), 3.61-3.55 (m, 1H), 3.49-3.41 (m, 1H), |
| | sulfanone | 2.78-2.64 (m, 2H); ESI MS (m/z): 431 [M + 1]. |
| 60 | (4-methoxyphenyl)((2- | ¹H-NMR (400 MHz, CHLOROFORM-D) δ 9.09 (d, J = 2.2 |
| | (pyridin-3-yl)-2H- | Hz, 1H), 8.60 (dd, J = 4.6, 1.5 Hz, 1H), 8.19 (ddd, J = 8.6, 2.9, |
| | indazol-5- | 1.3 Hz, 2H), 8.00 (dd, J = 7.8, 1.7 Hz, 1H), 7.57-7.52 (m, 2H), |
| | yl)imino)(3,3,3- | 7.43 (ddd, J = 8.3, 4.9, 0.7 Hz, 1H), 7.15 (q, J = 0.9 Hz, 1H), |
| | trifluoropropyl)-λ⁶- | 7.09-7.05 (m, 2H), 7.01-6.99 (m, 1H), 3.96 (s, 3H), 3.89-3.72 |
| | sulfanone | (m, 2H), 2.86-2.75 (m, 1H), 2.62-2.51 (m, 1H) |
| | | ESI MS (m/z): 461 [M + 1]. |
| 61 | (4-fluorophenyl)((2- | ¹H-NMR (400 MHz, DMSO-d₆) δ 9.21 (d, J = 2.4 Hz, 1H), |
| | (pyridin-3-yl)-2H- | 8.88 (d, J = 0.7 Hz, 1H), 8.59 (dd, J = 4.8, 1.3 Hz, 1H), 8.36 |
| | indazol-5- | (dq, J = 8.3, 1.4 Hz, 1H), 8.06-8.02 (m, 2H), 7.60-7.55 (m, |
| | yl)imino)(3,3,3- | 2H), 7.50-7.44 (m, 2H), 7.08-7.04 (m, 2H), 3.90-3.76 (m, 2H), |
| | trifluoropropyl)-λ⁶- | 2.82-2.72 (m, 2H); ESI MS (m/z): 449 [M + 1]. |
| | sulfanone | |
| 62 | phenyl((2-(pyrimidin-5- | ¹H-NMR (400 MHz, CHLOROFORM-D) δ 9.25 (s, 2H), 9.19 |
| | yl)-2H-indazol-5- | (s, 1H), 8.18 (d, J = 1.0 Hz, 1H), 7.98-7.95 (m, 2H), 7.67-7.55 |
| | yl)imino)(3,3,3- | (m, 4H), 7.19-7.15 (m, 2H), 3.63-3.41 (m, 2H), 2.83-2.61 (m, |
| | trifluoropropyl)-λ⁶- | 2H); ESI MS (m/z): 432 [M + 1]. |
| | sulfanone | |
| 63 | (4-methoxyphenyl)((2- | ¹H-NMR (400 MHz, CHLOROFORM-D) δ 9.25 (s, 2H), 9.19 |
| | (pyrimidin-5-yl)-2H- | (s, 1H), 8.17 (d, J = 1.0 Hz, 1H), 8.00 (dd, J = 8.1, 1.7 Hz, |
| | indazol-5- | 1H), 7.57-7.52 (m, 2H), 7.13-7.06 (m, 3H), 7.01-6.99 (m, 1H), |
| | yl)imino)(3,3,3- | 3.96 (s, 3H), 3.89-3.71 (m, 2H), 2.82-2.77 (m, 1H), 2.61-2.55 |
| | trifluoropropyl)-λ⁶- | (m, 1H); ESI MS (m/z): 462.2 [M + 1]. |
| | sulfanone | |
| 64 | (4-fluorophenyl)((2- | ¹H-NMR (400 MHz, DMSO-d₆) δ 9.42 (s, 2H), 9.20 (s, 1H), |
| | (pyrimidin-5-yl)-2H- | 8.94 (d, J = 1.0 Hz, 1H), 8.04 (dd, J = 8.9, 5.3 Hz, 2H), 7.58 |
| | indazol-5- | (d, J = 9.0 Hz, 1H), 7.47 (t, J = 8.9 Hz, 2H), 7.09 (dd, J = 9.0, |
| | yl)imino)(3,3,3- | 2.0 Hz, 1H), 7.04 (d, J = 1.2 Hz, 1H), 3.88-3.81 (m, 2H), 2.81- |
| | trifluoropropyl)-λ⁶- | 2.73 (m, 2H); ESI MS (m/z): 450 [M + 1]. |
| | sulfanone | |
| 65 | ((2-(5-fluoropyridin-3- | ¹H-NMR (400 MHz, CHLOROFORM-D) δ 8.91 (d, J = 1.7 |
| | yl)-2H-indazol-5- | Hz, 1H), 8.46 (d, J = 2.2 Hz, 1H), 8.18 (d, J = 1.0 Hz, 1H), |
| | yl)imino)(phenyl)(3,3,3- | 8.03-7.95 (m, 3H), 7.66-7.55 (m, 4H), 7.16 (dd, J = 8.8, 2.0 |
| | trifluoropropyl)-λ⁶- | Hz, 2H), 3.62-3.41 (m, 2H), 2.82-2.60 (m, 2H); ESI MS (m/z): |
| | sulfanone | 449 [M + 1]. |
| 66 | ((2-(5-fluoropyridin-3- | ¹H-NMR (400 MHz, CHLOROFORM-D) δ 8.91 (d, J = 1.5 |
| | yl)-2H-indazol-5- | Hz, 1H), 8.46 (d, J = 2.4 Hz, 1H), 8.18 (d, J = 0.7 Hz, 1H), |
| | yl)imino)(4- | 8.03-7.99 (m, 2H), 7.57-7.52 (m, 2H), 7.12 (q, J = 0.9 Hz, |
| | methoxyphenyl)(3,3,3- | 1H), 7.09-7.05 (m, 2H), 7.00 (d, J = 8.3 Hz, 1H), 3.95 (d, J = |
| | trifluoropropyl)-λ⁶- | 10.5 Hz, 3H), 3.89-3.71 (m, 2H), 2.82-2.52 (m, 2H); ESI MS |
| | sulfanone | (m/z): 478.9 [M + 1]. |
| 67 | (4-fluorophenyl)((2-(5- | ¹H-NMR (400 MHz, CHLOROFORM-D) δ 8.91 (t, J = 0.8 |
| | fluoropyridin-3-yl)-2H- | Hz, 1H), 8.47 (d, J = 2.2 Hz, 1H), 8.19 (d, J = 1.0 Hz, 1H), |
| | indazol-5- | 8.04-7.95 (m, 3H), 7.61-7.59 (m, 1H), 7.26-7.21 (m, 2H), |
| | yl)imino)(3,3,3- | 7.15-7.12 (m, 2H), 3.63-3.55 (m, 1H), 3.48-3.40 (m, 1H), |
| | trifluoropropyl)-λ⁶- | 2.83-2.61 (m, 2H) |
| | sulfanone | ESI MS (m/z): 466.9 [M + 1]. |
| 68 | phenyl((2-(pyridazin-4- | ¹H-NMR (400 MHz, CHLOROFORM-D) δ 9.77 (dd, J = 2.9, |
| | yl)-2H-indazol-5- | 1.0 Hz, 1H), 9.26 (dd, J = 5.9, 1.0 Hz, 1H), 8.27 (d, J = 0.7 Hz, |
| | yl)imino)(3,3,3- | 1H), 7.98-7.92 (m, 3H), 7.67-7.63 (m, 1H), 7.60-7.56 (m, 3H), |
| | trifluoropropyl)-λ⁶- | 7.17 (dd, J = 9.3, 2.2 Hz, 1H), 7.10 (q, J = 0.9 Hz, 1H), 3.63- |
| | sulfanone | 3.41 (m, 2H), 2.83-2.60 (m, 2H); ESI MS (m/z): 432.2 [M + 1]. |
| 69 | (4-methoxyphenyl)((2- | ¹H-NMR (400 MHz, CHLOROFORM-D) δ 9.78 (q, J = 1.3 |
| | (pyridazin-4-yl)-2H- | Hz, 1H), 9.26 (dd, J = 5.6, 1.0 Hz, 1H), 8.29 (d, J = 0.7 Hz, |
| | indazol-5- | 1H), 8.00-7.93 (m, 3H), 7.59 (dt, J = 9.2, 0.9 Hz, 1H), 7.25- |
| | yl)imino)(3,3,3- | 7.22 (m, 2H), 7.15 (dd, J = 9.3, 2.0 Hz, 1H), 7.09 (q, J = 1.0 |
| | trifluoropropyl)-λ⁶- | Hz, 1H), 3.63-3.40 (m, 2H), 2.84-2.61 (m, 2H); ESI MS (m/z): |
| | sulfanone | 462.3 [M + 1]. |
| 70 | (4-fluorophenyl)((2- | ¹H-NMR (400 MHz, CHLOROFORM-D) δ 9.78 (q, J = 1.3 |
| | (pyridazin-4-yl)-2H- | Hz, 1H), 9.26 (dd, J = 5.6, 1.0 Hz, 1H), 8.29 (d, J = 0.7 Hz, |
| | indazol-5- | 1H), 8.00-7.93 (m, 3H), 7.59 (dt, J = 9.2, 0.9 Hz, 1H), 7.25- |
| | yl)imino)(3,3,3- | 7.22 (m, 2H), 7.15 (dd, J = 9.3, 2.0 Hz, 1H), 7.09 (q, J = 1.0 |
| | trifluoropropyl)-λ⁶- | Hz, 1H), 3.63-3.40 (m, 2H), 2.84-2.61 (m, 2H); ESI MS(m/z): |
| | sulfanone | 450.3 [M + 1]. |
| 71 | ethyl(isopropyl)((2- | ¹H-NMR (400 MHz, DMSO-d₆) δ 9.34 (d, J = 2.4 Hz, 1H), |
| | (pyridin-3-yl)-2H- | 9.02 (s, 1H), 8.61 (dd, J = 4.8, 1.3 Hz, 1H), 8.51-8.48 (m, 1H), |
| | indazol-4-yl)imino)-λ⁶- | 7.61-7.58 (m, 1H), 7.17-7.08 (m, 2H), 6.61 (d, J = 6.4 Hz, |
| | sulfanone | 1H), 3.64-3.57 (m, 1H), 3.40-3.34 (m, 2H), 1.44 (d, J = 6.6 |
| | | Hz, 3H), 1.36 (d, J = 6.8 Hz, 3H), 1.27 (t, J = 7.3 Hz, 3H) |
| | | ESI MS (m/z): 329.3 [M + 1]. |
| 72 | dimethyl((2-(pyridin-3- | ¹H-NMR (400 MHz, DMSO-d₆) δ 9.32 (d, J = 2.4 Hz, 1H), |
| | yl)-2H-indazol-4- | 8.99 (d, J = 0.7 Hz, 1H), 8.60 (dd, J = 4.8, 1.3 Hz, 1H), 8.50- |
| | yl)imino)-λ⁶-sulfanone | 8.47 (m, 1H), 7.61-7.58 (m, 1H), 7.20-7.12 (m, 2H), 6.58 (dd, |
| | | J = 6.8, 0.7 Hz, 1H), 3.32 (s, 6H); ESI MS (m/z): 287.1 |
| | | [M + 1]. |
| 73 | diethyl((2-(pyridin-3- | ¹H-NMR (400 MHz, DMSO-d₆) δ 9.33 (d, J = 2.7 Hz, 1H), |
| | yl)-2H-indazol-4- | 9.01 (s, 1H), 8.60 (dd, J = 4.8, 1.3 Hz, 1H), 8.51-8.48 (m, 1H), |
| | yl)imino)-λ⁶-sulfanone | 7.61-7.58 (m, 1H), 7.18-7.10 (m, 2H), 6.59 (d, J = 6.8 Hz, |
| | | 1H), 3.39 (q, J = 7.4 Hz, 4H), 1.30 (t, J = 7.3 Hz, 6H) |
| | | ESI MS (m/z): 315.15 [M + 1]. |
| 74 | isopropyl(methyl)((2- | ¹H-NMR (400 MHz, CHLOROFORM-D) δ 9.19 (d, J = 2.2 |
| | (pyridin-3-yl)-2H- | Hz, 1H), 8.63 (dd, J = 4.9, 1.5 Hz, 1H), 8.59 (d, J = 1.0 Hz, |
| | indazol-4-yl)imino)-λ⁶- | 1H), 8.30-8.27 (m, 1H), 7.48-7.45 (m, 1H), 7.37 (d, J = 8.8 |
| | sulfanone | Hz, 1H), 7.21 (dd, J = 8.8, 7.1 Hz, 1H), 6.79-6.77 (m, 1H), |
| | | 3.55-3.48 (m, 1H), 3.02 (s, 3H), 1.58 (d, J = 6.8 Hz, 3H), 1.54 |
| | | (d, J = 6.8 Hz, 3H); ESI MS (m/z): 315.3 [M + 1]. |
| 75 | ((2-(5-fluoropyridin-3- | ¹H-NMR (400 MHz, CHLOROFORM-D) δ 9.01 (d, J = 1.5 |
| | yl)-2H-indazol-4- | Hz, 1H), 8.56 (d, J = 1.0 Hz, 1H), 8.48 (d, J = 2.4 Hz, 1H), |
| | yl)imino)diisobutyl-λ⁶- | 8.10 (dt, J = 9.3, 2.3 Hz, 1H), 7.32-7.29 (m, 1H), 7.20 (dd, J = |
| | sulfanone | 8.8, 7.1 Hz, 1H), 6.74 (dd, J = 7.3, 0.5 Hz, 1H), 3.25 (dd, J = |
| | | 14.2, 6.1 Hz, 2H), 3.08 (td, J = 13.8, 6.6 Hz, 2H), 2.49-2.39 |
| | | (m, 2H), 1.16-1.11 (m, 12H); ESI MS (m/z): 389.4 [M + 1]. |
| 76 | dipropyl((2-(pyrimidin- | ¹H-NMR (400 MHz, CHLOROFORM-D) δ 9.35 (s, 2H), 9.22 |
| | 5-yl)-2H-indazol-4- | (s, 1H), 8.59 (d, J = 1.0 Hz, 1H), 7.35 (d, J = 8.8 Hz, 1H), 7.21 |
| | yl)imino)-λ⁶-sulfanone | (dd, J = 8.8, 7.1 Hz, 1H), 6.76 (dd, J = 7.1, 0.5 Hz, 1H), 3.30- |
| | | 3.16 (m, 4H), 1.97-1.88 (m, 4H), 1.07 (q, J = 7.3 Hz, 6H) |
| | | ESI MS (m/z): 344.3 [M + 1]. |
| 77 | ((2-(5-fluoropyridin-3- | ¹H-NMR (400 MHz, CHLOROFORM-D) δ 9.02 (d, J = 1.7 |
| | yl)-2H-indazol-4- | Hz, 1H), 8.59 (d, J = 1.0 Hz, 1H), 8.49 (d, J = 2.7 Hz, 1H), |
| | yl)imino)dipropyl-λ⁶- | 8.11 (dt, J = 9.3, 2.3 Hz, 1H), 7.34 (d, J = 8.6 Hz, 1H), 7.20 |
| | sulfanone | (dd, J = 8.8, 7.1 Hz, 1H), 6.76-6.74 (m, 1H), 3.30-3.16 (m, |
| | | 4H), 2.96 (s, 1H), 2.88 (d, J = 0.5 Hz, 1H), 1.98-1.88 (m, 4H), |
| | | 1.08 (t, J = 7.5 Hz, 6H); ESI MS (m/z): 361 [M + 1]. |
| 78 | dipropyl((2-(pyridin-3- | ¹H-NMR (400 MHz, DMSO-d₆) δ 9.33 (d, J = 2.7 Hz, 1H), |
| | yl)-2H-indazol-4- | 8.98 (s, 1H), 8.61 (dd, J = 4.6, 1.5 Hz, 1H), 8.50-8.47 (m, 1H), |
| | yl)imino)-λ⁶-sulfanone | 7.60 (dd, J = 8.1, 4.6 Hz, 1H), 7.17-7.09 (m, 2H), 6.58 (d, J = |
| | | 6.1 Hz, 1H), 3.42-3.33 (m, 4H), 1.81 (dt, J = 23.6, 7.4 Hz, |
| | | 4H), 0.98 (t, J = 7.5 Hz, 6H); ESI MS (m/z): 343.3 [M + 1]. |
| 79 | diisobutyl((2-(pyridin- | ¹H-NMR (400 MHz, DMSO-d₆) δ 9.27 (d, J = 2.4 Hz, 1H), |
| | 3-yl)-2H-indazol-5- | 8.92 (s, 1H), 8.60 (d, J = 3.4 Hz, 1H), 8.41 (d, J = 9.5 Hz, 1H), |
| | yl)imino)-λ⁶-sulfanone | 7.62-7.55 (m, 4H), 7.16 (s, 1H), 6.98 (dd, J = 9.0, 2.0 Hz, 1H), |
| | | 3.24-3.17 (m, 4H), 2.29 (t, J = 6.6 Hz, 2H), 1.09-1.01 (m, |
| | | 12H); ESI MS (m/z): 371.1 [M + 1]. |
| 80 | ((2-(5-fluoropyridin-3- | ¹H-NMR (400 MHz, DMSO-d₆) δ 9.19 (s, 1H), 8.97 (s, 1H), |
| | yl)-2H-indazol-5- | 8.63 (d, J = 2.7 Hz, 1H), 8.41 (dt, J = 10.1, 2.3 Hz, 1H), 7.56 |
| | yl)imino)diisobutyl-λ⁶- | (d, J = 9.3 Hz, 1H), 7.14 (d, J = 1.2 Hz, 1H), 7.00 (dd, J = 9.0, |
| | sulfanone | 2.0 Hz, 1H), 3.27-3.18 (m, 4H), 2.32-2.24 (m, 2H), 1.09-1.01 |
| | | (m, 12H); ESI MS (m/z): 389.2 [M + 1]. |
| 81 | 1-((2-(pyrimidin-5-yl)- | ¹H-NMR (400 MHz, DMSO-d₆) δ 9.54 (s, 2H), 9.21 (s, 1H), |
| | 2H-indazol-4- | 9.13 (s, 1H), 7.22-7.16 (m, 2H), 6.52 (dd, J = 6.4, 1.2 Hz, 1H), |
| | yl)imino)tetrahydro- | 3.48-3.33 (m, 4H), 2.32-2.10 (m, 4H); ESI MS (m/z): 314 |
| | 1H-λ⁶-thiophene 1- | [M + 1]. |
| | oxide | |
| 82 | dimethyl((2-(pyrimidin- | ¹H-NMR (400 MHz, DMSO-d₆) δ 9.53 (s, 2H), 9.21 (s, 1H), |
| | 5-yl)-2H-indazol-4- | 9.09 (d, J = 0.7 Hz, 1H), 7.19-7.15 (m, 2H), 6.60 (dd, J = 6.5, |
| | yl)imino)-λ⁶-sulfanone | 1.3 Hz, 1H), 3.33 (s, 6H); ESI MS (m/z): 287.9 [M + 1]. |
| 83 | diethyl((2-(pyrimidin-5- | ¹H-NMR (400 MHz, DMSO-d₆) δ 9.54 (s, 2H), 9.21 (s, 1H), |
| | yl)-2H-indazol-4- | 9.11 (d, J = 0.7 Hz, 1H), 7.19-7.12 (m, 2H), 6.61 (dd, J = 6.6, |
| | yl)imino)-λ⁶-sulfanone | 1.0 Hz, 1H), 3.40 (q, J = 7.4 Hz, 4H), 1.31 (t, J = 7.3 Hz, 6H) |
| | | ESI MS (m/z): 316.1 [M + 1]. |
| 84 | isopropyl(methyl)((2- | ¹H-NMR (400 MHz, DMSO-d₆) δ 9.54 (d, J = 3.7 Hz, 2H), |
| | (pyrimidin-5-yl)-2H- | 9.21 (s, 1H), 9.11 (d, J = 0.5 Hz, 1H), 7.19-7.13 (m, 2H), 6.62 |
| | indazol-4-yl)imino)-λ⁶- | (dd, J = 6.1, 1.5 Hz, 1H), 3.63-3.56 (m, 1H), 3.15 (s, 3H), 1.48 |
| | sulfanone | (d, J = 6.6 Hz, 3H), 1.42 (d, J = 6.8 Hz, 3H); ESI MS (m/z): |
| | | 315.9 [M + 1]. |
| 85 | ethyl(isopropyl)((2- | ¹H-NMR (400 MHz, DMSO-d₆) δ 9.55 (s, 2H), 9.21 (s, 1H), |
| | (pyrimidin-5-yl)-2H- | 9.12 (d, J = 0.7 Hz, 1H), 7.18-7.11 (m, 2H), 6.62 (dd, J = 6.5, |
| | indazol-4-yl)imino)-λ⁶- | 1.3 Hz, 1H), 3.65-3.58 (m, 1H), 3.41-3.34 (m, 2H), 1.45 (d, J = |
| | sulfanone | 6.8 Hz, 3H), 1.36 (d, J = 6.8 Hz, 3H), 1.28 (t, J = 7.5 Hz, |
| | | 3H); ESI MS (m/z): 330[M + 1]. |
| 86 | 1-((2-(5-fluoropyridin- | ¹H-NMR (400 MHz, DMSO-d₆) δ 9.26 (d, J = 1.2 Hz, 1H), |
| | 3-yl)-2H-indazol-4- | 9.10 (d, J = 0.5 Hz, 1H), 8.63-8.63 (m, 1H), 8.53 (dt, J = 10.0, |
| | yl)imino)tetrahydro- | 2.3 Hz, 1H), 7.20-7.15 (m, 2H), 6.51 (dd, J = 6.0, 1.8 Hz, 1H), |
| | 1H-λ⁶-thiophene 1- | 3.47-3.34 (m, 4H), 2.31-2.10 (m, 4H); ESI MS (m/z): |
| | oxide | 331[M + 1]. |
| 87 | ((2-(5-fluoropyridin-3- | ¹H-NMR (400 MHz, DMSO-d₆) δ 9.25 (t, J = 1.3 Hz, 1H), |
| | yl)-2H-indazol-4- | 9.06 (d, J = 0.7 Hz, 1H), 8.63-8.63 (m, 1H), 8.52 (dt, J = 10.0, |
| | yl)imino)dimethyl-λ⁶- | 2.3 Hz, 1H), 7.20-7.14 (m, 2H), 6.59 (dd, J = 6.2, 1.6 Hz, 1H), |
| | sulfanone | 3.32 (s, 6H); ESI MS (m/z): 305[M + 1]. |
| 88 | 1-((2-(pyridin-3-yl)-2H- | ¹H-NMR (400 MHz, DMSO-d₆) δ 9.33 (d, J = 2.2 Hz, 1H), |
| | indazol-4- | 9.03 (d, J = 0.7 Hz, 1H), 8.60 (dd, J = 4.6, 1.5 Hz, 1H), 8.49 |
| | yl)imino)tetrahydro- | (dq, J = 8.3, 1.4 Hz, 1H), 7.60 (ddd, J = 8.4, 4.8, 0.7 Hz, 1H), |
| | 1H-λ⁶-thiophene 1- | 7.21-7.13 (m, 2H), 6.51 (dd, J = 6.7, 0.9 Hz, 1H), 3.47-3.41 |
| | oxide | (m, 2H), 3.36-3.33 (m, 2H), 2.29-2.21 (m, 2H), 2.20-2.14 (m, |
| | | 2H), 1.90 (s, 1H), 1.22 (s, 1H); ESI MS (m/z): 389.2 [M + 1]. |
| 89 | diethyl((2-(5- | ¹H-NMR (400 MHz, DMSO-d₆) δ 9.27 (d, J = 1.5 Hz, 1H), |
| | fluoropyridin-3-yl)-2H- | 9.08 (d, J = 1.0 Hz, 1H), 8.63-8.63 (m, 1H), 8.53 (dt, J = 10.0, |
| | indazol-4-yl)imino)-λ⁶- | 2.3 Hz, 1H), 7.17-7.11 (m, 2H), 6.59 (dd, J = 6.5, 1.3 Hz, 1H), |
| | sulfanone | 3.39 (q, J = 7.4 Hz, 4H), 1.31 (t, J = 7.3 Hz, 6H); ESI MS |
| | | (m/z): 333 [M + 1]. |
| 90 | ((2-(5-fluoropyridin-3- | ¹H-NMR (400 MHz, DMSO-d₆) δ 9.27 (t, J = 1.3 Hz, 1H), |
| | yl)-2H-indazol-4- | 9.08 (s, 1H), 8.64-8.63 (m, 1H), 8.54 (dt, J = 10.1, 2.4 Hz, |
| | yl)imino)(isopropyl)(methyl)- | 1H), 7.17-7.12 (m, 2H), 6.61 (dd, J = 5.7, 2.1 Hz, 1H), 3.62- |
| | λ⁶-sulfanone | 3.55 (m, 1H), 3.14 (s, 3H), 1.48 (d, J = 6.8 Hz, 3H), 1.42 (d, |
| | | J = 6.8 Hz, 3H); ESI MS (m/z): 333 [M + 1]. |
| 91 | ethyl((2-(5- | ¹H-NMR (400 MHz, DMSO-d₆) δ 9.27 (q, J = 1.0 Hz, 1H), |
| | fluoropyridin-3-yl)-2H- | 9.09 (d, J = 0.7 Hz, 1H), 8.63-8.63 (m, 1H), 8.54 (dt, J = 10.1, |
| | indazol-4- | 2.4 Hz, 1H), 7.16-7.10 (m, 2H), 6.61 (dd, J = 6.2, 1.6 Hz, 1H), |
| | yl)imino)(isopropyl)-λ⁶- | 3.64-3.58 (m, 1H), 3.41-3.35 (m, 2H), 1.45 (d, J = 6.8 Hz, |
| | sulfanone | 3H), 1.36 (d, J = 6.8 Hz, 3H), 1.28 (t, J = 7.5 Hz, 3H); ESI MS |
| | | (m/z): 347.2 [M + 1]. |
| 99 | isopropyl((2-(pyridin-3- | ¹H-NMR (400 MHz, DMSO-d₆) δ 9.27 (d, J = 2.4 Hz, 1H), |
| | yl)-2H-indazol-5- | 8.94 (d, J = 0.6 Hz, 1H), 8.60 (dd, J = 4.6, 1.5 Hz, 1H), 8.41 |
| | yl)imino)(3,3,3- | (dq, J = 8.3, 1.4 Hz, 1H), 7.62-7.58 (m, 2H), 7.21 (q, J = 0.9 |
| | trifluoropropyl)-λ⁶- | Hz, 1H), 7.04 (dd, J = 9.2, 2.1 Hz, 1H), 3.70-3.63 (m, 1H), |
| | sulfanone | 3.52-3.48 (m, 2H), 2.85-2.73 (m, 2H), 1.40 (d, J = 6.7 Hz, |
| | | 3H), 1.34 (d, J = 6.7 Hz, 3H); ESI MS (m/z): 397 [M + 1]. |
| 100 | ((2-(5-fluoropyridin-3- | ¹H-NMR (400 MHz, DMSO-d₆) δ 9.20 (s, 1H), 8.99 (d, J = |
| | yl)-2H-indazol-5- | 0.9 Hz, 1H), 8.64 (d, J = 2.8 Hz, 1H), 8.43 (dt, J = 10.1, 2.4 |
| | yl)imino)(isopropyl)(3,3,3- | Hz, 1H), 7.59 (d, J = 9.5 Hz, 1H), 7.20 (q, J = 0.9 Hz, 1H), |
| | trifluoropropyl)-λ⁶- | 7.06 (dd, J = 9.2, 1.8 Hz, 1H), 3.71-3.61 (m, 1H), 3.53-3.49 |
| | sulfanone | (m, 2H), 2.85-2.72 (m, 2H), 1.40 (d, J = 6.7 Hz, 3H), 1.34 (d, |
| | | J = 7.0 Hz, 3H); ESI MS (m/z): 415 [M + 1]. |
| 101 | isopropyl((2- | ¹H-NMR (400 MHz, DMSO-d₆) δ 9.47 (s, 2H), 9.22 (s, 1H), |
| | (pyrimidin-5-yl)-2H- | 9.00 (d, J = 0.9 Hz, 1H), 7.61 (dd, J = 9.2, 0.9 Hz, 1H), 7.22 |
| | indazol-5- | (q, J = 0.9 Hz, 1H), 7.06 (dd, J = 9.2, 2.1 Hz, 1H), 3.71-3.64 |
| | yl)imino)(3,3,3- | (m, 1H), 3.53-3.49 (m, 2H), 2.85-2.73 (m, 2H), 1.40 (d, J = |
| | trifluoropropyl)-λ⁶- | 6.7 Hz, 3H), 1.34 (d, J = 6.7 Hz, 3H); ESI MS (m/z): 398 |
| | sulfanone | [M + 1]. |
| 102 | isopropyl((2-(pyridin-3- | ¹H-NMR (400 MHz, DMSO-d₆) δ 9.34 (d, J = 2.1 Hz, 1H), |
| | yl)-2H-indazol-4- | 9.07 (d, J = 0.9 Hz, 1H), 8.62 (dd, J = 4.7, 1.4 Hz, 1H), 8.49 |
| | yl)imino)(3,3,3- | (dq, J = 8.3, 1.4 Hz, 1H), 7.61 (ddd, J = 8.3, 4.7, 0.7 Hz, 1H), |
| | trifluoropropyl)-λ⁶- | 7.21 (d, J = 8.6 Hz, 1H), 7.13 (dd, J = 8.6, 7.0 Hz, 1H), 6.63- |
| | sulfanone | 6.61 (m, 1H), 3.78-3.71 (m, 1H), 3.58 (q, J = 5.2 Hz, 2H), |
| | | 2.92-2.79 (m, 2H), 1.47 (d, J = 6.7 Hz, 3H), 1.39 (d, J = 6.7 |
| | | Hz, 3H); ESI MS (m/z): 397 [M + 1]. |
| 103 | isopropyl((2- | ¹H-NMR (400 MHz, DMSO-d₆) δ 9.55 (d, J = 3.1 Hz, 2H), |
| | (pyrimidin-5-yl)-2H- | 9.23 (s, 1H), 9.16 (d, J = 0.9 Hz, 1H), 7.22 (dd, J = 7.9, 0.9 |
| | indazol-4- | Hz, 1H), 7.15 (dd, J = 8.7, 6.9 Hz, 1H), 6.64 (dd, J = 7.0, 0.6 |
| | yl)imino)(3,3,3- | Hz, 1H), 3.79-3.72 (m, 1H), 3.61-3.57 (m, 2H), 2.91-2.79 (m, |
| | trifluoropropyl)-λ⁶- | 2H), 1.48 (d, J = 6.7 Hz, 3H), 1.39 (d, J = 6.7 Hz, 3H) |
| | sulfanone | ESI MS (m/z): 398 [M + 1]. |
| 104 | ((2-(5-fluoropyridin-3- | ¹H-NMR (400 MHz, DMSO-d₆) δ 9.27 (s, 1H), 9.13 (d, J = |
| | yl)-2H-indazol-4- | 0.9 Hz, 1H), 8.65 (d, J = 2.8 Hz, 1H), 8.53 (dt, J = 10.2, 2.4 |
| | yl)imino)(isopropyl)(3,3,3- | Hz, 1H), 7.20 (d, J = 8.6 Hz, 1H), 7.14 (dd, J = 8.7, 6.9 Hz, |
| | trifluoropropyl)-λ⁶- | 1H), 6.62 (dd, J = 6.9, 0.8 Hz, 1H), 3.78-3.71 (m, 1H), 3.61- |
| | sulfanone | 3.57 (m, 2H), 2.91-2.79 (m, 2H), 1.48 (d, J = 6.7 Hz, 3H), 1.39 |
| | | (d, J = 7.0 Hz, 3H); ESI MS (m/z): 415 [M + 1]. |
| 108 | ethyl((7-fluoro-2- | ¹H-NMR (400 MHz, DMSO-d₆) δ 9.35 (d, J = 2.2 Hz, 1H), |
| | (pyridin-3-yl)-2H- | 9.12 (d, J = 2.7 Hz, 1H), 8.65 (dd, J = 4.8, 1.3 Hz, 1H), 8.52 |
| | indazol-4- | (dq, J = 8.3, 1.4 Hz, 1H), 7.62 (ddd, J = 8.3, 4.6, 0.7 Hz, 1H), |
| | yl)imino)(isopropyl)-λ⁶- | 6.91 (dd, J = 11.7, 7.8 Hz, 1H), 6.48 (dd, J = 7.9, 3.3 Hz, 1H), |
| | sulfanone | 3.62-3.55 (m, 1H), 2.97-2.87 (m, 1H), 1.43 (d, J = 6.6 Hz, |
| | | 3H), 1.35 (d, J = 6.8 Hz, 3H), 1.26 (t, J = 7.5 Hz, 3H), 1.22- |
| | | 1.17 (m, 4H); ESI MS (m/z): 347[M + 1]. |
| 109 | ethyl((7-fluoro-2- | ¹H-NMR (400 MHz, DMSO-d₆) δ 9.33 (d, J = 2.2 Hz, 1H), |
| | (pyridin-3-yl)-2H- | 9.11 (d, J = 2.7 Hz, 1H), 8.64 (dd, J = 4.6, 1.5 Hz, 1H), 8.51 |
| | indazol-4- | (dq, J = 8.3, 1.4 Hz, 1H), 7.62 (ddd, J = 8.4, 4.8, 0.7 Hz, 1H), |
| | yl)imino)(methyl)-λ⁶- | 6.93 (dd, J = 11.9, 7.9 Hz, 1H), 6.46 (dd, J = 7.9, 3.3 Hz, 1H), |
| | sulfanone | 3.42 (q, J = 7.3 Hz, 2H), 3.19 (s, 3H), 1.35 (t, J = 7.3 Hz, 3H) |
| | | ESI MS (m/z): 319 [M + 1]. |
| 136 | ethyl(propyl)((2- | ¹H-NMR (400 MHz, DMSO-d₆) δ 9.33 (d, J = 2.2 Hz, 1H), |
| | (pyridin-3-yl)-2H- | 8.99 (d, J = 0.7 Hz, 1H), 8.61 (dd, J = 4.6, 1.5 Hz, 1H), 8.49 |
| | indazol-4-yl)imino)-λ⁶- | (dq, J = 8.3, 1.4 Hz, 1H), 7.60 (ddd, J = 8.3, 4.6, 0.7 Hz, 1H), |
| | sulfanone | 7.17 (d, J = 8.6 Hz, 1H), 7.11 (dd, J = 8.7, 7.0 Hz, 1H), 6.59 |
| | | (dd, J = 6.8, 1.0 Hz, 1H), 3.42-3.33 (m, 4H), 1.81 (dt, J = 23.6, |
| | | 7.4 Hz, 2H), 1.34-1.29 (m, 4H), 1.26-1.22 (m, 3H), 0.99 (t, J = |
| | | 7.3 Hz, 3H), 0.84 (t, J = 6.7 Hz, 1H); ESI MS(m/z): 328.55 |
| | | [M + 1]. |
| 137 | ethyl(propyl)((2- | ¹H-NMR (400 MHz, DMSO-d₆) δ 9.26 (d, J = 2.4 Hz, 1H), |
| | (pyridin-3-yl)-2H- | 8.91 (s, 1H), 8.60 (dd, J = 4.8, 1.3 Hz, 1H), 8.40 (dq, J = 8.4, |
| | indazol-5-yl)imino)-λ⁶- | 1.3 Hz, 1H), 7.62-7.55 (m, 2H), 7.17 (d, J = 1.5 Hz, 1H), 6.99 |
| | sulfanone | (dd, J = 9.0, 2.0 Hz, 1H), 3.33 (d, J = 7.3 Hz, 1H), 3.29-3.25 |
| | | (m, 3H), 1.75 (q, J = 7.8 Hz, 2H), 1.28-1.15 (m, 5H), 0.97 (t, |
| | | J = 7.5 Hz, 3H); ESI MS(m/z): 328.95 [M + 1]. |
| 138 | ethyl((2-(5- | ¹H-NMR (400 MHz, DMSO-d₆) δ 9.27 (s, 1H), 9.07 (d, J = |
| | fluoropyridin-3-yl)-2H- | 0.5 Hz, 1H), 8.63 (d, J = 2.4 Hz, 1H), 8.54 (dt, J = 10.2, 2.3 |
| | indazol-4- | Hz, 1H), 7.17-7.11 (m, 2H), 6.59 (dd, J = 6.2, 1.6 Hz, 1H), |
| | yl)imino)(propyl)-λ⁶- | 3.43-3.34 (m, 4H), 1.81 (dt, J = 23.5, 7.3 Hz, 2H), 1.31 (t, J = |
| | sulfanone | 7.5 Hz, 3H), 0.99 (t, J = 7.5 Hz, 3H); ESI MS(m/z): 346.55 |
| | | [M + 1]. |
| 139 | ((2-(5-fluoropyridin-3- | ¹H-NMR (400 MHz, DMSO-d₆) δ 9.21 (s, 1H), 9.00 (s, 1H), |
| | yl)-2H-pyrazolo[3,4- | 8.67 (d, J = 2.4 Hz, 1H), 8.46 (dt, J = 10.1, 2.4 Hz, 1H), 8.33 |
| | b]pyridin-5- | (d, J = 2.4 Hz, 1H), 7.55 (d, J = 2.4 Hz, 1H), 3.37-3.33 (m, |
| | yl)imino)dipropyl-λ⁶- | 4H), 1.77 (dt, J = 23.8, 7.3 Hz, 4H), 0.97 (t, J = 7.3 Hz, 6H) |
| | sulfanone | ESI MS (m/z): 361.55 [M + 1]. |
| 140 | (3-fluoropyridin-2- | ¹H-NMR (400 MHz, DMSO-d₆) δ 9.17 (d, J = 1.2 Hz, 1H), |
| | yl)((2-(5-fluoropyridin- | 9.00 (s, 1H), 8.66 (d, J = 2.4 Hz, 1H), 8.58 (td, J = 3.0, 1.4 Hz, |
| | 3-yl)-2H-pyrazolo[3,4- | 1H), 8.43 (dt, J = 9.9, 2.3 Hz, 1H), 8.34 (d, J = 2.7 Hz, 1H), |
| | b]pyridin-5- | 8.03-7.98 (m, 1H), 7.81-7.77 (m, 1H), 7.49 (d, J = 2.4 Hz, |
| | yl)imino)(methyl)-λ⁶- | 1H), 3.66 (s, 3H); ESI MS (m/z): 386.4 [M + 1]. |
| | sulfanone | |
| 141 | ((2-(5-fluoropyridin-3- | ¹H-NMR (400 MHz, DMSO-d₆) δ 9.15-9.13 (m, 2H), 8.94 (s, |
| | yl)-2H-pyrazolo[3,4- | 1H), 8.83 (dd, J = 4.9, 1.5 Hz, 1H), 8.66 (d, J = 2.4 Hz, 1H), |
| | b]pyridin-5- | 8.42-8.36 (m, 3H), 7.66 (ddd, J = 8.2, 4.8, 0.7 Hz, 1H), 7.40 |
| | yl)imino)(methyl)(pyridin- | (d, J = 2.7 Hz, 1H), 3.61 (s, 3H); ESI MS(m/z): 368.45 [M + 1]. |
| | 3-yl)-λ⁶-sulfanone | |
| 142 | (2-chloropyridin-3- | ¹H-NMR (400 MHz, DMSO-d₆) δ 9.14 (s, 1H), 8.94 (s, 1H), |
| | yl)((2-(5-fluoropyridin- | 8.65 (td, J = 4.7, 2.0 Hz, 2H), 8.60 (dd, J = 7.8, 2.0 Hz, 1H), |
| | 3-yl)-2H-pyrazolo[3,4- | 8.42-8.39 (m, 2H), 7.71 (dd, J = 7.8, 4.6 Hz, 1H), 7.38 (d, J = |
| | b]pyridin-5- | 2.4 Hz, 1H), 3.71 (s, 3H); ESI MS(m/z): 402.4 [M + 1]. |
| | yl)imino)(methyl)-λ⁶- | |
| | sulfanone | |
| 143 | (5-fluoropyridin-3- | ¹H-NMR (400 MHz, DMSO-d₆) δ 9.16 (s, 1H), 8.95 (s, 1H), |
| | yl)((2-(5-fluoropyridin- | 8.85-8.81 (m, 1H), 8.68-8.66 (m, 1H), 8.42 (dt, J = 10.0, 2.3 |
| | 3-yl)-2H-pyrazolo[3,4- | Hz, 1H), 8.33 (d, J = 2.7 Hz, 1H), 8.29 (q, J = 4.3 Hz, 1H), |
| | b]pyridin-5- | 8.03 (td, J = 8.6, 2.9 Hz, 1H), 7.43 (d, J = 2.4 Hz, 1H), 3.56 (s, |
| | yl)imino)(methyl)-λ⁶- | 3H); ESI MS(m/z): 386.45 [M + 1]. |
| | sulfanone | |
| 144 | (5-chloropyridin-3- | ¹H-NMR (400 MHz, DMSO-d₆) δ 9.16 (s, 1H), 8.95 (s, 1H), |
| | yl)((2-(5-fluoropyridin- | 8.85-8.81 (m, 1H), 8.68-8.66 (m, 1H), 8.42 (dt, J = 10.0, 2.3 |
| | 3-yl)-2H-pyrazolo[3,4- | Hz, 1H), 8.33 (d, J = 2.7 Hz, 1H), 8.29 (q, J = 4.3 Hz, 1H), |
| | b]pyridin-5- | 8.03 (td, J = 8.6, 2.9 Hz, 1H), 7.43 (d, J = 2.4 Hz, 1H), 3.56 (s, |
| | yl)imino)(methyl)-λ⁶- | 3H); ESI MS(m/z): 402.45 [M + 1]. |
| | sulfanone | |
| 145 | ((2-(5-fluoropyridin-3- | ¹H-NMR (400 MHz, DMSO-d₆) δ 9.33 (d, J = 2.0 Hz, 1H), |
| | yl)-2H-pyrazolo[3,4- | 9.15 (s, 1H), 8.96 (d, J = 3.9 Hz, 1H), 8.67 (dd, J = 6.8, 2.4 |
| | b]pyridin-5- | Hz, 2H), 8.44-8.39 (m, 2H), 8.18 (d, J = 7.6 Hz, 1H), 7.47 (d, |
| | yl)imino)(methyl)(6- | J = 2.7 Hz, 1H), 3.69 (s, 3H); ESI MS(m/z): 437.05 [M + 1]. |
| | (trifluoromethyl)pyridin- | |
| | 3-yl)-λ⁶-sulfanone | |
| 146 | dimethyl((2-(pyridin-3- | ¹H-NMR (400 MHz, DMSO-d₆) δ 9.29 (d, J = 2.4 Hz, 1H), |
| | yl)-2H-pyrazolo[3,4- | 8.98 (s, 1H), 8.64 (dd, J = 4.8, 1.3 Hz, 1H), 8.45 (dq, J = 8.3, |
| | b]pyridin-5-yl)imino)- | 1.4 Hz, 1H), 8.31 (d, J = 2.7 Hz, 1H), 7.65-7.62 (m, 1H), 7.57 |
| | λ⁶-sulfanone | (d, J = 2.4 Hz, 1H), 3.29 (s, 6H); ESI MS(m/z): 287.5 [M + 1]. |
| 147 | ethyl(methyl)((2- | ¹H-NMR (400 MHz, DMSO-d₆) δ 9.29 (d, J = 2.4 Hz, 1H), |
| | (pyridin-3-yl)-2H- | 8.97 (s, 1H), 8.64 (dd, J = 4.8, 1.3 Hz, 1H), 8.44 (dq, J = 8.4, |
| | pyrazolo[3,4-b]pyridin- | 1.3 Hz, 1H), 8.32 (d, J = 2.7 Hz, 1H), 7.65-7.62 (m, 1H), 7.57 |
| | 5-yl)imino)-λ⁶- | (d, J = 2.4 Hz, 1H), 3.48-3.41 (m, 2H), 3.20 (s, 3H), 1.31 (t, |
| | sulfanone | J = 7.3 Hz, 3H); ESI MS(m/z): 301.5 [M + 1]. |
| 148 | ethyl(isopropyl)((2- | ¹H-NMR (400 MHz, DMSO-d₆) δ 9.28 (d, J = 2.4 Hz, 1H), |
| | (pyridin-3-yl)-2H- | 8.96 (s, 1H), 8.64 (dd, J = 4.6, 1.5 Hz, 1H), 8.43 (dq, J = 8.4, |
| | pyrazolo[3,4-b]pyridin- | 1.3 Hz, 1H), 8.34 (dd, J = 11.7, 2.7 Hz, 1H), 7.64 (dd, J = 8.3, |
| | 5-yl)imino)-λ⁶- | 4.9 Hz, 1H), 7.59-7.57 (m, 1H), 3.66-3.56 (m, 1H), 3.40-3.33 |
| | sulfanone | (m, 2H), 1.39 (d, J = 6.6 Hz, 3H), 1.32 (d, J = 6.8 Hz, 3H), |
| | | 1.25 (q, J = 7.1 Hz, 3H); ESI MS(m/z): 329.55 [M + 1]. |
| 149 | dipropyl((2-(pyridin-3- | ¹H-NMR (400 MHz, DMSO-d₆) δ 9.28 (d, J = 2.4 Hz, 1H), |
| | yl)-2H-pyrazolo[3,4- | 8.96 (s, 1H), 8.64 (dd, J = 4.8, 1.3 Hz, 1H), 8.44 (dq, J = 8.3, |
| | b]pyridin-5-yl)imino)- | 1.4 Hz, 1H), 8.32 (d, J = 2.7 Hz, 1H), 7.63 (dd, J = 8.1, 4.4 Hz, |
| | λ⁶-sulfanone | 1H), 7.57 (d, J = 2.7 Hz, 1H), 3.34 (dd, J = 9.7, 6.0 Hz, 4H), |
| | | 1.77 (dt, J = 23.6, 7.4 Hz, 4H), 0.97 (t, J = 7.5 Hz, 6H) |
| | | ESI MS(m/z): 343.55 [M + 1]. |
| 150 | diisobutyl((2-(pyridin- | ¹H-NMR (400 MHz, DMSO-d₆) δ 9.28 (d, J = 2.1 Hz, 1H), |
| | 3-yl)-2H-pyrazolo[3,4- | 8.97 (d, J = 4.0 Hz, 1H), 8.64 (dd, J = 4.7, 1.4 Hz, 1H), 8.44 |
| | b]pyridin-5-yl)imino)- | (dq, J = 8.3, 1.4 Hz, 1H), 8.32 (d, J = 2.8 Hz, 1H), 7.65-7.62 |
| | λ⁶-sulfanone | (m, 1H), 7.56 (d, J = 2.8 Hz, 1H), 3.31-3.24 (m, 4H), 2.36- |
| | | 2.26 (m, 2H), 1.21-0.98 (m, 12H); ESI MS(m/z): 371.65 |
| | | [M + 1]. |
| 151 | (3-methoxypyridin-2- | ¹H-NMR (400 MHz, DMSO-d₆) δ 9.25 (d, J = 2.4 Hz, 1H), |
| | yl)(methyl)((2-(pyridin- | 8.93 (s, 1H), 8.62 (dd, J = 4.8, 1.3 Hz, 1H), 8.40 (dq, J = 8.4, |
| | 3-yl)-2H-pyrazolo[3,4- | 1.3 Hz, 1H), 8.33 (d, J = 2.7 Hz, 1H), 8.23 (dd, J = 4.4, 1.2 Hz, |
| | b]pyridin-5-yl)imino)- | 1H), 7.74 (dd, J = 8.6, 1.2 Hz, 1H), 7.66-7.60 (m, 2H), 7.43 (d, |
| | λ⁶-sulfanone | J = 2.4 Hz, 1H), 3.81 (s, 3H), 3.56 (s, 3H); ESI |
| | | MS(m/z): 380.45 [M + 1]. |
| 152 | (3-fluoropyridin-2- | ¹H-NMR (400 MHz, DMSO-d₆) δ 9.24 (d, J = 2.4 Hz, 1H), |
| | yl)(methyl)((2-(pyridin- | 8.97 (s, 1H), 8.63 (dd, J = 4.6, 1.2 Hz, 1H), 8.58 (d, J = 4.4 |
| | 3-yl)-2H-pyrazolo[3,4- | Hz, 1H), 8.40 (dq, J = 8.4, 1.3 Hz, 1H), 8.32 (d, J = 2.7 Hz, |
| | b]pyridin-5-yl)imino)- | 1H), 8.01 (t, J = 9.4 Hz, 1H), 7.81-7.77 (m, 1H), 7.65-7.60 (m, |
| | λ⁶-sulfanone | 1H), 7.50 (d, J = 2.7 Hz, 1H), 3.66 (s, 3H); ESI |
| | | MS(m/z): 368.45 [M + 1]. |
| 153 | (2-chloropyridin-3- | ¹H-NMR (400 MHz, DMSO-d₆) δ 9.19 (d, J = 2.4 Hz, 1H), |
| | yl)(methyl)((2-(pyridin- | 8.88 (t, J = 3.8 Hz, 1H), 8.62-8.59 (m, 2H), 8.56 (dd, J = 7.6, |
| | 3-yl)-2H-pyrazolo[3,4- | 1.8 Hz, 1H), 8.37-8.34 (m, 2H), 7.68 (dd, J = 7.8, 4.7 Hz, 1H), |
| | b]pyridin-5-yl)imino)- | 7.58 (ddd, J = 8.4, 4.7, 0.6 Hz, 1H), 7.36 (d, J = 2.8 Hz, 1H), |
| | λ⁶-sulfanone | 3.68 (s, 3H); ESI MS(m/z): 384.5 [M + 1]. |
| 154 | (5-chloropyridin-3- | ¹H-NMR (400 MHz, DMSO-d₆) δ 9.24 (dd, J = 7.3, 2.8 Hz, |
| | yl)(methyl)((2-(pyridin- | 1H), 9.07 (d, J = 1.8 Hz, 1H), 8.93-8.88 (m, 2H), 8.64-8.61 |
| | 3-yl)-2H-pyrazolo[3,4- | (m, 1H), 8.52 (t, J = 2.1 Hz, 1H), 8.41-8.37 (m, 2H), 7.64-7.60 |
| | b]pyridin-5-yl)imino)- | (m, 1H), 7.46 (d, J = 2.8 Hz, 1H), 3.65 (d, J = 8.3 Hz, 3H) |
| | λ⁶-sulfanone | ESI MS(m/z): 384.45 [M + 1]. |
| 155 | 1-((2-(pyridin-3-yl)-2H- | ¹H-NMR (400 MHz, DMSO-d₆) δ 9.29 (d, J = 2.4 Hz, 1H), |
| | pyrazolo[3,4-b]pyridin- | 8.98 (d, J = 4.0 Hz, 1H), 8.64 (dd, J = 4.7, 1.4 Hz, 1H), 8.45 |
| | 5-yl)imino)tetrahydro- | (dq, J = 8.4, 1.3 Hz, 1H), 8.33 (d, J = 2.8 Hz, 1H), 7.66-7.62 |
| | 1H-λ⁶-thiophene 1- | (m, 1H), 7.51 (d, J = 2.4 Hz, 1H), 3.50-3.41 (m, 2H), 3.34 (s, |
| | oxide | 1H), 3.29 (t, J = 7.0 Hz, 1H), 2.32-2.19 (m, 2H), 2.18-2.08 (m, |
| | | 2H); ESI MS(m/z): 313.5 [M + 1]. |
| 156 | isopropyl(methyl)((2- | ¹H-NMR (400 MHz, DMSO-d₆) δ 9.29 (d, J = 2.4 Hz, 1H), |
| | (pyridin-3-yl)-2H- | 8.97 (s, 1H), 8.64 (dd, J = 4.8, 1.3 Hz, 1H), 8.44 (dq, J = 8.4, |
| | pyrazolo[3,4-b]pyridin- | 1.3 Hz, 1H), 8.33 (d, J = 2.7 Hz, 1H), 7.64 (dd, J = 8.6, 5.1 Hz, |
| | 5-yl)imino)-λ⁶- | 1H), 7.59 (d, J = 2.4 Hz, 1H), 3.58 (t, J = 6.8 Hz, 1H), 3.12 (s, |
| | sulfanone | 3H), 1.41 (d, J = 6.8 Hz, 3H), 1.37 (d, J = 6.8 Hz, 3H) |
| | | ESI MS(m/z): 315.5 [M + 1]. |
| 157 | methyl((2-(pyridin-3- | ¹H-NMR (400 MHz, DMSO-d₆) δ 9.33 (d, J = 2.1 Hz, 1H), |
| | yl)-2H-pyrazolo[3,4- | 9.22 (d, J = 2.4 Hz, 1H), 8.91 (d, J = 4.0 Hz, 1H), 8.68-8.62 |
| | b]pyridin-5- | (m, 2H), 8.42 (d, J = 2.8 Hz, 1H), 8.39 (dq, J = 8.3, 1.4 Hz, |
| | yl)imino)(6- | 1H), 8.18 (d, J = 8.3 Hz, 1H), 7.63-7.60 (m, 1H), 7.48 (d, J = |
| | (trifluoromethyl)pyridin- | 2.8 Hz, 1H), 3.67 (d, J = 14.4 Hz, 3H); ESI MS(m/z): 418.5 |
| | 3-yl)-λ⁶-sulfanone | [M + 1]. |
| 158 | methyl(pyridin-2- | ¹H-NMR (400 MHz, DMSO-d₆) δ 9.23 (d, J = 2.4 Hz, 1H), |
| | yl)((2-(pyridin-3-yl)- | 8.91 (s, 1H), 8.78 (d, J = 3.9 Hz, 1H), 8.62 (dd, J = 4.6, 1.2 |
| | 2H-pyrazolo[3,4- | Hz, 1H), 8.39 (dq, J = 8.3, 1.4 Hz, 1H), 8.30 (d, J = 2.4 Hz, |
| | b]pyridin-5-yl)imino)- | 1H), 8.18 (d, J = 7.8 Hz, 1H), 8.11 (td, J = 7.7, 1.7 Hz, 1H), |
| | λ⁶-sulfanone | 7.68 (ddd, J = 7.5, 4.7, 1.2 Hz, 1H), 7.61 (dd, J = 8.1, 4.6 Hz, |
| | | 1H), 7.43 (d, J = 2.7 Hz, 1H), 3.55 (s, 3H); ESI MS(m/z): |
| | | 350.5 [M + 1]. |
| 159 | (5-fluoropyridin-3- | ¹H-NMR (400 MHz, DMSO-d₆) δ 9.23 (d, J = 2.2 Hz, 1H), |
| | yl)(methyl)((2-(pyridin- | 8.91 (s, 1H), 8.82 (d, J = 2.8 Hz, 1H), 8.63 (dd, J = 4.8, 1.3 |
| | 3-yl)-2H-pyrazolo[3,4- | Hz, 1H), 8.39 (dq, J = 8.3, 1.4 Hz, 1H), 8.31 (d, J = 2.4 Hz, |
| | b]pyridin-5-yl)imino)- | 1H), 8.27 (q, J = 4.4 Hz, 1H), 8.03 (td, J = 8.7, 2.9 Hz, 1H), |
| | λ⁶-sulfanone | 7.62 (ddd, J = 8.3, 4.8, 0.6 Hz, 1H), 7.44 (d, J = 2.7 Hz, 1H), |
| | | 5.75 (s, 1H), 3.56 (s, 2H); ESI MS(m/z): 368.45 [M + 1]. |
| 160 | 1-((2-(5-chloropyridin- | ¹H-NMR (400 MHz, DMSO-d₆) δ 9.26 (d, J = 2.0 Hz, 1H), |
| | 3-yl)-2H-indazol-5- | 8.99 (d, J = 0.7 Hz, 1H), 8.66 (d, J = 2.0 Hz, 1H), 8.60 (t, J = |
| | yl)imino)tetrahydro- | 2.2 Hz, 1H), 7.58 (d, J = 9.0 Hz, 1H), 7.08 (q, J = 0.9 Hz, 1H), |
| | 1H-λ⁶-thiophene 1- | 7.00 (dd, J = 9.2, 2.1 Hz, 1H), 3.41-3.35 (m, 2H), 3.28-3.21 |
| | oxide | (m, 2H), 2.24-2.18 (m, 2H), 2.14-2.06 (m, 2H) |
| | | ESI MS(m/z): 368.45 [M + 1]. |
| 161 | 1-((2-(2-methylpyridin- | ¹H-NMR (400 MHz, DMSO-d₆) δ 8.60 (dd, J = 4.8, 1.6 Hz, |
| | 3-yl)-2H-indazol-5- | 1H), 8.48 (d, J = 0.7 Hz, 1H), 7.91 (dd, J = 7.8, 1.5 Hz, 1H), |
| | yl)imino)tetrahydro- | 7.57 (d, J = 9.0 Hz, 1H), 7.46 (dd, J = 7.7, 4.8 Hz, 1H), 7.14 |
| | 1H-λ⁶-thiophene 1- | (d, J = 1.2 Hz, 1H), 6.98 (dd, J = 9.2, 2.1 Hz, 1H), 3.41-3.34 |
| | oxide | (m, 2H), 3.25-3.18 (m, 2H), 2.42 (s, 3H), 2.24-2.19 (m, 2H), |
| | | 2.15-2.07 (m, 3H); ESI MS(m/z): 326.95 [M + 1]. |
| 162 | 1-((2-(4-methylpyridin- | ¹H-NMR (400 MHz, DMSO-d₆) δ 8.65 (s, 1H), 8.56 (d, J = |
| | 3-yl)-2H-indazol-5- | 4.9 Hz, 1H), 8.50 (d, J = 1.0 Hz, 1H), 7.57 (d, J = 9.3 Hz, 1H), |
| | yl)imino)tetrahydro- | 7.51 (d, J = 5.1 Hz, 1H), 7.15 (d, J = 1.5 Hz, 1H), 6.98 (dd, J = |
| | 1H-λ⁶-thiophene 1- | 9.2, 2.1 Hz, 1H), 3.41-3.34 (m, 2H), 3.25-3.18 (m, 2H), 2.29 |
| | oxide | (s, 3H), 2.24-2.18 (m, 2H), 2.15-2.08 (m, 2H); ESI |
| | | MS(m/z): 326.95 [M + 1]. |
| 163 | 1-((2-(6-methylpyridin- | ¹H-NMR (400 MHz, DMSO-d₆) δ 9.11 (d, J = 2.4 Hz, 1H), |
| | 3-yl)-2H-indazol-5- | 8.87 (d, J = 0.7 Hz, 1H), 8.28 (dd, J = 8.6, 2.7 Hz, 1H), 7.58 |
| | yl)imino)tetrahydro- | (d, J = 9.0 Hz, 1H), 7.45 (d, J = 8.3 Hz, 1H), 7.10 (t, J = 1.0 |
| | 1H-λ⁶-thiophene 1- | Hz, 1H), 6.97 (dd, J = 9.2, 2.1 Hz, 1H), 3.41-3.34 (m, 2H), |
| | oxide | 3.26-3.16 (m, 2H), 2.54 (s, 3H), 2.27-2.05 (m, 4H); ESI |
| | | MS(m/z): 327.05 [M + 1]. |
| 164 | 1-((2-(5-methylpyridin- | H-NMR (400 MHz, DMSO-d₆) δ 9.06 (d, J = 2.4 Hz, 1H), |
| | 3-yl)-2H-indazol-5- | 8.90 (d, J = 1.0 Hz, 1H), 8.45 (d, J = 1.0 Hz, 1H), 8.27-8.26 |
| | yl)imino)tetrahydro- | (m, 1H), 7.58 (dt, J = 9.1, 0.9 Hz, 1H), 7.10 (q, J = 0.9 Hz, |
| | 1H-λ⁶-thiophene 1- | 1H), 6.98 (dd, J = 9.2, 2.1 Hz, 1H), 3.41-3.34 (m, 2H), 3.26- |
| | oxide | 3.20 (m, 2H), 2.42 (d, J = 0.5 Hz, 3H), 2.26-2.07 (m, 4H); ESI |
| | | MS(m/z): 326.65 [M + 1]. |
| 165 | ((2-(5-chloropyridin-3- | ¹H-NMR (400 MHz, DMSO-d₆) δ 9.26 (d, J = 2.4 Hz, 1H), |
| | yl)-2H-indazol-5- | 8.98 (d, J = 0.6 Hz, 1H), 8.66 (d, J = 2.1 Hz, 1H), 8.60 (t, J = |
| | yl)imino)dimethyl-λ⁶- | 2.3 Hz, 1H), 7.57 (d, J = 9.2 Hz, 1H), 7.14 (q, J = 0.9 Hz, 1H), |
| | sulfanone | 6.98 (dd, J = 9.2, 2.1 Hz, 1H), 3.23 (s, 6H); ESI |
| | | MS(m/z): 320.9 [M + 1]. |
| 166 | dimethyl((2-(5- | ¹H-NMR (400 MHz, DMSO-d₆) δ 9.60 (d, J = 2.4 Hz, 1H), |
| | (trifluoromethyl)pyridin- | 9.10 (d, J = 0.9 Hz, 1H), 9.00 (d, J = 0.9 Hz, 1H), 8.78 (t, J = |
| | 3-yl)-2H-indazol-5- | 1.8 Hz, 1H), 7.59 (d, J = 9.2 Hz, 1H), 7.15 (t, J = 0.9 Hz, 1H), |
| | yl)imino)-λ⁶-sulfanone | 7.00 (dd, J = 9.3, 2.0 Hz, 1H), 3.24 (s, 6H); ESI |
| | | MS(m/z): 354.85 [M + 1]. |
| 167 | ((2-(5-methoxypyridin- | ¹H-NMR (400 MHz, DMSO-d₆) δ 8.96 (d, J = 0.9 Hz, 1H), |
| | 3-yl)-2H-indazol-5- | 8.87 (d, J = 2.1 Hz, 1H), 8.33 (d, J = 2.4 Hz, 1H), 7.99 (t, J = |
| | yl)imino)dimethyl-λ⁶- | 2.3 Hz, 1H), 7.59-7.56 (m, 1H), 7.15-7.14 (m, 1H), 6.97 (dd, |
| | sulfanone | J = 9.2, 2.1 Hz, 1H), 3.94 (s, 3H), 3.23 (s, 6H); ESI |
| | | MS(m/z): 316.5 [M + 1]. |
| 168 | dimethyl((2-(5- | ¹H-NMR (400 MHz, DMSO-d₆) δ 9.06 (d, J = 2.4 Hz, 1H), |
| | methylpyridin-3-yl)- | 8.90 (d, J = 0.7 Hz, 1H), 8.45 (d, J = 1.0 Hz, 1H), 8.27-8.26 |
| | 2H-indazol-5- | (m, 1H), 7.56 (dt, J = 9.1, 0.9 Hz, 1H), 7.16-7.15 (m, 1H), |
| | yl)imino)-λ⁶-sulfanone | 6.96 (dd, J = 9.2, 2.1 Hz, 1H), 3.22 (s, 6H), 2.42 (d, J = 0.5 |
| | | Hz, 3H); ESI MS(m/z): 300.55 [M + 1]. |
| 169 | 1-((2-(2,6- | ¹H-NMR (400 MHz, DMSO-d₆) δ 8.43 (d, J = 0.9 Hz, 1H), |
| | dimethylpyridin-3-yl)- | 7.77 (d, J = 7.9 Hz, 1H), 7.56 (d, J = 9.2 Hz, 1H), 7.29 (d, J = |
| | 2H-indazol-5- | 8.3 Hz, 1H), 7.14 (q, J = 0.9 Hz, 1H), 6.97 (dd, J = 9.0, 2.0 Hz, |
| | yl)imino)tetrahydro- | 1H), 3.41-3.34 (m, 2H), 3.24-3.18 (m, 2H), 2.53 (s, 3H), 2.35 |
| | 1H-λ⁶-thiophene 1- | (s, 3H), 2.25-2.19 (m, 2H), 2.16-2.05 (m, 2H); ESI |
| | oxide | MS(m/z): 341 [M + 1]. |
| 170 | 1-((2-(5- | ¹H-NMR (400 MHz, DMSO-d₆) δ 8.96 (d, J = 0.9 Hz, 1H), |
| | methoxypyridin-3-yl)- | 8.88 (d, J = 1.8 Hz, 1H), 8.33 (d, J = 2.4 Hz, 1H), 7.99 (t, J = |
| | 2H-indazol-5- | 2.4 Hz, 1H), 7.59 (d, J = 9.2 Hz, 1H), 7.09 (q, J = 0.9 Hz, 1H), |
| | yl)imino)tetrahydro- | 6.98 (dd, J = 9.2, 1.8 Hz, 1H), 3.98-3.91 (m, 3H), 3.41-3.35 |
| | 1H-λ⁶-thiophene 1- | (m, 2H), 3.27-3.20 (m, 2H), 2.28-2.05 (m, 5H); ESI |
| | oxide | MS(m/z): 342.95 [M + 1]. |
| 171 | 1-((2-(5- | ¹H-NMR (400 MHz, DMSO-d₆) δ 9.60 (d, J = 2.4 Hz, 1H), |
| | (trifluoromethyl)pyridin- | 9.10 (d, J = 0.9 Hz, 1H), 9.00 (d, J = 0.9 Hz, 1H), 8.79 (t, J = |
| | 3-yl)-2H-indazol-5- | 1.8 Hz, 1H), 7.61 (d, J = 9.5 Hz, 1H), 7.09 (q, J = 0.9 Hz, 1H), |
| | yl)imino)tetrahydro- | 7.01 (dd, J = 9.2, 1.8 Hz, 1H), 3.42-3.35 (m, 2H), 3.29-3.22 |
| | 1H-λ⁶-thiophene 1- | (m, 2H), 2.26-2.06 (m, 4H); ESI MS(m/z): 380.85 [M + 1]. |
| | oxide | |
| 173 | dimethyl((2-(2- | ¹H-NMR (400 MHz, DMSO-d₆) δ 8.60 (dd, J = 4.9, 1.5 Hz, |
| | methylpyridin-3-yl)- | 1H), 8.48 (d, J = 0.9 Hz, 1H), 7.91 (dd, J = 7.9, 1.5 Hz, 1H), |
| | 2H-indazol-5- | 7.56 (d, J = 9.2 Hz, 1H), 7.45 (q, J = 4.1 Hz, 1H), 7.19 (q, J = |
| | yl)imino)-λ⁶-sulfanone | 0.9 Hz, 1H), 6.97 (dd, J = 9.2, 1.8 Hz, 1H), 3.22 (s, 6H), 2.43 |
| | | (s, 3H); ESI MS(m/z): 300.55 [M + 1]. |
| 174 | dimethyl((2-(6- | ¹H-NMR (400 MHz, DMSO-d₆) δ 9.11 (d, J = 2.4 Hz, 1H), |
| | methylpyridin-3-yl)- | 8.87 (d, J = 0.7 Hz, 1H), 8.28 (dd, J = 8.3, 2.7 Hz, 1H), 7.56 |
| | 2H-indazol-5- | (dt, J = 9.1, 0.9 Hz, 1H), 7.45 (d, J = 8.6 Hz, 1H), 7.15 (q, J = |
| | yl)imino)-λ⁶-sulfanone | 0.9 Hz, 1H), 6.96 (dd, J = 9.2, 2.1 Hz, 1H), 3.22 (s, 6H), 2.52 |
| | | (d, J = 9.5 Hz, 3H); ESI MS(m/z): 300.5 [M + 1]. |
| 175 | dimethyl((2-(4- | ¹H-NMR (400 MHz, DMSO-d₆) δ 8.64 (s, 1H), 8.56 (d, J = |
| | methylpyridin-3-yl)- | 4.9 Hz, 1H), 8.49 (d, J = 0.9 Hz, 1H), 7.57-7.55 (m, 1H), 7.51 |
| | 2H-indazol-5- | (d, J = 4.9 Hz, 1H), 7.19 (q, J = 0.9 Hz, 1H), 6.97 (dd, J = 9.2, |
| | yl)imino)-λ⁶-sulfanone | 2.1 Hz, 1H), 3.22 (s, 6H), 2.30 (d, J = 5.5 Hz, 3H) |
| | | ESI MS(m/z): 300.55 [M + 1]. |
| 176 | ((2-(2,6- | ¹H-NMR (400 MHz, DMSO-d₆) δ 8.42 (d, J = 0.9 Hz, 1H), |
| | dimethylpyridin-3-yl)- | 7.77 (d, J = 7.9 Hz, 1H), 7.55 (dt, J = 9.2, 0.9 Hz, 1H), 7.29 (d, |
| | 2H-indazol-5- | J = 7.9 Hz, 1H), 7.18 (q, J = 0.9 Hz, 1H), 6.96 (dd, J = 9.2, 2.1 |
| | yl)imino)dimethyl-λ⁶- | Hz, 1H), 3.21 (s, 6H), 2.53 (s, 3H), 2.36 (s, 3H); ESI |
| | sulfanone | MS(m/z): 314.95 [M + 1]. |
| 177 | ((3-chloro-2- | ¹H-NMR (400 MHz, DMSO-d₆) δ 9.37 (s, 1H), 9.29 (s, 2H), |
| | (pyrimidin-5-yl)-2H- | 7.61 (dd, J = 9.3, 0.8 Hz, 1H), 7.05 (dd, J = 9.2, 2.1 Hz, 1H), |
| | indazol-5- | 6.99 (q, J = 0.9 Hz, 1H), 3.28-3.22 (m, 6H); ESI |
| | yl)imino)dimethyl-λ⁶- | MS(m/z): 321.7 [M + 1]. |
| | sulfanone | |
| 178 | ((3-chloro-2- | ¹H-NMR (400 MHz, DMSO-d₆) δ 9.33 (d, J = 32.7 Hz, 2H), |
| | (pyrimidin-5-yl)-2H- | 7.61-7.59 (m, 1H), 7.08 (dd, J = 9.2, 2.1 Hz, 1H), 7.02 (q, J = |
| | indazol-5- | 0.9 Hz, 1H), 3.56 (s, 1H), 3.08 (s, 3H), 1.40 (d, J = 6.7 Hz, |
| | yl)imino)(isopropyl)(methyl)- | 3H), 1.35 (d, J = 7.0 Hz, 3H); ESI MS(m/z): 349.95 [M + 1]. |
| | λ⁶-sulfanone | |
| 179 | 1-((3-chloro-2- | ¹H-NMR (400 MHz, DMSO-d₆) δ 9.37 (s, 1H), 9.29 (s, 2H), |
| | (pyrimidin-5-yl)-2H- | 7.62 (dd, J = 9.2, 0.9 Hz, 1H), 7.06 (dd, J = 9.2, 1.8 Hz, 1H), |
| | indazol-5- | 6.93 (q, J = 0.9 Hz, 1H), 3.43-3.36 (m, 2H), 3.27 (t, J = 7.0 |
| | yl)imino)tetrahydro- | Hz, 1H), 2.28-2.19 (m, 2H), 2.15-2.08 (m, 2H); ESI |
| | 1H-λ⁶-thiophene 1- | MS(m/z): 347.9 [M + 1]. |
| | oxide | |
| 180 | ((3-chloro-2- | ¹H-NMR (400 MHz, DMSO-d₆) δ 9.37 (s, 1H), 9.29 (s, 2H), |
| | (pyrimidin-5-yl)-2H- | 7.60 (dd, J = 9.2, 0.9 Hz, 1H), 7.06 (dd, J = 9.2, 2.1 Hz, 1H), |
| | indazol-5- | 7.00 (q, J = 0.9 Hz, 1H), 3.37 (dd, J = 9.9, 6.0 Hz, 2H), 3.17 |
| | yl)imino)(methyl)(propyl)- | (s, 3H), 1.79 (dd, J = 16.8, 7.5 Hz, 2H), 0.98 (t, J = 7.3 Hz, |
| | λ⁶-sulfanone | 3H); ESI MS(m/z): 349.9 [M + 1]. |
| 181 | ((3-chloro-2- | ¹H-NMR (400 MHz, DMSO-d₆) δ 9.37 (s, 1H), 9.30-9.26 (m, |
| | (pyrimidin-5-yl)-2H- | 2H), 7.59 (dd, J = 9.2, 0.6 Hz, 1H), 7.07 (dd, J = 9.2, 1.8 Hz, |
| | indazol-5- | 1H), 7.00 (q, J = 0.9 Hz, 1H), 3.28-3.21 (m, 4H), 2.34-2.27 |
| | yl)imino)diisobutyl-λ⁶- | (m, 2H), 1.06-1.00 (m, 12H); ESI MS(m/z): 406.05 [M + 1]. |
| | sulfanone | |
| 184 | dipropyl((2-(pyrimidin- | ¹H-NMR (400 MHz, DMSO-d₆) δ 9.49 (s, 2H), 9.25 (s, 1H), |
| | 5-yl)-2H-pyrazolo[3,4- | 9.02 (s, 1H), 8.34 (d, J = 2.4 Hz, 1H), 7.57 (d, J = 2.7 Hz, 1H), |
| | b]pyridin-5-yl)imino)- | 3.38-3.34 (m, 4H), 1.77 (dt, J = 23.6, 7.2 Hz, 4H), 0.97 (t, J = |
| | λ⁶-sulfanone | 7.5 Hz, 6H); ESI MS(m/z): 345.05 [M + 1]. |
| 185 | diisobutyl((2- | ¹H-NMR (400 MHz, DMSO-d₆) δ 9.51 (d, J = 10.0 Hz, 2H), |
| | (pyrimidin-5-yl)-2H- | 9.25 (t, J = 4.9 Hz, 1H), 9.02 (d, J = 4.6 Hz, 1H), 8.34 (d, J = |
| | pyrazolo[3,4-b]pyridin- | 2.7 Hz, 1H), 7.57 (d, J = 2.7 Hz, 1H), 3.35 (d, J = 6.1 Hz, 1H), |
| | 5-yl)imino)-λ⁶- | 3.28 (d, J = 7.8 Hz, 1H), 2.36-2.26 (m, 2H), 1.06-0.98 (m, |
| | sulfanone | 12H); ESI MS(m/z): 373.05 [M + 1]. |
| 186 | 1-((2-(pyrimidin-5-yl)- | ¹H-NMR (400 MHz, DMSO-d₆) δ 9.51 (s, 2H), 9.25 (s, 1H), |
| | 2H-pyrazolo[3,4- | 9.04 (s, 1H), 8.36 (d, J = 2.4 Hz, 1H), 7.52 (d, J = 2.7 Hz, 1H), |
| | b]pyridin-5- | 3.48-3.42 (m, 2H), 3.35 (d, J = 7.1 Hz, 1H), 3.29 (s, 1H), 2.26- |
| | yl)imino)tetrahydro- | 2.10 (m, 4H); ESI MS(m/z): 314.85[M + 1]. |
| | 1H-λ⁶-thiophene 1- | |
| | oxide | |
| 187 | ((2-(5-fluoropyridin-3- | ¹H-NMR (400 MHz, DMSO-d₆) δ 9.22 (s, 1H), 9.02 (s, 1H), |
| | yl)-2H-pyrazolo[3,4- | 8.68 (d, J = 2.4 Hz, 1H), 8.48 (dt, J = 10.1, 2.4 Hz, 1H), 8.32 |
| | b]pyridin-5- | (d, J = 2.4 Hz, 1H), 7.55 (d, J = 2.7 Hz, 1H), 3.30 (s, 6H) |
| | yl)imino)dimethyl-16- | ESI MS(m/z): 305.905[M + 1]. |
| | sulfanone | |
| 188 | 1-((2-(5-fluoropyridin- | ¹H-NMR (400 MHz, DMSO-d₆) δ 9.22 (s, 1H), 9.02 (s, 1H), |
| | 3-yl)-2H-pyrazolo[3,4- | 8.68 (d, J = 2.4 Hz, 1H), 8.48 (dt, J = 10.0, 2.3 Hz, 1H), 8.35 |
| | b]pyridin-5- | (d, J = 2.7 Hz, 1H), 7.50 (d, J = 2.7 Hz, 1H), 3.48-3.41 (m, |
| | yl)imino)tetrahydro- | 2H), 3.34 (d, J = 7.3 Hz, 1H), 3.28 (s, 1H), 2.26-2.20 (m, 2H), |
| | 1H-λ⁶-thiophene 1- | 2.17-2.11 (m, 2H); ESI MS(m/z): 376.9[M + 1]. |
| | oxide | |
| 189 | ((3-chloro-2-(pyridin-3- | ¹H-NMR (400 MHz, DMSO-d₆) δ 8.92 (d, J = 2.1 Hz, 1H), |
| | yl)-2H-indazol-5- | 8.74 (dd, J = 4.9, 1.5 Hz, 1H), 8.20-8.17 (m, 1H), 7.67 (dd, J = |
| | yl)imino)dipropyl-λ⁶- | 7.9, 4.6 Hz, 1H), 7.56 (d, J = 9.2 Hz, 1H), 7.05 (dd, J = 9.3, |
| | sulfanone | 2.0 Hz, 1H), 6.99 (d, J = 1.2 Hz, 1H), 3.30-3.26 (m, 4H), 1.78- |
| | | 1.70 (m, 4H), 0.96 (t, J = 7.5 Hz, 6H); ESI MS(m/z): |
| | | 320.8[M + 1]. |
| 190 | ((3-chloro-2-(pyridin-3- | ¹H-NMR (400 MHz, DMSO-d₆) δ 8.95 (d, J = 2.0 Hz, 1H), |
| | yl)-2H-indazol-5- | 8.76 (dd, J = 4.6, 1.5 Hz, 1H), 8.22-8.19 (m, 1H), 7.68 (dd, J = |
| | yl)imino)dimethyl-λ⁶- | 8.2, 4.8 Hz, 1H), 7.59 (dd, J = 9.2, 0.9 Hz, 1H), 7.03 (dd, J = |
| | sulfanone | 9.3, 2.0 Hz, 1H), 6.99 (t, J = 1.0 Hz, 1H), 3.25 (s, 6H) |
| | | ESI MS(m/z): 331.9[M + 1]. |
| 191 | 1-((3-chloro-2-(pyridin- | ¹H-NMR (400 MHz, DMSO-d₆) δ 8.95-8.95 (m, 1H), 8.76 |
| | 3-yl)-2H-indazol-5- | (dd, J = 4.7, 1.4 Hz, 1H), 8.21 (dq, J = 8.3, 1.4 Hz, 1H), 7.68 |
| | yl)imino)tetrahydro- | (ddd, J = 8.3, 4.9, 0.6 Hz, 1H), 7.60 (dd, J = 9.3, 0.8 Hz, 1H), |
| | 1H-λ⁶-thiophene 1- | 7.04 (dd, J = 9.3, 2.0 Hz, 1H), 6.93 (q, J = 0.9 Hz, 1H), 3.39 |
| | oxide | (td, J = 13.1, 6.6 Hz, 2H), 3.26 (t, J = 7.2 Hz, 2H), 2.28-2.19 |
| | | (m, 2H), 2.15-2.08 (m, 2H); ESI MS(m/z): 346.85[M + 1]. |
| 192 | ((3-chloro-2-(5- | ¹H-NMR (400 MHz, DMSO-d₆) δ 8.89 (t, J = 1.4 Hz, 1H), |
| | fluoropyridin-3-yl)-2H- | 8.82 (d, J = 3.1 Hz, 1H), 8.30 (dt, J = 9.5, 2.3 Hz, 1H), 7.57 |
| | indazol-5- | (dd, J = 9.2, 0.9 Hz, 1H), 7.06 (dd, J = 9.3, 2.0 Hz, 1H), 6.99 |
| | yl)imino)dipropyl-λ⁶- | (q, J = 0.9 Hz, 1H), 3.35 (s, 1H), 3.29 (s, 3H), 1.81-1.71 (m, |
| | sulfanone | 4H), 0.98 (q, J = 7.5 Hz, 6H); ESI MS(m/z): 394.9[M + 1]. |
| 193 | ethyl(isopropyl)((2- | ¹H-NMR (400 MHz, DMSO-d₆) δ 9.49 (s, 2H), 9.25 (s, 1H), |
| | (pyrimidin-5-yl)-2H- | 9.01 (s, 1H), 8.38 (d, J = 2.4 Hz, 1H), 7.59 (d, J = 2.4 Hz, 1H), |
| | pyrazolo[3,4-b]pyridin- | 3.62 (t, J = 6.9 Hz, 1H), 3.37 (q, J = 7.3 Hz, 2H), 1.39 (d, J = |
| | 5-ylimino)-λ⁶- | 6.7 Hz, 3H), 1.32 (d, J = 6.7 Hz, 3H), 1.26 (t, J = 7.3 Hz, 3H); |
| | sulfanone | ESI MS(m/z): 331.05[M + 1]. |
| 194 | butyl((3-chloro-2-(5- | ¹H-NMR (400 MHz, DMSO-d₆) δ 8.89 (d, J = 1.2 Hz, 1H), |
| | fluoropyridin-3-yl)-2H- | 8.82 (d, J = 2.8 Hz, 1H), 8.31 (dt, J = 9.4, 2.3 Hz, 1H), 7.58 |
| | indazol-5- | (dd, J = 9.2, 0.9 Hz, 1H), 7.07 (dd, J = 9.2, 1.8 Hz, 1H), 6.99 |
| | yl)imino)(ethyl)-λ⁶- | (q, J = 0.9 Hz, 1H), 3.36 (t, J = 7.5 Hz, 3H), 3.30 (s, 1H), 1.76- |
| | sulfanone | 1.68 (m, 2H), 1.39 (td, J = 14.7, 7.4 Hz, 2H), 1.29-1.22 (m, |
| | | 3H), 0.88 (t, J = 7.3 Hz, 3H); ESI MS(m/z): 394.9[M + 1]. |
| 195 | butyl((3-chloro-2- | ¹H-NMR (400 MHz, DMSO-d₆) δ 8.95 (d, J = 2.0 Hz, 1H), |
| | (pyridin-3-yl)-2H- | 8.75 (dd, J = 4.7, 1.4 Hz, 1H), 8.20 (dq, J = 8.2, 1.3 Hz, 1H), |
| | indazol-5- | 7.70-7.66 (m, 1H), 7.58-7.56 (m, 1H), 7.06 (dd, J = 9.2, 1.8 |
| | yl)imino)(ethyl)-λ⁶- | Hz, 1H), 7.00 (t, J = 0.9 Hz, 1H), 3.35 (d, J = 7.6 Hz, 2H), |
| | sulfanone | 3.27-3.23 (m, 2H), 1.76-1.68 (m, 2H), 1.39 (td, J = 14.8, 7.5 |
| | | Hz, 2H), 1.26 (t, J = 7.5 Hz, 3H), 0.88 (t, J = 7.3 Hz, 3H); ESI |
| | | MS(m/z): 377.1[M + 1]. |
| 196 | ((3-chloro-2-(pyridin-3- | ¹H-NMR (400 MHz, DMSO-d₆) δ 8.95 (d, J = 2.1 Hz, 1H), |
| | yl)-2H-indazol-5- | 8.75 (dd, J = 4.7, 1.4 Hz, 1H), 8.20 (dq, J = 8.2, 1.3 Hz, 1H), |
| | yl)imino)(ethyl)(isopropyl)- | 7.68 (ddd, J = 8.3, 4.9, 0.6 Hz, 1H), 7.57 (dd, J = 9.2, 0.6 Hz, |
| | λ⁶-sulfanone | 1H), 7.09 (dd, J = 9.3, 2.0 Hz, 1H), 7.02 (q, J = 0.9 Hz, 1H), |
| | | 3.60-3.54 (m, 1H), 3.35-3.32 (m, 1H), 3.29 (d, J = 7.6 Hz, |
| | | 1H), 1.38 (d, J = 6.7 Hz, 3H), 1.31 (d, J = 6.7 Hz, 3H), 1.25 (t, |
| | | J = 7.5 Hz, 3H); ESI MS(m/z): 363[M + 1]. |
| 197 | ((3-chloro-2-(pyridin-3- | ¹H-NMR (400 MHz, DMSO-d₆) δ 8.95 (d, J = 2.2 Hz, 1H), |
| | yl)-2H-indazol-5- | 8.75 (dd, J = 4.8, 1.3 Hz, 1H), 8.20 (dq, J = 8.3, 1.3 Hz, 1H), |
| | yl)imino)(methyl)(propyl)- | 7.68 (dd, J = 7.8, 5.1 Hz, 1H), 7.59-7.57 (m, 1H), 7.04 (dd, J = |
| | λ⁶-sulfanone | 9.2, 2.1 Hz, 1H), 7.00 (d, J = 1.2 Hz, 1H), 3.37 (dd, J = 9.8, |
| | | 6.1 Hz, 2H), 3.16 (s, 3H), 1.82-1.74 (m, 2H), 1.00-0.91 (m, |
| | | 3H); ESI MS(m/z): 348.95[M + 1]. |
| 198 | ((3-chloro-2-(pyridin-3- | ¹H-NMR (400 MHz, DMSO-d₆) δ 8.95 (d, J = 2.1 Hz, 1H), |
| | yl)-2H-indazol-5- | 8.75 (dd, J = 4.7, 1.4 Hz, 1H), 8.20 (dq, J = 8.3, 1.3 Hz, 1H), |
| | yl)imino)(isopropyl)(methyl)- | 7.72-7.66 (m, 1H), 7.58 (dd, J = 9.2, 0.9 Hz, 1H), 7.06 (dd, J = |
| | λ⁶-sulfanone | 9.2, 2.1 Hz, 1H), 7.01 (q, J = 0.9 Hz, 1H), 3.58-3.52 (m, 1H), |
| | | 3.07 (s, 3H), 1.40 (d, J = 6.7 Hz, 3H), 1.35 (d, J = 7.0 Hz, 3H) |
| | | ESI MS(m/z): 348.95[M + 1]. |
| 199 | ((3-chloro-2-(5- | ¹H-NMR (400 MHz, DMSO-d₆) δ 8.89 (s, 1H), 8.82 (d, J = |
| | fluoropyridin-3-yl)-2H- | 2.8 Hz, 1H), 8.30 (dt, J = 9.4, 2.3 Hz, 1H), 7.57 (dd, J = 9.2, |
| | indazol-5- | 0.6 Hz, 1H), 7.11 (dd, J = 9.2, 1.8 Hz, 1H), 7.01 (q, J = 0.9 Hz, |
| | yl)imino)(ethyl)(isopropyl)- | 1H), 3.61-3.54 (m, 1H), 3.33 (d, J = 7.3 Hz, 1H), 3.29 (s, 1H), |
| | λ⁶-sulfanone | 1.38 (d, J = 6.7 Hz, 3H), 1.34-1.29 (m, 3H), 1.27-1.21 (m, 3H) |
| | | ESI MS(m/z): 380.95[M + 1]. |
| 200 | ((3-chloro-2-(5- | ¹H-NMR (400 MHz, DMSO-d₆) δ 8.89 (d, J = 1.2 Hz, 1H), |
| | fluoropyridin-3-yl)-2H- | 8.82 (d, J = 2.4 Hz, 1H), 8.33-8.29 (m, 1H), 7.58 (dd, J = 9.3, |
| | indazol-5- | 0.8 Hz, 1H), 7.05 (dd, J = 9.2, 1.8 Hz, 1H), 6.99 (q, J = 0.9 Hz, |
| | yl)imino)(methyl)(propyl)- | 1H), 3.37 (dd, J = 9.6, 6.0 Hz, 2H), 3.17 (s, 3H), 1.79 (dd, J = |
| | λ⁶-sulfanone | 16.7, 7.5 Hz, 2H), 0.98 (t, J = 7.3 Hz, 3H); ESI |
| | | MS(m/z): 366.95[M + 1]. |
| 201 | ((3-chloro-2- | ¹H-NMR (400 MHz, DMSO-d₆) δ 9.37 (s, 1H), 9.29 (s, 2H), |
| | (pyrimidin-5-yl)-2H- | 7.59 (dd, J = 9.2, 0.6 Hz, 1H), 7.09 (dd, J = 9.2, 1.8 Hz, 1H), |
| | indazol-5- | 7.00 (d, J = 1.2 Hz, 1H), 3.36 (t, J = 7.3 Hz, 4H), 1.25 (q, J = |
| | yl)imino)diethyl-λ⁶- | 7.4 Hz, 6H); ESI MS(m/z): 349.9[M + 1]. |
| | sulfanone | |
| 202 | ((7-chloro-2-(5- | ¹H-NMR (400 MHz, DMSO-d₆) δ 9.25 (s, 1H), 9.18 (s, 1H), |
| | fluoropyridin-3-yl)-2H- | 8.68 (d, J = 2.4 Hz, 1H), 8.52 (dt, J = 10.0, 2.2 Hz, 1H), 7.25 |
| | indazol-4- | (d, J = 7.8 Hz, 1H), 6.56 (d, J = 7.8 Hz, 1H), 3.34 (s, 6H) |
| | yl)imino)dimethyl-λ⁶- | ESI MS(m/z): 338.85[M + 1]. |
| | sulfanone | |
| 203 | ((3-chloro-2-(pyridazin- | ¹H-NMR (400 MHz, DMSO-d₆) δ 9.79 (s, 1H), 9.49 (d, J = |
| | 4-yl)-2H-indazol-5- | 5.2 Hz, 1H), 8.23 (s, 1H), 7.61 (d, J = 9.2 Hz, 1H), 7.07 (d, J = |
| | yl)imino)(methyl)(propyl)- | 8.9 Hz, 1H), 6.97 (s, 1H), 3.38 (t, J = 7.5 Hz, 2H), 3.18 (s, |
| | λ⁶-sulfanone | 3H), 1.79 (d, J = 7.0 Hz, 2H), 0.98 (t, J = 7.2 Hz, 3H) |
| | | ESI MS(m/z): 349.95[M + 1]. |
| 204 | ((3-chloro-2-(pyridazin- | ¹H-NMR (400 MHz, DMSO-d₆) δ 9.79 (q, J = 1.2 Hz, 1H), |
| | 4-yl)-2H-indazol-5- | 9.49 (dd, J = 5.7, 1.1 Hz, 1H), 8.23 (q, J = 2.8 Hz, 1H), 7.60 |
| | yl)imino)diethyl-λ⁶- | (dd, J = 9.3, 0.8 Hz, 1H), 7.10 (dd, J = 9.3, 2.0 Hz, 1H), 6.98 |
| | sulfanone | (q, J = 0.9 Hz, 1H), 3.36 (q, J = 7.4 Hz, 4H), 1.26 (t, J = 7.5 |
| | | Hz, 6H); ESI MS(m/z): 350[M + 1]. |
| 205 | ((3-chloro-2-(pyridazin- | ¹H-NMR (400 MHz, DMSO-d₆) δ 9.79 (q, J = 1.2 Hz, 1H), |
| | 4-yl)-2H-indazol-5- | 9.49 (dd, J = 5.8, 0.9 Hz, 1H), 8.23 (q, J = 2.8 Hz, 1H), 7.59 |
| | yl)imino)(ethyl)(isopropyl)- | (dd, J = 9.3, 0.8 Hz, 1H), 7.14-7.10 (m, 1H), 7.00-6.98 (m, |
| | λ⁶-sulfanone | 1H), 3.58 (td, J = 13.7, 6.8 Hz, 1H), 3.35 (d, J = 7.3 Hz, 2H), |
| | | 1.41-1.37 (m, 3H), 1.34-1.30 (m, 3H), 1.29-1.20 (m, 3H) |
| | | ESI MS(m/z): 363.9[M + 1]. |
| 206 | ((7-chloro-2-(5- | ¹H-NMR (400 MHz, DMSO-d₆) δ 9.27 (s, 1H), 9.20 (s, 1H), |
| | fluoropyridin-3-yl)-2H- | 8.68 (d, J = 2.8 Hz, 1H), 8.55 (dt, J = 10.0, 2.4 Hz, 1H), 7.23 |
| | indazol-4- | (d, J = 7.9 Hz, 1H), 6.59 (d, J = 7.9 Hz, 1H), 3.60 (t, J = 6.9 |
| | yl)imino)(isopropyl)(methyl)- | Hz, 1H), 3.17 (s, 3H), 1.48 (d, J = 6.7 Hz, 3H), 1.42 (d, J = 7.0 |
| | λ⁶-sulfanone | Hz, 3H); ESI MS(m/z): 366.9[M + 1]. |
| 207 | diethyl((2-(2- | ¹H-NMR (400 MHz, DMSO-d₆) δ 9.34 (d, J = 7.9 Hz, 2H), |
| | methylpyrimidin-5-yl)- | 8.93 (d, J = 0.9 Hz, 1H), 7.57 (d, J = 9.2 Hz, 1H), 7.17 (d, J = |
| | 2H-indazol-5- | 1.2 Hz, 1H), 7.01 (dd, J = 9.2, 2.1 Hz, 1H), 3.35-3.32 (m, 2H), |
| | yl)imino)-λ⁶-sulfanone | 3.30-3.27 (m, 2H), 2.70 (s, 3H), 1.31-1.17 (m, 6H) |
| | | ESI MS(m/z): 329.95[M + 1]. |
| 208 | cyclohexyl(methyl)((2- | ¹H-NMR (400 MHz, DMSO-d₆) δ 9.35 (d, J = 14.2 Hz, 2H), |
| | (2-methylpyrimidin-5- | 8.93 (d, J = 1.0 Hz, 1H), 7.58-7.55 (m, 1H), 7.17 (q, J = 1.0 |
| | yl)-2H-indazol-5- | Hz, 1H), 7.01 (dd, J = 9.2, 2.1 Hz, 1H), 3.03 (s, 3H), 2.76 (d, |
| | yl)imino)-λ⁶-sulfanone | J = 7.6 Hz, 1H), 2.70 (s, 3H), 2.10-2.04 (m, 1H), 1.64 (d, J = |
| | | 12.2 Hz, 2H), 1.58-1.39 (m, 2H), 1.36-1.09 (m, 6H) |
| | | ESI MS(m/z): 370.0[M + 1]. |
| 209 | isopropyl(methyl)((2- | ¹H-NMR (400 MHz, DMSO-d₆) δ 9.29 (d, J = 2.2 Hz, 1H), |
| | (pyridin-3-yl)-2H- | 9.06 (s, 1H), 8.62 (dd, J = 4.6, 1.5 Hz, 1H), 8.43 (dq, J = 8.3, |
| | indazol-7-yl)imino)-λ⁶- | 1.4 Hz, 1H), 7.63 (ddd, J = 8.3, 4.6, 0.7 Hz, 1H), 7.24 (dd, J = |
| | sulfanone | 8.3, 0.7 Hz, 1H), 6.92 (dd, J = 8.3, 7.1 Hz, 1H), 6.83 (dd, J = |
| | | 7.2, 0.9 Hz, 1H), 3.16 (d, J = 4.9 Hz, 4H), 1.46 (d, J = 6.8 Hz, |
| | | 3H), 1.40 (d, J = 6.8 Hz, 3H); ESI MS(m/z): 315.0[M + 1]. |
| 210 | diisobutyl((2-(2- | ¹H-NMR (400 MHz, DMSO-d₆) δ 9.33 (s, 2H), 8.93 (d, J = |
| | methylpyrimidin-5-yl)- | 1.0 Hz, 1H), 7.56 (d, J = 9.3 Hz, 1H), 7.16 (q, J = 0.9 Hz, 1H), |
| | 2H-indazol-5- | 7.00 (dd, J = 9.3, 2.0 Hz, 1H), 3.28-3.15 (m, 4H), 2.70 (s, 3H), |
| | yl)imino)-λ⁶-sulfanone | 2.32-2.24 (m, 2H), 1.03 (dd, J = 8.4, 6.7 Hz, 12H) |
| | | ESI MS(m/z): 386.2[M + 1]. |
| 211 | (2- | ¹H-NMR (400 MHz, DMSO-d₆) δ 9.34 (s, 2H), 8.94 (d, J = |
| | methoxyethyl)(methyl)((2- | 0.7 Hz, 1H), 7.59 (dt, J = 9.2, 0.9 Hz, 1H), 7.16 (q, J = 0.9 Hz, |
| | (2-methylpyrimidin- | 1H), 7.00 (dd, J = 9.3, 2.0 Hz, 1H), 3.74 (t, J = 5.6 Hz, 2H), |
| | 5-yl)-2H-indazol-5- | 3.61 (t, J = 5.4 Hz, 2H), 3.28 (s, 3H), 3.17 (s, 3H), 2.70 (s, 3H) |
| | yl)imino)-λ⁶-sulfanone | ESI MS(m/z): 346.05[M + 1]. |
| 212 | 3-(5-(((2- | ¹H-NMR (400 MHz, DMSO-d₆) δ 9.26 (s, 1H), 8.87 (d, J = |
| | methoxyethyl)(methyl)(oxo)- | 5.1 Hz, 1H), 8.84 (d, J = 0.7 Hz, 1H), 8.12 (dd, J = 5.0, 0.6 Hz, |
| | λ⁶- | 1H), 7.61 (d, J = 9.3 Hz, 1H), 7.21 (d, J = 1.2 Hz, 1H), 7.03 |
| | sulfaneylidene)amino)- | (dd, J = 9.3, 2.0 Hz, 1H), 3.74 (t, J = 5.5 Hz, 2H), 3.64 (d, J = |
| | 2H-indazol-2- | 5.4 Hz, 2H), 3.29 (s, 3H), 3.18 (s, 3H); ESI MS(m/z): |
| | yl)isonicotinonitrile | 356.0[M + 1]. |
| 213 | isopropyl(methyl)((2- | ¹H-NMR (400 MHz, DMSO-d₆) δ 9.47 (s, 2H), 9.21 (s, 1H), |
| | (pyrimidin-5-yl)-2H- | 9.08 (d, J = 0.6 Hz, 1H), 7.96 (dd, J = 9.2, 0.9 Hz, 1H), 6.81 |
| | pyrazolo[4,3-b]pyridin- | (d, J = 9.2 Hz, 1H), 3.82 (t, J = 6.9 Hz, 1H), 3.36 (s, 3H), 1.39 |
| | 5-yl)imino)-λ⁶- | (dd, J = 11.9, 7.0 Hz, 6H); ESI MS (m/z): 317.00[M + 1]. |
| | sulfanone | |
| 214 | butyl(ethyl)((2- | ¹H-NMR (400 MHz, DMSO-d₆) δ 9.27 (d, J = 2.4 Hz, 1H), |
| | (pyridin-3-yl)-2H- | 9.01 (d, J = 0.6 Hz, 1H), 8.60 (dd, J = 4.7, 1.4 Hz, 1H), 8.41 |
| | pyrazolo[4,3-b]pyridin- | (dq, J = 8.5, 1.4 Hz, 1H), 7.94 (dd, J = 9.2, 0.6 Hz, 1H), 7.62- |
| | 5-yl)imino)-λ⁶- | 7.58 (m, 1H), 6.79 (d, J = 9.2 Hz, 1H), 3.62-3.53 (m, 4H), |
| | sulfanone | 1.76-1.66 (m, 2H), 1.41 (q, J = 7.2 Hz, 2H), 1.28 (t, J = 7.3 |
| | | Hz, 3H), 0.89 (t, J = 7.3 Hz, 3H); ESI MS (m/z): 344.05[M + 1]. |
| 215 | diisobutyl((2-(pyridin- | ¹H-NMR (400 MHz, DMSO-d₆) δ 9.28 (d, J = 2.4 Hz, 1H), |
| | 3-yl)-2H-pyrazolo[4,3- | 9.02 (d, J = 0.9 Hz, 1H), 8.59 (dd, J = 4.7, 1.4 Hz, 1H), 8.42 |
| | b]pyridin-5-yl)imino)- | (dq, J = 8.3, 1.4 Hz, 1H), 7.94 (dd, J = 9.2, 0.9 Hz, 1H), 7.61- |
| | λ⁶-sulfanone | 7.58 (m, 1H), 6.79 (d, J = 9.5 Hz, 1H), 3.60 (dd, J = 13.9, 6.3 |
| | | Hz, 2H), 3.42 (dd, J = 14.1, 6.4 Hz, 2H), 2.34-2.27 (m, 2H), |
| | | 1.05-0.97 (m, 12H); ESI MS (m/z): 356.10[M + 1]. |
| 216 | cyclohexyl(methyl)((2- | ¹H-NMR (400 MHz, DMSO-d₆) δ 9.27 (d, J = 2.4 Hz, 1H), |
| | (pyridin-3-yl)-2H- | 9.03 (d, J = 0.6 Hz, 1H), 8.60 (dd, J = 4.7, 1.4 Hz, 1H), 8.42 |
| | pyrazolo[4,3-b]pyridin- | (dq, J = 8.3, 1.4 Hz, 1H), 7.94 (dd, J = 9.3, 0.8 Hz, 1H), 7.62- |
| | 5-yl)imino)-λ⁶- | 7.59 (m, 1H), 6.78 (d, J = 9.5 Hz, 1H), 3.57-3.51 (m, 1H), 3.36 |
| | sulfanone | (s, 3H), 2.23 (d, J = 12.2 Hz, 1H), 2.14 (d, J = 12.5 Hz, 1H), |
| | | 1.87 (d, J = 7.9 Hz, 2H), 1.67 (d, J = 12.5 Hz, 1H), 1.58-1.47 |
| | | (m, 2H), 1.35-1.27 (m, 2H), 1.24-1.16 (m, 1H); ESI MS |
| | | (m/z): 356.10 [M + 1]. |
| 217 | ((7-chloro-2-(pyridin-3- | ¹H-NMR (400 MHz, DMSO-d₆) δ 9.32 (d, J = 2.4 Hz, 1H), |
| | yl)-2H-indazol-4- | 9.11 (s, 1H), 8.64 (dd, J = 4.6, 1.2 Hz, 1H), 8.50 (dq, J = 8.3, |
| | yl)imino)dimethyl-λ⁶- | 1.4 Hz, 1H), 7.64-7.60 (m, 1H), 7.24 (d, J = 7.8 Hz, 1H), 6.56 |
| | sulfanone | (d, J = 7.8 Hz, 1H), 3.33 (s, 6H); ESI MS(m/z): 320.85[M + 1]. |
| 218 | ((7-chloro-2-(pyridin-3- | ¹H-NMR (400 MHz, DMSO-d₆) δ 9.34 (d, J = 2.4 Hz, 1H), |
| | yl)-2H-indazol-4- | 9.12 (d, J = 11.7 Hz, 1H), 8.65 (dd, J = 4.6, 1.5 Hz, 1H), 8.51 |
| | yl)imino)(isopropyl)(methyl)- | (dq, J = 8.3, 1.4 Hz, 1H), 7.64-7.61 (m, 1H), 7.22 (d, J = 7.8 |
| | λ⁶-sulfanone | Hz, 1H), 6.59 (d, J = 7.6 Hz, 1H), 3.63-3.56 (m, 1H), 3.16 (s, |
| | | 3H), 1.47 (d, J = 6.8 Hz, 3H), 1.41 (d, J = 6.6 Hz, 3H); ESI |
| | | MS (m/z): 348.9 [M + 1]. |
| 219 | diethyl((2-(pyrimidin-5- | ¹H-NMR (400 MHz, DMSO-d₆) δ 9.46 (s, 2H), 9.21 (s, 1H), |
| | yl)-2H-pyrazolo[4,3- | 9.07 (d, J = 1.0 Hz, 1H), 7.96 (dd, J = 9.3, 1.0 Hz, 1H), 6.82 |
| | b]pyridin-5-yl)imino)- | (d, J = 9.3 Hz, 1H), 3.58 (q, J = 7.3 Hz, 4H), 1.28 (t, J = 7.5 |
| | λ⁶-sulfanone | Hz, 6H); ESI MS (m/z): 317.00[M + 1]. |
| 220 | butyl(ethyl)((2- | ¹H-NMR (400 MHz, DMSO-d₆) δ 9.47 (s, 2H), 9.21 (s, 1H), |
| | (pyrimidin-5-yl)-2H- | 9.07 (d, J = 0.9 Hz, 1H), 7.96 (dd, J = 9.2, 0.9 Hz, 1H), 6.81 |
| | pyrazolo[4,3-b]pyridin- | (d, J = 9.2 Hz, 1H), 3.62-3.54 (m, 4H), 1.78-1.65 (m, 2H), |
| | 5-yl)imino)-λ⁶- | 1.45-1.38 (m, 2H), 1.28 (t, J = 7.3 Hz, 3H), 0.89 (t, J = 7.3 Hz, |
| | sulfanone | 3H); ESI MS (m/z): 345.10[M + 1]. |
| 221 | diisobutyl((2- | ¹H-NMR (400 MHz, DMSO-d₆) δ 9.47 (s, 2H), 9.20 (s, 1H), |
| | (pyrimidin-5-yl)-2H- | 9.07 (d, J = 0.7 Hz, 1H), 7.96 (dd, J = 9.2, 0.9 Hz, 1H), 6.81 |
| | pyrazolo[4,3-b]pyridin- | (d, J = 9.3 Hz, 1H), 3.61 (dd, J = 13.9, 6.4 Hz, 2H), 3.42 (dd, |
| | 5-yl)imino)-λ⁶- | J = 13.9, 6.4 Hz, 2H), 2.36-2.26 (m, 2H), 1.08-1.02 (m, 12H) |
| | sulfanone | ESI MS (m/z): 373.15[M + 1]. |
| 222 | methyl(propyl)((2- | ¹H-NMR (400 MHz, DMSO-d₆) δ 9.43-9.51 (2H), 9.18-9.24 |
| | (pyrimidin-5-yl)-2H- | (1H), 9.05-9.12 (1H), 7.92-8.01 (1H), 6.75-6.85 (1H), 3.48- |
| | pyrazolo[4,3-b]pyridin- | 3.66 (2H), 3.35-3.43 (3H), 1.66-1.91 (2H), 0.93-1.07 (3H) |
| | 5-ylimino)-λ⁶- | ESI MS (m/z): 317.00[M + 1]. |
| | sulfanone | |
| 223 | cyclohexyl(methyl)((2- | ¹H-NMR (400 MHz, DMSO-d₆) δ 9.47 (s, 2H), 9.21 (s, 1H), |
| | (pyrimidin-5-yl)-2H- | 9.09 (d, J = 0.6 Hz, 1H), 7.96 (dd, J = 9.3, 0.8 Hz, 1H), 6.80 |
| | pyrazolo[4,3-b]pyridin- | (d, J = 9.2 Hz, 1H), 3.58-3.52 (m, 1H), 3.36 (s, 3H), 2.18 (dd, |
| | 5-yl)imino)-λ⁶- | J = 34.4, 12.4 Hz, 2H), 1.86 (s, 2H), 1.67 (d, J = 11.9 Hz, 1H), |
| | sulfanone | 1.56-1.48 (m, 2H), 1.34-1.16 (m, 3H); ESI MS (m/z): 357.10 |
| | | [M + 1]. |
| 224 | dimethyl((2-(pyridin-3- | ¹H-NMR (400 MHz, DMSO-d₆) δ 9.27 (d, J = 2.4 Hz, 1H), |
| | yl)-2H-pyrazolo[4,3- | 9.03 (d, J = 0.7 Hz, 1H), 8.60 (dd, J = 4.6, 1.5 Hz, 1H), 8.41 |
| | b]pyridin-5-yl)imino)- | (dq, J = 8.3, 1.4 Hz, 1H), 7.96 (dd, J = 9.3, 1.0 Hz, 1H), 7.61 |
| | λ⁶-sulfanone | (ddd, J = 8.4, 4.7, 0.6 Hz, 1H), 6.76 (d, J = 9.3 Hz, 1H), 3.43 |
| | | (s, 6H); ESI MS (m/z): 287.95[M + 1]. |
| 229 | dimethyl((2-(pyridin-3- | ¹H-NMR (400 MHz, DMSO-d₆) δ 9.29 (d, J = 2.2 Hz, 1H), |
| | yl)-6-(trifluoromethyl)- | 9.08 (d, J = 0.7 Hz, 1H), 8.66 (dd, J = 4.8, 1.3 Hz, 1H), 8.44 |
| | 2H-indazol-5- | (dq, J = 8.3, 1.4 Hz, 1H), 8.08 (s, 1H), 7.65 (ddd, J = 8.3, 4.8, |
| | yl)imino)-λ⁶-sulfanone | 0.6 Hz, 1H), 7.46 (s, 1H), 3.22 (s, 6H); ESI MS(m/z): 355.0 |
| | | [M + 1]. |
| 232 | dimethyl((2-(pyridin-3- | ¹H-NMR (400 MHz, CHLOROFORM-D) δ 9.19 (d, J = 2.8 |
| | yl)-2H-pyrazolo[3,4- | Hz, 1H), 9.17 (s, 1H), 8.71 (dd, J = 4.7, 1.4 Hz, 1H), 8.34 (d, |
| | c]pyridin-5-yl)imino)- | J = 0.6 Hz, 1H), 8.29 (dq, J = 8.3, 1.3 Hz, 1H), 7.52 (q, J = 4.2 |
| | λ⁶-sulfanone | Hz, 1H), 7.01 (d, J = 1.2 Hz, 1H), 3.40 (s, 6H) |
| | | ESI MS (m/z): 288.0[M + 1]. |
| 233 | diisobutyl((2-(pyridin- | ¹H-NMR (400 MHz, DMSO-d₆) δ 9.28 (d, J = 2.4 Hz, 1H), |
| | 3-yl)-2H-indazol-7- | 9.04 (s, 1H), 8.62 (dd, J = 4.6, 1.2 Hz, 1H), 8.43-8.40 (m, 1H), |
| | yl)imino)-λ⁶-sulfanone | 7.63 (dd, J = 8.4, 4.8 Hz, 1H), 7.22 (d, J = 8.3 Hz, 1H), 6.93- |
| | | 6.90 (m, 1H), 6.81 (d, J = 7.1 Hz, 1H), 3.39 (dd, J = 14.2, 6.1 |
| | | Hz, 2H), 3.30-3.26 (m, 2H), 2.35 (d, J = 6.6 Hz, 2H), 1.05 (q, |
| | | J = 6.5 Hz, 12H); ESI MS(m/z): 371.10[M + 1]. |
| 234 | (2- | ¹H-NMR (400 MHz, CHLOROFORM-D) δ 9.17 (d, J = 2.2 |
| | methoxyethyl)(methyl)((2- | Hz, 1H), 8.68 (d, J = 3.7 Hz, 1H), 8.32 (d, J = 1.0 Hz, 1H), |
| | (pyridin-3-yl)-6- | 8.25 (dq, J = 8.3, 1.3 Hz, 1H), 8.14 (s, 1H), 7.60 (s, 1H), 7.50 |
| | (trifluoromethyl)-2H- | (q, J = 4.2 Hz, 1H), 3.97-3.92 (m, 1H), 3.88-3.81 (m, 1H), |
| | indazol-5-yl)imino)-λ⁶- | 3.45 (t, J = 5.0 Hz, 2H), 3.41-3.39 (m, 3H), 3.15 (s, 3H) |
| | sulfanone | ESI MS(m/z): 399.10[M + 1]. |
| 235 | isopropyl(methyl)((2- | ¹H-NMR (400 MHz, CHLOROFORM-D) δ 9.19 (d, J = 2.4 |
| | (pyridin-3-yl)-2H- | Hz, 1H), 9.15 (s, 1H), 8.70 (dd, J = 4.7, 1.4 Hz, 1H), 8.31 (d, J = |
| | pyrazolo[3,4-c]pyridin- | 0.6 Hz, 1H), 8.28 (dq, J = 8.3, 1.3 Hz, 1H), 7.51 (dd, J = 8.3, |
| | 5-yl)imino)-λ⁶- | 4.9 Hz, 1H), 7.02 (d, J = 0.9 Hz, 1H), 3.80-3.73 (m, 1H), 3.22 |
| | sulfanone | (s, 3H), 1.57 (dd, J = 12.4, 5.3 Hz, 3H), 1.52 (d, J = 7.0 Hz, |
| | | 3H); ESI MS (m/z): 316.1[M + 1]. |
| 236 | (2- | ¹H-NMR (400 MHz, DMSO-d₆) δ 9.27 (d, J = 2.4 Hz, 1H), |
| | methoxyethyl)(methyl)((2- | 9.04 (d, J = 0.6 Hz, 1H), 8.60 (dd, J = 4.6, 1.2 Hz, 1H), 8.41 |
| | (pyridin-3-yl)-2H- | (dq, J = 8.4, 1.3 Hz, 1H), 7.97 (dd, J = 9.2, 0.6 Hz, 1H), 7.60 |
| | pyrazolo[4,3-b]pyridin- | (dd, J = 8.6, 4.9 Hz, 1H), 6.79 (d, J = 9.2 Hz, 1H), 3.97-3.85 |
| | 5-yl)imino)-λ⁶- | (m, 2H), 3.83-3.75 (m, 2H), 3.37 (s, 3H), 3.31 (s, 3H) |
| | sulfanone | ESI MS (m/z): 332.00[M + 1]. |
| 237 | (2- | ¹H-NMR (400 MHz, DMSO-d₆) δ 9.47 (s, 2H), 9.21 (s, 1H), |
| | methoxyethyl)(methyl)((2- | 9.09 (d, J = 0.9 Hz, 1H), 7.99 (dd, J = 9.2, 0.6 Hz, 1H), 6.82 |
| | (pyrimidin-5-yl)-2H- | (d, J = 9.2 Hz, 1H), 3.98-3.85 (m, 2H), 3.84-3.77 (m, 2H), |
| | pyrazolo[4,3-b]pyridin- | 3.37 (s, 3H), 3.31 (s, 3H); ESI MS (m/z): 333.00[M + 1]. |
| | 5-yl)imino)-λ⁶- | |
| | sulfanone | |
| 238 | isopropyl(methyl)((2- | ¹H-NMR (400 MHz, DMSO-d₆) δ 9.27 (d, J = 2.4 Hz, 1H), |
| | (pyridin-3-yl)-2H- | 9.03 (s, 1H), 8.60 (dd, J = 4.6, 1.2 Hz, 1H), 8.41 (dq, J = 8.4, |
| | pyrazolo[4,3-b]pyridin- | 1.3 Hz, 1H), 7.95 (d, J = 9.2 Hz, 1H), 7.60 (dd, J = 8.4, 4.7 Hz, |
| | 5-yl)imino)-λ⁶- | 1H), 6.79 (d, J = 9.2 Hz, 1H), 3.86-3.79 (m, 1H), 3.37 (d, J = |
| | sulfanone | 8.9 Hz, 3H), 1.41-1.31 (m, 6H); ESI MS (m/z): 316.00[M + 1]. |
| 239 | methyl(propyl)((2- | ¹H-NMR (400 MHz, DMSO-d₆) δ 9.27 (d, J = 2.8 Hz, 1H), |
| | (pyridin-3-yl)-2H- | 9.03 (d, J = 0.6 Hz, 1H), 8.60 (dd, J = 4.6, 1.2 Hz, 1H), 8.42 |
| | pyrazolo[4,3-b]pyridin- | (dq, J = 8.3, 1.4 Hz, 1H), 7.95 (dd, J = 9.2, 0.6 Hz, 1H), 7.60 |
| | 5-yl)imino)-λ⁶- | (q, J = 4.3 Hz, 1H), 6.77 (d, J = 9.5 Hz, 1H), 3.59-3.54 (m, |
| | sulfanone | 2H), 3.38 (s, 3H), 1.83-1.76 (m, 2H), 1.00 (t, J = 7.5 Hz, 3H); |
| | | ESI MS (m/z): 316.05[M + 1]. |
| 240 | ethyl(isopropyl)((2- | ¹H-NMR (400 MHz, DMSO-d₆) δ 9.27 (d, J = 2.4 Hz, 1H), |
| | (pyridin-3-yl)-2H- | 9.01 (s, 1H), 8.59 (dd, J = 4.6, 1.2 Hz, 1H), 8.41 (dq, J = 8.3, |
| | pyrazolo[4,3-b]pyridin- | 1.3 Hz, 1H), 7.94 (d, J = 9.2 Hz, 1H), 7.60 (dd, J = 8.3, 4.9 Hz, |
| | 5-yl)imino)-λ⁶- | 1H), 6.81 (d, J = 9.5 Hz, 1H), 3.94-3.87 (m, 1H), 3.69-3.59 |
| | sulfanone | (m, 2H), 1.37 (dd, J = 9.2, 7.0 Hz, 6H), 1.28 (t, J = 7.3 Hz, |
| | | 3H); ESI MS (m/z): 330.05[M + 1]. |
| 241 | ethyl(isopropyl)((2- | ¹H-NMR (400 MHz, DMSO-d₆) δ 9.46 (s, 2H), 9.21 (s, 1H), |
| | (pyrimidin-5-yl)-2H- | 9.06 (d, J = 0.9 Hz, 1H), 7.96 (dd, J = 9.2, 0.9 Hz, 1H), 6.83 |
| | pyrazolo[4,3-b]pyridin- | (d, J = 9.2 Hz, 1H), 3.90 (t, J = 6.9 Hz, 1H), 3.64 (qd, J = 7.2, |
| | 5-yl)imino)-λ⁶- | 4.5 Hz, 2H), 1.37 (dd, J = 8.6, 6.7 Hz, 6H), 1.28 (t, J = 7.3 Hz, |
| | sulfanone | 3H); ESI MS (m/z): 331.00[M + 1]. |
| 242 | dimethyl((2-(pyrimidin- | ¹H-NMR (400 MHz, DMSO-d₆) δ 9.54 (s, 2H), 9.29 (s, 1H), |
| | 5-yl)-2H-pyrazolo[3,4- | 9.10 (t, J = 1.1 Hz, 1H), 9.08 (d, J = 0.7 Hz, 1H), 6.81 (d, J = |
| | c]pyridin-5-yl)imino)- | 1.2 Hz, 1H), 3.36 (s, 6H); ESI MS (m/z): 288.9[M + 1]. |
| | λ⁶-sulfanone | |
| 243 | ((2-(5-fluoropyridin-3- | ¹H-NMR (400 MHz, DMSO-d₆) δ 9.25 (s, 1H), 9.07 (dd, J = |
| | yl)-2H-pyrazolo[3,4- | 10.0, 1.0 Hz, 2H), 8.72 (d, J = 2.4 Hz, 1H), 8.51 (dt, J = 9.9, |
| | c]pyridin-5- | 2.3 Hz, 1H), 6.79 (d, J = 1.5 Hz, 1H), 3.36 (s, 6H) |
| | yl)imino)dimethyl-λ⁶- | ESI MS(m/z): 305.9[M + 1]. |
| | sulfanone | |
| 244 | ((2-(5-fluoropyridin-3- | ¹H-NMR (400 MHz, DMSO-d₆) δ 9.25 (s, 1H), 9.07 (dd, J = |
| | yl)-2H-pyrazolo[3,4- | 5.1, 1.0 Hz, 2H), 8.72 (d, J = 2.4 Hz, 1H), 8.53 (dt, J = 9.9, 2.3 |
| | c]pyridin-5- | Hz, 1H), 6.83 (d, J = 1.5 Hz, 1H), 3.70 (t, J = 6.8 Hz, 1H), |
| | yl)imino)(isopropyl)(methyl)- | 3.24 (s, 3H), 1.41 (d, J = 6.8 Hz, 3H), 1.36 (d, J = 6.8 Hz, 3H) |
| | λ⁶-sulfanone | ESI MS(m/z): 334.15[M + 1]. |
| 245 | diethyl((2-(5- | ¹H-NMR (400 MHz, DMSO-d₆) δ 9.25 (s, 1H), 9.06 (s, 2H), |
| | fluoropyridin-3-yl)-2H- | 8.72 (d, J = 2.7 Hz, 1H), 8.52 (dt, J = 9.9, 2.3 Hz, 1H), 6.83 (d, |
| | pyrazolo[3,4-c]pyridin- | J = 1.0 Hz, 1H), 3.52-3.41 (m, 4H), 1.29-1.22 (m, 6H) |
| | 5-ylimino)-λ⁶- | ESI MS(m/z): 334.15[M + 1]. |
| | sulfanone | |
| 246 | ((2-(5-fluoropyridin-3- | ¹H-NMR (400 MHz, DMSO-d₆)¹H-NMR (400 MHz, DMSO- |
| | yl)-2H-pyrazolo[3,4- | d₆) δ 9.25 (s, 1H), 9.08-9.06 (m, 2H), 8.72 (d, J = 2.4 Hz, 1H), |
| | c]pyridin-5- | 8.52 (dt, J = 9.9, 2.3 Hz, 1H), 6.80 (d, J = 1.5 Hz, 1H), 3.49 (t, |
| | yl)imino)(methyl)(propyl)- | J = 7.9 Hz, 2H), 3.31 (s, 3H), 1.84-1.73 (m, 2H), 0.99 (t, J = |
| | λ⁶-sulfanone | 7.5 Hz, 3H); ESI MS(m/z): 334.15[M + 1]. |
| 247 | 1-((2-(5-fluoropyridin- | ¹H-NMR (400 MHz, DMSO-d₆) δ 9.26 (s, 1H), 9.10-9.08 (m, |
| | 3-yl)-2H-pyrazolo[3,4- | 2H), 8.73 (d, J = 2.4 Hz, 1H), 8.53 (dt, J = 9.8, 2.3 Hz, 1H), |
| | c]pyridin-5- | 6.85 (d, J = 1.5 Hz, 1H), 3.56-3.49 (m, 2H), 3.35 (d, J = 6.7 |
| | yl)imino)tetrahydro- | Hz, 1H), 3.30 (d, J = 7.3 Hz, 1H), 2.24-2.06 (m, 4H) |
| | 1H-λ⁶-thiophene 1- | ESI MS(m/z): 331.9[M + 1]. |
| | oxide | |
| 248 | ethyl(methyl)((2- | ¹H-NMR (400 MHz, DMSO-d₆) δ 9.27 (d, J = 2.8 Hz, 1H), |
| | (pyridin-3-yl)-2H- | 9.03 (d, J = 0.6 Hz, 1H), 8.60 (dd, J = 4.7, 1.4 Hz, 1H), 8.41 |
| | pyrazolo[4,3-b]pyridin- | (dq, J = 8.3, 1.4 Hz, 1H), 7.95 (dd, J = 9.2, 0.6 Hz, 1H), 7.60 |
| | 5-yl)imino)-λ⁶- | (dd, J = 8.4, 4.7 Hz, 1H), 6.78 (d, J = 9.2 Hz, 1H), 3.60 (q, J = |
| | sulfanone | 7.4 Hz, 2H), 3.36 (s, 3H), 1.31 (t, J = 7.5 Hz, 3H) |
| | | ESI MS(m/z): 301.910[M + 1]. |
| 249 | diethyl((2-(pyridin-3- | ¹H-NMR (400 MHz, DMSO-d₆) δ 9.27 (d, J = 2.4 Hz, 1H), |
| | yl)-2H-pyrazolo[4,3- | 9.02 (d, J = 0.6 Hz, 1H), 8.60 (dd, J = 4.7, 1.4 Hz, 1H), 8.41 |
| | b]pyridin-5-yl)imino)- | (dq, J = 8.4, 1.3 Hz, 1H), 7.95 (dd, J = 9.3, 0.8 Hz, 1H), 7.60 |
| | λ⁶-sulfanone | (dd, J = 8.4, 4.7 Hz, 1H), 6.79 (d, J = 9.2 Hz, 1H), 3.57 (q, J = |
| | | 7.4 Hz, 4H), 1.28 (t, J = 7.3 Hz, 6H); ESI MS(m/z): 315.9 |
| | | [M + 1]. |
| 250 | 1-((2-(pyridin-3-yl)-2H- | ¹H-NMR (400 MHz, DMSO-d₆) δ 9.28 (d, J = 2.4 Hz, 1H), |
| | pyrazolo[4,3-b]pyridin- | 9.06 (d, J = 0.6 Hz, 1H), 8.60 (dd, J = 4.7, 1.4 Hz, 1H), 8.43 |
| | 5-yl)imino)tetrahydro- | (dq, J = 8.5, 1.4 Hz, 1H), 7.98 (dd, J = 9.2, 0.6 Hz, 1H), 7.61 |
| | 1H-λ⁶-thiophene 1- | (q, J = 4.3 Hz, 1H), 6.83 (d, J = 9.2 Hz, 1H), 3.67-3.60 (m, |
| | oxide | 2H), 3.35 (q, J = 6.7 Hz, 2H), 2.24-2.19 (m, 2H), 2.15-2.10 |
| | | (m, 2H); ESI MS(m/z): 313.9[M + 1]. |
| 251 | isopropyl(methyl)((2- | ¹H-NMR (400 MHz, DMSO-d₆) δ 9.54 (s, 2H), 9.29 (s, 1H), |
| | (pyrimidin-5-yl)-2H- | 9.10-9.09 (m, 2H), 6.84 (d, J = 1.5 Hz, 1H), 3.74-3.67 (m, |
| | pyrazolo[3,4-c]pyridin- | 1H), 3.25 (s, 3H), 1.41 (d, J = 7.0 Hz, 3H), 1.36 (d, J = 6.7 Hz, |
| | 5-yl)imino)-λ⁶- | 3H); ESI MS(m/z): 317[M + 1]. |
| | sulfanone | |
| 252 | methyl(propyl)((2- | ¹H-NMR (400 MHz, DMSO-d₆) δ 9.54 (d, J = 3.1 Hz, 2H), |
| | (pyrimidin-5-yl)-2H- | 9.30 (d, J = 10.7 Hz, 1H), 9.09 (dd, J = 5.0, 1.1 Hz, 2H), 6.82 |
| | pyrazolo[3,4-c]pyridin- | (d, J = 1.5 Hz, 1H), 3.51-3.46 (m, 2H), 3.29 (s, 3H), 1.84-1.73 |
| | 5-ylimino)-λ⁶- | (m, 2H), 1.01-0.96 (m, 3H); ESI MS(m/z): 317.05[M + 1]. |
| | sulfanone | |
| 253 | dimethyl((2-(pyrimidin- | ¹H-NMR (400 MHz, DMSO-d₆) δ 9.50 (s, 2H), 9.28 (s, 1H), |
| | 5-yl)-6- | 9.14 (d, J = 0.9 Hz, 1H), 8.11 (s, 1H), 7.46 (s, 1H), 3.24 (d, J = |
| | (trifluoromethyl)-2H- | 4.9 Hz, 6H); ESI MS(m/z): 355.85[M + 1]. |
| | indazol-5-yl)imino)-λ⁶- | |
| | sulfanone | |
| 254 | isopropyl(propyl)((2- | ¹H-NMR (400 MHz, DMSO-d₆) δ 9.27 (d, J = 2.4 Hz, 1H), |
| | (pyridin-3-yl)-2H- | 9.02 (d, J = 0.9 Hz, 1H), 8.59 (dd, J = 4.6, 1.2 Hz, 1H), 8.41 |
| | pyrazolo[4,3-b]pyridin- | (dq, J = 8.3, 1.4 Hz, 1H), 7.94 (dd, J = 9.2, 0.6 Hz, 1H), 7.60 |
| | 5-yl)imino)-λ⁶- | (dd, J = 8.4, 4.7 Hz, 1H), 6.80 (d, J = 9.2 Hz, 1H), 3.97-3.90 |
| | sulfanone | (m, 1H), 3.63-3.49 (m, 2H), 1.85-1.69 (m, 2H), 1.37 (dd, J = |
| | | 8.9, 7.0 Hz, 6H), 0.99 (t, J = 7.3 Hz, 3H) |
| | | ESI MS(m/z): 344.05[M + 1]. |
| 255 | diisopropyl((2-(pyridin- | ¹H-NMR (400 MHz, DMSO-d₆) δ 9.27 (d, J = 2.4 Hz, 1H), |
| | 3-yl)-2H-pyrazolo[4,3- | 9.00 (d, J = 0.6 Hz, 1H), 8.59 (dd, J = 4.7, 1.4 Hz, 1H), 8.42 |
| | b]pyridin-5-yl)imino)- | (dq, J = 8.3, 1.4 Hz, 1H), 7.93 (dd, J = 9.2, 0.9 Hz, 1H), 7.60 |
| | λ⁶-sulfanone | (dd, J = 8.4, 4.7 Hz, 1H), 6.85 (d, J = 9.2 Hz, 1H), 3.97-3.91 |
| | | (m, 2H), 1.40 (q, J = 7.1 Hz, 12H); ESI MS(m/z): 344.05 |
| | | [M + 1]. |
| 256 | isopropyl(propyl)((2- | ¹H-NMR (400 MHz, DMSO-d₆) δ 9.26 (d, J = 2.4 Hz, 1H), |
| | (pyridin-3-yl)-2H- | 8.91 (d, J = 0.6 Hz, 1H), 8.60 (dd, J = 4.7, 1.4 Hz, 1H), 8.40 |
| | indazol-5-yl)imino)-λ⁶- | (dq, J = 8.4, 1.3 Hz, 1H), 7.62-7.59 (m, 1H), 7.55 (d, J = 9.2 |
| | sulfanone | Hz, 1H), 7.17 (t, J = 0.9 Hz, 1H), 7.02 (dd, J = 9.2, 2.1 Hz, |
| | | 1H), 3.56-3.49 (m, 1H), 3.28-3.21 (m, 2H), 1.78-1.69 (m, 2H), |
| | | 1.37 (d, J = 7.0 Hz, 3H), 1.30 (d, J = 7.0 Hz, 3H), 0.96 (td, J = |
| | | 7.3, 3.4 Hz, 3H); ESI MS(m/z): 343.1[M + 1]. |
| 257 | isopropyl(propyl)((2- | ¹H-NMR (400 MHz, DMSO-d₆) δ 9.47 (s, 2H), 9.21 (s, 1H), |
| | (pyrimidin-5-yl)-2H- | 8.97 (s, 1H), 7.57 (d, J = 9.2 Hz, 1H), 7.18 (d, J = 1.2 Hz, 1H), |
| | indazol-5-yl)imino)-λ⁶- | 7.04 (dd, J = 9.2, 2.1 Hz, 1H), 3.53 (t, J = 6.7 Hz, 1H), 3.25 |
| | sulfanone | (dd, J = 16.0, 7.5 Hz, 2H), 1.74 (q, J = 7.7 Hz, 2H), 1.37 (d, |
| | | J = 6.7 Hz, 3H), 1.30 (d, J = 6.7 Hz, 3H), 0.95 (t, J = 7.5 Hz, |
| | | 3H); ESI MS(m/z): 344.1[M + 1]. |
| 258 | ((2-(5-fluoropyridin-3- | ¹H-NMR (400 MHz, DMSO-d₆) δ 9.19 (s, 1H), 8.95 (d, J = |
| | yl)-2H-indazol-5- | 0.9 Hz, 1H), 8.63 (d, J = 2.4 Hz, 1H), 8.41 (dt, J = 10.2, 2.4 |
| | yl)imino)(isopropyl)(propyl)- | Hz, 1H), 7.55 (d, J = 9.2 Hz, 1H), 7.16 (t, J = 0.9 Hz, 1H), |
| | λ⁶-sulfanone | 7.03 (dd, J = 9.2, 2.1 Hz, 1H), 3.56-3.49 (m, 1H), 3.25-3.21 |
| | | (m, 2H), 1.79-1.69 (m, 2H), 1.36 (t, J = 6.7 Hz, 3H), 1.31 (t, |
| | | J = 6.4 Hz, 3H), 0.97-0.93 (m, 3H);ESI MS(m/z): 360.95[M + 1]. |
| 259 | ((4-chloro-2- | ¹H-NMR (400 MHz, DMSO-d₆) δ 9.54 (s, 2H), 9.24 (s, 1H), |
| | (pyrimidin-5-yl)-2H- | 9.14 (d, J = 0.6 Hz, 1H), 7.59 (dd, J = 9.2, 0.9 Hz, 1H), 7.38 |
| | indazol-5- | (d, J = 9.2 Hz, 1H), 3.34 (d, J = 7.3 Hz, 2H), 3.29 (d, J = 7.0 |
| | yl)imino)diethyl-λ⁶- | Hz, 2H), 1.29 (t, J = 7.3 Hz, 6H); ESI MS (m/z): 349.80[M + 1]. |
| | sulfanone | |
| 260 | ((4-chloro-2- | ¹H-NMR (400 MHz, DMSO-d₆) δ 9.53 (s, 2H), 9.23 (s, 1H), |
| | (pyrimidin-5-yl)-2H- | 9.13 (s, 1H), 7.57 (d, J = 8.3 Hz, 1H), 7.41 (d, J = 9.0 Hz, 1H), |
| | indazol-5- | 3.57-3.50 (m, 1H), 3.29-3.27 (m, 2H), 1.40 (d, J = 6.6 Hz, |
| | yl)imino)(ethyl)(isopropyl)- | 3H), 1.32 (d, J = 6.8 Hz, 3H), 1.26 (t, J = 7.5 Hz, 3H) |
| | λ⁶-sulfanone | ESI MS (m/z): 363.90[M + 1]. |
| 261 | ((4-chloro-2- | ¹H-NMR (400 MHz, DMSO-d₆) δ 9.54 (s, 2H), 9.24 (s, 1H), |
| | (pyrimidin-5-yl)-2H- | 9.15 (s, 1H), 7.60 (d, J = 9.0 Hz, 1H), 7.36 (d, J = 9.3 Hz, 1H), |
| | indazol-5- | 3.36 (dd, J = 9.7, 6.2 Hz, 2H), 3.16 (s, 3H), 1.87-1.79 (m, 2H), |
| | yl)imino)(methyl)(propyl)- | 0.99 (t, J = 7.5 Hz, 3H); ESI MS (m/z): 349.80[M + 1]. |
| | λ⁶-sulfanone | |
| 262 | ((4-chloro-2- | ¹H-NMR (400 MHz, DMSO-d₆) δ 9.54 (s, 2H), 9.24 (s, 1H), |
| | (pyrimidin-5-yl)-2H- | 9.17 (d, J = 0.7 Hz, 1H), 7.61 (dd, J = 9.2, 0.9 Hz, 1H), 7.36 |
| | indazol-5-yl)imino)(2- | (d, J = 9.0 Hz, 1H), 3.85-3.80 (m, 1H), 3.77-3.72 (m, 1H), |
| | methoxyethyl)(methyl)- | 3.69-3.56 (m, 2H), 3.29 (s, 3H), 3.19 (s, 3H); ESI MS |
| | λ⁶-sulfanone | (m/z): 366.00 [M + 1]. |
| 263 | ((4-chloro-2- | ¹H-NMR (400 MHz, DMSO-d₆) δ 9.54 (s, 2H), 9.24 (s, 1H), |
| | (pyrimidin-5-yl)-2H- | 9.16 (d, J = 0.9 Hz, 1H), 7.61 (dd, J = 8.9, 0.9 Hz, 1H), 7.34 |
| | indazol-5- | (d, J = 9.2 Hz, 1H), 3.25 (s, 6H); ESI MS (m/z): 321.80[M + 1]. |
| | yl)imino)dimethyl-λ⁶- | |
| | sulfanone | |
| 264 | diethyl((2-(pyridin-3- | ¹H-NMR (400 MHz, DMSO-d₆) d 9.28 (d, J = 2.4 Hz, 1H), |
| | yl)-6-(trifluoromethyl)- | 9.05 (s, 1H), 8.66 (dd, J = 4.8, 1.3 Hz, 1H), 8.45-8.42 (m, 1H), |
| | 2H-indazol-5- | 8.08 (s, 1H), 7.65 (dd, J = 8.4, 5.0 Hz, 1H), 7.45 (s, 1H), 3.35 |
| | yl)imino)-λ⁶-sulfanone | (q, J = 7.4 Hz, 4H), 1.26 (t, J = 7.5 Hz, 6H); ESI MS(m/z): |
| | | 382.85[M + 1]. |
| 265 | ethyl(isopropyl)((2- | ¹H-NMR (400 MHz, DMSO-d₆) δ 9.28 (d, J = 2.2 Hz, 1H), |
| | (pyridin-3-yl)-6- | 9.04 (d, J = 0.7 Hz, 1H), 8.66 (dd, J = 4.8, 1.3 Hz, 1H), 8.43 |
| | (trifluoromethyl)-2H- | (dq, J = 8.4, 1.3 Hz, 1H), 8.08 (s, 1H), 7.65 (q, J = 4.6 Hz, |
| | indazol-5-yl)imino)-λ⁶- | 1H), 7.46 (s, 1H), 3.57 (q, J = 6.8 Hz, 1H), 3.36 (dd, J = 15.4, |
| | sulfanone | 7.3 Hz, 2H), 1.36 (q, J = 6.7 Hz, 6H), 1.27-1.21 (m, 3H) |
| | | ESI MS(m/z): 397.05[M + 1]. |
| 266 | ethyl(isopropyl)((2- | ¹H-NMR (400 MHz, DMSO-d₆) δ 9.49 (s, 2H), 9.27 (s, 1H), |
| | (pyrimidin-5-yl)-6- | 9.10 (s, 1H), 8.10 (s, 1H), 7.46 (s, 1H), 3.62-3.55 (m, 1H), |
| | (trifluoromethyl)-2H- | 3.37 (q, J = 7.5 Hz, 2H), 1.36 (q, J = 6.7 Hz, 6H), 1.27-1.22 |
| | indazol-5-yl)imino)-λ⁶- | (m, 4H); ESI MS(m/z): 397.95[M + 1]. |
| | sulfanone | |
| 267 | (3- | ¹H-NMR (400 MHz, DMSO-d₆) δ 9.31 (d, J = 2.4 Hz, 1H), |
| | fluorophenyl)(methyl)((2- | 9.21 (s, 1H), 8.64 (dd, J = 4.6, 1.2 Hz, 1H), 8.45 (dq, J = 8.4, |
| | (pyridin-3-yl)-2H- | 1.3 Hz, 1H), 8.14 (d, J = 8.9 Hz, 1H), 7.69 (dd, J = 7.9, 2.8 Hz, |
| | pyrazolo[4,3-b]pyridin- | 1H), 7.64 (dd, J = 7.9, 5.2 Hz, 3H), 7.49-7.47 (m, 1H), 6.91 (d, |
| | 5-yl)imino)-λ⁶- | J = 9.2 Hz, 1H), 3.38-3.39 (3H); ESI MS(m/z): 367.95[M + 1]. |
| | sulfanone | |
| 268 | ethyl((2-(5- | ¹H-NMR (400 MHz, DMSO-d₆) δ 9.21 (s, 1H), 9.08 (d, J = |
| | fluoropyridin-3-yl)-6- | 0.7 Hz, 1H), 8.70 (d, J = 2.4 Hz, 1H), 8.46 (dt, J = 10.0, 2.3 |
| | (trifluoromethyl)-2H- | Hz, 1H), 8.08 (s, 1H), 7.44 (s, 1H), 3.58 (t, J = 6.7 Hz, 1H), |
| | indazol-5- | 3.36 (t, J = 7.6 Hz, 2H), 1.36 (q, J = 6.7 Hz, 6H), 1.23 (t, J = |
| | yl)imino)(isopropyl)-λ⁶- | 7.5 Hz, 3H); ESI MS(m/z): 415[M + 1]. |
| | sulfanone | |
| 269 | diethyl((2-(5- | ¹H-NMR (400 MHz, DMSO-d₆) δ 9.21 (s, 1H), 9.09 (d, J = |
| | fluoropyridin-3-yl)-6- | 0.7 Hz, 1H), 8.70 (d, J = 2.2 Hz, 1H), 8.46 (dt, J = 9.9, 2.3 Hz, |
| | (trifluoromethyl)-2H- | 1H), 8.08 (s, 1H), 7.43 (s, 1H), 3.36 (q, J = 7.3 Hz, 4H), 1.26 |
| | indazol-5-yl)imino)-λ⁶- | (t, J = 7.3 Hz, 6H); ESI MS(m/z): 401.05[M + 1]. |
| | sulfanone | |
| 270 | ((2-(5-fluoropyridin-3- | ¹H-NMR (400 MHz, DMSO-d₆) δ 9.22 (s, 1H), 9.12 (d, J = |
| | yl)-6-(trifluoromethyl)- | 0.7 Hz, 1H), 8.71 (d, J = 2.2 Hz, 1H), 8.47 (dt, J = 9.9, 2.3 Hz, |
| | 2H-indazol-5- | 1H), 8.09 (s, 1H), 7.44 (s, 1H), 3.23 (s, 6H); ESI |
| | yl)imino)dimethyl-λ⁶- | MS(m/z): 373[M + 1]. |
| | sulfanone | |
| 271 | dipropyl((2-(pyridin-3- | ¹H-NMR (400 MHz, DMSO-d₆) δ 9.26 (d, J = 2.4 Hz, 1H), |
| | yl)-7-(trifluoromethyl)- | 9.13 (s, 1H), 8.66 (dd, J = 4.6, 1.5 Hz, 1H), 8.42 (dq, J = 8.3, |
| | 2H-indazol-5- | 1.4 Hz, 1H), 7.65 (dd, J = 8.3, 5.4 Hz, 1H), 7.46 (s, 1H), 7.33 |
| | yl)imino)-λ⁶-sulfanone | (d, J = 1.0 Hz, 1H), 3.33 (d, J = 6.1 Hz, 2H), 3.30 (s, 2H), |
| | | 1.80-1.72 (m, 4H), 0.97 (t, J = 7.5 Hz, 6H); ESI MS(m/z): 411 |
| | | [M + 1]. |
| 272 | dimethyl((2-(pyridin-3- | ¹H-NMR (400 MHz, DMSO-d₆) δ 9.27 (d, J = 2.2 Hz, 1H), |
| | yl)-7-(trifluoromethyl)- | 9.14 (s, 1H), 8.66 (dd, J = 4.8, 1.3 Hz, 1H), 8.43 (dq, J = 8.3, |
| | 2H-indazol-5- | 1.4 Hz, 1H), 7.65 (dd, J = 8.3, 4.6 Hz, 1H), 7.47 (d, J = 1.2 Hz, |
| | yl)imino)-λ⁶-sulfanone | 1H), 7.31 (d, J = 0.7 Hz, 1H), 3.27 (s, 6H); ESI MS(m/z): |
| | | 354.85 [M + 1]. |
| 273 | dimethyl((3-methyl-2- | ¹H-NMR (400 MHz, DMSO-d₆) δ 8.89 (s, 1H), 8.70 (d, J = |
| | (pyridin-3-yl)-2H- | 4.6 Hz, 1H), 8.12 (dt, J = 8.2, 1.2 Hz, 1H), 7.64 (dd, J = 7.5, |
| | indazol-5-yl)imino)-λ⁶- | 4.7 Hz, 1H), 7.47 (d, J = 9.2 Hz, 1H), 7.10 (s, 1H), 6.96 (d, J = |
| | sulfanone | 9.2 Hz, 1H), 3.21 (dd, J = 17.3, 16.0 Hz, 6H), 2.59 (s, 3H) |
| | | ESI MS(m/z): 301.05[M + 1]. |
| 274 | dipropyl((2-(pyridin-3- | ¹H-NMR (400 MHz, DMSO-d₆) δ 9.28 (d, J = 2.2 Hz, 1H), |
| | yl)-6-(trifluoromethyl)- | 9.05 (d, J = 0.7 Hz, 1H), 8.66 (dd, J = 4.6, 1.5 Hz, 1H), 8.44 |
| | 2H-indazol-5- | (dq, J = 8.3, 1.4 Hz, 1H), 8.08 (s, 1H), 7.66-7.63 (m, 1H), 7.45 |
| | yl)imino)-λ⁶-sulfanone | (s, 1H), 3.33 (d, J = 7.6 Hz, 2H), 3.28 (s, 2H), 1.81-1.71 (m, |
| | | 4H), 0.96 (t, J = 7.5 Hz, 6H); ESI MS(m/z): 410.95[M + 1] |
| 275 | ((2-(5-fluoropyridin-3- | ¹H-NMR (400 MHz, DMSO-d₆) δ 9.21 (s, 1H), 9.09 (d, J = |
| | yl)-6-(trifluoromethyl)- | 0.7 Hz, 1H), 8.70 (d, J = 2.2 Hz, 1H), 8.46 (dt, J = 10.0, 2.3 |
| | 2H-indazol-5- | Hz, 1H), 8.08 (s, 1H), 7.43 (s, 1H), 3.33 (s, 2H), 3.28 (d, J = |
| | yl)imino)dipropyl-λ⁶- | 7.1 Hz, 2H), 1.81-1.71 (m, 4H), 0.96 (t, J = 7.3 Hz, 6H) |
| | sulfanone | ESI MS(m/z): 428.95[M + 1]. |
| 276 | ethyl(isopropyl)((3- | ¹H-NMR (400 MHz, DMSO-d₆) δ 8.90 (s, 1H), 8.70 (d, J = |
| | methyl-2-(pyridin-3- | 4.6 Hz, 1H), 8.13 (d, J = 6.7 Hz, 1H), 7.64 (dd, J = 7.6, 4.3 Hz, |
| | yl)-2H-indazol-5- | 1H), 7.46 (d, J = 9.2 Hz, 1H), 7.11 (s, 1H), 7.03 (d, J = 9.2 Hz, |
| | yl)imino)-λ⁶-sulfanone | 1H), 3.56-3.49 (m, 1H), 3.26 (t, J = 7.3 Hz, 2H), 2.62 (d, J = |
| | | 30.3 Hz, 3H), 1.38-1.22 (m, 9H); ESI MS(m/z): 343.15[M + 1]. |
| 277 | isopropyl((3-methyl-2- | ¹H-NMR (400 MHz, DMSO-d₆) δ 8.89 (d, J = 2.4 Hz, 1H), |
| | (pyridin-3-yl)-2H- | 8.69 (dd, J = 4.7, 1.4 Hz, 1H), 8.14-8.11 (m, 1H), 7.63 (dd, J = |
| | indazol-5- | 8.1, 4.7 Hz, 1H), 7.45 (d, J = 9.2 Hz, 1H), 7.11 (d, J = 1.2 Hz, |
| | yl)imino)(propyl)-λ⁶- | 1H), 7.02 (dd, J = 9.2, 1.8 Hz, 1H), 3.54-3.47 (m, 1H), 3.26- |
| | sulfanone | 3.19 (m, 2H), 2.58 (s, 3H), 1.74 (dt, J = 23.1, 7.4 Hz, 2H), |
| | | 1.37 (d, J = 6.7 Hz, 3H), 1.31 (d, J = 7.0 Hz, 3H), 0.97 (t, J = |
| | | 7.2 Hz, 3H); ESI MS(m/z): 357.05[M + 1]. |
| 278 | dipropyl((2-(pyrimidin- | ¹H-NMR (400 MHz, DMSO-d₆) δ 9.53 (s, 2H), 9.29 (s, 1H), |
| | 5-yl)-2H-pyrazolo[3,4- | 9.08-9.07 (m, 2H), 6.83 (d, J = 1.2 Hz, 1H), 3.48-3.41 (m, |
| | c]pyridin-5-yl)imino)- | 4H), 1.84-1.68 (m, 4H), 0.98 (t, J = 7.5 Hz, 6H) |
| | λ⁶-sulfanone | ESI MS(m/z): 345.15[M + 1]. |
| 279 | ((2-(5-fluoropyridin-3- | ¹H-NMR (400 MHz, DMSO-d₆) δ 9.25 (s, 1H), 9.07-9.05 (m, |
| | yl)-2H-pyrazolo[3,4- | 2H), 8.72 (d, J = 2.4 Hz, 1H), 8.52 (dt, J = 9.9, 2.4 Hz, 1H), |
| | c]pyridin-5- | 6.81 (d, J = 1.2 Hz, 1H), 3.48-3.40 (m, 4H), 1.84-1.68 (m, |
| | yl)imino)dipropyl-λ⁶- | 4H), 0.98 (t, J = 7.3 Hz, 6H); ESI MS (m/z): 362.1[M + 1]. |
| | sulfanone | |
| 280 | diisopropyl((3-methyl- | ¹H-NMR (400 MHz, DMSO-d₆) δ 8.89 (d, J = 2.1 Hz, 1H), |
| | 2-(pyridin-3-yl)-2H- | 8.69 (dd, J = 4.7, 1.4 Hz, 1H), 8.13 (dq, J = 8.1, 1.4 Hz, 1H), |
| | indazol-5-yl)imino)-λ⁶- | 7.65-7.61 (m, 1H), 7.43 (dd, J = 9.0, 0.8 Hz, 1H), 7.12-7.08 |
| | sulfanone | (m, 2H), 3.62-3.56 (m, 2H), 2.57 (s, 3H), 1.34 (d, J = 7.0 Hz, |
| | | 6H), 1.27 (d, J = 6.7 Hz, 6H); ESI MS(m/z): 357.05[M + 1]. |
| 281 | ((3-methyl-2-(pyridin- | ¹H-NMR (400 MHz, DMSO-d₆) δ 8.89 (d, J = 2.4 Hz, 1H), |
| | 3-yl)-2H-indazol-5- | 8.70 (dd, J = 4.7, 1.4 Hz, 1H), 8.13 (dq, J = 8.3, 1.4 Hz, 1H), |
| | yl)imino)dipropyl-λ⁶- | 7.65-7.62 (m, 1H), 7.46 (dd, J = 9.2, 0.6 Hz, 1H), 7.09 (t, J = |
| | sulfanone | 0.9 Hz, 1H), 6.99 (dd, J = 9.2, 1.8 Hz, 1H), 3.28-3.24 (m, 4H), |
| | | 2.58 (s, 3H), 1.79-1.71 (m, 4H), 0.96 (t, J = 7.3 Hz, 6H) |
| | | ESI MS(m/z): 357.10[M + 1]. |
| 282 | diethyl((3-methyl-2- | ¹H-NMR (400 MHz, DMSO-d₆) δ 8.89 (d, J = 2.1 Hz, 1H), |
| | (pyridin-3-yl)-2H- | 8.70 (dd, J = 4.7, 1.4 Hz, 1H), 8.14-8.11 (m, 1H), 7.64 (dd, J = |
| | indazol-5-yl)imino)-λ⁶- | 8.1, 4.7 Hz, 1H), 7.46 (d, J = 9.2 Hz, 1H), 7.11 (d, J = 1.2 Hz, |
| | sulfanone | 1H), 7.00 (dd, J = 9.0, 2.0 Hz, 1H), 3.34-3.25 (m, 4H), 2.58 (s, |
| | | 3H), 1.26 (t, J = 7.5 Hz, 6H); ESI MS(m/z): 328.9[M + 1]. |
| 283 | isopropyl(methyl)((3- | ¹H-NMR (400 MHz, DMSO-d₆) δ 8.89 (d, J = 2.1 Hz, 1H), |
| | methyl-2-(pyridin-3- | 8.70 (dd, J = 4.6, 1.5 Hz, 1H), 8.13 (dq, J = 8.1, 1.4 Hz, 1H), |
| | yl)-2H-indazol-5- | 7.64 (dd, J = 8.1, 4.7 Hz, 1H), 7.47 (d, J = 9.2 Hz, 1H), 7.11 |
| | yl)imino)-λ⁶-sulfanone | (d, J = 1.2 Hz, 1H), 7.00 (dd, J = 9.0, 2.0 Hz, 1H), 3.50 (t, J = |
| | | 6.9 Hz, 1H), 3.02 (s, 3H), 2.59 (s, 3H), 1.37 (dd, J = 19.7, 6.9 |
| | | Hz, 6H); ESI MS(m/z): 328.9[M + 1]. |
| 284 | ethyl(methyl)((3- | ¹H-NMR (400 MHz, DMSO-d₆) δ 8.89 (d, J = 2.4 Hz, 1H), |
| | methyl-2-(pyridin-3- | 8.70 (dd, J = 4.7, 1.4 Hz, 1H), 8.13 (dq, J = 8.1, 1.4 Hz, 1H), |
| | yl)-2H-indazol-5- | 7.64 (dd, J = 8.3, 4.9 Hz, 1H), 7.48-7.46 (m, 1H), 7.10 (d, J = |
| | yl)imino)-λ⁶-sulfanone | 1.2 Hz, 1H), 6.98 (dd, J = 9.2, 1.8 Hz, 1H), 3.35 (q, J = 7.2 Hz, |
| | | 2H), 3.10 (s, 3H), 2.59 (s, 3H), 1.29 (t, J = 7.3 Hz, 3H) |
| | | ESI MS(m/z): 315.0[M + 1]. |
| 285 | dipropyl((2-(pyridin-3- | ¹H-NMR (400 MHz, DMSO-d₆) δ 9.32 (d, J = 2.4 Hz, 1H), |
| | yl)-2H-pyrazolo[3,4- | 9.05 (t, J = 1.2 Hz, 1H), 9.01 (d, J = 0.9 Hz, 1H), 8.68 (dd, J = |
| | c]pyridin-5-yl)imino)- | 4.7, 1.4 Hz, 1H), 8.48 (dq, J = 8.4, 1.3 Hz, 1H), 7.65 (q, J = |
| | λ⁶-sulfanone | 4.3 Hz, 1H), 6.82 (d, J = 1.5 Hz, 1H), 3.44 (t, J = 7.9 Hz, 4H), |
| | | 1.82-1.70 (m, 4H), 0.98 (t, J = 7.5 Hz, 6H); ESI |
| | | MS(m/z): 344.05 [M + 1]. |
| 286 | methyl(propyl)((2- | ¹H-NMR (400 MHz, DMSO-d₆) δ 9.32 (d, J = 2.4 Hz, 1H), |
| | (pyridin-3-yl)-2H- | 9.06 (t, J = 1.1 Hz, 1H), 9.01 (d, J = 0.9 Hz, 1H), 8.68 (dd, J = |
| | pyrazolo[3,4-c]pyridin- | 4.6, 1.5 Hz, 1H), 8.49 (dq, J = 8.4, 1.3 Hz, 1H), 7.67-7.63 (m, |
| | 5-yl)imino)-λ⁶- | 1H), 6.81 (d, J = 1.5 Hz, 1H), 3.51-3.45 (m, 2H), 3.28 (s, 3H), |
| | sulfanone | 1.84-1.73 (m, 2H), 0.99 (t, J = 7.5 Hz, 3H); ESI MS(m/z): |
| | | 316[M + 1]. |
| 287 | sec-butyl(isopropyl)((2- | ¹H-NMR (400 MHz, DMSO-d₆) δ 9.27 (d, J = 2.4 Hz, 1H), |
| | (pyridin-3-yl)-2H- | 9.00 (s, 1H), 8.59 (dd, J = 4.7, 1.4 Hz, 1H), 8.42 (dq, J = 8.4, |
| | pyrazolo[4,3-b]pyridin- | 1.3 Hz, 1H), 7.92 (dd, J = 9.2, 0.6 Hz, 1H), 7.60 (dd, J = 8.3, |
| | 5-yl)imino)-λ⁶- | 4.9 Hz, 1H), 6.84 (dd, J = 9.2, 0.6 Hz, 1H), 3.87 (td, J = 13.0, |
| | sulfanone | 6.4 Hz, 1H), 3.78-3.71 (m, 1H), 2.15-2.05 (m, 1H), 1.68-1.53 |
| | | (m, 1H), 1.43-1.37 (m, 9H), 0.98 (q, J = 7.1 Hz, 3H) |
| | | ESI MS(m/z): 357.95[M + 1]. |
| 288 | (cyclopropylmethyl)(iso- | ¹H-NMR (400 MHz, DMSO-d₆) δ 9.27 (d, J = 2.1 Hz, 1H), |
| | propyl)((2-(pyridin-3- | 9.02 (d, J = 0.6 Hz, 1H), 8.60 (dd, J = 4.7, 1.4 Hz, 1H), 8.41 |
| | yl)-2H-pyrazolo[4,3- | (dq, J = 8.3, 1.4 Hz, 1H), 7.94 (dd, J = 9.2, 0.6 Hz, 1H), 7.62- |
| | b]pyridin-5-yl)imino)- | 7.58 (m, 1H), 6.81 (d, J = 9.2 Hz, 1H), 3.82 (t, J = 6.9 Hz, |
| | λ⁶-sulfanone | 1H), 3.70 (dq, J = 25.9, 7.2 Hz, 2H), 1.39 (dd, J = 8.4, 6.9 Hz, |
| | | 6H), 1.07-1.05 (m, 1H), 0.62-0.54 (m, 2H), 0.40-0.31 (m, 2H) |
| | | ESI MS(m/z): 355.95[M + 1]. |
| 289 | (cyclobutylmethyl)(iso- | ¹H-NMR (400 MHz, DMSO-d₆) δ 9.27 (d, J = 2.4 Hz, 1H), |
| | propyl)((2-(pyridin-3- | 9.02 (d, J = 0.9 Hz, 1H), 8.59 (dd, J = 4.7, 1.4 Hz, 1H), 8.41 |
| | yl)-2H-pyrazolo[4,3- | (dq, J = 8.4, 1.3 Hz, 1H), 7.93 (dd, J = 9.2, 0.9 Hz, 1H), 7.60 |
| | b]pyridin-5-yl)imino)- | (ddd, J = 8.4, 4.7, 0.6 Hz, 1H), 6.78 (d, J = 9.2 Hz, 1H), 3.85- |
| | λ⁶-sulfanone | 3.67 (m, 3H), 2.12-2.02 (m, 2H), 1.90-1.73 (m, 5H), 1.35 (dd, |
| | | J = 6.7, 4.9 Hz, 6H); ESI MS(m/z): 370 [M + 1]. |
| 290 | 1-((2-(pyridin-3-yl)-2H- | ¹H-NMR (400 MHz, DMSO-d₆) δ 9.26 (d, J = 2.4 Hz, 1H), |
| | pyrazolo[4,3-b]pyridin- | 9.01 (d, J = 0.6 Hz, 1H), 8.60 (dd, J = 4.7, 1.4 Hz, 1H), 8.40 |
| | 5-yl)imino)hexahydro- | (dq, J = 8.5, 1.4 Hz, 1H), 7.96 (dd, J = 9.2, 0.9 Hz, 1H), 7.61 |
| | λ⁶-thiopyran 1-oxide | (dd, J = 8.4, 4.7 Hz, 1H), 6.81 (d, J = 9.2 Hz, 1H), 3.75-3.71 |
| | | (m, 2H), 3.49-3.43 (m, 2H), 1.99-1.81 (m, 2H), 1.63-1.58 (m, |
| | | 2H), 1.25-1.22 (m, 2H); ESI MS(m/z): 327.9[M + 1]. |
| 291 | sec-butyl(isopropyl)((2- | ¹H-NMR (400 MHz, DMSO-d₆) δ 9.25 (d, J = 2.4 Hz, 1H), |
| | (pyridin-3-yl)-2H- | 8.89 (d, J = 0.6 Hz, 1H), 8.59 (dd, J = 4.6, 1.5 Hz, 1H), 8.40 |
| | indazol-5-yl)imino)-λ⁶- | (dq, J = 8.3, 1.4 Hz, 1H), 7.60 (dd, J = 8.1, 5.0 Hz, 1H), 7.53 |
| | sulfanone | (d, J = 9.2 Hz, 1H), 7.18 (t, J = 2.0 Hz, 1H), 7.07 (dt, J = 9.2, |
| | | 2.4 Hz, 1H), 3.37 (qd, J = 6.8, 3.2 Hz, 1H), 3.27-3.17 (m, 1H), |
| | | 1.35-1.32 (m, 5H), 1.27 (t, J = 7.0 Hz,3H), 1.20-1.17 (m, 3H), |
| | | 0.98-0.90 (m, 3H); ESI MS(m/z): 356.95[M + 1]. |
| 292 | 1-((2-(pyridin-3-yl)-2H- | ¹H-NMR (400 MHz, DMSO-d₆) δ 9.26 (d, J = 2.4 Hz, 1H), |
| | indazol-5- | 8.91 (d, J = 0.6 Hz, 1H), 8.60 (dd, J = 4.7, 1.4 Hz, 1H), 8.41 |
| | yl)imino)hexahydro-λ⁶- | (dq, J = 8.4, 1.3 Hz, 1H), 7.62-7.60 (m, 1H), 7.58 (d, J = 9.2 |
| | thiopyran 1-oxide | Hz, 1H), 7.17 (d, J = 1.2 Hz, 1H), 7.03 (dd, J = 9.2, 1.8 Hz, |
| | | 1H), 3.38 (t, J = 6.7 Hz, 2H), 3.27-3.19 (m, 2H), 1.90 (q, J = |
| | | 5.5 Hz, 4H), 1.64-1.55 (m, 2H); ESI MS(m/z): 326.9 [M + 1]. |
| 293 | tert-butyl((2-(5- | ¹H-NMR (400 MHz, DMSO-d₆) δ 9.20 (d, J = 1.2 Hz, 1H), |
| | fluoropyridin-3-yl)-2H- | 8.97 (d, J = 1.0 Hz, 1H), 8.63 (d, J = 2.7 Hz, 1H), 8.42 (dt, J = |
| | indazol-5- | 10.2, 2.3 Hz, 1H), 7.56 (d, J = 9.0 Hz, 1H), 7.16 (q, J = 0.9 Hz, |
| | yl)imino)(methyl)-λ⁶- | 1H), 7.03 (dd, J = 9.2, 2.1 Hz, 1H), 3.01 (s, 3H), 1.43 (s, 9H) |
| | sulfanone | ESI MS(m/z): 346.9[M + 1]. |
| 294 | isopentyl(isopropyl)((2- | ¹H-NMR (400 MHz, DMSO-d₆) δ 9.47 (s, 2H), 9.21 (s, 1H), |
| | (pyrimidin-5-yl)-2H- | 8.97 (d, J = 0.9 Hz, 1H), 7.57 (d, J = 9.2 Hz, 1H), 7.18 (t, J = |
| | indazol-5-yl)imino)-λ⁶- | 1.1 Hz, 1H), 7.05 (dd, J = 9.2, 1.8 Hz, 1H), 3.54 (t, J = 6.7 Hz, |
| | sulfanone | 1H), 3.26-3.23 (m, 2H), 1.65-1.52 (m,3H), 1.37 (d, J = 6.7 Hz, |
| | | 3H), 1.21 (q, J = 3.3 Hz, 3H), 0.84 (dd, J = 7.8, 6.3 Hz, 6H) |
| | | ESI MS(m/z): 371.95[M + 1]. |
| 295 | ethyl(methyl)((2- | ¹H-NMR (400 MHz, DMSO-d₆) δ 9.27 (d, J = 2.7 Hz, 1H), |
| | (pyridin-3-yl)-7- | 9.14 (s, 1H), 8.66 (dd, J = 4.6, 1.5 Hz, 1H), 8.42 (dq, J = 8.3, |
| | (trifluoromethyl)-2H- | 1.4 Hz, 1H), 7.65 (dd, J = 8.3, 5.4 Hz, 1H), 7.47 (s, 1H), 7.33 |
| | indazol-5-yl)imino)-λ⁶- | (s, 1H), 3.41 (q, J = 7.3 Hz, 2H), 3.17 (s, 3H), 1.31 (t, J = 7.3 |
| | sulfanone | Hz, 3H); ESI MS(m/z): 369[M + 1]. |
| 296 | diethyl((2-(pyridin-3- | ¹H-NMR (400 MHz, DMSO-d₆) δ 9.26 (d, J = 2.4 Hz, 1H), |
| | yl)-7-(trifluoromethyl)- | 9.13 (s, 1H), 8.66 (dd, J = 4.7, 1.4 Hz, 1H), 8.42 (dq, J = 8.3, |
| | 2H-indazol-5- | 1.4 Hz, 1H), 7.66-7.63 (m, 1H), 7.47 (d, J = 0.9 Hz, 1H), 7.35 |
| | yl)imino)-λ⁶-sulfanone | (t, J = 0.9 Hz, 1H), 3.36 (q, J = 7.4 Hz, 4H), 1.27 (t, J = 7.5 |
| | | Hz, 6H); ESI MS(m/z): 383[M + 1]. |
| 297 | ethyl(methyl)((2- | ¹H-NMR (400 MHz, DMSO-d₆) δ 9.50 (s, 2H), 9.28 (s, 1H), |
| | (pyrimidin-5-yl)-6- | 9.13 (s, 1H), 8.11 (s, 1H), 7.46 (s, 1H), 3.40 (q, J = 7.4 Hz, |
| | (trifluoromethyl)-2H- | 2H), 3.14 (s, 3H), 1.30 (t, J = 7.3 Hz, 3H); ESI |
| | indazol-5-yl)imino)-λ⁶- | MS(m/z): 369.9[M + 1]. |
| | sulfanone | |
| 298 | ethyl(methyl)((2- | ¹H-NMR (400 MHz, DMSO-d₆) δ 9.29 (d, J = 2.2 Hz, 1H), |
| | (pyridin-3-yl)-6- | 9.07 (d, J = 0.7 Hz, 1H), 8.66 (dd, J = 4.6, 1.5 Hz, 1H), 8.44 |
| | (trifluoromethyl)-2H- | (dq, J = 8.5, 1.3 Hz, 1H), 8.09 (s, 1H), 7.67-7.63 (m, 1H), 7.46 |
| | indazol-5-yl)imino)-λ⁶- | (s, 1H), 3.39 (q, J = 7.3 Hz, 2H), 3.13 (s, 3H), 1.31 (t, J = 7.3 |
| | sulfanone | Hz, 3H); ESI MS(m/z): 368.85[M + 1]. |
| 299 | 4-((2-(pyridin-3-yl)-6- | ¹H-NMR (400 MHz, DMSO-d₆) δ 9.29 (d, J = 2.4 Hz, 1H), |
| | (trifluoromethyl)-2H- | 9.08 (d, J = 0.6 Hz, 1H), 8.67 (dd, J = 4.6, 1.5 Hz, 1H), 8.44 |
| | indazol-5-yl)imino)- | (dq, J = 8.3, 1.4 Hz, 1H), 8.13 (s, 1H), 7.65 (dd, J = 8.4, 4.7 |
| | 1,4λ⁶-oxathiane 4-oxide | Hz, 1H), 7.47 (s, 1H), 4.11-4.07 (m, 2H), 3.84 (t, J = 10.4 Hz, |
| | | 2H), 3.63 (d, J = 13.1 Hz, 2H), 3.49-3.42 (m, 2H); ESI |
| | | MS(m/z): 396.9[M + 1]. |
| 300 | ethyl((2-(5- | ¹H-NMR (400 MHz, DMSO-d₆) δ 9.22 (s, 1H), 9.11 (s, 1H), |
| | fluoropyridin-3-yl)-6- | 8.70 (d, J = 2.4 Hz, 1H), 8.47 (dt, J = 10.0, 2.3 Hz, 1H), 8.09 |
| | (trifluoromethyl)-2H- | (s, 1H), 7.44 (s, 1H), 3.40 (q, J = 7.3 Hz, 2H), 3.14 (s, 3H), |
| | indazol-5- | 1.30 (t, J = 7.3 Hz, 3H); ESI MS(m/z): 386.85[M + 1]. |
| | yl)imino)(methyl)-λ⁶- | |
| | sulfanone | |
| 301 | ((2-(5-fluoropyridin-3- | ¹H-NMR (400 MHz, DMSO-d₆) δ 9.22 (s, 1H), 9.12 (d, J = |
| | yl)-6-(trifluoromethyl)- | 0.7 Hz, 1H), 8.71 (d, J = 2.4 Hz, 1H), 8.47 (dt, J = 9.9, 2.4 Hz, |
| | 2H-indazol-5- | 1H), 8.10 (s, 1H), 7.45 (s, 1H), 3.80-3.62 (m, 4H), 3.27 (s, |
| | yl)imino)(2- | 3H), 3.18 (s, 3H); ESI MS(m/z): 416.9[M + 1]. |
| | methoxyethyl)(methyl)- | |
| | λ⁶-sulfanone | |
| 302 | 4-((2-(5-fluoropyridin- | ¹H-NMR (400 MHz, DMSO-d₆) δ 9.22 (s, 1H), 9.12 (s, 1H), |
| | 3-yl)-6- | 8.71 (d, J = 2.4 Hz, 1H), 8.49-8.45 (m, 1H), 8.13 (s, 1H), 7.45 |
| | (trifluoromethyl)-2H- | (s, 1H), 4.11-4.08 (m, 2H), 3.84 (t, J = 10.4 Hz, 2H), 3.64 (d, |
| | indazol-5-yl)imino)- | J = 13.7 Hz, 2H), 3.50-3.43 (m, 2H); ESI MS(m/z): 414.9[M + 1]. |
| | 1,4λ⁶-oxathiane 4-oxide | |
| 303 | 1-((2-(5-fluoropyridin- | ¹H-NMR (400 MHz, DMSO-d₆) δ 9.22 (s, 1H), 9.13 (d, J = |
| | 3-yl)-6- | 1.0 Hz, 1H), 8.71 (d, J = 2.4 Hz, 1H), 8.47 (dt, J = 10.0, 2.3 |
| | (trifluoromethyl)-2H- | Hz, 1H), 8.10 (s, 1H), 7.36 (s, 1H), 3.37 (q, J = 6.6 Hz, 2H), |
| | indazol-5- | 3.29-3.24 (m, 2H), 2.26-2.19 (m, 2H), 2.11-2.05 (m, 2H) |
| | yl)imino)tetrahydro- | ESI MS(m/z): 398.9[M + 1]. |
| | 1H-λ⁶-thiophene 1- | |
| | oxide | |
| 304 | 1-((3-methyl-2- | ¹H-NMR (400 MHz, DMSO-d₆) δ 8.90 (d, J = 2.1 Hz, 1H), |
| | (pyridin-3-yl)-2H- | 8.70 (d, J = 3.7 Hz, 1H), 8.13 (dq, J = 8.3, 1.3 Hz, 1H), 7.64 |
| | indazol-5- | (dd, J = 8.3, 4.9 Hz, 1H), 7.49 (d, J = 9.2 Hz, 1H), 7.06 (d, J = |
| | yl)imino)tetrahydro- | 1.2 Hz, 1H), 6.97 (dd, J = 9.0, 2.0 Hz, 1H), 3.36 (q, J = 6.5 Hz, |
| | 1H-λ⁶-thiophene 1- | 2H), 3.26-3.20 (m, 2H), 2.60 (s, 3H), 2.24-2.10 (m, 4H) |
| | oxide | ESI MS(m/z): 327.00[M + 1]. |
| 305 | dipropyl((2-(pyrimidin- | ¹H-NMR (400 MHz, DMSO-d₆) δ 9.50 (s, 2H), 9.27 (s, 1H), |
| | 5-yl)-6- | 9.11 (d, J = 0.9 Hz, 1H), 8.10 (s, 1H), 7.45 (s, 1H), 3.34-3.32 |
| | (trifluoromethyl)-2H- | (m, 4H), 1.79-1.71 (m, 4H), 0.96 (t, J = 7.3 Hz, 6H) |
| | indazol-5-yl)imino)-λ⁶- | ESI MS(m/z): 411.9[M + 1]. |
| | sulfanone | |
| 306 | ((2-(5-fluoropyridin-3- | ¹H-NMR (400 MHz, DMSO-d₆) δ 9.20 (s, 1H), 9.07 (d, J = |
| | yl)-2H-pyrazolo[4,3- | 1.0 Hz, 1H), 8.63 (d, J = 2.2 Hz, 1H), 8.41 (dt, J = 10.2, 2.3 |
| | b]pyridin-5- | Hz, 1H), 7.96 (dd, J = 9.2, 0.9 Hz, 1H), 6.78 (d, J = 9.0 Hz, |
| | yl)imino)dimethyl-λ⁶- | 1H), 3.44 (s, 6H); ESI MS (m/z): 305.75[M + 1]. |
| | sulfanone | |
| 307 | ethyl((2-(5- | ¹H-NMR (400 MHz, DMSO-d₆) δ 9.20 (s, 1H), 9.06 (d, J = |
| | fluoropyridin-3-yl)-2H- | 0.7 Hz, 1H), 8.63 (d, J = 2.4 Hz, 1H), 8.41 (dt, J = 10.0, 2.3 |
| | pyrazolo[4,3-b]pyridin- | Hz, 1H), 7.95 (dd, J = 9.2, 0.9 Hz, 1H), 6.79 (d, J = 9.3 Hz, |
| | 5-yl)imino)(methyl)-λ⁶- | 1H), 3.60 (q, J = 7.4 Hz, 2H), 3.36 (s, 3H), 1.31 (t, J = 7.5 Hz, |
| | sulfanone | 3H); p ESI MS (m/z): 319.80[M + 1]. |
| 308 | diethyl((2-(5- | ¹H-NMR (400 MHz, DMSO-d₆) δ 9.19 (s, 1H), 9.05 (d, J = |
| | fluoropyridin-3-yl)-2H- | 0.7 Hz, 1H), 8.63 (d, J = 2.4 Hz, 1H), 8.41 (dt, J = 10.0, 2.3 |
| | pyrazolo[4,3-b]pyridin- | Hz, 1H), 7.95 (dd, J = 9.2, 0.9 Hz, 1H), 6.81 (d, J = 9.3 Hz, |
| | 5-yl)imino)-λ⁶- | 1H), 3.58 (q, J = 7.3 Hz, 4H), 1.28 (t, J = 7.3 Hz, 6H) |
| | sulfanone | ESI MS (m/z): 333.85[M + 1]. |
| 309 | ((2-(5-fluoropyridin-3- | ¹H-NMR (400 MHz, DMSO-d₆) δ 9.20 (s, 1H), 9.07 (d, J = |
| | yl)-2H-pyrazolo[4,3- | 0.7 Hz, 1H), 8.63 (d, J = 2.4 Hz, 1H), 8.41 (dt, J = 10.2, 2.3 |
| | b]pyridin-5- | Hz, 1H), 7.95 (dd, J = 9.2, 0.9 Hz, 1H), 6.78 (d, J = 9.0 Hz, |
| | yl)imino)(methyl)(propyl)- | 1H), 3.61-3.53 (m, 2H), 3.38 (s, 3H), 1.85-1.73 (m, 2H), 1.00 |
| | λ⁶-sulfanone | (t, J = 7.5 Hz, 3H); ESI MS (m/z): 333.80[M + 1]. |
| 310 | ((2-(5-fluoropyridin-3- | ¹H-NMR (400 MHz, DMSO-d₆) δ 9.20 (s, 1H), 9.07 (d, J = |
| | yl)-2H-pyrazolo[4,3- | 1.0 Hz, 1H), 8.63 (d, J = 2.7 Hz, 1H), 8.42 (dt, J = 10.3, 2.3 |
| | b]pyridin-5- | Hz, 1H), 7.94 (dd, J = 9.2, 0.9 Hz, 1H), 6.79 (d, J = 9.3 Hz, |
| | yl)imino)dipropyl-λ6- | 1H), 3.59-3.50 (m, 4H), 1.85-1.69 (m, 4H), 0.99 (t, J = 7.5 Hz, |
| | sulfanone | 6H); ESI MS (m/z): 362.20[M + 1]. |
| 311 | ((2-(5-fluoropyridin-3- | ¹H-NMR (400 MHz, DMSO-d₆) δ 9.19 (s, 1H), 9.07 (d, J = |
| | yl)-2H-pyrazolo[4,3- | 1.0 Hz, 1H), 8.63 (d, J = 2.4 Hz, 1H), 8.41 (dt, J = 10.2, 2.3 |
| | b]pyridin-5- | Hz, 1H), 7.94 (dd, J = 9.2, 0.9 Hz, 1H), 6.80 (d, J = 9.0 Hz, |
| | yl)imino)(isopropyl)(methyl)- | 1H), 3.37 (d, J = 8.8 Hz, 3H), 1.45-1.37 (m, 6H); ESI MS |
| | λ⁶-sulfanone | (m/z): 333.80[M + 1]. |
| 312 | ethyl((2-(5- | ¹H-NMR (400 MHz, DMSO-d₆) δ 9.19 (s, 1H), 9.06 (d, J = |
| | fluoropyridin-3-yl)-2H- | 0.7 Hz, 1H), 8.63 (d, J = 2.4 Hz, 1H), 8.41 (dt, J = 10.3, 2.3 |
| | pyrazolo[4,3-b]pyridin- | Hz, 1H), 7.94 (d, J = 9.0 Hz, 1H), 6.82 (d, J = 9.3 Hz, 1H), |
| | 5-yl)imino)(isopropyl)- | 3.93-3.87 (m, 1H), 3.69-3.59 (m, 2H), 1.37 (dd, J = 9.3, 6.8 |
| | λ⁶-sulfanone | Hz, 6H), 1.28 (t, J = 7.3 Hz, 3H); ESI MS (m/z): 348.00[M + 1]. |
| 313 | tert-butyl((2-(5- | ¹H-NMR (400 MHz, DMSO-d₆) δ 9.20 (s, 1H), 9.08 (d, J = |
| | fluoropyridin-3-yl)-2H- | 1.0 Hz, 1H), 8.63 (d, J = 2.4 Hz, 1H), 8.42 (dt, J = 10.2, 2.4 |
| | pyrazolo[4,3-b]pyridin- | Hz, 1H), 7.95-7.92 (m, 1H), 6.80 (d, J = 9.3 Hz, 1H), 3.46 (s, |
| | 5-yl)imino)(methyl)-λ⁶- | 3H), 1.46 (s, 9H); ESI MS (m/z): 347.85[M + 1]. |
| | sulfanone | |
| 314 | ethyl(methyl)((2- | ¹H-NMR (400 MHz, DMSO-d₆) δ 9.47 (s, 2H), 9.21 (s, 1H), |
| | (pyrimidin-5-yl)-2H- | 9.08 (d, J = 1.0 Hz, 1H), 7.97 (dd, J = 9.2, 0.9 Hz, 1H), 6.80 |
| | pyrazolo[4,3-b]pyridin- | (d, J = 9.3 Hz, 1H), 3.61 (q, J = 7.3 Hz, 2H), 3.37 (s, 3H), 1.31 |
| | 5-yl)imino)-λ⁶- | (t, J = 7.3 Hz, 3H); ESI MS (m/z): 302.80[M + 1]. |
| | sulfanone | |
| 315 | isopropyl(propyl)((2- | ¹H-NMR (400 MHz, DMSO-d₆) δ 9.47 (s, 2H), 9.20 (d, J = |
| | (pyrimidin-5-yl)-2H- | 4.9 Hz, 1H), 9.08 (d, J = 1.0 Hz, 1H), 7.96 (d, J = 9.3 Hz, 1H), |
| | pyrazolo[4,3-b]pyridin- | 6.83 (d, J = 9.3 Hz, 1H), 3.97-3.90 (m, 1H), 3.63-3.50 (m, |
| | 5-yl)imino)-λ⁶- | 2H), 1.87-1.68 (m, 2H), 1.37 (dd, J = 8.6, 6.8 Hz, 6H), 0.99 (t, |
| | sulfanone | J = 7.5 Hz, 3H); ESI MS (m/z): 344.85[M + 1]. |
| 316 | diisopropyl((2- | ¹H-NMR (400 MHz, DMSO-d₆) δ 9.47 (s, 2H), 9.20 (s, 1H), |
| | (pyrimidin-5-yl)-2H- | 9.05 (d, J = 0.7 Hz, 1H), 7.94 (dd, J = 9.2, 0.9 Hz, 1H), 6.87 |
| | pyrazolo[4,3-b]pyridin- | (d, J = 9.3 Hz, 1H), 3.97-3.91 (m, 2H), 1.40 (dd, J = 16.4, 6.8 |
| | 5-yl)imino)-λ⁶- | Hz, 12H); ESI MS (m/z): 344.85[M + 1]. |
| | sulfanone | |
| 317 | tert-butyl(methyl)((2- | ¹H-NMR (400 MHz, DMSO-d₆) δ 9.48 (s, 2H), 9.21 (s, 1H), |
| | (pyrimidin-5-yl)-2H- | 9.10 (d, J = 0.7 Hz, 1H), 7.96 (dd, J = 9.2, 0.9 Hz, 1H), 6.81 |
| | pyrazolo[4,3-b]pyridin- | (d, J = 9.3 Hz, 1H), 3.46 (s, 3H), 1.46 (s, 9H) |
| | 5-yl)imino)-λ⁶- | ESI MS (m/z): 330.85[M + 1]. |
| | sulfanone | |
| 318 | ((2-(5-fluoropyridin-3- | ¹H-NMR (400 MHz, DMSO-d₆) δ 9.20 (s, 1H), 9.08 (d, J = |
| | yl)-2H-pyrazolo[4,3- | 1.0 Hz, 1H), 8.64 (d, J = 2.4 Hz, 1H), 8.42 (dt, J = 10.2, 2.3 |
| | b]pyridin-5- | Hz, 1H), 7.97 (dd, J = 9.2, 0.9 Hz, 1H), 6.81 (d, J = 9.0 Hz, |
| | yl)imino)(2- | 1H), 3.99-3.83 (m, 2H), 3.82-3.75 (m, 2H), 3.37 (s, 3H), 3.33 |
| | methoxyethyl)(methyl)- | (s, 3H); ESI MS (m/z): 349.80[M + 1]. |
| | λ⁶-sulfanone | |
| 493 | (cyclobutylmethyl)(isopropyl)((2- | ¹H-NMR (400 MHz, DMSO-d₆) δ 9.26 (d, J = 2.4 Hz, 1H), 8.91 |
| | (pyridin-3- | (d, J = 0.9 Hz, 1H), 8.60 (dd, J = 4.7, 1.4 Hz, 1H), 8.41 (dq, J = |
| | yl)-2H-indazol-5- | 8.3, 1.4 Hz, 1H), 7.60 (dd, J = 8.1, 5.0 Hz, 1H), 7.55 (d, J = 9.2 |
| | yl)imino)-λ⁶-sulfanone | Hz, 1H), 7.16 (d, J = 1.2 Hz, 1H), 7.00 (dd, J = 9.2, 1.8 Hz, 1H), |
| | | 3.45-3.35 (m, 3H), 2.81-2.77 (m, 1H), 2.04 (t, J = 7.8 Hz, 2H), |
| | | 1.84-1.72 (m, 5H), 1.33 (dd, J = 26.6, 6.7 Hz, 7H) |
| | | ESI MS (m/z): 368.95 [M + 1] |
| 494 | sec-butyl(isopropyl)((2- | ¹H-NMR (400 MHz, DMSO-d₆) δ 9.46 (d, J = 2.1 Hz, 2H), 9.20 |
| | (pyrimidin-5-yl)-2H- | (s, 1H), 8.95 (d, J = 0.6 Hz, 1H), 7.55 (d, J = 9.2 Hz, 1H), 7.19 |
| | indazol-5-yl)imino)-λ⁶- | (t, J = 2.0 Hz, 1H), 7.10 (dt, J = 9.3, 2.4 Hz, 1H), 3.65-3.59 (m, |
| | sulfanone | 1H), 3.38 (tt, J = 10.2, 3.5 Hz, 1H), 2.07-2.00 (m, 1H), 1.35- |
| | | 1.32 (m, 5H), 1.29-1.22 (m, 5H), 0.94 (dt, J = 16.5, 7.5 Hz, 3H) |
| | | ESI MS (m/z): 357.9 [M + 1] |
| 495 | (cyclobutylmethyl)(isopropyl)((2- | ¹H-NMR (400 MHz, DMSO-d₆) δ 9.47 (s, 2H), 9.21 (s, 1H), |
| | (pyrimidin-5- | 8.97 (s, 1H), 7.57 (d, J = 9.2 Hz, 1H), 7.16 (d, J = 1.2 Hz, 1H), |
| | yl)-2H-indazol-5- | 7.02 (dd, J = 9.2, 1.8 Hz, 1H), 3.42 (dt, J = 18.8, 6.7 Hz, 3H), |
| | yl)imino)-λ⁶-sulfanone | 2.79 (s, 1H), 2.05 (d, J = 7.6 Hz, 2H), 1.84-1.73 (m, 4H), 1.33 |
| | | (dd, J = 26.6, 6.7 Hz, 6H); ESI MS (m/z): 369.9 [M + 1]. |
| 496 | sec-butyl((2-(5- | ¹H-NMR (400 MHz, DMSO-d₆) δ 9.18 (s, 1H), 8.93 (s, 1H), |
| | fluoropyridin-3-yl)-2H- | 8.62 (d, J = 2.4 Hz, 1H), 8.40 (dt, J = 10.2, 2.4 Hz, 1H), 7.53 |
| | indazol-5- | (d, J = 9.2 Hz, 1H), 7.17 (t, J = 2.0 Hz, 1H), 7.09 (dt, J = 9.2, |
| | yl)imino)(isopropyl)-λ⁶- | 2.3 Hz, 1H), 3.65-3.58 (m, 1H), 3.38 (qd, J = 6.8, 3.4 Hz, 1H), |
| | sulfanone | 2.06-2.00 (m, 1H), 1.56-1.37 (m, 1H), 1.35-1.32 (m, 4H), 1.27 |
| | | (t, J = 7.0 Hz, 5H), 0.94 (dt, J = 16.2, 7.5 Hz, 3H); ESI MS |
| | | (m/z): 375.1 [M + 1] |
| 497 | ((2-(5-fluoropyridin-3- | ¹H-NMR (400 MHz, DMSO-d₆) δ 9.19 (s, 1H), 8.95 (d, J = 0.6 |
| | yl)-2H-indazol-5- | Hz, 1H), 8.62 (d, J = 2.4 Hz, 1H), 8.41 (dt, J = 10.1, 2.3 Hz, |
| | yl)imino)(isopentyl)(isopropyl)- | 1H), 7.55 (d, J = 9.2 Hz, 1H), 7.16 (d, J = 1.2 Hz, 1H), 7.04 (dd, |
| | λ⁶-sulfanone | J = 9.2, 2.1 Hz, 1H), 3.58-3.51 (m, 1H), 3.26-3.22 (m, 2H), |
| | | 1.63-1.59 (m, 2H), 1.34 (dd, J = 26.9, 6.7 Hz, 6H), 0.84 (dd, |
| | | J = 7.8, 6.3 Hz, 6H); ESI MS (m/z): 389.1 [M + 1] |
| 498 | (cyclobutylmethyl)((2- | ¹H-NMR (400 MHz, DMSO-d₆) δ 9.19 (s, 1H), 8.95 (s, 1H), |
| | (5-fluoropyridin-3-yl)- | 8.62 (d, J = 2.8 Hz, 1H), 8.41 (dt, J = 10.1, 2.3 Hz, 1H), 7.55 (d, |
| | 2H-indazol-5- | J = 9.2 Hz, 1H), 7.14 (d, J = 1.2 Hz, 1H), 7.01 (dd, J = 9.3, 2.0 |
| | yl)imino)(isopropyl)-λ⁶- | Hz, 1H), 3.41 (dt, J = 19.4, 6.7 Hz, 3H), 2.79 (t, J = 8.1 Hz, 1H), |
| | sulfanone | 2.04 (t, J = 7.8 Hz, 2H), 1.84-1.72 (m, 4H), 1.39-1.29 (m, 6H) |
| | | ESI MS (m/z): 386.95 [M + 1]. |
| 499 | 1-((2-(5-fluoropyridin- | ¹H-NMR (400 MHz, DMSO-d₆) δ 9.19 (s, 1H), 8.96 (d, J = |
| | 3-yl)-2H-indazol-5- | 0.6 Hz, 1H), 8.63 (d, J = 2.4 Hz, 1H), 8.42 (dt, J = 10.1, 2.3 |
| | ylimino)hexahydro-λ⁶- | Hz, 1H), 7.58 (d, J = 9.2 Hz, 1H), 7.15 (d, J = 1.2 Hz, 1H), |
| | thiopyran 1-oxide | 7.04 (dd, J = 9.2, 2.1 Hz, 1H), 3.40-3.37 (m, 2H), 3.27-3.20 |
| | | (m, 2H), 1.93-1.88 (m, 4H), 1.66-1.54 (m, 2H); ESI MS (m/z): |
| | | 344.95 [M + 1]. |
| 500 | 1-((2-(pyridin-3-yl)-2H- | ¹H-NMR (400 MHz, DMSO-d₆) δ 9.33 (d, J = 2.4 Hz, 1H), 9.09 |
| | pyrazolo[3,4-c]pyridin- | (t, J = 1.1 Hz, 1H), 9.04 (d, J = 0.9 Hz, 1H), 8.68 (dd, J = 4.6, |
| | 5-yl)imino)tetrahydro- | 1.2 Hz, 1H), 8.50 (dq, J = 8.4, 1.3 Hz, 1H), 7.66 (dd, J = 8.3, |
| | 1H-λ⁶-thiophene 1- | 4.9 Hz, 1H), 6.85 (d, J = 1.5 Hz, 1H), 3.56-3.49 (m, 2H), 3.34 |
| | oxide | (d, J = 6.7 Hz, 1H), 3.30 (d, J = 6.7 Hz, 1H), 2.23-2.16 (m, 2H), |
| | | 2.14-2.07 (m, 2H); ESI MS (m/z): 314.1 [M + 1]. |
| 501 | 4-((2-(pyridin-3-yl)-2H- | ¹H-NMR (400 MHz, DMSO-d₆) δ 9.33 (d, J = 2.4 Hz, 1H), |
| | pyrazolo[3,4-c]pyridin- | 9.05 (s, 2H), 8.68 (dd, J = 4.7, 1.4 Hz, 1H), 8.49 (dq, J = 8.3, |
| | 5-yl)imino)-1,4 λ⁶- | 1.4 Hz, 1H), 7.66 (dd, J = 8.4, 4.7 Hz, 1H), 6.90 (d, J = 0.9 Hz, |
| | oxathiane 4-oxide | 1H), 4.07 (qd, J = 6.3, 3.2 Hz, 2H), 3.99-3.93 (m, 2H), 3.72 |
| | | (dt, J = 14.6, 3.1 Hz, 2H), 3.55-3.49 (m, 2H); ESI MS (m/z): |
| | | 330.05 [M + 1] |
| 502 | diethyl((2-(pyridin-3- | ¹H-NMR (400 MHz, DMSO-d₆) δ 9.32 (d, J = 2.4 Hz, 1H), 9.03 |
| | yl)-2H-pyrazolo[3,4- | (d, J = 13.0 Hz, 2H), 8.68 (d, J = 3.7 Hz, 1H), 8.49 (d, J = 8.3 |
| | c]pyridin-5-yl)imino)- | Hz, 1H), 7.65 (dd, J = 8.3, 4.6 Hz, 1H), 6.83 (d, J = 1.0 Hz, 1H), |
| | λ⁶-sulfanone | 3.52-3.41 (m, 4H), 1.27 (t, J = 7.3 Hz, 6H); ESI MS (m/z): 315.9 |
| | | [M + 1] |
| 503 | 1-((2-(pyrimidin-5-yl)- | ¹H-NMR (400 MHz, DMSO-d₆) δ 9.54 (s, 2H), 9.29 (d, J = 4.9 |
| | 2H-pyrazolo[3,4- | Hz, 1H), 9.12 (t, J = 1.1 Hz, 1H), 9.10 (d, J = 1.0 Hz, 1H), 6.87 |
| | c]pyridin-5- | (d, J = 1.5 Hz, 1H), 3.56-3.49 (m, 2H), 3.35 (d, J = 6.6 Hz, 1H), |
| | yl)imino)tetrahydro- | 3.29 (s, 1H), 2.24-2.16 (m, 2H), 2.14-2.05 (m, 2H) |
| | 1H-λ⁶-thiophene 1- | ESI MS (m/z): 314.95 [M + 1] |
| | oxide | |
| 504 | 4-((2-(pyridin-3-yl)-2H- | ¹H-NMR (400 MHz, DMSO-d₆) δ 9.26 (d, J = 2.4 Hz, 1H), |
| | indazol-5-yl)imino)-1,4 | 8.91 (d, J = 0.9 Hz, 1H), 8.60 (dd, J = 4.7, 1.4 Hz, 1H), 8.41 |
| | λ⁶-oxathiane 4-oxide | (dq, J = 8.3, 1.4 Hz, 1H), 7.60 (dd, J = 8.1, 5.0 Hz, 1H), 7.55 |
| | | (d, J = 9.2 Hz, 1H), 7.16 (d, J = 1.2 Hz, 1H), 7.00 (dd, J = 9.2, |
| | | 1.8 Hz, 1H), 4.1-4.05 (m, 2H), 3.98-3.90 (m, 2H), 3.52-3.45 |
| | | (m, 2H), 3.41-3.32 (m, 2H); ESI MS (m/z): 329 [M + 1] |
| 505 | isopropyl((2-(pyridin-3- | ¹H-NMR (400 MHz, DMSO-d₆) δ 9.27 (d, J = 2.4 Hz, 1H), 8.93 |
| | yl)-2H-indazol-5- | (d, J = 0.6 Hz, 1H), 8.60 (dd, J = 4.7, 1.4 Hz, 1H), 8.53 (dd, J = |
| | yl)imino)(pyridin-4- | 4.3, 1.5 Hz, 2H), 8.42 (dq, J = 8.4, 1.3 Hz, 1H), 7.61 (q, J = 4.3 |
| | ylmethyl)-λ⁶-sulfanone | Hz, 1H), 7.56 (d, J = 9.2 Hz, 1H), 7.40-7.38 (m, 2H), 7.19 (t, |
| | | J = 0.9 Hz, 1H), 6.99 (dd, J = 9.3, 2.0 Hz, 1H), 4.73 (q, J = 13.6 |
| | | Hz, 1H), 3.37 (t, J = 6.7 Hz, 1H), 2.53 (d, J = 2.4 Hz, 1H), 1.40 |
| | | (d, J = 6.7 Hz, 3H), 1.34 (d, J = 7.0 Hz, 3H) |
| | | ESI MS (m/z): 392.05 [M + 1]. |
| 506 | isopropyl(pyridin-2- | ¹H-NMR (400 MHz, DMSO-d₆) δ 9.28 (d, J = 2.4 Hz, 1H), 8.95 |
| | ylmethyl)((2-(pyridin- | (d, J = 0.9 Hz, 1H), 8.58 (ddd, J = 17.7, 4.6, 1.2 Hz, 2H), 8.42 |
| | 3-yl)-2H-indazol-5- | (t, J = 1.4 Hz, 1H), 7.76 (d, J = 1.8 Hz, 1H), 7.61 (q, J = 4.4 Hz, |
| | yl)imino)-λ⁶-sulfanone | 1H), 7.56 (d, J = 9.2 Hz, 1H), 7.36-7.30 (m, 2H), 7.24 (d, J = |
| | | 1.2 Hz, 1H), 6.95 (dd, J = 9.2, 2.1 Hz, 1H), 4.78 (dd, J = 31.5, |
| | | 14.1 Hz, 2H), 3.50 (s, 1H), 1.43 (d, J = 6.7 Hz, 3H), 1.38 (d, |
| | | J = 7.0 Hz, 3H); ESI MS (m/z): 392 [M + 1] |
| 507 | ethyl((2-(pyridin-3-yl)- | ¹H-NMR (400 MHz, DMSO-d₆) δ 9.20 (d, J = 2.1 Hz, 1H), |
| | 2H-indazol-5- | 8.87-8.86 (m, 3H), 8.58 (dd, J = 4.7, 1.4 Hz, 1H), 8.35 (dq, J = |
| | yl)imino)(pyridin-4-yl)- | 8.3, 1.4 Hz, 1H), 7.86 (dd, J = 4.3, 1.5 Hz, 2H), 7.60-7.54 (m, |
| | λ⁶-sulfanone | 2H), 7.05-7.02 (m, 2H), 3.63 (td, J = 7.6, 7.0 Hz, 2H), 1.20 (t, |
| | | J = 7.3 Hz, 3H); ESI MS (m/z): 364.05 [M + 1] |
| 508 | 4-((2-(pyrimidin-5-yl)- | ¹H-NMR (400 MHz, DMSO-d₆) δ 9.47 (s, 2H), 9.21 (s, 1H), |
| | 2H-indazol-5- | 8.97 (d, J = 0.6 Hz, 1H), 7.57 (d, J = 9.2 Hz, 1H), 7.19 (d, J = |
| | yl)imino)-1,4 λ⁶- | 1.2 Hz, 1H), 7.06 (dd, J = 9.2, 2.1 Hz, 1H), 4.1-4.02 (m, 2H), |
| | oxathiane 4-oxide | 3.98-3.88 (m, 2H), 3.52-3.45 (m, 2H), 3.44-3.34 (m, 2H). |
| | | ESI MS (m/z): 330 [M + 1]. |
| 509 | (3- | ¹H-NMR (400 MHz, DMSO-d₆) δ 9.47-9.41 (m, 2H), 9.18 (d, J = |
| | methoxyphenyl)(methyl)((2- | 10.1 Hz, 1H), 8.91 (s, 1H), 7.56-7.44 (m, 4H), 7.38-7.19 (m, |
| | (pyrimidin-5-yl)- | 1H), 7.09-7.00 (m, 2H), 3.79-3.76 (m, 3H), 3.42 (s, 3H) |
| | 2H-indazol-5- | ESI MS (m/z): 379.95 [M + 1] |
| | yl)imino)-λ⁶-sulfanone | |
| 510 | methyl(pyridin-2- | ¹H-NMR (400 MHz, DMSO-d₆) δ 9.28 (d, J = 2.4 Hz, 1H), 8.95 |
| | yl)((2-(pyrimidin-5-yl)- | (d, J = 0.9 Hz, 1H), 8.58 (ddd, J = 17.7, 4.6, 1.2 Hz, 1H), 8.42 |
| | 2H-indazol-5- | (t, J = 1.4 Hz, 1H), 7.76 (d, J = 1.8 Hz, 1H), 7.61 (q, J = 4.4 Hz, |
| | yl)imino)-λ⁶-sulfanone | 1H), 7.56 (d, J = 9.2 Hz, 1H), 7.36-7.30 (m, 2H), 7.24 (d, J = |
| | | 1.2 Hz, 1H), 6.95 (dd, J = 9.2, 2.1 Hz, 1H), 3.50 (s, 3H) |
| | | ESI MS (m/z): 350.9 [M + 1] |
| 511 | ethyl(pyridin-4-yl)((2- | ¹H-NMR (400 MHz, DMSO-d₆) δ 9.20 (d, J = 2.1 Hz, 1H), |
| | (pyrimidin-5-yl)-2H- | 8.87-8.86 (m, 2H), 8.58 (dd, J = 4.7, 1.4 Hz, 1H), 8.35 (dq, J = |
| | indazol-5-yl)imino)-λ⁶- | 8.3, 1.4 Hz, 1H), 7.86 (dd, J = 4.3, 1.5 Hz, 2H), 7.60-7.54 (m, |
| | sulfanone | 2H), 7.05-7.02 (m, 2H), 3.63 (td, J = 7.6, 7.0 Hz, 2H), 1.20 (t, |
| | | J = 7.3 Hz, 3H); ESI MS (m/z): 364.95 [M + 1] |
| 512 | (cyclopropylmethyl)(iso- | ¹H-NMR (400 MHz, DMSO-d₆) δ 9.47 (s, 2H), 9.21 (s, 1H), |
| | propyl)((2-(pyrimidin- | 8.97 (d, J = 0.6 Hz, 1H), 7.57 (d, J = 9.2 Hz, 1H), 7.19 (d, J = |
| | 5-yl)-2H-indazol-5- | 1.2 Hz, 1H), 7.06 (dd, J = 9.2, 2.1 Hz, 1H), 3.57 (t, J = 6.9 Hz, |
| | yl)imino)-λ⁶-sulfanone | 1H), 3.28 (d, J = 6.7 Hz, 2H), 1.39 (d, J = 6.7 Hz, 3H), 1.33 (d, |
| | | J = 7.0 Hz, 3H), 1.04 (s, 1H), 0.56-0.54 (m, 2H), 0.30 (dd, J = |
| | | 4.9, 3.1 Hz, 2H); ESI MS (m/z): 356 [M + 1]. |
| 513 | ((2-(5-fluoropyridin-3- | ¹H-NMR (400 MHz, DMSO-d₆) δ 9.13 (s, 1H), 8.89 (d, J = 0.9 |
| | yl)-2H-indazol-5- | Hz, 1H), 8.61 (d, J = 2.4 Hz, 1H), 8.35 (dt, J = 10.1, 2.3 Hz, |
| | yl)imino)(3- | 1H), 7.54-7.51 (m, 3H), 7.44 (t, J = 1.4 Hz, 1H), 7.21 (td, J = |
| | methoxyphenyl)(methyl)- | 4.7, 2.6 Hz, 1H), 7.04 (dd, J = 9.2, 2.1 Hz, 1H), 6.98 (q, J = 0.9 |
| | λ⁶-sulfanone | Hz, 1H), 3.80 (d, J = 5.5 Hz, 3H), 3.42 (s, 3H); ESI MS (m/z): |
| | | 396.95 [M + 1] |
| 514 | (2- | ¹H-NMR (400 MHz, DMSO-d₆) δ 9.32 (d, J = 2.4 Hz, 1H), 9.04 |
| | methoxyethyl)(methyl)((2- | (dd, J = 12.1, 0.8 Hz, 2H), 8.68 (dd, J = 4.7, 1.4 Hz, 1H), 8.49 |
| | (pyridin-3-yl)-2H- | (dq, J = 8.3, 1.4 Hz, 1H), 7.65 (dd, J = 8.3, 4.9 Hz, 1H), 6.84 (d, |
| | pyrazolo[3,4-c]pyridin- | J = 1.5 Hz, 1H), 3.86-3.74 (m, 4H), 3.28 (d, J = 1.5 Hz, 6H) |
| | 5-yl)imino)-λ⁶- | ESI MS (m/z): 331.9 [M + 1] |
| | sulfanone | |
| 515 | 1-((3-methyl-2- | ¹H-NMR (400 MHz, DMSO-d₆) δ 8.89 (d, J = 2.0 Hz, 1H), 8.70 |
| | (pyridin-3-yl)-2H- | (dd, J = 4.9, 1.5 Hz, 1H), 8.12 (dq, J = 8.1, 1.3 Hz, 1H), 7.66- |
| | indazol-5- | 7.62 (m, 1H), 7.48 (dd, J = 9.0, 0.7 Hz, 1H), 7.10 (d, J = 1.2 Hz, |
| | ylimino)hexahydro-λ⁶- | 1H), 7.02 (dd, J = 9.0, 2.0 Hz, 1H), 3.38-3.34 (m, 2H), 3.25- |
| | thiopyran 1-oxide | 3.18 (m, 2H), 2.59 (s, 3H), 1.91-1.87 (m, 4H), 1.65-1.54 (m, |
| | | 2H); ESI MS (m/z): 341.0 [M + 1] |
| 516 | 4-((2-(5-fluoropyridin- | ¹H-NMR (400 MHz, CHLOROFORM-D) δ 9.11 (s, 1H), 9.05- |
| | 3-yl)-2H-pyrazolo[3,4- | 8.98 (m, 1H), 8.58 (dd, J = 6.4, 2.4 Hz, 1H), 8.35 (d, J = 0.6 Hz, |
| | c]pyridin-5-yl)imino)- | 1H), 8.11 (dt, J = 9.0, 2.4 Hz, 1H), 7.01 (s, 1H), 4.27-4.15 (m, |
| | 1,4 λ⁶-oxathiane 4- | 4H), 3.77 (dt, J = 14.7, 2.8 Hz, 2H), 3.54-3.48 (m, 2H) |
| | oxide | ESI MS (m/z): 347.9 [M + 1] |
| 517 | ethyl((2-(5- | ¹H-NMR (400 MHz, DMSO-d₆) δ 8.83 (d, J = 1.5 Hz, 1H), 8.74 |
| | fluoropyridin-3-yl)-3- | (d, J = 2.7 Hz, 1H), 8.20 (dt, J = 9.5, 2.3 Hz, 1H), 7.46 (dd, J = |
| | methyl-2H-indazol-5- | 9.0, 0.7 Hz, 1H), 7.11 (q, J = 0.9 Hz, 1H), 7.04 (dd, J = 9.2, 2.1 |
| | yl)imino)(isopropyl)-λ⁶- | Hz, 1H), 3.56-3.49 (m, 2H), 3.33-3.24 (m, 2H), 2.62 (s, 3H), |
| | sulfanone | 1.37 (d, J = 6.8 Hz, 2H), 1.31 (d, J = 6.8 Hz, 3H), 1.24 (t, J = |
| | | 7.3 Hz, 3H); ESI MS (m/z): 360.95 [M + 1] |
| 518 | 1-((2-(5-fluoropyridin- | ¹H-NMR (400 MHz, CHLOROFORM-D) δ 8.73 (s, 1H), 8.59 |
| | 3-yl)-3-methyl-2H- | (d, J = 2.7 Hz, 1H), 7.78 (dt, J = 8.7, 2.3 Hz, 1H), 7.60 (dd, J = |
| | indazol-5- | 9.0, 0.7 Hz, 1H), 7.25 (d, J = 1.5 Hz, 1H), 7.15 (dd, J = 9.3, 2.0 |
| | yl)imino)tetrahydro- | Hz, 1H), 3.62-3.53 (m, 2H), 3.25-3.18 (m, 2H), 2.69 (s, 3H), |
| | 1H-λ⁶-thiophene 1- | 2.42-2.28 (m, 4H); ESI MS (m/z): 344.9 [M + 1]. |
| | oxide | |
| 519 | 1-((2-(5-fluoropyridin- | ¹H-NMR (400 MHz, DMSO-d₆) δ 8.82 (s, 1H), 8.75 (d, J = 2.4 |
| | 3-yl)-3-methyl-2H- | Hz, 1H), 8.20 (dt, J = 9.8, 2.3 Hz, 1H), 7.48 (d, J = 9.3 Hz, 1H), |
| | indazol-5- | 7.10 (d, J = 1.2 Hz, 1H), 7.03 (dd, J = 9.0, 2.0 Hz, 1H), 3.42- |
| | yl)imino)hexahydro-λ⁶- | 3.33 (m, 2H), 3.26-3.19 (m, 2H), 2.63 (s, 3H), 1.92-1.88 (m, |
| | thiopyran 1-oxide | 4H), 1.64-1.57 (m, 2H); ESI MS (m/z): 358.9 [M + 1] |
| 520 | diethyl((2-(5- | ¹H-NMR (400 MHz, DMSO-d₆) δ 8.83 (s, 1H), 8.75 (d, J = 2.7 |
| | fluoropyridin-3-yl)-3- | Hz, 1H), 8.20 (dt, J = 9.7, 2.3 Hz, 1H), 7.47 (dd, J = 9.2, 0.6 Hz, |
| | methyl-2H-indazol-5- | 1H), 7.10 (t, J = 1.0 Hz, 1H), 7.01 (dd, J = 9.2, 2.1 Hz, 1H), |
| | yl)imino)-λ⁶-sulfanone | 3.35-3.32 (m, 1H), 3.31-3.25 (m, 3H), 2.62 (s, 3H), 1.25 (t, J = |
| | | 7.5 Hz, 6H); ESI MS (m/z): 346.95 [M + 1] |
| 521 | 4-((3-methyl-2- | ¹H-NMR (400 MHz, DMSO-d₆) δ 8.89 (d, J = 2.0 Hz, 1H), 8.70 |
| | (pyridin-3-yl)-2H- | (dd, J = 4.6, 1.5 Hz, 1H), 8.12 (dq, J = 8.2, 1.3 Hz, 1H), 7.66- |
| | indazol-5-yl)imino)-1,4 | 7.59 (m, 1H), 7.50 (dd, J = 9.2, 0.6 Hz, 1H), 7.16 (t, J = 1.0 Hz, |
| | λ⁶-oxathiane 4-oxide | 1H), 7.08-7.03 (m, 1H), 4.10-4.02 (m, 2H), 3.98-3.92 (m, 2H), |
| | | 3.49-3.45 (m, 2H), 3.40-3.34 (m, 2H), 2.60 (s, 3H) |
| | | ESI MS (m/z): 342.95 [M + 1] |
| 522 | ((2-(5-fluoropyridin-3- | ¹H-NMR (400 MHz, DMSO-d₆) δ 9.19 (s, 1H), 9.07 (d, J = 0.7 |
| | yl)-2H-pyrazolo[4,3- | Hz, 1H), 8.63 (d, J = 2.4 Hz, 1H), 8.41 (dt, J = 10.1, 2.3 Hz, |
| | b]pyridin-5- | 1H), 7.95 (dd, J = 9.0, 0.7 Hz, 1H), 6.83 (d, J = 9.0 Hz, 1H), |
| | yl)imino)(isopropyl)(2- | 4.08-4.02 (m, 1H), 3.93-3.86 (m, 1H), 3.82-3.73 (m, 2H), 3.72- |
| | methoxyethyl)-λ⁶- | 3.63 (m, 1H), 3.25 (d, J = 12.2 Hz, 3H), 1.45-1.37 (m, 6H) |
| | sulfanone | ESI MS (m/z): 378.1 [M + 1] |
| 523 | (cyclopropylmethyl)((2- | ¹H-NMR (400 MHz, DMSO-d₆) δ 9.19 (s, 1H), 9.06 (d, J = 0.7 |
| | (5-fluoropyridin-3-yl)- | Hz, 1H), 8.63 (d, J = 2.4 Hz, 1H), 8.40 (dt, J = 10.2, 2.3 Hz, |
| | 2H-pyrazolo[4,3- | 1H), 7.95-7.93 (m, 1H), 6.82 (q, J = 4.6 Hz, 1H), 3.85-3.78 (m, |
| | b]pyridin-5- | 1H), 3.71 (dq, J = 25.5, 7.2 Hz, 2H), 1.39 (dd, J = 8.1, 6.8 Hz, |
| | yl)imino)(isopropyl)-λ⁶- | 6H), 1.10-1.04 (m, 1H), 0.64-0.54 (m, 2H), 0.41-0.28 (m, 2H) |
| | sulfanone | ESI MS (m/z): 374.00 [M + 1] |
| 524 | 1-((2-(5-fluoropyridin- | ¹H-NMR (400 MHz, DMSO-d₆) δ 9.21 (s, 1H), 9.10 (d, J = 1.0 |
| | 3-yl)-2H-pyrazolo[4,3- | Hz, 1H), 8.63 (d, J = 2.2 Hz, 1H), 8.43 (dt, J = 10.2, 2.3 Hz, |
| | b]pyridin-5- | 1H), 7.98 (dd, J = 9.3, 0.7 Hz, 1H), 6.84 (d, J = 9.0 Hz, 1H), |
| | yl)imino)tetrahydro- | 3.67-3.60 (m, 2H), 3.36 (q, J = 6.8 Hz, 2H), 2.26-2.19 (m, 2H), |
| | 1H-λ⁶-thiophene 1- | 2.15-2.06 (m, 2H); ESI MS (m/z): 331.85 [M + 1] |
| | oxide | |
| 525 | 1-((2-(5-fluoropyridin- | ¹H-NMR (400 MHz, DMSO-d₆) δ 9.19 (s, 1H), 9.04 (d, J = 1.0 |
| | 3-yl)-2H-pyrazolo[4,3- | Hz, 1H), 8.63 (d, J = 2.4 Hz, 1H), 8.40 (dt, J = 10.0, 2.3 Hz, |
| | b]pyridin-5- | 1H), 7.96 (dd, J = 9.2, 0.9 Hz, 1H), 6.83 (d, J = 9.3 Hz, 1H), |
| | yl)imino)hexahydro-λ⁶- | 3.74 (d, J = 8.1 Hz, 2H), 3.50-3.43 (m, 2H), 1.99-1.86 (m, 4H), |
| | thiopyran 1-oxide | 1.63-1.58 (m, 2H); ESI MS (m/z): 345.95 [M + 1] |
| 526 | 4-((2-(5-fluoropyridin- | ¹H-NMR (400 MHz, DMSO-d₆) δ 9.20 (s, 1H), 9.08 (d, J = 1.0 |
| | 3-yl)-2H-pyrazolo[4,3- | Hz, 1H), 8.64 (d, J = 2.2 Hz, 1H), 8.41 (dt, J = 10.1, 2.4 Hz, |
| | b]pyridin-5-yl)imino)- | 1H), 8.00 (dd, J = 9.2, 0.9 Hz, 1H), 6.87 (d, J = 9.3 Hz, 1H), |
| | 1,4 λ⁶-oxathiane 4- | 4.11 (ddd, J = 12.6, 5.7, 3.3 Hz, 2H), 3.96-3.86 (m, 4H), 3.60- |
| | oxide | 3.54 (m, 2H); ESI MS (m/z): 347.95 [M + 1] |
| 527 | ((2-(5-fluoropyridin-3- | ¹H-NMR (400 MHz, DMSO-d₆) δ 9.23 (s, 1H), 9.17 (d, J = 1.0 |
| | yl)-2H-pyrazolo[4,3- | Hz, 1H), 8.65 (d, J = 2.7 Hz, 1H), 8.59-8.58 (m, 1H), 8.45 (dt, |
| | b]pyridin-5- | J = 10.2, 2.3 Hz, 1H), 7.96 (dd, J = 9.2, 0.9 Hz, 1H), 7.76 (td, |
| | yl)imino)(isopropyl)(pyridin- | J = 7.7, 1.8 Hz, 1H), 7.38-7.33 (m, 2H), 6.77 (d, J = 9.3 Hz, 1H), |
| | 2-ylmethyl)-λ⁶- | 5.26 (s, 2H), 3.66 (t, J = 6.8 Hz, 1H), 1.43 (dd, J = 15.6, 6.8 Hz, |
| | sulfanone | 6H); ESI MS (m/z): 410.95 [M + 1] |
| 528 | ((2-(5-fluoropyridin-3- | ¹H-NMR (400 MHz, DMSO-d₆) δ 9.21 (s, 1H), 9.12 (d, J = 0.7 |
| | yl)-2H-pyrazolo[4,3- | Hz, 1H), 8.64 (d, J = 2.2 Hz, 1H), 8.56-8.54 (m, 2H), 8.43 (dt, |
| | b]pyridin-5- | J = 10.0, 2.3 Hz, 1H), 7.98 (dd, J = 9.2, 0.9 Hz, 1H), 7.82 (d, |
| | yl)imino)(isopropyl)(pyridin- | J = 7.8 Hz, 1H), 7.42 (dd, J = 7.5, 4.5 Hz, 1H), 6.81 (d, J = 9.0 |
| | 3-ylmethyl)-λ⁶- | Hz, 1H), 5.13 (d, J = 13.7 Hz, 1H), 5.05 (d, J = 13.9 Hz, 1H), |
| | sulfanone | 3.77 (t, J = 6.8 Hz, 1H), 1.41 (dd, J = 18.6, 6.8 Hz, 6H) |
| | | ESI MS (m/z): 411.0 [M + 1] |
| 529 | ((2-(5-fluoropyridin-3- | ¹H-NMR (400 MHz, DMSO-d₆) δ 9.22 (s, 1H), 9.13 (d, J = 1.0 |
| | yl)-2H-pyrazolo[4,3- | Hz, 1H), 8.64 (d, J = 2.4 Hz, 1H), 8.58 (d, J = 6.1 Hz, 2H), 8.46- |
| | b]pyridin-5- | 8.42 (m, 1H), 7.99 (dd, J = 9.2, 0.9 Hz, 1H), 7.43-7.42 (m, 2H), |
| | yl)imino)(isopropyl)(pyridin- | 6.83 (d, J = 9.0 Hz, 1H), 5.15 (d, J = 13.4 Hz, 1H), 5.08 (d, J = |
| | 4-ylmethyl)-λ⁶- | 13.4 Hz, 1H), 3.69 (t, J = 6.8 Hz, 1H), 1.40 (dd, J = 14.8, 6.7 |
| | sulfanone | Hz, 6H); ESI MS (m/z): 411.00 [M + 1] |
| 530 | ((2-(5-fluoropyridin-3- | ¹H-NMR (400 MHz, DMSO-d₆) δ 9.16 (s, 1H), 8.98 (d, J = 1.0 |
| | yl)-2H-pyrazolo[4,3- | Hz, 1H), 8.61 (d, J = 2.4 Hz, 1H), 8.39 (dt, J = 10.2, 2.3 Hz, |
| | b]pyridin-5- | 1H), 7.98 (dd, J = 9.2, 0.9 Hz, 1H), 7.58-7.52 (m, 2H), 7.51 (t, |
| | yl)imino)(3- | J = 1.5 Hz, 1H), 7.25 (td, J = 4.7, 2.4 Hz, 1H), 6.93 (d, J = 9.3 |
| | methoxyphenyl)(methyl)- | Hz, 1H), 3.83-3.81 (m, 3H), 3.52 (s, 3H); ESI MS (m/z): 397.95 |
| | λ⁶-sulfanone | [M + 1] |
| 531 | ((2-(5-fluoropyridin-3- | ¹H-NMR (400 MHz, DMSO-d₆) δ 9.19 (s, 1H), 9.05 (d, J = 0.7 |
| | yl)-2H-pyrazolo[4,3- | Hz, 1H), 8.63 (d, J = 2.7 Hz, 1H), 8.41 (dt, J = 10.0, 2.3 Hz, |
| | b]pyridin-5- | 1H), 7.94 (dd, J = 9.2, 0.9 Hz, 1H), 6.81 (d, J = 9.3 Hz, 1H), |
| | yl)imino)(isopropyl)(propyl)- | 3.96-3.89 (m, 1H), 3.64-3.49 (m, 2H), 1.84-1.69 (m, 2H), 1.37 |
| | λ⁶-sulfanone | (dd, J = 8.2, 7.0 Hz, 6H), 0.99 (t, J = 7.5 Hz, 3H) |
| | | ESI MS (m/z): 362.00 [M + 1] |
| 532 | (cyclobutylmethyl)((2- | ¹H-NMR (400 MHz, DMSO-d₆) δ 9.20 (s, 1H), 9.06 (s, 1H), |
| | (5-fluoropyridin-3-yl)- | 8.63 (d, J = 2.7 Hz, 1H), 8.41 (d, J = 10.3 Hz, 1H), 7.93 (d, J = |
| | 2H-pyrazolo[4,3- | 9.3 Hz, 1H), 6.79 (d, J = 9.0 Hz, 1H), 3.81-3.71 (m, 3H), 2.73- |
| | b]pyridin-5- | 2.94 (1H), 1.96-2.20 (2H), 1.87-1.85 (m, 4H), 1.35 (dd, J = 6.8, |
| | yl)imino)(isopropyl)-λ⁶- | 5.1 Hz, 6H); ESI MS (m/z): 388.00 [M + 1] |
| | sulfanone | |
| 533 | 4-((2-(pyrimidin-5-yl)- | ¹H-NMR (400 MHz, DMSO-d₆) δ 9.54 (s, 2H), 9.30 (s, 1H), |
| | 2H-pyrazolo[3,4- | 9.11 (d, J = 0.9 Hz, 1H), 9.09 (t, J = 1.2 Hz, 1H), 6.92 (d, J = |
| | c]pyridin-5-yl)imino)- | 1.5 Hz, 1H), 4.08 (qd, J = 6.3, 3.2 Hz, 2H), 4.00-3.94 (m, 2H), |
| | 1,4 λ⁶-oxathiane 4- | 3.72 (dt, J = 14.8, 3.1 Hz, 2H), 3.56-3.50 (m, 2H) |
| | oxide | ESI MS (m/z): 330.9[M + 1]. |
| 534 | ((2-(5-fluoropyridin-3- | ¹H-NMR (400 MHz, DMSO-d₆) δ 8.83 (s, 1H), 8.75 (d, J = |
| | yl)-3-methyl-2H- | 2.7 Hz, 1H), 8.21 (dt, J = 9.8, 2.3 Hz, 1H), 7.47 (d, J = 8.6 Hz, |
| | indazol-5- | 1H), 7.11 (d, J = 1.2 Hz, 1H), 7.01 (dd, J = 9.0, 2.0 Hz, 1H), |
| | yl)imino)(isopropyl)(methyl)- | 3.54-3.47 (m, 1H), 3.01 (s, 3H), 2.63 (s, 3H), 1.40 (d, J = 6.8 |
| | λ⁶-sulfanone | Hz, 3H), 1.35 (d, J = 6.8 Hz, 3H); ESI MS (m/z): 346.90 |
| | | [M + 1]. |
| 535 | ((2-(5-fluoropyridin-3- | ¹H-NMR (400 MHz, DMSO-d₆) δ 8.83 (t, J = 1.3 Hz, 1H), |
| | yl)-3-methyl-2H- | 8.75 (d, J = 2.7 Hz, 1H), 8.21 (dt, J = 9.5, 2.3 Hz, 1H), 7.47 |
| | indazol-5- | (dd, J = 9.0, 0.7 Hz, 1H), 7.09 (q, J = 0.9 Hz, 1H), 6.98 (dd, |
| | yl)imino)(methyl)(propyl)- | J = 9.0, 2.0 Hz, 1H), 3.40-3.33 (m, 2H), 3.12 (s, 3H), 2.64 (s, |
| | λ⁶-sulfanone | 3H), 1.83-1.74 (m, 2H), 0.98 (t, J = 7.3 Hz, 3H); ESI MS |
| | | (m/z): 346.90[M + 1]. |
| 536 | ((2-(5-fluoropyridin-3- | ¹H-NMR (400 MHz, DMSO-d₆) δ 8.83 (d, J = 1.2 Hz, 1H), |
| | yl)-3-methyl-2H- | 8.75 (d, J = 2.7 Hz, 1H), 8.21 (dt, J = 9.6, 2.3 Hz, 1H), 7.48 |
| | indazol-5- | (dd, J = 9.2, 0.6 Hz, 1H), 7.09 (q, J = 0.9 Hz, 1H), 6.97 (dd, |
| | yl)imino)dimethyl-λ⁶- | J = 9.0, 2.0 Hz, 1H), 3.22 (s, 6H), 2.63 (s, 3H); ESI MS (m/z): |
| | sulfanone | 318.90[M + 1]. |
| 537 | tert-butyl(methyl)((2- | ¹H-NMR (400 MHz, DMSO-d₆) δ 9.28 (d, J = 2.8 Hz, 1H), 9.05 |
| | (pyridin-3-yl)-2H- | (d, J = 0.6 Hz, 1H), 8.60 (dd, J = 4.6, 1.2 Hz, 1H), 8.42 (dq, J = |
| | pyrazolo[4,3-b]pyridin- | 8.3, 1.3 Hz, 1H), 7.95-7.92 (m, 1H), 7.61 (dd, J = 8.4, 4.7 Hz, |
| | 5-yl)imino)-λ⁶- | 1H), 6.78 (d, J = 9.2 Hz, 1H), 3.45 (s, 3H), 1.44 (d, J = 14.4 Hz, |
| | sulfanone | 9H); ESI MS (m/z): 330.1 [M + 1]. |
| 538 | ((6-chloropyridin-3- | ¹H-NMR (400 MHz, DMSO-d₆) δ 9.29 (d, J = 2.4 Hz, 1H), 9.06 |
| | yl)methyl)(isopropyl)((2- | (d, J = 0.9 Hz, 1H), 8.61 (dd, J = 4.7, 1.4 Hz, 1H), 8.45-8.41 (m, |
| | (pyridin-3-yl)-2H- | 2H), 7.99 (dd, J = 9.2, 0.6 Hz, 1H), 7.91 (dd, J = 8.3, 2.4 Hz, |
| | pyrazolo[4,3-b]pyridin- | 1H), 7.63-7.60 (m, 1H), 7.58 (d, J = 8.3 Hz, 1H), 6.80 (d, J = |
| | 5-yl)imino)-λ⁶- | 9.5 Hz, 1H), 5.11 (d, J = 13.8 Hz, 1H), 5.03 (d, J = 13.8 Hz, |
| | sulfanone | 1H), 3.85-3.79 (m, 1H), 1.41 (dd, J = 12.4, 6.9 Hz, 6H) |
| | | ESI MS (m/z): 427 [M + 1]. |
| 539 | ((2-(5-fluoropyridin-3- | ¹H-NMR (400 MHz, DMSO-d₆) δ 9.20 (s, 1H), 8.96 (d, J = 1.0 |
| | yl)-2H-indazol-5- | Hz, 1H), 8.63 (d, J = 2.4 Hz, 1H), 8.53 (dd, J = 4.5, 1.6 Hz, 2H), |
| | yl)imino)(isopropyl)(pyridin- | 8.42 (dt, J = 10.0, 2.3 Hz, 1H), 7.55 (d, J = 9.3 Hz, 1H), 7.39 |
| | 4-ylmethyl)-λ⁶- | (dd, J = 4.5, 1.6 Hz, 2H), 7.17 (q, J = 0.9 Hz, 1H), 7.00 (dd, J = |
| | sulfanone | 9.3, 2.0 Hz, 1H), 4.73 (dd, J = 26.2, 13.7 Hz, 2H), 3.40-3.33 (m, |
| | | 1H), 1.40 (d, J = 6.6 Hz, 3H), 1.34 (d, J = 6.8 Hz, 3H) |
| | | ESI MS (m/z): 410 [M + 1]. |
| 540 | ((2-(5-fluoropyridin-3- | ¹H-NMR (400 MHz, DMSO-d₆) δ 9.21 (s, 1H), 8.99 (s, 1H), |
| | yl)-2H-indazol-5- | 8.63 (d, J = 2.4 Hz, 1H), 8.56 (d, J = 4.2 Hz, 1H), 8.43 (dt, J = |
| | yl)imino)(isopropyl)(pyridin- | 10.3, 2.3 Hz, 1H), 7.76 (td, J = 7.7, 1.8 Hz, 1H), 7.56 (d, J = 9.3 |
| | 2-ylmethyl)-λ⁶- | Hz, 1H), 7.36-7.30 (m, 2H), 7.22 (d, J = 1.2 Hz, 1H), 6.96 (dd, |
| | sulfanone | J = 9.2, 2.1 Hz, 1H), 4.79 (dd, J = 31.8, 13.9 Hz, 2H), 3.50 (t, |
| | | J = 6.8 Hz, 1H), 1.43 (d, J = 6.8 Hz, 3H), 1.38 (d, J = 6.8 Hz, 3H) |
| | | ESI MS (m/z): 410.05 [M + 1]. |
| 541 | methyl(pyridin-2- | ¹H-NMR (400 MHz, DMSO-d₆) δ 9.21 (d, J = 2.4 Hz, 1H), 8.86 |
| | yl)((2-(pyridin-3-yl)- | (d, J = 0.7 Hz, 1H), 8.76 (dq, J = 4.7, 0.8 Hz, 1H), 8.58 (dd, J = |
| | 2H-indazol-5- | 4.6, 1.5 Hz, 1H), 8.36 (dq, J = 8.4, 1.3 Hz, 1H), 8.13-8.06 (m, |
| | yl)imino)-λ⁶-sulfanone | 2H), 7.66 (ddd, J = 7.3, 4.6, 1.5 Hz, 1H), 7.58 (dd, J = 8.7, 5.0 |
| | | Hz, 1H), 7.50 (d, J = 9.0 Hz, 1H), 7.04 (t, J = 1.0 Hz, 1H), 6.95 |
| | | (dd, J = 9.2, 2.1 Hz, 1H), 3.47 (s, 3H) |
| | | ESI MS (m/z): 349.9 [M + 1]. |
| 542 | isopropyl((2-(pyridin-3- | ¹H-NMR (400 MHz, DMSO-d₆) δ 9.29 (d, J = 2.4 Hz, 1H), 9.09 |
| | yl)-2H-pyrazolo[4,3- | (d, J = 0.9 Hz, 1H), 8.61 (dd, J = 4.7, 1.4 Hz, 1H), 8.57 (dd, J = |
| | b]pyridin-5- | 4.6, 1.5 Hz, 2H), 8.43 (dq, J = 8.4, 1.4 Hz, 1H), 7.99 (dd, J = |
| | yl)imino)(pyridin-4- | 9.2, 0.9 Hz, 1H), 7.63-7.60 (m, 1H), 7.42 (dd, J = 4.3, 1.5 Hz, |
| | ylmethyl)-λ⁶-sulfanone | 2H), 6.81 (d, J = 9.2 Hz, 1H), 5.11 (dd, J = 29.5, 13.6 Hz, 2H), |
| | | 3.73-3.66 (m, 1H), 1.40 (dd, J = 15.1, 6.9 Hz, 6H) |
| | | ESI MS (m/z): 392.95 [M + 1]. |
| 543 | ((2-(5-fluoropyridin-3- | ¹H-NMR (400 MHz, DMSO-d₆) δ 9.14 (s, 1H), 8.89 (d, J = 0.7 |
| | yl)-2H-indazol-5- | Hz, 1H), 8.76 (dq, J = 4.6, 0.8 Hz, 1H), 8.61 (d, J = 2.4 Hz, 1H), |
| | yl)imino)(propyl)(pyridin- | 8.36 (dt, J = 10.2, 2.3 Hz, 1H), 8.14-8.06 (m, 2H), 7.65 (ddd, J = |
| | 2-yl)-λ⁶-sulfanone | 7.3, 4.6, 1.5 Hz, 1H), 7.49 (d, J = 9.0 Hz, 1H), 7.01 (q, J = 1.0 |
| | | Hz, 1H), 6.98 (dd, J = 9.0, 2.0 Hz, 1H), 3.66-3.60 (m, 2H), 1.80- |
| | | 1.71 (m, 1H), 1.66-1.56 (m, 1H), 0.94 (t, J = 7.5 Hz, 3H) |
| | | ESI MS (m/z): 396 [M + 1]. |
| 544 | isopropyl(pyridin-2- | ¹H-NMR (400 MHz, DMSO-d₆) δ 9.30 (d, J = 2.4 Hz, 1H), 9.13 |
| | ylmethyl)((2-(pyridin- | (d, J = 0.9 Hz, 1H), 8.61 (dd, J = 4.6, 1.2 Hz, 1H), 8.58 (td, J = |
| | 3-yl)-2H-pyrazolo[4,3- | 2.9, 1.9 Hz, 1H), 8.44 (dq, J = 8.5, 1.4 Hz, 1H), 7.96 (dd, J = |
| | b]pyridin-5-yl)imino)- | 9.2, 0.6 Hz, 1H), 7.76 (td, J = 7.8, 1.8 Hz, 1H), 7.64-7.60 (m, |
| | λ⁶-sulfanone | 1H), 7.35 (dd, J = 7.6, 4.9 Hz, 2H), 6.76 (d, J = 9.2 Hz, 1H), |
| | | 5.24 (d, J = 14.4 Hz, 2H), 3.69-3.62 (m, 1H), 1.47-1.40 (m, 6H) |
| | | ESI MS (m/z): 393.05 [M + 1]. |
| 545 | ((2-(5-fluoropyridin-3- | ¹H-NMR (400 MHz, DMSO-d₆) δ 9.14 (s, 1H), 8.90 (d, J = 1.0 |
| | yl)-2H-indazol-5- | Hz, 1H), 8.76 (dq, J = 4.7, 0.8 Hz, 1H), 8.61 (d, J = 2.4 Hz, 1H), |
| | yl)imino)(methyl)(pyridin- | 8.37 (dt, J = 10.2, 2.3 Hz, 1H), 8.14-8.06 (m, 2H), 7.66 (ddd, J = |
| | 2-yl)-λ⁶-sulfanone | 7.3, 4.6, 1.5 Hz, 1H), 7.50 (d, J = 9.0 Hz, 1H), 7.02 (q, J = 0.9 |
| | | Hz, 1H), 6.96 (dd, J = 9.2, 2.1 Hz, 1H), 3.47 (s, 3H) |
| | | ESI MS (m/z): 367.95 [M + 1]. |
| 546 | (3- | ¹H-NMR (400 MHz, DMSO-d₆) δ 9.20 (d, J = 2.4 Hz, 1H), 8.85 |
| | methoxyphenyl)(methyl)((2- | (d, J = 0.6 Hz, 1H), 8.58 (dd, J = 4.6, 1.5 Hz, 1H), 8.35 (dq, J = |
| | (pyridin-3-yl)-2H- | 8.3, 1.3 Hz, 1H), 7.58 (q, J = 4.3 Hz, 1H), 7.54-7.49 (m, 3H), |
| | indazol-5-yl)imino)-λ⁶- | 7.44 (s, 1H), 7.21 (td, J = 4.6, 2.9 Hz, 1H), 7.04-7.00 (m, 2H), |
| | sulfanone | 3.81-3.74 (m, 3H), 3.40 (d, J = 9.5 Hz, 3H) |
| | | ESI MS (m/z): 378.9 [M + 1]. |
| 547 | isopentyl(isopropyl)((2- | ¹H-NMR (400 MHz, DMSO-d₆) δ 9.47 (s, 2H), 9.21 (s, 1H), |
| | (pyrimidin-5-yl)-2H- | 9.06 (s, 1H), 7.96 (d, J = 9.2 Hz, 1H), 6.82 (d, J = 9.2 Hz, 1H), |
| | pyrazolo[4,3-b]pyridin- | 3.93-3.86 (m, 1H), 3.65-3.60 (m, 2H), 1.73-1.62 (m, 2H), 1.59- |
| | 5-yl)imino)-λ⁶- | 1.51 (m, 1H), 1.39-1.34 (m, 6H), 0.87 (t, J = 6.0 Hz, 6H) |
| | sulfanone | ESI MS (m/z): 373.00 [M + 1]. |
| 548 | isopropyl(2- | ¹H-NMR (400 MHz, DMSO-d₆) δ 9.49-9.45 (m, 2H), 9.21 (s, |
| | methoxyethyl)((2- | 1H), 9.08 (d, J = 0.6 Hz, 1H), 7.97 (dd, J = 9.3, 0.8 Hz, 1H), |
| | (pyrimidin-5-yl)-2H- | 6.84 (d, J = 9.2 Hz, 1H), 4.08-4.02 (m, 1H), 3.93-3.86 (m, 1H), |
| | pyrazolo[4,3-b]pyridin- | 3.82-3.65 (m, 3H), 3.26 (s, 3H), 1.39 (dd, J = 8.6, 7.0 Hz, 6H) |
| | 5-yl)imino)-λ⁶- | ESI MS (m/z): 361.05[M + 1]. |
| | sulfanone | |
| 549 | ((4-chloro-2-(pyridin-3- | ¹H-NMR (400 MHz, DMSO-d₆) δ 9.33 (d, J = 2.4 Hz, 1H), 9.06 |
| | yl)-2H-indazol-5- | (d, J = 0.9 Hz, 1H), 8.63 (dd, J = 4.6, 1.5 Hz, 1H), 8.49 (dq, J = |
| | yl)imino)diethyl-λ⁶- | 8.3, 1.4 Hz, 1H), 7.62 (qd, J = 4.3, 0.6 Hz, 1H), 7.57 (dd, J = |
| | sulfanone | 8.9, 0.9 Hz, 1H), 7.36 (d, J = 8.9 Hz, 1H), 3.35 (d, J = 7.0 Hz, |
| | | 1H), 3.30-3.25 (m, 3H), 1.29 (t, J = 7.3 Hz, 6H) |
| | | ESI MS (m/z): 348.85[M + 1]. |
| 550 | ((4-chloro-2-(pyridin-3- | ¹H-NMR (400 MHz, DMSO-d₆) δ 9.33 (d, J = 2.1 Hz, 1H), |
| | yl)-2H-indazol-5- | 9.04 (d, J = 0.9 Hz, 1H), 8.62 (dd, J = 4.6, 1.2 Hz, 1H), 8.49 |
| | yl)imino)(ethyl)(isopropyl)- | (dq, J = 8.4, 1.4 Hz, 1H), 7.61 (ddd, J = 8.3, 4.9, 0.6 Hz, 1H), |
| | λ⁶-sulfanone | 7.55 (dd, J = 9.2, 0.9 Hz, 1H), 7.39-7.36 (m, 1H), 3.56-3.49 |
| | | (m, 1H), 3.28-3.24 (m, 2H), 1.40 (d, J = 6.7 Hz, 3H), 1.32 (d, |
| | | J = 7.0 Hz, 3H), 1.29-1.22 (m, 3H); ESI MS (m/z): 362.90 |
| | | M + 1]. |
| 551 | 1-((4-chloro-2- | ¹H-NMR (400 MHz, CHLOROFORM-D) δ 9.36-9.34 (m, |
| | (pyrimidin-5-yl)-2H- | 2H), 9.26 (s, 1H), 8.43 (d, J = 0.7 Hz, 1H), 7.61 (dd, J = 9.0, |
| | indazol-5- | 1.0 Hz, 1H), 7.44 (d, J = 9.3 Hz, 1H), 3.60-3.54 (m, 2H), 3.24- |
| | yl)imino)tetrahydro- | 3.19 (m, 2H), 2.43-2.37 (m, 4H); ESI MS (m/z): 347.90 |
| | 1H-λ⁶-thiophene 1- | [M + 1]. |
| | oxide | |
| 552 | ((4-chloro-2-(5- | ¹H-NMR (400 MHz, DMSO-d₆) δ 9.25 (d, J = 9.0 Hz, 1H), 9.11 |
| | fluoropyridin-3-yl)-2H- | (s, 1H), 8.66 (d, J = 2.4 Hz, 1H), 8.52 (dt, J = 10.1, 2.3 Hz, 1H), |
| | indazol-5- | 7.57 (d, J = 8.3 Hz, 1H), 7.38 (d, J = 9.0 Hz, 1H), 3.35 (t, J = |
| | yl)imino)diethyl-λ⁶- | 7.1 Hz, 2H), 3.28 (t, J = 7.2 Hz, 2H), 1.29 (t, J = 7.5 Hz, 6H) |
| | sulfanone | ESI MS (m/z): 366.85[M + 1] |
| 553 | ((4-chloro-2-(5- | ¹H-NMR (400 MHz, CHLOROFORM-D) δ 9.02 (d, J = 1.7 Hz, |
| | fluoropyridin-3-yl)-2H- | 1H), 8.51 (d, J = 2.4 Hz, 1H), 8.41-8.38 (m, 1H), 8.10 (dt, J = |
| | indazol-5- | 9.0, 2.3 Hz, 1H), 7.54-7.46 (m, 2H), 3.53-3.46 (m, 1H), 3.31 |
| | yl)imino)(ethyl)(isopropyl)- | (td, J = 14.4, 7.2 Hz, 1H), 3.18 (td, J = 14.4, 7.2 Hz, 1H), 1.56 |
| | λ⁶-sulfanone | (t, J = 7.0 Hz, 3H), 1.50 (d, J = 6.8 Hz, 3H), 1.44 (t, J = 7.5 Hz, |
| | | 3H); ESI MS (m/z): 380.90[M + 1]. |

Note:
For certain substrates, better outcomes were observed when cesium carbonate (Cs₂CO₃) or tribasic potassium phosphate (K₃PO₄) were used instead of sodium tert-butoxide.

### Example 2: Synthesis of N-(dimethyl(oxo)-λ⁶-sulfaneylidene)-2-(pyridin-3-yl)-2H-indazole-4-carboxamide 92

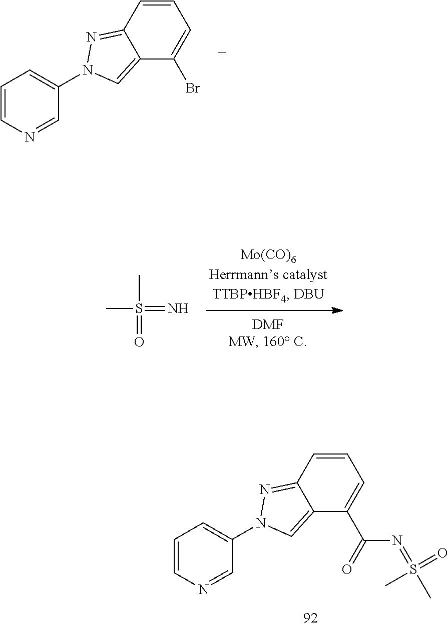

A solution of 4-bromo-2-(pyridin-3-yl)-2H-indazole (0.3 g, 1.094 mmol), iminodimethyl-λ⁶-sulfanone (0.306 g, 3.28 mmol) in dimethyl formamide (DMF) (5 mL) was treated with molybdenum hexacarbonyl (Mo(CO)₆) (0.144 g, 0.547 mmol), tri-tert-butylphosphine tetrafluoroborate (TTBP·HBF₄) (0.016 g, 0.055 mmol), trans-Di(μ-acetato)bis[o-(di-o-tolylphosphino)benzyl]dipalladium(II) (Herrmann's catalyst) (0.026 g, 0.027 mmol) and 1,8-diazabicyclo[5.4.0]undec-7-ene (DBU) (0.330 ml, 2.189 mmol), and irradiated in a microwave device (MW) at 160° C. for 1 h. After completion of the reaction, the reaction mixture was quenched by the addition of water (10 mL) and extracted with ethyl acetate (2×25 mL). The combined organic layers were dried over anhydrous sodium sulphate and concentrated under reduced pressure to obtain an oily residue. This residue was purified by reverse phase preparative-HPLC to obtain the desired N-(dimethyl(oxo)-λ⁶-sulfaneylidene)-2-(pyridin-3-yl)-2H-indazole-4-carboxamide 92 (220 mg, 0.700 mmol, 63.9% yield).

TABLE 2
Representative compounds of the present disclosure were prepared
according to the method as described in the Example 2.

| Comp | | |
|---|---|---|
| no. | Compound name | Analytical Data |
| 92 | N-(dimethyl(oxo)-λ⁶- | ¹H-NMR (400 MHz, DMSO-d₆) δ 9.37 (d, J = 1.0 Hz, 2H), |
| | sulfaneylidene)-2- | 8.67 (dd, J = 4.6, 1.5 Hz, 1H), 8.55 (dq, J = 8.3, 1.4 Hz, 1H), |
| | (pyridin-3-yl)-2H- | 7.95 (dt, J = 8.6, 0.9 Hz, 1H), 7.88 (dd, J = 7.1, 0.7 Hz, 1H), |
| | indazole-4-carboxamide | 7.64 (ddd, J = 8.4, 4.8, 0.7 Hz, 1H), 7.43 (dd, J = 8.6, 7.0 Hz, |
| | | 1H), 3.55 (s, 6H); ESI MS (m/z): 315 [M + 1]. |
| 225 | N-(butyl(ethyl)(oxo)-λ⁶- | ¹H-NMR (400 MHz, DMSO-d₆) δ 9.54 (s, 2H), 9.45 (d, J = |
| | sulfaneylidene)-2- | 0.7 Hz, 1H), 9.28 (s, 1H), 8.55 (t, J = 1.1 Hz, 1H), 7.95 (dd, |
| | (pyrimidin-5-yl)-2H- | J = 9.2, 1.6 Hz, 1H), 7.77-7.74 (m, 1H), 3.68-3.52 (m, 4H), |
| | indazole-5-carboxamide | 1.83-1.72 (m, 2H), 1.46 (td, J = 14.7, 7.4 Hz, 2H), 1.34 (t, J = |
| | | 7.3 Hz, 3H), 0.93 (t, J = 7.3 Hz, 3H); ESI MS(m/z): 372.1 |
| | | [M + 1]. |
| 226 | N- | ¹H-NMR (400 MHz, DMSO-d₆) δ 9.54 (s, 2H), 9.45 (d, J = |
| | (cyclohexyl(methyl)(oxo)- | 0.7 Hz, 1H), 9.28 (s, 1H), 8.55 (s, 1H), 7.95 (dd, J = 9.2, 1.6 |
| | 26-sulfaneylidene)-2- | Hz, 1H), 7.76 (d, J = 9.3 Hz, 1H), 3.58-3.52 (m, 1H), 3.44 (s, |
| | (pyrimidin-5-yl)-2H- | 3H), 2.25-2.18 (m, 2H), 1.90 (d, J = 12.5 Hz, 2H), 1.69 (d, |
| | indazole-5-carboxamide | J = 13.0 Hz, 1H), 1.60-1.54 (m, 2H), 1.36 (dd, J = 13.0, 3.2 |
| | | Hz, 2H), 1.23-1.20 (m, 1H); ESI MS(m/z): 384.05[M + 1]. |
| 227 | N-(methyl(oxo)(propyl)- | ¹H-NMR (400 MHz, DMSO-d₆) δ 9.55 (d, J = 8.6 Hz, 2H), |
| | 26-sulfaneylidene)-2- | 9.46-9.44 (m, 1H), 9.28 (s, 1H), 8.54 (t, J = 1.2 Hz, 1H), 7.94 |
| | (pyrimidin-5-yl)-2H- | (dd, J = 9.2, 1.6 Hz, 1H), 7.76-7.74 (m, 1H), 3.65-3.52 (m, |
| | indazole-5-carboxamide | 2H), 3.45 (s, 3H), 1.92-1.80 (m, 2H), 1.05 (t, J = 7.5 Hz, 3H) |
| | | ESI MS(m/z): 344.0[M + 1]. |
| 228 | N-(dimethyl(oxo)-λ⁶- | ¹H-NMR (400 MHz, DMSO-d₆) δ 9.54 (s, 2H), 9.45 (d, J = |
| | sulfaneylidene)-2- | 0.9 Hz, 1H), 9.28 (s, 1H), 8.54 (d, J = 0.9 Hz, 1H), 7.94 (dd, |
| | (pyrimidin-5-yl)-2H- | J = 9.2, 1.5 Hz, 1H), 7.76 (d, J = 9.2 Hz, 1H), 3.48 (s, 6H) |
| | indazole-5-carboxamide | ESI MS(m/z): 316.05[M + 1]. |
| 230 | N-(diisobutyl(oxo)-λ⁶- | ¹H-NMR (400 MHz, DMSO-d₆) δ 9.54 (s, 2H), 9.46 (s, 1H), |
| | sulfaneylidene)-2- | 9.28 (s, 1H), 8.54 (s, 1H), 7.94 (dd, J = 9.2, 1.6 Hz, 1H), 7.76 |
| | (pyrimidin-5-yl)-2H- | (d, J = 9.3 Hz, 1H), 3.62 (dd, J = 14.1, 6.5 Hz, 2H), 3.47 (dd, |
| | indazole-5-carboxamide | J = 14.2, 6.4 Hz, 2H), 2.36 (q, J = 6.7 Hz, 2H), 1.08 (q, J = |
| | | 3.3 Hz, 12H); ESI MS(m/z): 400.1[M + 1]. |
| 231 | N-(diethyl(oxo)-λ⁶- | ¹H-NMR (400 MHz, DMSO-d₆) δ 9.54 (s, 2H), 9.44 (d, J = |
| | sulfaneylidene)-2- | 1.0 Hz, 1H), 9.28 (s, 1H), 8.56 (t, J = 1.2 Hz, 1H), 7.95 (dd, |
| | (pyrimidin-5-yl)-2H- | J = 9.2, 1.6 Hz, 1H), 7.77-7.74 (m, 1H), 3.67-3.56 (m, 4H), |
| | indazole-5-carboxamide | 1.34 (t, J = 7.3 Hz, 6H); ESI MS(m/z): 344.0[M + 1]. |

### Example 3: Synthesis of N-(ethyl(isopropyl)(oxo)-λ⁶-sulfaneylidene)-2-(pyridin-3-yl)-2H-indazole-5-carboxamide 105

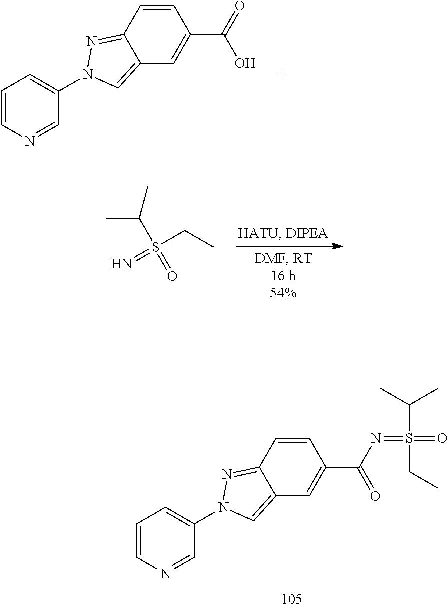

A solution of 2-(pyridin-3-yl)-2H-indazole-5-carboxylic acid (0.1 g, 0.418 mmol), ethyl(imino)(isopropyl)-λ⁶-sulfanone (0.057 g, 0.418 mmol) in dimethyl formamide (DMF) (2 mL) was treated with 1-[bis(dimethylamino)methylene]-1H-1,2,3-triazolo[4,5-b]pyridinium 3-oxide hexafluorophosphate (HATU) (0.159 g, 0.418 mmol) and N-ethyl-N-(propan-2-yl)propan-2-ammine (DIPEA) (0.110 mL, 0.627 mmol), and stirred at 25° C. for 16 h. After completion of the reaction, the reaction mixture was quenched by the addition of water (5 mL) and extracted with ethyl acetate (2×25 mL). The combined organic layers were dried over anhydrous sodium sulfate and concentrated under reduced pressure to obtain an oily residue. This residue was purified by CombiFlash® chromatography (normal phase; isocratic run with 100% ethyl acetate) to obtain the desired N-(ethyl(isopropyl)(oxo)-λ⁶-sulfaneylidene)-2-(pyridin-3-yl)-2H-indazole-5-carboxamide 105 (80 mg, 0.224 mmol, 53.7% yield).

TABLE 3
Representative compounds of the present disclosure were prepared according
to the suitable starting material and method as described in the Example 3.

| Compno. | Compound name | Analytical Data |
|---|---|---|
| 105 | N- | ¹H-NMR (400 MHz, DMSO-d₆) δ 9.38 (d, J = 1.0 Hz, 1H), |
| | (ethyl(isopropyl)(oxo)- | 9.33 (d, J = 2.2 Hz, 1H), 8.67 (dd, J = 4.6, 1.2 Hz, 1H), 8.55 (q, |
| | λ⁶-sulfaneylidene)-2- | J = 0.8 Hz, 1H), 8.49 (dq, J = 8.3, 1.4 Hz, 1H), 7.93 (dd, J = |
| | (pyridin-3-yl)-2H- | 9.2, 1.6 Hz, 1H), 7.74-7.72 (m, 1H), 7.66 (ddd, J = 8.3, 4.8, 0.6 |
| | indazole-5-carboxamide | Hz, 1H), 3.89-3.82 (m, 1H), 3.74-3.59 (m, 2H), 1.45-1.38 (m, |
| | | 6H), 1.37-1.27 (m, 4H); ESI MS (m/z): 357 [M + 1]. |
| 106 | N- | ¹H-NMR (400 MHz, DMSO-d₆) δ 9.38 (d, J = 1.0 Hz, 1H), |
| | (isopropyl(methyl)(oxo)- | 9.33 (d, J = 2.2 Hz, 1H), 8.67 (dd, J = 4.6, 1.5 Hz, 1H), 8.54 (q, |
| | λ⁶-sulfaneylidene)-2- | J = 0.8 Hz, 1H), 8.48 (dq, J = 8.3, 1.4 Hz, 1H), 7.93 (dd, J = |
| | (pyridin-3-yl)-2H- | 9.2, 1.6 Hz, 1H), 7.74 (dt, J = 9.2, 0.9 Hz, 1H), 7.66 (ddd, J = |
| | indazole-5-carboxamide | 8.4, 4.8, 0.7 Hz, 1H), 3.79-3.72 (m, 1H), 3.44 (s, 3H), 1.43 (q, |
| | | J = 3.3 Hz, 6H); ESI MS (m/z): 343[M + 1]. |
| 107 | N-(diethyl(oxo)-λ⁶- | ¹H-NMR (400 MHz, DMSO-d₆) δ 9.38 (d, J = 0.7 Hz, 1H), |
| | sulfaneylidene)-2- | 9.33 (d, J = 2.2 Hz, 1H), 8.67 (dd, J = 4.8, 1.3 Hz, 1H), 8.55 (q, |
| | (pyridin-3-yl)-2H- | J = 0.8 Hz, 1H), 8.49 (dq, J = 8.3, 1.4 Hz, 1H), 7.93 (dd, J = |
| | indazole-5-carboxamide | 9.2, 1.6 Hz, 1H), 7.73 (dt, J = 9.1, 0.9 Hz, 1H), 7.66 (ddd, J = |
| | | 8.3, 4.6, 0.7 Hz, 1H), 3.67-3.55 (m, 4H), 1.34 (t, J = 7.3 Hz, |
| | | 7H); ESI MS (m/z): 343[M + 1]. |

### Example 4: Synthesis of N,N 4-trimethyl-N′-(2-(pyridin-3-yl)-2H-indazol-5-yl) benzenesulfonimidamide 93

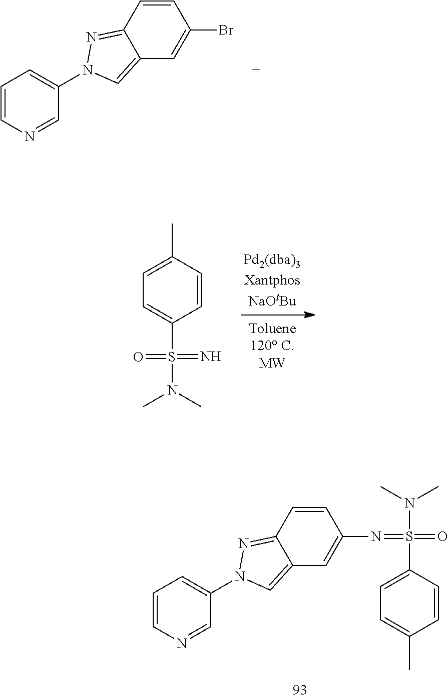 [SMILES: Brc1ccc2nn(-c3cccnc3)cc2c1 ; Cc1ccc(S(=N)(=O)N(C)C)cc1 ; Cc1ccc(S(=O)(=Nc2ccc3nn(-c4cccnc4)cc3c2)N(C)C)cc1]

A solution of 5-bromo-2-(pyridin-3-yl)-2H-indazole (0.27 g, 0.985 mmol), N,N 4-trimethylbenzenesulfonimidamide (0.195 g, 0.985 mmol) in toluene (5 mL) was treated with tris(dibenzylideneacetone)dipalladium(0) (Pd₂(dba)₃) (0.045 g, 0.049 mmol), (9,9-dimethyl-9H-xanthene-4,5-diyl)bis(diphenylphosphane) (Xantphos) (0.057 g, 0.098 mmol) and sodium tert-butoxide (0.095 g, 0.985 mmol), and irradiated in a microwave (MW) at 120° C. for 1 h. After completion of the reaction, the reaction mixture was quenched by the addition of water (10 mL) and extracted with ethyl acetate (2×25 mL); the combined organic layers were dried over anhydrous sodium sulfate and concentrated under reduced pressure to obtain an oily residue. This residue was purified by Combiflash® chromatography to obtain N,N 4-trimethyl-N′-(2-(pyridin-3-yl)-2H-indazol-5-yl)benzenesulfonimidamide 93 (133 mg, 0.340 mmol, 34.5 00 yield).

TABLE 4
Representative compounds of the present disclosure were prepared according
to the suitable starting material and method as described in the Example 4.

| Comp | | |
|---|---|---|
| no. | Compound name | Analytical Data |
| 93 | N,N,4-trimethyl-N′-(2-(pyridin- | ¹H-NMR (400 MHz, DMSO-d₆) δ 9.27 (d, J = 2.4 Hz, |
| | 3-yl)-2H-indazol-5- | 1H), 8.98 (d, J = 0.7 Hz, 1H), 8.61 (dd, J = 4.6, 1.5 |
| | yl)benzenesulfonimidamide | Hz, 1H), 8.42 (dq, J = 8.3, 1.4 Hz, 1H), 7.85 (d, J = |
| | | 8.3 Hz, 2H), 7.64-7.60 (m, 2H), 7.47 (d, J = 8.1 Hz, |
| | | 2H), 7.34 (d, J = 1.2 Hz, 1H), 7.21 (dd, J = 9.3, 2.0 |
| | | Hz, 1H), 2.61 (s, 6H), 2.42 (s, 3H) |
| | | ESI MS (m/z): 392 [M + 1]. |
| 94 | N′-(2-(5-fluoropyridin-3-yl)-2H- | ¹H-NMR (400 MHz, DMSO-d₆) δ 9.20 (s, 1H), 9.03 |
| | indazol-5-yl)-N,N,4- | (d, J = 0.7 Hz, 1H), 8.64 (d, J = 2.4 Hz, 1H), 8.43 (dt, |
| | trimethylbenzenesulfonimidamide | J = 10.1, 2.4 Hz, 1H), 7.85 (d, J = 8.3 Hz, 2H), 7.63 |
| | | (d, J = 9.3 Hz, 1H), 7.47 (d, J = 8.1 Hz, 2H), 7.33 (t, |
| | | J = 1.0 Hz, 1H), 7.22 (dd, J = 9.3, 2.0 Hz, 1H), 2.61 (s, |
| | | 6H), 2.42 (s, 3H); ESI MS (m/z): 410 [M + 1]. |
| 95 | N,N,4-trimethyl-N′-(2-(pyrimidin- | ¹H-NMR (400 MHz, DMSO-d₆) δ 9.48 (s, 2H), 9.22 |
| | 5-yl)-2H-indazol-5- | (s, 1H), 9.04 (d, J = 0.7 Hz, 1H), 7.85 (d, J = 8.3 Hz, |
| | yl)benzenesulfonimidamide | 2H), 7.65 (d, J = 9.3 Hz, 1H), 7.47 (d, J = 8.1 Hz, |
| | | 2H), 7.34 (d, J = 1.2 Hz, 1H), 7.23 (dd, J = 9.2, 2.1 |
| | | Hz, 1H), 2.61 (s, 6H), 2.42 (s, 3H) |
| | | ESI MS (m/z): 393[M + 1]. |
| 96 | N,N,4-trimethyl-N′-(2-(pyridin-3- | ¹H-NMR (400 MHz, DMSO-d₆) δ 9.41 (d, J = 2.4 Hz, |
| | yl)-2H-indazol-4- | 1H), 9.31 (s, 1H), 8.63 (dd, J = 4.8, 1.3 Hz, 1H), 8.55 |
| | yl)benzenesulfonimidamide | (dq, J = 8.4, 1.3 Hz, 1H), 7.97 (d, J = 8.3 Hz, 2H), |
| | | 7.63 (dd, J = 8.4, 4.8 Hz, 1H), 7.49 (d, J = 8.1 Hz, |
| | | 2H), 7.25 (d, J = 8.8 Hz, 1H), 7.16 (dd, J = 8.6, 7.1 |
| | | Hz, 1H), 6.76 (d, J = 7.1 Hz, 1H), 2.63 (s, 6H), 2.44 |
| | | (d, J = 7.6 Hz, 3H); ESI MS (m/z): 392[M + 1]. |
| 97 | N,N,4-trimethyl-N′-(2-(pyrimidin- | ¹H-NMR (400 MHz, DMSO-d₆) δ 9.60 (s, 2H), 9.39 |
| | 5-yl)-2H-indazol-4- | (d, J = 0.7 Hz, 1H), 9.24 (s, 1H), 7.97 (d, J = 8.3 Hz, |
| | yl)benzenesulfonimidamide | 2H), 7.49 (d, J = 8.1 Hz, 2H), 7.26 (d, J = 8.6 Hz, |
| | | 1H), 7.18 (dd, J = 8.6, 7.1 Hz, 1H), 6.76 (d, J = 6.8 |
| | | Hz, 1H), 2.65 (d, J = 10.0 Hz, 6H), 2.45 (d, J = 7.8 |
| | | Hz, 3H); ESI MS (m/z): 393[M + 1]. |
| 98 | N′-(2-(5-fluoropyridin-3-yl)-2H- | ¹H-NMR (400 MHz, DMSO-d₆) δ 9.36 (s, 1H), 9.33 |
| | indazol-4-yl)-N,N,4- | (s, 1H), 8.66 (d, J = 2.4 Hz, 1H), 8.58 (dt, J = 10.2, |
| | trimethylbenzenesulfonimidamide | 2.3 Hz, 1H), 7.97 (d, J = 8.3 Hz, 2H), 7.50 (d, J = 8.1 |
| | | Hz, 2H), 7.24 (d, J = 8.6 Hz, 1H), 7.17 (dd, J = 8.7, |
| | | 7.0 Hz, 1H), 6.76 (d, J = 6.8 Hz, 1H), 2.63 (s, 6H), |
| | | 2.44 (s, 3H); ESI MS (m/z): 410[M + 1]. |
| 134 | N-ethyl-N-methyl-N′-(2- | ¹H-NMR (400 MHz, DMSO-d₆) δ 9.55 (s, 2H), 9.22 |
| | (pyrimidin-5-yl)-2H-indazol-4- | (s, 1H), 9.15 (d, J = 0.7 Hz, 1H), 7.21-7.13 (m, 2H), |
| | yl)ethanesulfonimidamide | 6.65 (dd, J = 6.8, 1.0 Hz, 1H), 3.33 (s, 2H), 3.27 (dd, |
| | | J = 7.2, 5.7 Hz, 2H), 2.81 (s, 3H), 1.39 (t, J = 7.3 Hz, |
| | | 3H), 1.01 (t, J = 7.2 Hz, 3H); ESI MS (m/z): 345.1 |
| | | [M + 1]. |
| 135 | N-ethyl-N-methyl-N′-(2- | ¹H-NMR (400 MHz, DMSO-d₆) δ 9.47 (s, 2H), 9.21 |
| | (pyrimidin-5-yl)-2H-indazol-5- | (s, 1H), 9.01 (d, J = 0.9 Hz, 1H), 7.59-7.57 (m, 1H), |
| | yl)cyclopropanesulfonimidamide | 7.18 (q, J = 0.9 Hz, 1H), 7.06 (dd, J = 9.2, 1.8 Hz, |
| | | 1H), 3.28-3.20 (m, 3H), 2.81-2.77 (m, 4H), 1.11-0.97 |
| | | (m, 6H); ESI MS (m/z): 356.55 [M + 1]. |

### Example 5: Synthesis of 5-methoxy-4-methyl-2-(2-(pyridin-3-yl)-2H-indazol-5-yl)-2,4-dihydro-3H-1,2,4-triazol-3-one 110

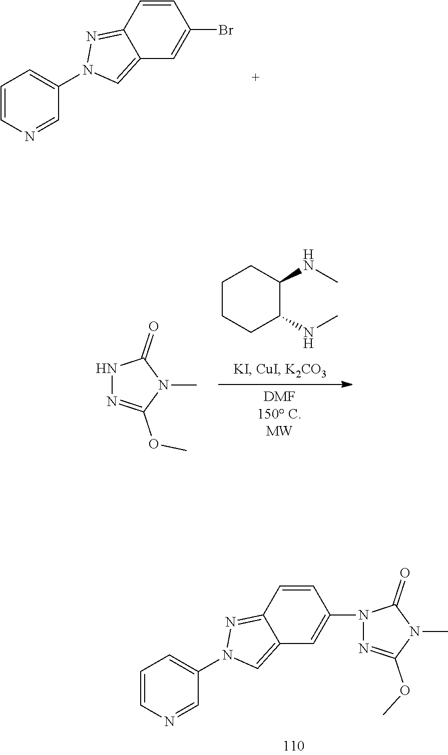

To a solution of 5-bromo-2-(pyridin-3-yl)-2H-indazole (0.6 g, 2.189 mmol), 5-methoxy-4-methyl-2,4-dihydro-3H-1,2,4-triazol-3-one (0.791 g, 6.13 mmol) in dimethyl formamide (DMF) (6 mL), copper(I) iodide (CuI) (0.083 g, 0.438 mmol), trans-1,2-bis(methylamino)cyclohexane (0.249 g, 0.876 mmol) potassium iodide (KI) (0.109 g, 0.657 mmol), and potassium carbonate (K₂CO₃) (0.908 g, 6.57 mmol) were added and the mixture was irradiated in a microwave device (MW) at 150° C. for 1 h. After completion of the reaction, the reaction mixture was quenched by the addition of water (20 mL) and extracted with ethyl acetate (3×25 mL). The combined organic layers were dried over anhydrous sodium sulfate and concentrated under reduced pressure to obtain a gummy residue. This residue was purified by CombiFlash® chromatography to obtain 5-methoxy-4-methyl-2-(2-(pyridin-3-yl)-2H-indazol-5-yl)-2,4-dihydro-3H-1,2,4-triazol-3-one 110 (0.184 g, 0.571 mmol, 26.1% yield).

TABLE 5
Representative compounds of the present disclosure were prepared according
to the suitable starting material and method as described in the Example 5.

| Comp | | |
|---|---|---|
| no. | Compound name | Analytical Data |
| 110 | 5-methoxy-4-methyl-2- | ¹H-NMR (400 MHz, DMSO-d₆) δ 9.33 (d, J = 2.4 Hz, 1H), 9.27 |
| | (2-(pyridin-3-yl)-2H- | (d, J = 0.9 Hz, 1H), 8.67 (dd, J = 4.7, 1.4 Hz, 1H), 8.49 (dq, J = |
| | indazol-5-yl)-2,4- | 8.3, 1.4 Hz, 1H), 7.87-7.84 (m, 1H), 7.77 (q, J = 0.9 Hz, 1H), |
| | dihydro-3H-1,2,4-triazol- | 7.66 (ddd, J = 8.3, 4.7, 0.7 Hz, 1H), 7.38 (dd, J = 9.2, 2.1 Hz, |
| | 3-one | 1H), 3.04 (s, 3H), 3.01 (s, 3H); ESI MS (m/z): 322.6[M + 1]. |
| 111 | 5-methoxy-4-methyl-2- | ¹H-NMR (400 MHz, DMSO-d₆) δ 9.25 (s, 1H), 9.09-9.20 |
| | (2-(5-methylpyridin-3- | (m,1H), 8.58 (s, 1H), 8.35 (s, 1H), 7.84 (d, J = 9.3 Hz, 1H), 7.76 |
| | yl)-2H-indazol-5-yl)-2,4- | (d, J = 1.2 Hz, 1H), 7.37 (dd, J = 9.3, 2.0 Hz, 1H), 3.02 (d, J = |
| | dihydro-3H-1,2,4-triazol- | 10.5 Hz, 6H), 2.44 (s, 3H); ESI MS (m/z): 336.7 [M + 1]. |
| | 3-one | |
| 112 | 2-(2-(5-fluoropyridin-3- | ¹H-NMR (400 MHz, DMSO-d₆) δ 9.31 (d, J = 0.7 Hz, 1H), 9.26 |
| | yl)-2H-indazol-5-yl)-5- | (s, 1H), 8.70 (d, J = 2.4 Hz, 1H), 8.52 (dt, J = 9.9, 2.3 Hz, 1H), |
| | methoxy-4-methyl-2,4- | 7.85 (d, J = 9.3 Hz, 1H), 7.76 (t, J = 1.0 Hz, 1H), 7.40 (dd, J = |
| | dihydro-3H-1,2,4-triazol- | 9.3, 2.0 Hz, 1H), 3.04 (s, 3H), 3.01 (s, 3H); ESI MS (m/z): 341 |
| | 3-one | [M + 1]. |
| 113 | 5-methoxy-4-methyl-2- | ¹H-NMR (400 MHz, DMSO-d₆) δ 8.84 (d, J = 0.9 Hz, 1H), 8.65 |
| | (2-(2-methylpyridin-3- | (dd, J = 4.6, 1.5 Hz, 1H), 7.97 (dd, J = 7.9, 1.5 Hz, 1H), 7.85- |
| | yl)-2H-indazol-5-yl)-2,4- | 7.82 (m, 1H), 7.80 (q, J = 0.9 Hz, 1H), 7.51-7.48 (m, 1H), 7.36 |
| | dihydro-3H-1,2,4-triazol- | (dd, J = 9.3, 2.0 Hz, 1H), 3.04 (d, J = 5.2 Hz, 3H), 3.01 (s, 3H), |
| | 3-one | 2.42 (s, 3H); ESI MS (m/z): 336.9 [M + 1]. |
| 114 | 5-methoxy-4-methyl-2- | ¹H-NMR (400 MHz, DMSO-d₆) δ 8.85 (d, J = 0.7 Hz, 1H), 8.69 |
| | (2-(4-methylpyridin-3- | (s, 1H), 8.61 (d, J = 4.9 Hz, 1H), 7.87-7.83 (m, 1H), 7.80 (q, J = |
| | yl)-2H-indazol-5-yl)-2,4- | 0.9 Hz, 1H), 7.55 (d, J = 5.1 Hz, 1H), 7.36 (dd, J = 9.2, 2.1 Hz, |
| | dihydro-3H-1,2,4-triazol- | 1H), 3.04 (s, 3H), 3.01 (s, 3H), 2.29 (s, 3H) |
| | 3-one | ESI MS (m/z): 336.9 [M + 1]. |
| 115 | 5-methoxy-4-methyl-2- | ¹H-NMR (400 MHz, DMSO-d₆) δ 9.21 (d, J = 0.9 Hz, 1H), 9.17 |
| | (2-(6-methylpyridin-3- | (d, J = 2.8 Hz, 1H), 8.36 (dd, J = 8.6, 2.8 Hz, 1H), 7.85-7.83 (m, |
| | yl)-2H-indazol-5-yl)-2,4- | 1H), 7.75 (q, J = 0.9 Hz, 1H), 7.50 (d, J = 8.6 Hz, 1H), 7.36 (dd, |
| | dihydro-3H-1,2,4-triazol- | J = 9.3, 2.0 Hz, 1H), 3.03 (s, 3H), 3.01 (s, 3H), 2.56 (s, 3H) |
| | 3-one | ESI MS (m/z): 336.9 [M + 1]. |
| 116 | 2-(2-(2,6- | ¹H-NMR (400 MHz, DMSO-d₆) δ 8.78 (d, J = 0.9 Hz, 1H), 7.84 |
| | dimethylpyridin-3-yl)- | (t, J = 1.1 Hz, 1H), 7.82-7.81 (m, 1H), 7.78 (q, J = 0.9 Hz, 1H), |
| | 2H-indazol-5-yl)-5- | 7.36-7.31 (m, 2H), 3.04 (d, J = 5.8 Hz, 3H), 3.01 (s, 3H), 2.55 |
| | methoxy-4-methyl-2,4- | (s, 3H), 2.36 (s, 3H); ESI MS (m/z): 350.9 [M + 1]. |
| | dihydro-3H-1,2,4-triazol- | |
| | 3-one | |
| 117 | 5-methoxy-2-(2-(5- | ¹H-NMR (400 MHz, DMSO-d₆) δ 9.30 (d, J = 0.9 Hz, 1H), 8.94 |
| | methoxypyridin-3-yl)- | (s, 1H), 8.40 (s, 1H), 8.07 (t, J = 2.4 Hz, 1H), 7.87-7.84 (m, 1H), |
| | 2H-indazol-5-yl)-4- | 7.76 (q, J = 0.9 Hz, 1H), 7.38 (dd, J = 9.3, 2.0 Hz, 1H), 3.96 (s, |
| | methyl-2,4-dihydro-3H- | 3H), 3.04 (s, 3H), 3.01 (s, 3H) |
| | 1,2,4-triazol-3-one | ESI MS (m/z): 353 [M + 1]. |
| 118 | 5-methoxy-4-methyl-2- | ¹H-NMR (400 MHz, DMSO-d₆) δ 9.66 (d, J = 2.4 Hz, 1H), 9.44 |
| | (2-(5- | (d, J = 0.7 Hz, 1H), 9.07 (d, J = 0.7 Hz, 1H), 8.89 (t, J = 1.8 Hz, |
| | (trifluoromethyl)pyridin- | 1H), 7.88-7.86 (m, 1H), 7.78 (q, J = 0.9 Hz, 1H), 7.41 (dd, J = |
| | 3-yl)-2H-indazol-5-yl)- | 9.2, 2.1 Hz, 1H), 3.03 (d, J = 5.9 Hz, 3H), 3.01 (s, 3H) |
| | 2,4-dihydro-3H-1,2,4- | ESI MS (m/z): 391 [M + 1]. |
| | triazol-3-one | |
| 119 | 5-methoxy-4-methyl-2- | ¹H-NMR (400 MHz, DMSO-d₆) δ 9.54 (s, 2H), 9.33 (d, J = 1.0 |
| | (2-(pyrimidin-5-yl)-2H- | Hz, 1H), 9.28 (s, 1H), 7.87 (d, J = 9.3 Hz, 1H), 7.79 (d, J = 1.5 |
| | indazol-5-yl)-2,4- | Hz, 1H), 7.41 (dd, J = 9.3, 2.0 Hz, 1H), 3.04 (s, 3H), 3.01 (s, |
| | dihydro-3H-1,2,4-triazol- | 3H); ESI MS (m/z): 324 [M + 1]. |
| | 3-one | |
| 120 | 2-(2-(5-chloropyridin-3- | ¹H-NMR (400 MHz, DMSO-d₆) δ 9.99 (q, J = 1.2 Hz, 1H), 9.36 |
| | yl)-2H-indazol-5-yl)-5- | (dd, J = 5.9, 1.0 Hz, 1H), 9.11 (d, J = 1.0 Hz, 1H), 8.27 (q, J = |
| | methoxy-4-methyl-2,4- | 2.9 Hz, 1H), 7.60-7.57 (m, 1H), 7.13 (q, J = 1.0 Hz, 1H), 7.01 |
| | dihydro-3H-1,2,4-triazol- | (dd, J = 9.3, 2.0 Hz, 1H), 3.37 (dd, J = 10.1, 5.7 Hz, 2H), 3.16 |
| | 3-one | (s, 3H), 1.79-1.72 (m, 2H), 1.38-1.26 (m, 4H), 0.84 (t, J = 7.1 |
| | | Hz, 3H); ESI MS (m/z): 356.6 [M + 1]. |
| 121 | 4-benzyl-5-methoxy-2- | ¹H-NMR (400 MHz, CHLOROFORM-D) δ 9.20 (s, 1H), 8.71 |
| | (2-(pyridin-3-yl)-2H- | (s, 1H), 8.50 (s, 1H), 8.29 (d, J = 8.1 Hz, 1H), 7.86 (d, J = 9.3 |
| | indazol-5-yl)-2,4- | Hz, 1H), 7.68 (d, J = 1.2 Hz, 1H), 7.53-7.48 (m, 3H), 7.38-7.28 |
| | dihydro-3H-1,2,4-triazol- | (m, 4H), 4.78 (s, 2H), 3.16 (s, 3H); ESI MS (m/z): 399.00 |
| | 3-one | [M + 1]. |
| 122 | 4-(2-fluorobenzyl)-5- | ¹H-NMR (400 MHz, DMSO-d₆) δ 9.33 (d, J = 2.4 Hz, 1H), 9.28 |
| | methoxy-2-(2-(pyridin-3- | (d, J = 0.7 Hz, 1H), 8.67 (dd, J = 4.8, 1.3 Hz, 1H), 8.49 (dq, J = |
| | yl)-2H-indazol-5-yl)-2,4- | 8.3, 1.4 Hz, 1H), 7.87 (d, J = 9.3 Hz, 1H), 7.82 (d, J = 1.2 Hz. |
| | dihydro-3H-1,2,4-triazol- | 1H), 7.66 (dd, J = 8.1, 4.4 Hz, 1H), 7.41-7.35 (m, 3H), 7.24- |
| | 3-one | 7.19 (m, 2H), 4.75 (s, 2H), 3.07 (s, 3H); ESI MS (m/z): 417.15 |
| | | [M + 1]. |
| 123 | 4-ethyl-5-methoxy-2-(2- | H-NMR (400 MHz, DMSO-d₆) δ 9.35 (d, J = 2.4 Hz, 1H), 9.29 |
| | (pyridin-3-yl)-2H- | (d, J = 1.0 Hz, 1H), 8.69 (dd, J = 4.8, 1.3 Hz, 1H), 8.51 (dq, J = |
| | indazol-5-yl)-2,4- | 8.3, 1.4 Hz, 1H), 7.89-7.86 (m, 1H), 7.80 (q, J = 0.9 Hz, 1H), |
| | dihydro-3H-1,2,4-triazol- | 7.69-7.66 (m, 1H), 7.39 (dd, J = 9.3, 2.0 Hz, 1H), 3.56 (q, J = |
| | 3-one | 7.2 Hz, 2H), 3.06 (s, 3H), 1.21 (t, J = 7.2 Hz, 3H); ESI MS (m/z): |
| | | 337.00 [M + 1]. |
| 124 | 4-(4-fluorobenzyl)-5- | ′H-NMR (400 MHz, DMSO-d₆) δ 9.33 (d, J = 2.4 Hz, 1H), 9.28 |
| | methoxy-2-(2-(pyridin-3- | (d, J = 1.0 Hz, 1H), 8.67 (d, J = 4.6 Hz, 1H), 8.49 (dq, J = 8.3, |
| | yl)-2H-indazol-5-yl)-2,4- | 1.4 Hz, 1H), 7.87-7.85 (m, 1H), 7.81 (q, J = 1.0 Hz, 1H), 7.66 |
| | dihydro-3H-1,2,4-triazol- | (q, J = 4.3 Hz, 1H), 7.42-7.36 (m, 3H), 7.22-7.17 (m, 2H), 4.68 |
| | 3-one | (s, 2H), 3.06 (s, 3H); ESI MS (m/z): 417.15 [M + 1]. |

### Example 6: Synthesis of 4-ethyl-2-(2-(5-fluoropyridin-3-yl)-2H-indazol-4-yl)-5-methoxy-2,4-dihydro-3H-1,2,4-triazol-3-one 130

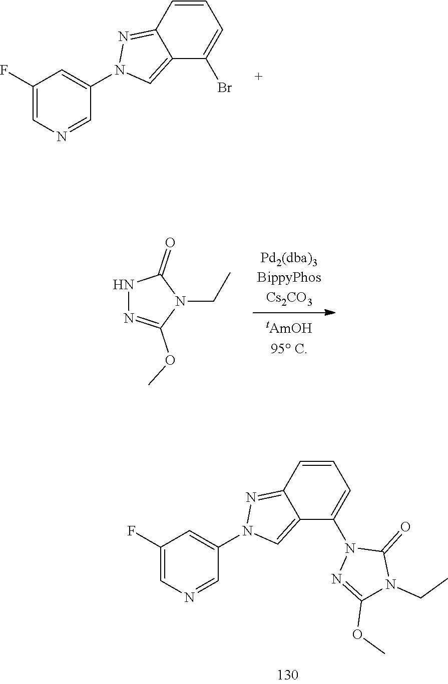

4-Bromo-2-(5-fluoropyridin-3-yl)-2H-indazole (300 mg, 1.027 mmol), 4-ethyl-5-methoxy-2,4-dihydro-3H-1,2,4-triazol-3-one (162 mg, 1.130 mmol), tris(dibenzylideneacetone)dipalladium (Pd₂(dba)₃) (103 mg, 0.113 mmol), 5-(di-tert-butylphosphino)-1′,3′,5′-triphenyl-1′H-[1,4′]bipyrazole (BippyPhos) (97 mg, 0.191 mmol), and cesium carbonate (Cs₂CO₃) (970 mg, 2.98 mmol) were charged into a flask, equipped with a condenser, under N₂ atmosphere and this reaction mixture was stirred. Tertiary-amyl alcohol (20 mL) was added and the resulting solution was heated at 95° C. under N₂ for 16 h. After completion of the reaction, the reaction mixture was concentrated under reduced pressure to obtain a dark residue, which was purified by CombiFlash® chromatography to obtain 4-ethyl-2-(2-(5-fluoropyridin-3-yl)-2H-indazol-4-yl)-5-methoxy-2,4-dihydro-3H-1,2,4-triazol-3-one 130 (132.5 mg, 0.374 mmol, 36.4% yield).

TABLE 6
Representative compounds of the present disclosure were prepared according to
the suitable starting material and methods as described in the Example 6.

| Comp | | |
|---|---|---|
| no. | Compound name | Analytical Data |
| 125 | 5-methoxy-4-methyl-2- | ¹H-NMR (400 MHz, DMSO-d₆) δ 9.33 (d, J = 2.2 Hz, 1H), |
| | (2-(pyridin-3-yl)-2H- | 9.29 (d, J = 0.7 Hz, 1H), 8.67 (dd, J = 4.6, 1.5 Hz, 1H), 8.50 |
| | indazol-4-yl)-2,4- | (dq, J = 8.3, 1.4 Hz, 1H), 7.71 (dd, J = 7.3, 0.5 Hz, 1H), 7.65 |
| | dihydro-3H-1,2,4-triazol- | (ddd, J = 8.3, 4.8, 0.6 Hz, 1H), 7.56-7.54 (m, 1H), 7.37 (dd, |
| | 3-one | J = 8.6, 7.3 Hz, 1H), 4.18 (s, 3H), 3.15 (s, 3H); ESI MS (m/z): |
| | | 322.40 [M + 1]. |
| 126 | 4-(3-fluorobenzyl)-5- | ¹H-NMR (400 MHz, CHLOROFORM-D) δ 9.18 (s, 1H), 8.68 |
| | methoxy-2-(2-(pyridin-3- | (s, 1H), 8.49 (s, 1H), 8.26 (d, J = 8.1 Hz, 1H), 7.84 (d, J = 9.3 |
| | yl)-2H-indazol-5-yl)-2,4- | Hz, 1H), 7.67 (d, J = 1.2 Hz, 1H), 7.50 (d, J = 2.7 Hz, 1H), |
| | dihydro-3H-1,2,4-triazol- | 7.34-7.27 (m, 2H), 7.24 (d, J = 7.6 Hz, 1H), 7.18 (dd, J = 9.4, |
| | 3-one | 2.1 Hz, 1H), 7.02-6.97 (m, 1H), 4.75 (s, 2H), 3.16-3.14 (m, |
| | | 3H); ESI MS (m/z): 417.00 [M + 1]. |
| 127 | 4-cyclopropyl-5- | ¹H-NMR (400 MHz, DMSO-d₆) δ 9.31 (d, J = 2.4 Hz, 1H), |
| | methoxy-2-(2-(pyridin-3- | 9.20 (s, 1H), 8.65 (dd, J = 4.8, 1.3 Hz, 1H), 8.46 (dq, J = 8.4, |
| | yl)-2H-indazol-5-yl)-2,4- | 1.3 Hz, 1H), 8.12 (d, J = 1.2 Hz, 1H), 7.94 (dd, J = 9.3, 2.0 |
| | dihydro-3H-1,2,4-triazol- | Hz, 1H), 7.81 (d, J = 9.3 Hz, 1H), 7.64 (dd, J = 8.4, 4.8 Hz, |
| | 3-one | 1H), 4.05 (d, J = 5.9 Hz, 3H), 2.82-2.77 (m, 1H), 0.97-0.89 |
| | | (m, 4H); ESI MS (m/z): 349.900[M + 1]. |
| 128 | 4-cyclopropyl-5- | ¹H-NMR (400 MHz, DMSO-d₆) δ 9.55 (s, 2H), 9.36 (d, J = |
| | methoxy-2-(2- | 1.0 Hz, 1H), 9.28 (s, 1H), 7.72 (d, J = 6.8 Hz, 1H), 7.55 (d, J = |
| | (pyrimidin-5-yl)-2H- | 8.8 Hz, 1H), 7.39 (dd, J = 8.7, 7.5 Hz, 1H), 4.17 (s, 3H), 2.84 |
| | indazol-4-yl)-2,4- | (s, 1H), 0.95 (dd, J = 9.4, 2.8 Hz, 4H) |
| | dihydro-3H-1,2,4-triazol- | ESI MS (m/z): 350.20 [M + 1]. |
| | 3-one | |
| 129 | 4-ethyl-5-methoxy-2-(2- | ¹H-NMR (400 MHz, DMSO-d₆) δ 9.56 (s, 2H), 9.38 (d, J = |
| | (pyrimidin-5-yl)-2H- | 1.0 Hz, 1H), 9.29 (s, 1H), 7.74 (d, J = 7.3 Hz, 1H), 7.56 (d, J = |
| | indazol-4-yl)-2,4- | 8.8 Hz, 1H), 7.40 (dd, J = 8.7, 7.5 Hz, 1H), 4.20 (s, 3H), 3.65 |
| | dihydro-3H-1,2,4-triazol- | (q, J = 7.3 Hz, 2H), 1.24 (t, J = 7.2 Hz, 3H) |
| | 3-one | ESI MS (m/z): 338.05 [M + 1]. |
| 130 | 4-ethyl-2-(2-(5- | ¹H-NMR (400 MHz, DMSO-d₆) δ 9.34 (d, J = 1.0 Hz, 1H), |
| | fluoropyridin-3-yl)-2H- | 9.27 (s, 1H), 8.71 (d, J = 2.2 Hz, 1H), 8.55 (dt, J = 9.9, 2.4 Hz, |
| | indazol-4-yl)-5-methoxy- | 1H), 7.71 (d, J = 6.6 Hz, 1H), 7.55 (d, J = 8.6 Hz, 1H), 7.39 |
| | 2,4-dihydro-3H-1,2,4- | (dd, J = 8.8, 7.3 Hz, 1H), 4.20 (s, 3H), 3.65 (q, J = 7.2 Hz, |
| | triazol-3-one | 2H), 1.24 (t, J = 7.2 Hz, 3H); ESI MS (m/z): 355.20 [M + 1]. |
| 131 | 4-ethyl-5-methoxy-2-(2- | ¹H-NMR (400 MHz, DMSO-d₆) δ 9.52 (s, 2H), 9.27 (d, J = |
| | (pyrimidin-5-yl)-2H- | 1.0 Hz, 1H), 9.26 (s, 1H), 8.15 (q, J = 0.9 Hz, 1H), 7.98 (dd, |
| | indazol-5-yl)-2,4- | J = 9.5, 2.0 Hz, 1H), 7.85 (d, J = 9.5 Hz, 1H), 4.07 (s, 3H), 3.61 |
| | dihydro-3H-1,2,4-triazol- | (q, J = 7.3 Hz, 2H), 1.22 (t, J = 7.2 Hz, 4H) |
| | 3-one | ESI MS (m/z): 338.20 [M + 1]. |
| 132 | 4-cyclopropyl-2-(2-(5- | ¹H-NMR (400 MHz, DMSO-d₆) δ 9.24 (d, J = 0.7 Hz, 2H), |
| | fluoropyridin-3-yl)-2H- | 8.68 (d, J = 2.2 Hz, 1H), 8.49-8.47 (m, 1H), 8.11 (t, J = 1.0 |
| | indazol-5-yl)-5-methoxy- | Hz, 1H), 7.95 (dd, J = 9.3, 2.0 Hz, 1H), 7.81 (d, J = 9.5 Hz, |
| | 2,4-dihydro-3H-1,2,4- | 1H), 4.04 (s, 3H), 2.80 (s, 1H), 0.95-0.91 (m, 4H) |
| | triazol-3-one | ESI MS (m/z): 367.20 [M + 1]. |
| 133 | 4-ethyl-2-(2-(5- | ¹H-NMR (400 MHz, DMSO-d₆) δ 9.25-9.24 (m, 2H), 8.68 (d, |
| | fluoropyridin-3-yl)-2H- | J = 2.2 Hz, 1H), 8.48 (dt, J = 9.9, 2.3 Hz, 1H), 8.13 (q, J = 0.9 |
| | indazol-5-yl)-5-methoxy- | Hz, 1H), 7.97 (dd, J = 9.4, 2.1 Hz, 1H), 7.82 (d, J = 9.5 Hz, |
| | 2,4-dihydro-3H-1,2,4- | 1H), 4.07 (s, 3H), 3.61 (q, J = 7.2 Hz, 2H), 1.21 (t, J = 7.2 Hz, |
| | triazol-3-one | 3H); ESI MS (m/z): 355.00 [M + 1]. |
| 319 | 2-(2-(5-fluoropyridin-3- | ¹H-NMR (400 MHz, DMSO-d₆) δ 9.25 (t, J = 1.3 Hz, 2H), |
| | yl)-2H-indazol-5-yl)-5- | 8.69-8.68 (m, 1H), 8.49 (dt, J = 10.0, 2.3 Hz, 1H), 8.14 (q, J = |
| | methoxy-4-(3- | 0.9 Hz, 1H), 7.97 (dd, J = 9.4, 2.1 Hz, 1H), 7.83-7.81 (m, 1H), |
| | methylbutan-2-yl)-2,4- | 4.06 (s, 3H), 3.72 (td, J = 7.0, 2.9 Hz, 1H), 2.17-2.12 (m, 1H), |
| | dihydro-3H-1,2,4-triazol- | 1.40 (d, J = 7.1 Hz, 3H), 0.97 (d, J = 6.6 Hz, 3H), 0.80 (d, J = |
| | 3-one | 6.8 Hz, 3H); ESI MS (m/z): 397.10 [M + 1]. |
| 320 | 4-cyclopropyl-2-(2-(5- | ¹H-NMR (400 MHz, DMSO-d₆) δ 9.31 (d, J = 1.0 Hz, 1H), |
| | fluoropyridin-3-yl)-2H- | 9.27 (s, 1H), 8.71 (d, J = 2.7 Hz, 1H), 8.54 (dd, J = 10.0, 2.2 |
| | indazol-4-yl)-5-methoxy- | Hz, 1H), 7.69 (d, J = 6.8 Hz, 1H), 7.54 (d, J = 8.6 Hz, 1H), |
| | 2,4-dihydro-3H-1,2,4- | 7.38 (dd, J = 8.8, 7.3 Hz, 1H), 4.17 (s, 3H), 2.84 (s, 1H), 0.96 |
| | triazol-3-one | (d, J = 6.6 Hz, 4H); ESI MS (m/z): 367.05 [M + 1]. |
| 321 | 4-cyclopropyl-5- | ¹H-NMR (400 MHz, DMSO-d₆) δ 9.33 (d, J = 2.7 Hz, 1H), |
| | methoxy-2-(2-(pyridin-3- | 9.27 (s, 1H), 8.67 (d, J = 3.4 Hz, 1H), 8.51-8.48 (m, 1H), 7.69 |
| | yl)-2H-indazol-4-yl)-2,4- | (d, J = 6.8 Hz, 1H), 7.65 (dd, J = 8.4, 4.8 Hz, 1H), 7.54 (d, J = |
| | dihydro-3H-1,2,4-triazol- | 8.8 Hz, 1H), 7.38-7.34 (m, 1H), 4.16 (s, 3H), 2.84 (s, 1H), |
| | 3-one | 0.97-0.94 (m, 4H); ESI MS (m/z): 349.05 [M + 1]. |
| 322 | 5-methoxy-4-(3- | ¹H-NMR (400 MHz, DMSO-d₆) δ 9.32 (d, J = 2.2 Hz, 1H), |
| | methylbutan-2-yl)-2-(2- | 9.21 (d, J = 1.0 Hz, 1H), 8.65 (dd, J = 4.6, 1.5 Hz, 1H), 8.47 |
| | (pyridin-3-yl)-2H- | (dq, J = 8.5, 1.3 Hz, 1H), 8.15 (q, J = 1.0 Hz, 1H), 7.96 (dd, |
| | indazol-5-yl)-2,4- | J = 9.5, 2.0 Hz, 1H), 7.82 (d, J = 9.5 Hz, 1H), 7.66-7.63 (m, |
| | dihydro-3H-1,2,4-triazol- | 1H), 4.06 (s, 3H), 3.72 (dd, J = 9.9, 7.0 Hz, 1H), 2.17-2.12 (m, |
| | 3-one | 1H), 1.40 (d, J = 6.8 Hz, 3H), 0.97 (d, J = 6.8 Hz, 3H), 0.80 |
| | | (d, J = 6.6 Hz, 3H); ESI MS (m/z): 379.25 [M + 1]. |
| 323 | 4-cyclopropyl-5- | ¹H-NMR (400 MHz, DMSO-d₆) δ 9.52 (s, 2H), 9.27-9.26 (m, |
| | methoxy-2-(2- | 2H), 8.13 (t, J = 1.1 Hz, 1H), 7.96 (dd, J = 9.5, 2.0 Hz, 1H), |
| | (pyrimidin-5-yl)-2H- | 7.83 (d, J = 9.3 Hz, 1H), 4.04 (s, 3H), 2.81-2.78 (m, 1H), |
| | indazol-5-yl)-2,4- | 0.95-0.91 (m, 4H); ESI MS (m/z): 350.20 [M + 1]. |
| | dihydro-3H-1,2,4-triazol- | |
| | 3-one | |
| 324 | 4-ethyl-5-methoxy-2-(2- | ¹H-NMR (400 MHz, DMSO-d₆) δ 9.34 (d, J = 2.2 Hz, 1H), |
| | (pyridin-3-yl)-2H- | 9.30 (d, J = 1.0 Hz, 1H), 8.67 (dd, J = 4.8, 1.3 Hz, 1H), 8.50 |
| | indazol-4-yl)-2,4- | (dq, J = 8.3, 1.4 Hz, 1H), 7.72-7.70 (m, 1H), 7.65 (ddd, J = |
| | dihydro-3H-1,2,4-triazol- | 8.3, 4.6, 0.7 Hz, 1H), 7.55 (d, J = 8.8 Hz, 1H), 7.37 (dd, J = |
| | 3-one | 8.7, 7.5 Hz, 1H), 4.19 (s, 3H), 3.65 (q, J = 7.3 Hz, 2H), 1.24 |
| | | (t, J = 7.2 Hz, 3H); ESI MS (m/z): 337.25 [M + 1]. |
| 325 | 2-(2-(5-fluoropyridin-3- | ¹H-NMR (400 MHz, DMSO-d₆) δ 9.28 (s, 1H), 9.25 (s, 1H), |
| | yl)-2H-indazol-5-yl)-5- | 8.69 (d, J = 2.4 Hz, 1H), 8.49 (dt, J = 9.9, 2.3 Hz, 1H), 8.13 |
| | methoxy-4-(2,2,2- | (d, J = 1.0 Hz, 1H), 7.94 (dd, J = 9.4, 2.1 Hz, 1H), 7.86 (d, J = |
| | trifluoroethyl)-2,4- | 9.3 Hz, 1H), 4.50 (q, J = 9.1 Hz, 2H), 4.09 (d, J = 9.8 Hz, 3H) |
| | dihydro-3H-1,2,4-triazol- | ESI MS (m/z): 408.60 [M + 1]. |
| | 3-one | |
| 326 | 5-ethoxy-2-(2- | ¹H-NMR (400 MHz, DMSO-d₆) δ 9.53 (s, 2H), 9.28 (s, 1H), |
| | (pyrimidin-5-yl)-2H- | 8.15 (d, J = 1.2 Hz, 1H), 7.98 (dd, J = 9.4, 2.1 Hz, 1H), 7.85 |
| | indazol-5-yl)-4-(3- | (d, J = 9.5 Hz, 1H), 7.72 (d, J = 8.3 Hz, 2H), 7.63 (d, J = 5.1 |
| | (trifluoromethyl)benzyl)- | Hz, 2H), 4.89 (s, 2H), 4.45 (q, J = 7.1 Hz, 2H), 1.36 (t, J = 7.0 |
| | 2,4-dihydro-3H-1,2,4- | Hz, 3H), 1.18-1.29 (1H); ESI MS (m/z): 481.5 [M + 1]. |
| | triazol-3-one | |
| 327 | 5-ethoxy-2-(2-(pyridin- | ¹H-NMR (400 MHz, DMSO-d₆) δ 9.32 (d, J = 2.2 Hz, 1H), |
| | 3-yl)-2H-indazol-4-yl)-4- | 9.27 (d, J = 1.0 Hz, 1H), 8.67 (dd, J = 4.8, 1.3 Hz, 1H), 8.49 |
| | (3- | (dq, J = 8.3, 1.4 Hz, 1H), 7.75-7.71 (m, 3H), 7.66-7.63 (m, |
| | (trifluoromethyl)benzyl)- | 3H), 7.57 (d, J = 8.6 Hz, 1H), 7.38 (dd, J = 8.7, 7.5 Hz, 1H), |
| | 2,4-dihydro-3H-1,2,4- | 4.93 (s, 2H), 4.59 (q, J = 7.0 Hz, 2H), 1.39 (t, J = 7.1 Hz, 3H) |
| | triazol-3-one | ESI MS (m/z): 480.55 [M + 1]. |
| 328 | 5-ethoxy-2-(2-(5- | ¹H-NMR (400 MHz, DMSO-d₆) δ 9.31 (d, J = 1.0 Hz, 1H), |
| | fluoropyridin-3-yl)-2H- | 9.26 (s, 1H), 8.71 (d, J = 2.2 Hz, 1H), 8.55-8.52 (m, 1H), 7.73 |
| | indazol-4-yl)-4-(3- | (t, J = 7.1 Hz, 3H), 7.64 (d, J = 5.6 Hz, 2H), 7.56 (d, J = 8.8 |
| | (trifluoromethyl)benzyl)- | Hz, 1H), 7.40 (dd, J = 8.7, 7.5 Hz, 1H), 4.93 (s, 2H), 4.60 (q, |
| | 2,4-dihydro-3H-1,2,4- | J = 7.0 Hz, 2H), 1.39 (t, J = 7.1 Hz, 3H) |
| | triazol-3-one | ESI MS (m/z): 498.55 [M + 1]. |
| 329 | 5-ethoxy-2-(2- | ¹H-NMR (400 MHz, DMSO-d₆) δ 9.55 (s, 2H), 9.35 (d, J = |
| | (pyrimidin-5-yl)-2H- | 1.0 Hz, 1H), 9.28 (s, 1H), 7.76-7.71 (m, 3H), 7.65-7.63 (m, |
| | indazol-4-yl)-4-(3- | 2H), 7.58 (d, J = 8.8 Hz, 1H), 7.40 (dd, J = 8.7, 7.5 Hz, 1H), |
| | (trifluoromethyl)benzyl)- | 4.93 (s, 2H), 4.61 (q, J = 7.1 Hz, 2H), 1.39 (t, J = 7.1 Hz, 3H) |
| | 2,4-dihydro-3H-1,2,4- | ESI MS (m/z): 482.15 [M + 1]. |
| | triazol-3-one | |
| 330 | 5-ethoxy-2-(2-(pyridin- | ¹H-NMR (400 MHz, DMSO-d₆) δ 9.32 (d, J = 2.4 Hz, 1H), |
| | 3-yl)-2H-indazol-5-yl)-4- | 9.22 (d, J = 1.0 Hz, 1H), 8.65 (dd, J = 4.8, 1.3 Hz, 1H), 8.48- |
| | (3- | 8.45 (m, 1H), 8.14 (t, J = 1.0 Hz, 1H), 7.95 (dd, J = 9.5, 2.0 |
| | (trifluoromethyl)benzyl)- | Hz, 1H), 7.83 (d, J = 9.5 Hz, 1H), 7.73-7.70 (m, 2H), 7.66- |
| | 2,4-dihydro-3H-1,2,4- | 7.63 (m, 3H), 4.89 (s, 2H), 4.45 (q, J = 7.0 Hz, 2H), 1.36 (t, J |
| | triazol-3-one | = 7.1 Hz, 3H); ESI MS (m/z): 481.05 [M + 1]. |
| 331 | 5-ethoxy-2-(2-(pyridin- | ¹H-NMR (400 MHz, DMSO-d₆) δ 9.31 (d, J = 2.4 Hz, 1H), |
| | 3-yl)-2H-indazol-4-yl)-4- | 9.21 (d, J = 0.9 Hz, 1H), 8.65 (dd, J = 4.6, 1.5 Hz, 1H), 8.46 |
| | (4- | (dq, J = 8.3, 1.4 Hz, 1H), 8.14 (q, J = 0.9 Hz, 1H), 7.95 (dd, J |
| | (trifluoromethoxy)benzyl)- | = 9.5, 2.1 Hz, 1H), 7.84-7.81 (m, 1H), 7.66-7.63 (m, 1H), |
| | 2,4-dihydro-3H-1,2,4- | 7.48-7.45 (m, 2H), 7.39-7.37 (m, 2H), 4.81 (s, 2H), 4.45 (q, J |
| | triazol-3-one | = 7.0 Hz, 2H), 1.37 (t, J = 7.0 Hz, 3H) |
| | | ESI MS (m/z): 497.10[M + 1]. |
| 332 | 5-ethoxy-2-(2- | ¹H-NMR (400 MHz, DMSO-d₆) δ 9.52 (s, 2H), 9.28-9.26 (m, |
| | (pyrimidin-5-yl)-2H- | 2H), 8.15 (q, J = 0.9 Hz, 1H), 7.97 (dd, J = 9.5, 2.1 Hz, 1H), |
| | indazol-4-yl)-4-(4- | 7.85 (d, J = 9.5 Hz, 1H), 7.47 (d, J = 8.9 Hz, 2H), 7.38 (d, J = |
| | (trifluoromethoxy)benzyl)- | 7.9 Hz, 2H), 4.81 (s, 2H), 4.45 (q, J = 7.0 Hz, 2H), 1.37 (t, J = |
| | 2,4-dihydro-3H-1,2,4- | 7.0 Hz, 3H); ESI MS (m/z): 497.95[M + 1]. |
| | triazol-3-one | |
| 333 | 5-methoxy-2-(2-(pyridin- | ¹H-NMR (400 MHz, DMSO-d₆) δ 9.34 (d, J = 2.4 Hz, 1H), |
| | 3-yl)-2H-indazol-4-yl)-4- | 9.31 (d, J = 0.9 Hz, 1H), 8.67 (dd, J = 4.7, 1.4 Hz, 1H), 8.50 |
| | (4- | (dq, J = 8.3, 1.4 Hz, 1H), 7.72 (dd, J = 7.3, 0.6 Hz, 1H), 7.66- |
| | (trifluoromethoxy)benzyl)- | 7.63 (m, 1H), 7.57 (d, J = 8.9 Hz, 1H), 7.49-7.47 (m, 2H), |
| | 2,4-dihydro-3H-1,2,4- | 7.40-7.36 (m, 3H), 4.86 (s, 2H), 4.18 (s, 3H) |
| | triazol-3-one | ESI MS (m/z): 482.90[M + 1]. |
| 334 | 5-methoxy-2-(2- | ¹H-NMR (400 MHz, DMSO-d₆) δ 9.56 (s, 2H), 9.39 (d, J = |
| | (pyrimidin-5-yl)-2H- | 0.9 Hz, 1H), 9.28 (s, 1H), 7.75 (d, J = 7.0 Hz, 1H), 7.58 (d, J = |
| | indazol-4-yl)-4-(4- | 8.6 Hz, 1H), 7.48 (dd, J = 9.2, 2.4 Hz, 2H), 7.41-7.37 (m, 3H), |
| | (trifluoromethoxy)benzyl)- | 4.87 (s, 2H), 4.19 (s, 3H); ESI MS (m/z): 483.85[M + 1]. |
| | 2,4-dihydro-3H-1,2,4- | |
| | triazol-3-one | |
| 335 | 5-methoxy-2-(2-(pyridin- | ¹H-NMR (400 MHz, DMSO-d₆) δ 9.32 (d, J = 2.4 Hz, 1H), |
| | 3-yl)-2H-indazol-5-yl)-4- | 9.22 (d, J = 0.9 Hz, 1H), 8.65 (dd, J = 4.6, 1.5 Hz, 1H), 8.47 |
| | (3- | (dq, J = 8.3, 1.3 Hz, 1H), 8.16 (q, J = 0.9 Hz, 1H), 7.96 (dd, |
| | (trifluoromethyl)benzyl)- | J = 9.5, 2.1 Hz, 1H), 7.84 (d, J = 9.5 Hz, 1H), 7.72-7.69 (m, |
| | 2,4-dihydro-3H-1,2,4- | 2H), 7.66-7.62 (m, 3H), 4.90 (s, 2H), 4.06 (s, 3H) |
| | triazol-3-one | ESI MS (m/z): 467.00[M + 1]. |
| 336 | 4-(4-fluorobenzyl)-5- | ¹H-NMR (400 MHz, DMSO-d₆) δ 9.34 (d, J = 2.4 Hz, 1H), |
| | methoxy-2-(2-(pyridin-3- | 9.30 (d, J = 0.9 Hz, 1H), 8.67 (dd, J = 4.6, 1.5 Hz, 1H), 8.50 |
| | yl)-2H-indazol-4-yl)-2,4- | (dq, J = 8.4, 1.3 Hz, 1H), 7.72 (dd, J = 7.3, 0.6 Hz, 1H), 7.66- |
| | dihydro-3H-1,2,4-triazol- | 7.63 (m, 1H), 7.56 (d, J = 8.9 Hz, 1H), 7.42-7.38 (m, 3H), |
| | 3-one | 7.21 (tt, J = 9.3, 2.5 Hz, 2H), 4.81 (s, 2H), 4.18 (s, 3H) |
| | | ESI MS (m/z): 417.00[M + 1]. |
| 337 | 5-ethoxy-4-(4- | ′H-NMR (400 MHz, DMSO-d₆) δ 9.31 (d, J = 2.4 Hz, 1H), |
| | fluorobenzyl)-2-(2- | 9.21 (d, J = 0.9 Hz, 1H), 8.65 (dd, J = 4.6, 1.2 Hz, 1H), 8.46 |
| | (pyridin-3-yl)-2H- | (dq, J = 8.3, 1.4 Hz, 1H), 8.13 (q, J = 0.9 Hz, 1H), 7.94 (dd, J |
| | indazol-5-yl)-2,4- | = 9.3, 2.0 Hz, 1H), 7.82 (d, J = 9.5 Hz, 1H), 7.64 (q, J = 4.2 |
| | dihydro-3H-1,2,4-triazol- | Hz, 1H), 7.41-7.36 (m, 2H), 7.23-7.18 (m, 2H), 4.76 (s, 2H), |
| | 3-one | 4.44 (q, J = 7.0 Hz, 2H), 1.37 (t, J = 7.0 Hz, 3H); ESI MS |
| | | (m/z): 431.30[M + 1]. |
| 338 | 5-ethoxy-2-(2-(5- | ¹H-NMR (400 MHz, DMSO-d₆) δ 9.26-9.24 (m, 2H), 8.68 (d, |
| | fluoropyridin-3-yl)-2H- | J = 2.4 Hz, 1H), 8.49 (dt, J = 10.0, 2.3 Hz, 1H), 8.13 (q, J = |
| | indazol-4-yl)-4-(4- | 0.9 Hz, 1H), 7.96 (dd, J = 9.5, 1.8 Hz, 1H), 7.83 (d, J = 9.5 |
| | (trifluoromethoxy)benzyl)- | Hz, 1H), 7.47 (d, J = 8.9 Hz, 2H), 7.38 (d, J = 8.3 Hz, 2H), |
| | 2,4-dihydro-3H-1,2,4- | 4.81 (s, 2H), 4.45 (q, J = 7.1 Hz, 2H), 1.37 (t, J = 7.0 Hz, 3H) |
| | triazol-3-one | ESI MS (m/z): 515.15[M + 1]. |
| 339 | 4-(4-fluorobenzyl)-5- | ¹H-NMR (400 MHz, DMSO-d₆) δ 10.05 (q, J = 1.3 Hz, 1H), |
| | methoxy-2-(2- | 9.42-9.41 (m, 2H), 8.34 (q, J = 2.9 Hz, 1H), 8.15 (q, J = 0.9 |
| | (pyrimidin-5-yl)-2H- | Hz, 1H), 8.01 (dd, J = 9.5, 2.0 Hz, 1H), 7.86 (d, J = 9.5 Hz, |
| | indazol-5-yl)-2,4- | 1H), 7.39 (dd, J = 8.8, 5.4 Hz, 2H), 7.22-7.18 (m, 2H), 4.77 (s, |
| | dihydro-3H-1,2,4-triazol- | 2H), 4.06 (s, 3H); ESI MS (m/z): 418.15[M + 1]. |
| | 3-one | |
| 340 | 4-(4-fluorobenzyl)-2-(2- | ¹H-NMR (400 MHz, DMSO-d₆) δ 9.35 (d, J = 0.7 Hz, 1H), |
| | (5-fluoropyridin-3-yl)- | 9.27 (s, 1H), 8.71 (d, J = 2.7 Hz, 1H), 8.54 (dt, J = 9.9, 2.3 Hz, |
| | 2H-indazol-4-yl)-5- | 1H), 7.72 (d, J = 7.1 Hz, 1H), 7.56 (d, J = 8.8 Hz, 1H), 7.41- |
| | methoxy-2,4-dihydro- | 7.37 (m, 3H), 7.21 (t, J = 8.9 Hz, 2H), 4.81 (s, 2H), 4.18 (s, |
| | 3H-1,2,4-triazol-3-one | 3H); ESI MS (m/z): 435.10[M + 1]. |
| 341 | 5-ethoxy-4-(4- | ¹H-NMR (400 MHz, DMSO-d₆) δ 10.05 (dd, J = 2.9, 1.0 Hz, |
| | fluorobenzyl)-2-(2- | 1H), 9.42-9.41 (m, 2H), 8.33 (q, J = 2.9 Hz, 1H), 8.13 (q, J = |
| | (pyrimidin-5-yl)-2H- | 0.9 Hz, 1H), 8.00 (dd, J = 9.5, 2.0 Hz, 1H), 7.85 (d, J = 9.3 |
| | indazol-5-yl)-2,4- | Hz, 1H), 7.41-7.37 (m, 2H), 7.23-7.18 (m, 2H), 4.76 (s, 2H), |
| | dihydro-3H-1,2,4-triazol- | 4.45 (q, J = 7.0 Hz, 2H), 1.37 (t, J = 7.1 Hz, 3H); ESI MS |
| | 3-one | (m/z): 432.35[M + 1]. |
| 342 | 5-ethoxy-4-(4- | ¹H-NMR (400 MHz, DMSO-d₆) δ 9.25-9.24 (m, 2H), 8.68 (d, |
| | fluorobenzyl)-2-(2-(5- | J = 2.7 Hz, 1H), 8.49 (dt, J = 10.0, 2.3 Hz, 1H), 8.12 (q, J = |
| | fluoropyridin-3-yl)-2H- | 0.9 Hz, 1H), 7.96 (dd, J = 9.5, 2.0 Hz, 1H), 7.82 (d, J = 9.5 |
| | indazol-5-yl)-2,4- | Hz, 1H), 7.41-7.37 (m, 2H), 7.23-7.18 (m, 2H), 4.76 (s, 2H), |
| | dihydro-3H-1,2,4-triazol- | 4.44 (q, J = 7.0 Hz, 2H), 1.37 (t, J = 7.1 Hz, 3H); ESI MS |
| | 3-one | (m/z): 449.20[M + 1]. |
| 343 | 5-ethoxy-4-(4- | ¹H-NMR (400 MHz, DMSO-d₆) δ 9.32 (d, J = 2.2 Hz, 1H), |
| | fluorobenzyl)-2-(2- | 9.26 (d, J = 1.0 Hz, 1H), 8.67 (dd, J = 4.8, 1.3 Hz, 1H), 8.49 |
| | (pyridin-3-yl)-2H- | (dq, J = 8.3, 1.4 Hz, 1H), 7.72-7.70 (m, 1H), 7.66-7.63 (m, |
| | indazol-4-yl)-2,4- | 1H), 7.56 (d, J = 8.6 Hz, 1H), 7.43-7.35 (m, 3H), 7.24-7.19 |
| | dihydro-3H-1,2,4-triazol- | (m, 2H), 4.80 (s, 2H), 4.58 (q, J = 7.1 Hz, 2H), 1.40 (t, J = 7.1 |
| | 3-one | Hz, 3H); ESI MS (m/z): 431.25[M + 1]. |
| 344 | 5-ethoxy-4-(4- | ¹H-NMR (400 MHz, DMSO-d₆) δ 9.55 (s, 2H), 9.34 (d, J = |
| | fluorobenzyl)-2-(2- | 1.0 Hz, 1H), 9.28 (s, 1H), 7.74 (dd, J = 7.5, 0.6 Hz, 1H), 7.57 |
| | (pyrimidin-5-yl)-2H- | (d, J = 8.8 Hz, 1H), 7.43-7.38 (m, 3H), 7.24-7.19 (m, 2H), |
| | indazol-4-yl)-2,4- | 4.80 (s, 2H), 4.60 (q, J = 7.0 Hz, 2H), 1.40 (t, J = 7.1 Hz, 3H) |
| | dihydro-3H-1,2,4-triazol- | ESI MS (m/z): 432.40[M + 1]. |
| | 3-one | |
| 345 | 5-ethoxy-4-(4- | ¹H-NMR (400 MHz, DMSO-d₆) δ 9.30 (d, J = 1.0 Hz, 1H), |
| | fluorobenzyl)-2-(2-(5- | 9.26 (s, 1H), 8.71 (d, J = 2.2 Hz, 1H), 8.53 (dt, J = 9.9, 2.4 Hz, |
| | fluoropyridin-3-yl)-2H- | 1H), 7.72-7.70 (m, 1H), 7.56 (d, J = 8.6 Hz, 1H), 7.43-7.37 |
| | indazol-4-yl)-2,4- | (m, 3H), 7.25-7.19 (m, 2H), 4.80 (s, 2H), 4.59 (q, J = 7.1 Hz, |
| | dihydro-3H-1,2,4-triazol- | 2H), 1.40 (t, J = 7.1 Hz, 3H); ESI MS (m/z): 449.04[M + 1]. |
| | 3-one | |
| 346 | 2-(2-(5-fluoropyridin-3- | ¹H-NMR (400 MHz, DMSO-d₆) δ 9.26-9.25 (m, 2H), 8.69 (d, |
| | yl)-2H-indazol-5-yl)-5- | J = 2.7 Hz, 1H), 8.49 (dt, J = 10.0, 2.3 Hz, 1H), 8.15 (q, J = |
| | methoxy-4-(3- | 1.0 Hz, 1H), 7.98 (dd, J = 9.5, 2.0 Hz, 1H), 7.84 (d, J = 9.5 |
| | (trifluoromethyl)benzyl)- | Hz, 1H), 7.72-7.69 (m, 2H), 7.65-7.62 (m, 2H), 4.90 (s, 2H), |
| | 2,4-dihydro-3H-1,2,4- | 4.06 (s, 3H); ESI MS (m/z): 485.25[M + 1]. |
| | triazol-3-one | |
| 347 | 2-(3-chloro-2-(pyridin-3- | ¹H-NMR (400 MHz, DMSO-d₆) δ 9.00 (d, J = 2.4 Hz, 1H), |
| | yl)-2H-indazol-5-yl)-5- | 8.81 (dd, J = 4.9, 1.5 Hz, 1H), 8.26 (dq, J = 8.2, 1.3 Hz, 1H), |
| | methoxy-4-methyl-2,4- | 7.87 (dd, J = 9.3, 0.7 Hz, 1H), 7.72 (ddd, J = 8.2, 4.8, 0.7 Hz, |
| | dihydro-3H-1,2,4-triazol- | 1H), 7.64 (q, J = 1.0 Hz, 1H), 7.47 (dd, J = 9.3, 2.0 Hz, 1H), |
| | 3-one | 3.04 (d, J = 4.4 Hz, 3H), 3.01 (s, 3H); ESI MS (m/z): 356.85 |
| | | [M + 1]. |
| 348 | 4-(4-chlorobenzyl)-5- | ¹H-NMR (400 MHz, DMSO-d₆) δ 10.05 (q, J = 1.2 Hz, 1H), |
| | methoxy-2-(2- | 9.42-9.41 (m, 2H), 8.34 (q, J = 2.9 Hz, 1H), 8.15 (d, J = 1.2 |
| | (pyrimidin-5-yl)-2H- | Hz, 1H), 8.01 (dd, J = 9.5, 2.1 Hz, 1H), 7.86 (d, J = 9.5 Hz, |
| | indazol-5-yl)-2,4- | 1H), 7.45-7.42 (m, 2H), 7.36 (d, J = 8.6 Hz, 2H), 4.78 (s, 2H), |
| | dihydro-3H-1,2,4-triazol- | 4.05 (s, 3H); ESI MS (m/z): 434.15[M + 1]. |
| | 3-one | |
| 349 | 4-(4-chlorobenzyl)-5- | ¹H-NMR (400 MHz, DMSO-d₆) δ 9.56 (s, 2H), 9.39 (d, J = |
| | methoxy-2-(2- | 0.9 Hz, 1H), 9.28 (s, 1H), 7.75 (d, J = 7.0 Hz, 1H), 7.58 (d, J = |
| | (pyrimidin-5-yl)-2H- | 8.9 Hz, 1H), 7.46-7.36 (m, 5H), 4.83 (s, 2H), 4.19 (s, 3H) |
| | indazol-4-yl)-2,4- | ESI MS (m/z): 434.20[M + 1]. |
| | dihydro-3H-1,2,4-triazol- | |
| | 3-one | |
| 350 | 4-(4-chlorobenzyl)-5- | ¹H-NMR (400 MHz, DMSO-d₆) δ 9.55 (s, 2H), 9.34 (d, J = |
| | ethoxy-2-(2-(pyrimidin- | 0.9 Hz, 1H), 9.28 (s, 1H), 7.74 (d, J = 7.0 Hz, 1H), 7.57 (d, J = |
| | 5-yl)-2H-indazol-4-yl)- | 8.9 Hz, 1H), 7.47-7.44 (m, 2H), 7.42-7.36 (m, 3H), 4.83-4.77 |
| | 2,4-dihydro-3H-1,2,4- | (m, 2H), 4.60 (q, J = 7.0 Hz, 2H), 1.40 (t, J = 7.0 Hz, 3H) |
| | triazol-3-one | ESI MS (m/z): 447.95[M + 1]. |
| 351 | 4-(4-chlorobenzyl)-5- | ¹H-NMR (400 MHz, DMSO-d₆) δ 9.30 (d, J = 0.6 Hz, 1H), |
| | ethoxy-2-(2-(5- | 9.26 (s, 1H), 8.71 (d, J = 2.4 Hz, 1H), 8.53 (dt, J = 10.0, 2.4 |
| | fluoropyridin-3-yl)-2H- | Hz, 1H), 7.71 (d, J = 7.0 Hz, 1H), 7.56 (d, J = 8.9 Hz, 1H), |
| | indazol-4-yl)-2,4- | 7.47-7.44 (m, 2H), 7.41-7.37 (m, 3H), 4.81 (s, 2H), 4.59 (q, |
| | dihydro-3H-1,2,4-triazol- | J = 7.0 Hz, 2H), 1.40 (t, J = 7.0 Hz, 3H); ESI MS (m/z): |
| | 3-one | 464.95[M + 1]. |
| 352 | 5-methoxy-2-(2-(pyridin- | ¹H-NMR (400 MHz, DMSO-d₆) δ 9.34 (d, J = 2.4 Hz, 1H), |
| | 3-yl)-2H-indazol-4-yl)-4- | 9.31 (d, J = 0.6 Hz, 1H), 8.67 (dd, J = 4.7, 1.4 Hz, 1H), 8.50 |
| | (3- | (dq, J = 8.3, 1.4 Hz, 1H), 7.74-7.70 (m, 3H), 7.66-7.63 (m, |
| | (trifluoromethyl)benzyl)- | 3H), 7.57 (d, J = 8.9 Hz, 1H), 7.38 (dd, J = 8.7, 7.5 Hz, 1H), |
| | 2,4-dihydro-3H-1,2,4- | 4.94 (s, 2H), 4.18 (s, 3H); ESI MS (m/z): 467.00[M + 1]. |
| | triazol-3-one | |
| 353 | 2-(2-(5-fluoropyridin-3- | ¹H-NMR (400 MHz, DMSO-d₆) δ 9.35 (d, J = 0.9 Hz, 1H), |
| | yl)-2H-indazol-4-yl)-5- | 9.28 (s, 1H), 8.71 (d, J = 2.4 Hz, 1H), 8.55 (dt, J = 10.0, 2.4 |
| | methoxy-4-(4- | Hz, 1H), 7.72 (d, J = 6.7 Hz, 1H), 7.57 (d, J = 8.6 Hz, 1H), |
| | (trifluoromethoxy)benzyl)- | 7.48 (d, J = 8.9 Hz, 2H), 7.40 (t, J = 8.1 Hz, 3H), 4.87 (s, 2H), |
| | 2,4-dihydro-3H-1,2,4- | 4.19 (s, 3H); ESI MS (m/z): 501.20[M + 1]. |
| | triazol-3-one | |
| 354 | 4-(4-chlorobenzyl)-5- | ¹H-NMR (400 MHz, DMSO-d₆) δ 9.32 (d, J = 2.4 Hz, 1H), |
| | methoxy-2-(2-(pyridin-3- | 9.22 (d, J = 0.9 Hz, 1H), 8.65 (dd, J = 4.7, 1.4 Hz, 1H), 8.47 |
| | yl)-2H-indazol-5-yl)-2,4- | (dq, J = 8.3, 1.4 Hz, 1H), 8.15 (q, J = 0.9 Hz, 1H), 7.96 (dd, |
| | dihydro-3H-1,2,4-triazol- | J = 9.5, 2.1 Hz, 1H), 7.83 (d, J = 9.5 Hz, 1H), 7.66-7.63 (m, |
| | 3-one | 1H), 7.45-7.42 (m, 2H), 7.37-7.34 (m, 2H), 4.78 (s, 2H), 4.05 |
| | | (s, 3H); ESI MS (m/z): 433.10[M + 1]. |
| 355 | 4-(2-fluorobenzyl)-2-(2- | ¹H-NMR (400 MHz, DMSO-d₆) δ 9.26-9.25 (m, 2H), 8.69 (d, |
| | (5-fluoropyridin-3-yl)- | J = 2.7 Hz, 1H), 8.49 (dt, J = 9.9, 2.3 Hz, 1H), 8.14 (t, J = 1.0 |
| | 2H-indazol-5-yl)-5- | Hz, 1H), 7.97 (dd, J = 9.5, 2.0 Hz, 1H), 7.84 (d, J = 9.5 Hz, |
| | methoxy-2,4-dihydro- | 1H), 7.41-7.33 (m, 2H), 7.26-7.19 (m, 2H), 4.84 (s, 2H), 4.05 |
| | 3H-1,2,4-triazol-3-one | (s, 3H); ESI MS (m/z): 434.90[M + 1]. |
| 356 | 4-(2-fluorobenzyl)-5- | ¹H-NMR (400 MHz, DMSO-d₆) δ 9.34 (d, J = 2.7 Hz, 1H), |
| | methoxy-2-(2-(pyridin-3- | 9.31 (d, J = 0.7 Hz, 1H), 8.67 (dd, J = 4.6, 1.2 Hz, 1H), 8.50 |
| | yl)-2H-indazol-4-yl)-2,4- | (dq, J = 8.3, 1.4 Hz, 1H), 7.72 (d, J = 7.3 Hz, 1H), 7.64 (dd, |
| | dihydro-3H-1,2,4-triazol- | J = 8.1, 4.4 Hz, 1H), 7.56 (d, J = 8.6 Hz, 1H), 7.41-7.35 (m, |
| | 3-one | 3H), 7.27-7.20 (m, 2H), 4.88 (s, 2H), 4.17 (s, 3H) |
| | | ESI MS (m/z): 416.90[M + 1]. |
| 357 | 4-(2-fluorobenzyl)-5- | ¹H-NMR (400 MHz, DMSO-d₆) δ 9.56 (s, 2H), 9.39 (d, J = |
| | methoxy-2-(2- | 1.0 Hz, 1H), 9.28 (s, 1H), 7.75 (d, J = 6.8 Hz, 1H), 7.57 (d, J = |
| | (pyrimidin-5-yl)-2H- | 8.8 Hz, 1H), 7.42-7.35 (m, 3H), 7.26-7.20 (m, 2H), 4.88 (s, |
| | indazol-4-yl)-2,4- | 2H), 4.18 (s, 3H); ESI MS (m/z): 417.95[M + 1]. |
| | dihydro-3H-1,2,4-triazol- | |
| | 3-one | |
| 358 | 5-ethoxy-4-(2- | ¹H-NMR (400 MHz, DMSO-d₆) δ 9.31 (d, J = 2.4 Hz, 1H), |
| | fluorobenzyl)-2-(2- | 9.21 (d, J = 0.7 Hz, 1H), 8.65 (dd, J = 4.6, 1.5 Hz, 1H), 8.47 |
| | (pyridin-3-yl)-2H- | (dq, J = 8.3, 1.4 Hz, 1H), 8.13 (q, J = 0.9 Hz, 1H), 7.94 (dd, |
| | indazol-5-yl)-2,4- | J = 9.4, 2.1 Hz, 1H), 7.82 (d, J = 9.5 Hz, 1H), 7.64 (dd, J = 8.3, |
| | dihydro-3H-1,2,4-triazol- | 4.9 Hz, 1H), 7.41-7.35 (m, 2H), 7.26-7.19 (m, 2H), 4.83 (s, |
| | 3-one | 2H), 4.43 (q, J = 7.0 Hz, 2H), 1.34 (t, J = 7.1 Hz, 3H) |
| | | ESI MS (m/z): 430.90[M + 1]. |
| 359 | 5-ethoxy-4-(2- | ¹H-NMR (400 MHz, DMSO-d₆) δ 9.32 (d, J = 2.4 Hz, 1H), |
| | fluorobenzyl)-2-(2- | 9.26 (d, J = 0.7 Hz, 1H), 8.67 (dd, J = 4.6, 1.2 Hz, 1H), 8.49 |
| | (pyridin-3-yl)-2H- | (dq, J = 8.3, 1.4 Hz, 1H), 7.71 (d, J = 6.8 Hz, 1H), 7.64 (q, J = |
| | indazol-4-yl)-2,4- | 4.2 Hz, 1H), 7.56 (d, J = 8.8 Hz, 1H), 7.42-7.35 (m, 3H), 7.27- |
| | dihydro-3H-1,2,4-triazol- | 7.20 (m, 2H), 4.87 (s, 2H), 4.57 (q, J = 7.1 Hz, 2H), 1.37 (t, |
| | 3-one | J = 7.1 Hz, 3H); ESI MS (m/z): 430.90[M + 1]. |
| 360 | 5-ethoxy-4-(2- | ¹H-NMR (400 MHz, DMSO-d₆) δ 9.55 (s, 2H), 9.35 (d, J = |
| | fluorobenzyl)-2-(2- | 0.7 Hz, 1H), 9.28 (s, 1H), 7.74 (d, J = 7.1 Hz, 1H), 7.57 (d, J = |
| | (pyrimidin-5-yl)-2H- | 8.8 Hz, 1H), 7.42-7.37 (m, 3H), 7.27-7.20 (m, 2H), 4.87 (s, |
| | indazol-4-yl)-2,4- | 2H), 4.59 (q, J = 7.0 Hz, 2H), 1.37 (t, J = 7.0 Hz, 3H) |
| | dihydro-3H-1,2,4-triazol- | ESI MS (m/z): 431.95[M + 1]. |
| | 3-one | |
| 361 | 5-ethoxy-4-(2- | ¹H-NMR (400 MHz, DMSO-d₆) δ 9.25-9.24 (m, 2H), 8.68 (d, |
| | fluorobenzyl)-2-(2-(5- | J = 2.4 Hz, 1H), 8.49 (dt, J = 10.0, 2.3 Hz, 1H), 8.13 (d, J = |
| | fluoropyridin-3-yl)-2H- | 1.2 Hz, 1H), 7.96 (dd, J = 9.5, 2.0 Hz, 1H), 7.83 (d, J = 9.5 |
| | indazol-5-yl)-2,4- | Hz, 1H), 7.41-7.35 (m, 2H), 7.26-7.19 (m, 2H), 4.83 (s, 2H), |
| | dihydro-3H-1,2,4-triazol- | 4.43 (q, J = 7.0 Hz, 2H), 1.34 (t, J = 7.0 Hz, 3H) |
| | 3-one | ESI MS (m/z): 449.25[M + 1]. |
| 362 | 4-(3-fluorobenzyl)-2-(2- | ¹H-NMR (400 MHz, DMSO-d₆) δ 9.20-9.32 (2H), 8.63-8.75 |
| | (5-fluoropyridin-3-yl)- | (1H), 8.41-8.55 (1H), 8.09-8.20 (1H), 7.94-8.03 (1H), 7.79- |
| | 2H-indazol-5-yl)-5- | 7.89 (1H), 7.36-7.48 (1H), 7.09-7.23 (3H), 4.76-4.85 (2H), |
| | methoxy-2,4-dihydro- | 4.02-4.09 (3H); ESI MS (m/z): 435.25[M + 1]. |
| | 3H-1,2,4-triazol-3-one | |
| 363 | 4-(3-fluorobenzyl)-5- | ¹H-NMR (400 MHz, DMSO-d₆) δ 9.56 (s, 2H), 9.40 (s, 1H), |
| | methoxy-2-(2- | 9.28 (s, 1H), 7.75 (d, J = 7.1 Hz, 1H), 7.58 (d, J = 8.8 Hz, 1H), |
| | (pyrimidin-5-yl)-2H- | 7.46-7.39 (m, 2H), 7.19-7.14 (m, 3H), 4.85 (s, 2H), 4.19 (s, |
| | indazol-4-yl)-2,4- | 3H); ESI MS (m/z): 418.15[M + 1]. |
| | dihydro-3H-1,2,4-triazol- | |
| | 3-one | |
| 364 | 5-ethoxy-4-(3- | ¹H-NMR (400 MHz, DMSO-d₆) δ 9.25 (d, J = 5.9 Hz, 2H), |
| | fluorobenzyl)-2-(2-(5- | 8.69 (d, J = 2.4 Hz, 1H), 8.49 (dt, J = 9.9, 2.3 Hz, 1H), 8.14 |
| | fluoropyridin-3-yl)-2H- | (d, J = 1.2 Hz, 1H), 7.97 (dd, J = 9.5, 2.0 Hz, 1H), 7.83 (d, J = |
| | indazol-5-yl)-2,4- | 9.5 Hz, 1H), 7.43 (dd, J = 14.1, 8.2 Hz, 1H), 7.18-7.14 (m, |
| | dihydro-3H-1,2,4-triazol- | 3H), 4.80 (s, 2H), 4.45 (q, J = 7.0 Hz, 2H), 1.37 (t, J = 7.1 Hz, |
| | 3-one | 3H); ESI MS (m/z): 449.15[M + 1]. |
| 365 | 4-(3-fluorobenzyl)-5- | ¹H-NMR (400 MHz, DMSO-d₆) δ 9.34 (d, J = 2.4 Hz, 1H), |
| | methoxy-2-(2-(pyridin-3- | 9.32 (d, J = 0.7 Hz, 1H), 8.67 (dd, J = 4.6, 1.5 Hz, 1H), 8.51 |
| | yl)-2H-indazol-4-yl)-2,4- | (dq, J = 8.3, 1.3 Hz, 1H), 7.72 (d, J = 6.8 Hz, 1H), 7.65 (dd, |
| | dihydro-3H-1,2,4-triazol- | J = 8.1, 4.4 Hz, 1H), 7.57 (d, J = 8.6 Hz, 1H), 7.46-7.36 (m, |
| | 3-one | 2H), 7.19-7.14 (m, 3H), 4.85 (s, 2H), 4.18 (s, 3H) |
| | | ESI MS (m/z): 417.05[M + 1]. |
| 366 | 5-ethoxy-4-(3- | ¹H-NMR (400 MHz, DMSO-d₆) δ 9.33 (d, J = 2.2 Hz, 1H), |
| | fluorobenzyl)-2-(2- | 9.27 (d, J = 0.7 Hz, 1H), 8.67 (dd, J = 4.6, 1.5 Hz, 1H), 8.49 |
| | (pyridin-3-yl)-2H- | (dq, J = 8.3, 1.4 Hz, 1H), 7.71 (d, J = 6.8 Hz, 1H), 7.65 (q, J = |
| | indazol-4-yl)-2,4- | 4.2 Hz, 1H), 7.56 (d, J = 8.8 Hz, 1H), 7.46-7.36 (m, 2H), 7.19- |
| | dihydro-3H-1,2,4-triazol- | 7.14 (m, 3H), 4.84 (s, 2H), 4.59 (q, J = 7.0 Hz, 2H), 1.40 (t, |
| | 3-one | J = 7.1 Hz, 3H); ESI MS (m/z): 431.00[M + 1]. |
| 367 | 4-(2-chlorobenzyl)-5- | ¹H-NMR (400 MHz, DMSO-d₆) δ 9.32 (d, J = 2.4 Hz, 1H), |
| | methoxy-2-(2-(pyridin-3- | 9.22 (d, J = 0.9 Hz, 1H), 8.65 (dd, J = 4.7, 1.4 Hz, 1H), 8.47 |
| | yl)-2H-indazol-5-yl)-2,4- | (dq, J = 8.4, 1.3 Hz, 1H), 8.17 (t, J = 0.9 Hz, 1H), 7.98 (dd, J = |
| | dihydro-3H-1,2,4-triazol- | 9.3, 2.0 Hz, 1H), 7.84 (d, J = 9.5 Hz, 1H), 7.66-7.63 (m, 1H), |
| | 3-one | 7.52-7.50 (m, 1H), 7.38-7.34 (m, 2H), 7.27-7.25 (m, 1H), 4.87 |
| | | (s, 2H), 4.04 (s, 3H); ESI MS (m/z): 433.05[M + 1]. |
| 368 | 4-(3,4-dichlorobenzyl)-5- | ¹H-NMR (400 MHz, DMSO-d₆) δ 9.32 (d, J = 2.4 Hz, 1H), |
| | methoxy-2-(2-(pyridin-3- | 9.22 (s, 1H), 8.65 (dd, J = 4.9, 1.2 Hz, 1H), 8.48-8.46 (m, 1H), |
| | yl)-2H-indazol-5-yl)-2,4- | 8.15 (d, J = 1.5 Hz, 1H), 7.96 (dd, J = 9.5, 1.8 Hz, 1H), 7.84 |
| | dihydro-3H-1,2,4-triazol- | (d, J = 9.5 Hz, 1H), 7.65 (q, J = 4.2 Hz, 3H), 7.32 (dd, J = 8.3, |
| | 3-one | 1.8 Hz, 1H), 4.80 (s, 2H), 4.06 (s, 3H); ESI MS (m/z): |
| | | 467.00[M + 1]. |
| 369 | 4-(2,4-dichlorobenzyl)-5- | ¹H-NMR (400 MHz, DMSO-d₆) δ 9.32 (d, J = 2.1 Hz, 1H), |
| | methoxy-2-(2-(pyridin-3- | 9.22 (d, J = 0.9 Hz, 1H), 8.65 (dd, J = 4.7, 1.4 Hz, 1H), 8.47 |
| | yl)-2H-indazol-5-yl)-2,4- | (dq, J = 8.3, 1.4 Hz, 1H), 8.16 (d, J = 1.2 Hz, 1H), 7.97 (dd, |
| | dihydro-3H-1,2,4-triazol- | J = 9.3, 2.0 Hz, 1H), 7.84 (d, J = 9.5 Hz, 1H), 7.69 (d, J = 2.1 |
| | 3-one | Hz, 1H), 7.67-7.63 (m, 1H), 7.45 (dd, J = 8.6, 2.1 Hz, 1H), |
| | | 7.33 (d, J = 8.6 Hz, 1H), 4.85 (s, 2H), 4.04 (s, 3H) |
| | | ESI MS (m/z): 467.00[M + 1]. |
| 370 | 5-methoxy-4-methyl-2- | ¹H-NMR (400 MHz, DMSO-d₆) δ 9.55 (s, 2H), 9.34-9.30 (m, |
| | (2-(pyrimidin-5-yl)-2H- | 3H), 8.06 (d, J = 1.2 Hz, 1H), 4.05 (s, 3H), 3.14 (s, 3H) |
| | pyrazolo[3,4-c]pyridin-5- | ESI MS (m/z): 425.1[M + 1]. |
| | yl)-2,4-dihydro-3H- | |
| | 1,2,4-triazol-3-one | |
| 371 | 2-(2-(5-fluoropyridin-3- | ¹H-NMR (400 MHz, DMSO-d₆) δ 9.40 (d, J = 0.7 Hz, 1H), |
| | yl)-2H-pyrazolo[3,4- | 9.31-9.29 (m, 2H), 8.78 (d, J = 2.2 Hz, 1H), 8.58 (dt, J = 9.8, |
| | c]pyridin-5-yl)-5- | 2.3 Hz, 1H), 8.07 (d, J = 1.2 Hz, 1H), 4.03 (s, 3H), 3.12 (s, |
| | methoxy-4-methyl-2,4- | 3H); ESI MS (m/z): 341.9[M + 1]. |
| | dihydro-3H-1,2,4-triazol- | |
| | 3-one | |
| 374 | 4-(2-chlorobenzyl)-5- | ¹H-NMR (400 MHz, DMSO-d₆) δ 10.05 (d, J = 1.8 Hz, 1H), |
| | methoxy-2-(2- | 9.42 (d, J = 6.1 Hz, 2H), 8.34 (q, J = 3.0 Hz, 1H), 8.16 (d, J = |
| | (pyrimidin-5-yl)-2H- | 1.2 Hz, 1H), 8.03 (dd, J = 9.5, 1.8 Hz, 1H), 7.87 (d, J = 9.5 |
| | indazol-5-yl)-2,4- | Hz, 1H), 7.51 (t, J = 4.6 Hz, 1H), 7.37-7.35 (m, 2H), 7.27 (d, |
| | dihydro-3H-1,2,4-triazol- | J = 4.9 Hz, 1H), 4.87 (s, 2H), 4.04 (s, 3H); ESI MS (m/z): |
| | 3-one | 434.00[M + 1]. |
| 375 | 4-(3,4-dichlorobenzyl)-5- | ¹H-NMR (400 MHz, DMSO-d₆) δ 10.05 (q, J = 1.2 Hz, 1H), |
| | methoxy-2-(2- | 9.43-9.41 (m, 2H), 8.34 (q, J = 2.9 Hz, 1H), 8.15 (d, J = 1.2 |
| | (pyrimidin-5-yl)-2H- | Hz, 1H), 8.02 (dd, J = 9.5, 1.8 Hz, 1H), 7.86 (d, J = 9.5 Hz, |
| | indazol-5-yl)-2,4- | 1H), 7.65-7.63 (m, 2H), 7.32 (dd, J = 8.4, 2.0 Hz, 1H), 4.80 (s, |
| | dihydro-3H-1,2,4-triazol- | 2H), 4.06 (s, 3H); ESI MS (m/z): 468.00[M + 1]. |
| | 3-one | |
| 379 | 4-(4-chlorobenzyl)-5- | ¹H-NMR (400 MHz, DMSO-d₆) δ 9.56 (d, J = 4.9 Hz, 2H), |
| | ethoxy-2-(2-(pyrimidin- | 9.48 (d, J = 0.7 Hz, 1H), 9.29 (s, 1H), 8.36 (dd, J = 9.5, 1.0 |
| | 5-yl)-2H-pyrazolo[4,3- | Hz, 1H), 8.13 (d, J = 9.5 Hz, 1H), 7.45 (dd, J = 6.6, 2.2 Hz, |
| | b]pyridin-5-yl)-2,4- | 2H), 7.38-7.35 (m, 2H), 4.78 (s, 2H), 4.46 (q, J = 7.1 Hz, 2H), |
| | dihydro-3H-1,2,4-triazol- | 1.37 (t, J = 7.1 Hz, 3H); ESI MS (m/z): 449.10[M + 1]. |
| | 3-one | |

### Example 7: Synthesis of (cyclopropylmethyl)(imino)(2-(pyrimidin-5-yl)-2H-indazol-5-yl)-λ⁶-sulfanone 450

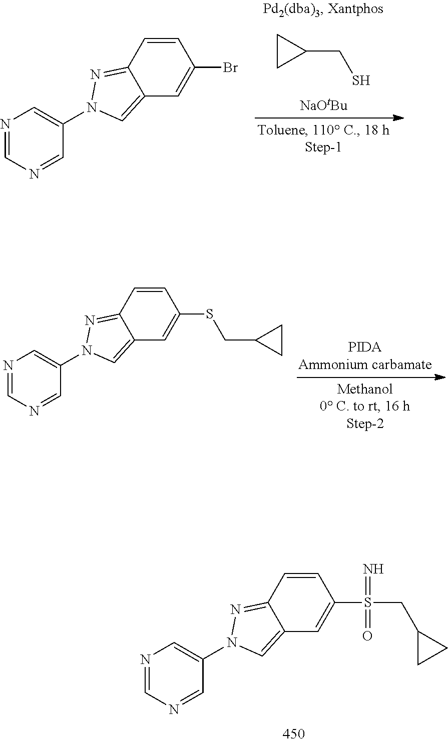 [SMILES: Brc1ccc2nn(-c3cncnc3)cc2c1 ; SCC1CC1 ; c1ncc(-n2cc3cc(SCC4CC4)ccc3n2)cn1 ; "NaOtBu" ; N=S(=O)(CC1CC1)c1ccc2nn(-c3cncnc3)cc2c1]

### Step-1: Synthesis of 5-((cyclopropylmethyl)thio)-2-(pyrimidin-5-yl)-2H-indazole

To a stirred solution of 5-bromo-2-(pyrimidin-5-yl)-2H-indazole (15 g, 54.5 mmol) in toluene (200 mL), Xantphos (9.46 g, 16.36 mmol) and sodium tert-butoxide (10.48 g, 109 mmol) were added under N₂ atmosphere and the reaction mixture was degassed with nitrogen for 15 min. Tris(dibenzylideneacetone)dipalladium (9.99 g, 10.90 mmol) and cyclopropylmethanethiol (5.77 mL, 65.4 mmol) were added and the resulting mixture was heated at 110° C. for 18 h. After completion of the reaction, the reaction mixture was filtered over celite and washed with ethyl acetate. The filtrate was dried over anhydrous sodium sulfate, filtered and concentrated under reduced pressure to obtain a crude semi solid. The crude compound was purified by CombiFlash® chromatography to obtain 5-((cyclopropylmethyl)thio)-2-(pyrimidin-5-yl)-2H-indazole (15 g, 53.1 mmol, 97% yield).

### Step-2: Synthesis of (cyclopropylmethyl)(imino)(2-(pyrimidin-5-yl)-2H-indazol-5-yl)-λ⁶-sulfanone 450

To a stirred solution of 5-((cyclopropylmethyl)thio)-2-(pyrimidin-5-yl)-2H-indazole (15 g, 53.1 mmol) in methanol (230 mL), ammonium carbamate (18.66 g, 239 mmol) was added at 0° C. The reaction mixture was brought to room temperature (25° C.) and stirred for 1 h at this temperature. The reaction mixture was cooled again to 0° C. and (diacetoxyiodo)benzene (PIDA, 37.6 g, 117 mmol) was added into it. The resulting solution was stirred at 25° C. for 16 h. After completion of the reaction methanol was concentrated under reduced pressure and the crude residue was diluted with minimum volumes of water (1×50 mL). This solution was further acidified with concentrated HCl (pH=1-2) and the aqueous layer was extracted with ethyl acetate (3×200 mL) to remove residual non-polar organic impurities. The aqueous layer was then basified with sodium bi-carbonate solution (pH=8) and extracted with chloroform (3×250 mL). The combined organic layers were washed further with brine solution (1×250 mL), dried over anhydrous sodium sulfate, filtered and concentrated under reduced pressure to obtain a crude solid. This crude solid was further triturated with methyl tert-butyl ether to obtain (cyclopropylmethyl)(imino)(2-(pyrimidin-5-yl)-2H-indazol-5-yl)-λ⁶-sulfanone 450 (10 g, 31.9 mmol, 60% yield).

TABLE 7
Representative compounds of the present disclosure were prepared according to
the suitable starting material and methods as described in the Example 7.

| Compd | | |
|---|---|---|
| no. | Compound name | Analytical Data |
| 380 | imino(isopropyl)(2- | ¹H-NMR (400 MHz, DMSO-d₆) δ 9.38 (d, J = 2.4 Hz, 1H), 9.29 |
| | (pyridin-3-yl)-2H- | (d, J = 1.0 Hz, 1H), 8.69 (dd, J = 4.8, 1.3 Hz, 1H), 8.57 (dq, J = |
| | indazol-4-yl)-λ⁶- | 8.3, 1.4 Hz, 1H), 8.07-8.05 (m, 1H), 7.67-7.64 (m, 2H), 7.54 |
| | sulfanone | (dd, J = 8.7, 7.0 Hz, 1H), 4.41 (s, 1H), 3.52-3.45 (m, 1H), 1.18 |
| | | (q, J = 3.3 Hz, 6H); ESI MS (m/z): 300.55 [M + 1]. |
| 381 | imino(isopropyl)(2- | ¹H-NMR (400 MHz, DMSO-d₆) δ 9.48 (d, J = 0.6 Hz, 1H), |
| | (pyridin-3-yl)-2H- | 9.36-9.33 (m, 1H), 8.71-8.68 (m, 1H), 8.50 (dq, J = 8.3, 1.4 Hz, |
| | indazol-5-yl)-λ⁶- | 1H), 8.39 (d, J = 0.9 Hz, 1H), 7.90 (d, J = 9.2 Hz, 1H), 7.72- |
| | sulfanone | 7.66 (m, 2H), 4.16 (s, 1H), 3.29-3.24 (m, 1H), 1.20-1.11 (m, |
| | | 6H); ESI MS (m/z): 301.25 [M + 1]. |
| 384 | tert-butyl(imino)(2- | ¹H-NMR (400 MHz, DMSO-d₆) δ 9.47 (d, J = 0.9 Hz, 1H), 9.33 |
| | (pyridin-3-yl)-2H- | (d, J = 2.4 Hz, 1H), 8.70 (dd, J = 4.7, 1.4 Hz, 1H), 8.50 (dq, J = |
| | indazol-5-yl)-λ⁶- | 8.3, 1.4 Hz, 1H), 8.39 (d, J = 0.6 Hz, 1H), 7.89 (d, J = 9.2 Hz, |
| | sulfanone | 1H), 7.72-7.66 (m, 2H), 1.24 (d, J = 12.8 Hz, 11H) |
| | | ESI MS (m/z): 314.55 [M + 1]. |
| 385 | butyl(imino)(2- | ¹H-NMR (400 MHz, DMSO-d₆) δ 9.39 (s, 1H), 9.32 (d, J = 2.8 |
| | (pyridin-3-yl)-2H- | Hz, 1H), 8.69 (d, J = 3.7 Hz, 1H), 8.48 (d, J = 8.3 Hz, 1H), 8.44 |
| | indazol-5-yl)-λ⁶- | (s, 1H), 7.88 (d, J = 9.2 Hz, 1H), 7.76 (dd, J = 9.0, 1.7 Hz, 1H), |
| | sulfanone | 7.66 (q, J = 4.2 Hz, 1H), 3.92 (d, J = 1.5 Hz, 1H), 3.17 (t, J = 7.8 |
| | | Hz, 2H), 1.56 (dd, J = 15.6, 8.3 Hz, 2H), 1.32 (q, J = 7.4 Hz, |
| | | 2H), 0.82 (t, J = 7.5 Hz, 3H); ESI MS (m/z): 315.2 [M + 1]. |
| 386 | (cyclopropylmeth- | ¹H-NMR (400 MHz, DMSO-d₆) δ 9.43-9.53 (1H), 9.30-9.40 |
| | yl)(imino)(2-(pyridin- | (1H), 8.65-8.74 (1H), 8.47-8.57 (1H), 8.39-8.47 (1H), 7.85-7.94 |
| | 3-yl)-2H-indazol-5- | (1H), 7.73-7.78 (1H), 7.65-7.71 (1H), 4.09-4.26 (1H), 3.07-3.20 |
| | yl)-λ⁶-sulfanone | (2H), 0.73-1.02 (1H), 0.24-0.45 (2H),-0.10-0.14 (2H) |
| | | ESI MS (m/z): 313 [M + 1]. |
| 387 | ethyl(imino)(2- | ¹H-NMR (400 MHz, DMSO-d₆) δ 9.48 (d, J = 0.7 Hz, 1H), 9.34 |
| | (pyridin-3-yl)-2H- | (d, J = 2.4 Hz, 1H), 8.70 (dd, J = 4.8, 1.3 Hz, 1H), 8.51 (dq, J = |
| | indazol-5-yl)-λ⁶- | 8.3, 1.4 Hz, 1H), 8.41 (d, J = 0.7 Hz, 1H), 7.91 (d, J = 9.0 Hz, |
| | sulfanone | 1H), 7.73 (dd, J = 9.0, 1.7 Hz, 1H), 7.68 (dd, J = 8.1, 4.9 Hz, |
| | | 1H), 4.20 (s, 1H), 3.17 (q, J = 7.3 Hz, 2H), 1.08 (t, J = 7.3 Hz, |
| | | 3H); ESI MS (m/z): 286.85 [M + 1]. |
| 393 | (2-(5-fluoropyridin- | ¹H-NMR (400 MHz, DMSO-d₆) δ 9.55 (s, 1H), 9.30 (s, 1H), |
| | 3-yl)-2H- | 9.02 (d, J = 2.4 Hz, 1H), 8.85 (d, J = 2.4 Hz, 1H), 8.78 (d, J = |
| | pyrazolo[3,4- | 2.7 Hz, 1H), 8.61 (dt, J = 9.9, 2.4 Hz, 1H), 4.51 (s, 1H), 3.43- |
| | b]pyridin-5- | 3.36 (m, 1H), 1.19 (dd, J = 6.6, 4.2 Hz, 6H) |
| | yl)(imino)(isopropyl)- | ESI MS (m/z): 319.5 [M + 1]. |
| | λ⁶-sulfanone | |
| 395 | (2-(5-fluoropyridin- | ¹H-NMR (400 MHz, DMSO-d₆) δ 9.52 (d, J = 0.7 Hz, 1H), 9.27 |
| | 3-yl)-2H-indazol-5- | (s, 1H), 8.74 (d, J = 2.4 Hz, 1H), 8.55 (dt, J = 9.8, 2.3 Hz, 1H), |
| | yl)(imino)(isopropyl)- | 8.39 (d, J = 0.7 Hz, 1H), 7.90 (d, J = 9.3 Hz, 1H), 7.72 (dd, J = |
| | λ⁶-sulfanone | 9.3, 1.7 Hz, 1H), 4.18 (s, 1H), 3.26 (t, J = 6.7 Hz, 1H), 1.16 (q, |
| | | J = 3.3 Hz, 6H); ESI MS (m/z): 318.5 [M + 1]. |
| 409 | (cyclopropylmethyl)(2- | ¹H-NMR (400 MHz, DMSO-d₆) δ 9.52 (d, J = 0.7 Hz, 1H), 9.28 |
| | (5- | (s, 1H), 8.74 (d, J = 2.7 Hz, 1H), 8.55 (dt, J = 9.9, 2.3 Hz, 1H), |
| | fluoropyridin-3-yl)- | 8.44 (d, J = 0.7 Hz, 1H), 7.89 (d, J = 9.3 Hz, 1H), 7.77 (dd, J = |
| | 2H-indazol-5- | 9.0, 1.7 Hz, 1H), 4.20 (s, 1H), 3.19-3.10 (m, 2H), 0.94-0.87 (m, |
| | yl)(imino)-λ⁶- | 1H), 0.38-0.34 (m, 2H), 0.04-0.01 (m, 2H); ESI MS (m/z): |
| | sulfanone | 330.5 [M + 1]. |
| 410 | imino(isopropyl)(2- | ′H-NMR (400 MHz, DMSO-d₆) δ 9.56-9.54 (m, 3H), 9.31 (s, |
| | (pyrimidin-5-yl)- | 1H), 8.42 (q, J = 0.8 Hz, 1H), 7.92 (dt, J = 9.2, 0.9 Hz, 1H), 7.73 |
| | 2H-indazol-5-yl)- | (dd, J = 9.2, 1.5 Hz, 1H), 4.18 (s, 1H), 3.28 (d, J = 6.7 Hz, 1H), |
| | λ⁶-sulfanone | 1.18-1.14 (m, 6H); ESI MS (m/z): 301.5 [M + 1]. |
| 450 | (cyclopropylmeth- | ¹H-NMR (400 MHz, DMSO-d₆) δ 9.56-9.53 (m, 3H), 9.31 (s, |
| | yl)(imino)(2- | 1H), 8.46 (d, J = 0.6 Hz, 1H), 7.91 (d, J = 9.2 Hz, 1H), 7.78 (dd, |
| | (pyrimidin-5-yl)- | J = 9.2, 1.5 Hz, 1H), 4.21 (s, 1H), 3.18-3.12 (m, 2H), 0.93-0.86 |
| | 2H-indazol-5-yl)- | (m, 1H), 0.38-0.33 (m, 2H), 0.01 (dd, J = 4.6, 3.1 Hz, 2H) |
| | λ⁶-sulfanone | ESI MS (m/z): 313.9 [M + 1]. |
| 454 | (3-chloro-2- | ′H-NMR (400 MHz, DMSO-d₆) δ 9.03 (d, J = 2.1 Hz, 1H), 8.82 |
| | (pyridin-3-yl)-2H- | (dd, J = 4.6, 1.5 Hz, 1H), 8.32-8.29 (m, 2H), 7.91 (dd, J = 9.2, |
| | indazol-5- | 0.9 Hz, 1H), 7.83 (dd, J = 9.2, 1.5 Hz, 1H), 7.74 (ddd, J = 8.3, |
| | yl)(cyclopropylmeth- | 4.9, 0.6 Hz, 1H), 4.29 (s, 1H), 3.19 (d, J = 7.3 Hz, 2H), 0.91 (s, |
| | yl)(imino)-λ⁶- | 1H), 0.38 (dq, J = 8.0, 1.5 Hz, 2H), 0.05-0.03 (m, 2H) |
| | sulfanone | ESI MS (m/z): 346.9 [M + 1]. |

### Example 8: Synthesis of (cyclobutylmethyl)(2-(5-fluoropyridin-3-yl)-2H-indazol-5-yl)(imino)-λ⁶-sulfanone 468

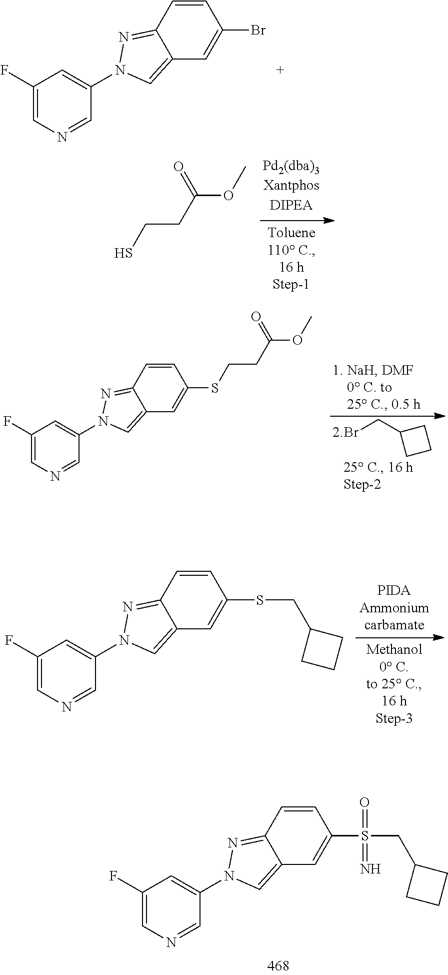

### Step 1: Synthesis of methyl 3-((2-(5-fluoropyridin-3-yl)-2H-indazol-5-yl)thio)propanoate

To a degassed solution of 5-bromo-2-(5-fluoropyridin-3-yl)-2H-indazole (10 g, 34.2 mmol) in toluene (150 mL), Xantphos (9.90 g, 17.12 mmol), N,N-diisopropylethylamine (11.93 mL, 68.5 mmol), tris(dibenzylideneacetone)dipalladium (9.40 g, 10.27 mmol) and methyl 3-mercaptopropanoate (4.55 mL, 41.1 mmol) were added under N₂ atmosphere and the resulting reaction mixture was heated at 110° C. for 16 h. After completion of the reaction, it was cooled to room temperature, filtered over celite and washed with ethyl acetate. The filtrate was dried over anhydrous sodium sulfate, filtered and concentrated under reduced pressure to obtain a crude residue which was purified by CombiFlash® chromatography to obtain the desired methyl 3-((2-(5-fluoropyridin-3-yl)-2H-indazol-5-yl)thio)propanoate (10 g, 30.2 mmol, 88% yield) as a pale-yellow solid.

### Step 2: Synthesis of 5-((cyclobutylmethyl)thio)-2-(5-fluoropyridin-3-yl)-2H-indazole

To a stirred solution of methyl 3-((2-(5-fluoropyridin-3-yl)-2H-indazol-5-yl)thio)propanoate (4 g, 12.07 mmol) in N,N-dimethylformamide (20 mL), sodium hydride (60% dispersed in mineral oil, 0.966 g, 24.14 mmol) was added at 0° C. and stirred for 0.5 h, followed by the addition of (Bromomethyl)cyclobutane (2.71 mL, 24.14 mmol) at the same temperature and the resulting reaction mixture was stirred at 25° C. for 16 h. After completion of the reaction, it was quenched with water (100 mL) and extracted with ethyl acetate (3×100 mL). The combined organic layer was dried over anhydrous sodium sulfate, filtered and concentrated under reduced pressure to obtain the crude compound 5-((cyclobutylmethyl)thio)-2-(5-fluoropyridin-3-yl)-2H-indazole (3.5 g, 11.17 mmol, 93% yield) which was directly used in the next step.

### Step 3: Synthesis of (cyclobutylmethyl)(2-(5-fluoropyridin-3-yl)-2H-indazol-5-yl)(imino)-λ⁶-sulfanone 468

To a stirred solution of 5-((cyclobutylmethyl)thio)-2-(5-fluoropyridin-3-yl)-2H-indazole (3.78 g, 12.06 mmol) in methanol (50 mL), ammonium carbamate (4.24 g, 54.3 mmol) was added at 0° C. and stirred for 0.5 h at 25° C. Iodobenzene diacetate (8.55 g, 26.5 mmol) was added portion wise at 0° C. and the resulting mixture was stirred at 25° C. for 16 h. After completion of the reaction the reaction mixture was concentrated under reduced pressure, neutralized with sodium bicarbonate and extracted with dichloromethane (3×50 mL). The combined organic layer was dried over anhydrous sodium sulfate, filtered and concentrated under reduced pressure to obtain a crude solid. The crude compound was purified by CombiFlash® chromatography to obtain (cyclobutylmethyl)(2-(5-fluoropyridin-3-yl)-2H-indazol-5-yl)(imino)-λ⁶-sulfanone 468 (2.3 g, 6.68 mmol, 55% yield).

TABLE 8
Representative compounds of the present disclosure were prepared according to
the suitable starting material and methods as described in the Example 8.

| Compd | | |
|---|---|---|
| no. | Compound name | Analytical Data |
| 455 | ethyl(imino)(2- | ¹H-NMR (400 MHz, DMSO-d₆) δ 9.56-9.54 (m, 3H), 9.30 (d, |
| | (pyrimidin-5-yl)-2H- | J = 3.4 Hz, 1H), 8.43 (t, J = 0.8 Hz, 1H), 7.93 (d, J = 9.2 Hz, 1H), |
| | indazol-5-yl)-λ⁶- | 7.75 (dd, J = 9.2, 1.8 Hz, 1H), 4.23 (s, 1H), 3.20-3.15 (m, 2H), |
| | sulfanone | 1.08 (t, J = 7.3 Hz, 3H); ESI MS (m/z): 288 [M + 1]. |
| 458 | imino(2-(pyrimidin-5- | ¹H-NMR (400 MHz, DMSO-d₆) δ 9.58-9.56 (m, 3H), 9.32 (d, |
| | yl)-2H-indazol-5- | J = 4.9 Hz, 1H), 8.56 (d, J = 0.6 Hz, 1H), 7.95 (d, J = 9.2 Hz, 1H), |
| | yl)(2,2,2- | 7.84 (dd, J = 9.2, 1.8 Hz, 1H), 5.03 (s, 1H), 4.64 (q, J = 10.1 Hz, |
| | trifluoroethyl)-λ⁶- | 2H); ESI MS (m/z): 342.05 [M + 1]. |
| | sulfanone | |
| 460 | ethyl(2-(5- | ¹H-NMR (400 MHz, DMSO-d₆) δ 9.52 (d, J = 0.7 Hz, 1H), 9.27 |
| | fluoropyridin-3-yl)- | (s, 1H), 8.74 (d, J = 2.4 Hz, 1H), 8.55 (dt, J = 9.9, 2.3 Hz, 1H), |
| | 2H-indazol-5- | 8.41 (d, J = 0.7 Hz, 1H), 7.90 (d, J = 9.0 Hz, 1H), 7.74 (dd, J = |
| | yl)(imino)-λ⁶- | 9.0, 1.7 Hz, 1H), 4.22 (s, 1H), 3.17 (q, J = 7.3 Hz, 2H), 1.08 (t, |
| | sulfanone | J = 7.3 Hz, 3H); ESI MS (m/z): 304.85 [M + 1]. |
| 465 | (cyclobutylmethyl)(imi- | ¹H-NMR (400 MHz, DMSO-d₆) δ 9.52 (s, 2H), 9.46 (d, J = 0.9 |
| | no)(2-(pyrimidin-5-yl)- | Hz, 1H), 9.28 (s, 1H), 8.44 (s, 1H), 7.89 (d, J = 9.2 Hz, 1H), |
| | 2H-indazol-5-yl)-λ⁶- | 7.77 (dd, J = 9.2, 1.8 Hz, 1H), 3.89 (s, 1H), 3.32 (d, J = 7.3 Hz, |
| | sulfanone | 2H), 2.66-2.58 (m, 1H), 1.93-1.86 (m, 2H), 1.82-1.62 (m, 4H) |
| | | ESI MS (m/z): 328.1 [M + 1]. |
| 468 | (cyclobutylmethyl)(2- | ¹H-NMR (400 MHz, DMSO-d₆) δ 9.51 (d, J = 0.5 Hz, 1H), 9.27 |
| | (5-fluoropyridin-3-yl)- | (s, 1H), 8.74 (d, J = 2.4 Hz, 1H), 8.54 (dt, J = 9.9, 2.3 Hz, 1H), |
| | 2H-indazol-5- | 8.39 (d, J = 0.7 Hz, 1H), 7.89 (d, J = 9.0 Hz, 1H), 7.74 (dd, J = |
| | yl)(imino)-λ⁶- | 9.2, 1.6 Hz, 1H), 4.15 (s, 1H), 3.32 (s, 1H), 2.59-2.51 (m, 1H), |
| | sulfanone | 1.87-1.78 (m, 2H), 1.76-1.68 (m, 1H), 1.66-1.57 (m, 3H) |
| | | ESI MS (m/z): 344.9 [M + 1]. |
| 470 | imino(methyl)(2- | ¹H-NMR (400 MHz, DMSO-d₆) δ 9.56 (s, 2H), 9.55-9.54 (m, |
| | (pyrimidin-5-yl)-2H- | 1H), 9.31 (s, 1H), 8.47 (q, J = 0.8 Hz, 1H), 7.93 (dt, J = 9.2, 0.9 |
| | indazol-5-yl)-λ⁶- | Hz, 1H), 7.82 (dd, J = 9.2, 1.8 Hz, 1H), 4.25 (s, 1H), 3.11 (s, |
| | sulfanone | 3H); ESI MS (m/z): 274 [M + 1]. |
| 478 | (2-(5-fluoropyridin-3- | ¹H-NMR (400 MHz, DMSO-d₆) δ 9.52 (d, J = 0.7 Hz, 1H), 9.27 |
| | yl)-2H-indazol-5- | (s, 1H), 8.74 (d, J = 2.7 Hz, 1H), 8.55 (dt, J = 9.9, 2.3 Hz, 1H), |
| | yl)(imino)(propyl)-λ⁶- | 8.41 (q, J = 0.8 Hz, 1H), 7.90 (d, J = 9.3 Hz, 1H), 7.75 (dd, J = |
| | sulfanone | 9.3, 1.7 Hz, 1H), 4.22 (s, 1H), 3.17-3.13 (m, 2H), 1.59-1.50 (m, |
| | | 2H), 0.87 (t, J = 7.5 Hz, 3H); ESI MS (m/z): 319 [M + 1]. |
| 479 | sec-butyl(2-(5- | ¹H-NMR (400 MHz, DMSO-d₆) δ 9.51-9.49 (m, 1H), 9.27 (s, |
| | fluoropyridin-3-yl)- | 1H), 8.74 (d, J = 2.4 Hz, 1H), 8.54 (dt, J = 9.8, 2.3 Hz, 1H), 8.39 |
| | 2H-indazol-5- | (d, J = 1.0 Hz, 1H), 7.90 (d, J = 9.0 Hz, 1H), 7.73-7.69 (m, 1H), |
| | yl)(imino)-λ⁶- | 4.18 (d, J = 8.8 Hz, 1H), 3.05 (qd, J = 6.9, 3.2 Hz, 1H), 1.94- |
| | sulfanone | 1.86 (m, 1H), 1.33-1.24 (m, 1H), 1.14 (dd, J = 9.3, 6.8 Hz, 3H), |
| | | 0.87 (dd, J = 16.4, 7.6 Hz, 3H); ESI MS (m/z): 333 [M + 1]. |
| 480 | (2-(5-fluoropyridin-3- | ¹H-NMR (400 MHz, DMSO-d₆) δ 9.51 (s, 1H), 9.27 (s, 1H), |
| | yl)-2H-indazol-5- | 8.74 (d, J = 2.4 Hz, 1H), 8.54 (d, J = 9.8 Hz, 1H), 8.40 (s, 1H), |
| | yl)(imino)(pentan-3- | 7.90 (d, J = 9.0 Hz, 1H), 7.73 (d, J = 9.0 Hz, 1H), 4.18 (s, 1H), |
| | yl)-λ⁶-sulfanone | 2.92-2.89 (m, 1H), 1.85-1.75 (m, 2H), 1.63-1.52 (m, 2H), 0.92- |
| | | 0.86 (m, 6H); ESI MS (m/z): 347.05 [M + 1]. |
| 482 | (3-fluoropropyl)(2-(5- | ¹H-NMR (400 MHz, DMSO-d₆) δ 9.53 (d, J = 1.0 Hz, 1H), 9.27 |
| | fluoropyridin-3-yl)- | (d, J = 1.0 Hz, 1H), 8.74 (d, J = 2.7 Hz, 1H), 8.55 (dt, J = 9.9, |
| | 2H-indazol-5- | 2.4 Hz, 1H), 8.44 (q, J = 0.8 Hz, 1H), 7.93-7.91 (m, 1H), 7.76 |
| | yl)(imino)-λ⁶- | (dd, J = 9.2, 1.8 Hz, 1H), 4.52 (t, J = 5.9 Hz, 1H), 4.41-4.38 (m, |
| | sulfanone | 2H), 3.27 (dd, J = 9.4, 6.2 Hz, 2H), 1.99-1.87 (m, 2H); ESI MS |
| | | (m/z): 336.85 [M + 1]. |
| 484 | (2-(5-fluoropyridin-3- | ¹H-NMR (400 MHz, DMSO-d₆) δ 9.52 (d, J = 0.7 Hz, 1H), 9.28 |
| | yl)-2H-indazol-5- | (s, 1H), 8.74 (d, J = 2.4 Hz, 1H), 8.55 (dt, J = 9.8, 2.3 Hz, 1H), |
| | yl)(imino)((3- | 8.42 (d, J = 0.7 Hz, 1H), 7.92 (d, J = 9.3 Hz, 1H), 7.78 (dd, J = |
| | methyloxetan-3- | 9.2, 1.8 Hz, 1H), 4.62 (d, J = 5.9 Hz, 1H), 4.46 (d, J = 5.9 Hz, |
| | yl)methyl)-λ⁶- | 1H), 4.30 (s, 1H), 4.12 (d, J = 6.1 Hz, 1H), 4.07 (d, J = 5.9 Hz, |
| | sulfanone | 1H), 3.73 (d, J = 14.2 Hz, 1H), 3.60 (d, J = 14.4 Hz, 1H), 1.47 |
| | | (s, 3H); ESI MS (m/z): 361 [M + 1]. |
| 488 | cyclobutyl(2-(5- | ¹H-NMR (400 MHz, DMSO-d₆) δ 9.51 (td, J = 7.3, 0.7 Hz, 1H), |
| | fluoropyridin-3-yl)- | 9.27 (s, 1H), 8.74 (d, J = 2.4 Hz, 1H), 8.57-8.53 (m, 1H), 8.39 |
| | 2H-indazol-5- | (q, J = 0.8 Hz, 1H), 7.89 (d, J = 9.2 Hz, 1H), 7.70 (ddd, J = 9.1, |
| | yl)(imino)-λ⁶- | 5.1, 1.8 Hz, 1H), 4.19 (d, J = 8.3 Hz, 1H), 4.01-3.93 (m, 1H), |
| | sulfanone | 2.39-2.29 (m, 2H), 2.12-1.93 (m, 2H), 1.90-1.73 (m, 2H) |
| | | ESI MS (m/z): 331 [M + 1]. |
| 554 | cyclobutyl(imino)(2- | ¹H-NMR (400 MHz, DMSO-d₆) δ 9.48 (d, J = 7.3 Hz, 1H), 9.33 |
| | (pyridin-3-yl)-2H- | (d, J = 2.4 Hz, 1H), 8.70 (dd, J = 4.8, 1.3 Hz, 1H), 8.50 (dq, J = |
| | indazol-5-yl)-λ⁶- | 8.3, 1.4 Hz, 1H), 8.39 (s, 1H), 7.89 (d, J = 9.0 Hz, 1H), 7.70- |
| | sulfanone | 7.66 (m, 2H), 4.18 (s, 1H), 4.00-3.92 (m, 1H), 2.39-2.29 (m, |
| | | 2H), 2.11-1.94 (m, 2H), 1.88-1.76 (m, 2H); ESI MS (m/z): 312.9 |
| | | [M + 1] |
| 555 | (cyclohexylmethyl)(imi- | ¹H-NMR (400 MHz, DMSO-d₆) δ 9.47 (d, J = 0.7 Hz, 1H), 9.34 |
| | no)(2-(pyridin-3-yl)- | (d, J = 2.4 Hz, 1H), 8.70 (dd, J = 4.8, 1.3 Hz, 1H), 8.51 (dq, J = |
| | 2H-indazol-5-yl)-λ⁶- | 8.3, 1.4 Hz, 1H), 8.42 (d, J = 1.0 Hz, 1H), 7.90 (d, J = 9.0 Hz, |
| | sulfanone | 1H), 7.75 (dd, J = 9.3, 1.7 Hz, 1H), 7.68 (dd, J = 8.3, 4.9 Hz, |
| | | 1H), 4.20 (s, 1H), 3.13-3.04 (m, 2H), 1.88-1.68 (m, 3H), 1.57- |
| | | 1.49 (m, 3H), 1.19-0.92 (m, 5H); ESI MS (m/z): 355.05 [M + 1] |
| 556 | imino(pentan-3-yl)(2- | ¹H-NMR (400 MHz, DMSO-d₆) δ 9.47 (d, J = 0.6 Hz, 1H), 9.34 |
| | (pyridin-3-yl)-2H- | (d, J = 2.4 Hz, 1H), 8.70 (dd, J = 4.7, 1.4 Hz, 1H), 8.50 (dq, J = |
| | indazol-5-yl)-λ⁶- | 8.4, 1.3 Hz, 1H), 8.40 (d, J = 0.6 Hz, 1H), 7.90 (d, J = 9.2 Hz, |
| | sulfanone | 1H), 7.72 (dd, J = 9.0, 1.7 Hz, 1H), 7.68 (q, J = 4.4 Hz, 1H), 4.16 |
| | | (s, 1H), 2.91-2.88 (m, 1H), 1.85-1.75 (m, 2H), 1.63-1.52 (m, 2H), |
| | | 0.92-0.86 (m, 6H); ESI MS (m/z): 329 [M + 1] |
| 557 | imino(isobutyl)(2- | ¹H-NMR (400 MHz, DMSO-d₆) δ 9.47 (d, J = 0.9 Hz, 1H), 9.34 |
| | (pyridin-3-yl)-2H- | (d, J = 2.4 Hz, 1H), 8.70 (dd, J = 4.7, 1.4 Hz, 1H), 8.51 (dq, J = |
| | indazol-5-yl)-λ⁶- | 8.4, 1.3 Hz, 1H), 8.43 (d, J = 0.9 Hz, 1H), 7.90 (d, J = 9.2 Hz, |
| | sulfanone | 1H), 7.75 (dd, J = 9.2, 1.8 Hz, 1H), 7.69-7.66 (m, 1H), 4.20 (s, |
| | | 1H), 3.14-3.05 (m, 2H), 2.08-2.01 (m, 1H), 0.91 (dd, J = 6.7, 1.5 |
| | | Hz, 6H); ESI MS (m/z): 314.95 [M + 1] |
| 558 | cyclopentyl(imino)(2- | ¹H-NMR (400 MHz, DMSO-d₆) δ 9.46 (d, J = 0.9 Hz, 1H), 9.34 |
| | (pyridin-3-yl)-2H- | (d, J = 2.4 Hz, 1H), 8.70 (dd, J = 4.7, 1.4 Hz, 1H), 8.50 (dq, J = |
| | indazol-5-yl)-λ⁶- | 8.3, 1.4 Hz, 1H), 8.42 (t, J = 0.8 Hz, 1H), 7.90 (d, J = 9.2 Hz, 1H), |
| | sulfanone | 7.74 (dd, J = 9.2, 1.5 Hz, 1H), 7.69-7.66 (m, 1H), 4.10 (s, 1H), |
| | | 3.67-3.63 (m, 1H), 1.93-1.70 (m, 4H), 1.60-1.48 (m, 4H) |
| | | ESI MS (m/z): 326.9 [M + 1] |
| 559 | cycloheptyl(imino)(2- | ¹H-NMR (400 MHz, DMSO-d₆) δ 9.46 (d, J = 1.0 Hz, 1H), 9.33 |
| | (pyridin-3-yl)-2H- | (d, J = 2.2 Hz, 1H), 8.70 (dd, J = 4.8, 1.3 Hz, 1H), 8.50 (dq, J = |
| | indazol-5-yl)-λ⁶- | 8.3, 1.4 Hz, 1H), 8.39 (q, J = 0.8 Hz, 1H), 7.89 (d, J = 9.0 Hz, |
| | sulfanone | 1H), 7.73-7.66 (m, 2H), 4.11 (s, 1H), 3.19-3.12 (m, 1H), 2.10- |
| | | 2.01 (m, 2H), 1.68-1.33 (m, 10H); ESI MS (m/z): 355 [M + 1] |
| 560 | cyclohexyl(imino)(2- | ¹H-NMR (400 MHz, DMSO-d₆) δ 9.47 (d, J = 1.0 Hz, 1H), 9.33 |
| | (pyridin-3-yl)-2H- | (d, J = 2.4 Hz, 1H), 8.70 (dd, J = 4.8, 1.3 Hz, 1H), 8.50 (dq, J = |
| | indazol-5-yl)-λ⁶- | 8.4, 1.3 Hz, 1H), 8.36 (d, J = 0.7 Hz, 1H), 7.89 (d, J = 9.3 Hz, |
| | sulfanone | 1H), 7.70-7.66 (m, 2H), 4.16 (s, 1H), 3.01-2.96 (m, 1H), 1.99- |
| | | 1.91 (m, 2H), 1.72 (d, J = 9.5 Hz, 2H), 1.55 (d, J = 12.2 Hz, 1H), |
| | | 1.31-1.16 (m, 4H), 1.06-0.99 (m, 1H); ESI MS (m/z): 340.95 |
| | | [M + 1] |
| 561 | (2- | ¹H-NMR (400 MHz, DMSO-d₆) δ 9.47 (d, J = 0.9 Hz, 1H), 9.34 |
| | cyclohexylethyl)(imi- | (d, J = 2.4 Hz, 1H), 8.70 (dd, J = 4.7, 1.4 Hz, 1H), 8.51 (dq, J = |
| | no)(2-(pyridin-3-yl)-2H- | 8.3, 1.4 Hz, 1H), 8.41 (q, J = 0.8 Hz, 1H), 7.90 (d, J = 9.2 Hz, |
| | indazol-5-yl)-λ⁶- | 1H), 7.74 (dd, J = 9.2, 1.8 Hz, 1H), 7.69-7.66 (m, 1H), 4.19 (s, |
| | sulfanone | 1H), 3.16 (t, J = 8.3 Hz, 2H), 1.59-1.52 (m, 5H), 1.44-1.38 (m, |
| | | 2H), 1.22 (s, 1H), 1.14-1.02 (m, 3H), 0.81-0.72 (m, 2H); ESI MS |
| | | (m/z): 369.05 [M + 1] |
| 562 | imino(pentan-2-yl)(2- | ¹H-NMR (400 MHz, DMSO-d₆) δ 9.48 (d, J = 0.9 Hz, 1H), 9.34 |
| | (pyridin-3-yl)-2H- | (d, J = 2.4 Hz, 1H), 8.70 (dd, J = 4.7, 1.4 Hz, 1H), 8.51 (dq, J = |
| | indazol-5-yl)-λ⁶- | 8.4, 1.3 Hz, 1H), 8.39 (d, J = 0.9 Hz, 1H), 7.90 (d, J = 9.2 Hz, |
| | sulfanone | 1H), 7.72-7.66 (m, 2H), 4.17 (d, J = 7.0 Hz, 1H), 3.13-3.08 (m, |
| | | 1H), 1.84-1.78 (m, 1H), 1.41-1.12 (m, 6H), 0.81 (q, J = 7.2 Hz, |
| | | 3H); ESI MS (m/z): 328.95 [M + 1] |
| 563 | imino(isopentyl)(2- | ¹H-NMR (400 MHz, DMSO-d₆) δ 9.48 (d, J = 0.7 Hz, 1H), 9.34 |
| | (pyridin-3-yl)-2H- | (d, J = 2.4 Hz, 1H), 8.70 (dd, J = 4.6, 1.2 Hz, 1H), 8.51 (dq, J = |
| | indazol-5-yl)-λ⁶- | 8.3, 1.4 Hz, 1H), 8.41 (t, J = 0.7 Hz, 1H), 7.91 (d, J = 9.3 Hz, 1H), |
| | sulfanone | 7.74 (dd, J = 9.0, 1.7 Hz, 1H), 7.68 (ddd, J = 8.3, 4.8, 0.6 Hz, 1H), |
| | | 4.21 (s, 1H), 3.16 (t, J = 8.2 Hz, 2H), 1.58-1.52 (m, 1H), 1.44- |
| | | 1.38 (m, 2H), 0.79 (d, J = 6.6 Hz, 6H); ESI MS (m/z): 328.95 |
| | | [M + 1] |
| 564 | 4-(2-(pyridin-3-yl)-2H- | ¹H-NMR (400 MHz, DMSO-d₆) δ 9.49 (d, J = 0.7 Hz, 1H), 9.34 |
| | indazole-5-sulfon- | (d, J = 2.2 Hz, 1H), 8.70 (dd, J = 4.8, 1.3 Hz, 1H), 8.51 (dq, J = |
| | imidoyl)butanenitrile | 8.3, 1.4 Hz, 1H), 8.44 (d, J = 1.0 Hz, 1H), 7.93 (d, J = 9.3 Hz, |
| | | 1H), 7.74 (dd, J = 9.2, 1.8 Hz, 1H), 7.68 (dd, J = 8.3, 4.6 Hz, 1H), |
| | | 4.46 (s, 1H), 3.26 (dd, J = 8.7, 5.3 Hz, 2H), 2.61 (t, J = 7.3 Hz, |
| | | 2H), 1.88-1.80 (m, 2H); ESI MS (m/z): 326.05 [M + 1] |
| 569 | imino(2-(pyridin-3-yl)- | ¹H-NMR (400 MHz, DMSO-d₆) δ 9.48 (d, J = 2.7 Hz, 1H), 9.34 |
| | 2H-indazol-5- | (d, J = 2.4 Hz, 1H), 8.70 (d, J = 3.7 Hz, 1H), 8.51 (d, J = 8.3 Hz, |
| | yl)((tetrahydrofuran-2- | 1H), 8.43 (d, J = 9.5 Hz, 1H), 7.88 (dd, J = 9.2, 2.8 Hz, 1H), 7.78- |
| | yl)methyl)-λ⁶- | 7.75 (m, 1H), 7.68 (dd, J = 8.3, 4.6 Hz, 1H), 4.27 (s, 1H), 4.12 (dt, |
| | sulfanone | J = 17.0, 6.4 Hz, 1H), 3.62-3.37 (m, 4H), 1.94-1.87 (m, 1H), 1.79- |
| | | 1.68 (m, 2H), 1.56-1.46 (m, 1H); ESI MS (m/z): 342.9 [M + 1]. |
| 580 | imino(2-(pyridin-3-yl)- | ¹H-NMR (400 MHz, DMSO-d₆) δ 9.51 (d, J = 1.0 Hz, 1H), 9.35 |
| | 2H-indazol-5- | (d, J = 2.2 Hz, 1H), 8.70 (dd, J = 4.6, 1.5 Hz, 1H), 8.53-8.48 (m, |
| | yl)(3,3,3- | 2H), 7.93 (d, J = 9.0 Hz, 1H), 7.79 (dd, J = 9.2, 1.8 Hz, 1H), 7.68 |
| | trifluoropropyl)-λ⁶- | (ddd, J = 8.3, 4.6, 0.7 Hz, 1H), 4.65 (s, 1H), 3.47-3.43 (m, 2H), |
| | sulfanone | 2.62-2.55 (m, 2H); ESI MS (m/z): 354.9 [M + 1]. |
| 602 | imino(neopentyl)(2- | ¹H-NMR (400 MHz, MeOD) δ 9.31 (d, J = 2.0 Hz, 1H), 9.22 (d, |
| | (pyridin-3-yl)-2H- | J = 0.7 Hz, 1H), 8.68 (dd, J = 4.9, 1.5 Hz, 1H), 8.57 (q, J = 0.8 |
| | indazol-5-yl)-λ⁶- | Hz, 1H), 8.52 (dq, J = 8.5, 1.3 Hz, 1H), 7.91 (dt, J = 9.3, 0.9 Hz, |
| | sulfanone | 1H), 7.84 (dd, J = 9.3, 2.0 Hz, 1H), 7.69 (ddd, J = 8.4, 4.8, 0.7 Hz, |
| | | 1H), 3.43-3.36 (m, 2H), 1.15-1.03 (m, 9H); ESI MS (m/z): 329 |
| | | [M + 1]. |
| 609 | imino(propyl)(2- | ¹H-NMR (400 MHz, MeOD) δ 9.31 (s, 1H), 9.23-9.22 (m, 1H), |
| | (pyridin-3-yl)-2H- | 8.67 (d, J = 4.4 Hz, 1H), 8.55-8.51 (m, 2H), 7.91 (d, J = 9.3 Hz, |
| | indazol-5-yl)-λ⁶- | 1H), 7.82 (dd, J = 9.2, 1.8 Hz, 1H), 7.69 (dd, J = 8.3, 4.9 Hz, 1H), |
| | sulfanone | 3.30 (t, J = 1.6 Hz, 2H), 1.75-1.66 (m, 2H), 0.99-0.95 (m, 3H) |
| | | ESI MS (m/z): 301.1 [M + 1]. |
| 619 | imino(isopropyl)(2- | ¹H-NMR (400 MHz, DMSO-d₆) δ 9.78 (d, J = 0.9 Hz, 1H), 9.39 |
| | (pyridin-3-yl)-2H- | (d, J = 2.4 Hz, 1H), 8.74 (dd, J = 4.9, 1.5 Hz, 1H), 8.56 (dq, J = |
| | pyrazolo[4,3- | 8.3, 1.4 Hz, 1H), 8.50 (dd, J = 9.2, 0.9 Hz, 1H), 8.02 (d, J = 8.9 |
| | b]pyridin-5-yl)-λ⁶- | Hz, 1H), 7.71 (ddd, J = 8.3, 4.9, 0.6 Hz, 1H), 4.49 (s, 1H), 3.76- |
| | sulfanone | 3.69 (m, 1H), 1.20 (dd, J = 16.0, 6.9 Hz, 6H); ESI MS (m/z): 302.0 |
| | | [M + 1]. |

### Example 9: Synthesis of (cyclobutylinethyl)(2-(5-fluoropyridin-3-yl)-2H-indazol-5-yl)((6(trifluoromethyl) pyrazin-2-yl)imino)-λ⁶-sulfanone 473

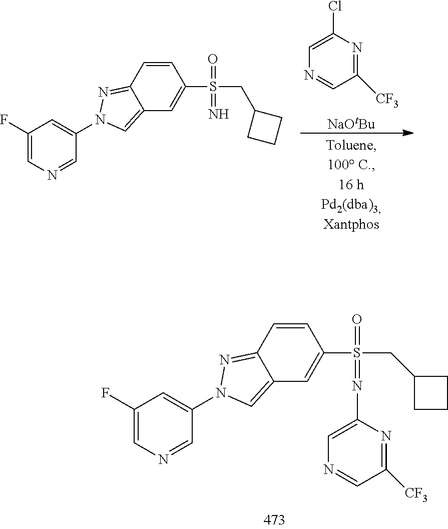 [SMILES: N=S(=O)(CC1CCC1)c1ccc2nn(-c3cncc(F)c3)cc2c1 ; FC(F)(F)c1cncc(Cl)n1 ; O=S(CC1CCC1)(=Nc1cncc(C(F)(F)F)n1)c1ccc2nn(-c3cncc(F)c3)cc2c1 ; "NaOtBu"]

To a degassed solution of (cyclobutylmethyl)(2-(5-fluoropyridin-3-yl)-2H-indazol-5-yl)(imino)-λ⁶-sulfanone (300 mg, 0.871 mmol) in toluene (10 mL), sodium tert-butoxide (184 mg, 1.916 mmol), Xantphos (252 mg, 0.436 mmol), 2-chloro-6-(trifluoromethyl)pyrazine (0.159 mL, 1.307 mmol) and tris(dibenzylideneacetone)dipalladium (239 mg, 0.261 mmol) were added and the resulting reaction mixture was stirred under reflux condition for 16 h. After completion of the reaction, the reaction mixture was filtered through celite and washed with additional volumes of ethyl acetate (3×10 mL). The collective supernatant was concentrated under reduced pressure to obtain a crude residue which was purified by preparative HPLC to obtain the desired (cyclobutylmethyl)(2-(5-fluoropyridin-3-yl)-2H-indazol-5-yl)((6-(trifluoromethyl)pyrazin-2-yl)imino)-λ⁶-sulfanone 473 (130 mg, 0.265 mmol, 30.4% yield).

TABLE 9
Representative compounds of the present disclosure were prepared according to
the suitable starting material and methods as described in the Example 9.

| Comp | | |
|---|---|---|
| no. | Compound name | Analytical Data |
| 448 | (cyclopropylmethyl)(2- | ¹H-NMR (400 MHz, DMSO-d₆) δ 9.54 (d, J = 8.6 Hz, 3H), 9.31 |
| | (pyrimidin-5-yl)-2H- | (s, 1H), 8.56 (d, J = 0.9 Hz, 1H), 7.97 (d, J = 9.5 Hz, 1H), 7.72 |
| | indazol-5-yl)((2,4,5- | (dd, J = 9.3, 1.7 Hz, 1H), 7.41 (td, J = 10.7, 7.9 Hz, 1H), 6.99- |
| | trifluorophenyl)imino)- | 6.92 (m, 1H), 3.66 (dq, J = 23.8, 7.3 Hz, 2H), 1.00-0.94 (m, |
| | λ⁶-sulfanone | 1H), 0.50-0.38 (m, 2H), 0.23-0.02 (m, 2H); ESI MS (m/z): |
| | | 444.05 [M + 1]. |
| 451 | (cyclopropylmethyl)((5- | ¹H-NMR (400 MHz, DMSO-d₆) δ 9.55-9.53 (m, 3H), 9.31 (s, |
| | fluoropyridin-2- | 1H), 8.54 (d, J = 0.9 Hz, 1H), 7.94-7.91 (m, 2H), 7.73 (dd, J = |
| | yl)imino)(2- | 9.2, 1.8 Hz, 1H), 7.49 (td, J = 8.7, 3.1 Hz, 1H), 6.85 (dd, J = 9.0, |
| | (pyrimidin-5-yl)-2H- | 3.8 Hz, 1H), 3.64 (d, J = 7.0 Hz, 2H), 0.91 (dd, J = 11.9, 7.0 Hz, |
| | indazol-5-yl)-λ⁶- | 1H), 0.44-0.34 (m, 2H), 0.10 (q, J = 4.7 Hz, 1H), −0.03 (q, J = |
| | sulfanone | 4.8 Hz, 1H); ESI MS (m/z): 409.05 [M + 1]. |
| 452 | (cyclopropylmethyl)(pyr- | ¹H-NMR (400 MHz, DMSO-d₆) δ 9.54 (d, J = 5.8 Hz, 3H), 9.31 |
| | idin-2-ylimino)(2- | (s, 1H), 8.55 (d, J = 0.6 Hz, 1H), 7.93-7.91 (m, 2H), 7.74 (dd, |
| | (pyrimidin-5-yl)-2H- | J = 9.2, 1.8 Hz, 1H), 7.56-7.51 (m, 1H), 6.82 (d, J = 7.9 Hz, 1H), |
| | indazol-5-yl)-λ⁶- | 6.73 (ddd, J = 7.2, 5.0, 0.9 Hz, 1H), 3.67 (dd, J = 7.2, 1.7 Hz, |
| | sulfanone | 2H), 0.91-0.88 (m, 1H), 0.44-0.32 (m, 2H), 0.11-0.06 (m, 1H), |
| | | 0.05-0.0 (m, 1H); ESI MS (m/z): 391.1 [M + 1]. |
| 453 | (cyclopropylmethyl)((2- | ¹H-NMR (400 MHz, DMSO-d₆) δ 9.53 (d, J = 1.7 Hz, 3H), 9.32 |
| | methoxypyrimidin-5- | (d, J = 7.3 Hz, 1H), 8.57 (s, 1H), 8.15 (d, J = 5.1 Hz, 2H), 7.95 |
| | yl)imino)(2- | (d, J = 9.3 Hz, 1H), 7.74 (dd, J = 9.3, 1.7 Hz, 1H), 3.76-3.71 (m, |
| | (pyrimidin-5-yl)-2H- | 3H), 3.69-3.61 (m, 2H), 1.00 (t, J = 7.6 Hz, 1H), 0.53-0.46 (m, |
| | indazol-5-yl)-λ⁶- | 1H), 0.43-0.36 (m, 1H), 0.23 (td, J = 9.5, 4.8 Hz, 1H), 0.04 (td, |
| | sulfanone | J = 9.7, 4.7 Hz, 1H); ESI MS (m/z): 422.1 [M + 1]. |
| 459 | ethyl(2-(pyrimidin-5- | ¹H-NMR (400 MHz, DMSO-d₆) δ 9.56 (s, 3H), 9.31 (s, 1H), |
| | yl)-2H-indazol-5- | 8.63 (d, J = 0.9 Hz, 1H), 8.46 (s, 1H), 8.39 (s, 1H), 7.96 (d, J = |
| | yl)((6- | 9.2 Hz, 1H), 7.73 (dd, J = 9.3, 1.7 Hz, 1H), 3.88-3.80 (m, 2H), |
| | (trifluoromethyl)pyrazin- | 1.25-1.16 (m, 3H); ESI MS (m/z): 434.1 [M + 1]. |
| | 2-yl)imino)-λ⁶- | |
| | sulfanone | |
| 463 | ethyl(2-(5- | ¹H-NMR (400 MHz, DMSO-d₆) δ 9.54 (d, J = 0.6 Hz, 1H), 9.27 |
| | fluoropyridin-3-yl)- | (s, 1H), 8.75 (d, J = 2.4 Hz, 1H), 8.60 (d, J = 0.9 Hz, 1H), 8.56 |
| | 2H-indazol-5-yl)((6- | (dt, J = 9.9, 2.3 Hz, 1H), 8.45 (s, 1H), 8.39 (s, 1H), 7.93 (d, J = |
| | (trifluoromethyl)pyrazin- | 9.2 Hz, 1H), 7.72 (dd, J = 9.3, 1.7 Hz, 1H), 3.88-3.78 (m, 2H), |
| | 2-yl)imino)-λ⁶- | 1.20 (t, J = 7.2 Hz, 3H); ESI MS (m/z): 451.1 [M + 1]. |
| | sulfanone | |
| 464 | ethyl(2-(5- | ¹H-NMR (400 MHz, DMSO-d₆) δ 9.52 (d, J = 0.6 Hz, 1H), 9.24 |
| | fluoropyridin-3-yl)- | (s, 1H), 8.75 (d, J = 2.4 Hz, 1H), 8.54-8.51 (m, 2H), 7.97 (t, J = |
| | 2H-indazol-5-yl)((2- | 5.5 Hz, 1H), 7.93 (s, 1H), 7.68-7.65 (m, 1H), 6.70 (d, J = 2.1 |
| | methylpyridin-4- | Hz, 1H), 6.60 (dd, J = 5.5, 2.1 Hz, 1H), 3.67-3.57 (m, 2H), 2.21 |
| | yl)imino)-λ⁶-sulfanone | (d, J = 14.4 Hz, 3H), 1.22-1.16 (m, 3H); ESI MS (m/z): 396.05 |
| | | [M + 1]. |
| 469 | (cyclobutylmethyl)(2- | ¹H-NMR (400 MHz, DMSO-d₆) δ 9.55 (s, 3H), 9.31 (s, 1H), |
| | (pyrimidin-5-yl)-2H- | 8.62 (d, J = 0.9 Hz, 1H), 8.44 (s, 1H), 8.39 (s, 1H), 7.95 (d, J = |
| | indazol-5-yl)((6- | 9.2 Hz, 1H), 7.74 (dd, J = 9.5, 1.8 Hz, 1H), 4.03-3.93 (m, 2H), |
| | (trifluoromethyl)pyrazin- | 2.71-2.65 (m, 1H), 1.99-1.93 (m, 1H), 1.85-1.57 (m, 5H) |
| | 2-yl)imino)-λ⁶- | ESI MS (m/z): 474 [M + 1]. |
| | sulfanone | |
| 473 | (cyclobutylmethyl)(2- | ¹H-NMR (400 MHz, CHLOROFORM-D) δ 9.05 (d, J = 1.8 Hz, |
| | (5-fluoropyridin-3-yl)- | 1H), 8.72 (d, J = 0.9 Hz, 1H), 8.61 (d, J = 2.4 Hz, 1H), 8.59 (q, |
| | 2H-indazol-5-yl)((6- | J = 0.8 Hz, 1H), 8.38 (s, 1H), 8.28 (s, 1H), 8.14 (dt, J = 8.9, 2.4 |
| | (trifluoromethyl)pyrazin- | Hz, 1H), 7.90-7.87 (m, 1H), 7.69 (dd, J = 9.3, 1.7 Hz, 1H), 3.82 |
| | 2-yl)imino)-λ⁶- | (q, J = 7.0 Hz, 1H), 3.71 (q, J = 7.2 Hz, 1H), 2.84 (t, J = 7.2 Hz, |
| | sulfanone | 1H), 2.14 (q, J = 4.2 Hz, 1H), 1.95-1.77 (m, 5H), 1.64 (t, J = 8.3 |
| | | Hz, 1H); ESI MS (m/z): 491.25 [M + 1]. |
| 476 | (cyclobutylmethyl)(2- | ¹H-NMR (400 MHz, DMSO-d₆) δ 9.51 (d, J = 0.7 Hz, 1H), 9.24 |
| | (5-fluoropyridin-3-yl)- | (s, 1H), 8.75 (d, J = 2.2 Hz, 1H), 8.56 (d, J = 1.0 Hz, 1H), 8.52 |
| | 2H-indazol-5-yl)((6- | (dt, J = 9.7, 2.3 Hz, 1H), 8.27 (d, J = 2.4 Hz, 1H), 7.95 (d, J = |
| | (trifluoromethyl)pyridin- | 9.3 Hz, 1H), 7.71 (dd, J = 9.2, 1.8 Hz, 1H), 7.55 (d, J = 8.6 Hz, |
| | 3-yl)imino)-λ⁶- | 1H), 7.34 (dd, J = 8.3, 2.2 Hz, 1H), 3.86 (d, J = 7.1 Hz, 2H), |
| | sulfanone | 2.71 (d, J = 7.6 Hz, 1H), 2.06-2.00 (m, 1H), 1.86-1.79 (m, 3H), |
| | | 1.69 (dd, J = 16.4, 7.6 Hz, 2H); ESI MS (m/z): 490.2 [M + 1]. |
| 477 | (cyclobutylmethyl)(2- | ¹H-NMR (400 MHz, DMSO-d₆) δ 9.53 (d, J = 0.7 Hz, 1H), 9.27 |
| | (5-fluoropyridin-3-yl)- | (s, 1H), 8.74 (d, J = 2.7 Hz, 1H), 8.57-8.53 (m, 2H), 7.91 (d, J = |
| | 2H-indazol-5-yl)((6- | 9.0 Hz, 1H), 7.78-7.70 (m, 2H), 7.17 (d, J = 7.3 Hz, 1H), 7.03 |
| | (trifluoromethyl)pyridin- | (d, J = 8.3 Hz, 1H), 3.92 (d, J = 7.1 Hz, 2H), 2.66-2.59 (m, 1H), |
| | 2-yl)imino)-λ⁶- | 1.98-1.93 (m, 1H), 1.80-1.70 (m, 3H), 1.66-1.55 (m, 2H) |
| | sulfanone | ESI MS (m/z): 490.05 [M + 1]. |
| 481 | (cyclobutylmethyl)((2- | ¹H-NMR (400 MHz, DMSO-d₆) δ 9.52 (s, 3H), 9.31 (s, 1H), |
| | methylpyridin-4- | 8.53 (s, 1H), 7.98-7.94 (m, 2H), 7.69-7.66 (m, 1H), 6.68 (d, J = |
| | yl)imino)(2- | 1.5 Hz, 1H), 6.58 (d, J = 5.5 Hz, 1H), 3.77 (d, J = 7.0 Hz, 2H), |
| | (pyrimidin-5-yl)-2H- | 2.73-2.65 (m, 1H), 2.23 (s, 3H), 1.98-1.95 (m, 1H), 1.85-1.80 |
| | indazol-5-yl)-λ⁶- | (m, 3H), 1.72-1.62 (m, 2H); ESI MS (m/z): 419.1 [M + 1]. |
| | sulfanone | |

### Example 10: Synthesis of N-((cyclobutylmethyl)(2-(5-fluoropyridin-3-yl)-2H-indazol-5-yl)(oxo)-λ⁶-sulfaneylidene) acetamide 472

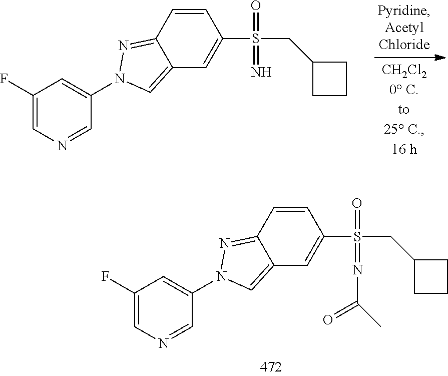 [SMILES: N=S(=O)(CC1CCC1)c1ccc2nn(-c3cncc(F)c3)cc2c1 ; CC(=O)N=S(=O)(CC1CCC1)c1ccc2nn(-c3cncc(F)c3)cc2c1]

To a stirred solution of (cyclobutylinethyl)(2-(5-fluoropyridin-3-yl)-2H-indazol-5-yl)(imino)-λ⁶-sulfanone (250 mg, 0.726 mmol) in dichloromethane (6 mL), pyridine (0.088 mL, 1.089 mmol) was added and the resulting reaction mixture was stirred for 10 minutes. The reaction mixture was cooled to 0° C. and acetyl chloride (0.057 mL, 0.798 mmol) was added into the reaction mixture which was stirred at 25° C. for 16 h. After completion of the reaction, the reaction mixture was poured into water (10 mL) and the aqueous layer was extracted with dichloromethane (3×20 mL). The organic layer was dried over anhydrous sodium sulphate, filtered and concentrated under reduced pressure to obtain a crude solid. The crude compound was purified by preparative HPLC to get the desired N-((cyclobutylmethyl)(2-(5-fluoropyridin-3-yl)-2H-indazol-5-yl)(oxo)-λ⁶-sulfaneylidene) acetamide 472 (110 mg, 0.285 mmol, 39.2 s yield).

TABLE 10
Representative compounds of the present disclosure were prepared according to
the suitable starting material and methods as described in the Example 10.

| Comp | | |
|---|---|---|
| no. | Compound name | Analytical Data |
| 389 | N-(isopropyl(oxo)(2- | ¹H-NMR (400 MHz, DMSO-D6) δ 9.49 (d, J = 0.9 Hz, 1H), |
| | (pyridin-3-yl)-2H- | 9.33 (d, J = 2.1 Hz, 1H), 8.71 (dd, J = 4.7, 1.4 Hz, 1H), 8.51 |
| | indazol-5-yl)-λ⁶- | (dq, J = 8.4, 1.3 Hz, 1H), 8.44 (t, J = 0.9 Hz, 1H), 7.97 (d, J = |
| | sulfaneylidene)acetamide | 9.2 Hz, 1H), 7.71-7.67 (m, 1H), 7.61 (dd, J = 9.2, 1.8 Hz, 1H), |
| | | 3.77-3.70 (m, 1H), 2.01 (s, 3H), 1.30 (d, J = 6.7 Hz, 3H), 1.25- |
| | | 1.22 (m, 1H), 1.12 (d, J = 6.7 Hz, 3H); ESI MS (m/z): 343.15 |
| | | [M + 1]. |
| 394 | 2,2,2-trifluoro-N- | ¹H-NMR (400 MHz, DMSO-d₆) δ 9.56 (d, J = 0.7 Hz, 1H), |
| | (isopropyl(oxo)(2- | 9.34 (d, J = 2.4 Hz, 1H), 8.73 (dd, J = 4.8, 1.3 Hz, 1H), 8.59 (d, |
| | (pyridin-3-yl)-2H- | J = 1.0 Hz, 1H), 8.52 (dq, J = 8.3, 1.4 Hz, 1H), 8.07 (d, J = 9.3 |
| | indazol-5-yl)-λ⁶- | Hz, 1H), 7.72-7.63 (m, 2H), 4.13-4.06 (m, 1H), 1.41 (d, J = 6.6 |
| | sulfaneylidene)acetamide | Hz, 3H), 1.22-1.18 (m, 3H); ESI MS (m/z): 396.5 [M + 1]. |
| 396 | 2,2-difluoro-N- | ¹H-NMR (400 MHz, DMSO-d₆) δ 9.54 (d, J = 0.9 Hz, 1H), |
| | (isopropyl(oxo)(2- | 9.34 (d, J = 2.4 Hz, 1H), 8.72 (dd, J = 4.6, 1.2 Hz, 1H), 8.54- |
| | (pyridin-3-yl)-2H- | 8.50 (m, 2H), 8.04 (d, J = 9.2 Hz, 1H), 7.71-7.68 (m, 1H), 7.64 |
| | indazol-5-yl)-λ⁶- | (dd, J = 9.2, 1.8 Hz, 1H), 6.22 (t, J = 54.3 Hz, 1H), 3.99-3.93 |
| | sulfaneylidene)acetamide | (m, 1H), 1.37 (d, J = 6.7 Hz, 3H), 1.17 (d, J = 6.7 Hz, 3H); ESI |
| | | MS (m/z): 378.45 [M + 1]. |
| 398 | 2,2,2-trifluoro-N-((2-(5- | ¹H-NMR (400 MHz, DMSO-d₆) δ 9.60 (s, 1H), 9.27 (s, 1H), |
| | fluoropyridin-3-yl)-2H- | 8.78 (d, J = 2.7 Hz, 1H), 8.61 (d, J = 1.7 Hz, 1H), 8.57 (dd, J = |
| | indazol-5- | 9.8, 2.2 Hz, 1H), 8.07 (d, J = 9.3 Hz, 1H), 7.66 (dd, J = 9.3, 2.0 |
| | yl)(isopropyl)(oxo)-λ⁶- | Hz, 1H), 4.10 (t, J = 6.8 Hz, 1H), 1.41 (d, J = 6.6 Hz, 3H), 1.22 |
| | sulfaneylidene)acetamide | (s, 1H), 1.18 (d, J = 6.6 Hz, 3H); ESI MS (m/z): 414.95 [M + 1]. |
| 399 | N-((2-(5-fluoropyridin-3- | ¹H-NMR (400 MHz, DMSO-D6) δ 9.53 (d, J = 0.7 Hz, 1H), |
| | yl)-2H-indazol-5- | 9.27 (s, 1H), 8.76 (d, J = 2.4 Hz, 1H), 8.56 (dt, J = 9.8, 2.3 Hz, |
| | yl)(isopropyl)(oxo)-λ⁶- | 1H), 8.45 (d, J = 1.0 Hz, 1H), 7.97 (d, J = 9.0 Hz, 1H), 7.62 |
| | sulfaneylidene)acetamide | (dd, J = 9.2, 1.8 Hz, 1H), 3.77-3.70 (m, 1H), 2.01 (s, 3H), 1.31 |
| | | (d, J = 6.8 Hz, 3H), 1.12 (d, J = 6.6 Hz, 3H); ESI MS (m/z): |
| | | 361.1 [M + 1]. |
| 401 | 2,2,2-trifluoro-N-((2-(5- | ¹H-NMR (400 MHz, DMSO-d₆) δ 9.63 (s, 1H), 9.31 (s, 1H), |
| | fluoropyridin-3-yl)-2H- | 9.16 (d, J = 2.4 Hz, 1H), 9.01 (d, J = 2.4 Hz, 1H), 8.82 (d, J = |
| | pyrazolo[3,4-b]pyridin-5- | 2.4 Hz, 1H), 8.64 (dt, J = 9.8, 2.3 Hz, 1H), 4.22 (m, J = 6.7 Hz, |
| | yl)(isopropyl)(oxo)-λ⁶- | 1H), 1.44 (d, J = 6.7 Hz, 3H), 1.24 (d, J = 6.7 Hz, 3H); ESI MS |
| | sulfaneylidene)acetamide | (m/z): 415.45 [M + 1]. |
| 402 | N-((2-(5-fluoropyridin-3- | ¹H-NMR (400 MHz, DMSO-D6) δ 9.55 (s, 1H), 9.30 (s, 1H), |
| | yl)-2H-pyrazolo[3,4- | 8.97 (d, J = 2.4 Hz, 1H), 8.93 (d, J = 2.4 Hz, 1H), 8.80 (d, J = |
| | b]pyridin-5- | 2.4 Hz, 1H), 8.62 (dt, J = 9.7, 2.3 Hz, 1H), 3.84 (q, J = 6.7 Hz, |
| | yl)(isopropyl)(oxo)-λ⁶- | 1H), 2.04 (s, 3H), 1.34 (d, J = 6.8 Hz, 3H), 1.18 (d, J = 6.6 Hz, |
| | sulfaneylidene)acetamide | 3H); ESI MS (m/z): 361.95 [M + 1]. |
| 403 | 2,2-difluoro-N-((2-(5- | ¹H-NMR (400 MHz, DMSO-d₆) δ 9.58 (s, 1H), 9.27 (s, 1H), |
| | fluoropyridin-3-yl)-2H- | 8.77 (d, J = 2.4 Hz, 1H), 8.59-8.56 (m, 2H), 8.03 (d, J = 9.3 Hz, |
| | indazol-5- | 1H), 7.65 (dd, J = 9.2, 1.8 Hz, 1H), 6.22 (t, J = 54.3 Hz, 1H), |
| | yl)(isopropyl)(oxo)-λ⁶- | 4.00-3.93 (m, 1H), 1.37 (d, J = 6.8 Hz, 3H), 1.17 (d, J = 6.6 Hz, |
| | sulfaneylidene)acetamide | 3H); ESI MS (m/z): 396.9 [M + 1]. |
| 404 | 2,2-difluoro-N-((2-(5- | ¹H-NMR (400 MHz, DMSO-d₆) δ 9.61 (s, 1H), 9.31 (s, 1H), |
| | fluoropyridin-3-yl)-2H- | 9.09 (d, J = 2.4 Hz, 1H), 8.99 (d, J = 2.4 Hz, 1H), 8.81 (d, J = |
| | pyrazolo[3,4-b]pyridin-5- | 2.4 Hz, 1H), 8.63 (dt, J = 9.8, 2.3 Hz, 1H), 6.25 (t, J = 54.2 Hz, |
| | yl)(isopropyl)(oxo)-λ⁶- | 1H), 4.08 (q, J = 6.8 Hz, 1H), 1.40 (d, J = 6.8 Hz, 3H), 1.23 (d, |
| | sulfaneylidene)acetamide | J = 6.6 Hz, 3H); ESI MS (m/z): 397.95 [M + 1]. |
| 413 | N- | ¹H-NMR (400 MHz, DMSO-d₆) δ 9.62 (d, J = 0.7 Hz, 1H), |
| | ((cyclopropylmethyl)(2- | 9.28 (d, J = 1.0 Hz, 1H), 8.78-8.77 (m, 1H), 8.66 (q, J = 0.8 Hz, |
| | (5-fluoropyridin-3-yl)- | 1H), 8.57 (dt, J = 9.8, 2.3 Hz, 1H), 8.08-8.05 (m, 1H), 7.75 (dd, |
| | 2H-indazol-5-yl)(oxo)- | J = 9.3, 2.0 Hz, 1H), 3.99 (ddd, J = 26.4, 14.7, 7.1 Hz, 2H), |
| | λ⁶-sulfaneylidene)-2,2,2- | 0.92-0.84 (m, 1H), 0.56-0.40 (m, 2H), 0.28 (td, J = 9.8, 4.6 Hz, |
| | trifluoroacetamide | 1H), 0.12 (td, J = 9.7, 4.5 Hz, 1H); ESI MS (m/z): 426.9 |
| | | [M + 1]. |
| 415 | N- | ¹H-NMR (400 MHz, DMSO-D6) δ 9.55 (s, 1H), 9.28 (s, 1H), |
| | ((cyclopropylmethyl)(2- | 8.76 (d, J = 2.4 Hz, 1H), 8.58-8.53 (m, 2H), 7.97 (d, J = 9.3 Hz, |
| | (5-fluoropyridin-3-yl)- | 1H), 7.72 (dd, J = 9.3, 1.7 Hz, 1H), 3.64 (d, J = 7.1 Hz, 2H), |
| | 2H-indazol-5-yl)(oxo)-λ⁶- | 2.01 (s, 3H), 0.85-0.81 (m, 1H), 0.45-0.36 (m, 2H), 0.15-0.02 |
| | sulfaneylidene)acetamide | (m, 2H); ESI MS (m/z): 372.95 [M + 1]. |
| 416 | N- | ¹H-NMR (400 MHz, DMSO-d₆) δ 9.60 (d, J = 1.0 Hz, 1H), |
| | ((cyclopropylmethyl)(2- | 9.28 (s, 1H), 8.77 (d, J = 2.7 Hz, 1H), 8.62 (t, J = 1.0 Hz, 1H), |
| | (5-fluoropyridin-3-yl)- | 8.57 (dt, J = 9.8, 2.3 Hz, 1H), 8.03 (d, J = 9.3 Hz, 1H), 7.74 |
| | 2H-indazol-5-yl)(oxo)- | (dd, J = 9.3, 2.0 Hz, 1H), 6.21 (t, J = 54.2 Hz, 1H), 3.91-3.81 |
| | λ⁶-sulfaneylidene)-2,2- | (m, 2H), 0.91-0.84 (m, 1H), 0.52-0.38 (m, 2H), 0.22 (td, J = |
| | difluoroacetamide | 9.5, 4.8 Hz, 1H), 0.09 (td, J = 9.6, 4.6 Hz, 1H); ESI MS (m/z): |
| | | 408.95 [M + 1]. |
| 417 | 2,2,2-trifluoro-N- | ¹H-NMR (400 MHz, DMSO-d₆) δ 9.62 (d, J = 1.0 Hz, 1H), |
| | (isopropyl(oxo)(2- | 9.56 (s, 2H), 9.34 (s, 1H), 8.64 (t, J = 1.0 Hz, 1H), 8.09 (d, J = |
| | (pyrimidin-5-yl)-2H- | 9.3 Hz, 1H), 7.67 (dd, J = 9.3, 2.0 Hz, 1H), 4.14-4.07 (m, 1H), |
| | indazol-5-yl)-λ⁶- | 1.41 (d, J = 6.6 Hz, 3H), 1.18 (d, J = 6.6 Hz, 3H); ESI MS |
| | sulfaneylidene)acetamide | (m/z): 398.0 [M + 1]. |
| 418 | N-(isopropyl(oxo)(2- | ¹H-NMR (400 MHz, DMSO-D6) δ 9.56-9.55 (m, 3H), 9.33 (s, |
| | (pyrimidin-5-yl)-2H- | 1H), 8.48 (t, J = 0.9 Hz, 1H), 7.99 (d, J = 9.2 Hz, 1H), 7.63 (dd, |
| | indazol-5-yl)-λ⁶- | J = 9.3, 1.7 Hz, 1H), 3.77-3.71 (m, 1H), 2.00 (d, J = 4.9 Hz, |
| | sulfaneylidene)acetamide | 3H), 1.30 (d, J = 6.7 Hz, 3H), 1.12 (d, J = 6.7 Hz, 3H); ESI MS |
| | | (m/z): 343.5 [M + 1]. |
| 421 | 2,2-difluoro-N- | ¹H-NMR (400 MHz, DMSO-d₆) δ 9.60 (d, J = 0.9 Hz, 1H), |
| | (isopropyl(oxo)(2- | 9.56 (s, 2H), 9.33 (s, 1H), 8.59 (q, J = 0.8 Hz, 1H), 8.05 (d, J = |
| | (pyrimidin-5-yl)-2H- | 9.2 Hz, 1H), 7.66 (dd, J = 9.5, 1.8 Hz, 1H), 6.22 (t, J = 54.3 Hz, |
| | indazol-5-yl)-λ⁶- | 1H), 4.00-3.93 (m, 1H), 1.37 (d, J = 6.4 Hz, 3H), 1.17 (d, J = |
| | sulfaneylidene)acetamide | 6.7 Hz, 3H); ESI MS (m/z): 379.9 [M + 1]. |
| 422 | 2,2,3,3,3-pentafluoro-N- | ¹H-NMR (400 MHz, DMSO-d₆) δ 9.63 (d, J = 0.9 Hz, 1H), |
| | (isopropyl(oxo)(2- | 9.56 (s, 2H), 9.34 (s, 1H), 8.62 (t, J = 0.9 Hz, 1H), 8.10 (d, J = |
| | (pyrimidin-5-yl)-2H- | 9.2 Hz, 1H), 7.63 (dd, J = 9.2, 1.8 Hz, 1H), 4.11 (t, J = 6.7 Hz, |
| | indazol-5-yl)-λ⁶-sul- | 1H), 1.41 (d, J = 6.7 Hz, 3H), 1.19 (d, J = 6.7 Hz, 3H); ESI MS |
| | faneylidene)propanamide | (m/z): 447.9 [M + 1]. |
| 456 | N-(ethyl(oxo)(2- | ¹H-NMR (400 MHz, DMSO-D6) δ 9.56 (s, 3H), 9.32 (s, 1H), |
| | (pyrimidin-5-yl)-2H- | 8.53 (t, J = 0.9 Hz, 1H), 8.00 (d, J = 9.2 Hz, 1H), 7.70 (dd, J = |
| | indazol-5-yl)-λ⁶-sul- | 9.3, 1.7 Hz, 1H), 3.67-3.57 (m, 2H), 2.01 (s, 3H), 1.10 (t, J = |
| | faneylidene)acetamide | 7.3 Hz, 3H); ESI MS (m/z): 330 [M + 1]. |
| 462 | N-(ethyl(2-(5- | ¹H-NMR (400 MHz, DMSO-D6) δ 9.54 (d, J = 1.0 Hz, 1H), |
| | fluoropyridin-3-yl)-2H- | 9.27 (s, 1H), 8.76 (d, J = 2.7 Hz, 1H), 8.56 (dt, J = 9.9, 2.3 Hz, |
| | indazol-5-yl)(oxo)-λ⁶- | 1H), 8.50 (d, J = 1.0 Hz, 1H), 7.98 (d, J = 9.3 Hz, 1H), 7.69 |
| | sulfaneylidene)acetamide | (dd, J = 9.3, 1.7 Hz, 1H), 3.65-3.58 (m, 2H), 2.00 (s, 3H), 1.10 |
| | | (t, J = 7.2 Hz, 3H); ESI MS (m/z): 346.9 [M + 1]. |
| 467 | N- | ¹H-NMR (400 MHz, DMSO-D6) δ 9.56 (t, J = 1.2 Hz, 3H), |
| | ((cyclobutylmethyl)(oxo) | 9.32 (s, 1H), 8.52 (q, J = 0.8 Hz, 1H), 7.99 (d, J = 9.2 Hz, 1H), |
| | (2-(pyrimidin-5-yl)-2H- | 7.70 (dd, J = 9.5, 1.8 Hz, 1H), 3.78 (d, J = 7.0 Hz, 2H), 2.58- |
| | indazol-5-yl)-λ⁶- | 2.54 (m, 1H), 1.99 (s, 3H), 1.93-1.89 (m, 1H), 1.79-1.57 (m, |
| | sulfaneylidene)acetamide | 5H); ESI MS (m/z): 369.95 [M + 1]. |
| 472 | N-((cyclobutylmethyl)(2- | ¹H-NMR (400 MHz, CHLOROFORM-D) δ 9.05 (s, 1H), 8.72 |
| | (5-fluoropyridin-3-yl)- | (s, 1H), 8.61 (d, J = 1.8 Hz, 1H), 8.53 (t, J = 0.8 Hz, 1H), 8.14 |
| | 2H-indazol-5-yl)(oxo)- | (dt, J = 8.9, 2.3 Hz, 1H), 7.92 (d, J = 9.2 Hz, 1H), 7.69 (dd, J = |
| | λ⁶- | 9.2, 1.8 Hz, 1H), 3.73 (dd, J = 14.2, 6.6 Hz, 1H), 3.59 (q, J = 7.3 |
| | sulfaneylidene)acetamide | Hz, 1H), 2.71 (q, J = 7.6 Hz, 1H), 2.17 (s, 3H), 2.09-2.05 (m, |
| | | 1H), 1.91-1.74 (m, 4H), 1.64-1.60 (m, 1H); ESI MS (m/z): 387.1 |
| | | [M + 1]. |
| 475 | N-(methyl(oxo)(2- | ¹H-NMR (400 MHz, CHLOROFORM-D) δ 9.38 (s, 2H), 9.33 |
| | (pyrimidin-5-yl)-2H- | (s, 1H), 8.74 (d, J = 0.9 Hz, 1H), 8.60 (q, J = 0.8 Hz, 1H), 7.97 |
| | indazol-5-yl)-λ⁶- | (dd, J = 8.6, 0.9 Hz, 1H), 7.75 (dd, J = 9.2, 1.8 Hz, 1H), 3.40 (s, |
| | sulfaneylidene)acetamide | 3H), 2.19 (s, 3H); ESI MS (m/z): 316.05 [M + 1]. |
| 483 | N-((2-(5-fluoropyridin-3- | ¹H-NMR (400 MHz, DMSO-D6) δ 9.54 (d, J = 1.0 Hz, 1H), |
| | yl)-2H-indazol-5- | 9.27 (s, 1H), 8.76 (d, J = 2.4 Hz, 1H), 8.56 (dt, J = 9.8, 2.3 Hz, |
| | yl)(oxo)(propyl)-λ⁶- | 1H), 8.50 (t, J = 0.9 Hz, 1H), 7.98 (d, J = 9.0 Hz, 1H), 7.69 (dd, |
| | sulfaneylidene)acetamide | J = 9.3, 1.7 Hz, 1H), 3.66-3.52 (m, 2H), 2.00 (s, 3H), 1.63-1.45 |
| | | (m, 2H), 0.89 (t, J = 7.3 Hz, 3H); ESI MS (m/z): 361 [M + 1]. |
| 485 | N-((2-(5-fluoropyridin-3- | ¹H-NMR (400 MHz, DMSO-D6) δ 9.52 (s, 1H), 9.27 (s, 1H), |
| | yl)-2H-indazol-5- | 8.76 (d, J = 2.4 Hz, 1H), 8.56 (dd, J = 7.5, 2.3 Hz, 1H), 8.47 (s, |
| | yl)(oxo)(pentan-3-yl)-λ⁶- | 1H), 7.97 (d, J = 9.3 Hz, 1H), 7.64 (dd, J = 9.3, 1.7 Hz, 1H), |
| | sulfaneylidene)acetamide | 3.39 (d, J = 6.6 Hz, 1H), 2.01-1.96 (m, 4H), 1.76 (td, J = 14.7, |
| | | 7.7 Hz, 2H), 1.49 (t, J = 7.2 Hz, 1H), 0.99 (t, J = 7.5 Hz, 3H), |
| | | 0.85 (t, J = 7.5 Hz, 3H); ESI MS (m/z): 389.05 [M + 1]. |
| 486 | N-(sec-butyl(2-(5- | ¹H-NMR (400 MHz, DMSO-D6) δ 9.45 (dd, J = 13.0, 5.0 Hz, |
| | fluoropyridin-3-yl)-2H- | 1H), 9.24 (d, J = 2.1 Hz, 1H), 8.72 (q, J = 2.4 Hz, 1H), 8.50- |
| | indazol-5-yl)(oxo)-λ⁶- | 8.45 (m, 2H), 7.95 (q, J = 4.6 Hz, 1H), 7.65-7.62 (m, 1H), 3.58- |
| | sulfaneylidene)acetamide | 3.54 (m, 1H), 2.18-1.80 (m, 4H), 1.48-1.15 (m, 4H), 0.94 (dtd, |
| | | J = 32.3, 7.4, 4.1 Hz, 3H); ESI MS (m/z): 375.05 [M + 1]. |
| 487 | N-((3-fluoropropyl)(2-(5- | ¹H-NMR (400 MHz, DMSO-D6) δ 9.55 (d, J = 0.7 Hz, 1H), |
| | fluoropyridin-3-yl)-2H- | 9.28 (s, 1H), 8.76 (d, J = 2.4 Hz, 1H), 8.58-8.54 (m, 2H), 7.99 |
| | indazol-5-yl)(oxo)-λ⁶- | (d, J = 9.3 Hz, 1H), 7.71 (dd, J = 9.3, 2.0 Hz, 1H), 4.54-4.39 |
| | sulfaneylidene)acetamide | (m, 2H), 3.80-3.63 (m, 2H), 2.06-1.86 (m, 5H) |
| | | ESI MS (m/z): 379.1 [M + 1]. |
| 565 | N-(cyclobutyl(oxo)(2- | ¹H-NMR (400 MHz, DMSO-d₆) δ 9.48 (d, J = 0.6 Hz, 1H), 9.34 |
| | (pyridin-3-yl)-2H- | (q, J = 2.5 Hz, 1H), 8.71 (dd, J = 4.7, 1.4 Hz, 1H), 8.53-8.49 (m, |
| | indazol-5-yl)-λ⁶- | 1H), 8.45 (q, J = 0.8 Hz, 1H), 7.98-7.94 (m, 1H), 7.72-7.67 (m, |
| | sulfaneylidene)acetamide | 1H), 7.60 (dd, J = 9.3, 1.7 Hz, 1H), 4.37 (t, J = 7.9 Hz, 1H), 2.56- |
| | | 2.53 (m, 1H), 2.32-2.20 (m, 2H), 1.97 (d, J = 7.3 Hz, 3H), 1.93- |
| | | 1.81 (m, 3H); ESI MS (m/z): 354.9 [M + 1]. |
| 566 | N- | ¹H-NMR (400 MHz, DMSO-d₆) δ 9.50 (d, J = 0.9 Hz, 1H), 9.34 |
| | ((cyclohexylmethyl)(oxo) | (d, J = 2.4 Hz, 1H), 8.71 (dd, J = 4.7, 1.4 Hz, 1H), 8.53-8.50 (m, |
| | (2-(pyridin-3-yl)-2H- | 2H), 7.97 (d, J = 9.2 Hz, 1H), 7.72-7.67 (m, 2H), 3.54 (dd, J = |
| | indazol-5-yl)-λ⁶- | 19.6, 5.8 Hz, 2H), 1.97 (s, 3H), 1.79 (s, 2H), 1.61-1.51 (m, 4H), |
| | sulfaneylidene)acetamide | 1.08 (dd, J = 15.7, 8.4 Hz, 5H); ESI MS (m/z): 396.95 [M + 1]. |
| 570 | N-(ethyl(2-(5- | ¹H-NMR (400 MHz, DMSO-d₆) δ 9.55 (d, J = 1.0 Hz, 1H), 9.27 |
| | fluoropyridin-3-yl)-2H- | (s, 1H), 8.76 (d, J = 2.2 Hz, 1H), 8.56 (dt, J = 9.9, 2.3 Hz, 1H), |
| | indazol-5-yl)(oxo)-λ⁶-sul- | 8.50 (q, J = 0.8 Hz, 1H), 7.99-7.96 (m, 1H), 7.68 (dd, J = 9.3, 2.0 |
| | faneylidene)propionamide | Hz, 1H), 3.69-3.58 (m, 2H), 2.30 (q, J = 7.5 Hz, 2H), 1.10 (t, J = |
| | | 7.3 Hz, 3H), 0.99 (t, J = 7.6 Hz, 3H); ESI MS (m/z): 361 [M + 1]. |
| 571 | N-(ethyl(2-(5- | ¹H-NMR (400 MHz, DMSO-d₆) δ 9.56 (d, J = 1.0 Hz, 1H), 9.27 |
| | fluoropyridin-3-yl)-2H- | (s, 1H), 8.76 (d, J = 2.4 Hz, 1H), 8.56 (dt, J = 9.9, 2.4 Hz, 1H), |
| | indazol-5-yl)(oxo)-λ⁶-sul- | 8.49 (q, J = 0.9 Hz, 1H), 7.98 (d, J = 9.3 Hz, 1H), 7.67 (dd, J = |
| | faneylidene)isobutyramide | 9.3, 1.7 Hz, 1H), 3.63 (q, J = 7.3 Hz, 2H), 2.46 (s, 1H), 1.09 (q, |
| | | J = 7.0 Hz, 9H); ESI MS (m/z): 375.05 [M + 1]. |
| 572 | N- | ¹H-NMR (400 MHz, DMSO-d₆) δ 9.56 (d, J = 2.4 Hz, 3H), 9.32 |
| | ((cyclobutylmethyl)(oxo) | (s, 1H), 8.50 (d, J = 0.9 Hz, 1H), 7.99 (d, J = 9.5 Hz, 1H), 7.68 |
| | (2-(pyrimidin-5-yl)-2H- | (dd, J = 9.2, 1.8 Hz, 1H), 3.79 (d, J = 7.3 Hz, 2H), 2.56 (d, J = |
| | indazol-5-yl)-λ⁶- | 6.4 Hz, 1H), 1.93-1.90 (m, 1H), 1.79-1.73 (m, 3H), 1.69-1.59 |
| | sulfaneylidene)cyclopro- | (m, 3H), 0.80-0.73 (m, 4H); ESI MS (m/z): 396 [M + 1]. |
| | panecarboxamide | |
| 573 | N- | ¹H-NMR (400 MHz, DMSO-d₆) δ 9.56 (s, 3H), 9.32 (s, 1H), 8.52 |
| | ((cyclobutylmethyl)(oxo) | (d, J = 0.9 Hz, 1H), 7.99 (d, J = 9.5 Hz, 1H), 7.70 (dd, J = 9.2, |
| | (2-(pyrimidin-5-yl)-2H- | 1.8 Hz, 1H), 3.78 (d, J = 7.3 Hz, 2H), 2.57-2.53 (m, 1H), 2.29 |
| | indazol-5-yl)-λ⁶-sul- | (q, J = 7.5 Hz, 2H), 1.94-1.89 (m, 1H), 1.79-1.58 (m, 5H), 0.99 |
| | faneylidene)propionamide | (t, J = 7.5 Hz, 3H); ESI MS (m/z): 384 [M + 1]. |
| 574 | N-((cyclobutylmethyl)(2- | ¹H-NMR (400 MHz, DMSO-d₆) δ 9.55 (d, J = 1.0 Hz, 1H), 9.27 |
| | (5-fluoropyridin-3-yl)- | (s, 1H), 8.76 (d, J = 2.2 Hz, 1H), 8.56 (dt, J = 9.7, 2.3 Hz, 1H), |
| | 2H-indazol-5-yl)(oxo)- | 8.47 (q, J = 0.9 Hz, 1H), 7.97 (d, J = 9.3 Hz, 1H), 7.67 (dd, J = |
| | λ⁶- | 9.3, 1.7 Hz, 1H), 3.83-3.74 (m, 2H), 2.59-2.51 (m, 1H), 1.96- |
| | sulfaneylidene)cyclopro- | 1.87 (m, 1H), 1.80-1.57 (m, 6H), 0.84-0.73 (m, 4H) |
| | panecarboxamide | ESI MS (m/z): 413.05 [M + 1]. |
| 575 | N-((cyclobutylmethyl)(2- | ¹H-NMR (400 MHz, DMSO-d₆) δ 9.54 (d, J = 1.0 Hz, 1H), 9.27 |
| | (5-fluoropyridin-3-yl)- | (t, J = 1.1 Hz, 1H), 8.75 (d, J = 2.2 Hz, 1H), 8.56 (dt, J = 9.8, 2.3 |
| | 2H-indazol-5-yl)(oxo)- | Hz, 1H), 8.48 (q, J = 0.8 Hz, 1H), 7.97 (d, J = 9.3 Hz, 1H), 7.68 |
| | λ⁶- | (dd, J = 9.3, 2.0 Hz, 1H), 3.82-3.73 (m, 2H), 2.58-2.51 (m, 1H), |
| | sulfaneylidene)butyramide | 2.27-2.20 (m, 2H), 1.95-1.87 (m, 1H), 1.81-1.60 (m, 5H), 1.58- |
| | | 1.48 (m, 2H), 0.87 (t, J = 7.5 Hz, 3H); ESI MS (m/z): 415.05 |
| | | [M + 1]. |
| 576 | N-((cyclobutylmethyl)(2- | ¹H-NMR (400 MHz, DMSO-d₆) δ 9.55 (d, J = 1.0 Hz, 1H), 9.27 |
| | (5-fluoropyridin-3-yl)- | (s, 1H), 8.76 (d, J = 2.2 Hz, 1H), 8.56 (dt, J = 9.7, 2.3 Hz, 1H), |
| | 2H-indazol-5-yl)(oxo)- | 8.48 (t, J = 0.9 Hz, 1H), 7.97 (d, J = 9.0 Hz, 1H), 7.67 (dd, J = |
| | λ⁶-sul- | 9.2, 1.8 Hz, 1H), 3.78 (d, J = 7.3 Hz, 2H), 2.54 (d, J = 8.1 Hz, |
| | faneylidene)isobutyramide | 1H), 2.46 (d, J = 6.8 Hz, 1H), 1.95-1.90 (m, 1H), 1.78-1.60 (m, |
| | | 5H), 1.06 (d, J = 7.3 Hz, 6H); ESI MS (m/z): 415.1 [M + 1]. |
| 577 | N-(ethyl(2-(5- | ¹H-NMR (400 MHz, DMSO-d₆) δ 9.55 (d, J = 1.0 Hz, 1H), 9.27 |
| | fluoropyridin-3-yl)-2H- | (d, J = 1.0 Hz, 1H), 8.76 (d, J = 2.2 Hz, 1H), 8.56 (dt, J = 9.8, |
| | indazol-5-yl)(oxo)-λ⁶- | 2.3 Hz, 1H), 8.48 (q, J = 0.8 Hz, 1H), 7.98 (dt, J = 9.2, 0.9 Hz, |
| | sulfaneylidene)cyclopro- | 1H), 7.67 (dd, J = 9.3, 2.0 Hz, 1H), 3.68-3.56 (m, 2H), 1.68-1.62 |
| | panecarboxamide | (m, 1H), 1.10 (t, J = 7.3 Hz, 3H), 0.82-0.71 (m, 4H); ESI MS |
| | | (m/z): 373 [M + 1]. |
| 578 | N-(ethyl(2-(5- | ¹H-NMR (400 MHz, DMSO-d₆) δ 9.55 (d, J = 1.0 Hz, 1H), 9.27 |
| | fluoropyridin-3-yl)-2H- | (d, J = 1.2 Hz, 1H), 8.75 (d, J = 2.2 Hz, 1H), 8.55 (dt, J = 9.9, |
| | indazol-5-yl)(oxo)-λ⁶- | 2.4 Hz, 1H), 8.49 (q, J = 0.9 Hz, 1H), 7.99-7.96 (m, 1H), 7.67 |
| | sulfaneylidene)butyramide | (dd, J = 9.3, 1.7 Hz, 1H), 3.69-3.56 (m, 2H), 2.29-2.22 (m, 2H), |
| | | 1.53 (td, J = 14.7, 7.3 Hz, 2H), 1.11-1.04 (m, 3H), 0.93-0.85 (m, |
| | | 3H); ESI MS (m/z): 375 [M + 1]. |
| 579 | N-((cyclobutylmethyl)(2- | ¹H-NMR (400 MHz, DMSO-d₆) δ 9.54 (d, J = 0.5 Hz, 1H), 9.27 |
| | (5-fluoropyridin-3-yl)- | (s, 1H), 8.75 (d, J = 2.4 Hz, 1H), 8.55 (dt, J = 9.8, 2.3 Hz, 1H), |
| | 2H-indazol-5-yl)(oxo)- | 8.49 (d, J = 1.0 Hz, 1H), 7.96 (d, J = 9.3 Hz, 1H), 7.68 (dd, J = |
| | λ⁶-sul- | 9.2, 1.8 Hz, 1H), 3.82-3.73 (m, 2H), 2.57-2.52 (m, 1H), 2.28 (q, |
| | faneylidene)propionamide | J = 7.5 Hz, 2H), 1.95-1.87 (m, 1H), 1.79-1.58 (m, 5H), 0.99 (t, |
| | | J = 7.5 Hz, 3H); ESI MS (m/z): 401 [M + 1]. |
| 581 | N-(cyclopentyl(oxo)(2- | ¹H-NMR (400 MHz, DMSO-d₆) δ 9.48 (s, 1H), 9.33 (d, J = 2.4 |
| | (pyridin-3-yl)-2H- | Hz, 1H), 8.71 (dd, J = 4.8, 1.3 Hz, 1H), 8.52-8.49 (m, 1H), 8.47 |
| | indazol-5-yl)-λ⁶- | (s, 1H), 7.96 (d, J = 9.0 Hz, 1H), 7.69 (dd, J = 8.8, 4.9 Hz, 1H), |
| | sulfaneylidene)acetamide | 7.64 (dd, J = 9.3, 1.7 Hz, 1H), 4.04 (t, J = 7.9 Hz, 1H), 2.13-2.04 |
| | | (m, 2H), 1.97 (s, 3H), 1.75 (dd, J = 13.0, 7.6 Hz, 1H), 1.66-1.50 |
| | | (m, 5H); ESI MS (m/z): 369 [M + 1]. |
| 582 | N-(isopentyl(oxo)(2- | ¹H-NMR (400 MHz, DMSO-d₆) δ 9.50 (s, 1H), 9.34 (d, J = 2.4 |
| | (pyridin-3-yl)-2H- | Hz, 1H), 8.71 (dd, J = 4.6, 1.2 Hz, 1H), 8.53-8.50 (m, 2H), 7.98 |
| | indazol-5-yl)-λ⁶- | (d, J = 9.3 Hz, 1H), 7.72-7.67 (m, 2H), 3.68-3.55 (m, 2H), 2.00 |
| | sulfaneylidene)acetamide | (s, 3H), 1.57 (td, J = 13.3, 6.7 Hz, 1H), 1.52-1.43 (m, 1H), 1.36- |
| | | 1.27 (m, 1H), 0.80 (dd, J = 6.5, 4.3 Hz, 6H); ESI MS (m/z): 371 |
| | | [M + 1]. |
| 583 | N-((3- | ¹H-NMR (400 MHz, DMSO-d₆) δ 9.51 (s, 1H), 9.34 (d, J = 2.4 |
| | cyanopropyl)(oxo)(2- | Hz, 1H), 8.72 (dd, J = 4.8, 1.3 Hz, 1H), 8.53-8.50 (m, 2H), 8.00 |
| | (pyridin-3-yl)-2H- | (d, J = 9.3 Hz, 1H), 7.71-7.68 (m, 2H), 3.77-3.60 (m, 2H), 2.61 |
| | indazol-5-yl)-λ⁶- | (t, J = 7.3 Hz, 2H), 2.03 (s, 3H), 1.94-1.79 (m, 2H); ESI MS |
| | sulfaneylidene)acetamide | (m/z): 368 [M + 1]. |
| 584 | N-((3- | ¹H-NMR (400 MHz, DMSO-d₆) δ 9.52 (s, 1H), 9.34 (d, J = 2.4 |
| | cyanopropyl)(oxo)(2- | Hz, 1H), 8.72-8.71 (m, 1H), 8.51 (d, J = 7.3 Hz, 2H), 8.00 (d, |
| | (pyridin-3-yl)-2H- | J = 9.3 Hz, 1H), 7.71-7.65 (m, 2H), 3.79-3.61 (m, 2H), 2.61 (t, |
| | indazol-5-yl)-λ⁶- | J = 7.2 Hz, 2H), 1.93-1.78 (m, 2H), 1.65 (dd, J = 13.0, 6.8 Hz, |
| | sulfaneylidene)cyclopro- | 1H), 0.83-0.77 (m, 4H); ESI MS (m/z): 394 [M + 1]. |
| | panecarboxamide | |
| 590 | N-(oxo(pentan-3-yl)(2- | ¹H-NMR (400 MHz, DMSO-d₆) δ 9.48 (d, J = 0.7 Hz, 1H), 9.33 |
| | (pyridin-3-yl)-2H- | (d, J = 2.7 Hz, 1H), 8.71 (dd, J = 4.8, 1.3 Hz, 1H), 8.51 (dq, J = |
| | indazol-5-yl)-λ⁶- | 8.3, 1.4 Hz, 1H), 8.46 (d, J = 1.0 Hz, 1H), 7.97 (d, J = 9.0 Hz, |
| | sulfaneylidene)acetamide | 1H), 7.69 (dd, J = 8.3, 4.9 Hz, 1H), 7.63 (dd, J = 9.3, 2.0 Hz, |
| | | 1H), 3.38 (t, J = 7.1 Hz, 1H), 2.03-1.96 (m, 4H), 1.80-1.68 (m, |
| | | 2H), 1.53-1.46 (m, 1H), 0.99 (t, J = 7.6 Hz, 3H), 0.85 (t, J = 7.6 |
| | | Hz, 3H); ESI MS (m/z): 370.95 [M + 1]. |
| 591 | N-(isobutyl(oxo)(2- | ¹H-NMR (400 MHz, DMSO-d₆) δ 9.49 (d, J = 0.7 Hz, 1H), 9.34 |
| | (pyridin-3-yl)-2H- | (d, J = 2.4 Hz, 1H), 8.71 (dd, J = 4.6, 1.5 Hz, 1H), 8.52-8.49 (m, |
| | indazol-5-yl)-λ⁶- | 2H), 7.98 (d, J = 9.3 Hz, 1H), 7.72-7.67 (m, 2H), 3.59 (dd, J = |
| | sulfaneylidene)acetamide | 14.4, 6.4 Hz, 1H), 3.49 (dd, J = 14.4, 6.4 Hz, 1H), 2.02 (t, J = 6.6 |
| | | Hz, 1H), 1.98 (s, 3H), 0.98 (d, J = 6.8 Hz, 3H), 0.88 (d, J = 6.8 |
| | | Hz, 3H); ESI MS (m/z): 356.9 [M + 1]. |
| 592 | N-(cycloheptyl(oxo)(2- | ¹H-NMR (400 MHz, DMSO-d₆) δ 9.48 (d, J = 0.6 Hz, 1H), 9.33 |
| | (pyridin-3-yl)-2H- | (d, J = 2.4 Hz, 1H), 8.71 (dd, J = 4.7, 1.4 Hz, 1H), 8.51 (dq, J = |
| | indazol-5-yl)-λ⁶- | 8.3, 1.4 Hz, 1H), 8.45 (d, J = 0.9 Hz, 1H), 7.96 (d, J = 9.2 Hz, |
| | sulfaneylidene)acetamide | 1H), 7.69 (q, J = 4.2 Hz, 1H), 7.63 (dd, J = 9.2, 1.8 Hz, 1H), |
| | | 3.69-3.64 (m, 1H), 2.30-2.27 (m, 1H), 2.00 (s, 3H), 1.95-1.90 |
| | | (m, 1H), 1.68-1.60 (m, 3H), 1.53-1.34 (m, 7H); ESI MS (m/z): |
| | | 397.05 [M + 1]. |
| 593 | N-((2- | ¹H-NMR (400 MHz, DMSO-d₆) δ 9.50 (d, J = 0.9 Hz, 1H), 9.34 |
| | cyclohexylethyl)(oxo)(2- | (d, J = 2.4 Hz, 1H), 8.71 (dd, J = 4.7, 1.4 Hz, 1H), 8.53-8.50 (m, |
| | (pyridin-3-yl)-2H- | 2H), 7.97 (d, J = 9.2 Hz, 1H), 7.71-7.67 (m, 2H), 3.67-3.60 (m, |
| | indazol-5-yl)-λ⁶- | 2H), 1.99 (s, 3H), 1.59-1.51 (m, 5H), 1.50-1.44 (m, 1H), 1.34- |
| | sulfaneylidene)acetamide | 1.21 (m, 2H), 1.11-1.01 (m, 3H), 0.78 (dd, J = 21.5, 9.9 Hz, 2H) |
| | | ESI MS (m/z): 411.1 [M + 1]. |
| 594 | N-(oxo(pentan-2-yl)(2- | ¹H-NMR (400 MHz, DMSO-d₆) δ 9.48 (s, 1H), 9.33 (d, J = 2.7 |
| | (pyridin-3-yl)-2H- | Hz, 1H), 8.71 (dd, J = 4.6, 1.5 Hz, 1H), 8.51 (d, J = 9.3 Hz, 1H), |
| | indazol-5-yl)-λ⁶- | 8.44 (s, 1H), 7.97 (d, J = 8.6 Hz, 1H), 7.69 (dd, J = 8.2, 4.8 Hz, |
| | sulfaneylidene)acetamide | 1H), 7.61 (dd, J = 9.3, 1.2 Hz, 1H), 3.62-3.58 (m, 1H), 2.00 (d, |
| | | J = 6.6 Hz, 4H), 1.35-1.11 (m, 6H), 0.83 (dt, J = 36.4, 7.2 Hz, |
| | | 3H); ESI MS (m/z): 370.85 [M + 1]. |
| 596 | N-(oxo(2-(pyridin-3-yl)- | ¹H-NMR (400 MHz, DMSO-d₆) δ 9.50 (d, J = 4.2 Hz, 1H), 9.34 |
| | 2H-indazol-5- | (d, J = 2.7 Hz, 1H), 8.71 (d, J = 4.6 Hz, 1H), 8.51 (d, J = 10.8 |
| | yl)((tetrahydrofuran-2- | Hz, 2H), 7.95 (dd, J = 9.3, 4.2 Hz, 1H), 7.74-7.67 (m, 2H), 4.16- |
| | yl)methyl)-λ⁶- | 4.05 (m, 1H), 3.93-3.82 (m, 2H), 3.59-3.42 (m, 2H), 2.00 (d, J = |
| | sulfaneylidene)acetamide | 16.6 Hz, 3H), 1.96-1.84 (m, 1H), 1.80-1.66 (m, 2H), 1.63-1.50 |
| | | (m, 1H); ESI MS (m/z): 384.9 [M + 1]. |
| 598 | N- | ¹H-NMR (400 MHz, DMSO-d₆) δ 9.50 (s, 1H), 9.34 (d, J = 2.4 |
| | ((cyclohexylmethyl)(oxo) | Hz, 1H), 8.71 (d, J = 3.4 Hz, 1H), 8.51 (d, J = 9.5 Hz, 2H), 7.97 |
| | (2-(pyridin-3-yl)-2H- | (d, J = 9.3 Hz, 1H), 7.70-7.67 (m, 2H), 3.58 (d, J = 5.9 Hz, 1H), |
| | indazol-5-yl)-λ⁶- | 3.52 (d, J = 5.9 Hz, 1H), 1.79 (s, 2H), 1.63-1.51 (m, 5H), 1.11- |
| | sulfaneylidene)cyclopro- | 1.00 (m, 5H), 0.76-0.73 (m, 4H); ESI MS (m/z): 423.15 [M + 1]. |
| | panecarboxamide | |
| 599 | N- | ¹H-NMR (400 MHz, DMSO-d₆) δ 9.50 (s, 1H), 9.34 (d, J = 2.4 |
| | ((cyclohexylmethyl)(oxo) | Hz, 1H), 8.71 (d, J = 3.4 Hz, 1H), 8.52 (s, 2H), 7.97 (d, J = 9.3 |
| | (2-(pyridin-3-yl)-2H- | Hz, 1H), 7.72-7.67 (m, 2H), 3.57 (d, J = 5.9 Hz, 1H), 3.52 (d, |
| | indazol-5-yl)-λ⁶-sul- | J = 5.9 Hz, 1H), 2.27 (q, J = 7.5 Hz, 2H), 1.79 (s, 2H), 1.61-1.51 |
| | faneylidene)propionamide | (m, 4H), 1.11-1.05 (m, 4H), 0.97 (t, J = 7.6 Hz, 4H); ESI MS |
| | | (m/z): 411 [M + 1]. |
| 600 | N-(isobutyl(oxo)(2- | ¹H-NMR (400 MHz, DMSO-d₆) δ 9.50 (d, J = 1.0 Hz, 1H), 9.34 |
| | (pyridin-3-yl)-2H- | (d, J = 2.2 Hz, 1H), 8.71 (dd, J = 4.8, 1.3 Hz, 1H), 8.52-8.49 (m, |
| | indazol-5-yl)-λ⁶- | 2H), 7.98 (d, J = 9.0 Hz, 1H), 7.71-7.67 (m, 2H), 3.60 (dd, J = |
| | sulfaneylidene)cyclopro- | 14.5, 6.2 Hz, 1H), 3.51 (dd, J = 14.4, 6.4 Hz, 1H), 2.01 (t, J = 6.7 |
| | panecarboxamide | Hz, 1H), 1.61 (d, J = 6.6 Hz, 1H), 0.98 (d, J = 6.6 Hz, 3H), 0.89 |
| | | (d, J = 6.8 Hz, 3H), 0.78-0.74 (m, 4H); ESI MS (m/z): 383 |
| | | [M + 1]. |
| 601 | N-(oxo(pentan-2-yl)(2- | ¹H-NMR (400 MHz, CHLOROFORM-D) δ 9.32 (s, 1H), 8.77- |
| | (pyridin-3-yl)-2H- | 8.74 (m, 2H), 8.46-8.45 (m, 2H), 7.89 (d, J = 9.3 Hz, 1H), 7.68- |
| | indazol-5-yl)-λ⁶- | 7.62 (m, 2H), 3.59-3.49 (m, 1H), 2.21-2.16 (m, 1H), 1.82-1.77 |
| | sulfaneylidene)cyclopro- | (m, 1H), 1.48-1.23 (m, 6H), 1.02-0.98 (m, 2H), 0.96-0.80 (m, |
| | panecarboxamide | 5H); ESI MS (m/z): 397.05 [M + 1]. |
| 605 | N-(oxo(pentan-3-yl)(2- | ¹H-NMR (400 MHz, CHLOROFORM-D) δ 9.28 (s, 1H), 8.81- |
| | (pyridin-3-yl)-2H- | 8.73 (m, 2H), 8.48 (d, J = 1.0 Hz, 1H), 8.41 (d, J = 9.0 Hz, 1H), |
| | indazol-5-yl)-λ⁶- | 7.86 (dd, J = 15.0, 8.7 Hz, 1H), 7.67-7.58 (m, 2H), 3.28-3.22 (m, |
| | sulfaneylidene)cyclopro- | 1H), 2.19-2.08 (m, 1H), 1.92-1.75 (m, 3H), 1.68-1.57 (m, 1H), |
| | panecarboxamide | 1.13-1.07 (m, 3H), 1.04-0.94 (m, 5H), 0.85-0.77 (m, 2H) |
| | | ESI MS (m/z): 397.2 [M + 1]. |
| 606 | N-(cyclohexyl(oxo)(2- | ¹H-NMR (400 MHz, MeOD) δ 9.34 (d, J = 2.0 Hz, 1H), 9.27 (d, |
| | (pyridin-3-yl)-2H- | J = 1.0 Hz, 1H), 8.71 (dd, J = 4.9, 1.5 Hz, 1H), 8.57-8.53 (m, |
| | indazol-5-yl)-λ⁶- | 2H), 7.97 (d, J = 9.3 Hz, 1H), 7.73-7.69 (m, 2H), 4.60 (s, 1H), |
| | sulfaneylidene)acetamide | 3.51-3.45 (m, 1H), 2.35 (s, 1H), 2.14 (s, 3H), 1.94 (d, J = 15.2 |
| | | Hz, 2H), 1.86 (d, J = 11.7 Hz, 1H), 1.69 (s, 1H), 1.49-1.30 (m, |
| | | 4H); ESI MS (m/z): 382.9 [M + 1]. |
| 607 | N-(oxo(pentan-2-yl)(2- | ¹H-NMR (400 MHz, MeOD) δ 9.31 (d, J = 2.7 Hz, 1H), 9.25 (s, |
| | (pyridin-3-yl)-2H- | 1H), 8.68 (dd, J = 4.8, 1.3 Hz, 1H), 8.54-8.51 (m, 2H), 7.95-7.92 |
| | indazol-5-yl)-λ⁶-sul- | (m, 1H), 7.70-7.66 (m, 2H), 4.57 (s, 1H), 3.60-3.53 (m, 1H), |
| | faneylidene)propionamide | 2.44-2.38 (m, 2H), 2.16-1.80 (m, 1H), 1.41 (d, J = 6.8 Hz, 2H), |
| | | 1.24 (d, J = 6.8 Hz, 2H), 1.15-1.09 (m, 3H), 0.95 (t, J = 7.2 Hz, |
| | | 2H), 0.86 (t, J = 7.2 Hz, 2H); ESI MS (m/z): 385.05 [M + 1]. |
| 608 | N-(oxo(pentan-3-yl)(2- | ¹H-NMR (400 MHz, CHLOROFORM-D) δ 9.29 (d, J = 2.1 Hz, |
| | (pyridin-3-yl)-2H- | 1H), 8.76-8.74 (m, 2H), 8.49 (d, J = 0.6 Hz, 1H), 8.44 (d, J = 8.3 |
| | indazol-5-yl)-λ⁶-sul- | Hz, 1H), 7.88 (d, J = 9.2 Hz, 1H), 7.66-7.62 (m, 2H), 3.30-3.24 |
| | faneylidene)propionamide | (m, 1H), 2.44 (q, J = 7.5 Hz, 2H), 2.19-2.09 (m, 1H), 1.90-1.79 |
| | | (m, 2H), 1.68-1.57 (m, 1H), 1.17-1.07 (m, 6H), 0.95 (t, J = 7.5 |
| | | Hz, 3H); ESI MS (m/z): 384.95 [M + 1]. |
| 611 | N-(oxo(2-(pyridin-3-yl)- | ¹H-NMR (400 MHz, CHLOROFORM-D) δ 9.21 (d, J = 2.4 Hz, |
| | 2H-indazol-5-yl)(3,3,3- | 1H), 8.73-8.71 (m, 2H), 8.66 (d, J = 0.6 Hz, 1H), 8.30 (dq, J = |
| | trifluoropropyl)-λ⁶- | 8.3, 1.4 Hz, 1H), 7.89 (d, J = 9.2 Hz, 1H), 7.72 (dd, J = 9.3, 1.7 |
| | sulfaneylidene)acetamide | Hz, 1H), 7.55-7.52 (m, 1H), 4.16-4.11 (m, 1H), 4.00-3.94 (m, |
| | | 1H), 3.55-3.48 (m, 1H), 3.44-3.37 (m, 1H), 2.61-2.54 (m, 1H), |
| | | 2.41-2.34 (m, 1H); ESI MS (m/z): 396.85 [M + 1]. |
| 613 | N-(neopentyl(oxo)(2- | ¹H-NMR (400 MHz, METHANOL-D4) δ 9.30 (d, J = 2.4 Hz, |
| | (pyridin-3-yl)-2H- | 1H), 9.24 (s, 1H), 8.68 (dd, J = 4.7, 1.4 Hz, 1H), 8.59 (d, J = 1.2 |
| | indazol-5-yl)-λ⁶- | Hz, 1H), 8.52 (dq, J = 8.4, 1.3 Hz, 1H), 7.94 (d, J = 9.2 Hz, 1H), |
| | sulfaneylidene)acetamide | 7.76 (dd, J = 9.2, 1.8 Hz, 1H), 7.69 (dd, J = 8.3, 4.9 Hz, 1H), |
| | | 3.71 (d, J = 14.7 Hz, 1H), 3.47 (d, J = 14.7 Hz, 1H), 2.05 (d, J = |
| | | 14.7 Hz, 3H), 1.12 (t, J = 14.7 Hz, 9H); ESI MS (m/z): 370.95 |
| | | M + 1]. |
| 614 | N-(neopentyl(oxo)(2- | ¹H-NMR (400 MHz, METHANOL-D4) δ 9.30 (d, J = 2.4 Hz, |
| | (pyridin-3-yl)-2H- | 1H), 9.24 (s, 1H), 8.67 (dd, J = 4.9, 1.2 Hz, 1H), 8.56 (d, J = 1.5 |
| | indazol-5-yl)-λ⁶- | Hz, 1H), 8.52 (dq, J = 8.3, 1.3 Hz, 1H), 7.93 (d, J = 9.5 Hz, 1H), |
| | sulfaneylidene)cyclopro- | 7.74 (dd, J = 9.2, 1.8 Hz, 1H), 7.69 (dd, J = 8.3, 4.9 Hz, 1H), |
| | panecarboxamide | 3.68 (d, J = 14.4 Hz, 1H), 3.42 (d, J = 14.5 Hz, 1H), 1.70-1.65 |
| | | (m, 1H), 1.12 (d, J = 14.7 Hz, 9H), 0.98-0.81 (m, 4H); ESI MS |
| | | (m/z): 397.1 [M + 1]. |
| 616 | N-(oxo(propyl)(2- | ¹H-NMR (400 MHz, DMSO-d₆) δ 9.50 (d, J = 1.0 Hz, 1H), 9.34 |
| | (pyridin-3-yl)-2H- | (d, J = 2.2 Hz, 1H), 8.71 (dd, J = 4.6, 1.5 Hz, 1H), 8.53-8.49 (m, |
| | indazol-5-yl)-λ⁶- | 2H), 7.98 (d, J = 9.3 Hz, 1H), 7.71-7.67 (m, 2H), 3.66-3.52 (m, |
| | sulfaneylidene)acetamide | 2H), 2.00 (s, 3H), 1.64-1.44 (m, 2H), 0.89 (t, J = 7.5 Hz, 3H) |
| | | ESI MS (m/z): 343.05 [M + 1]. |

For fluorinated acyl analogues, corresponding acid anhydrides were used.

### Example 11: Synthesis of N-((cyclopropylmnethyl)(oxo)(2-(pyrimidin-5-yl)-2H-indazol-5-yl)-λ⁶-sulfaneylidene)-3,3-difluorocyclobutane-1-carboxamide 429

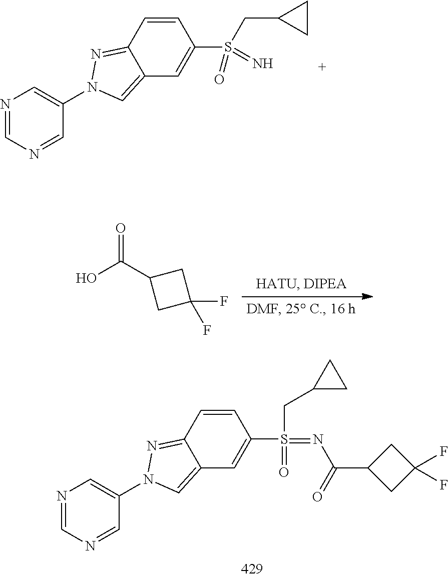 [SMILES: N=S(=O)(CC1CC1)c1ccc2nn(-c3cncnc3)cc2c1 ; O=C(O)C1CC(F)(F)C1 ; O=C(N=S(=O)(CC1CC1)c1ccc2nn(-c3cncnc3)cc2c1)C1CC(F)(F)C1]

To a stirred solution of (cyclopropylmethyl)(imino)(2-(pyrimidin-5-yl)-2H-indazol-5-yl)-λ⁶-sulfanone (250 mg, 0.798 mmol) in N,N-dimethylformamnide (3 mL), 3,3-difluorocyclobutane-1-carboxylic acid (130 mg, 0.957 mmol) and 1-[Bis(dimethylamino)methylene]-1H-1,2,3-triazolo[4,5-b]pyridinium 3-oxide hexafluorophosphate (455 mg, 1.197 mmol) were added at 25° C. Then, N,N-diisopropylethylamine (0.278 mL, 1.596 mmol) was added and the resulting mixture was stirred at 25° C. for 16 h. After completion of the reaction, the reaction mixture was quenched by addition of water (10 mL), extracted with ethyl acetate (3×25 mL) and washed with brine (1×25 mL). The organic layer was further dried over anhydrous sodium sulfate, filtered and concentrated under reduced pressure to obtain a crude gum. The crude compound was purified by preparative HPLC to get the desired N-((cyclopropylmethyl)(oxo)(2-(pyrimidin-5-yl)-2H-indazol-5-yl)-λ⁶-sulfaneylidene)-3,3-difluorocyclobutane-1-carboxamide 429 (88 mg, 0.204 mmol, 26% o yield).

TABLE 11
Representative compounds of the present disclosure were prepared according to
the suitable starting material and methods as described in the Example 11.

| Compd | | |
|---|---|---|
| no. | Compound name | Analytical Data |
| 420 | 1-cyano-N- | ¹H-NMR (400 MHz, DMSO-d₆) δ 9.58-9.56 (m, 3H), 9.33 (s, |
| | (isopropyl(oxo)(2- | 1H), 8.53 (d, J = 1.0 Hz, 1H), 8.03 (d, J = 9.3 Hz, 1H), 7.64 |
| | (pyrimidin-5-yl)-2H- | (dd, J = 9.2, 1.8 Hz, 1H), 3.91-3.85 (m, 1H), 1.64-1.46 (m, 4H), |
| | indazol-5-yl)-λ⁶- | 1.36 (d, J = 6.6 Hz, 3H), 1.17 (d, J = 6.6 Hz, 3H); ESI MS |
| | sulfaneylidene)cyclo- | (m/z): 394.6 [M + 1]. |
| | propane-1- | |
| | carboxamide | |
| 423 | N-(isopropyl(oxo)(2- | ¹H-NMR (400 MHz, DMSO-d₆) δ 9.57-9.56 (m, 3H), 9.32 (s, |
| | (pyrimidin-5-yl)-2H- | 1H), 8.42 (q, J = 0.9 Hz, 1H), 8.01-7.98 (m, 1H), 7.57 (dd, J = |
| | indazol-5-yl)-λ⁶- | 9.2, 1.8 Hz, 1H), 3.71 (q, J = 6.7 Hz, 1H), 1.28 (d, J = 6.8 Hz, |
| | sulfaneylidene)-1- | 3H), 1.21 (s, 3H), 1.15 (q, J = 2.8 Hz, 2H), 1.12 (d, J = 6.6 Hz, |
| | methylcyclopropane- | 3H), 0.65 (dd, J = 9.0, 6.1 Hz, 2H); ESI MS (m/z): 384.1 |
| | 1-carboxamide | [M + 1]. |
| 425 | 1-cyano-N-((2-(5- | ¹H-NMR (400 MHz, DMSO-d₆) δ 9.56 (d, J = 0.7 Hz, 1H), |
| | fluoropyridin-3-yl)- | 9.27 (s, 1H), 8.77 (d, J = 2.2 Hz, 1H), 8.56 (dt, J = 9.8, 2.4 Hz, |
| | 2H-indazol-5- | 1H), 8.50 (t, J = 0.9 Hz, 1H), 8.01 (d, J = 9.3 Hz, 1H), 7.63 (dd, |
| | yl)(isopropyl)(oxo)- | J = 9.3, 1.7 Hz, 1H), 3.91-3.85 (m, 1H), 1.62-1.46 (m, 4H), |
| | λ⁶- | 1.36 (d, J = 6.6 Hz, 3H), 1.17 (d, J = 6.6 Hz, 3H); ESI MS |
| | sulfaneylidene)cyclo- | (m/z): 412 [M + 1]. |
| | propane-1- | |
| | carboxamide | |
| 426 | 1-cyano-N-((cyclo- | ¹H-NMR (400 MHz, DMSO-d₆) δ 9.62 (d, J = 0.7 Hz, 1H), |
| | propylmethyl)(oxo)(2- | 9.56 (d, J = 12.0 Hz, 2H), 9.33 (s, 1H), 8.61 (t, J = 0.9 Hz, 1H), |
| | (pyrimidin-5- | 8.04 (d, J = 9.3 Hz, 1H), 7.75 (dd, J = 9.3, 1.7 Hz, 1H), 3.77 (t, |
| | yl)-2H-indazol-5-yl)- | J = 7.6 Hz, 2H), 1.65-1.49 (m, 4H), 0.88-0.82 (m, 1H), 0.50- |
| | λ⁶- | 0.40 (m, 2H), 0.23-0.09 (m, 2H); ESI MS (m/z): 406.9 [M + 1]. |
| | sulfaneylidene)cyclo- | |
| | propane-1- | |
| | carboxamide | |
| 427 | N-((cyclo- | ¹H-NMR (400 MHz, DMSO-d₆) δ 9.62 (d, J = 1.0 Hz, 1H), |
| | propylmethyl)(oxo)(2- | 9.57 (s, 2H), 9.33 (s, 1H), 8.57 (d, J = 1.0 Hz, 1H), 8.03 (d, J = |
| | (pyrimidin-5- | 9.3 Hz, 1H), 7.71 (dd, J = 9.3, 2.0 Hz, 1H), 3.79-3.68 (m, 2H), |
| | yl)-2H-indazol-5-yl)- | 1.37 (d, J = 8.6 Hz, 6H), 0.83 (t, J = 7.6 Hz, 1H), 0.44-0.39 (m, |
| | λ⁶-sulfaneylidene)- | 2H), 0.18-0.08 (m, 2H); ESI MS (m/z): 452.1 [M + 1]. |
| | 3,3,3-trifluoro-2,2- | |
| | dimethylpropanamide | |
| 428 | N-((cyclo- | ¹H-NMR (400 MHz, DMSO-d₆) δ 9.59 (d, J = 1.0 Hz, 1H), |
| | propylmethyl)(oxo)(2- | 9.56 (s, 2H), 9.32 (s, 1H), 8.54 (t, J = 0.9 Hz, 1H), 7.99 (d, J = |
| | (pyrimidin-5- | 9.3 Hz, 1H), 7.71 (dd, J = 9.3, 1.7 Hz, 1H), 3.65 (dq, J = 23.5, |
| | yl)-2H-indazol-5-yl)- | 7.3 Hz, 2H), 2.25-2.19 (m, 1H), 1.86 (d, J = 4.2 Hz, 2H), 1.70- |
| | λ⁶- | 1.58 (m, 3H), 1.38-1.13 (m, 5H), 0.81 (t, J = 7.3 Hz, 1H), 0.45- |
| | sulfaneylidene)cyclo- | 0.35 (m, 2H), 0.13-0.03 (m, 2H); ESI MS (m/z): 424.2 [M + 1]. |
| | hexanecarboxamide | |
| 429 | N-((cyclo- | ¹H-NMR (400 MHz, DMSO-d₆) δ 9.59 (d, J = 1.0 Hz, 1H), |
| | propylmethyl)(oxo)(2- | 9.57 (s, 2H), 9.33 (s, 1H), 8.58 (d, J = 1.0 Hz, 1H), 8.01 (d, J = |
| | (pyrimidin-5- | 9.3 Hz, 1H), 7.73 (dd, J = 9.3, 1.7 Hz, 1H), 3.72 (d, J = 7.1 Hz, |
| | yl)-2H-indazol-5-yl)- | 2H), 3.03-2.99 (m, 1H), 2.84-2.74 (m, 4H), 0.83 (t, J = 7.5 Hz, |
| | λ⁶-sulfaneylidene)- | 1H), 0.47-0.37 (m, 2H), 0.16 (q, J = 4.7 Hz, 1H), 0.07-0.03 (m, |
| | 3,3- | 1H); ESI MS (m/z): 431.9 [M + 1]. |
| | difluorocyclobutane- | |
| | 1-carboxamide | |
| 430 | N-((cyclo- | ¹H-NMR (400 MHz, DMSO-d₆) δ 9.58-9.55 (m, 3H), 9.32 (d, |
| | propylmethyl)(oxo)(2- | J = 4.2 Hz, 1H), 8.55 (t, J = 0.9 Hz, 1H), 7.99 (d, J = 9.3 Hz, 1H), |
| | (pyrimidin-5- | 7.72 (dd, J = 9.2, 1.8 Hz, 1H), 3.66 (d, J = 7.3 Hz, 2H), 2.26 (t, |
| | yl)-2H-indazol-5-yl)- | J = 7.2 Hz, 2H), 1.54 (td, J = 14.7, 7.4 Hz, 2H), 0.92-0.80 (m, |
| | λ⁶-sul- | 4H), 0.47-0.34 (m, 2H), 0.13 (td, J = 9.3, 4.9 Hz, 1H), 0.07- |
| | faneylidene)butyramide | 0.02 (m, 1H); ESI MS (m/z): 383.95 [M + 1]. |
| 431 | N-((cyclo- | ¹H-NMR (400 MHz, DMSO-d₆) δ 9.60 (d, J = 1.0 Hz, 1H), |
| | propylmethyl)(oxo)(2- | 9.57 (s, 2H), 9.32 (d, J = 4.2 Hz, 1H), 8.58 (t, J = 0.9 Hz, 1H), |
| | (pyrimidin-5- | 8.02 (d, J = 9.3 Hz, 1H), 7.72 (dd, J = 9.3, 2.0 Hz, 1H), 3.72 (d, |
| | yl)-2H-indazol-5-yl)- | J = 7.3 Hz, 2H), 1.36-1.30 (m, 4H), 0.86-0.80 (m, 1H), 0.50- |
| | λ⁶-sulfaneylidene)-1- | 0.37 (m, 2H), 0.17 (td, J = 9.4, 4.8 Hz, 1H), 0.07 (td, J = 9.4, |
| | fluorocyclopropane-1- | 4.8 Hz, 1H); ESI MS (m/z): 399.9 [M + 1]. |
| | carboxamide | |
| 432 | N-((cyclo- | ¹H-NMR (400 MHz, DMSO-d₆) δ 9.60 (t, J = 1.2 Hz, 1H), 9.57 |
| | propylmethyl)(oxo)(2- | (d, J = 4.2 Hz, 2H), 9.33-9.30 (m, 1H), 8.58 (dq, J = 5.9, 0.8 |
| | (pyrimidin-5- | Hz, 1H), 8.01 (dd, J = 9.0, 3.7 Hz, 1H), 7.73 (dt, J = 9.2, 1.5 |
| | yl)-2H-indazol-5-yl)- | Hz, 1H), 3.78-3.69 (m, 2H), 2.77-2.67 (m, 1H), 1.94-1.82 (m, |
| | λ⁶-sulfaneylidene)- | 2H), 0.87-0.81 (m, 1H), 0.49-0.36 (m, 2H), 0.19-0.12 (m, 1H), |
| | 2,2- | 0.10-0.03 (m, 1H); ESI MS (m/z): 417.9 [M + 1]. |
| | difluorocyclopropane- | |
| | 1-carboxamide | |
| 433 | N-((cyclo- | ¹H-NMR (400 MHz, DMSO-d₆) δ 9.58 (s, 1H), 9.56 (d, J = 0.7 |
| | propylmethyl)(oxo)(2- | Hz, 2H), 9.32 (d, J = 4.4 Hz, 1H), 8.56 (t, J = 0.9 Hz, 1H), 8.01- |
| | (pyrimidin-5- | 7.99 (m, 1H), 7.73 (dd, J = 9.3, 1.0 Hz, 1H), 4.98 (s, 1H), 3.69 |
| | yl)-2H-indazol-5-yl)- | (d, J = 7.1 Hz, 2H), 2.17-2.08 (m, 1H), 1.49-1.41 (m, 1H), 1.19 |
| | λ⁶-sulfaneylidene)-2- | (dt, J = 18.2, 6.4 Hz, 1H), 0.83 (s, 1H), 0.48-0.38 (m, 2H), 0.15 |
| | fluorocyclopropane-1- | (dd, J = 8.8, 5.1 Hz, 1H), 0.07 (s, 1H); ESI MS (m/z): 399.85 |
| | carboxamide | [M + 1]. |
| 434 | N-((cyclo- | ¹H-NMR (400 MHz, DMSO-d₆) δ 9.59 (d, J = 1.0 Hz, 1H), |
| | propylmethyl)(oxo)(2- | 9.57 (s, 2H), 9.32 (d, J = 4.4 Hz, 1H), 8.53 (d, J = 1.0 Hz, 1H), |
| | (pyrimidin-5- | 8.00 (d, J = 9.3 Hz, 1H), 7.71 (qd, J = 4.6, 1.9 Hz, 1H), 3.66 (d, |
| | yl)-2H-indazol-5-yl)- | J = 7.1 Hz, 2H), 2.98-2.89 (m, 1H), 2.32-2.29 (m, 1H), 2.27- |
| | λ⁶-sulfaneylidene)-3- | 2.20 (m, 2H), 1.77-1.68 (m, 2H), 1.08 (d, J = 6.4 Hz, 1H), 0.98 |
| | methylcyclobutane-1- | (d, J = 5.9 Hz, 2H), 0.83-0.78 (m, 1H), 0.47-0.34 (m, 2H), 0.17- |
| | carboxamide | 0.11 (m, 1H), 0.07-0.02 (m, 1H); ESI MS (m/z): 410 [M + 1]. |
| 435 | N-((cyclo- | ¹H-NMR (400 MHz, DMSO-d₆) δ 9.62 (d, J = 0.7 Hz, 1H), |
| | propylmethyl)(oxo)(2- | 9.57 (s, 2H), 9.33 (s, 1H), 8.63 (q, J = 0.8 Hz, 1H), 8.05 (d, J = |
| | (pyrimidin-5- | 9.3 Hz, 1H), 7.74 (dd, J = 9.3, 2.0 Hz, 1H), 3.91-3.81 (m, 2H), |
| | yl)-2H-indazol-5-yl)- | 2.15-2.01 (m, 2H), 0.94 (t, J = 7.5 Hz, 3H), 0.89-0.82 (m, 1H), |
| | λ⁶-sulfaneylidene)- | 0.52-0.38 (m, 2H), 0.23 (td, J = 9.5, 4.8 Hz, 1H), 0.09 (td, J = |
| | 2,2- | 9.7, 4.5 Hz, 1H); ESI MS (m/z): 419.95 [M + 1]. |
| | difluorobutanamide | |
| 436 | N-((cyclo- | ¹H-NMR (400 MHz, DMSO-d₆) δ 9.59 (d, J = 1.0 Hz, 1H), |
| | propylmethyl)(oxo)(2- | 9.56 (s, 2H), 9.32 (d, J = 4.2 Hz, 1H), 8.54 (q, J = 0.8 Hz, 1H), |
| | (pyrimidin-5- | 8.00 (d, J = 9.3 Hz, 1H), 7.71 (dd, J = 9.3, 1.7 Hz, 1H), 3.71- |
| | yl)-2H-indazol-5-yl)- | 3.61 (m, 2H), 2.77-2.69 (m, 1H), 1.82-1.70 (m, 4H), 1.61-1.48 |
| | λ⁶- | (m, 4H), 0.84-0.78 (m, 1H), 0.46-0.34 (m, 2H), 0.15-0.03 (m, |
| | sulfaneylidene)cyclo- | 2H); ; ESI MS (m/z): 409.9 [M + 1]. |
| | pentanecarboxamide | |
| 437 | N-((cyclo- | ¹H-NMR (400 MHz, DMSO-d₆) δ 9.57 (dd, J = 6.9, 1.1 Hz, |
| | propylmethyl)(oxo)(2- | 3H), 9.32 (d, J = 1.5 Hz, 1H), 8.53 (qd, J = 1.9, 0.9 Hz, 1H), |
| | (pyrimidin-5- | 8.01-7.96 (m, 1H), 7.71 (dt, J = 9.2, 1.7 Hz, 1H), 3.65 (d, J = |
| | yl)-2H-indazol-5-yl)- | 7.0 Hz, 2H), 1.67-1.62 (m, 1H), 1.13 (d, J = 2.8 Hz, 3H), 1.08 |
| | λ⁶-sulfaneylidene)- | (d, J = 22.9 Hz, 3H), 0.86-0.81 (m, 2H), 0.72 (q, J = 3.8 Hz, |
| | 2,2- | 1H), 0.46-0.35 (m, 2H), 0.15-0.11 (m, 1H), 0.03 (q, J = 4.7 Hz, |
| | dimethylcyclopropane- | 1H); ESI MS (m/z): 410 [M + 1]. |
| | 1-carboxamide | |
| 438 | 3-cyano-N-((cyclo- | ¹H-NMR (400 MHz, DMSO-d₆) δ 9.58-9.53 (m, 3H), 9.32 (d, |
| | propylmethyl)(oxo)(2- | J = 4.6 Hz, 1H), 8.60 (t, J = 0.9 Hz, 1H), 7.99 (d, J = 9.3 Hz, 1H), |
| | (pyrimidin-5- | 7.76 (dd, J = 9.2, 1.8 Hz, 1H), 3.75-3.65 (m, 2H), 2.69-2.60 (m, |
| | yl)-2H-indazol-5-yl)- | 4H), 0.90-0.83 (m, 1H), 0.47-0.36 (m, 2H), 0.16-0.04 (m, 2H) |
| | λ⁶- | ESI MS (m/z): 394.75 [M + 1]. |
| | sulfaneylidene)pro- | |
| | panamide | |
| 439 | N-((cyclo- | ¹H-NMR (400 MHz, DMSO-d₆) δ 9.60 (d, J = 1.0 Hz, 1H), |
| | propylmethyl)(oxo)(2- | 9.57 (s, 2H), 9.32 (d, J = 4.4 Hz, 1H), 8.58 (q, J = 0.8 Hz, 1H), |
| | (pyrimidin-5- | 8.01 (d, J = 9.3 Hz, 1H), 7.77-7.73 (m, 1H), 3.71-3.63 (m, 2H), |
| | yl)-2H-indazol-5-yl)- | 3.28-3.23 (m, 2H), 3.06-3.00 (m, 1H), 1.25-1.14 (m, 6H), 0.89- |
| | λ⁶-sulfaneylidene)-2- | 0.82 (m, 1H), 0.47-0.35 (m, 2H), 0.17-0.03 (m, 2H); ESI MS |
| | (isopropylthio)acetamide | (m/z): 430.1 [M + 1]. |
| 440 | N-((cyclo- | ¹H-NMR (400 MHz, DMSO-d₆) δ 9.59 (d, J = 0.7 Hz, 1H), |
| | propylmethyl)(oxo)(2- | 9.57 (s, 2H), 9.32 (d, J = 4.4 Hz, 1H), 8.58 (d, J = 0.7 Hz, 1H), |
| | (pyrimidin-5- | 8.00 (d, J = 9.3 Hz, 1H), 7.75 (dd, J = 9.3, 1.7 Hz, 1H), 3.73- |
| | yl)-2H-indazol-5-yl)- | 3.63 (m, 2H), 3.22 (dd, J = 18.6, 13.9 Hz, 2H), 2.47 (d, J = 6.8 |
| | λ⁶-sulfaneylidene)-2- | Hz, 2H), 1.82-1.72 (m, 1H), 0.94-0.83 (m, 7H), 0.47-0.35 (m, |
| | (isobutylthio)acetamide | 2H), 0.10 (tq, J = 18.1, 4.8 Hz, 2H); ESI MS (m/z): 444.3 |
| | | [M + 1]. |
| 441 | N-((cyclo- | ¹H-NMR (400 MHz, DMSO-d₆) δ 9.59 (d, J = 0.7 Hz, 1H), |
| | propylmethyl)(oxo)(2- | 9.56 (s, 2H), 9.32 (s, 1H), 8.52 (d, J = 1.0 Hz, 1H), 8.00 (d, J = |
| | (pyrimidin-5- | 9.3 Hz, 1H), 7.70 (dd, J = 9.2, 1.8 Hz, 1H), 3.67-3.58 (m, 2H), |
| | yl)-2H-indazol-5-yl)- | 1.25-1.20 (m, 3H), 1.16 (d, J = 2.9 Hz, 2H), 0.82-0.77 (m, 1H), |
| | λ⁶-sulfaneylidene)-1- | 0.70-0.64 (m, 2H), 0.47-0.36 (m, 2H), 0.15-0.04 (m, 2H) |
| | methylcyclopropane- | ESI MS (m/z): 396.15 [M + 1]. |
| | 1-carboxamide | |
| 442 | 1-cyano-N-((cyclo- | ¹H-NMR (400 MHz, DMSO-d₆) δ 9.63 (d, J = 1.0 Hz, 1H), |
| | propylmethyl)(oxo)(2- | 9.57 (s, 2H), 9.33 (s, 1H), 8.63 (t, J = 0.9 Hz, 1H), 8.05 (d, J = |
| | (pyrimidin-5- | 9.3 Hz, 1H), 7.76 (dd, J = 9.3, 2.0 Hz, 1H), 3.82 (d, J = 7.1 Hz, |
| | yl)-2H-indazol-5-yl)- | 2H), 2.66-2.52 (m, 4H), 2.15-1.96 (m, 2H), 0.87-0.82 (m, 1H), |
| | λ⁶- | 0.50-0.39 (m, 2H), 0.21 (td, J = 9.2, 4.9 Hz, 1H), 0.14-0.08 (m, |
| | sulfaneylidene)cyclob | 1H); ESI MS (m/z): 421 [M + 1]. |
| | utane-1-carboxamide | |
| 443 | N-((cyclo- | ¹H-NMR (400 MHz, DMSO-d₆) δ 9.59 (d, J = 1.0 Hz, 1H), |
| | propylmethyl)(oxo)(2- | 9.57 (s, 2H), 9.32 (d, J = 4.4 Hz, 1H), 8.54 (q, J = 0.9 Hz, 1H), |
| | (pyrimidin-5- | 7.99 (d, J = 9.3 Hz, 1H), 7.70 (dd, J = 9.2, 1.8 Hz, 1H), 3.66 (d, |
| | yl)-2H-indazol-5-yl)- | J = 7.1 Hz, 2H), 3.11-3.02 (m, 1H), 1.98-1.85 (m, 4H), 1.13 (s, |
| | λ⁶-sulfaneylidene)- | 3H), 1.02 (t, J = 12.2 Hz, 3H), 0.83-0.79 (m, 1H), 0.45-0.36 (m, |
| | 3,3- | 2H), 0.16-0.11 (m, 1H), 0.07-0.02 (m, 1H); ESI MS (m/z): |
| | dimethylcyclobutane- | 424.4 [M + 1]. |
| | 1-carboxamide | |
| 444 | N-((cyclo- | ¹H-NMR (400 MHz, DMSO-d₆) δ 9.60 (d, J = 0.7 Hz, 1H), |
| | propylmethyl)(oxo)(2- | 9.57 (s, 2H), 9.32 (d, J = 4.4 Hz, 1H), 8.54 (q, J = 0.9 Hz, 1H), |
| | (pyrimidin-5- | 8.00 (d, J = 9.3 Hz, 1H), 7.72 (dd, J = 9.3, 1.7 Hz, 1H), 3.73- |
| | yl)-2H-indazol-5-yl)- | 3.62 (m, 2H), 2.44-2.36 (m, 2H), 1.93-1.84 (m, 1H), 1.76-1.69 |
| | λ⁶-sulfaneylidene)-1- | (m, 3H), 1.36 (d, J = 9.8 Hz, 3H), 0.83-0.78 (m, 1H), 0.46-0.36 |
| | methylcyclobutane-1- | (m, 2H), 0.15-0.05 (m, 2H); ESI MS (m/z): 410.45 [M + 1]. |
| | carboxamide | |
| 445 | N-((2-(5- | ¹H-NMR (400 MHz, DMSO-d₆) δ 9.55 (d, J = 0.9 Hz, 1H), |
| | fluoropyridin-3-yl)- | 9.27 (s, 1H), 8.76 (d, J = 2.8 Hz, 1H), 8.56 (dt, J = 9.8, 2.3 Hz, |
| | 2H-indazol-5- | 1H), 8.41 (d, J = 0.9 Hz, 1H), 7.97 (d, J = 9.2 Hz, 1H), 7.58 |
| | yl)(isopropyl)(oxo)- | (dd, J = 9.3, 1.7 Hz, 1H), 3.79-3.72 (m, 1H), 2.22 (qd, J = 7.2, |
| | λ⁶- | 3.6 Hz, 1H), 1.89-1.82 (m, 2H), 1.68 (t, J = 4.4 Hz, 2H), 1.59 |
| | sulfaneylidene)cyclo- | (d, J = 11.6 Hz, 1H), 1.37-1.12 (m, 11H); ESI MS (m/z): 429.3 |
| | hexanecarboxamide | [M + 1]. |
| 449 | N-((cyclo- | ¹H-NMR (400 MHz, DMSO-d₆) δ 9.58 (s, 1H), 9.28 (s, 1H), |
| | propylmethyl)(2- | 8.76 (d, J = 2.4 Hz, 1H), 8.58-8.54 (m, 1H), 8.49 (s, 1H), 7.98 |
| | (5-fluoropyridin-3- | (d, J = 9.0 Hz, 1H), 7.69 (dd, J = 9.3, 1.7 Hz, 1H), 3.63-3.61 |
| | yl)-2H-indazol-5- | (m, 2H), 1.24 (d, J = 8.8 Hz, 3H), 1.16 (d, J = 2.9 Hz, 2H), 0.79 |
| | yl)(oxo)-λ⁶- | (t, J = 7.6 Hz, 1H), 0.65 (d, J = 3.2 Hz, 2H), 0.44-0.40 (m, 2H), |
| | sulfaneylidene)-1- | 0.14-0.06 (m, 2H); ESI MS (m/z): 413 [M + 1]. |
| | methylcyclopropane- | |
| | 1-carboxamide | |

### Example 12: Synthesis of (2-(5-fluoropyridin-3-yl)-2H-indazol-5-yl)(isopropyl)(methylimino)-λ⁶-sulfanone 407

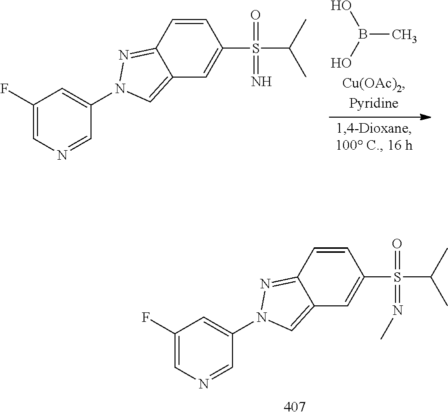

To a de-gased stirred solution of (2-(5-fluoropyridin-3-yl)-2H-indazol-5-yl)(imino)(isopropyl)-λ⁶-sulfanone (300 mg, 0.942 mmol) in 1,4-dioxane (15 mL), copper(II) acetate (257 mg, 1.413 mmol), pyridine (0.183 mL, 2.262 mmol) and methylboronic acid (282 mg, 4.71 mmol) were added. The resulting mixture was stirred under reflux condition (100° C.) for 16 h. After completion of the reaction, 1,4-dioxane was removed under reduced pressure and the reaction mixture was diluted with ethyl acetate (60 mL). The organic layer was extracted with water (3×20 mL), washed with brine (1×25 mL) and dried over anhydrous sodium sulfate. It was then concentrated under reduced pressure to obtain a crude solid. The crude material was further purified by preparative HPLC to get the desired 2-(5-fluoropyridin-3-yl)-2H-indazol-5-yl)(isopropyl)(methylimino)-λ⁶-sulfanone 407 (175 mg, 0.526 mmol, 56% yield).

TABLE 12
Representative compounds of the present disclosure were prepared according to
the suitable starting material and methods as described in the Example 12.

| Compd | | |
|---|---|---|
| no. | Compound name | Analytical Data |
| 405 | (cyclopropylimino)(2- | ¹H-NMR (400 MHz, DMSO-d₆) δ 9.50 (d, J = 0.7 Hz, 1H), |
| | (5-fluoropyridin-3- | 9.26 (s, 1H), 8.75 (d, J = 2.4 Hz, 1H), 8.54 (dt, J = 9.8, 2.3 Hz, |
| | yl)-2H-indazol-5- | 1H), 8.38 (s, 1H), 7.93 (d, J = 9.0 Hz, 1H), 7.63 (dd, J = 9.0, 1.7 |
| | yl)(isopropyl)-λ⁶- | Hz, 1H), 3.44-3.38 (m, 1H), 2.33-2.28 (m, 1H), 1.24 (d, J = 6.6 |
| | sulfanone | Hz, 3H), 1.07 (d, J = 6.6 Hz, 3H), 0.41-0.34 (m, 2H), 0.32-0.21 |
| | | (m, 2H); ESI MS (m/z): 358.55 [M + 1]. |
| 406 | (cyclopropylimino)(iso- | ¹H-NMR (400 MHz, DMSO-d₆) δ 9.46 (s, 1H), 9.33 (d, J = 2.4 |
| | propyl)(2-(pyridin- | Hz, 1H), 8.70 (dd, J = 4.6, 1.2 Hz, 1H), 8.50 (dq, J = 8.3, 1.4 |
| | 3-yl)-2H-indazol-5- | Hz, 1H), 8.38 (s, 1H), 7.93 (d, J = 9.2 Hz, 1H), 7.68 (q, J = 4.3 |
| | yl)-λ⁶-sulfanone | Hz, 1H), 7.62 (dd, J = 9.2, 1.8 Hz, 1H), 3.43-3.37 (m, 1H), |
| | | 2.32-2.28 (m, 1H), 1.24 (d, J = 6.7 Hz, 3H), 1.07 (d, J = 6.7 Hz, |
| | | 3H), 0.41-0.34 (m, 2H), 0.32-0.22 (m, 2H); ESI MS (m/z): |
| | | 341.35 [M + 1]. |
| 407 | (2-(5-fluoropyridin-3- | ¹H-NMR (400 MHz, DMSO-d₆) δ 9.50 (d, J = 0.9 Hz, 1H), |
| | yl)-2H-indazol-5- | 9.26 (q, J = 0.7 Hz, 1H), 8.75 (d, J = 3.1 Hz, 1H), 8.55 (dt, J = |
| | yl)(isopropyl)(methyl- | 9.8, 2.3 Hz, 1H), 8.31 (q, J = 0.8 Hz, 1H), 7.93 (dd, J = 9.2, 0.9 |
| | imino)-λ⁶-sulfanone | Hz, 1H), 7.57 (dd, J = 9.2, 1.5 Hz, 1H), 3.43-3.36 (m, 1H), 2.53 |
| | | (s, 3H), 1.25 (d, J = 6.7 Hz, 3H), 1.10 (d, J = 7.0 Hz, 3H) |
| | | ESI MS (m/z): 333.1 [M + 1]. |
| 408 | isopropyl(methylimi- | ¹H-NMR (400 MHz, DMSO-d₆) δ 9.45 (d, J = 0.7 Hz, 1H), |
| | no)(2-(pyridin-3-yl)- | 9.33 (d, J = 2.2 Hz, 1H), 8.70 (dd, J = 4.8, 1.3 Hz, 1H), 8.50 |
| | 2H-indazol-5-yl)-λ⁶- | (dq, J = 8.3, 1.4 Hz, 1H), 8.31 (q, J = 0.8 Hz, 1H), 7.94 (d, J = |
| | sulfanone | 9.0 Hz, 1H), 7.68 (ddd, J = 8.3, 4.6, 0.7 Hz, 1H), 7.56 (dd, J = |
| | | 9.0, 1.7 Hz, 1H), 3.42-3.35 (m, 1H), 2.53 (s, 3H), 1.26-1.22 (m, |
| | | 3H), 1.11 (d, J = 6.8 Hz, 3H); ESI MS (m/z): 315.35 [M + 1]. |
| 412 | (cyclopropylimino)(cyclo- | ¹H-NMR (400 MHz, DMSO-d₆) δ 9.51 (d, J = 0.7 Hz, 1H), |
| | propylmethyl)(2- | 9.27 (s, 1H), 8.74 (d, J = 2.7 Hz, 1H), 8.55 (dt, J = 9.8, 2.3 Hz, |
| | (5-fluoropyridin-3- | 1H), 8.43 (q, J = 0.8 Hz, 1H), 7.92 (d, J = 9.0 Hz, 1H), 7.69 |
| | yl)-2H-indazol-5-yl)- | (dd, J = 9.3, 1.7 Hz, 1H), 3.26 (d, J = 7.1 Hz, 2H), 2.35-2.30 |
| | λ⁶-sulfanone | (m, 1H), 0.90-0.83 (m, 1H), 0.44-0.35 (m, 3H), 0.34-0.22 (m, |
| | | 3H), 0.12 (td, J = 9.7, 4.8 Hz, 1H), −0.06 (dt, J = 14.9, 4.6 Hz, |
| | | 1H); ESI MS (m/z): 371 [M + 1]. |
| 414 | (cyclopropylmethyl)(2- | ¹H-NMR (400 MHz, DMSO-d₆) δ 9.50 (d, J = 1.0 Hz, 1H), |
| | (5-fluoropyridin-3- | 9.27 (s, 1H), 8.75 (q, J = 2.8 Hz, 1H), 8.55 (dt, J = 9.9, 2.3 Hz, |
| | yl)-2H-indazol-5- | 1H), 8.38 (q, J = 0.8 Hz, 1H), 7.92 (d, J = 9.3 Hz, 1H), 7.64 |
| | yl)(methylimino)-λ⁶- | (dd, J = 9.0, 1.7 Hz, 1H), 3.26-3.24 (m, 2H), 2.54 (s, 3H), 0.93- |
| | sulfanone | 0.86 (m, 1H), 0.45-0.30 (m, 2H), 0.04 (dtd, J = 55.2, 9.6, 4.8 |
| | | Hz, 2H); ESI MS (m/z): 344.9 [M + 1]. |
| 446 | (cyclopropylimino)(cyclo- | ¹H-NMR (400 MHz, DMSO-d₆) δ 9.55 (s, 2H), 9.54-9.53 (m, |
| | propylmethyl)(2- | 1H), 9.31 (s, 1H), 8.46 (s, 1H), 7.94 (d, J = 9.2 Hz, 1H), 7.69 |
| | (pyrimidin-5-yl)-2H- | (dd, J = 9.2, 1.5 Hz, 1H), 3.26 (d, J = 7.3 Hz, 2H), 2.36-2.31 |
| | indazol-5-yl)-λ⁶- | (m, 1H), 0.90-0.84 (m, 1H), 0.44-0.21 (m, 6H), 0.11 (td, J = |
| | sulfanone | 9.7, 4.8 Hz, 1H), −0.07 (td, J = 9.7, 4.9 Hz, 1H); ESI MS (m/z): |
| | | 354 [M + 1]. |
| 447 | (cyclopropylmethyl)(meth- | ¹H-NMR (400 MHz, DMSO-d₆) δ 9.55 (d, J = 4.3 Hz, 2H), |
| | ylimino)(2- | 9.52 (d, J = 0.6 Hz, 1H), 9.31-9.29 (m, 1H), 8.40 (d, J = 0.9 Hz, |
| | (pyrimidin-5-yl)-2H- | 1H), 7.93 (q, J = 4.7 Hz, 1H), 7.65 (dd, J = 9.2, 1.5 Hz, 1H), |
| | indazol-5-yl)-λ⁶- | 3.27-3.24 (m, 2H), 2.53 (d, J = 3.7 Hz, 3H), 0.94-0.87 (m, 1H), |
| | sulfanone | 0.45-0.30 (m, 2H), 0.11 (td, J = 9.5, 4.9 Hz, 1H), −0.03 (td, J = |
| | | 9.6, 4.8 Hz, 1H); ESI MS (m/z): 328.1 [M + 1]. |

### Example 13: Synthesis of (cyclobutylmethyl)(ethylimino)(2-(5-fluoropyridin-3-yl)-2H-indazol-5-yl)-λ⁶-sulfanone 471

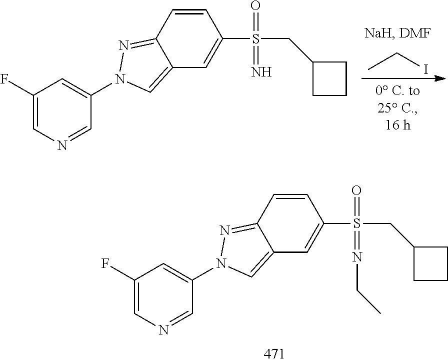

To a stirred solution of (cyclobutylmethyl)(2-(5-fluoropyridin-3-yl)-2H-indazol-5-yl)(imino)-λ⁶-5 sulfanone (250 mg, 0.726 mmol) in N,N-dimethylformamide (6 mL), sodium hydride (60% dispersion in mineral oil, 58.1 mg, 1.452 mmol) was added under nitrogen atmosphere at 0° C. and the resulting mixture was further stirred at 25° C. for 15 minutes. After that, it was cooled to 0° C. and 1-iodoethane (0.117 mL, 1.452 mmol) was further added at the same temperature. The resulting reaction mixture was stirred at 25° C. for 16 h. After completion of the reaction, the reaction mass was poured into water, and the aqueous layer was extracted with ethyl acetate (3×20 mL). The organic layer was separated, dried over anhydrous sodium sulfate, filtered, and concentrated under reduced pressure to obtain a crude solid. The crude material was purified by preparative HPLC to get the desired (cyclobutylmethyl)(ethylimino)(2-(5-fluoropyridin-3-yl)-2H-indazol-5-yl)-λ⁶-sulfanone 471 (167 mg, 0.448 mmol, 62% yield).

TABLE 13
Representative compounds of the present disclosure were prepared according to
the suitable starting material and methods as described in the Example 13.

| Compd | | |
|---|---|---|
| no. | Compound name | Analytical Data |
| 382 | (ethylimino)(isoprop- | ¹H-NMR (400 MHz, DMSO-d₆) δ 9.33 (d, J = 2.4 Hz, 1H), 9.16 |
| | yl)(2-(pyridin-3-yl)- | (d, J = 0.9 Hz, 1H), 8.69 (dd, J = 4.7, 1.4 Hz, 1H), 8.51 (dq, J = |
| | 2H-indazol-4-yl)-λ⁶- | 8.3, 1.4 Hz, 1H), 8.05 (d, J = 8.6 Hz, 1H), 7.66-7.60 (m, 2H), |
| | sulfanone | 7.54 (dd, J = 8.6, 7.0 Hz, 1H), 3.56-3.49 (m, 1H), 2.99-2.92 (m, |
| | | 1H), 2.89-2.83 (m, 1H), 1.32 (t, J = 7.0 Hz, 3H), 1.11-1.02 (m, |
| | | 6H); ESI MS (m/z): 329.15 [M + 1]. |
| 383 | (ethylimino)(isopro- | ¹H-NMR (400 MHz, DMSO-d₆) δ 9.45 (d, J = 0.7 Hz, 1H), 9.33 |
| | pyl)(2-(pyridin-3-yl)- | (d, J = 2.4 Hz, 1H), 8.70 (dd, J = 4.8, 1.3 Hz, 1H), 8.50 (dq, J = |
| | 2H-indazol-5-yl)-λ⁶- | 8.3, 1.4 Hz, 1H), 8.32 (s, 1H), 7.93 (d, J = 9.3 Hz, 1H), 7.68 (dd, |
| | sulfanone | J = 8.3, 4.6 Hz, 1H), 7.58 (dd, J = 9.2, 1.6 Hz, 1H), 3.41-3.34 |
| | | (m, 1H), 2.94-2.86 (m, 1H), 2.84-2.75 (m, 1H), 1.26-1.22 (m, |
| | | 3H), 1.10-1.05 (m, 6H); ESI MS (m/z): 328.55 [M + 1]. |
| 388 | (cyclopropylmethyl)(eth- | ¹H-NMR (400 MHz, DMSO-d₆) δ 9.46 (s, 1H), 9.34 (s, 1H), |
| | ylimino)(2- | 8.70 (d, J = 4.6 Hz, 1H), 8.50 (d, J = 7.1 Hz, 1H), 8.38 (s, 1H), |
| | (pyridin-3-yl)-2H- | 7.91 (d, J = 9.0 Hz, 1H), 7.70-7.64 (m, 2H), 3.24 (d, J = 7.1 Hz, |
| | indazol-5-yl)-λ⁶- | 2H), 2.94-2.79 (m, 2H), 1.07 (t, J = 7.2 Hz, 3H), 0.87 (d, J = 7.6 |
| | sulfanone | Hz, 1H), 0.43-0.38 (m, 1H), 0.33 (dd, J = 13.8, 4.5 Hz, 1H), |
| | | 0.14-0.09 (m, 1H), −0.02-−0.07 (m, 1H) |
| | | ESI MS (m/z): 341.05 [M + 1]. |
| 390 | butyl(ethylimino)(2- | ¹H-NMR (400 MHz, DMSO-d₆) δ 9.45 (d, J = 0.9 Hz, 1H), 9.33 |
| | (pyridin-3-yl)-2H- | (d, J = 2.4 Hz, 1H), 8.70 (dd, J = 4.7, 1.4 Hz, 1H), 8.50 (dq, J = |
| | indazol-5-yl)-λ⁶- | 8.3, 1.4 Hz, 1H), 8.36 (d, J = 0.9 Hz, 1H), 7.93 (d, J = 9.2 Hz, |
| | sulfanone | 1H), 7.70-7.66 (m, 1H), 7.62 (dd, J = 9.2, 1.8 Hz, 1H), 3.27-3.21 |
| | | (m, 2H), 2.93-2.85 (m, 1H), 2.81-2.72 (m, 1H), 1.63-1.52 (m, |
| | | 1H), 1.50-1.39 (m, 1H), 1.34-1.25 (m, 2H), 1.10-1.04 (m, 3H), |
| | | 0.86-0.77 (m, 3H); ESI MS (m/z): 343.1 [M + 1]. |
| 391 | ethyl(ethylimino)(2- | ¹H-NMR (400 MHz, DMSO-d₆) δ 9.46 (d, J = 1.0 Hz, 1H), 9.33 |
| | (pyridin-3-yl)-2H- | (d, J = 2.4 Hz, 1H), 8.70 (dd, J = 4.6, 1.5 Hz, 1H), 8.44-8.56 |
| | indazol-5-yl)-λ⁶- | (1H), 8.35 (t, J = 0.9 Hz, 1H), 7.93 (d, J = 9.0 Hz, 1H), 7.61 (dd, |
| | sulfanone | J = 9.3, 1.7 Hz, 2H), 3.26 (dd, J = 7.5, 5.7 Hz, 2H), 2.89 (s, 1H), |
| | | 2.80 (d, J = 7.3 Hz, 1H), 1.07 (td, J = 7.2, 5.4 Hz, 6H) |
| | | ESI MS (m/z): 315.05 [M + 1]. |
| 397 | (ethylimino)(2-(5- | ¹H-NMR (400 MHz, DMSO-d₆) δ 9.50 (s, 1H), 9.26 (s, 1H), |
| | fluoropyridin-3-yl)- | 8.75 (d, J = 2.4 Hz, 1H), 8.54 (dt, J = 9.7, 2.3 Hz, 1H), 8.32 (s, |
| | 2H-indazol-5- | 1H), 7.93 (d, J = 9.0 Hz, 1H), 7.59 (dd, J = 9.3, 1.7 Hz, 1H), |
| | yl)(isopropyl)-λ⁶- | 3.39 (t, J = 6.8 Hz, 1H), 2.93-2.86 (m, 1H), 2.83-2.77 (m, 1H), |
| | sulfanone | 1.26 (d, J = 6.8 Hz, 3H), 1.07 (dd, J = 13.0, 6.8 Hz, 6H) |
| | | ESI MS (m/z): 347 [M + 1]. |
| 400 | (ethylimino)(2-(5- | ¹H-NMR (400 MHz, DMSO-d₆) δ 9.52 (s, 1H), 9.29 (s, 1H), |
| | fluoropyridin-3-yl)- | 8.90 (d, J = 2.2 Hz, 1H), 8.79 (d, J = 2.2 Hz, 2H), 8.60 (dd, J = |
| | 2H-pyrazolo[3,4- | 9.8, 2.4 Hz, 1H), 3.50 (t, J = 6.7 Hz, 1H), 2.96-2.89 (m, 1H), |
| | b]pyridin-5- | 2.87-2.80 (m, 1H), 1.27 (d, J = 6.6 Hz, 3H), 1.13 (d, J = 6.8 Hz, |
| | yl)(isopropyl)-λ⁶- | 3H), 1.08 (t, J = 7.2 Hz, 3H); ESI MS (m/z): 348 [M + 1]. |
| | sulfanone | |
| 411 | (cyclopropylmethyl)(eth- | ¹H-NMR (400 MHz, DMSO-d₆) δ 9.50 (d, J = 0.9 Hz, 1H), 9.27 |
| | ylimino)(2-(5- | (s, 1H), 8.74 (d, J = 2.4 Hz, 1H), 8.54 (dt, J = 9.9, 2.4 Hz, 1H), |
| | fluoropyridin-3-yl)- | 8.38 (d, J = 0.6 Hz, 1H), 7.91 (d, J = 9.2 Hz, 1H), 7.66 (dd, J = |
| | 2H-indazol-5-yl)-λ⁶- | 9.2, 1.5 Hz, 1H), 3.24 (d, J = 7.3 Hz, 2H), 2.96-2.88 (m, 1H), |
| | sulfanone | 2.86-2.77 (m, 1H), 1.07 (t, J = 7.2 Hz, 3H), 0.91-0.85 (m, 1H), |
| | | 0.45-0.38 (m, 1H), 0.35-0.29 (m, 1H), 0.11 (td, J = 9.6, 4.9 Hz, |
| | | 1H), −0.05 (td, J = 9.9, 4.8 Hz, 1H); ESI MS (m/z): 358.95 |
| | | [M + 1]. |
| 419 | (ethylimino)(isoprop- | ¹H-NMR (400 MHz, DMSO-d₆) δ 9.54 (s, 2H), 9.51 (d, J = 0.9 |
| | yl)(2-(pyrimidin-5- | Hz, 1H), 9.31 (s, 1H), 8.34 (t, J = 0.8 Hz, 1H), 7.94 (d, J = 9.2 |
| | yl)-2H-indazol-5-yl)- | Hz, 1H), 7.60 (dd, J = 9.2, 1.8 Hz, 1H), 3.42-3.36 (m, 1H), 2.94- |
| | λ⁶-sulfanone | 2.86 (m, 1H), 2.83-2.75 (m, 1H), 1.26-1.21 (m, 3H), 1.10-1.03 |
| | | (m, 6H); ESI MS (m/z): 329.5 [M + 1]. |
| 457 | ethyl(ethylimino)(2- | ¹H-NMR (400 MHz, DMSO-d₆) δ 9.55 (s, 2H), 9.52 (d, J = 0.9 |
| | (pyrimidin-5-yl)-2H- | Hz, 1H), 9.31 (s, 1H), 8.38 (d, J = 0.6 Hz, 1H), 7.95 (d, J = 9.2 |
| | indazol-5-yl)-λ⁶- | Hz, 1H), 7.64 (dd, J = 9.2, 1.5 Hz, 1H), 3.30-3.23 (m, 2H), 2.92- |
| | sulfanone | 2.86 (m, 1H), 2.83-2.76 (m, 1H), 1.07 (dd, J = 13.3, 7.2 Hz, 6H) |
| | | ESI MS (m/z): 316 [M + 1]. |
| 461 | ethyl(ethylimino)(2- | ¹H-NMR (400 MHz, DMSO-d₆) δ 9.50 (d, J = 0.7 Hz, 1H), 9.26 |
| | (5-fluoropyridin-3- | (s, 1H), 8.74 (d, J = 2.4 Hz, 1H), 8.54 (dt, J = 9.9, 2.3 Hz, 1H), |
| | yl)-2H-indazol-5-yl)- | 8.35 (t, J = 0.7 Hz, 1H), 7.93 (d, J = 9.3 Hz, 1H), 7.62 (dd, J = |
| | λ⁶-sulfanone | 9.0, 1.7 Hz, 1H), 3.30-3.21 (m, 2H), 2.94-2.86 (m, 1H), 2.83- |
| | | 2.74 (m, 1H), 1.07 (q, J = 7.1 Hz, 6H); ESI MS (m/z): 332.95 |
| | | [M + 1]. |
| 466 | (cyclobutylmethyl)(eth- | ¹H-NMR (400 MHz, DMSO-d₆) δ 9.55 (s, 2H), 9.51 (s, 1H), |
| | ylimino)(2- | 9.31 (s, 1H), 8.37 (s, 1H), 7.93 (d, J = 9.2 Hz, 1H), 7.63 (dd, J = |
| | (pyrimidin-5-yl)-2H- | 9.2, 1.8 Hz, 1H), 3.42 (td, J = 14.0, 7.2 Hz, 2H), 2.90-2.83 (m, |
| | indazol-5-yl)-λ⁶- | 1H), 2.80-2.73 (m, 1H), 2.59 (d, J = 7.3 Hz, 1H), 1.95-1.92 (m, |
| | sulfanone | 1H), 1.73-1.64 (m, 4H), 1.56-1.51 (m, 1H), 1.05 (t, J = 7.2 Hz, |
| | | 3H); ESI MS (m/z): 356.05 [M + 1]. |
| 471 | (cyclobutylmethyl)(eth- | ¹H-NMR (400 MHz, CHLOROFORM-D) δ 9.05 (d, J = 1.8 Hz, |
| | ylimino)(2-(5- | 1H), 8.68 (d, J = 0.9 Hz, 1H), 8.59 (d, J = 2.4 Hz, 1H), 8.43 (d, |
| | fluoropyridin-3-yl)- | J = 0.6 Hz, 1H), 8.13 (dt, J = 9.0, 2.4 Hz, 1H), 7.89 (dd, J = 9.2, |
| | 2H-indazol-5-yl)-λ⁶- | 0.9 Hz, 1H), 7.68 (dd, J = 9.2, 1.5 Hz, 1H), 3.45-3.34 (m, 2H), |
| | sulfanone | 3.10-3.01 (m, 1H), 2.94-2.86 (m, 1H), 2.81-2.73 (m, 1H), 2.14- |
| | | 2.06 (m, 1H), 1.92-1.71 (m, 4H), 1.58-1.50 (m, 1H), 1.20 (t, J = |
| | | 7.3 Hz, 3H); ESI MS (m/z): 373.2 [M + 1]. |
| 474 | (ethylimino)(methyl) | ¹H-NMR (400 MHz, CHLOROFORM-D) δ 9.38 (d, J = 3.4 Hz, |
| | (2-(pyrimidin-5-yl)- | 2H), 9.33 (s, 1H), 8.71 (s, 1H), 8.54 (s, 1H), 7.96 (d, J = 9.2 Hz, |
| | 2H-indazol-5-yl)-λ⁶- | 1H), 7.77 (d, J = 9.2 Hz, 1H), 3.25 (s, 3H), 3.12-3.02 (m, 1H), |
| | sulfanone | 2.92 (dt, J = 19.8, 7.0 Hz, 1H), 1.22 (q, J = 6.7 Hz, 3H) |
| | | ESI MS (m/z): 302.15 [M + 1]. |
| 489 | (ethylimino)(2-(5- | ¹H-NMR (400 MHz, DMSO-d₆) δ 9.50 (d, J = 1.0 Hz, 1H), 9.26 |
| | fluoropyridin-3-yl)- | (s, 1H), 8.74 (d, J = 2.7 Hz, 1H), 8.54 (dt, J = 9.8, 2.3 Hz, 1H), |
| | 2H-indazol-5- | 8.36 (q, J = 0.8 Hz, 1H), 7.93 (d, J = 9.0 Hz, 1H), 7.63 (dd, J = |
| | yl)(propyl)-λ⁶- | 9.0, 1.7 Hz, 1H), 3.23 (t, J = 7.9 Hz, 2H), 2.93-2.85 (m, 1H), |
| | sulfanone | 2.81-2.73 (m, 1H), 1.66-1.46 (m, 2H), 1.05 (t, J = 7.1 Hz, 3H), |
| | | 0.87 (t, J = 7.5 Hz, 3H); ESI MS (m/z): 346.95 [M + 1]. |
| 490 | sec- | ¹H-NMR (400 MHz, DMSO-d₆) δ 9.49 (d, J = 0.7 Hz, 1H), 9.26 |
| | butyl(ethylimino)(2- | (s, 1H), 8.75 (d, J = 2.7 Hz, 1H), 8.54 (dt, J = 9.9, 2.4 Hz, 1H), |
| | (5-fluoropyridin-3- | 8.32 (s, 1H), 7.92 (dd, J = 9.0, 0.7 Hz, 1H), 7.59 (dd, J = 9.3, 1.7 |
| | yl)-2H-indazol-5-yl)- | Hz, 1H), 3.20-3.12 (m, 1H), 2.94-2.86 (m, 1H), 2.84-2.76 (m, |
| | λ⁶-sulfanone | 1H), 1.40-1.27 (m, 1H), 1.24 (d, J = 6.8 Hz, 2H), 1.09-1.05 (m, |
| | | 5H), 0.89 (dt, J = 32.4, 7.5 Hz, 3H); ESI MS (m/z): 361 [M + 1]. |
| 491 | (ethylimino)(3- | ¹H-NMR (400 MHz, DMSO-d₆) δ 9.51 (d, J = 1.0 Hz, 1H), 9.27 |
| | fluoropropyl)(2-(5- | (s, 1H), 8.75 (d, J = 2.2 Hz, 1H), 8.55 (dt, J = 9.9, 2.3 Hz, 1H), |
| | fluoropyridin-3-yl)- | 8.39 (d, J = 0.7 Hz, 1H), 7.94 (d, J = 9.0 Hz, 1H), 7.65 (dd, J = |
| | 2H-indazol-5-yl)-λ⁶- | 9.0, 1.7 Hz, 1H), 4.47 (dt, J = 47.1, 6.0 Hz, 2H), 3.38-3.33 (m, |
| | sulfanone | 2H), 2.95-2.76 (m, 2H), 2.04-1.84 (m, 2H), 1.07 (t, J = 7.2 Hz, |
| | | 3H); ESI MS (m/z): 364.9 [M + 1]. |
| 492 | (ethylimino)(2-(5- | ¹H-NMR (400 MHz, DMSO-d₆) δ 9.49 (s, 1H), 9.26 (s, 1H), |
| | fluoropyridin-3-yl)- | 8.75 (d, J = 2.4 Hz, 1H), 8.54 (dt, J = 9.9, 2.3 Hz, 1H), 8.33 (s, |
| | 2H-indazol-5- | 1H), 7.92 (d, J = 9.3 Hz, 1H), 7.60 (dd, J = 9.2, 1.6 Hz, 1H), |
| | yl)(pentan-3-yl)-λ⁶- | 3.06-3.00 (m, 1H), 2.89 (dt, J = 19.4, 7.2 Hz, 1H), 2.77 (dt, J = |
| | sulfanone | 19.4, 7.2 Hz, 1H), 1.98-1.90 (m, 1H), 1.79-1.68 (m, 2H), 1.54- |
| | | 1.46 (m, 1H), 1.07 (dd, J = 14.1, 6.7 Hz, 3H), 0.96 (t, J = 7.5 Hz, |
| | | 3H), 0.84 (t, J = 7.6 Hz, 3H); ESI MS (m/z): 375 [M + 1]. |
| 567 | cyclobutyl(ethylimi- | ¹H-NMR (400 MHz, DMSO-d₆) δ 9.45 (dd, J = 7.0, 0.9 Hz, 1H), |
| | no)(2-(pyridin-3-yl)- | 9.33 (q, J = 2.4 Hz, 1H), 8.70 (dd, J = 4.7, 1.4 Hz, 1H), 8.52- |
| | 2H-indazol-5-yl)-λ⁶- | 8.48 (m, 1H), 8.33 (q, J = 0.8 Hz, 1H), 7.91 (d, J = 8.9 Hz, 1H), |
| | sulfanone | 7.69-7.65 (m, 1H), 7.57 (dd, J = 9.2, 1.5 Hz, 1H), 4.13-4.05 (m, |
| | | 1H), 2.94-2.85 (m, 1H), 2.84-2.74 (m, 1H), 2.23-2.15 (m, 2H), |
| | | 1.91-1.75 (m, 3H), 1.07-1.03 (m, 4H); ESI MS (m/z): 340.95 |
| | | [M + 1]. |
| 568 | (cyclohexylmethyl)(eth- | ¹H-NMR (400 MHz, DMSO-d₆) δ 9.44 (d, J = 0.6 Hz, 1H), 9.33 |
| | ylimino)(2- | (d, J = 2.4 Hz, 1H), 8.70 (dd, J = 4.7, 1.4 Hz, 1H), 8.50 (dq, J = |
| | (pyridin-3-yl)-2H- | 8.3, 1.4 Hz, 1H), 8.37 (d, J = 0.9 Hz, 1H), 7.92 (d, J = 9.2 Hz, |
| | indazol-5-yl)-λ⁶- | 1H), 7.69-7.62 (m, 2H), 3.17 (dd, J = 6.0, 1.7 Hz, 2H), 2.85 (dd, |
| | sulfanone | J = 12.2, 7.3 Hz, 1H), 2.72 (dd, J = 12.2, 7.0 Hz, 1H), 1.84 (d, J = |
| | | 3.1 Hz, 2H), 1.56 (d, J = 43.4 Hz, 4H), 1.09-1.02 (m, 8H) |
| | | ESI MS (m/z): 383.05 [M + 1]. |
| 585 | (ethylimino)(pentan- | ¹H-NMR (400 MHz, DMSO-d₆) δ 9.44 (d, J = 0.7 Hz, 1H), 9.32 |
| | 3-yl)(2-(pyridin-3- | (d, J = 2.4 Hz, 1H), 8.70 (dd, J = 4.6, 1.5 Hz, 1H), 8.49 (dq, J = |
| | yl)-2H-indazol-5-yl)- | 8.3, 1.3 Hz, 1H), 8.32 (d, J = 0.7 Hz, 1H), 7.92 (d, J = 9.3 Hz, |
| | λ⁶-sulfanone | 1H), 7.68 (dd, J = 8.3, 4.9 Hz, 1H), 7.59 (dd, J = 9.2, 1.6 Hz, 1H), |
| | | 3.05-2.99 (m, 1H), 2.93-2.85 (m, 1H), 2.81-2.73 (m, 1H), 1.94 |
| | | (qd, J = 7.3, 4.6 Hz, 1H), 1.79-1.66 (m, 2H), 1.55-1.44 (m, 1H), |
| | | 1.10-1.02 (m, 3H), 0.96 (t, J = 7.5 Hz, 3H), 0.84 (t, J = 7.6 Hz, |
| | | 3H); ESI MS (m/z): 357.05 [M + 1]. |
| 586 | cyclopentyl(ethylimi- | ¹H-NMR (400 MHz, DMSO-d₆) δ 9.44 (s, 1H), 9.33 (d, J = 2.1 |
| | no)(2-(pyridin-3-yl)- | Hz, 1H), 8.70 (d, J = 4.0 Hz, 1H), 8.50 (d, J = 8.6 Hz, 1H), 8.34 |
| | 2H-indazol-5-yl)-λ⁶- | (s, 1H), 7.92 (d, J = 9.2 Hz, 1H), 7.68 (dd, J = 8.3, 4.9 Hz, 1H), |
| | sulfanone | 7.61 (d, J = 9.2 Hz, 1H), 3.77 (t, J = 7.5 Hz, 1H), 2.91-2.84 (m, |
| | | 1H), 2.76 (dt, J = 19.4, 7.1 Hz, 1H), 2.11 (dd, J = 13.4, 7.6 Hz, |
| | | 1H), 1.94 (d, J = 5.5 Hz, 1H), 1.73 (dd, J = 12.2, 7.0 Hz, 1H), 1.54 |
| | | (dd, J = 15.3, 6.7 Hz, 4H), 1.48 (t, J = 6.6 Hz, 1H), 1.04 (t, J = 7.2 |
| | | Hz, 3H); ESI MS (m/z): 354.95 [M + 1]. |
| 587 | cycloheptyl(ethylimi- | ¹H-NMR (400 MHz, DMSO-d₆) δ 9.44 (d, J = 0.6 Hz, 1H), 9.32 |
| | no)(2-(pyridin-3-yl)- | (d, J = 2.4 Hz, 1H), 8.70 (dd, J = 4.7, 1.4 Hz, 1H), 8.49 (dq, J = |
| | 2H-indazol-5-yl)-λ⁶- | 8.3, 1.4 Hz, 1H), 8.31 (d, J = 0.6 Hz, 1H), 7.91 (d, J = 9.2 Hz, |
| | sulfanone | 1H), 7.70-7.66 (m, 1H), 7.58 (dd, J = 9.0, 1.7 Hz, 1H), 3.28-3.23 |
| | | (m, 1H), 2.94-2.85 (m, 1H), 2.82-2.74 (m, 1H), 2.30-2.23 (m, |
| | | 1H), 1.97-1.91 (m, 1H), 1.68-1.59 (m, 3H), 1.54-1.31 (m, 7H), |
| | | 1.05 (q, J = 7.2 Hz, 3H); ESI MS (m/z): 382.95 [M + 1]. |
| 588 | (2- | ¹H-NMR (400 MHz, DMSO-d₆) δ 9.45 (d, J = 0.9 Hz, 1H), 9.33 |
| | cyclohexylethyl)(eth- | (d, J = 2.4 Hz, 1H), 8.70 (dd, J = 4.7, 1.4 Hz, 1H), 8.50 (dq, J = |
| | ylimino)(2-(pyridin- | 8.3, 1.3 Hz, 1H), 8.35 (d, J = 0.6 Hz, 1H), 7.92 (d, J = 9.2 Hz, |
| | 3-yl)-2H-indazol-5- | 1H), 7.68 (dd, J = 8.3, 4.9 Hz, 1H), 7.62 (dd, J = 9.2, 1.8 Hz, 1H), |
| | yl)-λ⁶-sulfanone | 3.28-3.23 (m, 2H), 2.91-2.84 (m, 1H), 2.81-2.74 (m, 1H), 1.59- |
| | | 1.47 (m, 6H), 1.36-1.30 (m, 1H), 1.24-1.20 (m, 1H), 1.11-1.01 |
| | | (m, 6H), 0.77 (t, J = 10.5 Hz, 2H); ESI MS (m/z): 397 [M + 1]. |
| 589 | (ethylimino)(isobu- | ¹H-NMR (400 MHz, DMSO-d₆) δ 9.45 (s, 1H), 9.33 (d, J = 2.1 |
| | tyl)(2-(pyridin-3-yl)- | Hz, 1H), 8.70 (d, J = 3.7 Hz, 1H), 8.51-8.49 (m, 1H), 8.37 (s, 1H), |
| | 2H-indazol-5-yl)-λ⁶- | 7.93 (d, J = 9.2 Hz, 1H), 7.68 (dd, J = 8.3, 4.9 Hz, 1H), 7.63 (dd, |
| | sulfanone | J = 9.2, 1.5 Hz, 1H), 3.23-3.12 (m, 2H), 2.89-2.82 (m, 1H), 2.78- |
| | | 2.71 (m, 1H), 2.11-2.05 (m, 1H), 1.04 (t, J = 7.2 Hz, 3H), 0.98 (d, |
| | | J = 6.7 Hz, 3H), 0.87 (d, J = 6.7 Hz, 3H) |
| | | ESI MS (m/z): 343 [M + 1]. |
| 595 | cyclohexyl(ethylimi- | ¹H-NMR (400 MHz, DMSO-d₆) δ 9.48 (s, 1H), 9.33 (d, J = 2.7 |
| | no)(2-(pyridin-3-yl)- | Hz, 1H), 8.71 (dd, J = 4.6, 1.5 Hz, 1H), 8.51 (d, J = 9.3 Hz, 1H), |
| | 2H-indazol-5-yl)-λ⁶- | 8.44 (s, 1H), 7.97 (d, J = 8.6 Hz, 1H), 7.69 (dd, J = 8.2, 4.8 Hz, |
| | sulfanone | 1H), 7.61 (dd, J = 9.3, 1.2 Hz, 1H), 3.62-3.58 (m, 1H), 2.00 (d, |
| | | J = 6.6 Hz, 3H), 1.35-1.11 (m, 6H), 0.83 (dt, J = 36.4, 7.2 Hz, 3H) |
| | | ESI MS (m/z): 369 [M + 1]. |
| 597 | (ethylimino)(2- | ¹H-NMR (400 MHz, DMSO-d₆) δ 9.45 (dd, J = 6.1, 0.9 Hz, 1H), |
| | (pyridin-3-yl)-2H- | 9.33 (d, J = 2.8 Hz, 1H), 8.71-8.69 (m, 1H), 8.52-8.48 (m, 1H), |
| | indazol-5- | 8.38-8.35 (m, 1H), 7.90 (q, J = 4.6 Hz, 1H), 7.69-7.62 (m, 2H), |
| | yl)((tetrahydrofuran- | 4.14 (td, J = 6.2, 2.5 Hz, 1H), 3.54-3.40 (m, 5H), 2.93-2.73 (m, |
| | 2-yl)methyl)-λ⁶- | 2H), 1.77-1.68 (m, 2H), 1.63-1.48 (m, 1H), 1.09-1.03 (m, 3H) |
| | sulfanone | ESI MS (m/z): 370.95 [M + 1]. |
| 603 | 4-(N-ethyl-2- | ¹H-NMR (400 MHz, MeOD) δ 9.32 (d, J = 2.2 Hz, 1H), 9.25 (d, |
| | (pyridin-3-yl)-2H- | J = 1.0 Hz, 1H), 8.69 (dd, J = 4.8, 1.3 Hz, 1H), 8.55-8.52 (m, 2H), |
| | indazole-5- | 7.97-7.95 (m, 1H), 7.72-7.68 (m, 2H), 3.46-3.42 (m, 2H), 3.08- |
| | sulfonimidoyl)bu- | 3.00 (m, 1H), 2.97-2.89 (m, 1H), 2.62 (t, J = 7.2 Hz, 2H), 2.13- |
| | tanenitrile | 1.95 (m, 2H), 1.19 (t, J = 7.2 Hz, 3H) |
| | | ESI MS (m/z): 353.9 [M + 1]. |
| 604 | (ethylimino)(iso- | ¹H-NMR (400 MHz, CHLOROFORM-D) δ 9.22 (d, J = 2.4 Hz, |
| | pentyl)(2-(pyridin-3- | 1H), 8.74-8.73 (m, 1H), 8.69 (d, J = 0.7 Hz, 1H), 8.50 (s, 1H), |
| | yl)-2H-indazol-5-yl)-λ⁶- | 8.30 (dq, J = 8.3, 1.4 Hz, 1H), 7.95-7.90 (m, 1H), 7.71 (dd, J = |
| | sulfanone | 9.3, 1.7 Hz, 1H), 7.56-7.52 (m, 1H), 3.49-3.27 (m, 2H), 3.13-3.05 |
| | | (m, 1H), 2.99-2.90 (m, 1H), 1.78-1.68 (m, 1H), 1.67-1.57 (m, |
| | | 1H), 1.50-1.41 (m, 1H), 1.25-1.21 (m, 3H), 0.86 (dd, J = 8.7, 6.5 |
| | | Hz, 6H); ESI MS (m/z): 357.05 [M + 1]. |
| 612 | (ethylimino)(2- | ¹H-NMR (400 MHz, METHANOL-D4) δ 9.31 (d, J = 2.4 Hz, |
| | (pyridin-3-yl)-2H- | 1H), 9.24 (s, 1H), 8.68 (dd, J = 4.6, 1.2 Hz, 1H), 8.54-8.51 (m, |
| | indazol-5-yl)(3,3,3- | 2H), 7.96 (d, J = 9.2 Hz, 1H), 7.73-7.67 (m, 2H), 3.59-3.48 (m, |
| | trifluoropropyl)-λ⁶- | 2H), 3.07-2.92 (m, 2H), 2.76-2.48 (m, 2H), 1.19 (t, J = 7.3 Hz, |
| | sulfanone | 3H); ESI MS (m/z): 382.9 [M + 1]. |
| 615 | (ethylimino)(pentan- | H-NMR (400 MHz, CHLOROFORM-D) δ 9.22 (d, J = 2.4 Hz, |
| | 2-yl)(2-(pyridin-3- | 1H), 8.73 (dd, J = 4.7, 1.4 Hz, 1H), 8.65 (t, J = 1.2 Hz, 1H), 8.39 |
| | yl)-2H-indazol-5-yl)- | (s, 1H), 8.32-8.29 (m, 1H), 7.90 (dd, J = 9.2, 1.2 Hz, 1H), 7.66 |
| | λ⁶-sulfanone | (dt, J = 9.2, 1.4 Hz, 1H), 7.54 (dd, J = 8.3, 4.9 Hz, 1H), 3.19-3.05 |
| | | (m, 2H), 3.00-2.91 (m, 1H), 2.24-1.83 (m, 1H), 1.43-1.36 (m, |
| | | 3H), 1.28-1.18 (m, 6H), 0.95-0.83 (m, 3H) |
| | | ESI MS (m/z): 357.15 [M + 1]. |
| 617 | (ethylimino)(propyl) | ¹H-NMR (400 MHz, DMSO-d₆) δ 9.46 (d, J = 1.0 Hz, 1H), 9.33 |
| | (2-(pyridin-3-yl)-2H- | (d, J = 2.4 Hz, 1H), 8.70 (dd, J = 4.6, 1.5 Hz, 1H), 8.50 (dq, J = |
| | indazol-5-yl)-λ⁶- | 8.3, 1.4 Hz, 1H), 8.35 (q, J = 0.8 Hz, 1H), 7.94-7.91 (m, 1H), 7.68 |
| | sulfanone | (ddd, J = 8.4, 4.7, 0.7 Hz, 1H), 7.62 (dd, J = 9.3, 1.7 Hz, 1H), |
| | | 3.27-3.18 (m, 2H), 2.93-2.84 (m, 1H), 2.81-2.73 (m, 1H), 1.66- |
| | | 1.45 (m, 2H), 1.06 (t, J = 7.1 Hz, 3H), 0.87 (t, J = 7.5 Hz, 3H) |
| | | ESI MS (m/z): 329.05 [M + 1]. |
| 618 | (ethylimino)(neo- | ¹H-NMR (400 MHz, DMSO-d₆) δ 9.59 (s, 1H), 9.36 (d, J = 2.2 |
| | pentyl)(2-(pyridin-3- | Hz, 1H), 8.79 (s, 1H), 8.74 (dd, J = 4.8, 1.3 Hz, 1H), 8.53 (dq, J = |
| | yl)-2H-indazol-5-yl)-λ⁶- | 8.3, 1.4 Hz, 1H), 8.14 (d, J = 9.3 Hz, 1H), 7.82 (dd, J = 9.3, 2.0 |
| | sulfanone | Hz, 1H), 7.71 (dd, J = 8.1, 4.4 Hz, 1H), 4.13 (d, J = 30.6 Hz, 2H), |
| | | 3.10-2.94 (m, 2H), 1.11 (t, J = 7.2 Hz, 3H), 1.03 (d, J = 14.9 Hz, |
| | | 9H); ESI MS (m/z): 357.05 [M + 1]. |

### Example 14: Synthesis of N-(isopropyl(oxo)(2-(pyridin-3-yl)-2H-indazol-5-yl)-λ⁶-sulfaneylidene) cyanamide 392

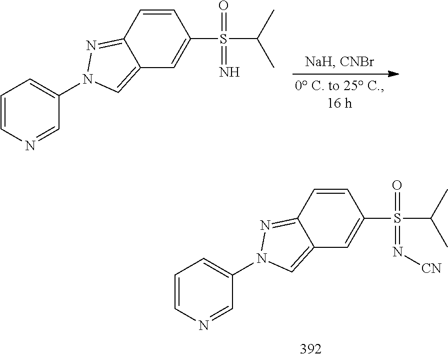 [SMILES: CC(C)S(=N)(=O)c1ccc2nn(-c3cccnc3)cc2c1 ; CC(C)S(=O)(=NC#N)c1ccc2nn(-c3cccnc3)cc2c1]

To a stirred solution of imino(isopropyl)(2-(pyridin-3-yl)-2H-indazol-5-yl)-λ⁶-sulfanone (300 mg, 0.999 mmol) in N,N-dimethylformamide (5 mL), sodium hydride (59.9 mg, 1.498 mmol) was charged under nitrogen atmosphere, at 25° C., and stirring was continued for 5-10 another minutes. Afterwards, the reaction mixture was cooled to 0° C. and cyanogen bromide (159 mg, 1.498 mmol) was added at this temperature under continued stirring. After completion of the addition, the reaction mixture was allowed to warm at 25° C. and stirred for 12 h at this temperature. After completion of the reaction, the mixture was poured into ice-cold water and the aqueous layer was extracted with ethyl acetate (3×25 mL). The organic layer was separated, washed with brine solution (2×25 mL), dried over anhydrous sodium sulfate, filtered and concentrated under reduced pressure to obtain a crude oil. The crude material was purified by flash chromatography to obtain the desired N-(isopropyl(oxo)(2-(pyridin-3-yl)-2H-indazol-5-yl)-λ⁶-sulfaneylidene)cyanamide 392 (130 mg, 0.400 mmol, 40.0% yield).

TABLE 14
Representative compound of the present disclosure were prepared according to
the suitable starting material and methods as described in the Example 14.

| Compd | | |
|---|---|---|
| no. | Compound name | Analytical Data |
| 392 | N-(isopropyl(oxo)(2- | ¹H-NMR (400 MHz, DMSO-d₆) δ 9.59 (d, J = 0.7 Hz, 1H), 9.35 |
| | (pyridin-3-yl)-2H- | (d, J = 2.4 Hz, 1H), 8.74 (dd, J = 4.6, 1.5 Hz, 1H), 8.66 (d, J = |
| | indazol-5-yl)-λ⁶- | 1.2 Hz, 1H), 8.53 (dq, J = 8.3, 1.4 Hz, 1H), 8.10 (d, J = 9.3 Hz, |
| | sulfaneylidene)cyanamide | 1H), 7.75-7.69 (m, 2H), 1.37 (d, J = 6.8 Hz, 3H), 1.23 (d, J = 6.6 |
| | | Hz, 4H); ESI MS (m/z): 325.5 [M + 1]. |

### Example 15: Synthesis of ((2,2-difluoroethyl)imino)(isopropyl)(2-(pyridin-3-yl)-2H-indazol-4-yl)-λ⁶-sulfanone 424

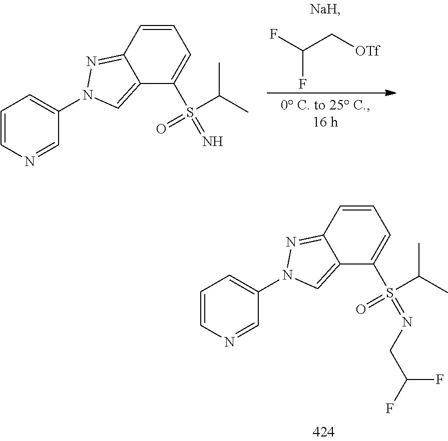 [SMILES: CC(C)S(=N)(=O)c1cccc2nn(-c3cccnc3)cc12 ; O=S(=O)(OCC(F)F)C(F)(F)F ; CC(C)S(=O)(=NCC(F)F)c1cccc2nn(-c3cccnc3)cc12]

To a stirred solution of imino(isopropyl)(2-(pyridin-3-yl)-2H-indazol-4-yl)-λ⁶-sulfanone (300 mg, 0.999 mmol) in N,N-dimethylformamide (5 mL) sodium hydride (59.9 mg, 1.498 mmol) was charged under nitrogen atmosphere, at 25° C., and stirring was continued for another 5-10 minutes. Afterwards, the reaction mixture was cooled to 0° C. and 2,2-difluoroethyl trifluoromethanesulfonate (662 μl, 4.99 mmol) was added at this temperature under stirring. After completion of the addition, the reaction mixture was allowed to warm at 25° C. and stirred for 16 h at this temperature. After completion of the reaction, the reaction mixture was poured into ice-cold water and the aqueous layer was extracted with ethyl acetate (3×25 mL). The organic layer was separated, washed with brine solution (2×25 mL), dried over anhydrous sodium sulfate, filtered and concentrated under reduced pressure to obtain a crude oil. The crude compound was purified by flash chromatography to get the desired ((2,2-difluoroethyl)imino)(isopropyl)(2-(pyridin-3-yl)-2H-indazol-4-yl)-λ⁶-sulfanone 424 (70 mg, 0.192 mmol, 19.23% yield).

TABLE 15
Representative compound of the present disclosure were prepared according to
the suitable starting material and methods as described in the Example 15.

| Compd | | |
|---|---|---|
| no. | Compound name | Analytical Data |
| 424 | ((2,2- | ¹H-NMR (400 MHz, DMSO-d₆) δ 9.36 (d, J = 2.4 Hz, 1H), 9.23 |
| | difluoroethyl)imi- | (d, J = 0.5 Hz, 1H), 8.70 (dd, J = 4.6, 1.2 Hz, 1H), 8.55 (dq, J = |
| | no)(isopropyl)(2- | 8.3, 1.3 Hz, 1H), 8.13-8.08 (m, 1H), 7.69-7.63 (m, 2H), 7.58 |
| | (pyridin-3-yl)-2H- | (dd, J = 8.6, 7.1 Hz, 1H), 6.01 (tt, J = 56.3, 4.2 Hz, 1H), 3.73- |
| | indazol-4-yl)-λ⁶- | 3.66 (m, 1H), 3.25-3.00 (m, 2H), 1.36-1.28 (m, 3H), 1.07 (d, J = |
| | sulfanone | 6.6 Hz, 3H); ESI MS (m/z): 364.95 [M + 1]. |

* Compound names were generated using Chemdraw Professional 19.1

The compounds of formula A-1, A-2 or A-3 are synthesized according to the general scheme 6-14 as described in the description. Particularly, the compounds of formula A-1, wherein A₁, A₂ and A₃ represent C are synthesized according to the procedure described in WO 201614435 and the compounds of formula A-1, wherein one of the A₁, A₂ and A₃ represent N are synthesized according to the procedure as described below.

### Reaction Scheme

### Example-16

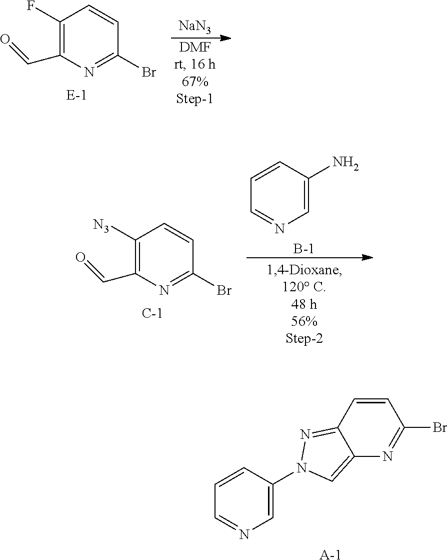

### 3-azido-6-bromopicolinaldehyde

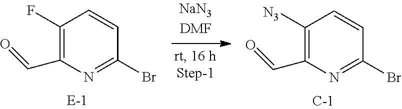

To a stirred solution of 6-bromo-3-fluoropicolinaldehyde E-1 (8 g, 39.2 mmol) in N,N-dimethylformamide (80 ml), sodium azide (3.06 g, 47.1 mmol) was added and the resulting mixture was stirred at 25° C. for 16 h. After completion of the reaction, the reaction mixture was diluted with water and the aqueous layer was extracted with ethyl acetate (3×100 ml). The organic layer was separated and washed with a brine solution (1×100 ml). The organic layer was dried over anhydrous sodium sulfate, filtered and concentrated under reduced pressure to obtain 3-azido-6-bromopicolinaldehyde C-1 (6 g, 26.4 mmol, 67.4% yield). The TLC was compared with the authentic spot.

### 5-bromo-2-(pyridin-3-yl)-2H-pyrazolo[4,3-b]pyridine

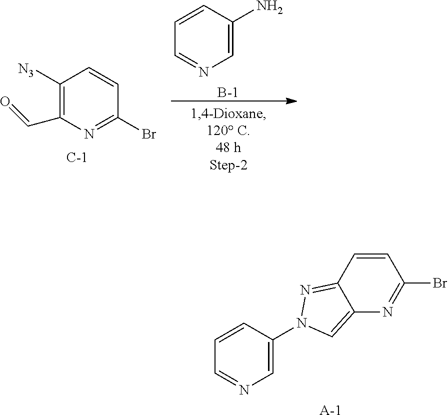

To a stirred solution of 3-azido-6-bromopicolinaldehyde C-1 (6 g, 26.4 mmol) in 1,4-dioxane (60 ml), pyridin-3-amine B-1 (2.74 g, 29.1 mmol) was added and the resulting mixture was stirred at 120° C. for 48 h. After completion of the reaction, the reaction mixture was concentrated under reduced pressure and water was added to obtain a crude solid which was filtered, collected and further purified by flash chromatography to get the desired 5-bromo-2-(pyridin-3-yl)-2H-pyrazolo[4,3-b]pyridine A-1 (4.1 g, 14.90 mmol, 56.4% yield).

As described herein, the compounds of formula (I) show insecticidal activities which are exerted with respect to numerous insects attacking on important agricultural crops. The compounds of the present invention were assessed for their activity as described in the following tests:

### BIOLOGY EXAMPLES

### Example A: Bemisia tabaci

The leaf dip method was used for testing, wherein the required quantity of the compound was weighed and dissolved in a tube containing a solvent solution. The tube was put on a vortex at 2000 rpm for proper mixing and then diluted with a 0.01% Triton-X solution to the desired test concentration. Brinjal leaves were dipped in the compound solution for 10 seconds; shade dried for 20 min and then placed, with the abaxial side of the leaf up, on 4 ml of a solidified 1% agar-agar solution in respective container caps. Known numbers of freshly emerged whitefly adults were collected, using a modified aspirator, and released into a perforated container in which the cap containing the treated leaf was placed. The containers were kept in a plant growth chamber at a temperature of 25° C. and relative humidity of 70%. Observations on dead, moribund and alive adults were recorded 72 h after the release. Percent mortality was calculated by combining dead and moribund adults and comparing the result to the one of the untreated controls. The compounds 1 9 11 18 25 28 29 30 31

| | | | | | | | | | | | |
|---|---|---|---|---|---|---|---|---|---|---|---|
| | | | 1 | 9 | 11 | 18 | 25 | 28 | 29 | 30 | 31 |
| 32 | 33 | 34 | 38 | 43 | 48 | 65 | 66 | 68 | 71 | 72 | 73 |
| 74 | 77 | 78 | 79 | 86 | 87 | 93 | 101 | 104 | 108 | 109 | 110 |
| 116 | 119 | 122 | 123 | 124 | 126 | 135 | 184 | 186 | 193 | 213 | 214 |
| 215 | 216 | 218 | 219 | 220 | 221 | 222 | 223 | 224 | 226 | 227 | 228 |
| 231 | 236 | 237 | 238 | 239 | 240 | 241 | 248 | 249 | 250 | 254 | 255 |
| 278 | 287 | 288 | 289 | 290 | 291 | 292 | 293 | 294 | 296 | 306 | 307 |
| 308 | 309 | 310 | 311 | 312 | 313 | 314 | 337 | 354 | 355 | 358 | 368 |
| 369 | 383 | 385 | 386 | 387 | 389 | 391 | 397 | 406 | 408 | 410 | 418 |
| 419 | 421 | 446 | 447 | 450 | 455 | 457 | 458 | 461 | 462 | 463 | 465 |
| 466 | 467 | 470 | 474 | 475 | 478 | 479 | 480 | 482 | 554 | 557 | 561 |

and 580 recorded ≥70 percent mortality @ 300 ppm.

### Example B: Myzus persicae

The leaf dip method was used for testing, wherein the required quantity of the compound was weighed and dissolved in a tube containing a solvent solution. The tube was put on a vortex at 2000 rpm for proper mixing and then diluted with a 0.01% Triton-X solution to the desired test concentration. Capsicum leaves were dipped in the compound solution for 10 seconds, shade dried for 20 min and then placed, with the abaxial side of the leaf up, in single cells of a bio-assay tray containing 4 ml of a solidified 1% agar-agar solution. Known numbers of third instar nymphs of Myzus persicae, collected in petri plates, were released into the cell with the treated leaf and the cell was covered with a perforated lid for better aeration. The trays were kept in a plant growth chamber at a temperature of 25° C. and a relative humidity of 70%. Observations on dead, moribund and alive nymphs were recorded 72 h after the release. Percent mortality was calculated by combining dead and moribund nymphs and comparing the result with the one of the untreated controls. The compounds

| | | | | | | | | | | | |
|---|---|---|---|---|---|---|---|---|---|---|---|
| | | | | | | | 1 | 2 | 3 | 25 | 28 |
| 29 | 32 | 41 | 64 | 70 | 71 | 73 | 88 | 89 | 90 | 92 | 99 |
| 102 | 110 | 112 | 119 | 123 | 124 | 136 | 137 | 138 | 146 | 147 | 148 |
| 156 | 168 | 180 | 193 | 216 | 217 | 218 | 224 | 236 | 237 | 238 | 239 |
| 240 | 248 | 249 | 250 | 254 | 255 | 256 | 287 | 288 | 290 | 291 | 306 |
| 312 | 314 | 337 | 354 | 383 | 386 | 387 | 388 | 389 | 390 | 397 | 406 |
| 419 | 455 | 456 | 457 | 458 | 460 | 461 | 478 | 479 | 480 | 482 | 483 |
| 484 | 485 | 486 | 487 | 488 | 490 | 491 | 492 | 557 | 558 | 560 | 564 |

| | |
|---|---|
| 567 | 569 and 581 recorded ≥70 percent mortality @ 300 ppm. |

### Example C: Nilaparvata lugens

The seedling dip method was used for testing, wherein the required quantity of the compound was weighed and dissolved in a tube containing a solvent solution. The tube was put on a vortex at 2000 rpm for proper mixing and then diluted with a 0.01% Triton-X solution to the desired test concentration. Paddy seedlings were dipped in the compound solution for 10 seconds, shade dried for 20 min and then the seedlings were placed in glass test tubes with the roots kept in water. 15 third instar nymphs of Nilaparvata lugens were released into each test tube and the tubes were kept in a plant growth chamber at a temperature of 25° C. and relative humidity of 75%. Observations on dead, moribund and alive nymphs were recorded 72 h after the release. Percent mortality was calculated by combining dead and moribund nymphs and comparing the result to the one of the untreated controls. The compounds

| | | | | | | | | | | | |
|---|---|---|---|---|---|---|---|---|---|---|---|
| | | | | | | | | | | | 1 |
| 2 | 3 | 4 | 5 | 7 | 8 | 9 | 11 | 12 | 13 | 14 | 16 |
| 19 | 20 | 21 | 23 | 26 | 27 | 28 | 29 | 30 | 32 | 33 | 38 |
| 41 | 42 | 51 | 64 | 71 | 72 | 73 | 78 | 86 | 87 | 92 | 93 |
| 104 | 105 | 111 | 112 | 113 | 114 | 115 | 125 | 126 | 127 | 137 | 146 |
| 147 | 148 | 152 | 153 | 155 | 181 | 193 | 194 | 202 | 206 | 236 | 238 |
| 240 | 331 | 337 | 343 | 347 | 354 | 355 | 356 | 357 | 358 | 359 | 360 |
| 367 | 385 | 386 | 387 | 388 | 389 | 391 | 392 | 394 | 396 | 397 | 402 |
| 406 | 417 | 419 | 446 | 447 | 450 | 454 | 457 | 461 | 462 | 464 | 470 |

| | |
|---|---|
| 471 | 474 and 475 recorded ≥70 percent mortality @ 300 ppm. |

Having described the invention with reference to certain preferred embodiments, other embodiments will become apparent to one skilled in the art from consideration of the specification.

1. A compound of formula (I), 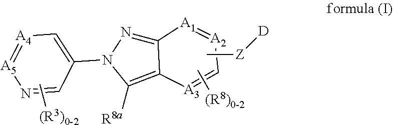
wherein,
Z is selected from a direct bond or —C(═O)—;
D is selected from the group consisting of D₁, D₂ and D₃, 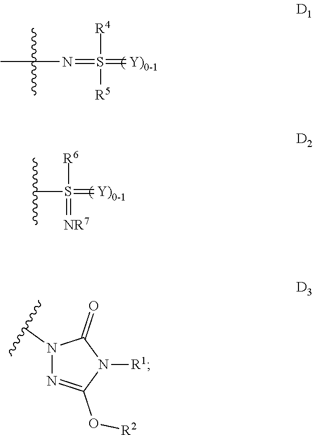
Y represents O or NR⁷;
A₁, A₂, and A₃ are independently C or N;
A₄ and A₅ are independently C or N, provided that both A₄ and A₅ simultaneously cannot be N;
R¹ is selected from the group consisting of C₁-C₆ alkyl, C₂-C₆ alkenyl, C₂-C₆ alkynyl, C₁-C₆ haloalkyl, C₃-C₈ cycloalkyl, C₃-C₈ cycloalkyl-C₁-C₆ alkyl, phenyl, benzyl, phenyl ethyl and C₂-C₆ heterocyclyl;
R² is selected from the group consisting of C₁-C₆ alkyl, C₂-C₆ alkenyl, C₂-C₆ alkynyl, C₁-C₆ haloalkyl, and C₃-C₈ cycloalkyl-C₁-C₆ alkyl;
R³ is selected from the group consisting of hydrogen, halogen, cyano, C₁-C₆ alkyl, C₂-C₆alkenyl, C₂-C₆ alkynyl, C₃-C₆ cycloalkyl, C₁-C₆ alkoxy and C₁-C₆ haloalkyl;
R⁴ is selected from the group consisting of C₁-C₆ alkyl, C₂-C₆ alkenyl, C₂-C₆ alkynyl, C₁-C₆ haloalkyl, C₂-C₆ haloalkenyl, C₃-C₈ cycloalkyl, C₃-C₈ cycloalkyl-C₁-C₆ alkyl and —NR^{c}R^{d};
R^{c} is selected from the group consisting of hydrogen, C₁-C₆ alkyl, C₁-C₆ haloalkyl, and C₃-C₈ cycloalkyl;
R^{d} is selected from the group consisting of hydrogen, C₁-C₆ alkyl, C₂-C₆ alkenyl, C₂-C₆ alkynyl, C₁-C₆ haloalkyl, C₂-C₆ haloalkenyl, C₃-C₈ cycloalkyl, C₃-C₆ cycloalkyl-C₁-C₄ alkyl;
or
R^{c} and R^{d} substituents together with the atom to which they are attached or together with further atoms selected from the group consisting of C, N, O, C(═O), C(═S), and S(O)₀₋₂ may form a 3- to 6-membered ring, which may optionally be substituted by one or more substituents selected from the group consisting of halogen, CN and C₁-C₆ alkyl;
R⁵ is selected from the group consisting of C₁-C₆ alkyl, C₁-C₆ haloalkyl, C₃-C₈ cycloalkyl, C₃-C₈ cycloalkyl-C₁-C₆ alkyl, C₂-C₆ alkenyl, C₂-C₆ haloalkenyl, phenyl, benzyl, and C₂-C₆ heterocyclyl; or
R⁴ and R⁵ substituents together with the atom to which they are attached or together with further atoms selected from the group consisting of C, N, O, C(═O), C(═S), and S(O)₀₋₂ may form a 3- to 6-membered ring, which may optionally be substituted by one or more substituents selected from the group consisting of halogen, CN and C₁-C₆ alkyl;
R⁶ is selected from the group consisting of C₁-C₆ alkyl, C₂-C₆ alkenyl, C₂-C₆ alkynyl, C₁-C₆ haloalkyl, C₂-C₆ haloalkenyl, C₃-C₈ cycloalkyl, C₃-C₈ cycloalkyl-C₁-C₆ alkyl, phenyl, benzyl, C₂-C₆ heterocyclyl, and —NR^{c}R^{d};
R⁷ is selected from the group consisting of hydrogen, cyano, C₁-C₆ alkyl, C₂-C₆ alkenyl, C₂-C₆ alkynyl, C₁-C₆ haloalkyl, C₂-C₆ haloalkenyl, C₁-C₆-alkoxy, C₃-C₈ cycloalkyl, C₃-C₈ cycloalkyl-C₁-C₆ alkyl, phenyl, benzyl, C₂-C₆ heterocyclyl, C₂-C₆ heterocyclyl-C₁-C₆ alkyl, —COR⁵, —CONR^{c}R^{d}, SCF₃, and —SO₂R⁵;
R⁸ and R^{8a} are independently selected from the group consisting of hydrogen, halogen, cyano, C₁-C₄-alkyl, C₁-C₄-haloalkyl;
each aliphatic group of R¹, R², R³, R⁴, R⁵, R⁶ and R⁷ may be optionally substituted with one or more groups of R^{a}; and cyclic groups of R¹, R², R³, R⁴, R⁵, R⁶ and R⁷ may optionally be substituted with one or more groups of R^{b}, wherein,
R^{a} is selected from the group consisting of halogen, cyano, C₁-C₆ alkyl, C₁-C₆ haloalkyl, C₃-C₈ cycloalkyl, C₁-C₆ alkoxy, C₁-C₆ alkylthio, phenyl and C₂-C₆ heterocyclyl;
R^{b} is selected from the group consisting of halogen, cyano, C₁-C₆ alkyl, C₂-C₆ alkenyl, C₂-C₆ alkynyl, C₁-C₆ haloalkyl, C₂-C₆ haloalkenyl, C₃-C₈ cycloalkyl, C₁-C₆ alkylthio, C₁-C₆ haloalkylthio, C₁-C₆ alkoxy and C₁-C₆ haloalkoxy;
or salts, stereoisomers, tautomers, polymorphs, metal complexes or N-oxides thereof.

2. The compound of formula (I) according to claim 1 wherein D is Di.

3. The compound of formula (I) according to claim 1 wherein D is D₂.

4. The compound of formula (I) according to claim 1 wherein D is D₃.

5. The compound of formula (I) according to claim 1 wherein Z is a direct bond.

6. The compound of formula (I) according to claim 1 wherein, D is Di;
Z is a direct bond or —C(═O)—;
R³ is selected from the group consisting of hydrogen, halogen, cyano, C₁-C₆ alkyl, C₁-C₆ haloalkyl, and C₁-C₆ alkoxy;
R⁴ is selected from the group consisting of C₁-C₆ alkyl, C₃-C₈ cycloalkyl, C₃-C₈ cycloalkyl-C₁-C₆ alkyl and —NR^{c}R^{d};
wherein, R^{c} is selected from the group consisting of hydrogen or C₁-C₆ alkyl;
R^{d} is selected from the group consisting of hydrogen or C₁-C₆ alkyl;
R⁵ is selected from the group consisting of C₁-C₆ alkyl, C₁-C₆ haloalkyl, C₃-C₈ cycloalkyl, C₃-C₈ cycloalkyl-C₁-C₆ alkyl, phenyl, benzyl, and C₂-C₆ heterocyclyl;
or
R⁴ and R⁵ substituents together with the atom to which they are attached or together with further atoms selected from the group consisting of C, N, O, C(═O), C(═S), and S(O)₀₋₂ may form a 4- to 6-membered ring, which for its part may optionally be substituted by one or more groups consisting of halogen, CN and C₁-C₆ alkyl;
wherein each aliphatic group of R⁴ and R⁵ may be optionally substituted with one or more groups of R^{a} and cyclic groups of R⁴ and R⁵ may optionally be substituted with one or more groups of R^{b},
R^{a} is selected from the group consisting of halogen, cyano, C₁-C₆ alkyl, C₁-C₆ haloalkyl, C₃-C₈ cycloalkyl, C₁-C₆ alkoxy, C₁-C₆ alkylthio, phenyl and C₂-C₆ heterocyclyl;
R^{b} is selected from the group consisting of halogen, cyano, C₁-C₆ alkyl, C₂-C₆ alkenyl, C₂-C₆ alkynyl, C₁-C₆ haloalkyl, C₂-C₆ haloalkenyl, C₃-C₈ cycloalkyl, C₁-C₆ alkylthio, C₁-C₆ haloalkylthio, C₁-C₆ alkoxy and C₁-C₆ haloalkoxy;
R⁸ and R^{8a} are independently selected from the group consisting of hydrogen, halogen, cyano, C₁-C₄-alkyl, and C₁-C₄-haloalkyl;
A₁, A₂, A₃, A₄, and A₅ are same as defined in claim 1;
or salts, stereoisomers, tautomers, polymorphs, metal complexes or N-oxides thereof.

7. The compound of formula (I) according to claim 1 wherein, D is D₂; Z is a direct bond;
R³ is selected from the group consisting of hydrogen, halogen, C₁-C₆ alkyl, C₁-C₆ haloalkyl, and C₁-C₆ alkoxy;
R⁶ is selected from the group consisting of C₁-C₆ alkyl, C₃-C₈ cycloalkyl, C₃-C₈ cycloalkyl-C₁-C₆ alkyl and —NR^{c}R^{d};
wherein, R^{c} is selected from the group consisting of hydrogen or C₁-C₆ alkyl;
R^{d} is selected from the group consisting of hydrogen or C₁-C₆ alkyl;
R⁷ is selected from the group consisting of hydrogen, C₁-C₆ alkyl, C₁-C₆ haloalkyl, C₃-C₈ cycloalkyl, C₃-C₈ cycloalkyl-C₁-C₆ alkyl, —COR⁵, phenyl, benzyl, C₂-C₆ heterocyclyl and cyano;
wherein, R⁵ is selected from the group consisting of C₁-C₆ alkyl and C₃-C₈ cycloalkyl;
wherein each aliphatic group of R⁵, R⁶ and R⁷ may be optionally substituted with one or more groups of R^{a} and cyclic groups of R⁵, R⁶ and R⁷ may optionally be substituted with one or more groups of R^{b},
R^{a} is selected from the group consisting of halogen, cyano, C₁-C₆ alkyl, C₁-C₆ haloalkyl, C₃-C₈ cycloalkyl, C₁-C₆ alkoxy, C₁-C₆ alkylthio, phenyl and C₂-C₆ heterocyclyl;
R^{b} is selected from the group consisting of halogen, cyano, C₁-C₆ alkyl, C₂-C₆ alkenyl, C₂-C₆ alkynyl, C₁-C₆ haloalkyl, C₂-C₆ haloalkenyl, C₃-C₈ cycloalkyl, C₁-C₆ alkylthio, C₁-C₆ haloalkylthio, C₁-C₆ alkoxy and C₁-C₆ haloalkoxy;
R⁸ and R^{8a} are independently selected from the group consisting of hydrogen, halogen, cyano, C₁-C₄-alkyl, and C₁-C₄-haloalkyl;
A₁, A₂, A₃, A₄, and A₅ are same as defined in claim 1;
or salts, stereoisomers, tautomers, polymorphs, metal complexes or N-oxides thereof;

8. The compound of formula (I) according to claim 1 wherein, D is D₃; Z is a direct bond;
R¹ is selected from C₁-C₆ alkyl, C₂-C₆ alkenyl, C₂-C₆ alkynyl, C₁-C₆ haloalkyl, C₃-C₆ cycloalkyl, C₃-C₆ cycloalkyl-C₁-C₆ alkyl, phenyl, benzyl, phenyl ethyl and C₂-C₆ heterocyclyl;
wherein each aliphatic group of R¹ may be optionally substituted with one or more groups of R^{a} and cyclic groups of R¹ may optionally be substituted with one or more groups of R^{b},
R^{a} is selected from the group consisting of halogen, cyano, C₁-C₆ alkyl, C₁-C₆ haloalkyl, C₃-C₈ cycloalkyl, C₁-C₆ alkoxy, C₁-C₆ alkylthio, phenyl and C₂-C₆ heterocyclyl;
R^{b} is selected from the group consisting of halogen, cyano, C₁-C₆ alkyl, C₂-C₆ alkenyl, C₂-C₆ alkynyl, C₁-C₆ haloalkyl, C₂-C₆ haloalkenyl, C₃-C₈ cycloalkyl, C₁-C₆ alkylthio, C₁-C₆ haloalkylthio, C₁-C₆ alkoxy and C₁-C₆ haloalkoxy;
R² is C₁-C₆ alkyl;
R³ is selected from the group consisting of hydrogen, halogen, C₁-C₆ alkyl, C₁-C₆ haloalkyl, and C₁-C₆ alkoxy;
R⁸ and R^{8a} are independently selected from the group consisting of hydrogen, halogen, cyano, C₁-C₄-alkyl, and C₁-C₄-haloalkyl;
A₁, A₂, A₃, A₄, and A₅ are same as defined in claim 1;
or salts, stereoisomers, tautomers, polymorphs, metal complexes or N-oxides thereof.

9. The compound of formula (I) according to claim 1, wherein A₁, A₂, A₃, A₄ and A₅ are independently C; or A₁, A₂, A₃ and A₅ are independently C, and A₄ is N; or A₁, A₂, A₄ and A₅ are independently C, and A₃ is N; or A₁, A₂ and A₅ are independently C, and A₃ and A₄ are independently N; or A₂, A₃, A₄, and A₅ are independently C and A₁ is N; or A₂, A₃ and A₅ are independently C, and A₁ and A₄ are independently N; or A₅ is N and A₁, A₂, A₃, and A₄ are independently C; or A₂ is N and A₁, A₃, A₄ and A₅ are independently C; or A₂ and A₄ are independently N and A₁, A₃ and A₅ are independently C.

10. A method for the preparation of compound of formula (I) or salts, metal complexes, stereoisomers, polymorphs or N-oxides thereof according to claim 1 wherein D is D₁ or D₃ comprising the steps of:
i. reacting a compound of formula (A-1) with a compound of formula (D₁-1) or (D₃-1) to obtain a compound of formula (I) wherein Z is a direct bond; 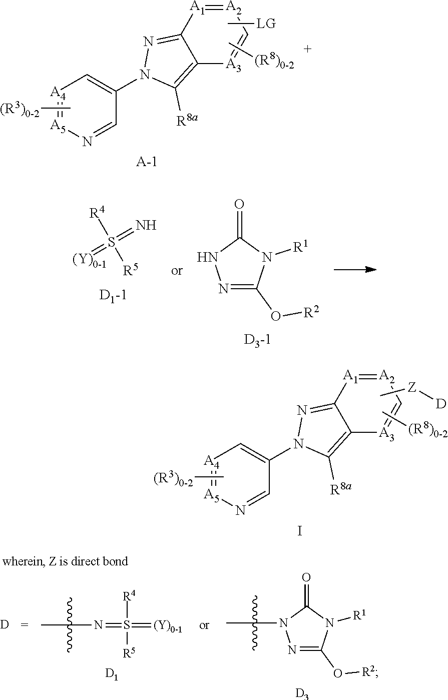
i. reacting a compound of formula (A-1) with a compound of formula (D₁-1) or (D₃-1) to obtain a compound of formula (I) wherein Z is a —C(═O)— bond; 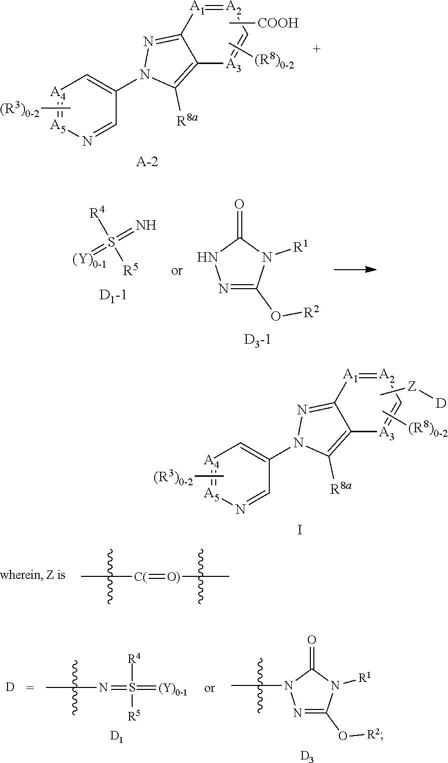
wherein, LG represents a leaving group and the definition of A₁, A₂, A₃, A₄, A₅, R¹, R², R³, R⁴, R⁵, R⁸ and R^{8a} are same as defined in claim 1.

11. A method for the preparation of compound of formula (I) or salts, metal complexes, stereoisomers, polymorphs or N-oxides thereof according to claim 1 wherein D is D₂ comprises the steps of:
i. reacting a compound of formula (A-1) with a compound of formula R⁶SH to obtain a compound of formula (A-3) wherein Z is a direct bond; 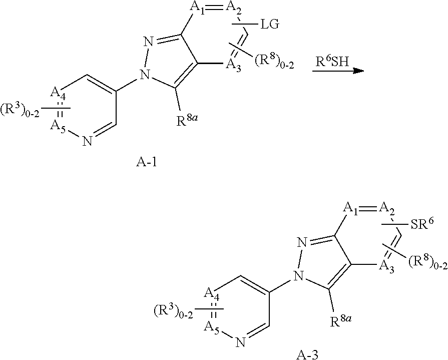
ii. converting the compound of formula (A-3) to a compound of formula (I) through (O) and (N) transfer using a suitable oxidizing reagents and N-sources to obtain a compound of formula (I), 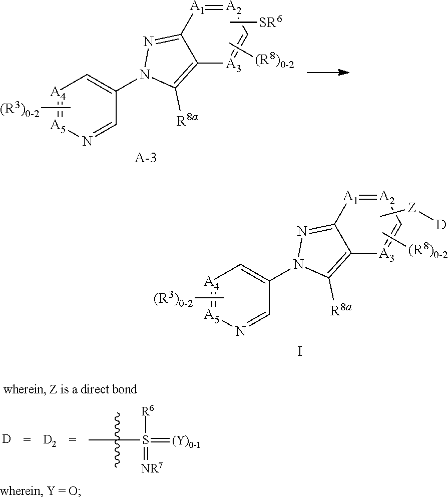
wherein, the definition of A₁, A₂, A₃, A₄, A₅, R³, R⁶, R⁷, R⁸ and R^{8a}, are same as defined in claim 1.

12. A composition comprising the compound of formula (I), its salts, metal complexes, stereoisomers, polymorphs or N-oxides thereof according to claim 1 and at least one additional component selected from the group consisting of surfactants and auxiliaries.

13. The composition according to claim 12, wherein the said composition additionally comprises at least one biological active compatible compound selected from fungicides, insecticides, nematicides, acaricides, biopesticides, herbicides, plant growth regulators, biostimulants, antibiotics, fertilizers or nutrients.

14. The composition according to claim 12, wherein the said compound of formula (I) is present in an amount ranging from 0.1% to 99% by weight with respect to the total weight of the composition.

15. A combination comprising a biologically effective amount of the compound of formula (I) according to claim 1 and at least one additional biological active compatible compound selected from fungicides, insecticides, nematicides, acaricides, biopesticides, herbicides, plant growth regulators, biostimulants, antibiotics, fertilizers and nutrients.

16. A method for combating insects and mite pests comprising contacting the insects and mite pests, their habitat, breeding ground, food supply, plant, seed, soil, area, material or environment in which the insect and mite pests are growing or may grow, or the materials, plants, seeds, soils, surfaces or spaces to be protected from a pest attack or an infestation with a biologically effective amount of a compound of formula (I), salts, stereoisomers, polymorphs, metal complexes, N-oxides, composition or combination thereof according to claim 1, wherein the method comprises applying effective dosages of the compound of formula (I) in amounts ranging from 1 gai to 5000 gai per hectare in agricultural or horticultural crops.

17. A method for protecting crops from an attack or infestation by insects and mite pests comprises contacting the crop with the compound of formula (I), salts, stereoisomers, polymorphs, metal complexes, N-oxides, composition or combination thereof according to claim 1, wherein the method comprises applying effective dosages of the compound of formula (I) in amounts ranging from 1 gai to 5000 gai per hectare in agricultural or horticultural crops.

18. (canceled)

19. A method for the protection of seeds, plants and plant parts from soil insects and of the seedlings roots and shoots from soil and foliar insects comprising contacting the seeds before sowing and/or after pre-germination with the compound of formula (I), salts, stereoisomers, polymorphs, metal complexes, N-oxides, composition or combination thereof according to claim 1.

20. (canceled)

21. (canceled)

22. A seed comprising a compound of formula (I) or salts, metal complexes, N-oxides, stereoisomers, polymorphs, composition or combination thereof according to claim 1, wherein the amount of the compound of formula (I) in the said seed is ranging from 0.0001% to 1% by weight.
